(19) United States
(12) Patent Application Publication
(10) Pub. No.: US 2025/0091994 A1
(43) Pub. Date: Mar. 20, 2025

(54) RAS/RAF BINDING INHIBITOR COMPOUND

(71) Applicant: NATIONAL UNIVERSITY CORPORATION KOBE UNIVERSITY, Hyogo (JP)

(72) Inventors: Fumi SHIMA, Hyogo (JP); Yoko YOSHIKAWA, Hyogo (JP); Shigeyuki MATSUMOTO, Hyogo (JP); Yoshiteru MAKINO, Hyogo (JP); Hirokazu KUBOTA, Saitama (JP); Hitomi YUKI, Saitama (JP); Takashi KUMASAKA, Hyogo (JP); Takashi KAWAMURA, Hyogo (JP); Ko KIKUZATO, Saitama (JP)

(73) Assignee: NATIONAL UNIVERSITY CORPORATION KOBE UNIVERSITY, Hyogo (JP)

(21) Appl. No.: 18/292,565

(22) PCT Filed: Jul. 29, 2022
(86) PCT No.: PCT/JP2022/029379

§ 371 Date: Oct. 11, 2024

(30) Foreign Application Priority Data
Jul. 30, 2021 (JP) 2021-125013

(51) Int. Cl. C07D 209/36; A61K 31/404; A61K 31/4439; A61K 31/4709; A61P 35/00; C07D 401/06; C07D 403/06; C07D 403/12

(52) U.S. Cl. CPC C07D 209/36; A61K 31/404; A61K 31/4439; A61K 31/4709; A61P 35/00; C07D 401/06; C07D 403/06; C07D 403/12

(57) ABSTRACT

The object of the present invention is to provide a Ras/Raf binding inhibitory compound exhibiting a Ras/Raf signaling inhibitory action even against cancer cells bearing proved drug resistance and those with a variety of Ras mutations. The Ras/Raf binding inhibitory compound of the present invention is represented by the following formula (I) (wherein A, B, X, and R¹ to R⁴ are as defined in the present description).

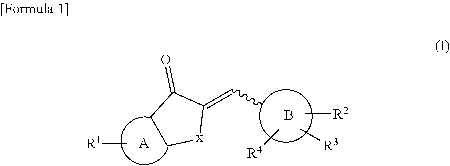

### TECHNICAL FIELD

The present invention relates to: a novel excellent low-molecular-weight compound that specifically inhibits Ras/Raf binding; a pharmaceutical composition comprising the same; and a method for producing the above-described pharmaceutical composition, using such a compound.

It has been reported that the Ras/Raf/MEK/ERK signaling pathway (Ras/MAPK pathway) is activated in many cancers including, as typical examples, leukemia (ALL, APL, and AML), small cell lung cancer, colorectal cancer, pancreatic cancer, and melanoma.

Thus, the present compound and the present pharmaceutical composition provide a novel and excellent therapeutic method for these cancers.

### BACKGROUND ART

Small GTPase Ras, consisting of three isoforms, H-Ras, K-Ras and N-Ras, is one of the most frequently mutated oncogenes found in about 25% of human cancers, and plays a major role in cancer development and progression (Non-Patent Document 1 and Non-Patent Document 2).

The oncogenic potential of Ras is mainly due to point mutations at codons 12, 13, and 61. These point mutations impair GTP hydrolytic activity originally possessed by Ras, and allow Ras to be insensitive to the action of GAPs (GTP hydrolysis-promoting factors), thereby leading to the abundance of Ras-GTP in cells.

The abundant Ras-GTP, through its interaction with three major target proteins, Raf kinases including c-Raf-1 and B-Raf, PI3K, and RalGDS (Ral guanine nucleotide exchange factor) family proteins, induces and activates structural changes in these proteins, thereby causing tumorigenesis in cells and tissues (Non-Patent Document 3, Non-Patent Document 4, and Non-Patent Document 5).

Despite decades of dedicated efforts on the research and development for targeting the clinically prevalent Ras mutants, no successful development has been achieved for effective molecular-targeted cancer therapeutics that can comprehensively inhibit the activity of Ras bearing diverse mutations, especially activated Ras-GTP, that is essential for cancers (Non-Patent Document 2, Non-Patent Document 6, and Non-Patent Document 7).

On the other hand, Shokat et al. have partially overcome this obstacle by identifying electrophilic compounds that covalently bind to cysteine 12 of the mutant K-RasG12C-GDP (inactive form), thereby preventing GTP-bound active form production, that is mediated by the upstream regulator, nucleotide exchange factor Sos (Non Patent Document 8).

K-RasG12C-GDP-specific inhibitors with improved physicochemical properties, such as AMG510 and MRTX849, are currently in clinical trials as a monotherapy or a combination therapy with immune checkpoint inhibitors or other low-molecular-weight inhibitors (Non-Patent Document 6 and Non-Patent Document 7), or several K-RasG12C-GDP-specific inhibitors have reached the market (https://www.fda.gov/news-events/press-announcements/fda-approves-first-targeted-therapy-lung-cancer-mutation-previously-considered-resistant-drug)(https://www.nature.com/articles/d41573-021-00098-4;doi: https://doi.org/10.1038/d41573-021-00098-4)).

Even though K-RasG12C-GDP-specific inhibitors have exhibited quite promising early signs in clinical trials of patients with advanced K-RasG12C mutant lung cancer, namely, clear responses in about half of the patients (https://investors.amgen.com/news-releases/news-release-details/amgen-announces-new-clinical-data-evaluating-novel-0; https://ir.mirati.com/press-releases/press-release-details/2021/Mirati-Therapeutics-Reports-First-Quarter-2021-Financial-Results-and-Recent-Corporate-Updates/default.aspx), “drug resistance exists in these inhibitors,” as Aaron and Shaw have noted.

Furthermore, it should be noted once again that even the latest research and development have not been able to comprehensively overcome clinically frequent Ras mutations such as G12D, G12V, and G13D.

Summing up these findings, it is found that there is an urgent need for the development of broad-spectrum Ras inhibitors or effective strategies to more comprehensively inhibit Ras signaling (Non-Patent Documents 9 to 11).

### PRIOR ART DOCUMENTS

### Non-Patent Documents

Non-Patent Document 1:
Simanshu D K, Nissley D V. McCormick F. RAS proteins and their regulators in human disease. Cell 2017, 170: 17-33
Non-Patent Document 2:
Stephen A G, Esposito D, Bagni R K, McCormick F. Dragging ras back in the ring. Cancer cell. 2014, Mar. 17; 25(3): 272-81
Non-Patent Document 3:
Vetter I R, Wittinghofer A. The guanine nucleotide-binding switch in three dimensions. Science 2001, 294: 1299-1304
Non-Patent Document 4:
Weinstein B. Addiction to oncogenes—the Achilles heel of cancer. Science 2002, 297: 63-64
Non-Patent Document 5:
Knrnoub A E, Weinberg R A. Ras oncogenes: split personalities. Nat Rev Mol Cell Biol 2008, 9: 517-531
Non-Patent Document 6:
Mullard A. Cracking KRas. Nat. Rev. Drug Discov. 2019, December: 887-891
Non-Patent Document 7:
Sheridan C. Grail of RAS cancer drugs within reach. Nature Biotech. 2020, 38: 6-8
Non-Patent Document 8:
Ostrem J M, Peters U, Sos M L, Wells J A and Shokat K M. K-Ras (G12C) inhibitors allosterically control GTP affinity and effector interactions. Nature 2013, 503: 548-551
Non-Patent Document 9:
Merchant M, Moffat J, Schaefer G, Chan J, Wang X, Orr C, et al. Combined MEK and ERK inhibition overcomes therapy-mediated pathway reactivation in RAS mutant tumors. PLoS One 2017; 12: e0185862
Non-Patent Document 10:
Chandarlapaty S, Sawai A, Scaltriti M, Rodrik-Outmezguine V, Grbovic-Huezo O, Serra V, et al. AKT inhibition relieves feedback suppression of receptor tyrosine kinase expression and activity. Cancer Cell 2011; 19: 58-71
Non-Patent Document 11:
Misale S, Fatherree J P, Cortez E, Li C, Bilton S J, Timonina D, et al. KRAS G12C NSCLC models are sensitive to direct targeting of KRAS in combination with PI3K inhibition. Clin Cancer Res 2019; 25: 769-807

### SUMMARY OF INVENTION

### Object to be Solved by the Invention

As described above, there have been Raf inhibitors already applied in clinical use for cancers involving Ras/MAPK pathway activation, and drugs in clinical development or already approved for cancers with RasG12C mutations. However, these drugs have issues to be addressed, such as induction of drug resistance and limited efficacy shown in a few cancer types.

It is an object of the present invention to provide a Ras/Raf binding inhibitory compound, which exhibits a Ras/Raf signaling inhibitory action even against cancer with drug resistance and those with a wide variety of Ras mutants.

### Means for Solving the Object

The present inventors have conducted intensive studies regarding the synthesis of a derivative specifically acting on Ras/Raf signaling inhibition, and a pharmaceutical activity thereof, over a long period of years. As a result, they have found that a compound represented by the following formula (I) has an excellent Ras/Raf signaling inhibitory action, thereby completing the present invention.

(1) As an aspect of the present invention, a compound represented by the following formula (I) or a pharmaceutically acceptable salt thereof, or an isomer thereof, is provided:

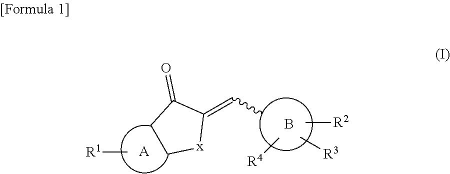

wherein
A represents a benzene ring or a pyridine ring,
B represents a C₆₋₁₀ aryl group, or a heteroaryl group containing 1 to 4 atoms selected from N, S and O,
X represents —NR—,
R¹ represents H, a C₁₋₆ alkyl group optionally substituted with a NHCOCH₃ group, or halogen,
R², R³ and R⁴, which are the same or different, each represent (provided that at least any one of them is not H)
H;
C₁₋₆ alkyl-SO₂— (e.g. mesyl, etc.);
cyano;
halogen;
nitro;
azide;
a —CO—R¹¹ group (wherein the R¹¹ represents
OH,
an R¹² group (wherein the R¹² group represents a heterocyclyl group containing 1 or 2 atoms (groups) selected from N, S, SO, SO₂ and O, and optionally having a substituent),
NH₂, NHR¹², or N(R¹²)R¹², or
C₆₋₁₀ arylamino);
an —O—CH₂—CO—R¹¹ group;
a C₁₋₆ alkyl group optionally having a substituent
(wherein the substituent represents
   —CONH₂,
   an R¹² group,
   OH,
   —NR¹³R¹⁴ (wherein the R¹³ represents C₁₋₆ alkyl-SO₂— (e.g. mesyl, etc.), C₁₋₆ alkyl-CO— (preferably, acetyl), or an R¹² group, and the R¹⁴ represents H, a C₁₋₆ alkyl group, or an R¹² group),
   R¹²—CO—, or
   R¹²—C₁₋₆ alkyl-CONH—);
   an —OR¹⁵ group
(wherein the R¹⁵ represents
   H,
   a C₁₋₆ alkyl group optionally having a substituent (wherein the substituent represents OH, C₁₋₆ alkoxy, C₆₋₁₀ aryl, C₆₋₁₀ aryloxy, R¹², cyano, C₁₋₆ alkyl-SO₂— (e.g. mesyl, etc.), or methylsulfinyl),
   C₁₋₆ alkyl-SO₂— (e.g. mesyl, etc.), or
   a heteroaryl group containing 1 to 4 atoms selected from N, S and O);
   an —NR¹⁶R¹⁷ group
(wherein the R¹⁶ and R¹⁷, which are the same or different, each represent
   H,
   C₁₋₆ alkyl-CO—,
   an R¹² group, or
   R¹²—C₁₋₆ alkyl-CO—,
or
R¹⁶ and R¹⁷ may form an R¹² group, together with N);
   a C₆₋₁₀ aryl group optionally having a substituent
(wherein the substituent represents C₆₋₁₀ aryloxy, a heteroaryl group containing 1 to 4 atoms selected from N, S and O, and optionally having a substituent, or phenyl optionally annelated with R¹²);
   a heteroaryl group containing 1 to 4 atoms selected from N, S and O, and optionally having a substituent; or
   —(CH═CH)—R¹⁸ (wherein the R¹⁸ represents —CO—R¹⁹ or —SO₂—R¹⁹, and the R¹⁹ represents NH₂, or an R¹² group); wherein
      R⁵ represents C₁₋₆ alkyl, —COR⁶, —COOR⁶, —CONR⁶R⁷, —SOnR⁶, or SOnNR⁶R⁷, wherein n represents 0, 1 or 2, and
      R⁶ and R⁷, which are the same or different, each represent H, a C₁₋₆ alkyl group, or a C₆₋₁₀ aryl group, and
      the wavy line represents a geometrical isomer.

Besides, there is a case where “●” and “−” are placed at the beginning of each option in the same group, when multiple options are listed.

(2) As a preferred aspect of the present invention, it is the compound represented by the formula (I) according to the above (1) or a pharmaceutically acceptable salt thereof, or an isomer thereof, wherein A represents a benzene ring.

(3) As a preferred aspect of the present invention, it is the compound represented by the formula (I) according to the above (1) or (2) or a pharmaceutically acceptable salt thereof, or an isomer thereof, wherein B represents a heteroaryl group containing 1 to 4 atoms selected from N, S and O.

(4) As a more preferred aspect of the present invention, it is the compound represented by the formula (I) according to the above (1) or (2) or a pharmaceutically acceptable salt thereof, or an isomer thereof, wherein B represents pyridyl, quinolyl, indolyl, thiazolyl, pyrrolopyridinyl, benzothiazolyl, or furopyridinyl.

(5) As a more preferred aspect of the present invention, it is the compound represented by the formula (I) according to the above (1) or (2) or a pharmaceutically acceptable salt thereof, or an isomer thereof, wherein B represents the following:

### [Formula 2]

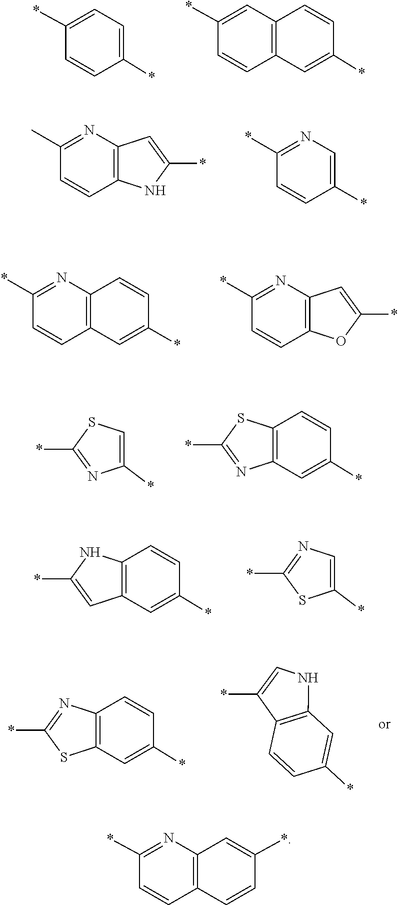

In the above formula, one of the bonding hands corresponds to the bonding of the wavy line in the formula (I), and the other bonding hand represents a bonding hand with any one substituent other than the hydrogen in R², R³ and R⁴.

Besides, in such a case, if there is another substituent, the substituent is substituted with the residual position described above.

(6) As a more preferred aspect of the present invention, it is the compound represented by the formula (I) according to the above (1) or (2) or a pharmaceutically acceptable salt thereof, or an isomer thereof, wherein B is quinolyl, thiazolyl, or benzothiazolyl.

(7) As a preferred aspect of the present invention, it is the compound represented by the formula (I) according to any one of the above (1) to (6) or a pharmaceutically acceptable salt thereof, or an isomer thereof, wherein the R¹ represents H.

(8) As a preferred aspect of the present invention, it is the compound represented by the formula (I) according to any one of the above (1) to (7) or a pharmaceutically acceptable salt thereof, or an isomer thereof, wherein the R¹¹ of the —CO—R¹¹ group as represented in R², R³ and R⁴ represents OH or an R¹² group.

(9) As a preferred aspect of the present invention, it is the compound represented by the formula (I) according to any one of the above (1) to (7) or a pharmaceutically acceptable salt thereof, or an isomer thereof, wherein the R¹¹ of the —O—CH₂—CO—R¹¹ group as represented in R², R³ and R⁴ represents an R¹² group.

(10) As a preferred aspect of the present invention, it is the compound represented by the formula (I) according to any one of the above (1) to (7) or a pharmaceutically acceptable salt thereof, or an isomer thereof, wherein the substituent of the C₁₋₆ alkyl group optionally having a substituent, as represented in R², R³ and R⁴, represents an R¹² group, OH, —NR¹³R¹⁴ (wherein the R¹³ represents C₁₋₆ alkyl-SO₂— (e.g. mesyl, etc.) or an R¹² group, and the R¹⁴ represents H or a C₁₋₆ alkyl group), R¹²—CO—, or R¹²—C₁₋₆ alkyl-CONH—.

(11) As a more preferred aspect of the present invention, it is the compound represented by the formula (I) according to the above (10) or a pharmaceutically acceptable salt thereof, or an isomer thereof, wherein the substituent of the C₁₋₆ alkyl group optionally having a substituent, as represented in R², R³ and R⁴, represents —NR¹³R¹⁴ (wherein the R¹³ represents C₁₋₆ alkyl-SO₂— (e.g. mesyl, etc.), and the R¹⁴ represents H).

(12) As a preferred aspect of the present invention, it is the compound represented by the formula (I) according to any one of the above (1) to (7) or a pharmaceutically acceptable salt thereof, or an isomer thereof, wherein the R¹⁵ of the —OR¹⁵ group as represented in R², R³ and R⁴ represents a C₁₋₆ alkyl group (preferably, methyl) or C₁₋₆ alkyl-SO₂— (e.g. mesyl, etc.).

(13) As a preferred aspect of the present invention, it is the compound represented by the formula (I) according to any one of the above (1) to (7) or a pharmaceutically acceptable salt thereof, or an isomer thereof, wherein the R¹⁶ and R¹⁷ of the —NR¹⁶R¹⁷ group as represented in R², R³ and R⁴, which are the same or different, each represent H or an R¹² group, or the R¹⁶ and R¹⁷ together represent an R¹² group (preferably, acetylpiperazinyl and cyanopiperidino).

(14) As a preferred aspect of the present invention, it is the compound represented by the formula (I) according to any one of the above (1) to (7) or a pharmaceutically acceptable salt thereof, or an isomer thereof, wherein the C₆₋₁₀ aryl group of the C₆₋₁₀ aryl group optionally having a substituent, as represented in R², R³ and R⁴, represents phenyl or naphthyl, and the substituent represents a heteroaryl group containing 1 to 4 atoms selected from N, S and O, and optionally having a substituent (preferably, pyridyl, phenylpyridyl, quinolyl, indazolyl, pyrazolyl, or methylpyrazolyl; more preferably, indazolyl, pyrazolyl, or methylpyrazolyl), or phenyl optionally annelated with an R¹² group (preferably, phenyl).

(15) As a preferred aspect of the present invention, it is the compound represented by the formula (I) according to any one of the above (1) to (7) or a pharmaceutically acceptable salt thereof, or an isomer thereof, wherein the heteroaryl group containing 1 to 4 atoms selected from N, S and O, and optionally having a substituent, as represented in R², R³ and R⁴, represents pyridyl, phenylpyridyl, quinolyl, indazolyl, pyrazolyl, or methylpyrazolyl.

(16) As a more preferred aspect of the present invention, it is the compound represented by the formula (I) according to the above (15) or a pharmaceutically acceptable salt thereof, or an isomer thereof, wherein the heteroaryl group containing 1 to 4 atoms selected from N, S and O, and optionally having a substituent, as represented in R², R³ and R⁴, represents indazolyl, pyrazolyl, or methylpyrazolyl.

(17) As a preferred aspect of the present invention, it is the compound represented by the formula (I) according to any one of the above (1) to (7) or a pharmaceutically acceptable salt thereof, or an isomer thereof, wherein at least one of R², R³ and R⁴ represents —(CH═CH)—R¹⁸ (wherein the R¹⁸ represents —CO—R¹⁹, and the R¹⁹ represents an R¹² group).

(18) As a preferred aspect of the present invention, it is the compound represented by the formula (I) according to any one of the above (1) to (17) or a pharmaceutically acceptable salt thereof, or an isomer thereof, wherein the heterocyclyl group containing 1 or 2 atoms (groups) selected from N, S, SO, SO₂ and O, in the heterocyclyl group containing 1 or 2 atoms (groups) selected from N, S, SO, SO₂ and O, and optionally having a substituent, as represented in R¹² represents morpholino, piperazinyl, thiomorpholino, dioxidothiomorpholino, tetrahydropyranyl, tetrahydrothiopyranyl, pyrrolidinyl, dioxidotetrahydrothiopyranyl, or piperidinyl.

(19) As a more preferred aspect of the present invention, it is the compound represented by the formula (I) according to the above (18) or a pharmaceutically acceptable salt thereof, or an isomer thereof, wherein the heterocyclyl group containing 1 or 2 atoms (groups) selected from N, S, SO, SO₂ and O, in the heterocyclyl group containing 1 or 2 atoms (groups) selected from N, S, SO, SO₂ and O, and optionally having a substituent, as represented in R¹², represents morpholino, piperazinyl, piperidinyl, or tetrahydropyranyl.

(20) As a preferred aspect of the present invention, it is the compound represented by the formula (I) according to any one of the above (1) to (19) or a pharmaceutically acceptable salt thereof, or an isomer thereof, wherein the substituent in the heterocyclyl group containing 1 or 2 atoms (groups) selected from N, S, SO, SO₂ and O, and optionally having a substituent, as represented in R¹², represents —CO—, —COOH, cyano, 1 or 2 C₁₋₆ alkyl groups (preferably, methyl groups on both sides of the heteroatom selected from N, S, SO, SO₂ and O of the heterocyclyl group, more preferably, 2,6-dimethyl for tetrahydropyranyl and morpholino), C₁₋₆ alkyl-CO— (preferably, acetyl), C₁₋₆ alkyl-SO₂— (e.g. mesyl, etc.), a C₆₋₁₀ aryl group (preferably, phenyl), 3-methoxy-2-hydroxypropyl, an R¹² group (preferably, oxetanyl or morpholino), R¹²—CH₂-(preferably, methoxyoxetanylmethyl), R¹²—CH₂CO— (preferably, morpholinomethylcarbonyl), R¹²—CH₂OCO— (preferably, methoxyoxetanylmethoxycarbonyl), or methoxyethyl.

(21) As a preferred aspect of the present invention, it is the compound represented by the formula (I) according to any one of the above (1) to (20) or a pharmaceutically acceptable salt thereof, or an isomer thereof, wherein the R⁵ represents —COR⁶, and the R⁶ represents a C₁₋₆ alkyl group.

(22) As a preferred aspect, the compound (I) of the present invention or a pharmaceutically acceptable salt thereof, or an isomer thereof, is a compound selected from the compounds shown below.

TABLE 1

| | |
|---|---|
| 1 | (Z)-2-(4-((1-acetyl-3-oxoindolin-2-ylidene)methyl)-2- |
| | methoxyphenoxy)acetamide |
| 2 | (Z)-1-acetyl-2-(3-methoxy-4-(2-morpholino-2- |
| | oxoethoxy)benzylidene)indolin-3-one |
| 3 | 1-acetyl-2-(4-hydroxy-3-methoxybenzylidene)indolin-3-one |
| 4 | 2-(4-((1-acetyl-3-oxoindolin-2-ylidene)methyl)-2- |
| | fluorophenoxy)acetamide |
| 5 | 2-(4-((1-acetyl-3-oxoindolin-2-ylidene)methyl)phenoxy)acetamide |
| 6 | 2-(3-((1-acetyl-3-oxoindolin-2-ylidene)methyl)phenoxy)acetamide |
| 7 | (Z)-1-acetyl-2-(3-methoxy-4-(2- |
| | morpholinoethoxy)benzylidene)indolin-3-one |
| 8 | 2-(2-((1-acetyl-3-oxoindolin-2-ylidene)methyl)phenoxy)acetamide |
| 9 | 2-(4-((1-acetyl-3-oxoindolin-2-ylidene)methyl)-2- |
| | methoxyphenoxy)acetonitrile |
| 10 | 1-acetyl-2-(4-(benzyloxy)-3-methoxybenzylidene)indolin-3-one |
| 11 | 1-acetyl-2-(3-methoxy-4-(2-methoxyethoxy)benzylidene)indolin-3- |
| | one |
| 12 | 1-acetyl-2-(3-methoxy-4-(2-phenoxyethoxy)benzylidene)indolin-3- |
| | one |
| 13 | 2-(4-((1-acetyl-3-oxoindolin-2-ylidene)methyl)-2- |
| | ethoxyphenoxy)acetamide |
| 14 | 1-acetyl-2-(3-methoxy-4-(2-oxo-2-(pyrrolidin-1- |
| | yl)ethoxy)benzylidene)indolin-3-one |
| 15 | 2-(4-((1-acetyl-3-oxoindolin-2-ylidene)methyl)-2-methoxyphenoxy)- |
| | N-phenylacetamide |
| 16 | 1-acetyl-2-(3-fluoro-4-(2-morpholino-2- |
| | oxoethoxy)benzylidene)indolin-3-one |
| 17 | 2-(4-((1-acetyl-3-oxoindolin-2-ylidene)methyl)-2- |
| | chlorophenoxy)acetamide |
| 18 | 1-acetyl-2-(4-(2-morpholino-2-oxoethoxy)-3- |
| | nitrobenzylidene)indolin-3-one |
| 19 | 1-acetyl-2-(3,5-difluoro-4-(2-morpholino-2- |
| | oxoethoxy)benzylidene)indolin-3-one |
| 20 | (2E)-3-(4-((1-acetyl-3-oxoindolin-2-ylidene)methyl)-2- |
| | methoxyphenyl)acrylamide |
| 21 | 4-((1-acetyl-3-oxoindolin-2-ylidene)methyl)-2-methoxyphenyl |
| | methanesulfonate |
| 22 | (Z)-1-acetyl-2-((5-(2-morpholino-2-oxoethoxy)pyridin-2- |
| | yl)methylene)indolin-3-one |
| 23 | 2-(4-((1-acetyl-3-oxoindolin-2-ylidene)methyl)-3- |
| | methoxyphenoxy)acetamide |
| 24 | 1-acetyl-2-(3-methoxy-4- |
| | ((methylsulfonyl)methoxy)benzylidene)indolin-3-one |
| 25 | 1-acetyl-2-(3-methoxy-4- |
| | ((methylsulfinyl)methoxy)benzylidene)indolin-3-one |
| 26 | 2-(4-((1-acetyl-3-oxoindolin-2-ylidene)methyl)-3,5- |
| | dimethoxyphenoxy)acetamide |
| 27 | N-(4-((1-acetyl-3-oxoindolin-2-ylidene)methyl)-2-methoxyphenyl)-2- |
| | morpholinoacetamide |
| 28 | (Z)-1-acetyl-2-((6-(morpholine-4-carbonyl)quinolin-2- |
| | yl)methylene)indolin-3-one |
| 29 | 1-acetyl-2-((6-(2-morpholino-2-oxoethoxy)-[1,1′-biphenyl]-3- |
| | yl)methylene)indolin-3-one |
| 30 | 2-(4-((1-acetyl-3-oxoindolin-2-ylidene)methyl)-2- |
| | isopropoxyphenoxy)acetamide |
| 31 | 3-(4-((1-acetyl-3-oxoindolin-2-ylidene)methyl)-2- |
| | methoxyphenyl)propanamide |
| 32 | (Z)-1-acetyl-2-((6-fluoro-5-(2-morpholino-2-oxoethoxy)pyridin-2- |
| | yl)methylene)indolin-3-one |
| 33 | (Z)-1-acetyl-2-((6-(hydroxymethyl)quinolin-2-yl)methylene)indolin- |
| | 3-one |
| 34 | (Z)-2-((1-acetyl-3-oxoindolin-2-ylidene)methyl)quinoline-6- |
| | carbonitrile |
| 35 | (Z)-1-acetyl-2-((6-(morpholinomethyl)quinolin-2- |
| | yl)methylene)indolin-3-one |
| 36 | (E)-1-acetyl-2-((5-(morpholine-4-carbonyl)-1H-indol-2- |
| | yl)methylene)indolin-3-one |
| 37 | 1-acetyl-2-((6-(morpholine-4-carbonyl)-1H-indol-3- |
| | yl)methylene)indolin-3-one |
| 38 | (Z)-1-acetyl-2-((5-(morpholine-4-carbonyl)thiazol-2- |
| | yl)methylene)indolin-3-one |
| 39 | (Z)-1-acetyl-2-((1-(methylsulfonyl)-1H-pyrrolo[3,2-b]pyridin-5- |
| | yl)methylene)indolin-3-one |
| 40 | (Z)-1-acetyl-2-((2-(morpholine-4-carbonyl)-1H-pyrrolo[3,2- |
| | b]pyridin-5-yl)methylene)indolin-3-one |
| 41 | (Z)-1-acetyl-2-((6-(4-(oxetan-3-yl)piperazine-1-carbonyl)quinolin-2- |
| | yl)methylene)indolin-3-one |
| 42 | (Z)-1-acetyl-2-((6-(4-(2-hydroxy-3-methoxypropyl)piperazine-1- |
| | carbonyl)quinolin-2-yl)methylene)indolin-3-one |
| 43 | (E)-1-acetyl-2-((6-((E)-2-(morpholinosulfonyl)vinyl)benzo[d]thiazol- |
| | 2-yl)methylene)indolin-3-one |
| 44 | (Z)-1-acetyl-2-((6-(2,6-dimethylmorpholine-4-carbonyl)quinolin-2- |
| | yl)methylene)indolin-3-one |
| 45 | (Z)-1-acetyl-2-((6-(3-morpholino-3-oxopropyl)quinolin-2- |
| | yl)methylene)indolin-3-one |
| 46 | (Z)-1-acetyl-2-((6′-(morpholine-4-carbonyl)-[3,4′-biquinolin-2′- |
| | yl)methylene)indolin-3-one |
| 47 | (Z)-1-acetyl-2-((5-(morpholine-4-carbonyl)benzo[d]thiazol-2- |
| | yl)methylene)indolin-3-one |
| 48 | (E)-1-acetyl-2-((5-(morpholine-4-carbonyl)benzo[d]thiazol-2- |
| | yl)methylene)indolin-3-one |
| 49 | 2-(4-((1-benzoyl-3-oxoindolin-2-ylidene)methyl)-2- |
| | methoxyphenoxy)acetamide |
| 50 | 1-acetyl-5-fluoro-2-(3-methoxy-4-(2-morpholino-2- |
| | oxoethoxy)benzylidene)indolin-3-one |
| 51 | 1-acetyl-2-(3-methoxy-4-(2-morpholino-2-oxoethoxy)benzylidene)- |
| | 1,2-dihydro-3H-pyrrolo[2,3-b]pyridin-3-one |
| 52 | (1E)-2-(4-((1-acetyl-3-oxoindolin-2-ylidene)methyl)-2- |
| | methoxyphenyl)ethene-1-sulfonamide |
| 53 | (E)-N-((2-((1-acetyl-3-oxoindolin-2-ylidene)methyl)benzo[d]thiazol- |
| | 5-yl)methyl)methanesulfonamide |
| 54 | (Z)-6-((1-acetyl-3-oxoindolin-2-ylidene)methyl)-2-fluoropyridin-3-yl |
| | methanesulfonate |
| 55 | (E)-1-acetyl-2-((4-(morpholine-4-carbonyl)thiazol-2- |
| | yl)methylene)indolin-3-one |
| 56 | (E)-N-((2-((1-acetyl-3-oxoindolin-2-ylidene)methyl)benzo[d]thiazol- |
| | 6-yl)methyl)methanesulfonamide |
| 57 | 2-(4-((1-acetyl-3-oxoindolin-2-ylidene)methyl)-2- |
| | methoxyphenoxy)aceticacid |
| 58 | (E)-1-acetyl-2-((6-(morpholine-4-carbonyl)benzo[d]thiazol-2- |
| | yl)methylene)indolin-3-one |
| 59 | (Z)-1-acetyl-2-((6-(morpholine-4-carbonyl)benzo[d]thiazol-2- |
| | yl)methylene)indolin-3-one |
| 60 | (Z)-1-acetyl-2-((6-(1,1-dioxidothiomorpholine-4-carbonyl)quinolin-2- |
| | yl)methylene)indolin-3-one |
| 61 | (Z)-6-((1-acetyl-3-oxoindolin-2-ylidene)methyl)pyridin-3-yl |
| | methanesulfonate |
| 62 | (Z)-1-acetyl-2-((7-(morpholine-4-carbonyl)quinolin-2- |
| | yl)methylene)indolin-3-one |
| 63 | (Z)-N-((2-((1-acetyl-3-oxoindolin-2-ylidene)methyl)quinolin-6- |
| | yl)methyl)acetamide |
| 64 | (Z)-N-((2-((1-acetyl-3-oxoindolin-2-ylidene)methyl)quinolin-6- |
| | yl)methyl)methanesulfonamide |
| 65 | (Z)-1-acetyl-2-((6-(2-morpholino-2-oxoethoxy) quinolin-2- |
| | yl)methylene)indolin-3-one |
| 66 | 1-acetyl-2-((6-(morpholine-4-carbonyl)naphthalen-2- |
| | yl)methylene)indolin-3-one |
| 67 | 1-acetyl-2-(4-(morpholine-4-carbonyl)benzylidene)indolin-3-one |
| 68 | (E)-1-acetyl-2-((5-(morpholine-4-carbonyl)thiazol-2- |
| | yl)methylene)indolin-3-one |
| 69 | 1-acetyl-2-((1-methyl-6-(morpholine-4-carbonyl)-1H-indol-3- |
| | yl)methylene)indolin-3-one |
| 70 | (Z)-1-acetyl-2-((5-(morpholine-4-carbonyl)pyridin-2- |
| | yl)methylene)indolin-3-one |
| 71 | (Z)-1-acetyl-2-((6-((methyl(tetrahydro-2H-pyran-4- |
| | yl)amino)methyl)quinolin-2-yl)methylene)indolin-3-one |
| 72 | (Z)-1-acetyl-2-((2-(morpholine-4-carbonyl)furo[3,2-b]pyridin-5- |
| | yl)methylene)indolin-3-one |
| 73 | (Z)-1-acetyl-2-((6-(4-(oxetan-3-yl)piperazin-1-yl)quinolin-2- |
| | yl)methylene)indolin-3-one |
| 74 | (Z)-1-acetyl-2-((6-((4-(oxetan-3-yl)piperazin-1-yl)methyl)quinolin-2- |
| | yl)methylene)indolin-3-one |
| 75 | (Z)-N-((2-((1-acetyl-3-oxoindolin-2-ylidene)methyl)quinolin-6- |
| | yl)methyl)-N-(tetrahydro-2H-pyran-4-yl)acetamide |
| 76 | (Z)-N-((2-((1-acetyl-3-oxoindolin-2-ylidene)methyl)quinolin-6- |
| | yl)methyl)-N-(tetrahydro-2H-pyran-4-yl)methanesulfonamide |
| 77 | (Z)-1-acetyl-2-((6-((E)-3-morpholino-3-oxoprop-1-en-1-yl)quinolin- |
| | 2-yl)methylene)indolin-3-one |
| 78 | (Z)-1-acetyl-2-((6-((E)-3-(4-(oxetan-3-yl)piperazin-1-yl)-3-oxoprop- |
| | 1-en-1-yl)quinolin-2-yl)methylene)indolin-3-one |
| 79 | (E)-3-(2-(((Z)-1-acetyl-3-oxoindolin-2-ylidene)methyl)quinolin-6-yl)- |
| | N-(1-(oxetan-3-yl)piperidin-4-yl)acrylamide |
| 80 | (Z)-N-((2-((1-acetyl-3-oxoindolin-2-ylidene)methyl)quinolin-6- |
| | yl)methyl)-2-morpholinoacetamide |
| 81 | (Z)-1-acetyl-2-((4-azido-6-(morpholine-4-carbonyl)quinolin-2- |
| | yl)methylene)indolin-3-one |
| 82 | (Z)-1-acetyl-2-((4-chloro-6-(morpholine-4-carbonyl)quinolin-2- |
| | yl)methylene)indolin-3-one |
| 83 | (Z)-1-acetyl-2-((6-(morpholine-4-carbonyl)-4-phenylquinolin-2- |
| | yl)methylene)indolin-3-one |
| 84 | (Z)-1-acetyl-2-((4-butoxy-6-(morpholine-4-carbonyl)quinolin-2- |
| | yl)methylene)indolin-3-one |
| 85 | (Z)-1-acetyl-2-((6-(morpholine-4-carbonyl)-4-(pyridin-3-yl)quinolin- |
| | 2-yl)methylene)indolin-3-one |
| 86 | (Z)-2-((1-acetyl-3-oxoindolin-2-ylidene)methyl)-6-(morpholine-4- |
| | carbonyl)quinoline-4-carbonitrile |
| 87 | 1-(2-((1-acetyl-3-oxoindolin-2-ylidene)methyl)-6-(morpholine-4- |
| | carbonyl)quinolin-4-yl)piperidine-4-carbonitrile |
| 88 | (Z)-2-((4-([1,1′-biphenyl]-3-yl)-6-(morpholine-4-carbonyl)quinolin-2- |
| | yl)methylene)-1-acetylindolin-3-one |
| 89 | (Z)-2-((4-([1,1′-biphenyl]-2-yl)-6-(morpholine-4-carbonyl)quinolin-2- |
| | yl)methylene)-1-acetylindolin-3-one |
| 90 | (Z)-1-acetyl-2-((4-(4-acetylpiperazin-1-yl)-6-(morpholine-4- |
| | carbonyl)quinolin-2-yl)methylene)indolin-3-one |
| 91 | (Z)-1-acetyl-2-((6-(morpholine-4-carbonyl)-[4,8′-biquinolin]-2- |
| | yl)methylene)indolin-3-one |
| 92 | (Z)-1-acetyl-2-((6-(morpholine-4-carbonyl)-4-(4- |
| | phenoxyphenyl)quinolin-2-yl)methylene)indolin-3-one |
| 93 | 1-acetyl-2-((1-methyl-5-(morpholine-4-carbonyl)-1H-indol-2- |
| | yl)methylene)indolin-3-one |
| 94 | 1-acetyl-2-((6-(morpholine-4-carbonyl)-4-(2-phenylpyridin-3- |
| | yl)quinolin-2-yl)methylene)indolin-3-one |
| 95 | (Z)-2-((4-([1,1′-biphenyl]-2-yl)-6-(4-(oxetan-3-yl)piperazine-1- |
| | carbonyl)quinolin-2-yl)methylene)-1-acetylindolin-3-one |
| 96 | (Z)-2-((4-([1,1′-biphenyl]-2-yl)-6-(4-(2-hydroxy-3- |
| | methoxypropyl)piperazine-1-carbonyl)quinolin-2-yl)methylene)-1- |
| | acetylindolin-3-one |
| 97 | (Z)-4-([1,1′-biphenyl]-2-yl)-2-((1-acetyl-3-oxoindolin-2- |
| | ylidene)methyl)-N-(1-(oxetan-3-yl)piperidin-4-yl)quinoline-6- |
| | carboxamide |
| 98 | (Z)-1-acetyl-2-((5-(2,6-dimethylmorpholine-4- |
| | carbonyl)benzo[d]thiazol-2-yl)methylene)indolin-3-one |
| 99 | (E)-1-acetyl-2-((5-(2,6-dimethylmorpholine-4- |
| | carbonyl)benzo[d]thiazol-2-yl)methylene)indolin-3-one |
| 100 | (Z)-1-acetyl-2-((6-(2,6-dimethylmorpholine-4- |
| | carbonyl)benzo[d]thiazol-2-yl)methylene)indolin-3-one |
| 101 | (E)-1-acetyl-2-((6-(2,6-dimethylmorpholine-4- |
| | carbonyl)benzo[d]thiazol-2-yl)methylene)indolin-3-one |
| 102 | 1-acetyl-2-((5-(2-morpholino-2-oxoethoxy)pyridin-2-yl)methylene)- |
| | 1,2-dihydro-3H-pyrrolo[2,3-b]pyridin-3-one |
| 103 | 1-acetyl-6-fluoro-2-(3-methoxy-4-(2-morpholino-2- |
| | oxoethoxy)benzylidene)indolin-3-one |
| 104 | 1-acetyl-4-fluoro-2-(3-methoxy-4-(2-morpholino-2- |
| | oxoethoxy)benzylidene)indolin-3-one |
| 105 | 1-acetyl-7-fluoro-2-(3-methoxy-4-(2-morpholino-2- |
| | oxoethoxy)benzylidene)indolin-3-one |
| 106 | (Z)-N-((1-acetyl-2-((6-(morpholine-4-carbonyl)quinolin-2- |
| | yl)methylene)-3-oxoindolin-4-yl)methyl)acetamide |
| 107 | (Z)-1-acetyl-2-((6-((1,1-dioxidothiomorpholino)methyl)quinolin-2- |
| | yl)methylene)indolin-3-one |
| 108 | (E)-1-acetyl-2-((6-((E)-3-morpholino-3-oxoprop-1-en-1- |
| | yl)benzo[d]thiazol-2-yl)methylene)indolin-3-one |
| 109 | (Z)-1-acetyl-2-((5-((E)-3-morpholino-3-oxoprop-1-en-1- |
| | yl)benzo[d]thiazol-2-yl)methylene)indolin-3-one |
| 110 | (E)-1-acetyl-2-((5-((E)-3-morpholino-3-oxoprop-1-en-1- |
| | yl)benzo[d]thiazol-2-yl)methylene)indolin-3-one |
| 111 | (Z)-2-((1-acetyl-3-oxoindolin-2-ylidene)methyl)-6-(morpholine-4- |
| | carbonyl)quinoline-4-carboxamide |
| 112 | (Z)-2-((4-((3H-[1,2,3]triazolo[4,5-b]pyridin-3-yl)oxy)-6-(morpholine- |
| | 4-carbonyl)quinolin-2-yl)methylene)-1-acetylindolin-3-one |
| 113 | (Z)-N-(2-((1-acetyl-3-oxoindolin-2-ylidene)methyl)-6-(morpholine-4- |
| | carbonyl)quinolin-4-yl)acetamide |
| 114 | (Z)-1-acetyl-2-((6-(4-phenylpiperidine-1-carbonyl)quinolin-2- |
| | yl)methylene)indolin-3-one |
| 115 | (Z)-1-acetyl-2-((6-(4-morpholinopiperidine-1-carbonyl)quinolin-2- |
| | yl)methylene)indolin-3-one |
| 116 | (Z)-1-acetyl-2-((6-(4-(2-methoxyethyl)piperazine-1- |
| | carbonyl)quinolin-2-yl)methylene)indolin-3-one |
| 117 | (E)-1-acetyl-2-((6-(morpholine-4-carbonyl)-[4,4′-biquinolin]-2- |
| | yl)methylene)indolin-3-one |
| 118 | 1-acetyl-2-((6-(morpholine-4-carbonyl)-4-(naphthalen-1-yl)quinolin- |
| | 2-yl)methylene)indolin-3-one |
| 119 | (Z)-1-acetyl-2-((6-(4-((3-methoxyoxetan-3-yl)methyl)piperazine-1- |
| | carbonyl)quinolin-2-yl)methylene)indolin-3-one |
| 120 | (3-methoxyoxetan-3-yl)methyl (Z)-4-(2-((1-acetyl-3-oxoindolin-2- |
| | ylidene)methyl)quinoline-6-carbonyl)piperazine-1-carboxylate |
| 122 | 2-(3-methoxy-4-(2-morpholino-2-oxoethoxy)benzylidene)-1- |
| | methylindolin-3-one |
| 123 | ethyl 2-(3-methoxy-4-(2-morpholino-2-oxoethoxy)benzylidene)-3- |
| | oxoindoline-1-carboxylate |
| 124 | 2-(3-methoxy-4-(2-morpholino-2-oxoethoxy)benzylidene)-1- |
| | (methylsulfonyl)indolin-3-one |
| 125 | 1-acetyl-2-(4-(2-hydroxyethoxy)-3-methoxybenzylidene)indolin-3- |
| | one |
| 126 | (Z)-1-acetyl-2-((6-(2-hydroxyethoxy) quinolin-2- |
| | yl)methylene)indolin-3-one |
| 127 | (Z)-2-((1-acetyl-3-oxoindolin-2-ylidene)methyl)quinoline-6- |
| | carboxylic acid |
| 128 | (Z)-4-([1,1′-biphenyl]-2-yl)-2-((1-acetyl-3-oxoindolin-2- |
| | ylidene)methyl)quinoline-6-carboxylic acid |
| 129 | (Z)-1-(2-((1-acetyl-3-oxoindolin-2-ylidene)methyl)quinoline-6- |
| | carbonyl)piperidine-4-carboxylic acid |
| 130 | (E)-1-acetyl-2-((5-(((tetrahydro-2H-pyran-4- |
| | yl)amino)methyl)benzo[d]thiazol-2-yl)methylene)indolin-3-one |
| 131 | (Z)-1-acetyl-2-((6-(((tetrahydro-2H-pyran-4- |
| | yl)amino)methyl)quinolin-2-yl)methylene)indolin-3-one |
| 132 | (Z)-1-acetyl-2-((5-(((tetrahydro-2H-pyran-4- |
| | yl)amino)methyl)benzo[d]thiazol-2-yl)methylene)indolin-3-one |
| 133 | (Z)-1-acetyl-2-((6-(((1,1-dioxidotetrahydro-2H-thiopyran-4- |
| | yl)amino)methyl)quinolin-2-yl)methylene)indolin-3-one |
| 134 | (Z)-1-acetyl-2-((6-(((tetrahydro-2H-pyran-4- |
| | yl)amino)methyl)benzo[d]thiazol-2-yl)methylene)indolin-3-one |
| 135 | (E)-1-acetyl-2-((6-(((tetrahydro-2H-pyran-4- |
| | yl)amino)methyl)benzo[d]thiazol-2-yl)methylene)indolin-3-one |
| 136 | (Z)-1-acetyl-2-((6-((((2SR,6RS)-2,6-dimethyltetrahydro-2H-pyran-4- |
| | yl)amino)methyl)quinolin-2-yl)methylene)indolin-3-one |
| 137 | (Z)-1-acetyl-2-((6-((((2SR,6SR)-2,6-dimethyltetrahydro-2H-pyran-4- |
| | yl)amino)methyl)quinolin-2-yl)methylene)indolin-3-one |
| 138 | (Z)-1-acetyl-2-((6-(((1-(oxetan-3-yl)piperidin-4- |
| | yl)amino)methyl)quinolin-2-yl)methylene)indolin-3-one |
| 139 | 1-acetyl-2-((6-(piperazine-1-carbonyl)quinolin-2- |
| | yl)methylene)indolin-3-one dihydrochloride |
| 140 | (Z)-2-((4-([1,1′-biphenyl]-2-yl)-6-(4-methylpiperazine-1- |
| | carbonyl)quinolin-2-yl)methylene)-1-acetylindolin-3-one |
| 141 | (Z)-4-([1,1′-biphenyl]-2-yl)-2-((1-acetyl-3-oxoindolin-2- |
| | ylidene)methyl)quinoline-6-carboxamide |
| 142 | (Z)-1-(4-([1,1′-biphenyl]-2-yl)-2-((1-acetyl-3-oxoindolin-2- |
| | ylidene)methyl)quinoline-6-carbonyl)piperidine-4-carboxylic acid |
| 143 | (Z)-2-((4-([1,1′-biphenyl]-2-yl)-6-(((tetrahydro-2H-pyran-4- |
| | yl)amino)methyl)quinolin-2-yl)methylene)-1-acetylindolin-3-one |
| 144 | (Z)-1-acetyl-2-((4-(2-acetyl-1,2,3,4-tetrahydroisoquinolin-6-yl)-6- |
| | (morpholine-4-carbonyl)quinolin-2-yl)methylene)indolin-3-one |
| 145 | 2-((4-(1H-indazol-4-yl)-6-(morpholine-4-carbonyl)quinolin-2- |
| | yl)methylene)-1-acetylindolin-3-one |
| 146 | 1-acetyl-2-((4-(2-acetyl-1,2,3,4-tetrahydroisoquinolin-5-yl)-6- |
| | (morpholine-4-carbonyl)quinolin-2-yl)methylene)indolin-3-one |
| 147 | 6-(2-((1-acetyl-3-oxoindolin-2-ylidene)methyl)-6-(morpholine-4- |
| | carbonyl)quinolin-4-yl)-3,4-dihydroisoquinolin-1(2H)-one |
| 148 | 2-((4-(3-(1H-pyrazol-4-yl)phenyl)-6-(morpholine-4- |
| | carbonyl)quinolin-2-yl)methylene)-1-acetylindolin-3-one |
| 149 | 1-acetyl-2-((4-(3-(1-methyl-1H-pyrazol-4-yl)phenyl)-6-(morpholine- |
| | 4-carbonyl)quinolin-2-yl)methylene)indolin-3-one |
| 150 | 2-((4-([1,1′-biphenyl]-4-yl)-6-(morpholine-4-carbonyl)quinolin-2- |
| | yl)methylene)-1-acetylindolin-3-one |
| 151 | 1-acetyl-2-((6-(morpholine-4-carbonyl)-4-(naphthalen-2-yl)quinolin- |
| | 2-yl)methylene)indolin-3-one |
| 152 | 4-([1,1′-biphenyl]-3-yl)-2-((1-acetyl-3-oxoindolin-2- |
| | ylidene)methyl)quinoline-6-carboxylic acid |
| 153 | 2-((1-acetyl-3-oxoindolin-2-ylidene)methyl)-4-(naphthalen-1- |
| | yl)quinoline-6-carboxylic acid |
| 154 | 2-((1-acetyl-3-oxoindolin-2-ylidene)methyl)-[4,4′-biquinoline]-6- |
| | carboxylic acid |
| 155 | 2-((1-acetyl-3-oxoindolin-2-ylidene)methyl)-[4,8′-biquinoline]-6- |
| | carboxylic acid |
| 156 | 4-([1,1′-biphenyl]-4-yl)-2-((1-acetyl-3-oxoindolin-2- |
| | ylidene)methyl)quinoline-6-carboxylic acid |
| 157 | 2-((1-acetyl-3-oxoindolin-2-ylidene)methyl)-4-(naphthalen-2- |
| | yl)quinoline-6-carboxylic acid |
| 158 | 2-((4-([1,1′-biphenyl]-3-yl)-6-(4-(oxetan-3-yl)piperazine-1- |
| | carbonyl)quinolin-2-yl)methylene)-1-acetylindolin-3-one |
| 159 | 1-acetyl-2-((4-(naphthalen-1-yl)-6-(4-(oxetan-3-yl)piperazine-1- |
| | carbonyl)quinolin-2-yl)methylene)indolin-3-one |
| 160 | 1-acetyl-2-((6-(4-(oxetan-3-yl)piperazine-1-carbonyl)-[4,4′- |
| | biquinolin]-2-yl)methylene)indolin-3-one |
| 161 | 1-acetyl-2-((6-(4-(oxetan-3-yl)piperazine-1-carbonyl)-[4,8′- |
| | biquinolin]-2-yl)methylene)indolin-3-one |
| 162 | 2-((4-([1,1′-biphenyl]-4-yl)-6-(4-(oxetan-3-yl)piperazine-1- |
| | carbonyl)quinolin-2-yl)methylene)-1-acetylindolin-3-one |
| 163 | 1-acetyl-2-((4-(naphthalen-2-yl)-6-(4-(oxetan-3-yl)piperazine-1- |
| | carbonyl)quinolin-2-yl)methylene)indolin-3-one |
| 164 | 2-((4-([1,1′-biphenyl]-3-yl)-6-(((tetrahydro-2H-pyran-4- |
| | yl)amino)methyl)quinolin-2-yl)methylene)-1-acetylindolin-3-one |
| 165 | 1-acetyl-2-((4-(naphthalen-1-yl)-6-(((tetrahydro-2H-pyran-4- |
| | yl)amino)methyl)quinolin-2-yl)methylene)indolin-3-one |
| 166 | 1-acetyl-2-((6-(((tetrahydro-2H-pyran-4-yl)amino)methyl)-[4,4′- |
| | biquinolin]-2-yl)methylene)indolin-3-one |
| 167 | 1-acetyl-2-((6-(((tetrahydro-2H-pyran-4-yl)amino)methyl)-[4,8′- |
| | biquinolin]-2-yl)methylene)indolin-3-one |
| 168 | 2-((4-([1,1′-biphenyl]-4-yl)-6-(((tetrahydro-2H-pyran-4- |
| | yl)amino)methyl)quinolin-2-yl)methylene)-1-acetylindolin-3-one |
| 169 | 1-acetyl-2-((4-(naphthalen-2-yl)-6-(((tetrahydro-2H-pyran-4- |
| | yl)amino)methyl)quinolin-2-yl)methylene)indolin-3-one |
| 170 | 1-acetyl-2-((6-(morpholine-4-carbonyl)-4-(1H-pyrazol-4-yl)quinolin- |
| | 2-yl)methylene)indolin-3-one |
| 171 | 1-acetyl-2-((4-(1-methyl-1H-pyrazol-4-yl)-6-(morpholine-4- |
| | carbonyl)quinolin-2-yl)methylene)indolin-3-one |
| 172 | 1-acetyl-2-((6-(morpholine-4-carbonyl)-3-(naphthalen-1-yl)quinolin- |
| | 2-yl)methylene)indolin-3-one |
| 173 | 1-acetyl-2-((4-(2-methyl-1,2,3,4-tetrahydroisoquinolin-6-yl)-6- |
| | (morpholine-4-carbonyl)quinolin-2-yl)methylene)indolin-3-one |
| 174 | 4-(2-((1-acetyl-3-oxoindolin-2-ylidene)methyl)-6-(morpholine-4- |
| | carbonyl)quinolin-4-yl)benzoic acid |
| 175 | 3-(2-((1-acetyl-3-oxoindolin-2-ylidene)methyl)-6-(morpholine-4- |
| | carbonyl)quinolin-4-yl)benzoic acid |
| 176 | 2-(2-((1-acetyl-3-oxoindolin-2-ylidene)methyl)-6-(morpholine-4- |
| | carbonyl)quinolin-4-yl)benzoic acid |
| 177 | (Z)-1-acetyl-2-((4-(1-methyl-1H-pyrazol-4-yl)quinolin-2- |
| | yl)methylene)indolin-3-one |
| 178 | (Z)-1-acetyl-2-((4-(1-ethyl-1H-pyrazol-4-yl)-6-(morpholine-4- |
| | carbonyl)quinolin-2-yl)methylene)indolin-3-one |
| 179 | (Z)-1-acetyl-2-((4-(1-acetylpiperidin-3-yl)-6-(morpholine-4- |
| | carbonyl)quinolin-2-yl)methylene)indolin-3-one |
| 180 | (Z)-1-acetyl-2-((4-(1-methyl-1H-imidazol-2-yl)-6-(morpholine-4- |
| | carbonyl)quinolin-2-yl)methylene)indolin-3-one |
| 181 | (Z)-1-acetyl-2-((4-(morpholinomethyl)quinolin-2- |
| | yl)methylene)indolin-3-one |
| 182 | (Z)-1-acetyl-2-((4-(2-acetyl-1,2,3,4-tetrahydroisoquinolin-7-yl)-6- |
| | (morpholine-4-carbonyl)quinolin-2-yl)methylene)indolin-3-one |
| 183 | 4-((2-((1-acetyl-3-oxoindolin-2-ylidene)methyl)quinolin-4- |
| | yl)methyl)morpholin-3-one |
| 184 | 1-acetyl-2-((6-(morpholine-4-carbonyl)-4-(pyridin-4-yl)quinolin-2- |
| | yl)methylene)indolin-3-one |
| 185 | 1-acetyl-2-((6-(morpholine-4-carbonyl)-4-(pyrimidin-5-yl)quinolin-2- |
| | yl)methylene)indolin-3-one |
| 186 | 1-acetyl-2-((4-(morpholine-4-carbonyl)quinolin-2- |
| | yl)methylene)indolin-3-one |
| 187 | (Z)-1-acetyl-2-((4-(2-acetyl-1,2,3,4-tetrahydroisoquinolin-6- |
| | yl)quinolin-2-yl)methylene)indolin-3-one |
| 188 | (Z)-1-acetyl-2-((4-(1-methyl-1H-1,2,4-triazol-3-yl)-6-(morpholine-4- |
| | carbonyl)quinolin-2-yl)methylene)indolin-3-one |
| 189 | (Z)-1-acetyl-2-((6-(morpholine-4-carbonyl)-4-(pyrimidin-2- |
| | yl)quinolin-2-yl)methylene)indolin-3-one |
| 190 | (Z)-6-(2-((1-acetyl-3-oxoindolin-2-ylidene)methyl)quinolin-4-yl)-3,4- |
| | dihydroisoquinolin-1(2H)-one |
| 191 | (Z)-1-acetyl-2-((6-(morpholine-4-carbonyl)-4-(pyridazin-3- |
| | yl)quinolin-2-yl)methylene)indolin-3-one |
| 192 | (Z)-1-acetyl-2-((4-(1-methyl-1H-imidazol-4-yl)-6-(morpholine-4- |
| | carbonyl)quinolin-2-yl)methylene)indolin-3-one |
| 193 | (Z)-1-acetyl-2-((4-(2-acetyl-1,2,3,4-tetrahydroisoquinolin-5- |
| | yl)quinolin-2-yl)methylene)indolin-3-one |
| 194 | (Z)-1-acetyl-2-((6-(morpholine-4-carbonyl)-4-(pyrazin-2-yl)quinolin- |
| | 2-yl)methylene)indolin-3-one |
| 195 | (Z)-1-acetyl-2-((6-(morpholine-4-carbonyl)-5-(naphthalen-1- |
| | yl)quinolin-2-yl)methylene)indolin-3-one |
| 196 | (Z)-1-acetyl-2-((4-(2-(1-methyl-1H-pyrazol-4-yl)phenyl)-6- |
| | (morpholine-4-carbonyl)quinolin-2-yl)methylene)indolin-3-one |
| 197 | 1-acetyl-2-((6-(morpholine-4-carbonyl)-8-(naphthalen-1-yl)quinolin- |
| | 2-yl)methylene)indolin-3-one |
| 198 | (Z)-1-acetyl-2-((3-(1-methyl-1H-pyrazol-4-yl)-6-(morpholine-4- |
| | carbonyl)quinolin-2-yl)methylene)indolin-3-one |
| 199 | (Z)-1-acetyl-2-((5-(1-methyl-1H-pyrazol-4-yl)-6-(morpholine-4- |
| | carbonyl)quinolin-2-yl)methylene)indolin-3-one |
| 200 | (Z)-1-acetyl-2-((5-(2-acetyl-1,2,3,4-tetrahydroisoquinolin-6-yl)-6- |
| | (morpholine-4-carbonyl)quinolin-2-yl)methylene)indolin-3-one |
| 201 | (Z)-1-acetyl-2-((5-(2-acetyl-1,2,3,4-tetrahydroisoquinolin-7-yl)-6- |
| | (morpholine-4-carbonyl)quinolin-2-yl)methylene)indolin-3-one |
| 202 | (Z)-2-((1-acetyl-3-oxoindolin-2-ylidene)methyl)-3-(naphthalen-1- |
| | yl)quinoline-6-carboxylic acid |
| 203 | (Z)-1-acetyl-2-((4-(1-methyl-1H-pyrazol-4-yl)quinazolin-2- |
| | yl)methylene)indolin-3-one |
| 204 | 1-acetyl-2-((4-(4-(oxetan-3-yl)piperazine-1-carbonyl)quinolin-2- |
| | yl)methylene)indolin-3-one |
| 205 | (Z)-1-acetyl-2-((4-(1-methyl-1H-pyrazol-4-yl)-6-(((tetrahydro-2H- |
| | pyran-4-yl)amino)methyl)quinolin-2-yl)methylene)indolin-3-one |
| 206 | 1-acetyl-2-((4-(2-(1-methyl-1H-pyrazol-4-yl)phenyl)-6-(((tetrahydro- |
| | 2H-pyran-4-yl)amino)methyl)quinolin-2-yl)methylene)indolin-3-one |
| 207 | 1-acetyl-2-((4-(2-acetyl-1,2,3,4-tetrahydroisoquinolin-5-yl)-6- |
| | (((tetrahydro-2H-pyran-4-yl)amino)methyl)quinolin-2- |
| | yl)methylene)indolin-3-one |
| 208 | (Z)-1-acetyl-2-((4-(((tetrahydro-2H-pyran-4- |
| | yl)amino)methyl)quinolin-2-yl)methylene)indolin-3-one |
| 209 | (Z)-1-acetyl-2-((4-(2-acetyl-1,2,3,4-tetrahydroisoquinolin-6-yl)-6- |
| | (((tetrahydro-2H-pyran-4-yl)amino)methyl)quinolin-2- |
| | yl)methylene)indolin-3-one |
| 210 | (Z)-1-acetyl-2-((4-(3-(1-methyl-1H-pyrazol-4-yl)phenyl)-6- |
| | (((tetrahydro-2H-pyran-4-yl)amino)methyl)quinolin-2- |
| | yl)methylene)indolin-3-one |
| 211 | (Z)-1-acetyl-2-((4-(2-acetyl-1,2,3,4-tetrahydroisoquinolin-7-yl)-6- |
| | (((tetrahydro-2H-pyran-4-yl)amino)methyl)quinolin-2- |
| | yl)methylene)indolin-3-one |
| 212 | 1-acetyl-2-((6-(morpholine-4-carbonyl)-4-(1H-1,2,4-triazol-3- |
| | yl)quinolin-2-yl)methylene)indolin-3-one |
| 213 | (Z)-2-((4-(1H-pyrazol-4-yl)quinolin-2-yl)methylene)-1-acetylindolin- |
| | 3-one |
| 214 | (Z)-1-acetyl-2-((3-(naphthalen-1-yl)-6-(1H-tetrazol-5-yl)quinolin-2- |
| | yl)methylene)indolin-3-one |

(23) As a more preferred aspect, the compound (I) of the present invention is a compound selected from the compounds shown below:

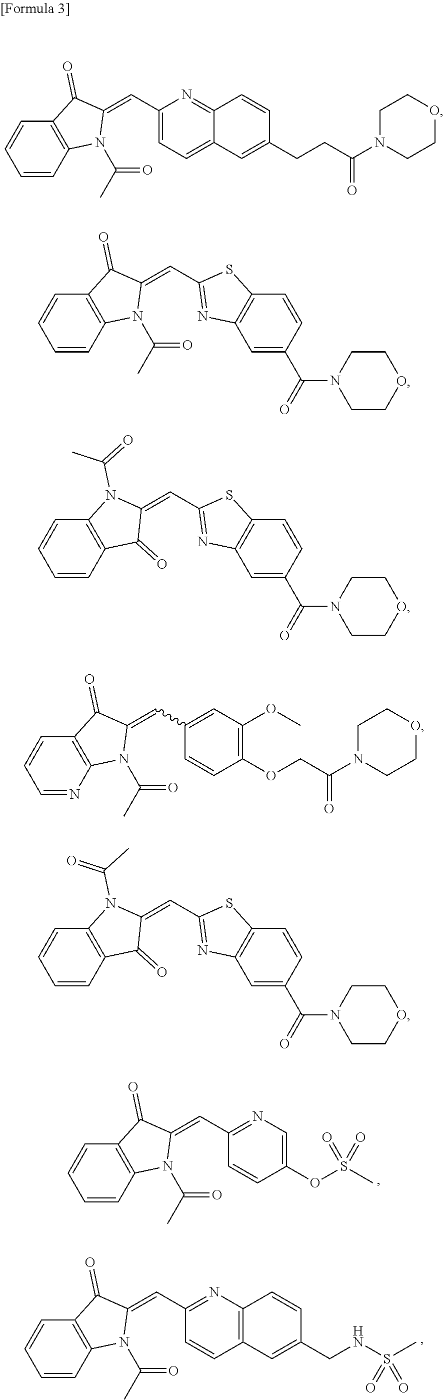

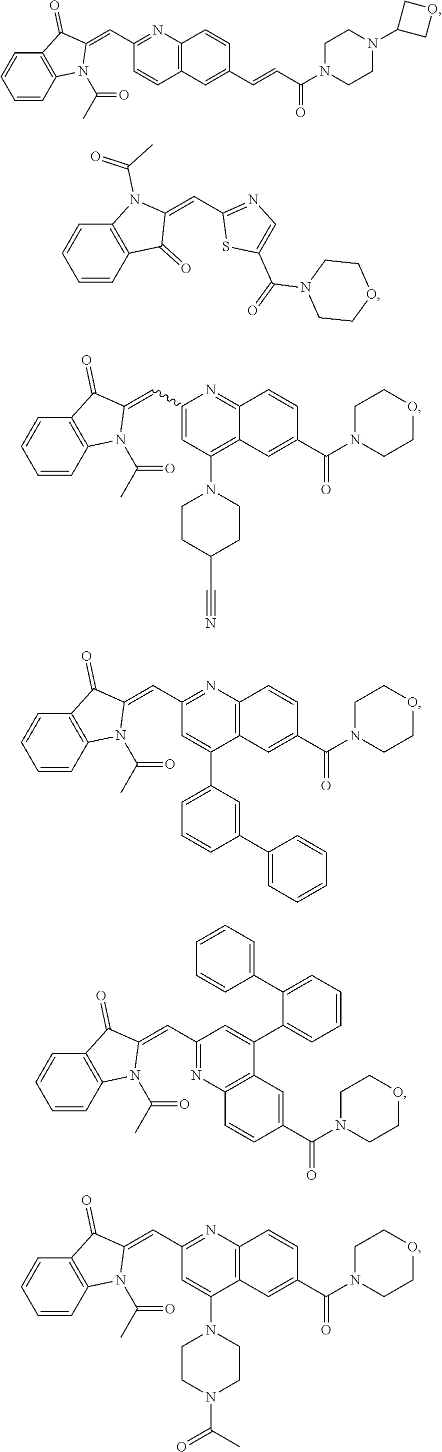

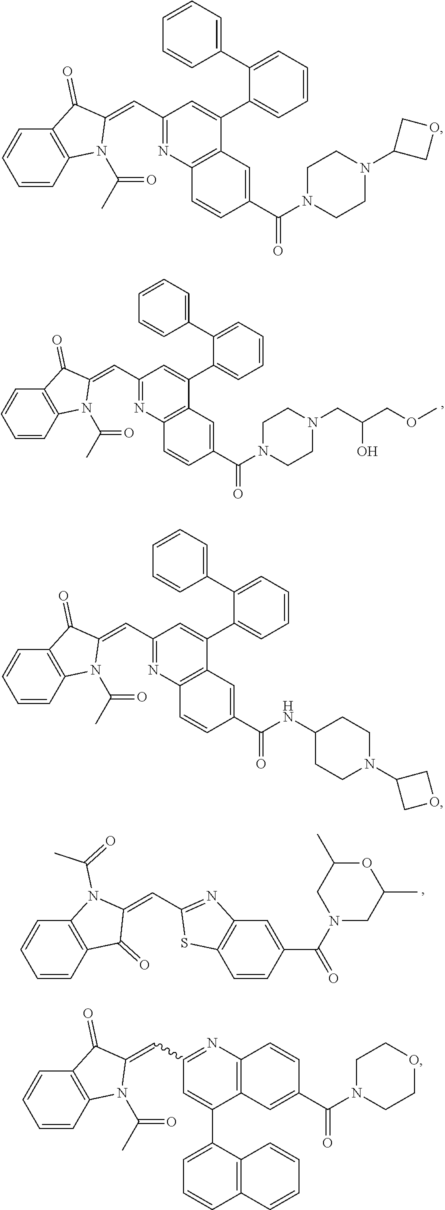

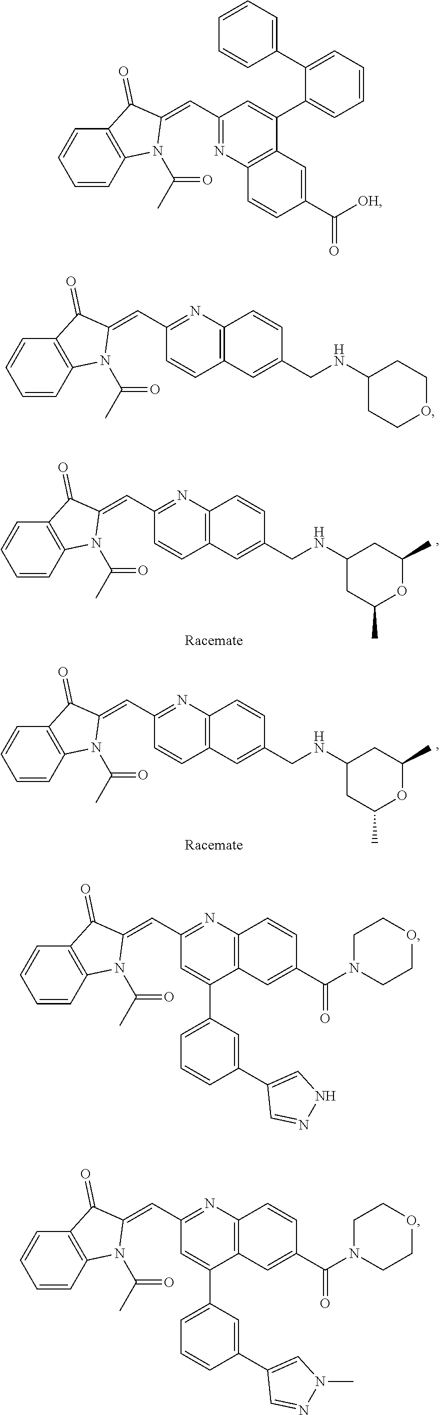 [SMILES: CC(=O)N1/C(=C\c2cc(-c3ccccc3-c3ccccc3)c3cc(C(=O)O)ccc3n2)C(=O)c2ccccc21 ; CC(=O)N1/C(=C\c2ccc3cc(CNC4CCOCC4)ccc3n2)C(=O)c2ccccc21 ; CC(=O)N1/C(=C\c2ccc3cc(CNC4C[C@@H](C)O[C@@H](C)C4)ccc3n2)C(=O)c2ccccc21 ; CC(=O)N1/C(=C\c2ccc3cc(CNC4C[C@@H](C)O[C@H](C)C4)ccc3n2)C(=O)c2ccccc21 ; CC(=O)N1/C(=C\c2cc(-c3cccc(-c4cn[nH]c4)c3)c3cc(C(=O)N4CCOCC4)ccc3n2)C(=O)c2ccccc21 ; CC(=O)N1/C(=C\c2cc(-c3cccc(-c4cnn(C)c4)c3)c3cc(C(=O)N4CCOCC4)ccc3n2)C(=O)c2ccccc21]

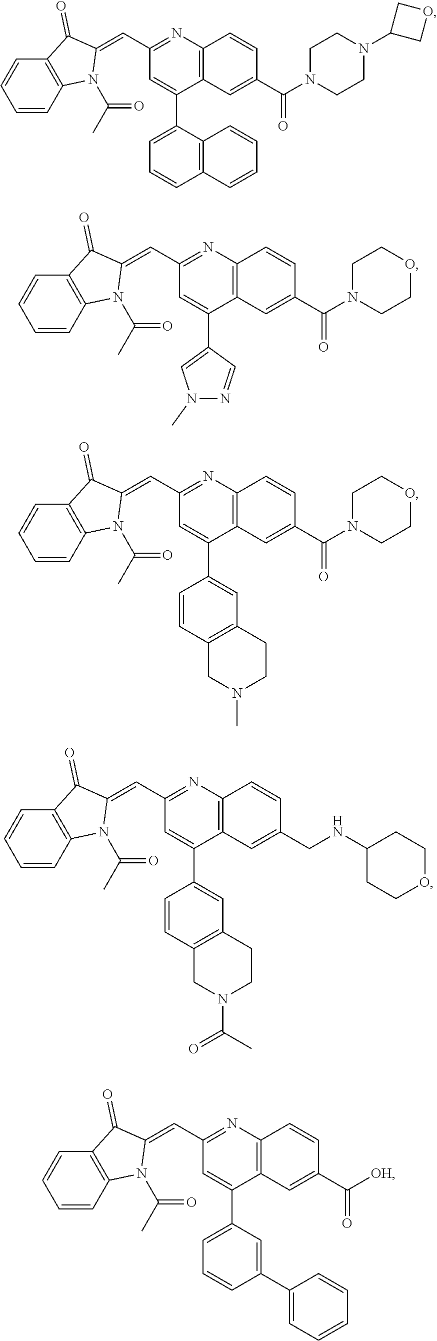 [SMILES: CC(=O)N1/C(=C\c2cc(-c3cccc4ccccc34)c3cc(C(=O)N4CCN(C5COC5)CC4)ccc3n2)C(=O)c2ccccc21 ; CC(=O)N1/C(=C\c2cc(-c3cnn(C)c3)c3cc(C(=O)N4CCOCC4)ccc3n2)C(=O)c2ccccc21 ; CC(=O)N1/C(=C\c2cc(-c3ccc4c(c3)CCN(C)C4)c3cc(C(=O)N4CCOCC4)ccc3n2)C(=O)c2ccccc21 ; CC(=O)N1CCc2cc(-c3cc(/C=C4/C(=O)c5ccccc5N4C(C)=O)nc4ccc(CNC5CCOCC5)cc34)ccc2C1 ; CC(=O)N1/C(=C\c2cc(-c3cccc(-c4ccccc4)c3)c3cc(C(=O)O)ccc3n2)C(=O)c2ccccc21]

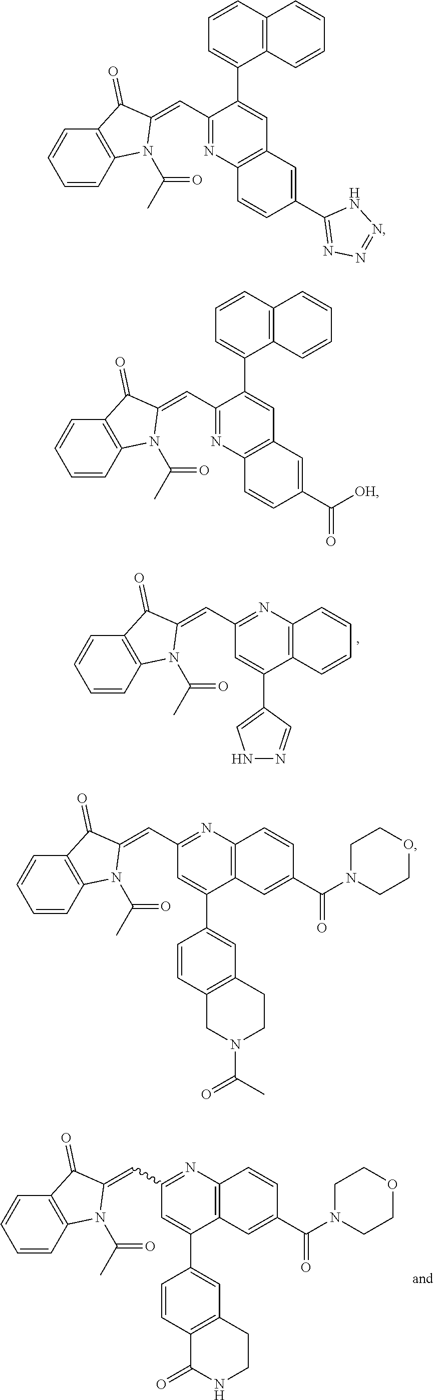 [SMILES: CC(=O)N1/C(=C\c2nc3ccc(-c4nnn[nH]4)cc3cc2-c2cccc3ccccc23)C(=O)c2ccccc21 ; CC(=O)N1/C(=C\c2nc3ccc(C(=O)O)cc3cc2-c2cccc3ccccc23)C(=O)c2ccccc21 ; CC(=O)N1/C(=C\c2cc(-c3cn[nH]c3)c3ccccc3n2)C(=O)c2ccccc21 ; CC(=O)N1CCc2cc(-c3cc(/C=C4/C(=O)c5ccccc5N4C(C)=O)nc4ccc(C(=O)N5CCOCC5)cc34)ccc2C1 ; CC(=O)N1C(=Cc2cc(-c3ccc4c(c3)CCNC4=O)c3cc(C(=O)N4CCOCC4)ccc3n2)C(=O)c2ccccc21]

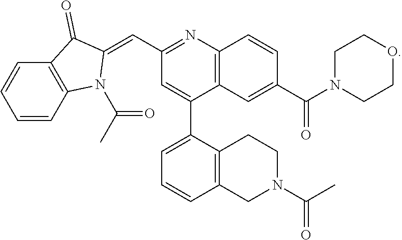 [SMILES: CC(=O)N1CCc2c(cccc2-c2cc(/C=C3/C(=O)c4ccccc4N3C(C)=O)nc3ccc(C(=O)N4CCOCC4)cc23)C1]

(24) As a preferred aspect of the present invention, it is the compound represented by the formula (I) according to any one of the above (1) to (23) or a pharmaceutically acceptable salt thereof, wherein the wavy line portion of the compound represented by the formula (I) is a Z-form.

(25) As a preferred aspect of the present invention, it is the compound represented by the formula (I) according to any one of the above (1) to (23) or a pharmaceutically acceptable salt thereof, wherein the wavy line portion of the compound represented by the formula (I) is an E-form.

(26) As another aspect of the present invention, a Ras/Raf binding inhibitor, comprising the compound represented by the formula (I) or a pharmaceutically acceptable salt thereof, or an isomer thereof, is provided:

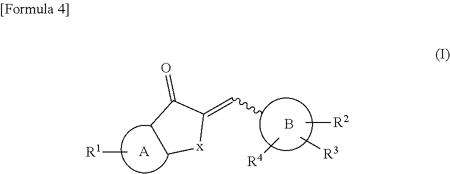

wherein
A represents a benzene ring or a pyridine ring,
B represents a C₆₋₁₀ aryl group, or a heteroaryl group containing 1 to 4 atoms selected from N, S and O,
X represents —NR⁵—,
R¹ represents H, a C₁₋₆ alkyl group optionally substituted with a NHCOCH₃ group, or halogen,
R², R³ and R⁴, which are the same or different, each represent (provided that at least any one of them is not H)
H;
C₁₋₆ alkyl-SO₂— (e.g. mesyl, etc.);
cyano;
halogen;
nitro;
azide;
—CO—R¹¹ group (wherein the R¹¹ represents
OH,
an R¹² group (wherein the R¹² group represents a heterocyclyl group containing 1 or 2 atoms (groups) selected from N, S, SO, SO₂ and O, and optionally having a substituent),
NH₂, NHR¹², or N(R¹²)R¹², or
C₆₋₁₀ arylamino);
an —O—CH₂—CO—R¹¹ group;
a C₁₋₆ alkyl group optionally having a substituent
(wherein the substituent represents
   —CONH₂,
   an R¹² group,
   OH,
   —NR¹³R¹⁴ (wherein the R¹³ represents C₁₋₆ alkyl-SO₂— (e.g. mesyl, etc.), C₁₋₆ alkyl-CO— (preferably, acetyl), or an R¹² group, and the R¹⁴ represents H, a C₁₋₆ alkyl group, or an R¹² group),
   R¹²—CO—, or
   R¹²—C₁₋₆ alkyl-CONH—);
   an —OR¹⁵ group
(wherein the R¹⁵ represents
   H,
   a C₁₋₆ alkyl group optionally having a substituent (wherein the substituent represents OH, C₁₋₆ alkoxy, C₆₋₁₀ aryl, C₆₋₁₀ aryloxy, R¹², cyano, C₁₋₆ alkyl-SO₂— (e.g. mesyl, etc.), or methylsulfinyl),
   C₁₋₆ alkyl-SO₂— (e.g. mesyl, etc.), or
   a heteroaryl group containing 1 to 4 atoms selected from N, S and O);
   an —NR¹⁶R¹⁷ group
(wherein the R¹⁶ and R¹⁷, which are the same or different, each represent
   H,
   C₁₋₆ alkyl-CO—,
   an R¹² group, or
   R¹²—C₁₋₆ alkyl-CO—,
or
R¹⁶ and R¹⁷ may form an R¹² group, together with N);
   a C₆₋₁₀ aryl group optionally having a substituent
(wherein the substituent represents C₆₋₁₀ aryloxy, a heteroaryl group containing 1 to 4 atoms selected from N, S and O, and optionally having a substituent, or phenyl optionally annelated with R¹²);
   a heteroaryl group containing 1 to 4 atoms selected from N, S and O, and optionally having a substituent; or
   —(CH═CH)—R¹⁸ (wherein the R¹⁸ represents —CO—R¹⁹ or —SO₂—R¹⁹, and the R¹⁹ represents NH₂, or an R¹² group); wherein
      R⁵ represents C₁₋₆ alkyl, —COR⁶, —COOR⁶, —CONR⁶R⁷, —SOnR⁶, or SOnNR⁶R⁷, wherein n represents 0, 1 or 2, and
      R⁶ and R⁷, which are the same or different, each represent H, a C₁₋₆ alkyl group, or a C₆₋₁₀ aryl group, and
      the wavy line represents a geometrical isomer.

(27) As another preferred aspect of the present invention, it is a Ras/Raf binding inhibitor, comprising the compound represented by the formula (I) according to the above (26) or a pharmaceutically acceptable salt thereof, or an isomer thereof, wherein A represents a benzene ring.

(28) As another preferred aspect of the present invention, it is a Ras/Raf binding inhibitor, comprising the compound represented by the formula (I) according to the above (26) or (27) or a pharmaceutically acceptable salt thereof, or an isomer thereof, wherein B represents phenyl, or a heteroaryl group containing 1 to 4 atoms selected from N, S and O.

(29) As another more preferred aspect of the present invention, it is a Ras/Raf binding inhibitor, comprising the compound represented by the formula (I) according to the above (26) or (27) or a pharmaceutically acceptable salt thereof, or an isomer thereof, wherein B represents phenyl, pyridyl, quinolyl, indolyl, thiazolyl, pyrrolopyridinyl, benzothiazolyl, or furopyridinyl.

(30) As another more preferred aspect of the present invention, it is a Ras/Raf binding inhibitor, comprising the compound represented by the formula (I) according to the above (26) or (27) or a pharmaceutically acceptable salt thereof, or an isomer thereof, wherein B represents the following:

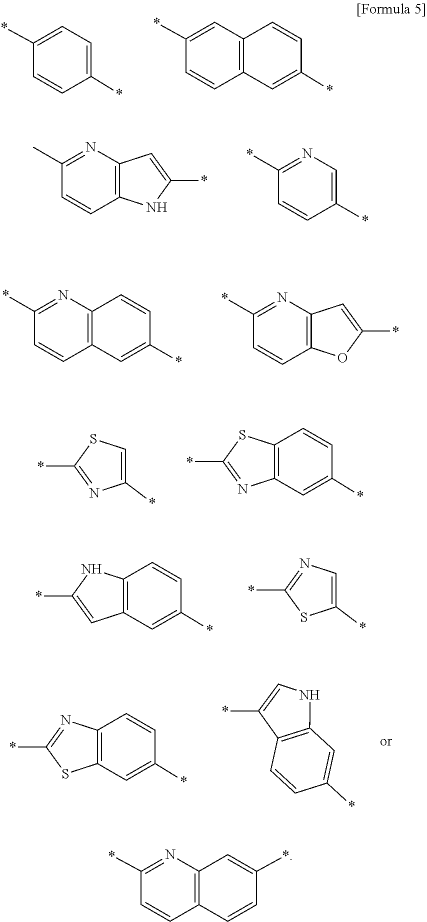

In the above formula, one of the bonding hands corresponds to the bonding of the wavy line in the formula (I), and the other bonding hand represents a bonding hand with any one substituent other than the hydrogen in R², R³ and R⁴.

Besides, in such a case, if there is another substituent, the substituent is substituted with the residual position described above.

(31) As another more preferred aspect of the present invention, it is a Ras/Raf binding inhibitor, comprising the compound represented by the formula (I) according to the above (26) or (27) or a pharmaceutically acceptable salt thereof, or an isomer thereof, wherein B represents phenyl, quinolyl, thiazolyl, or benzothiazolyl.

(32) As another preferred aspect of the present invention, it is a Ras/Raf binding inhibitor, comprising the compound represented by the formula (I) according to any one of the above (26) to (31) or a pharmaceutically acceptable salt thereof, or an isomer thereof, wherein the R¹ represents H.

(33) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the R¹¹ of the —CO—R¹¹ group as represented in R², R³ and R⁴ represents OH or an R¹² group.

(34) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the R¹¹ of the —O—CH₂—CO—R¹¹ group as represented in R², R³ and R⁴ represents an R¹² group.

(35) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the substituent of the C₁₋₆ alkyl group optionally having a substituent, as represented in R², R³ and R⁴, represents an R¹² group, OH, —NR¹³R¹⁴ (wherein the R¹³ represents C₁₋₆ alkyl-SO₂— (e.g. mesyl, etc.) or an R¹² group, and the R¹⁴ represents H or a C₁₋₆ alkyl group), R¹²—CO—, or R¹²—C₁₋₆ alkyl-CONH—.

(36) As another more preferred aspect of the present invention, it is a Ras/Raf binding inhibitor, comprising the compound represented by the formula (I) according to the above (35) or a pharmaceutically acceptable salt thereof, or an isomer thereof, wherein the substituent of the C₁₋₆ alkyl group optionally having a substituent, as represented in R², R³ and R⁴, represents —NR¹³R¹⁴ (wherein the R¹³ represents C₁₋₆ alkyl-SO₂— (e.g. mesyl, etc.), and the R¹⁴ represents H).

(37) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the R¹⁵ of the —OR¹⁵ group as represented in R², R³ and R⁴ represents a C₁₋₆ alkyl group (preferably, methyl) or C₁₋₆ alkyl-SO₂— (e.g. mesyl, etc.).

(38) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the R¹⁶ and R¹⁷ of the —NR¹⁶R¹⁷ group as represented in R², R³ and R⁴, which are the same or different, each represent H or an R¹² group, or the R¹⁶ and R¹⁷ together represent an R¹² group (preferably, acetylpiperazinyl and cyanopiperidino).

(39) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the C₆₋₁₀ aryl group of the C₆₋₁₀ aryl group optionally having a substituent, as represented in R², R³ and R⁴, represents phenyl or naphthyl, and the substituent represents a heteroaryl group containing 1 to 4 atoms selected from N, S and O, and optionally having a substituent (preferably, pyridyl, phenylpyridyl, quinolyl, indazolyl, pyrazolyl, or methylpyrazolyl; more preferably, indazolyl, pyrazolyl, or methylpyrazolyl), or phenyl optionally annelated with an R¹² group (preferably, phenyl).

(40) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein the heteroaryl group containing 1 to 4 atoms selected from N, S and O, and optionally having a substituent, as represented in R², R³ and R⁴, represents pyridyl, phenylpyridyl, quinolyl, indazolyl, pyrazolyl, or methylpyrazolyl.

(41) As another more preferred aspect of the present invention, it is a Ras/Raf binding inhibitor, comprising the compound represented by the formula (I) according to the above (40) or a pharmaceutically acceptable salt thereof, or an isomer thereof, wherein the heteroaryl group containing 1 to 4 atoms selected from N, S and O, and optionally having a substituent, as represented in R², R³ and R⁴, represents indazolyl, pyrazolyl, or methylpyrazolyl.

(42) As another preferred aspect of the present invention, it is a Ras/Raf binding inhibitor, comprising the compound represented by the formula (I) according to any one of the above (26) to (32) or a pharmaceutically acceptable salt thereof, or an isomer thereof, wherein R², R³ and R⁴ represent —(CH═CH)—R¹⁸ (wherein the R¹⁸ represents —CO—R¹⁹, and the R¹⁹ represents an R¹² group).

(43) As another preferred aspect of the present invention, it is a Ras/Raf binding inhibitor, comprising the compound represented by the formula (I) according to any one of the above (27) to (42) or a pharmaceutically acceptable salt thereof, or an isomer thereof, wherein the heterocyclyl group containing 1 or 2 atoms (groups) selected from N, S, SO, SO₂ and O, in the heterocyclyl group containing 1 or 2 atoms (groups) selected from N, S, SO, SO₂ and O, and optionally having a substituent, as represented in R¹², represents morpholino, piperazinyl, thiomorpholino, dioxidothiomorpholino, tetrahydropyranyl, tetrahydrothiopyranyl, pyrrolidinyl, dioxidotetrahydrothiopyranyl, or piperidinyl.

(44) As another more preferred aspect of the present invention, it is a Ras/Raf binding inhibitor, comprising the compound represented by the formula (I) according to the above (43) or a pharmaceutically acceptable salt thereof, or an isomer thereof, wherein the heterocyclyl group containing 1 or 2 atoms (groups) selected from N, S, SO, SO₂ and O, in the heterocyclyl group containing 1 or 2 atoms (groups) selected from N, S, SO, SO₂ and O, and optionally having a substituent, as represented in R¹², represents morpholino, piperazinyl, piperidinyl, or tetrahydropyranyl.

(45) As another preferred aspect of the present invention, it is a Ras/Raf binding inhibitor, comprising the compound represented by the formula (I) according to any one of the above (26) to (44) or a pharmaceutically acceptable salt thereof, or an isomer thereof, wherein the substituent in the heterocyclyl group containing 1 or 2 atoms (groups) selected from N, S, SO, SO₂ and O, and optionally having a substituent, as represented in R¹², represents —CO—, —COOH, cyano, 1 or 2 C₁₋₆ alkyl groups (preferably, methyl groups on both sides of the heteroatom selected from N, S, SO, SO₂ and O of the heterocyclyl group, more preferably, 2,6-dimethyl for tetrahydropyranyl and morpholino), C₁₋₆ alkyl-CO— (preferably, acetyl), C₁₋₆ alkyl-SO₂— (e.g. mesyl, etc.), C₆₋₁₀ aryl (preferably, phenyl), 3-methoxy-2-hydroxypropyl, an R¹² group (preferably, oxetanyl or morpholino), R¹²—CH₂-(preferably, methoxyoxetanylmethyl), R¹²—CH₂CO— (preferably, morpholinomethylcarbonyl), R¹²—CH₂OCO— (preferably, methoxyoxetanylmethoxycarbonyl), or methoxyethyl.

(46) As another preferred aspect of the present invention, it is a Ras/Raf binding inhibitor, comprising the compound represented by the formula (I) according to any one of the above (26) to (45) or a pharmaceutically acceptable salt thereof, or an isomer thereof, wherein the R⁵ represents —COR⁶, and the R⁶ represents a C₁₋₆ alkyl group.

(47) As another preferred aspect of the present invention, it is the Ras/Raf binding inhibitor according to the above (26), wherein the compound represented by the formula (I) or a pharmaceutically acceptable salt thereof, or an isomer thereof, is a compound selected from Compounds 1 to 214 shown in Table 1.

(48) As another more preferred aspect of the present invention, it is the Ras/Raf binding inhibitor according to the above (26), wherein the compound represented by the formula (I) is a compound selected from the following compounds:

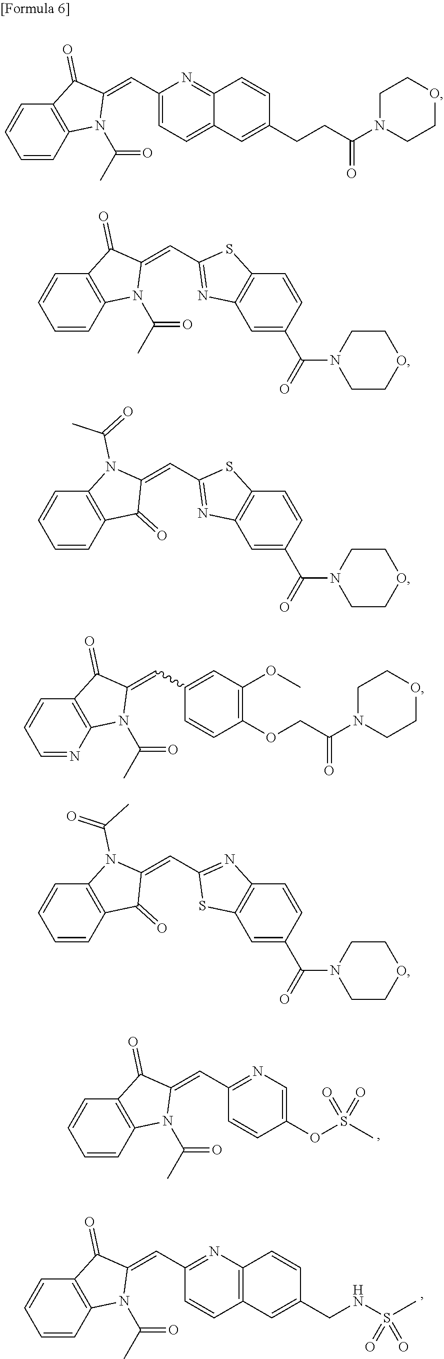

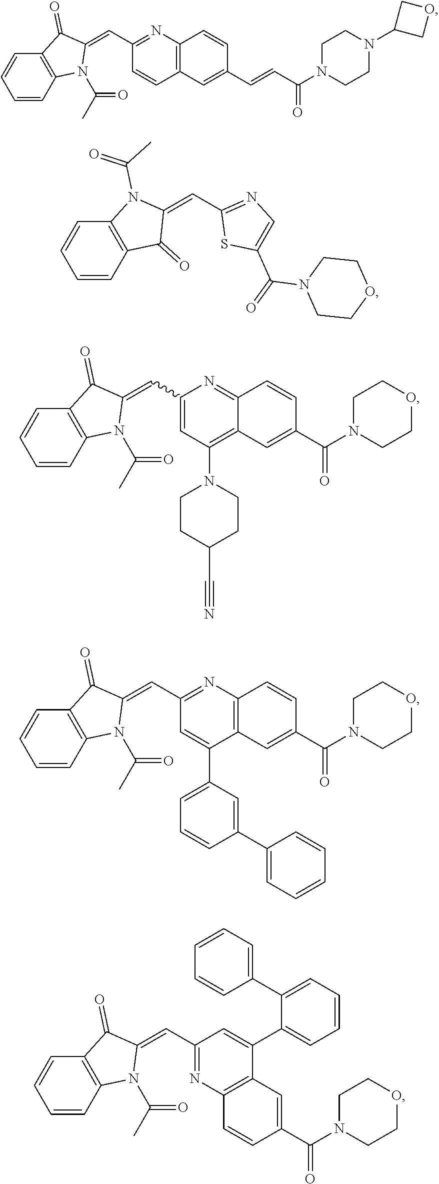

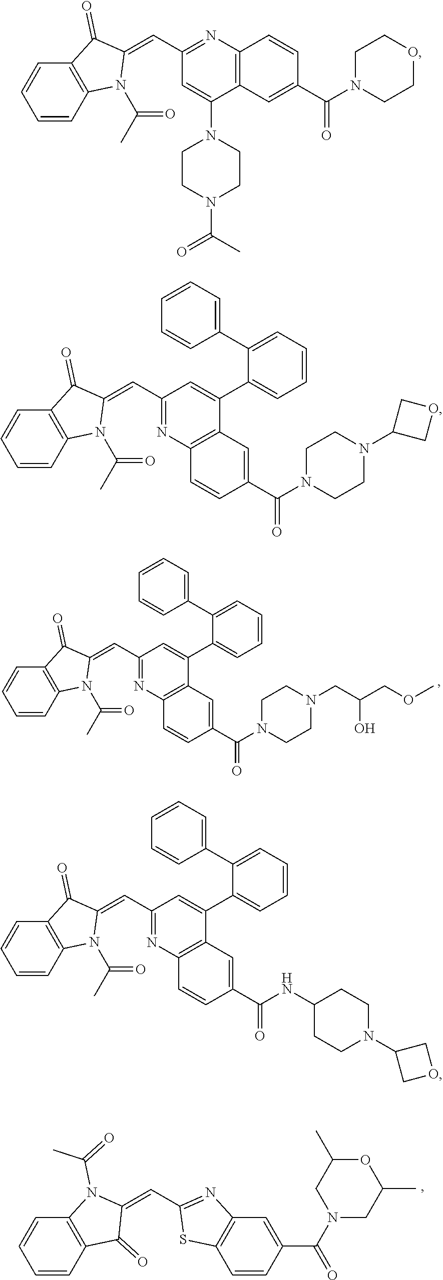

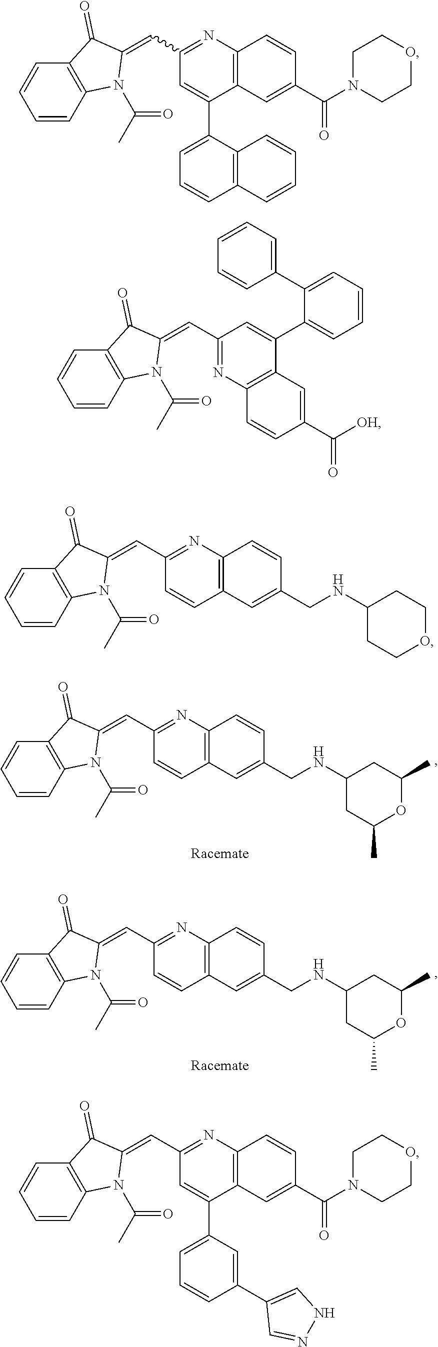

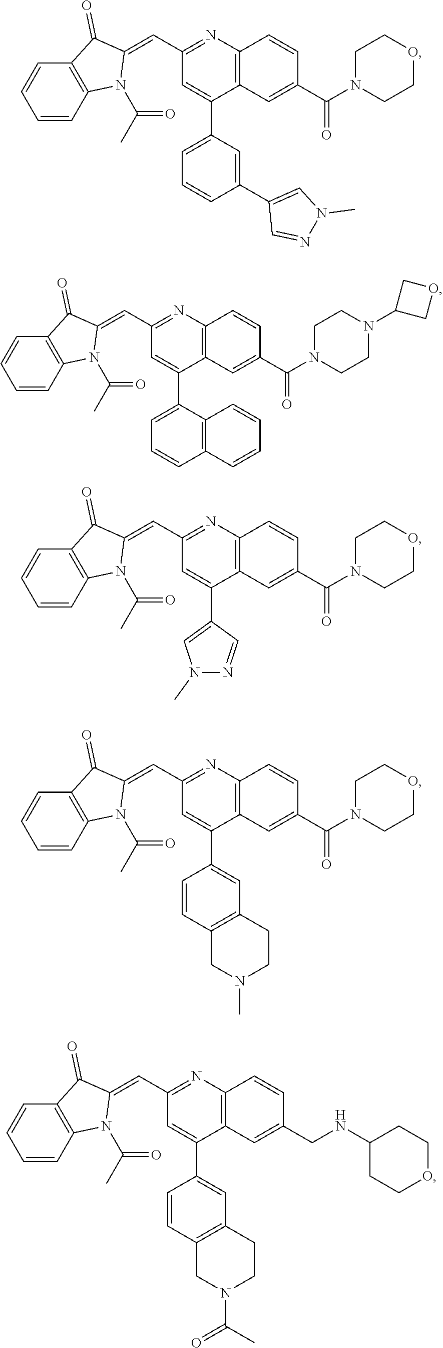 [SMILES: CC(=O)N1/C(=C\c2cc(-c3cccc(-c4cnn(C)c4)c3)c3cc(C(=O)N4CCOCC4)ccc3n2)C(=O)c2ccccc21 ; CC(=O)N1/C(=C\c2cc(-c3cccc4ccccc34)c3cc(C(=O)N4CCN(C5COC5)CC4)ccc3n2)C(=O)c2ccccc21 ; CC(=O)N1/C(=C\c2cc(-c3cnn(C)c3)c3cc(C(=O)N4CCOCC4)ccc3n2)C(=O)c2ccccc21 ; CC(=O)N1/C(=C\c2cc(-c3ccc4c(c3)CCN(C)C4)c3cc(C(=O)N4CCOCC4)ccc3n2)C(=O)c2ccccc21 ; CC(=O)N1CCc2cc(-c3cc(/C=C4/C(=O)c5ccccc5N4C(C)=O)nc4ccc(CNC5CCOCC5)cc34)ccc2C1]

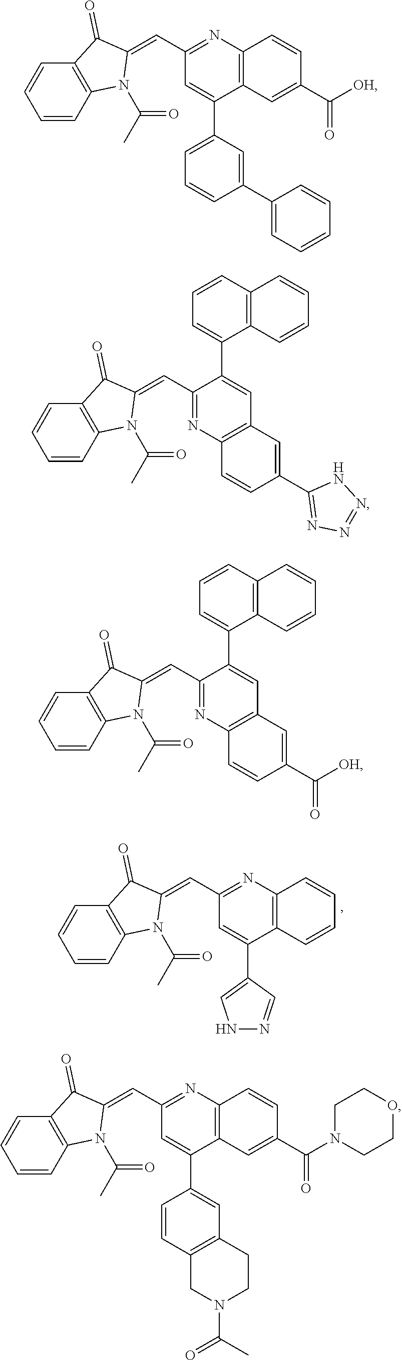

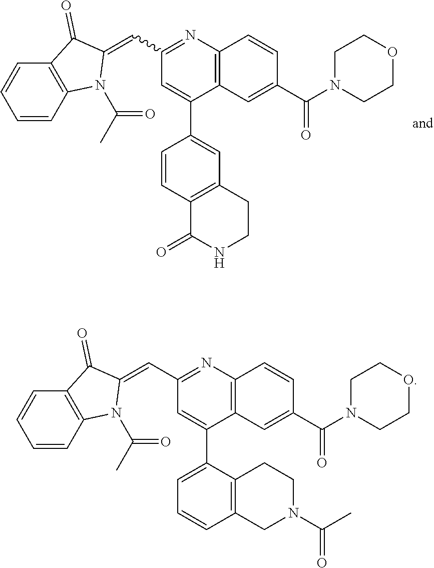 [SMILES: CC(=O)N1C(=Cc2cc(-c3ccc4c(c3)CCNC4=O)c3cc(C(=O)N4CCOCC4)ccc3n2)C(=O)c2ccccc21 ; CC(=O)N1CCc2c(cccc2-c2cc(/C=C3/C(=O)c4ccccc4N3C(C)=O)nc3ccc(C(=O)N4CCOCC4)cc23)C1]

(49) As another aspect of the present invention, it is a method for treating patients having diseases or conditions associated with activation of a Ras/Raf/MEK/ERK signaling pathway (Ras/MAPK pathway), in particular, blood cancers such as leukemia (ALL, APL, and AML) and/or myeloma; and solid cancers such as small cell lung cancer, gastrointestinal cancer, colon cancer, rectal cancer, colorectal cancer, color development, pancreatic cancer, melanoma and/or ovarian cancer, wherein the method comprises administering a Ras/Raf binding inhibitor, comprising a therapeutically effective amount of at least one of, the compound represented by the formula (I) according to the above (1) or a pharmaceutically acceptable salt thereof, or an isomer thereof, or the compound represented by the formula (I) according to the above (26) or a pharmaceutically acceptable salt thereof, or an isomer thereof, to the patients.

(50) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (2) or a pharmaceutically acceptable salt thereof, or an isomer thereof, or the compound represented by the formula (I) according to the above (27) or a pharmaceutically acceptable salt thereof, or an isomer thereof, is used.

(51) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (3) or a pharmaceutically acceptable salt thereof, or an isomer thereof, or the compound represented by the formula (I) according to the above (28) or a pharmaceutically acceptable salt thereof, or an isomer thereof, is used.

(52) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (4) or a pharmaceutically acceptable salt thereof, or an isomer thereof, or the compound represented by the formula (I) according to the above (29) or a pharmaceutically acceptable salt thereof, or an isomer thereof, is used.

(53) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (5) or a pharmaceutically acceptable salt thereof, or an isomer thereof, or the compound represented by the formula (I) according to the above (30) or a pharmaceutically acceptable salt thereof, or an isomer thereof, is used.

(54) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (6) or a pharmaceutically acceptable salt thereof, or an isomer thereof, or the compound represented by the formula (I) according to the above (31) or a pharmaceutically acceptable salt thereof, or an isomer thereof, is used.

(55) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (7) or a pharmaceutically acceptable salt thereof, or an isomer thereof, or the compound represented by the formula (I) according to the above (32) or a pharmaceutically acceptable salt thereof, or an isomer thereof, is used.

(56) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (8) or a pharmaceutically acceptable salt thereof, or an isomer thereof, or the compound represented by the formula (I) according to the above (33) or a pharmaceutically acceptable salt thereof, or an isomer thereof, is used.

(57) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (9) or a pharmaceutically acceptable salt thereof, or an isomer thereof, or the compound represented by the formula (I) according to the above (34) or a pharmaceutically acceptable salt thereof, or an isomer thereof, is used.

(58) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (10) or a pharmaceutically acceptable salt thereof, or an isomer thereof, or the compound represented by the formula (I) according to the above (35) or a pharmaceutically acceptable salt thereof, or an isomer thereof, is used.

(59) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (11) or a pharmaceutically acceptable salt thereof, or an isomer thereof, or the compound represented by the formula (I) according to the above (36) or a pharmaceutically acceptable salt thereof, or an isomer thereof, is used.

(60) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (12) or a pharmaceutically acceptable salt thereof, or an isomer thereof, or the compound represented by the formula (I) according to the above (37) or a pharmaceutically acceptable salt thereof, or an isomer thereof, is used.

(61) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (13) or a pharmaceutically acceptable salt thereof, or an isomer thereof, or the compound represented by the formula (I) according to the above (38) or a pharmaceutically acceptable salt thereof, or an isomer thereof, is used.

(62) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (14) or a pharmaceutically acceptable salt thereof, or an isomer thereof, or the compound represented by the formula (I) according to the above (39) or a pharmaceutically acceptable salt thereof, or an isomer thereof, is used.

(63) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (15) or a pharmaceutically acceptable salt thereof, or an isomer thereof, or the compound represented by the formula (I) according to the above (40) or a pharmaceutically acceptable salt thereof, or an isomer thereof, is used.

(64) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (16) or a pharmaceutically acceptable salt thereof, or an isomer thereof, or the compound represented by the formula (I) according to the above (41) or a pharmaceutically acceptable salt thereof, or an isomer thereof, is used.

(65) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (17) or a pharmaceutically acceptable salt thereof, or an isomer thereof, or the compound represented by the formula (I) according to the above (42) or a pharmaceutically acceptable salt thereof, or an isomer thereof, is used.

(66) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (18) or a pharmaceutically acceptable salt thereof, or an isomer thereof, or the compound represented by the formula (I) according to the above (43) or a pharmaceutically acceptable salt thereof, or an isomer thereof, is used.

(67) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (19) or a pharmaceutically acceptable salt thereof, or an isomer thereof, or the compound represented by the formula (I) according to the above (44) or a pharmaceutically acceptable salt thereof, or an isomer thereof, is used.

(68) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (20) or a pharmaceutically acceptable salt thereof, or an isomer thereof, or the compound represented by the formula (I) according to the above (45) or a pharmaceutically acceptable salt thereof, or an isomer thereof, is used.

(69) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (21) or a pharmaceutically acceptable salt thereof, or an isomer thereof, or the compound represented by the formula (I) according to the above (46) or a pharmaceutically acceptable salt thereof, or an isomer thereof, is used.

(70) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (22) or a pharmaceutically acceptable salt thereof, or an isomer thereof, or the compound represented by the formula (I) according to the above (47) or a pharmaceutically acceptable salt thereof, or an isomer thereof, is used.

(71) As another more preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which a Ras/Raf binding inhibitor, comprising the compound represented by the formula (I) according to the above (23) or a pharmaceutically acceptable salt thereof, or an isomer thereof, or the compound represented by the formula (I) according to the above (48) or a pharmaceutically acceptable salt thereof, or an isomer thereof, is used.

(72) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which the compound represented by the formula (I) according to the above (24) or a pharmaceutically acceptable salt thereof, or an isomer thereof, is used.

(73) As another preferred aspect of the present invention, it is a therapeutic method for leukemia (ALL, APL, and AML), small cell lung cancer, colorectal cancer, pancreatic cancer, and melanoma bearing activation of the Ras/Raf/MEK/ERK signaling pathway (Ras/MAPK pathway), in which the compound represented by the formula (I) according to the above (25) or a pharmaceutically acceptable salt thereof, or an isomer thereof, is used.

According to the present invention, a medicament comprising the above-described compound represented by the formula (I) or a pharmaceutically acceptable salt thereof, or an isomer thereof, is further provided.

According to the present invention, an anticancer agent comprising the above-described compound represented by the formula (I) or a pharmaceutically acceptable salt thereof, or an isomer thereof, is further provided.

### Definitions

The “C₆₋₁₀ aryl group” in the definitions of B, the “C₆₋₁₀ aryl” of the “C₆₋₁₀ arylamino” in the definitions of R¹¹, the “C₆₋₁₀ aryl group” of the “C₆₋₁₀ aryl group optionally having a substituent” in the definitions of R², R³ and R⁴,
the “C₆₋₁₀ aryl group” in the definitions of R⁶ and R⁷, and
the “C₆₋₁₀ aryl” in the “substituent” of the “C₁₋₆ alkyl group optionally having a substituent,” and the “C₆₋₁₀ aryl” of the “C₆₋₁₀ aryloxy,” in the definitions of R¹⁵, mean a monocyclic or bicyclic 6- to 10-membered aromatic ring group. Examples thereof may include phenyl, indenyl and naphthyl, and the C₆₋₁₀ aryl group is preferably a phenyl group.

Besides, the above-described “aryl group” may be annelated with a cycloalkyl group containing 3 to 10 carbon atoms, and for example, it may be a group such as 2-indanyl.

The above-described aryl group is preferably phenyl or naphthyl.

The “substituent” of the “C₆₋₁₀ aryl group optionally having a substituent” in the definitions of R², R³ and R⁴ is the above-described “C₆₋₁₀ aryl” oxy, “a heteroaryl group containing 1 to 4 atoms selected from N, S and O, and optionally having a substituent,” or “phenyl optionally annelated with R¹²,” and it is preferably “a heteroaryl group containing 1 to 4 atoms selected from N, S and O, and optionally having a substituent” (more preferably, pyridyl, phenylpyridyl, quinolyl, indazolyl, pyrazolyl, or methylpyrazolyl; even more preferably, indazolyl, pyrazolyl, or methylpyrazolyl), or “phenyl optionally annelated with an R¹² group” (further preferably, phenyl).

The “heteroaryl group containing 1 to 4 atoms selected from N, S and O” in the definitions of B, the “heteroaryl group containing 1 to 4 atoms selected from N, S and O” in the definitions of R¹⁵, and
the “heteroaryl group containing 1 to 4 atoms selected from N, S and O” of the “heteroaryl group containing 1 to 4 atoms selected from N, S and O, and optionally having a substituent” in the definitions of R², R³ and R⁴ mean an aromatic ring system containing carbon and at least one heteroatom.

The heteroaryl group may be either a monocyclic or polycyclic heteroaryl group.

The heteroaryl group may have 1 to 4 heteroatoms in the ring thereof.

The polycyclic heteroaryl ring may contain a fused, spiro, or crosslinked ring conjugation, and the polycyclic heteroaryl ring is, for example, bicyclic heteroaryl or polycyclic heteroaryl.

The bicyclic heteroaryl ring may contain 8 to 12 ring member atoms.

The monocyclic heteroaryl group may contain 5 to 8 ring member atoms (carbon atoms and heteroatoms).

Examples of such a heteroaryl group may include pyridyl, phenylpyridyl, quinolyl, isoquinolyl, indazolyl, pyrazolyl, pyrazolyl, indolyl, thiazolyl, pyrrolopyridinyl, benzothiazolyl, furopyridinyl, thienyl, furanyl, imidazolyl, isoxazolyl, oxazolyl, pyrrolyl, thiadiazolyl, triazolyl, pyridazinyl, azaindolyl, benzimidazolyl, benzofuranyl, benzothienyl, benzodisoxazolyl, benzoxazolyl, benzopyrazolyl, benzothiadiazolyl, benzotriadiazolyl, benzotriazolyl, and adenyl, but examples of the heteroaryl group are not limited thereto.

The heteroaryl group is preferably quinolyl, indazolyl, pyrazolyl, indolyl, pyridyl, pyrrolopyridinyl, benzothiazolyl, furopyridinyl, or thiazolyl, and is more preferably quinolyl, pyridyl, benzothiazolyl, or thiazolyl, or one of the following groups.

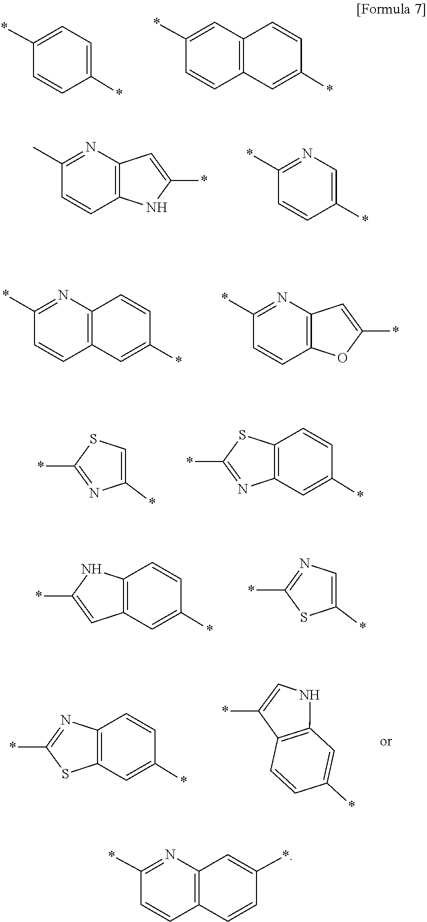

However, one of the above-described bonding hands corresponds to the bonding of the wavy line in the formula (I), and the other bonding hand represents a bonding hand with any one substituent other than the hydrogen in R², R³ and R⁴.

Besides, in such a case, if there is another substituent, the substituent is substituted with the residual position described above.

The “substituent” of the “heteroaryl group containing 1 to 4 atoms selected from N, S and O, and optionally having a substituent” in the definitions of R², R³ and R⁴ is the below-mentioned “C₁₋₆ alkyl group,” is preferably a “C₁₋₄ alkyl group,” and is more preferably methyl.

The “C₁₋₆ alkyl group” in the definitions of R¹,
the “C₁₋₆ alkyl group” of the “C₁₋₆ alkyl group optionally having a substituent” in the definitions of R², R³ and R⁴,
the “C₁₋₆ alkyl” in the definitions of R⁵,
the “C₁₋₆ alkyl group” in the definitions of R⁶ and R⁷,
the “C₁₋₆ alkyl” of the “R¹²—C₁₋₆ alkyl-CONH—” in the definitions of R¹¹,
the “C₁₋₆ alkyl” of the “C₁₋₆ alkyl-CO—” in the definitions of R¹³,
the “C₁₋₆ alkyl group” in the definitions of R¹⁴,
the “C₁₋₆ alkyl group” of the “C₁₋₆ alkyl group optionally having a substituent” in the definitions of R¹⁵, and the “C₁₋₆ alkyl” of “C₁₋₆ alkyl-CO—” and “R¹²—C₁₋₆ alkyl-CO—” in the definitions of R¹⁶ and R¹⁷, mean a saturated monovalent hydrocarbon group having 1, 2, 3, 4, 5 or 6 carbon atoms as a linear or branched sequence, and examples of the alkyl group may include methyl, ethyl, propyl, isopropyl, cyclopropyl, n-butyl, isobutyl, sec-butyl, t-butyl, cyclobutyl, n-pentyl, 3-(2-methyl)butyl, 2-pentyl, 2-methylbutyl, neopentyl, cyclopentyl, n-hexyl, 2-hexyl, 2-methylpentyl, and cyclohexyl. The alkyl group is preferably a linear or branched alkyl group containing 1 to 4 carbon atoms, and is more preferably methyl or ethyl.

The “halogen” in the definitions of R¹, R², R³ and R⁴ is fluoro, chloro, bromo, or iodide, and is preferably F or Cl.

The “heterocyclyl group containing 1 or 2 atoms (groups) selected from N, S, SO, SO₂ and O” of the “heterocyclyl group containing 1 or 2 atoms (groups) selected from N, S, SO, SO₂ and O, and optionally having a substituent” in the definitions of R¹², means a monocyclic or polycyclic non-aromatic ring system containing 1 or 2 atoms (groups) selected from N, S and O, in order to form a ring.

The heterocyclic group preferably comprises, as heteroatoms, an N-oxide, a sulfur oxide, and dioxide, and the ring is preferably a 3- to 10-membered ring, and is completely saturated or is expressed as one or more unsaturation degrees.

Multiple substitution degrees, preferably 1, 2 or 3, are included in the present definition.

Examples of such a heterocyclic group may include morpholino, piperazinyl, thiomorpholino, dioxidothiomorpholino, tetrahydropyranyl, tetrahydrothiopyranyl, pyrrolidinyl, dioxidotetrahydrothiopyranyl, piperidinyl, azetidinyl, oxopiperazinyl, oxopiperidinyl, oxoazepinyl, azepinyl, tetrahydrofuranyl, dioxoranyl, tetrahydroimidazolyl, tetrahydrothiazolyl, tetrahydrooxazolyl morpholinyl, thiomorpholinyl, thiamorpholinyl sulfoxide, thiamorpholinyl sulfone and oxadiazolyl, but the examples of the heterocyclic group are not limited thereto.

The heterocyclic group is preferably morpholino, piperazinyl, piperidinyl, or tetrahydropyranyl.

The “substituent” of the “heterocyclyl containing 1 or 2 atoms (groups) selected from N, S, SO, SO₂ and O, and optionally having a substituent” in the definitions of R¹², is —CO—, —COOH, cyano, 1 or 2 C₁₋₆ alkyl groups (preferably, methyl groups on both sides of the heteroatom selected from N, S, SO, SO₂ and O of the heterocyclyl group, more preferably, 2,6-dimethyl for tetrahydropyranyl and morpholino), C₁₋₆ alkyl-CO— (preferably, acetyl), C₁₋₆ alkyl-SO₂— (e.g. mesyl, etc.), C₆₋₁₀ aryl (preferably, phenyl), 3-methoxy-2-hydroxypropyl, R¹² (preferably, oxetanyl, or morpholino), R¹²CH₂-(preferably, methoxyoxetanylmethyl), R¹²CH₂CO— (preferably, morpholinomethylcarbonyl), R¹²CH₂OCO— (preferably, methoxyoxetanylmethoxycarbonyl), or methoxyethyl.

The “C₁₋₆ alkoxy” that is the “substituent” of the “C₁₋₆ alkyl group optionally having a substituent” in the definition of R¹⁵ means the above-described “C₁₋₆ alkyl group,” which binds to an oxygen atom, and, the C₁₋₆ alkoxy group is, for example, a linear or branched alkoxy group containing 1 to 6 carbon atoms, such as methoxy, ethoxy, n-propoxy, isopropoxy, n-butoxy, isobutoxy, s-butoxy, tert-butoxy, n-pentoxy, isopentoxy, 2-methylbutoxy, neopentoxy, n-hexyloxy, 4-methylpentoxy, 3-methylpentoxy, 2-methylpentoxy, 3,3-dimethylbutoxy, 2,2-dimethylbutoxy, 1,1-dimethylbutoxy, 1,2-dimethylbutoxy, 1,3-dimethylbutoxy, or 2,3-dimethylbutoxy. The C₁₋₆ alkoxy group is preferably a linear or branched alkoxy group containing 1 to 4 carbon atoms.

The “C₆₋₁₀ aryloxy” that is the “substituent” of the “C₁₋₆ alkyl group optionally having a substituent” in the definition of R¹⁵ means the above-described “C₆₋₁₀ aryl group,” which binds to an oxygen atom.

The group represented by the following formula (II) (wherein the wavy line represents a bonding hand) in the formula (I):

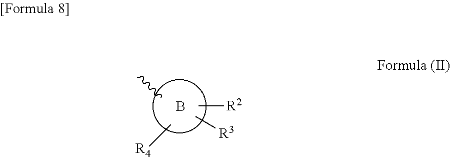

is particularly preferably a group selected from the following:

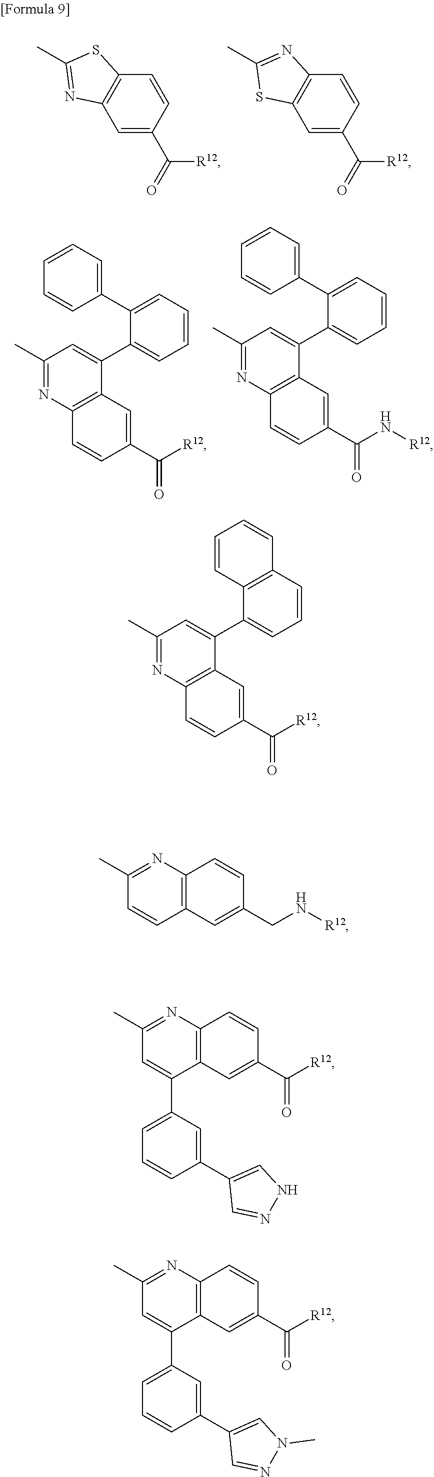

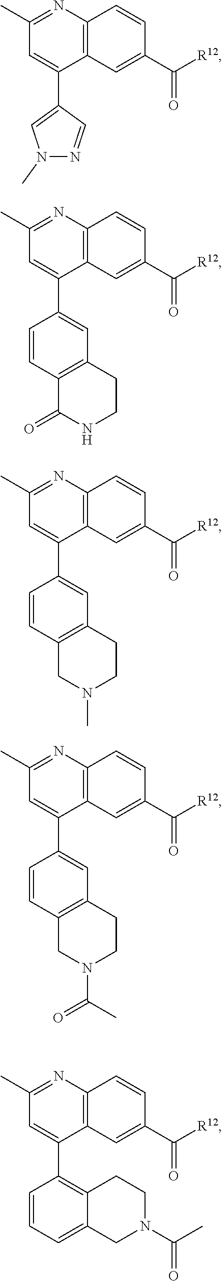

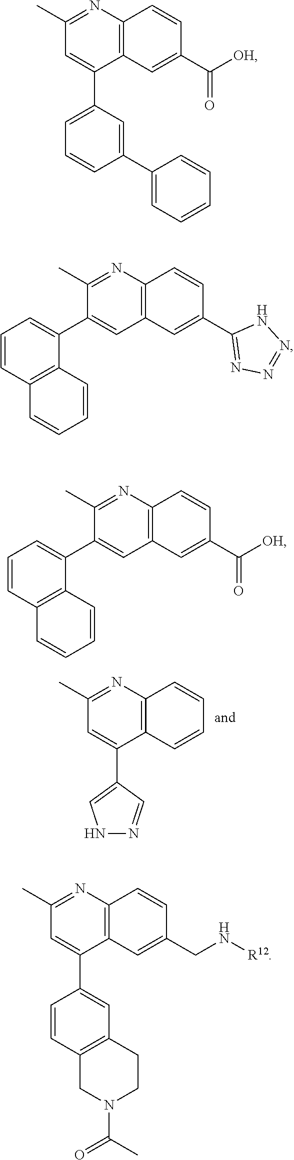

The wavy line represents an E-form or Z-form geometric isomer.

As described above, geometric isomers are present in the compound of the present invention, and thus, the present invention includes compounds from which these isomers are separated, or mixtures comprising these isomers at any proportions.

Moreover, the compound of the present invention may have an asymmetric carbon atom in some cases. Based on such asymmetric carbon atoms, diastereomers and (R)- and (S)-form optical isomers may exist.

The present invention encompasses all of such mixtures of optical isomers and all isolates thereof, possible diastereomers, as well as their racemic mixtures, their substantially pure split enantiomers, all possible geometric isomers, and pharmaceutically acceptable salts thereof.

During the course of the synthetic procedures used to prepare such compounds, or upon using racemization or epimerization procedures known to those skilled in the art, the products generated from such procedures may become a mixture of stereoisomers.

Furthermore, in a case where tautomers of the compounds of the formula (I) exist, the present invention includes any possible tautomers and pharmaceutically acceptable salts thereof, and mixtures thereof, unless otherwise specified.

The salt of the compound of the present invention, which is for use in a medicament, means a non-toxic “pharmaceutically acceptable salt.”

The form of the pharmaceutically acceptable salt includes a pharmaceutically acceptable acidic/anionic or basic/cationic salt.

When the compound (I) of the present invention is an acidic compound, its corresponding salt can be conveniently prepared from pharmaceutically acceptable non-toxic bases, including inorganic and organic bases.

When the compound (I) of the present invention is a basic compound, its corresponding salt can be conveniently prepared from pharmaceutically acceptable non-toxic acids, including inorganic acids and organic acids.

Such a salt is a pharmaceutically acceptable salt, and preferred examples of such a pharmaceutically acceptable salt may include: acid-added salts with inorganic acids such as hydrochloric acid, hydrobromic acid, hydroiodic acid, perchloric acid, sulfuric acid, nitric acid or phosphoric acid, and with organic acids such as formic acid, acetic acid, propionic acid, oxalic acid, malonic acid, succinic acid, fumaric acid, maleic acid, lactic acid, malic acid, tartaric acid, citric acid, methanesulfonic acid, benzenesulfonic acid, aspartic acid, glutamic acid, glycolic acid, benzoic acid, mandelic acid, hydroxyethanesulfonic acid, pamoic acid, 2-naphthalenesulfonic acid, p-toluenesulfonic acid, cyclohexanesulfamic acid, salicylic acid, saccharinic acid or trifluoroacetic acid; salts with inorganic bases such as sodium, lithium, potassium, magnesium, calcium, aluminum or zinc, and with organic bases such as methylamine, ethylamine, ethanolamine, lysine, ornithine, chloroprocaine, choline, diethanolamine or ethylenediamine; and ammonium salts, but the examples of the pharmaceutically acceptable salt are not limited thereto.

When the compound of the formula (I) and a pharmaceutically acceptable salt thereof are present in the form of a solvate or a polymorph, the present invention includes any possible solvate and polymorph forms. The type of a solvent used to form the solvate is not particularly limited, as long as the solvent is pharmaceutically acceptable. For example, water, ethanol, propanol, acetone, etc. can be used as such a solvent.

The present invention includes a prodrug of the compound of the present invention within the scope thereof. In general, such a prodrug is a functional derivative of the compound, which is easily converted to a required compound in vivo.

Accordingly, in the present invention, the term “compound” encompasses specifically disclosed compounds, or compounds that are not specifically disclosed but are converted to specific compounds in vivo after administration thereof to the subject, and are used to treat various disorders described herein.

Conventional procedures for the selection and preparation of appropriate prodrug derivatives are described, for example, in “Design of Prodrugs,” edited by H. Bundgaard, Elsevier, 1985, and the contents thereof are incorporated in the present description.

The term “inhibitor” as used herein encompasses a product containing a designated component in a designated amount, and any given product directly or indirectly generated from a combination of designated components in designated amounts.

Accordingly, a composition containing, as an active ingredient, the compound (I) of the present invention, etc., and a method for preparing the present compound are also parts of the present invention.

The pharmaceutical composition of the present invention comprises the compound represented by the formula (I) or a pharmaceutically acceptable salt thereof (or an isomer thereof) as an active ingredient, a pharmaceutically acceptable carrier, and optionally, other therapeutic ingredients or adjuvants.

The composition includes those suitable for oral, rectal, topical, and parenteral (including subcutaneous, intramuscular, and intravenous) administrations, although the most appropriate administration route in any given case depends on a specific host and the nature and severity of a condition for which the active ingredient is administered.

The composition may be conveniently provided in a unit dosage form and may be prepared by any of the methods publicly known in the field of pharmacy.

In practice, the compound represented by the formula (I) of the invention, or a prodrug or a metabolite or a pharmaceutically acceptable salt thereof, which serves as an active ingredient, is closely mixed with a pharmaceutical carrier according to conventional pharmaceutical formulation techniques, and thereafter, a thus generated product can be conveniently molded into a desired form.

The carrier can take a variety of forms, depending on the form of a preparation desired for administration, for example, an oral or parenteral (including intravenous) form.

Therefore, the composition of the present invention can be provided as a separate unit suitable for oral administration, such as capsules, cachets or tablets, each containing a predetermined amount of active ingredient.

Furthermore, the composition can be provided as powders, as granules, as a solution, as a suspension in an aqueous liquid, as a non-aqueous liquid, as an oil-in-water emulsion, or as a water-in-oil liquid emulsion.

In addition to the above-described common dosage forms, the compound represented by the formula (I) or a pharmaceutically acceptable salt thereof, etc. can also be administered by a controlled release means and/or delivery device.

Moreover, the compound of the formula (I) or a pharmaceutically acceptable salt thereof can be combined with one or more other therapeutically active compounds, and can be comprised in a pharmaceutical composition.

The pharmaceutical carrier used can be, for example, a solid or liquid carrier.

Examples of such a solid carrier may include lactose, terra alba, sucrose, talc, gelatin, agar, pectin, acacia, magnesium stearate, and stearic acid.

Examples of such a liquid carrier may include sugar syrup, peanut oil, olive oil, and water.

The administration form of the compound (I) of the present invention can be, for example, oral administration using tablets, capsules, granules, powder agents or syrup agents, or parenteral administration using injections or suppositories. These formulations are produced according to publicly known methods, using additives including: excipients (e.g., organic excipients including: sugar derivatives such as lactose, white sugar, glucose, mannitol and sorbitol; starch derivatives such as corn starch, potato starch, α-starch and dextrin; cellulose derivatives such as crystalline cellulose; rubber Arabic; dextran; and pullulan; and inorganic excipients including: silicate derivatives such as light anhydrous silicic acid, synthetic aluminum silicate, calcium silicate and magnesium metasilicate aluminate; phosphates such as calcium hydrogen phosphate; carbonates such as calcium carbonate; and sulfates such as calcium sulfate, can be listed), lubricants (e.g., metallic stearates such as stearic acid, calcium stearate and magnesium stearate; talc; colloidal silica; waxes such as v veegum and spermaceti; boric acid; adipic acid; sulfates such as sodium sulfate; glycol; fumaric acid; sodium benzoate; DL-leucine; sodium salts of fatty acids; lauryl sulfates such as sodium lauryl sulfate and magnesium lauryl sulfate; silicates such as silicic anhydride and silicic acid hydrate; and the above starch derivatives, can be listed), binders (e.g. hydroxypropyl cellulose, hydroxypropyl methyl cellulose, polyvinyl pyrrolidone, macrogol, and the same compounds as those for the above-described excipients, can be listed), disintegrators (e.g., cellulose derivatives such as low-substituted hydroxypropyl cellulose, carboxymethyl cellulose, calcium carboxymethyl cellulose, and internally cross-linked sodium carboxymethyl cellulose; and chemically modified starches such as carboxymethyl starch, sodium carboxymethyl starch, and cross-linked polyvinyl pyrrolidone, can be listed), stabilizers (paraxybenzoic acid esters such as methylparaben and propylparaben; alcohols such as chlorobutanol, benzyl alcohol, and phenylethyl alcohol; benzalkonium chloride; phenols such as phenol and cresol; thimerosal; dehydroacetic acid; and sorbic acid, can be listed), flavoring agents (e.g., usually used sweeteners, acidifiers, flavors, etc., can be listed), diluents, and the like.

A tablet containing the composition of the present invention can be prepared by compressing or molding the present composition, together with optionally selected one or more auxiliary ingredients or adjuvants. A compressed tablet can be prepared by mixing the active ingredient in a free floating form, such as powders or granules, optionally with a binder, a lubricant, an inactive diluent, a surfactant or a dispersant, and then compressing the mixture, using a suitable machine. A molded tablet can be produced by molding a mixture of powdered compounds moistened with an inactive liquid diluent in a suitable machine.

The pharmaceutical composition of the present invention suitable for injectable use includes a sterile aqueous solution or dispersion. In addition, the composition may be in the form of sterile powders for immediate preparation of such a sterile injectable solution or dispersion. In all of the cases, the final injectable form must be sterile, and must be a fluid effective for easy injectability. The pharmaceutical composition must be stable under the conditions of production and storage; therefore, it should preferably be preserved against contamination by microorganisms such as bacteria and fungi. The carrier can be a solvent or a dispersing medium containing, for example, water, ethanol, polyols (e.g. glycerol, propylene glycol, and liquid polyethylene glycol), vegetable oil, and a suitable mixture thereof.

Furthermore, the composition can be in a form suitable for use in transdermal devices. These preparations can be prepared by conventional processing methods, using the compound represented by the formula (I) of the present invention or a pharmaceutically acceptable salt thereof.

The applied dose of the composition is different depending on symptoms, age, administration method, etc. For example, in the case of oral administration, the lower limit is 0.01 mg/kg body weight (preferably, 0.1 mg/kg body weight) per dose, and the upper limit is 300 mg/kg body weight (preferably, 200 mg/kg body weight) per dose. In the case of intravenous administration, the lower limit is 0.001 mg/kg body weight (preferably, 0.01 mg/kg body weight) per dose, and the upper limit is 100 mg/kg body weight (preferably, 30 mg/kg body weight) per dose. Such a dose is desirably administered once or several times per day, depending on symptoms.

However, it is understood that the specific dose level for any given specific patient will depend on various factors including age, body weight, general health, gender, diet, time of administration, route of administration, elimination rate, drug combination, and severity of a specific disease being treated with the composition.

In several embodiments, the above-described compound is in a weight ratio of about 0.0001 to about 10 to the excipient.

In several embodiments, the above-described compound is in a weight ratio of about 0.0005 to about 0.25 to the excipient.

### Advantageous Effects of Invention

Since the compound of the present invention and a pharmaceutical composition comprising the same exhibit a Ras/Raf signaling inhibitory action on cancer cells having drug resistance, and even on a wide variety of Ras mutant cancers, the present compound and the present pharmaceutical composition specifically inhibit Ras/Raf binding and exhibit novel excellent effects on many cancers including, as typical examples, leukemia (ALL, APL, and AML), small cell lung cancer, colorectal cancer, pancreatic cancer, and melanoma.

### INDUSTRIAL APPLICABILITY

The present invention provides: a novel excellent low-molecular-weight compound that specifically inhibits Ras/Raf binding; a pharmaceutical composition comprising the same; a method for producing the above-described pharmaceutical composition, using such a compound; and also, a novel excellent therapeutic method for many cancers including, as typical examples, leukemia (ALL, APL, and AML), small cell lung cancer, colorectal cancer, pancreatic cancer, and melanoma bearing activation of the Ras/Raf/MEK/ERK signaling pathway (Ras/MAPK pathway).

### Embodiment of Carrying Out the Invention

### (Production Method)

The compound of the present invention and a pharmaceutically acceptable salt thereof, and an isomer thereof, can be produced by applying various known synthetic methods, which are sufficiently favorable to skilled persons in the technical field of organic synthesis described below, unless otherwise specified.

The compound of the formula (I) can be synthesized by referring to the following methods.

Preferred methods are not limited to the methods described below. Reference documents cited herein are incorporated herein by reference in their entirety

The synthetic methods described below are intended as examples of the present invention, and are not intended to limit the scope of the claimed compounds by their subject matters and these examples.

When the preparation of starting compounds is not described, those starting compounds are commercially available or may be prepared in the same manner as that for the known compounds or methods described herein.

The substances described in the references are prepared according to the published synthetic methods.

As described in the present description, the final compound is a generated product having the structural formula shown as formula (I).

It is understood that any given compound of the formula (I) can be prepared by the selection of reagents having appropriate substitutions. Solvents, temperatures, pressures, and other reaction conditions can be easily selected by those skilled in the art.

At that time, in each step, functional groups are protected and deprotected as necessary, so that a desired compound can be obtained. The protection and deprotection of functional groups can be performed by known methods, for example, by the method described in “Green's Protective Groups in Organic Synthesis” 5th edition written by Wuts.

Hereinafter, the present invention will be more specifically described in the following examples, production examples, and test examples.

### EXAMPLES

The present invention will be more readily understood by referring to the following examples. Besides, it should be understood that although the present invention is further defined with the following examples, these examples are used merely to illustrate specific aspects and embodiments of the present invention.

Those skilled in the art can determine the essential features of the present invention with certainty, and can make various modifications to adapt the present invention to various intended uses and conditions, without departing from the spirit and scope of the present invention.

Therefore, the present invention is not limited by the illustrative examples described below in the present description, but rather, is specified by the claims appended hereto.

It is to be noted that the methods for producing the raw material compounds used in the following Examples will be described in the following Production Examples.

In the following examples, the following abbreviations may be used in some cases.
mCPBA: methachloroperbenzoic acid
THF: tetrahydrofuran
DMF: N,N-dimethylformamide
DMA: N,N-dimethylacetamide
DIPEA: N,N-diisopropylethylamine
HATU: 1-[bis(dimethylamino)methylene]-1H-1,2,3-triazolo[4,5-b]pyridinium 3-oxide hexafluorophosphate
TFA: 2,2,2-trifluoroacetic acid
WSCD HCl: 1-ethyl-3-(3-dimethylaminopropyl)carbodiimide hydrochloride
DCE: 1,2-dichloroethane
BINAP: (1,1′-binaphthalene-2,2′-diyl)bis(diphenylphosphane)
X-Phos: 2-dicyclohexylphosphino-2′,4′,6′-triisopropyl-1,1′-biphenyl
1H NMR: proton nuclear magnetic resonance
MS: mass spectrometry using electrospray ionization method
(M+H), (M+Na), and (M+H₂O+H): molecular ion peaks
M: molarity
Tr: trityl group
SEM: (2-(trimethylsilyl)ethoxy)methyl group

### Production Example 1

A mixture of N-((3-formyl-1H-indol-4-yl)methyl)acetamide (manufactured by Aurora Fine Chemicals; 601 mg) and acetic anhydride (5.85 ml) was stirred at 145° C. for 4 hours, and thereafter, under a nitrogen flow, the solvent was removed from the reaction mixture. The obtained residue was crystallized from methanol to obtain N-acetyl-N-((1-acetyl-3-formyl-1H-indol-4-yl)methyl)acetamide (518 mg) in the form of a light brown solid.

A mixture of N-acetyl-N-((1-acetyl-3-formyl-1H-indol-4-yl)methyl)acetamide (485 mg), dichloromethane (7 ml), and 70% mCPBA (478 mg) was stirred at room temperature for 22 hours 50 minutes, and thereafter, 70% mCPBA (119 mg) was added to the reaction mixture. The thus obtained mixture was further stirred at room temperature for 2 hours 30 minutes.

Subsequently, chloroform was added to the reaction mixture, and the thus obtained mixture was washed with a saturated sodium hydrogen carbonate aqueous solution, and the organic layer was then dried over anhydrous sodium sulfate. The solvent was distilled away under reduced pressure, and methanol (6 ml) and potassium carbonate (12 mg) were then added to the obtained residue. Thereafter, the obtained mixture was stirred at room temperature for 2 minutes.

The solvent was distilled away from the reaction mixture under reduced pressure, and the obtained residue was then purified by silica gel column chromatography (eluent: hexane-chloroform-methanol), so as to obtain N-acetyl-N-((1-acetyl-3-oxoindolin-4-yl)methyl)acetamide (86 mg) in the form of a light brown solid.

### Production Example 2

A mixture of N-acetyl-N-((1-acetyl-3-oxoindolin-4-yl)methyl)acetamide (Production Example 1; 86 mg), ethanol (4 ml), THF (2 ml), water (2 ml), and sodium hydrogen carbonate (30 mg) was stirred at room temperature for 28 hours.

Thereafter, sodium hydrogen carbonate (16 mg) was added to the reaction mixture, and the thus obtained mixture was further stirred at room temperature for 2 days. After that, the solvent was distilled away from the reaction mixture under reduced pressure, and toluene was then added to the residue, and thereafter, the solvent was distilled away under reduced pressure.

The obtained residue was purified by silica gel column chromatography (eluent: chloroform-methanol), so as to obtain N-((1-acetyl-3-oxoindolin-4-yl)methyl)acetamide (15 mg) in the form of a light brownish-yellow solid.

### Production Example 3

A mixture of 4-bromo-3-methoxybenzaldehyde (manufactured by AstaTech; 215 mg), acetonitrile (5 ml), triethylamine (5 ml), acrylamide (78 mg), and tris(2-methylphenyl)phosphine (61 mg) was degassed by repeating pressure reduction and nitrogen substitution, and palladium acetate (22 mg) was then added to the reaction mixture. The thus obtained mixture was stirred under heat reflux for 3 hours 30 minutes. Thereafter, 2 M hydrochloric acid was added to the reaction mixture, and a generated product was then extracted with ethyl acetate.

The extract was dried over anhydrous sodium sulfate, and the solvent was then distilled away under reduced pressure. The obtained residue was purified by silica gel column chromatography (eluent: chloroform-methanol), so as to obtain (E)-3-(4-formyl-2-methoxyphenyl)acrylamide (97 mg) in the form of a light yellow solid.

### Production Example 4

A mixture of 6-bromo-2-methylbenzo[d]thiazole (manufactured by Combi-Blocks; 200 mg), DMA (4 ml), triethylamine (0.367 ml), 4-(vinylsulfonyl)morpholine (Organic Letters, 22(13), 4970-4973, 2020; 311 mg), and tris(2-methoxyphenyl)phosphine (53 mg) was degassed by repeating pressure reduction and nitrogen substitution, and palladium acetate (20 mg) was then added to the reaction mixture. The thus obtained mixture was stirred at 100° C. for 20 hours.

Thereafter, triethylamine (0.367 ml), 4-(vinylsulfonyl)morpholine (311 mg), and tris(2-methoxyphenyl)phosphine (133 mg) were added to the reaction mixture, and the thus obtained mixture was then degassed by repeating pressure reduction and nitrogen substitution. After that, palladium acetate (49 mg) was added to the reaction mixture, and the thus obtained mixture was then stirred at 100° C. for 26 hours. Thereafter, the solvent was removed from the reaction mixture under a nitrogen flow, and the obtained residue was then purified by silica gel column chromatography (eluent: hexane-chloroform-ethyl acetate), so as to obtain (E)-4-((2-(2-methylbenzo[d]thiazol-6-yl)vinyl)sulfonyl)morpholine (32 mg) in the form of a brown solid.

### Production Example 5

2-Chloroethanesulfonyl chloride (1.62 ml) was added dropwise to a mixture of 28% ammonia water (4.3 ml) and chloroform (5 ml) under ice cold, and the obtained mixture was then stirred at room temperature for 1 hour. Thereafter, toluene was added to the reaction mixture, and the solvent was then distilled away under reduced pressure. A mixture of the obtained residue and THF (100 ml) was stirred at 60° C. for 30 minutes, and a generated solid was removed by filtration. The solvent was distilled away from the filtrate under reduced pressure.

To the obtained residue, 4-bromo-3-methoxybenzaldehyde (manufactured by AstaTech; 100 mg), DMA (2 ml), triethylamine (0.194 ml), and tris(2-methoxyphenyl)phosphine (28 mg) were added, and the obtained mixture was then degassed by repeating pressure reduction and nitrogen substitution. After that, palladium acetate (11 mg) was added to the reaction mixture, and the thus obtained mixture was then stirred at 100° C. for 5 hours. Thereafter, the solvent was removed from the reaction mixture under a nitrogen flow, and the obtained residue was then purified by silica gel column chromatography (eluent: chloroform-methanol), so as to obtain (E)-2-(4-formyl-2-methoxyphenyl)ethene-1-sulfonamide (69 mg) in the form of a yellow oily substance.

### Production Example 6

A mixture of (E)-3-(4-formyl-2-methoxyphenyl)acrylamide (Production Example 3; 105 mg), ethanol (2 ml), THF (1 ml), and 5% palladium carbon (14 mg) was stirred under 1 atm of hydrogen at room temperature for 3 hours.

Thereafter, an insoluble matter in the reaction mixture was removed by filtration, and the solvent was then distilled away from the filtrate under reduced pressure. The obtained residue was purified by silica gel column chromatography (eluent: chloroform-methanol), so as to obtain 3-(4-formyl-2-methoxyphenyl)propanamide (76 mg) in the form of a slight yellow solid.

### Production Example 7

A mixture of (E)-3-(2-methylquinolin-6-yl)-1-morpholinoprop-2-en-1-one (Production Example 18; 116 mg), ethanol (2.3 ml), water (0.23 ml), and 20% palladium hydroxide carbon (23 mg) was stirred under 1 atm of hydrogen at room temperature for 22 hours.

Thereafter, an insoluble matter in the reaction mixture was removed by filtration, and the solvent was then distilled away from the filtrate under reduced pressure. After that, toluene was added to the obtained residue, and the solvent was then distilled away again under reduced pressure.

The obtained residue was then purified by silica gel column chromatography (eluent: hexane-chloroform-methanol), so as to obtain 3-(2-methylquinolin-6-yl)-1-morpholinopropan-1-one (107 mg) in the form of a light yellow solid.

### Production Example 8

To a mixture of 3-methoxy-4-((methylthio)methoxy)benzaldehyde (Tetrahedron Letters, 18(6), 533-534, 1977; 139 mg) and dichloromethane (3 ml), 70% mCPBA (226 mg) was added under ice cold, and the obtained mixture was then stirred under ice cold for 40 minutes.

Thereafter, dichloromethane was added to the reaction mixture, and the thus obtained mixture was then washed with was washed with a sodium carbonate aqueous solution. After that, the organic layer was dried over anhydrous sodium sulfate, and the solvent was then distilled away under reduced pressure.

The obtained residue was purified by silica gel column chromatography (eluent: chloroform-methanol), and the previously eluted low-polarity 3-methoxy-4-((methylsulfonyl)methoxy)benzaldehyde (89 mg) was obtained in the form of a colorless solid.

### Production Example 9

In the silica gel column chromatography, in which the compound of Production Example 8 was obtained, the subsequently eluted high-polarity 3-methoxy-4-((methylsulfinyl)methoxy)benzaldehyde (57 mg) was obtained in the form of a colorless solid.

### Production Example 10

A mixture of 4-hydroxy-3-isopropoxybenzaldehyde (WO2011125006; 352 mg), DMF (10 ml), potassium carbonate (810 mg), 2-chloroacetamide (438 mg) was stirred at 80° C. for 17 hours, and the solvent was then distilled away from the reaction mixture under reduced pressure. The obtained residue was purified by silica gel column chromatography (eluent: chloroform-methanol), so as to obtain 2-(4-formyl-2-isopropoxyphenoxy)acetamide (251 mg) in the form of a colorless solid.

### Production Example 14

Using 6-(chloromethyl)-2-methylquinoline hydrochloride (manufactured by BLD Pharmatech), acetonitrile, potassium carbonate, and thiomorpholine 1,1-dioxide, 4-((2-methylquinolin-6-yl)methyl)thiomorpholine 1,1-dioxide was obtained in the form of a colorless solid in the same manner as that of Production Example 10.

### Production Example 16

A mixture of 4-amino-3-methoxybenzaldehyde (manufactured by Sigma-Aldrich; 39 mg), 2-morpholinoacetic acid hydrochloride (manufactured by Tokyo Chemical Industry Co., Ltd.; 70 mg), DMF (0.5 ml), DIPEA (0.157 ml), and HATU (196 mg) was stirred at 60° C. for 20 hours, and the solvent was then removed from the reaction mixture under a nitrogen flow. The obtained residue was successively purified by silica gel column chromatography (eluents: hexane-ethyl acetate, and then, chloromethyl-methanol), so as to obtain N-(4-formyl-2-methoxyphenyl)-2-morpholinoacetamide (35 mg) in the form of a light yellow solid.

### Production Example 18

A mixture of (E)-3-(2-methylquinolin-6-yl)acrylic acid (Organic Letters, 14(21), 5420-5423, 2012; 100 mg), dichloromethane (4 ml), morpholine (0.0615 ml), DIPEA (0.147 ml), and HATU (214 mg) was stirred at room temperature for 16 hours, and the reaction mixture was then purified by silica gel column chromatography (eluent: chloroform-methanol), so as to obtain (E)-3-(2-methylquinolin-6-yl)-1-morpholinoprop-2-en-1-one (121 mg) in the form of a colorless solid.

### Production Example 30

A mixture of 4-([1,1′-biphenyl]-2-yl)-2-methylquinoline-6-carboxylic acid (Production Example 94; 66 mg), 1-(oxetan-3-yl)piperidin-4-amine bis(2,2,2-trifluoroacetate) (manufactured by Enamine; 75 mg), dichloromethane (1.3 ml), DIPEA (0.119 ml), and HATU (89 mg) was stirred at room temperature for 25 hours, and thereafter, ethyl acetate was added to the reaction solution. The obtained solution was successively washed with water, a saturated sodium hydrogen carbonate aqueous solution, and a saline.

The organic layer was dried over anhydrous sodium sulfate, and the solvent was then distilled away under reduced pressure. The obtained residue was successively purified by silica gel column chromatography (eluent: hexane-chloroform-methanol) and then, by gel permeation chromatography (eluent: chloroform), so as to obtain 4-([1,1′-biphenyl]-2-yl)-2-methyl-N-(1-(oxetan-3-yl)piperidin-4-yl)quinoline-6-carboxamide (76 mg) in the form of a colorless oily substance.

### Production Example 31

A mixture of 4-amino-2-methylquinoline-6-carboxylic acid dihydrochloride (Production Example 93; 51 mg), morpholine (0.0274 ml), DMF (2 ml), DIPEA (0.109 ml), and HATU (95 mg) was stirred at room temperature for 16 hours, and the solvent was then removed from the reaction mixture under a nitrogen flow.

The obtained residue was purified by aminopropyl silica gel column chromatography (eluent: chloroform-methanol), so as to obtain (4-amino-2-methylquinolin-6-yl)(morpholino)methanone (63 mg) in the form of a light brown foam product.

### Production Example 33

A mixture of 4-chloro-2-methylquinoline-6-carboxylic acid (Production Example 92; 93 mg), DMF (1.9 ml), morpholine (0.0546 ml), DIPEA (0.130 ml), and HATU (190 mg) was stirred at room temperature for 18 hours, and a solid precipitated in the reaction mixture was then collected by filtration.

The obtained solid was successively washed with DMF, and then with hexane, so as to obtain (4-((3H-[1,2,3]triazolo[4,5-b]pyridin-3-yl)oxy)-2-methylquinolin-6-yl)(morpholino)methanone (144 mg) in the form of a colorless solid.

### Production Example 34

A mixture of 4-chloro-2-methylquinoline-6-carboxylic acid (Production Example 92; 34 mg), dichloromethane (0.68 ml), morpholine (0.0201 ml), and WSCD HCl (35 mg) was stirred at room temperature for 16 hours.

Thereafter, the reaction mixture was purified by silica gel column chromatography (eluent: chloroform-methanol), so as to obtain (4-chloro-2-methylquinolin-6-yl)(morpholino)methanone (40 mg) in the form of a colorless solid.

### Production Example 45

Using (2-methylquinolin-6-yl)methanamine (manufactured by Enamine), 2-morpholinoacetic acid hydrochloride (manufactured by Tokyo Chemical Industry Co., Ltd.), dichloromethane, triethylamine, and WSCD HCl, N-((2-methylquinolin-6-yl)methyl)-2-morpholinoacetamide was obtained in the form of a light yellow oily substance in the same manner as that of Production Example 34.

### Production Example 47

A mixture of methyl 2-(hydroxymethyl)-1-methyl-1H-indole-5-carboxylate (manufactured by Azepine; 61 mg), methanol (1.2 ml), and a 1 M sodium hydroxide aqueous solution (1.2 ml) was stirred at room temperature for 5 hours, and 1,4-dioxane (0.5 ml) was then added to the reaction mixture. The thus obtained mixture was further stirred at room temperature for 19 hours.

Thereafter, 1 M hydrochloric acid (1 ml) was added to the reaction mixture, and the solvent was then distilled away under reduced pressure. After that, toluene was added to the residue, and the solvent was then distilled away again under reduced pressure.

To the obtained residue, dichloromethane (1.2 ml), morpholine (0.0365 ml), and WSCD HCl (80 mg) were added, and the obtained mixture was then stirred at room temperature for 22 hours. Thereafter, the reaction mixture was purified by silica gel column chromatography (eluent: chloroform-methanol), so as to obtain (2-(hydroxymethyl)-1-methyl-1H-indol-5-yl)(morpholino)methanone (60 mg) in the form of a light yellow oily substance.

### Production Example 48

Anhydride (0.132 ml) was added to a mixture of (2-methylquinolin-6-yl)methanamine (manufactured by Enamine; 212 mg), dichloromethane (4 ml), and triethylamine (0.243 ml) under ice cold, and the obtained mixture was then stirred at room temperature for 17 hours. Thereafter, ethyl acetate was added to the reaction mixture, and the thus obtained mixture was successively washed with water and a saturated saline. After that, the organic layer was dried over anhydrous sodium sulfate, and the solvent was then distilled away under reduced pressure.

The obtained residue was purified by silica gel column chromatography (eluent: chloroform-methanol), so as to obtain N-((2-methylquinolin-6-yl)methyl)acetamide (136 mg) in the form of a light yellow solid.

### Production Example 50

A mixture of (4-amino-2-methylquinolin-6-yl)(morpholino)methanone (Production Example 31; 114 mg), dichloromethane (2.3 ml), DMF (0.23 ml), triethylamine (0.0701 ml), and acetic anhydride (0.038 ml) was stirred at room temperature for 21 hours, and thereafter, DMF (0.92 ml) and acetic anhydride (0.112 ml) were added to the reaction mixture. The obtained mixture was stirred at room temperature for 20 hours.

Thereafter, DMF (1.15 ml) and triethylamine (0.140 ml) were added to the reaction mixture, and the thus obtained mixture was further stirred at room temperature for 4 days. After that, the solvent was then removed from the reaction mixture under a nitrogen flow.

The obtained residue was purified by aminopropyl silica gel column chromatography (eluent: chloroform-methanol), so as to obtain N-(2-methyl-6-(morpholine-4-carbonyl)quinolin-4-yl)acetamide (56 mg) in the form of a colorless oily substance.

### Production Example 51

Diisopropyl azodicarboxylate (0.186 ml) was added to a mixture of 6-hydroxy-[1,1′-biphenyl]-3-carbaldehyde (manufactured by Enamine; 125 mg), 2-hydroxy-1-morpholinoethan-1-one (manufactured by AstaTech; 92 mg), THF (2.5 ml), and triphenylphosphine (248 mg), and the thus obtained mixture was then stirred under ice cold at room temperature for 5 hours.

Thereafter, the solvent was distilled away from the reaction mixture under reduced pressure, and then, the obtained residue was successively purified by silica gel column chromatography (eluents: chloroform-ethyl acetate, and then, hexane-chloroform-ethyl acetate), so as to obtain 6-(2-morpholino-2-oxoethoxy)-[1,1′-biphenyl]-3-carbaldehyde (126 mg) in the form of a light yellow oily substance.

### Production Example 54

Methanesulfonic acid anhydride (70 mg) was added to a mixture of 6-fluoro-5-hydroxypicolinaldehyde (manufactured by Milestone Pharma Tech; 47 mg), dichloromethane (1 ml), triethylamine (0.0696 ml), N,N-dimethylpyridin-4-amine (4.1 mg) under ice cold methane, and the obtained mixture was then stirred at room temperature for 3 hours. Thereafter, dichloromethane was added to the reaction mixture, followed by washing with a saturated saline.

The organic layer was dried over anhydrous sodium sulfate, and the solvent was then distilled away under reduced pressure. The obtained residue was purified by silica gel column chromatography (eluent: hexane-ethyl acetate), so as to obtain 2-fluoro-6-formylpyridin-3-yl methanesulfonate (56 mg) in the form of a colorless oily substance.

### Production Example 56

60% Sodium hydride (44 mg) was added to a mixture of methyl 1H-pyrrolo[3,2-b]pyridine-5-carboxylate (Bioorganic & Medicinal Chemistry Letters, 20(1), 413-417, 2010; 147 mg) and DMF (1.47 ml) under ice cold, and the obtained mixture was then stirred at 0° C. for 30 minutes. Thereafter, methanesulfonyl chloride (0.071 ml) was added to the reaction mixture, and the thus obtained mixture was then stirred at room temperature for 21 hours.

Thereafter, water was added to the reaction mixture, and a generated product was then extracted with ethyl acetate. After that, the extract was successively washed with water and a saturated saline. The organic layer was dried over anhydrous sodium sulfate, and the solvent was then distilled away under reduced pressure. The obtained residue was purified by silica gel column chromatography (eluent: chloroform-methanol), so as to obtain methyl 1-(methylsulfonyl)-1H-pyrrolo[3,2-b]pyridine-5-carboxylate (135 mg) in the form of a slight yellow solid.

### Production Example 57

While the internal temperature was kept at −63° C. or lower, diisobutylalminum hydride (1 M toluene solution; 3.2 ml) was added dropwise to a mixture of methyl 1-(methylsulfonyl)-1H-pyrrolo[3,2-b]pyridine-5-carboxylate (Production Example 56; 135 mg) and THF (16.8 ml), and the obtained mixture was then stirred at −78° C. for 1 hour.

Thereafter, the reaction mixture was poured into a mixture of water, a 1 M sodium hydroxide aqueous solution, and a saturated saline, and the thus obtained mixture was then stirred at room temperature for 10 minutes. After that, a generated product was extracted with ethyl acetate. The extract was dried over anhydrous sodium sulfate, and the solvent was then distilled away under reduced pressure. The obtained residue was purified by silica gel column chromatography (eluent: chloroform-methanol), so as to obtain 1-(methylsulfonyl)-1H-pyrrolo[3,2-b]pyridine-5-carbaldehyde (113 mg) in the form of a colorless solid.

### Production Example 58

While the internal temperature was kept at 1° C. or lower on an ice-methanol bath, a 30% sodium hydroxide aqueous solution (1.42 g) and 4-methylbenzenesulfonyl chloride (1.53 g) were successively added to a mixture of (3-methoxyoxetan-3-yl)methanol (Tetrahedron Letters, 55(30), 4117-4119, 2014; 631 mg), tetrabutylammonium iodide (124 mg), and toluene (7 ml), and the obtained mixture was then stirred at 0° C. for 1 hour, and then, at room temperature for 29 hours. Thereafter, ice water was added to the reaction mixture, and a generated product was then extracted with dichloromethane. After that, the organic layer was dried over anhydrous sodium sulfate, and the solvent was then distilled away under reduced pressure. The obtained residue was purified by silica gel column chromatography (eluent: hexane-ethyl acetate), so as to obtain (3-methoxyoxetan-3-yl)methyl 4-methylbenzenesulfonate (1.267 g) in the form of a light yellow needle crystal.

### Production Example 59

Methanesulfonic acid anhydride (240 mg) was added to a mixture of (2-methylquinolin-6-yl)methanamine (manufactured by Enamine; 209 mg), dichloromethane (4 ml), and triethylamine (0.24 ml) under ice cold, and the obtained mixture was then stirred at room temperature for 17 hours. Thereafter, ethyl acetate was added to the reaction mixture, and the thus obtained mixture was successively washed with water and a saturated saline.

The organic layer was dried over anhydrous sodium sulfate, and the solvent was then distilled away under reduced pressure. The obtained residue was purified by silica gel column chromatography (eluent: chloroform-methanol), so as to obtain N-((2-methylquinolin-6-yl)methyl)methanesulfonamide (182 mg) in the form of a light yellow solid.

### Production Example 60

Methanesulfonyl chloride (0.042 ml) was added to a mixture of N-((2-methylquinolin-6-yl)methyl)tetrahydro-2H-pyran-4-amine (manufactured by Aurora Fine Chemicals; 116 mg), dichloromethane (2.3 ml), and triethylamine (0.0946 ml) under ice cold, and the obtained mixture was then stirred at room temperature for 1 hour 30 minutes.

Thereafter, ethyl acetate was added to the reaction mixture, and then, the obtained mixture was successively washed with water, a saturated sodium hydrogen carbonate aqueous solution, and a saturated saline. After that, the organic layer was dried over anhydrous sodium sulfate, and the solvent was then distilled away under reduced pressure. The obtained residue was purified by silica gel column chromatography (eluent: chloroform-methanol), so as to obtain N-((2-methylquinolin-6-yl)methyl)-N-(tetrahydro-2H-pyran-4-yl)methanesulfonamide (122 mg) in the form of a colorless solid.

### Production Example 61

70% mCPBA (169 mg) was added to a mixture of (5-methyl-1H-pyrrolo[3,2-b]pyridin-2-yl)(morpholino)methanone (Production Example 44; 168 mg) and chloroform (5 ml) under ice cold, and the obtained mixture was then stirred at room temperature for 23 hours.

Thereafter, the reaction mixture was purified by silica gel column chromatography (eluent: chloroform-methanol), so as to obtain 5-methyl-2-(morpholine-4-carbonyl)-1H-pyrrolo[3,2-b]pyridine 4-oxide (205 mg) in the form of a light brown foam product.

### Production Example 62

Using (5-methylfuro[3,2-b]pyridin-2-yl)(morpholino)methanone (Production Example 20), dichloromethane, and 70% mCPBA, 5-methyl-2-(morpholine-4-carbonyl)furo[3,2-b]pyridine 4-oxide was obtained in the form of a colorless solid in the same manner as that of Production Example 61.

### Production Example 64

A mixture of 5-methyl-2-(morpholine-4-carbonyl)-1H-pyrrolo[3,2-b]pyridine 4-oxide (Production Example 61; 196 mg) and acetic anhydride (10 ml) was stirred at 120° C. for 3 hours, and the solvent was then removed from the reaction mixture under a nitrogen flow.

Thereafter, ethanol (10 ml) and a 1 M sodium hydroxide aqueous solution (10 ml) were added to the obtained residue, and the obtained mixture was then stirred at room temperature for 5 hours. After that, the solvent was distilled away from the reaction mixture under reduced pressure. Subsequently, toluene was added to the obtained residue, and the solvent was then distilled away again under reduced pressure. The obtained residue was purified by silica gel column chromatography (eluent: chloroform-methanol), so as to obtain (5-(hydroxymethyl)-1H-pyrrolo[3,2-b]pyridin-2-yl)(morpholino)methanone (43 mg) in the form of a light yellow solid.

### Production Example 65

A mixture of 5-methyl-2-(morpholine-4-carbonyl)furo[3,2-b]pyridine 4-oxide (Production Example 62; 106 mg) and acetic anhydride (1 ml) was stirred at 110° C. for 20 minutes, and the solvent was then removed from the reaction mixture under a nitrogen flow.

Thereafter, methanol (2 ml) and potassium carbonate (112 mg) were added to the obtained residue, and the obtained mixture was then stirred at room temperature for 30 minutes. Thereafter, water was added to the reaction mixture, and a generated product was then extracted with ethyl acetate. The organic layer was dried over anhydrous sodium sulfate, and the solvent was then distilled away under reduced pressure. The obtained residue was purified by silica gel column chromatography (eluent: chloroform-methanol), so as to obtain (5-(hydroxymethyl)furo[3,2-b]pyridin-2-yl)(morpholino)methanone (65 mg) in the form of a light yellow solid.

### Production Example 66

Phosphorus oxychloride (3.15 ml) was added to a mixture of 6-(ethoxycarbonyl)-2-methylquinoline 1-oxide (Production Example 63; 563 mg) and dichloromethane (11.3 ml) under ice cold, and the thus obtained mixture was then stirred at room temperature for 18 hours, and then, at 50° C. for 1 hour 30 minutes.

Thereafter, the solvent was distilled away from the reaction mixture under reduced pressure, and a saturated sodium hydrogen carbonate aqueous solution and a 2 M sodium hydroxide aqueous solution were then added to the obtained residue. A generated product was extracted with ethyl acetate. The organic layer was dried over anhydrous sodium sulfate, and the solvent was then distilled away under reduced pressure. The obtained residue was successively purified by silica gel column chromatography (eluents: hexane-ethyl acetate, and then, chloroform-methanol), so as to obtain ethyl 4-chloro-2-methylquinoline-6-carboxylate (322 mg) in the form of a light brown solid.

### Production Example 67

While 37% formalin (0.69 ml) and sodium triacetoxyborohydride (1.315 g) were added, in 5 equal portions each, to a mixture of N-((2-methylquinolin-6-yl)methyl)tetrahydro-2H-pyran-4-amine (manufactured by Aurora Fine Chemicals; 159 mg) and dichloromethane (5.6 ml), the mixture was stirred at room temperature for 3 days.

Thereafter, a saturated sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure.

The obtained residue was purified by silica gel column chromatography (eluent: chloroform-methanol), so as to obtain N-methyl-N-((2-methylquinolin-6-yl)methyl)tetrahydro-2H-pyran-4-amine (140 mg) in the form of a slight yellow solid.

### Production Example 68

A mixture of 2-methylbenzo[d]thiazol-6-carbaldehyde (manufactured by BLD Pharmatech; 201 mg), tetrahydro-2H-pyran-4-amine (manufactured by Apollo Scientific; 0.129 ml), and toluene (6.8 ml) was stirred under heat reflux for 6 hours, and the solvent was then distilled away from the reaction mixture under reduced pressure.

Thereafter, sodium borohydride (86 mg) was added to a mixture of the obtained residue and methanol (4 ml) under ice cold, and the thus obtained mixture was then stirred at room temperature for 6 hours 30 minutes. After that, a saturated sodium hydrogen carbonate aqueous solution was added to the reaction mixture, and a generated product was then extracted with chloroform.

The organic layer was dried over anhydrous sodium sulfate, and the solvent was then distilled away under reduced pressure. The obtained residue was purified by silica gel column chromatography (eluent: chloroform-methanol), so as to obtain N-((2-methylbenzo[d]thiazol-6-yl)methyl)tetrahydro-2H-pyran-4-amine (270 mg) in the form of a light yellow oily substance.

### Production Example 70

TFA (1 ml) was added to a mixture of tert-butyl (1-(oxetan-3-yl)piperidin-4-yl)carbamate (WO2019210828; 100 mg) and dichloromethane (1 ml) under ice cold, and the obtained mixture was then stirred at 0° C. for 2 hours 30 minutes. Thereafter, the solvent was distilled away from the reaction mixture under reduced pressure.

Thereafter, toluene was added to the obtained residue, and the solvent was then distilled away again under reduced pressure. To the obtained residue, 2-methylquinoline-6-carbaldehyde (manufactured by KANTO CHEMICAL CO., INC.; 67 mg), toluene (5 ml), and triethylamine (0.163 ml) were added, and the thus obtained mixture was then stirred under heat reflux for 6 hours. Thereafter, the solvent was distilled away from the reaction mixture under reduced pressure, and sodium borohydride (30 mg) was then added to a mixture of the obtained residue and methanol (2 ml) under ice cold. Subsequently, the obtained mixture was stirred at room temperature for 5 hours. Thereafter, the reaction mixture was treated in the same manner as that of Production Example 68, so as to obtain N-((2-methylquinolin-6-yl)methyl)-1-(oxetan-3-yl)piperidin-4-amine (46 mg) in the form of a light yellow oily substance.

### Production Example 71

TFA (2 ml) was added to a mixture of tert-butyl 4-(4-([1,1′-biphenyl]-2-yl)-2-methylquinoline-6-carbonyl)piperazine-1-carboxylate (Production Example 22; 936 mg) and dichloromethane (5 ml), and the obtained mixture was then stirred at room temperature for 2 hours 30 minutes. Thereafter, the solvent was distilled away from the reaction mixture under reduced pressure. To the obtained residue, a saturated sodium hydrogen carbonate aqueous solution, sodium hydrogen carbonate, and common salt were successively added, and a generated product was then extracted with chloroform.

The organic layer was dried over anhydrous sodium sulfate, and the solvent was then distilled away under reduced pressure, so as to obtain (4-([1,1′-biphenyl]-2-yl)-2-methylquinolin-6-yl)(piperazin-1-yl)methanone (884 mg) in the form of a slight yellow foam product.

### Production Example 72

A mixture of (2-methylquinolin-6-yl)(piperazin-1-yl)methanone (manufactured by Aurora Fine Chemicals; 484 mg), oxetan-3-one (manufactured by Tokyo Chemical Industry Co., Ltd.; 0.146 ml), DCE (9.7 ml), and acetic acid (1.09 ml) was stirred at room temperature for 3 hours, and sodium triacetoxyborohydride (804 mg) was then added to the reaction mixture under ice cold. Thereafter, the thus obtained mixture was stirred at room temperature for 1 hour 30 minutes.

Thereafter, a saturated sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure.

The obtained residue was purified by silica gel column chromatography (eluent: chloroform-methanol), so as to obtain (2-methylquinolin-6-yl)(4-(oxetan-3-yl)piperazin-1-yl)methanone (277 mg) in the form of a light yellow solid.

### Production Example 73

Using 2-methylquinoline-6-carbaldehyde (manufactured by KANTO CHEMICAL CO., INC.), 1-(oxetan-3-yl)piperazine bis(2,2,2-trifluoroacetate) (manufactured by Enamine), triethylamine, DCE, acetic acid, and sodium triacetoxyborohydride, 2-methyl-6-((4-(oxetan-3-yl)piperazin-1-yl)methyl)quinoline was obtained in the form of a light yellow solid in the same manner as that of Production Example 72.

### Production Example 74

A mixture of (4-([1,1′-biphenyl]-2-yl)-2-methylquinolin-6-yl)(piperazin-1-yl)methanone (Production Example 71; 161 mg), oxetan-3-one (manufactured by Tokyo Chemical Industry Co., Ltd.; 0.038 ml), dichloromethane (3.2 ml), and acetic acid (0.226 ml) was stirred at room temperature for 2 hours, and sodium triacetoxyborohydride (167 mg) was then added to the reaction mixture under ice cold. Thereafter, the obtained mixture was stirred at room temperature for 17 hours. Thereafter, a saturated sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure. The obtained residue was purified by silica gel column chromatography (eluent: hexane-chloroform), so as to obtain (4-([1,1′-biphenyl]-2-yl)-2-methylquinolin-6-yl)(4-(oxetan-3-yl)piperazin-1-yl)methanone (160 mg) in the form of a slight yellow oily substance.

### Production Example 75

A mixture of (2-methylquinolin-6-yl)methanamine (manufactured by Enamine; 200 mg), 2,6-dimethyltetrahydro-4H-pyran-4-one (manufactured by Combi-Blocks; 0.17 ml), DCE (4 ml), and acetic acid (0.665 ml) was stirred at room temperature for 6 hours, and sodium triacetoxyborohydride (492 mg) was then added to the reaction mixture under ice cold sodium. Thereafter, the obtained mixture was stirred at room temperature for 19 hours. Thereafter, a sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure.

The obtained residue was purified by silica gel column chromatography (eluent: chloroform-methanol), and the previously eluted low-polarity (2SR,6SR)-2,6-dimethyl-N-((2-methylquinolin-6-yl)methyl)tetrahydro-2H-pyran-4-amine (121 mg) was obtained in the form of a light yellow solid.

### Production Example 76

In the silica gel column chromatography, in which the compound of Production Example 75 was obtained, the subsequently eluted high-polarity (2SR,6RS)-2,6-dimethyl-N-((2-methylquinolin-6-yl)methyl)tetrahydro-2H-pyran-4-amine (123 mg) was obtained in the form of a light yellow solid.

The relative positions of the methyl groups in Production Example 75 and Production Example 76 were determined by comparing the chemical shift values of the two methine carbons in the pyran rings. Specifically, in Production Example 75 (50.5, 67.7 ppm), the methyl group is shifted to a higher magnetic field due to the gamma-gauche effect than in Production Example 76 (53.5, 72.0 ppm).

From the above, Production Example 76 was determined to be a (2SR,6RS) form, in which both of two methyl groups occupy equatorial positions, and Production Example 75 was determined to be a (2SR,6SR) form, in which one methyl group occupies an axial position and the other methyl group occupies an equatorial position.

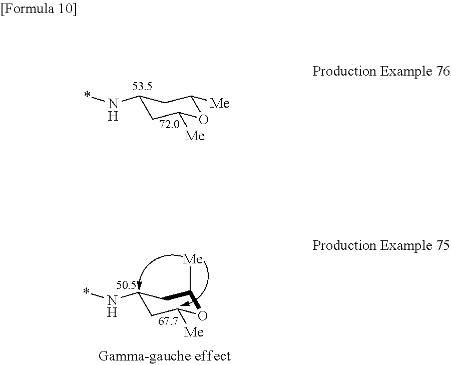

### Production Example 77

A mixture of 6-bromo-2-methylquinoline (manufactured by Combi-Blocks; 1 g), tert-butyl piperazine-1-carboxylate (manufactured by KANTO CHEMICAL CO., INC.; 1.01 g), toluene (20 ml), BINAP (280 mg), and sodium tert-butoxide (649 mg) was degassed by repeating pressure reduction and nitrogen substitution, and tris(dibenzylideneacetone)dipalladium(0) (206 mg) was then added to the reaction mixture, followed by stirring the obtained mixture at 110° C. for 24 hours.

Thereafter, ethyl acetate was added to the reaction mixture, and precipitated insoluble matters were then removed by filtration. After that, the solvent was distilled away from the filtrate under reduced pressure. The obtained residue was purified by silica gel column chromatography (eluent: chloroform-ethyl acetate), so as to obtain tert-butyl 4-(2-methylquinolin-6-yl)piperazine-1-carboxylate (952 mg) in the form of a brown solid.

### Production Example 78

A mixture of tert-butyl 4-(2-methylquinolin-6-yl)piperazine-1-carboxylate (Production Example 77; 191 mg), 1,4-dioxane (2 ml), methanol (2 ml), and a 4 M hydrogen chloride 1,4-dioxane solution (4 ml) was stirred at room temperature for 4 hours, and the solvent was then distilled away from the reaction mixture under reduced pressure. Thereafter, toluene was added to the obtained residue, and the solvent was then distilled away again under reduced pressure.

Using the obtained residue, oxetan-3-one (manufactured by Tokyo Chemical Industry Co., Ltd.; 0.066 ml), triethylamine (0.325 ml), DCE (3.9 ml), acetic acid (0.334 ml), and sodium triacetoxyborohydride (247 mg), 2-methyl-6-(4-(oxetan-3-yl)piperazin-1-yl)quinoline (99 mg) was obtained in the form of a light yellow solid in the same manner as that of Production Example 72.

### Production Example 79

A mixture of 2-methylbenzo[d]thiazole-5-carbaldehyde (manufactured by BLD Pharmatech; 117 mg), methanesulfonamide (75 mg), toluene (2.4 ml), and titanium(IV) ethoxide (0.152 ml) was stirred at 110° C. for 6 hours.

Thereafter, the solvent was distilled away from the reaction mixture under reduced pressure, and methanol (1.2 ml) and THF (1.2 ml) were then added to the obtained residue. Subsequently, sodium borohydride (50 mg) was added to the obtained mixture under ice cold, and the thus obtained mixture was then stirred at room temperature for 1 hour 30 minutes.

Thereafter, the solvent was distilled away from the reaction mixture under reduced pressure, and a saturated sodium hydrogen carbonate aqueous solution and chloroform were added to the obtained residue. After that, precipitated insoluble matters were removed by filtration. The filtrate was extracted with chloroform, the organic layer was then dried over anhydrous sodium sulfate, and the solvent was then distilled away under reduced pressure. The obtained residue was purified by silica gel column chromatography (eluent: chloroform-methanol), so as to obtain N-((2-methylbenzo[d]thiazol-5-yl)methyl)methanesulfonamide (153 mg) in the form of a light yellow solid.

### Production Example 81

A mixture of (2-methylquinolin-6-yl)(piperazin-1-yl)methanone (manufactured by Aurora Fine Chemicals; 376 mg), ethanol (7.6 ml), potassium carbonate (305 mg), and 2-(methoxymethyl)oxirane (manufactured by Tokyo Chemical Industry Co., Ltd.; 0.157 ml) was stirred at 80° C. for 3 hours. Thereafter, 2-(methoxymethyl)oxirane (0.0655 ml) was added to the reaction mixture, and the thus obtained mixture was stirred 80° C. for 1 hour.

Subsequently, the solvent was distilled away from the reaction mixture under reduced pressure, and toluene was then added to the obtained residue. After that, the solvent was distilled away again under reduced pressure. The obtained residue was purified by silica gel column chromatography (eluent: chloroform-methanol), so as to obtain (4-(2-hydroxy-3-methoxypropyl)piperazin-1-yl)(2-methylquinolin-6-yl)methanone (376 mg) in the form of a light yellow oily substance.

### Production Example 83

A mixture of ethyl 4-chloro-2-methylquinoline-6-carboxylate (Production Example 66; 145 mg), DMF (6.1 ml), and sodium azide (189 mg) was stirred under a nitrogen atmosphere at 120° C. for 6 hours, and the solvent was then distilled away from the reaction mixture under reduced pressure. Ice water was added to the obtained residue, and a generated product was then extracted with ethyl acetate. Thereafter, the organic layer was dried over anhydrous sodium sulfate, and the solvent was then distilled away under reduced pressure. The obtained residue was purified by silica gel column chromatography (eluent: hexane-chloroform), so as to obtain ethyl 4-amino-2-methylquinoline-6-carboxylate (48 mg) in the form of a light brown solid.

### Production Example 84

A mixture of ethyl 4-chloro-2-methylquinoline-6-carboxylate (Production Example 66; 102 mg), DMF (4.3 ml), and sodium azide (133 mg) was stirred under a nitrogen atmosphere at 120° C. for 1 hour 30 minutes. Thereafter, ice water was added to the reaction mixture, and the thus obtained mixture was then stirred at room temperature for 1 hour. Subsequently, a generated product was extracted with ethyl acetate, and the organic layer was then dried over anhydrous sodium sulfate. After that, the solvent was distilled away under reduced pressure.

The obtained residue was purified by silica gel column chromatography (eluent: chloroform-methanol), so as to obtain ethyl 4-azido-2-methylquinoline-6-carboxylate (90 mg) in the form of a light brown solid.

### Production Example 85

A mixture of 2-methylquinoline-6-carboxylic acid (manufactured by Tokyo Chemical Industry Co., Ltd.; 770 mg), DMF (6 ml), and 1,1′-carbonyldiimidazole (670 mg) was stirred at 40° C. for 1 hour. Thereafter, tert-butanol (0.787 ml) and 1,8-diazabicyclo[5.4.0]undecene (0.62 ml) were added to the reaction mixture, and the thus obtained mixture was then stirred at 80° C. for 4 hours. Thereafter, water was added to the reaction mixture, and a generated product was then extracted with tert-butyl methyl ether. Subsequently, the organic layer was dried over anhydrous sodium sulfate, and the solvent was then distilled away under reduced pressure.

The obtained residue was purified by silica gel column chromatography (eluent: chloroform), so as to obtain tert-butyl 2-methylquinoline-6-carboxylate (733 mg) in the form of a yellow solid.

### Production Example 87

A mixture of ethyl 4-chloro-2-methylquinoline-6-carboxylate (Production Example 66, 137 mg), DMF (1.4 ml), zin cyanide (129 mg), and triphenylphosphine (63 mg) was stirred under microwave irradiation at 160° C. for 30 minutes. Thereafter, the solvent was removed from the reaction mixture under a nitrogen flow, and the obtained residue was then purified by silica gel column chromatography (eluent: hexane-ethyl acetate), so as to obtain ethyl 4-cyano-2-methylquinoline-6-carboxylate (110 mg) in the form of a slight brown solid.

### Production Example 89

A mixture of 1-acetyl-2-(3-methoxy-4-(2-morpholino-2-oxoethoxy)benzylidene)indolin-3-one (Example 2; 129 mg), methanol (13 ml), and potassium carbonate (123 mg) was stirred at room temperature for 1 hour 30 minutes. Thereafter, chloroform and water were added to the reaction mixture, and a generated product was then extracted with chloroform. After that, the organic layer was dried over anhydrous sodium sulfate, and the solvent was then distilled away under reduced pressure.

The obtained residue was purified by silica gel column chromatography (eluent: chloroform-methanol), so as to obtain 2-(3-methoxy-4-(2-morpholino-2-oxoethoxy)benzylidene)indolin-3-one (125 mg) in the form of an orange foam product..

### Production Example 90

A mixture of ethyl 4-azido-2-methylquinoline-6-carboxylate (Production Example 84; 36 mg), ethanol (1 ml), and a 1 M sodium hydroxide aqueous solution (0.281 ml) was stirred at room temperature for 5 hours 30 minutes.

Thereafter, 1 M hydrochloric acid (0.281 ml) was added to the reaction mixture, and the solvent was then distilled away under reduced pressure. After that, toluene was added to the obtained residue, and the solvent was then distilled away again under reduced pressure, so as to obtain crude 4-azido-2-methylquinoline-6-carboxylic acid in the form of a light brown solid.

### Production Example 93

A mixture of ethyl 4-amino-2-methylquinoline-6-carboxylate (Production Example 83; 48 mg) and 6 M hydrochloric acid (2 ml) was stirred at 100° C. for 26 hours, and thereafter, the solvent was distilled away from the reaction mixture under reduced pressure.

Thereafter, toluene was added to the obtained residue, and the solvent was then distilled away again under reduced pressure, so as to obtain 4-amino-2-methylquinoline-6-carboxylic acid dihydrochloride (51 mg) in the form of a light brown solid.

### Production Example 94

A mixture of ethyl 4-([1,1′-biphenyl]-2-yl)-2-methylquinoline-6-carboxylate (Production Example 106; 1.568 g), ethanol (16 ml), THF (8 ml), and a 1 M sodium hydroxide aqueous solution (5.2 ml) was stirred at room temperature for 3 hours, and a 1 M sodium hydroxide aqueous solution (2.6 ml) was then added to the reaction mixture, followed by stirring the obtained mixture at room temperature for 19 hours.

Thereafter, water was added to the reaction solution, and ethanol and THF were then distilled away under reduced pressure, followed by washing the residue with chloroform.

Subsequently, 1 M hydrochloric acid (7.8 ml) was added to the obtained solution, and the thus obtained mixture was then stirred at room temperature for 22 hours. After that, the obtained solid was collected by filtration, and was successively washed with water and hexane, so as to obtain 4-([1,1′-biphenyl]-2-yl)-2-methylquinoline-6-carboxylic acid (1.317 g) in the form of slight brown powders.

### Production Example 95

A mixture of (4-chloro-2-methylquinolin-6-yl)(morpholino)methanone (Production Example 34; 102 mg), phenylboronic acid (51 mg), butanol (2 ml), a 1.2 M cesium carbonate aqueous solution (0.497 ml), and X-Phos (20 mg) was degassed by repeating pressure reduction and nitrogen substitution, and palladium acetate (8 mg) was then added to the reaction mixture, followed by stirring the obtained mixture at room temperature for 1 hour.

Thereafter, the solvent was removed from the reaction mixture under a nitrogen flow, and ethyl acetate was then added to the obtained residue, followed by washing the obtained mixture with a saturated saline.

The organic layer was dried over anhydrous sodium sulfate, and the solvent was then distilled away under reduced pressure. The obtained residue was purified by silica gel column chromatography (eluent: chloroform-methanol), so as to obtain (2-methyl-4-phenylquinolin-6-yl)(morpholino)methanone (97 mg) in the form of a light yellow oily substance.

### Production Example 96

A mixture of (4-chloro-2-methylquinolin-6-yl)(morpholino)methanone (Production Example 34; 81 mg), 3-pyridylboronic acid (41 mg), butanol (1.6 ml), a 1.2 M cesium carbonate aqueous solution (0.395 ml), and X-Phos (16 mg) was degassed by repeating pressure reduction and nitrogen substitution, and palladium acetate (6 mg) was then added to the reaction mixture. The thus obtained mixture was stirred at room temperature for 1 hour, and then, at 80° C. for 3 hours.

Thereafter, the solvent was removed from the reaction mixture under a nitrogen flow, and ethyl acetate was then added to the obtained residue, followed by washing the obtained mixture with a saturated saline. The organic layer was dried over anhydrous sodium sulfate, and the solvent was then distilled away under reduced pressure. The obtained residue was purified by silica gel column chromatography (eluent: chloroform-methanol), so as to obtain the previously eluted low-polarity (4-butoxy-2-methylquinolin-6-yl)(morpholino)methanone (37 mg) in the form of a colorless oily substance.

### Production Example 97

In the silica gel column chromatography, in which the compound of Production Example 96 was obtained, the subsequently eluted high-polarity (2-methyl-4-(pyridin-3-yl)quinolin-6-yl)(morpholino)methanone (36 mg) was obtained in the form of a colorless oily substance.

### Production Example 98

A mixture of (4-chloro-2-methylquinolin-6-yl)(morpholino)methanone (Production Example 34; 65 mg), [1,1′-biphenyl]-2-ylboronic acid (53 mg), 1,2-dimethoxyethane (1.6 ml), a 3 M sodium carbonate aqueous solution (0.224 ml), and a [1,1′-bis(diphenylphosphino)ferrocene]dichloropalladium(II) dichloromethane adduct (18 mg) was stirred under a nitrogen atmosphere at 90° C. for 6 hours, and then, at room temperature for 5 days.

Thereafter, the reaction mixture was purified by silica gel column chromatography (eluent: chloroform-methanol), so as to obtain (4-([1,1′-biphenyl]-2-yl)-2-methylquinolin-6-yl)(morpholino)methanone (93 mg) in the form of a light brown oily substance.

### Production Example 107

A methanol solution (4 ml) of 4-Chloro-6-(morpholine-4-carbonyl)quinoline-2-carbaldehyde (Production Example 153; 399 mg) was added to a mixture of sodium borohydride (67 mg) and methanol (4 ml) under ice cold, and the obtained mixture was then stirred at 0° C. for 30 minutes.

Thereafter, water was added to the reaction mixture, and methanol was then distilled away under reduced pressure. After that, a generated product was extracted with chloroform. The extract was dried over anhydrous sodium sulfate, and the solvent was then distilled away under reduced pressure. The obtained residue was purified by silica gel column chromatography (eluent: chloroform-methanol), so as to obtain (4-chloro-2-(hydroxymethyl)quinolin-6-yl)(morpholino)methanone (227 mg) in the form of a colorless solid.

### Production Example 108

A mixture of (4-chloro-2-(hydroxymethyl)quinolin-6-yl)(morpholino)methanone (Production Example 107; 168 mg), DMF (1.7 ml), tert-butyl dimethylchlorosilane (99 mg), and imidazole (93 mg) was stirred at room temperature for 4 hours 30 minutes.

Thereafter, the solvent was removed from the reaction mixture under a nitrogen flow, and the obtained residue was then purified by silica gel column chromatography (eluent: hexane-chloroform-methanol), so as to obtain (2-(((tert-butyl dimethylsilyl)oxy)methyl)-4-chloroquinolin-6-yl)(morpholino)methanone (205 mg) in the form of a light blue solid.

### Production Example 109

A mixture of (4-chloro-2-methylquinolin-6-yl)(morpholino)methanone (Production Example 34; 151 mg), piperidine-4-carbonitrile (manufactured by Enamine; 0.0927 ml), toluene (3 ml), BINAP (129 mg), cesium carbonate (254 mg), and palladium acetate (23 mg) was stirred under a nitrogen atmosphere at 100° C. for 18 hours.

Thereafter, the reaction mixture was purified by silica gel column chromatography (eluent: chloroform-methanol), so as to obtain 1-(2-methyl-6-(morpholine-4-carbonyl)quinolin-4-yl)piperidine-4-carbonitrile (160 mg) in the form of a light brown solid.

### Production Example 111

A mixture of 1-(4-(2-(((tert-butyl dimethylsilyl)oxy)methyl)-6-(morpholine-4-carbonyl)quinolin-4-yl)piperazin-1-yl)ethan-1-one (Production Example 110; 103 mg) and tetrabutylammonium fluoride (1 M THF solution; 0.5 ml) was stirred at room temperature for 30 minutes, and the solvent was then distilled away from the reaction mixture under reduced pressure.

Thereafter, water was added to the obtained residue, and a generated product was then extracted with dichloromethane. After that, the extract was dried over anhydrous sodium sulfate, and the solvent was then distilled away under reduced pressure. The obtained residue was purified by silica gel column chromatography (eluent: chloroform-methanol), so as to obtain 1-(4-(2-(hydroxymethyl)-6-(morpholine-4-carbonyl)quinolin-4-yl)piperazin-1-yl)ethan-1-one (67 mg) in the form of a light yellow oily substance.

### Production Example 112

A mixture of N-((2-methylquinolin-6-yl)methyl)tetrahydro-2H-pyran-4-amine (manufactured by Aurora Fine Chemicals; 99 mg), dichloromethane (4 ml), and di-tert-butyl dicarbonate (146 mg) was stirred at room temperature for 3 hours, and the solvent was then distilled away from the reaction mixture under reduced pressure.

The obtained residue was purified by silica gel column chromatography (eluent: chloroform-methanol), so as to obtain tert-butyl ((2-methylquinolin-6-yl)methyl)(tetrahydro-2H-pyran-4-yl)carbamate (112 mg) in the form of a slight yellow oily substance.

### Production Example 119

A mixture of (2-methylquinolin-6-yl)(morpholino)methanone (Production Example 19; 527 mg), 1,4-dioxane (10 ml), and selenium dioxide (251 mg) was stirred at 80° C. for 3 hours, and an insoluble matter in the reaction mixture was then removed by filtration. Thereafter, the solvent was distilled away from the filtrate under reduced pressure.

The obtained residue was purified by silica gel column chromatography (eluent: chloroform-methanol), so as to obtain 6-(morpholine-4-carbonyl)quinoline-2-carbaldehyde (434 mg) in the form of a light brown solid.

### Production Example 181

A mixture of N-(2-methyl-6-(morpholine-4-carbonyl)quinolin-4-yl)acetamide (Production Example 50; 52 mg), 1,4-dioxane (1 ml), and selenium dioxide (37 mg) was stirred at 80° C. for 4 hours, and DMF (0.5 ml) was then added to the reaction mixture, followed by stirring the obtained mixture at 80° C. for 2 hours.

Thereafter, the solvent was removed from the reaction mixture under a nitrogen flow, and the obtained residue was then purified by silica gel column chromatography (eluent: chloroform-methanol), so as to obtain N-(2-formyl-6-(morpholine-4-carbonyl)quinolin-4-yl)acetamide (27 mg) in the form of a slight brown solid.

### Production Example 182

A mixture of 2-methyl-6-(morpholine-4-carbonyl)quinoline-4-carboxamide (Production Example 32; 35 mg), 1,4-dioxane (1.4 ml), and selenium dioxide (30 mg) was stirred at 80° C. for 2 hours, and an insoluble matter in the reaction mixture was then removed by filtration. After that, the solvent was distilled away from the filtrate under reduced pressure.

The obtained residue was pulverized in a mixed solvent of methanol and chloroform to obtain crude 2-formyl-6-(morpholine-4-carbonyl)quinoline-4-carboxamide in the form of a light brown solid.

### Production Example 185

A mixture of (2-methylthiazol-4-yl)(morpholino)methanone (manufactured by Aurora Fine Chemicals; 75 mg), 1,4-dioxane (1.5 ml), and selenium dioxide (40 mg) was stirred under microwave irradiation at 150° C. for 20 minutes, and selenium dioxide (118 mg) was then added to the reaction mixture, followed by stirring the obtained mixture under microwave irradiation at 150° C. for 8 hours. Thereafter, an insoluble matter in the reaction mixture was removed by filtration, and the solvent was then distilled away from the filtrate under reduced pressure. The obtained residue was purified by silica gel column chromatography (eluent: chloroform-methanol), so as to obtain 4-(morpholine-4-carbonyl)thiazole-2-carbaldehyde (28 mg) in the form of a light yellow oily substance.

### Production Example 188

A mixture of (6-(hydroxymethyl)naphthalen-2-yl)(morpholino)methanone (Production Example 36; 184 mg), chloroform (2.7 ml), and manganese dioxide (460 mg) was stirred at 60° C. for 2 hours 30 minutes, and an insoluble matter in the reaction mixture was then removed by filtration. After that, the solvent was distilled away from the filtrate under reduced pressure. The obtained residue was purified by silica gel column chromatography (eluent: hexane-chloroform-methanol), so as to obtain 6-(morpholine-4-carbonyl)-2-naphthalene aldehyde (164 mg) in the form of a colorless solid.

### Production Example 190

Using (5-(hydroxymethyl)-1H-pyrrolo[3,2-b]pyridin-2-yl)(morpholino)methanone (Production Example 64), 2-propanol, and manganese dioxide, 2-(morpholine-4-carbonyl)-1H-pyrrolo[3,2-b]pyridine-5-carbaldehyde was obtained in the form of a colorless solid in the same manner as that of Production Example 188.

### Production Example 192

A mixture of (2-(hydroxymethyl)-1H-indol-5-yl)(morpholino)methanone (Production Example 40; 100 mg), dichloromethane (1 ml), and manganese dioxide (167 mg) was stirred at room temperature for 6 hours 30 minutes, and the reaction mixture was then purified by silica gel column chromatography (eluent: chloroform-methanol), so as to obtain 5-(morpholine-4-carbonyl)-1H-indole-2-carbaldehyde (73 mg) in the form of a light yellow solid.

### Production Example 194

A mixture of 6-(2-((tetrahydro-2H-pyran-2-yl)oxy)ethoxy)quinoline-2-carbaldehyde (Production Example 166; 287 mg), 1-acetylindolin-3-one (manufactured by Combi-Blocks; 167 mg), toluene (5 ml), Molecular Sieve 4A (1 g), and piperidine (0.0188 ml) was stirred at 80° C. for 3 hours, and the reaction mixture was then purified by silica gel column chromatography (eluent: hexane-ethyl acetate), so as to obtain (Z)-1-acetyl-2-((6-(2-((tetrahydro-2H-pyran-2-yl)oxy)ethoxy)quinolin-2-yl)methylene)indolin-3-one (227 mg) in the form of a brown oily substance.

### Production Example 199

A mixture of tert-butyl 2-formylquinoline-6-carboxylate (Production Example 164; 171 mg), 1-acetylindolin-3-one (manufactured by Combi-Blocks; 140 mg), toluene (5 ml), Molecular Sieve 4A (1 g), and piperidine (0.0131 ml) was stirred at 80° C. for 3 hours, and the solvent was then distilled away from the reaction mixture under reduced pressure. The obtained residue was successively purified by silica gel column chromatography (eluent: hexane-ethyl acetate) and then, by gel permeation chromatography (eluent: chloroform), so as to obtain tert-butyl (Z)-2-((1-acetyl-3-oxoindolin-2-ylidene)methyl)quinoline-6-carboxylate (191 mg) in the form of a light brown oily substance.

### Production Example 205

A mixture of tert-butyl ((2-formylbenzo[d]thiazol-5-yl)methyl)(tetrahydro-2H-pyran-4-yl)carbamate (Production Example 167; 120 mg), 1-acetylindolin-3-one (manufactured by Combi-Blocks; 56 mg), toluene (3 ml), Molecular Sieve 4A (1 g), and piperidine (0.0063 ml) was stirred at 80° C. for 3 hours, and the reaction mixture was then purified by silica gel column chromatography (eluent: chloroform-methanol).

The previously eluted low-polarity fraction was collected, and the solvent was then distilled away under reduced pressure. The obtained residue was purified by gel permeation chromatography (eluent: chloroform), so as to obtain tert-butyl (Z)-((2-((1-acetyl-3-oxoindolin-2-ylidene)methyl)benzo[d]thiazol-5-yl)methyl)(tetrahydro-2H-pyran-4-yl)carbamate (22 mg) in the form of a yellow oily substance.

### Production Example 206

In the silica gel column chromatography, in which the compound of Production Example 205 was obtained, the subsequently eluted high-polarity fraction was collected, and the solvent was then distilled away under reduced pressure. The obtained residue was purified by gel permeation chromatography (eluent: chloroform), so as to obtain tert-butyl (E)-((2-((1-acetyl-3-oxoindolin-2-ylidene)methyl)benzo[d]thiazol-5-yl)methyl)(tetrahydro-2H-pyran-4-yl)carbamate (23 mg) in the form of a yellow solid.

### Production Example 209

Oxalyl chloride (0.126 ml) was added to a mixture of 4-([1,1′-biphenyl]-2-yl)-2-methylquinoline-6-carboxylic acid (Production Example 94; 100 mg), dichloromethane (2 ml), and DMF (0.0023 ml) under ice cold, and the obtained mixture was then stirred at room temperature for 16 hours. The solvent was distilled away from the reaction mixture under reduced pressure. THF (10 ml) was added to the obtained residue, and the solvent was then distilled away under reduced pressure. After that, THF (3 ml) was added to the residue, and 28% ammonia water (0.1 ml) was then added thereto under ice cold. The thus obtained mixture was stirred at room temperature for 4 hours. Thereafter, the solvent was distilled away from the reaction mixture under reduced pressure, and the obtained residue was then purified by silica gel column chromatography (eluent: chloroform-methanol), so as to obtain 4-([1,1′-biphenyl]-2-yl)-2-methylquinoline-6-carboxamide (98 mg) in the form of a colorless solid.

### Production Example 211

A mixture of tert-butyl 6-(2-methyl-6-(morpholine-4-carbonyl)quinolin-4-yl)-3,4-dihydroisoquinoline-2-(1H)-carboxylate (Production Example 337; 819 mg), dichloromethane (4 ml), and TFA (4 ml) was stirred at room temperature for 2 hours, and a saturated sodium hydrogen carbonate aqueous solution and sodium hydrogen carbonate were then added to the reaction mixture. A generated product was extracted with chloroform. The organic layer was dried over anhydrous sodium sulfate, and the solvent was then distilled away under reduced pressure, so as to obtain (2-methyl-4-(1,2,3,4-tetrahydroisoquinolin-6-yl)quinolin-6-yl)(morpholino)methanone (767 mg) in the form of a light brown foam product.

### Production Example 212

To a mixture of 4-([1,1′-biphenyl]-2-yl)-2-methyl-N-(tetrahydro-2H-pyran-4-yl)quinoline-6-carboxamide (Production Example 400; 128 mg) and THF (13 ml), lithium aluminum hydride (115 mg) was added under a nitrogen flow, and the obtained mixture was then stirred at 70° C. for 30 minutes, and then, at room temperature for 17 hours. Thereafter, sodium sulfate hydrate, THF, and ethyl acetate were added to the reaction mixture under ice cold, and an insoluble matter was then removed by filtration. After that, the solvent was distilled away from the filtrate under reduced pressure. The obtained residue was purified by silica gel column chromatography (eluent: chloroform-methanol), so as to obtain N-((4-([1,1′-biphenyl]-2-yl)-2-methylquinolin-6-yl)methyl)tetrahydro-2H-pyran-4-amine (69 mg) in the form of a colorless oily substance.

### Production Example 214

A 1 M sodium hydroxide aqueous solution (1.34 ml) was added to a mixture of ethyl 4-(1H-indazol-4-yl)-2-methylquinoline-6-carboxylate (Production Example 338; 247 mg), ethanol (2.5 ml), and THF (1.3 ml), and the obtained mixture was then stirred at room temperature for 17 hours. Thereafter, 1 M hydrochloric acid was added to the reaction mixture, and the solvent was then distilled away under reduced pressure. Water (20 ml) was added to the obtained residue, and the obtained mixture was then stirred at room temperature for 2 hours 15 minutes. Thereafter, the obtained solid was collected by filtration, and was then dried under reduced pressure to obtain 4-(1H-indazol-4-yl)-2-methylquinoline-6-carboxylic acid (183 mg) in the form of a yellow solid.

### Production Example 220

To a mixture of (4-(1H-indazol-4-yl)-2-methylquinolin-6-yl)(morpholino)methanone (Production Example 413; 184 mg) and THF (4 ml), 60% sodium hydride (39 mg) was added under ice cold, and the obtained mixture was then stirred under ice cold for 1 hour 30 minutes. Thereafter, trityl chloride (269 mg) was added to the reaction mixture, and the thus obtained mixture was then stirred at room temperature for 20 hours. Thereafter, water was added to the reaction mixture, and then, a generated product was successively extracted with dichloromethane and chloroform. Subsequently, the organic layer was dried over anhydrous sodium sulfate, and the solvent was then distilled away under reduced pressure. The obtained residue was purified by silica gel column chromatography (eluent: chloroform-methanol), so as to obtain (2-methyl-4-(N-tritylindazol-4-yl)quinolin-6-yl)(morpholino)methanone (52 mg) in the form of a colorless solid.

### Production Example 223

In the silica gel column chromatography using hexane-ethyl acetate as an eluent upon the synthesis of the compound of Production Example 66, ethyl 3-chloro-2-methylquinoline-6-carboxylate, which had been eluted before the compound of Production Example 66, was obtained in the form of a colorless solid.

### Production Example 224

Using tert-butyl 4-([1,1′-biphenyl]-3-yl)-2-methylquinoline-6-carboxylate (Production Example 432), 1,2-dimethoxyethane, and selenium dioxide, tert-butyl 4-([1,1′-biphenyl]-3-yl)-2-formylquinoline-6-carboxylate (344.2 mg) was obtained in the form of a light orange solid in the same manner as that of Production Example 119.

### Production Example 237

A mixture of 4-(3-bromophenyl)-1-trityl-1H-pyrazole (Production Example 221; 1.687 g), 4,4,4′,4′,5,5,5′,5′-octamethyl-2,2′-bi(1,3,2-dioxabolorane) (1.17 g), 1,4-dioxane (17 ml), potassium acetate (605 mg), and a [1,1′-bis(diphenylphosphino)ferrocene]dichloropalladium(II)dichloromethane adduct (252 mg) was stirred under a nitrogen atmosphere at room temperature for 5 hours 30 minutes, and then, at 80° C. for 16 hours. Thereafter, the solvent was distilled away from the reaction mixture under reduced pressure, and toluene (20 ml) was then added to the obtained residue. After that, the solvent was distilled away under reduced pressure. The obtained residue was purified by silica gel column chromatography (eluent: hexane-ethyl acetate), so as to obtain 4-(3-(4,4,5,5-tetramethyl-1,3,2-dioxaborolan-2-yl)phenyl)-1-trityl-1H-pyrazole (1.618 g) in the form of a light yellow oily substance.

### Production Example 238

A mixture of (4-chloro-2-methylquinolin-6-yl)(morpholino)methanone (Production Example 34; 209 mg), (1-(tert-butoxycarbonyl)-1H-pyrazol-4-yl)boronic acid (manufactured by Apollo Scientific; 305 mg), 1,4-dioxane (24 ml), water (2.4 ml), cesium carbonate (703 mg), and tetrakis(triphenylphosphine)palladium(0) (42 mg) was stirred under a nitrogen atmosphere at 80° C. for 16 hours. Thereafter, the solvent was distilled away from the reaction mixture under reduced pressure, and toluene (10 ml) was then added to the obtained residue. After that, the solvent was distilled away under reduced pressure. Subsequently, dichloromethane (2 ml) and TFA (2 ml) were added to the obtained residue, and the obtained mixture was then stirred at room temperature for 1 hour 30 minutes. Thereafter, a saturated sodium hydrogen carbonate aqueous solution and sodium hydrogen carbonate were added to the reaction mixture, and then, a generated product was successively extracted with chloroform and with chloroform-isopropanol (4:1). The organic layer was dried over anhydrous sodium sulfate, and the solvent was then distilled away under reduced pressure. The obtained residue was purified by silica gel column chromatography (eluent: chloroform-methanol), so as to obtain (2-methyl-4-(1H-pyrazol-4-yl)quinolin-6-yl)(morpholino)methanone (146 mg) in the form of a light yellow foam product.

### Production Example 239

A mixture of ethyl 4-chloro-2-methylquinoline-6-carboxylate (Production Example 66; 1.46 g), propionitrile (44 ml), and bromotrimethylsilane (1.52 ml) was stirred at 100° C. for 6 hours 30 minutes. Thereafter, the reaction mixture was poured into a mixture of a 2 M sodium hydroxide aqueous solution (44 ml) and ice (110 ml), and a generated product was then extracted with diethyl ether. Thereafter, the organic layer was dried over anhydrous sodium sulfate, and the solvent was then distilled away under reduced pressure. The obtained residue was purified by silica gel column chromatography (eluent: hexane-chloroform), so as to obtain ethyl 4-bromo-2-methylquinoline-6-carboxylate (1.236 g) in the form of a light brown solid.

### Production Example 240

A mixture of ethyl 4-bromo-2-methylquinoline-6-carboxylate (Production Example 239; 58 mg), 4,4,4′,4′,5,5,5′,5′-octamethyl-2,2′-bi(1,3,2-dioxabolorane) (65 mg), 1,4-dioxane (1.2 ml), potassium acetate (29 mg), tricyclohexylphosphine (11 mg), and tris(dibenzylideneacetone)dipalladium(0) (18 mg) was stirred under a nitrogen atmosphere at 90° C. for 3 hours, and an insoluble matter in the reaction mixture was then removed by filtration. Thereafter, the solvent was distilled away from the filtrate under reduced pressure. To the obtained residue, 5-bromopyrimidine (32 mg), 1,2-dimethoxyethane (1.2 ml), a 3 M sodium carbonate aqueous solution (0.131 ml), and tetrakis(triphenylphosphine)palladium(0) (12 mg) were added, and the obtained mixture was then stirred under a nitrogen atmosphere at 90° C. for 3 hours. Subsequently, the reaction mixture was purified by silica gel column chromatography (eluent: chloroform-methanol), so as to obtain ethyl 2-methyl-4-(pyrimidin-5-yl)quinoline-6-carboxylate (49 mg) in the form of a light brown solid.

### Production Example 248

1,1,3,3-Tetramethyldisiloxane (2.6 ml) was added to a mixture of 4-([1,1′-biphenyl]-4-yl)-2-methyl-N-(tetrahydro-2H-pyran-4-yl)quinoline-6-carboxamide (Production Example 277; 1.24 g), triruthenium dodecacarbonyl (203.4 mg), and toluene (12 ml), and the obtained mixture was then stirred under an argon atmosphere at 55° C. for 8 hours. Thereafter, the solvent was distilled away from the reaction mixture under reduced pressure, and the obtained residue was then purified by silica gel column chromatography (eluent: chloroform-methanol), so as to obtain N-((4-([1,1′-biphenyl]-4-yl)-2-methylquinolin-6-yl)methyl)tetrahydro-2H-pyran-4-amine (1.51 g) in the form of a reddish black oily substance.

### Production Example 252

A mixture of tert-butyl 5-(2-methyl-6-(morpholine-4-carbonyl)quinolin-4-yl)-3,6-dihydropyridine-1(2H)-carboxylate (Production Example 358; 225 mg), 5% Pd/C PE type (hydrous; manufactured by N. E. CHEMCAT CORPORATION; 23 mg), and methanol (10 ml) was stirred under a hydrogen atmosphere of 0.3 MPa for 17 hours. Thereafter, an insoluble matter in the reaction mixture was removed by filtration, and the solvent was then distilled away from the filtrate under reduced pressure. The obtained residue was purified by gel permeation chromatography (eluent: chloroform), so as to obtain tert-butyl 3-(2-methyl-6-(morpholine-4-carbonyl)quinolin-4-yl)piperidine-1-carboxylate (157 mg) in the form of a brown oily substance.

### Production Example 253

A mixture of tert-butyl 3-(2-methyl-6-(morpholine-4-carbonyl)quinolin-4-yl)piperidine-1-carboxylate (Production Example 252; 150 mg), dichloromethane (3 ml), and TFA (1 ml) was stirred at room temperature for 4 hours, and the solvent was then distilled away from the reaction mixture under reduced pressure. After that, dichloromethane (10 ml) was added to the obtained residue, and the pH of the obtained mixture was then adjusted to pH 10 or more with triethylamine. Thereafter, acetic anhydride (70 mg) was added to the reaction mixture, and the thus obtained mixture was then stirred at room temperature for 2 hours. After that, the solvent was distilled away from the reaction mixture under reduced pressure. The obtained residue was successively purified by aminopropyl silica gel column chromatography (eluent: ethyl acetate) and then, by gel permeation chromatography (eluent: chloroform), so as to obtain 1-(3-(2-methyl-6-(morpholine-4-carbonyl)quinolin-4-yl)piperidin-1-yl)ethan-1-one (73 mg) in the form of a colorless solid.

### Production Example 255

60% Sodium hydride (164 mg) was added to a mixture of 3-bromo-1H-1,2,4-triazole (504 mg) and DMF (7.8 ml) under ice cold, and the obtained mixture was then stirred under ice cold for 30 minutes. Thereafter, (2-(chloromethoxy)ethyl)trimethylsilane (0.741 ml) was added to the reaction mixture, and the thus obtained mixture was then stirred at room temperature for 20 hours. Subsequently, water was added to the reaction mixture, and a generated product was then extracted with ethyl acetate. After that, the organic layer was successively washed with water and a saturated saline, and was then dried over anhydrous sodium sulfate. The solvent was distilled away under reduced pressure, and the obtained residue was then purified by silica gel column chromatography (eluent: hexane-ethyl acetate), so as to obtain 3-bromo-N-((2-(trimethylsilyl)ethoxy)methyl)-1,2,4-triazole (821 mg) in the form of a colorless oily substance.

### Production Example 256

Using 2-methyl-4-(1H-pyrazol-4-yl)quinoline (Production Example 360), THF, 60% sodium hydride, and (2-(chloromethoxy)ethyl)trimethylsilane, 2-methyl-4-(1-((2-(trimethylsilyl)ethoxy)methyl)-1H-pyrazol-4-yl)quinoline was obtained in a light brown oily substance in the same manner as that of Production Example 255.

### Production Example 257

Trifluoromethanesulfonic acid anhydride (0.0922 ml) was added to a mixture of (5-hydroxy-2-methylquinolin-6-yl)(morpholino)methanone (Production Example 282; 102 mg), dichloromethane (4.1 ml), and pyridine (0.0543 ml) under ice cold, and the obtained mixture was then stirred under ice cold for 3 hours. Thereafter, a saturated sodium hydrogen carbonate aqueous solution was added to the reaction mixture, and a generated product was then extracted with chloroform. After that, the organic layer was dried over anhydrous sodium sulfate, and the solvent was then distilled away under reduced pressure. The obtained residue was purified by silica gel column chromatography (eluent: chloroform-methanol) to obtain 2-methyl-6-(morpholine-4-carbonyl)quinolin-5-yl trifluoromethanesulfonate (129 mg) in the form of a light yellow solid.

### Production Example 260

A mixture of 2-methyl-6-(morpholine-4-carbonyl)quinolin-5-yl trifluoromethanesulfonate (Production Example 257; 59 mg), naphthalen-1-ylboronic acid (38 mg), THF (1.2 ml), water (0.3 ml), sodium carbonate (46 mg), and tetrakis(triphenylphosphine)palladium(0) (17 mg) was stirred under a nitrogen atmosphere at 30° C. for 3 hours. Thereafter, the solvent was distilled away from the reaction mixture under reduced pressure, and the obtained residue was then purified by silica gel column chromatography (eluent: chloroform-methanol), so as to obtain (2-methyl-5-(naphthalen-1-yl)quinolin-6-yl)(morpholino)methanone (62 mg) in the form of a light brown oily substance.

### Production Example 262

A mixture of (4-chloro-2-methylquinolin-6-yl)(morpholino)methanone (Production Example 34; 97 mg), 1-methyl-4-(2-(4,4,5,5-tetramethyl-1,3,2-dioxaborolan-2-yl)phenyl)-1H-pyrazole (Angewandte Chemie, International Edition, 53(45), 12077-12080, 2014; 114 mg), 1,2-dimethoxyethane (1.9 ml), a 3 M sodium carbonate aqueous solution (0.334 ml), and a [1,1′-bis(diphenylphosphino)ferrocene]dichloropalladium(II)dichloromethane adduct (27 mg) was stirred under a nitrogen atmosphere at 80° C. for 7 hours. Thereafter, chloroform was added to the reaction mixture, and the organic layer was washed with a saturated saline and was then dried over anhydrous sodium sulfate. After that, the solvent was distilled away under reduced pressure. The obtained residue was successively purified by silica gel column chromatography (eluent: chloroform-methanol) and then, by gel permeation chromatography (eluent: chloroform), so as to obtain (2-methyl-4-(2-(1-methyl-1H-pyrazol-4-yl)phenyl)quinolin-6-yl)(morpholino)methanone (54 mg) in the form of a colorless oily substance.

### Production Example 263

Trifluoroacetic anhydride (0.422 ml) was added to a mixture of 2-methyl-3-(naphthalen-1-yl)quinoline-6-carboxamide (Production Example 210; 380 mg) and chloroform (20 ml) under ice cold. The obtained mixture was stirred under ice cold for 30 minutes, and then, at room temperature for 30 minutes. Thereafter, a saturated sodium carbonate aqueous solution was added to the reaction mixture, and a generated product was then extracted with ethyl acetate. After that, the solvent was distilled away from the organic layer under reduced pressure. The obtained residue was purified by silica gel column chromatography (eluent: hexane-ethyl acetate), so as to obtain 2-methyl-3-(naphthalen-1-yl)quinoline-6-carbonitrile (299 mg) in the form of a yellow solid.

### Production Example 264

A mixture of 2-methyl-3-(naphthalen-1-yl)quinoline-6-carbonitrile (Production Example 263; 299 mg), trimethylsilyl azide (0.199 ml), and tetrabutylammonium fluoride hydrate (160 mg) was stirred at 85° C. for 1 hour 30 minutes. Thereafter, trimethylsilyl azide (0.199 ml) and tetrabutylammonium fluoride (160 mg) were added to the reaction mixture, and the thus obtained mixture was then stirred at 85° C. for 17 hours. Subsequently, 1 M hydrochloric acid was added to the reaction mixture, and a generated product was then extracted with ethyl acetate. A solid precipitated from the organic layer was collected by filtration, and was then suspended in 1 M hydrochloric acid (30 ml), followed by stirring the suspension at room temperature for 30 minutes. Thereafter, the obtained solid was collected by filtration, and was then dried under reduced pressure, so as to obtain 2-methyl-3-(naphthalen-1-yl)-6-(1H-tetrazol-5-yl)quinoline (150 mg) in the form of a yellow solid.

### Production Example 265

2-(Chloromethoxy)ethyltrimethylsilane (0.086 ml) was added to a mixture of 2-methyl-3-(naphthalen-1-yl)-6-(2H-tetrazol-5-yl)quinoline (Production Example 264; 150 mg), DMF (5 ml), and triethylamine (0.136 ml) under ice cold, and the obtained mixture was then stirred at room temperature for 30 minutes. Thereafter, ethyl acetate was added to the reaction mixture, and the thus obtained mixture was then washed with water. After that, the solvent was distilled away from the organic layer under reduced pressure. The obtained residue was purified by silica gel column chromatography (eluent: hexane-ethyl acetate), so as to obtain 2-methyl-3-(naphthalen-1-yl)-6-(N-((2-(trimethylsilyl)ethoxy)methyl)tetrazol-5-yl)quinoline (168 mg) in the form of a yellow oily substance.

### Production Example 266

2-Picoline borane (94 mg) was added to a mixture of 2-methylquinoline-4-carbaldehyde (manufactured by Combi-Blocks; 150 mg), morpholine (0.075 ml), methanol (4.55 ml), and acetic acid (0.45 ml), and the obtained mixture was then stirred at room temperature for 2 days. Thereafter, 1 M hydrochloric acid (5 ml) was added to the reaction mixture, and the thus obtained mixture was then stirred at room temperature for 30 minutes. Subsequently, the pH of the reaction mixture was adjusted to about pH 9 with a saturated sodium carbonate aqueous solution. A generated product was extracted with ethyl acetate, and the solvent was then distilled away from the organic layer under reduced pressure. The obtained residue was successively purified by silica gel column chromatography (eluent: hexane-ethyl acetate) and then, by aminopropyl silica gel column chromatography (eluent: hexane-ethyl acetate), so as to obtain 4-((2-methylquinolin-4-yl)methyl)morpholine (171 mg) in the form of a colorless oily substance.

### Production Example 267

A mixture of 2-methylquinoline-4-carbaldehyde (manufactured by Combi-Blocks; 150 mg), methyl 2-(2-aminoethoxy)acetate hydrochloride (manufactured by BLD Pharmatech; 236 mg), sodium triacetoxyborohydride (394 mg), triethylamine (0.324 ml), and trifluoroethanol (10 ml) was stirred at room temperature for 4 days. Thereafter, the solvent was distilled away from the reaction mixture under reduced pressure, and 1 M hydrochloric acid (5 ml) was then added to the obtained residue, followed by stirring the obtained mixture at room temperature for 30 minutes. Thereafter, a saturated sodium carbonate aqueous solution was added to the reaction mixture, and a generated product was then extracted with ethyl acetate. After that, the solvent was distilled away from the organic layer under reduced pressure. The obtained residue was purified by silica gel column chromatography (eluent: ethyl acetate-methanol), and was then washed with diisopropyl ether, so as to obtain 4-((2-methylquinolin-4-yl)methyl)morpholin-3-one (78 mg) in the form of a colorless solid.

### Production Example 269

A mixture of tert-butyl ((4-chloro-2-methylquinolin-6-yl)methyl(tetrahydro-2H-pyran-4-yl)carbamate (Production Example 420; 100 mg), 1-(6-(4,4,5,5-tetramethyl-1,3,2-dioxaborolan-2-yl)-3,4-dihydroisoquinolin-2(1H)-yl)ethan-1-one (manufactured by Enamine; 116 mg), toluene (0.77 ml), tripotassium phosphate (109 mg), 2-dicyclohexylphosphino-2′,6′-dimethoxybiphenyl (26 mg), and palladium acetate (II) (5.7 mg) was stirred under a nitrogen atmosphere at 100° C. for 3 hours. Thereafter, the reaction mixture was purified by silica gel column chromatography (eluent: chloroform-methanol), so as to obtain tert-butyl ((4-(2-acetyl-1,2,3,4-tetrahydroisoquinolin-6-yl)-2-methylquinolin-6-yl)methyl)(tetrahydro-2H-pyran-4-yl)carbamate (68 mg) in the form of a slight brown oily substance.

### Production Example 440

While the internal temperature was kept at −65° C. or lower, diisobutylaluminum hydride (1 M toluene solution, 8.4 ml) was added dropwise to a mixture of ethyl 4-chloro-2-methylquinoline-6-carboxylate (Production Example 66; 349 mg) and THF (17.5 ml), and the obtained mixture was then stirred at −78° C. for 2 hours. Thereafter, the reaction mixture was poured into ice water (100 ml), and a generated product was then extracted with ethyl acetate. After that, the extract was dried over anhydrous sodium sulfate, and the solvent was then distilled away under reduced pressure. The obtained residue was purified by silica gel column chromatography (eluent: chloroform-methanol), so as to obtain (4-chloro-2-methylquinolin-6-yl)methanol (217 mg) in the form of a slight yellow solid.

In Production Examples 11 to 13, 15, 17, 19 to 29, 32, 35 to 44, 46, 49, 52, 53, 55, 63, 69, 80, 82, 86, 88, 91, 92, 99 to 106, 110, 113 to 118, 120 to 180, 183, 184, 186, 187, 189, 191, 193, 195 to 198, 200 to 204, 207, 208, 210, 213, 215 to 219, 221, 222, 225 to 236, 241 to 247, 249 to 251, 254, 258, 259, 261, 268, 270 to 439, and 441, the compounds were synthesized according to the above-described methods or methods equivalent thereto. The compound names, structural formulae, synthetic method examples, raw material compounds, and physical property data (1H NMR chemical shift values and MS molecular ion peaks) of the compounds of individual production examples will be shown in the following table.

The solvent used in the measurement of 1H NMR is deuterated chloroform, unless otherwise specified.

TABLE 2

| Production | | | Synthetic | Raw | |
|---|---|---|---|---|---|
| Ex. | | | method | material | |
| No. | Compound name | Structural formula | Ex. | compound | Physical property data |
| 1 | N-acetyl-N-((1- acetyl-3- oxoindolin-4- yl)methyl) acetamide | 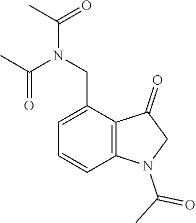 [SMILES: CC(=O)N(Cc1cccc2c1C(=O)CN2C(C)=O)C(C)=O] | | CAS No. 65923-35-5 (Reagent Supplier 1) | MS 289.36 (M + H). |
| 2 | N-((1-acetyl-3- oxoindolin-4- yl)methyl) acetamide | 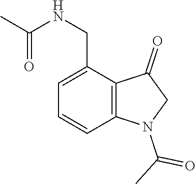 [SMILES: CC(=O)NCc1cccc2c1C(=O)CN2C(C)=O] | | Production Ex. 1 | MS 247.19 (M + H). |
| 3 | (E)-3-(4-formyl-2- methoxyphenyl)acr ylamide | 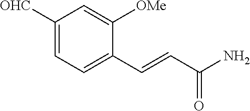 [SMILES: COc1cc(C=O)ccc1/C=C/C(N)=O] | | CAS No. 43192-34-3 (Reagent Supplier 2) | 1H NMR (270 MHz) δ 9.97 (s, 1H), 7.89 (d, J = 16.0 Hz, 1H), 7.63 (d, J = 7.7 Hz, 1H), 7.49-7.37 (m, 2H), 6.67 (d, J = 15.9 Hz, 1H), 5.58 (br s, 2H), 3.95 (s, 3H). MS 206.12 |
| | | | | | (M + H). |
| 4 | (E)-4-((2-(2- methylbenzo [d]thiazol-6- yl)vinyl) sulfonyl) morpholine | 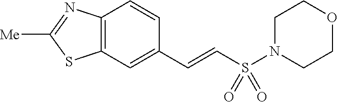 [SMILES: Cc1nc2ccc(/C=C/S(=O)(=O)N3CCOCC3)cc2s1] | | CAS No. 41067-80-5 (Synthetic Literature 1) CAS No. 5304- | 1H NMR (270 MHz) δ 8.02-7.92 (m, 2H), 7.67- 7.51 (m, 2H), 6.80-6.66 (m, 1H), 3.88-3.69 (m, 4H), 3.34-3.17 (m, 4H), 2.87 (s, 3H). MS 325.21 |
| | | | | 21-2 | (M + H). |
| | | | | (Reagent | |
| | | | | Supplier 3) | |
| 5 | (E)-2-(4-formyl- 2-methoxyphenyl) ethene-1- sulfonamide | 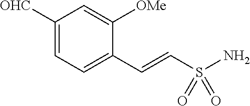 [SMILES: COc1cc(C=O)ccc1/C=C/S(N)(=O)=O] | | CAS No. 43192-34-3 (Reagent Supplier 2) | MS 242.11 (M + H). |
| 6 | 3-(4-formyl-2- methoxyphenyl) propanamide | 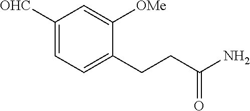 [SMILES: COc1cc(C=O)ccc1CCC(N)=O] | | Production Ex. 3 | 1H NMR (270 MHz) δ 9.95 (s, 1H), 7.46-7.31 (m, 3H), 5.36 (br s, 2H), 3.91 (s, 3H), 3.10-2.97 (m, 2H), 2.55 (dd, J = 8.3, 7.1 Hz, 2H). MS 208.11 (M + H). |
| 7 | 3-(2- methylquinolin-6- yl)-1- morpholinopropan- 1-one | 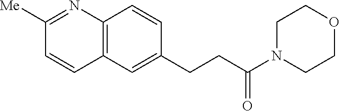 [SMILES: Cc1ccc2cc(CCC(=O)N3CCOCC3)ccc2n1] | | Production Ex. 18 | 1H NMR (270 MHz) δ 7.97 (t, J = 8.6 Hz, 2H), 7.63-7.50 (m, 2H), 7.27 (d, J = 8.4 Hz, 1H), 3.63 (s, 4H), 3.51 (dd, J = 5.8, 3.8 Hz, 2H), 3.43-3.33 (m, 2H), 3.22-3.10 (m, 2H), |
| | | | | | 2.77-2.65 (m, 5H). MS |
| | | | | | 285.28 (M + H). |
| 8 | 3-methoxy-4- ((methylsulfonyl) methoxy) benzaldehyde | 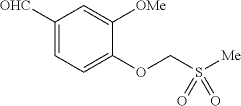 [SMILES: COc1cc(C=O)ccc1OCS(C)(=O)=O] | | CAS No. 18738-41-5 (Synthetic Literature 2) | 1H NMR (270 MHz) δ 9.92 (s, 1H), 7.52-7.44 (m, 2H), 7.31-7.25 (m, 1H), 5.09 (s, 2H), 3.96 (s, 3H), 3.08 (s, 3H). MS 245.03 (M + H). |
| 9 | 3-methoxy-4- ((methylsulfinyl) methoxy) benzaldehyde | 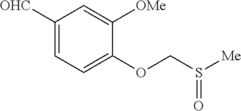 [SMILES: COc1cc(C=O)ccc1OCS(C)=O] | | CAS No. 18738-41-5 (Synthetic Literature 2) | 1H NMR (270 MHz) δ 9.91 (s, 1H), 7.52-7.42 (m, 2H), 7.35-7.23 (m, 1H), 5.08 (dd, J = 10.5, 10.4 Hz, 2H), 3.95 (s, 3H), 2.75 (s, 3H). MS 229.12 (M + H). |
| 10 | 2-(4-formyl-2- isopropoxyphenoxy) acetamide | 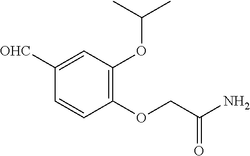 [SMILES: CC(C)Oc1cc(C=O)ccc1OCC(N)=O] | | CAS No. 71118-98- 4 (Synthetic Literature 3) | 1H NMR (270 MHz) δ 9.88 (s, 1H), 7.49-7.39 (m, 2H), 7.06-6.96 (m, 1H), 4.76-4.62 (m, 1H), 4.59 (s, 2H), 1.41 (d, J = 6.0 Hz, 6H). MS 238.20 (M + H). |
| 11 | 2-(4-formyl-3,5- dimethoxyphenoxy) acetamide | 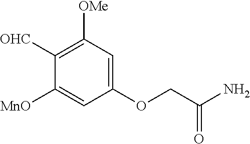 | Production Ex. 10 | CAS No. 22080-96-2 (Reagent Supplier 4) | 1H NMR (270 MHz) δ 10.37 (s, 1H), 6.12 (s, 2H), 4.58 (s, 2H), 3.90 (s, 6H). MS 240.24 (M + H). |
| 12 | 3-methoxy-4-(2- ((tetrahydro-2H- pyran-2- yl)oxy)ethoxy) benzaldehyde | 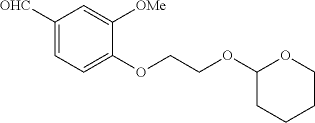 [SMILES: COc1cc(C=O)ccc1OCCOC1CCCCO1] | Production Ex. 10 | CAS No. 17739-45-6 (Reagent Supplier 4) | 1H NMR (270 MHz) δ 9.86 (s, 1H), 7.50-7.38 (m, 2H), 7.06 (d, J = 8.2 Hz, 1H), 4.75-4.68 (m, 1H), 4.42-4.23 (m, 2H), 4.20-4.05 (m, 1H), 4.01- 3.82 (m, 5H), 3.60-3.46 (m, 1H), 1.90-1.54 (m, |
| | | | | | 6H). MS 303.24 (M + Na). |
| 13 | 2-methyl-6-(2- ((tetrahydro-2H- pyran-2- yl)oxy)ethoxy) quinoline | 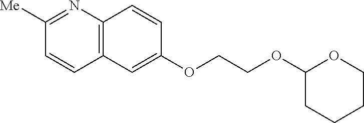 [SMILES: Cc1ccc2cc(OCCOC3CCCCO3)ccc2n1] | Production Ex. 10 | CAS No. 17739-45- 6 CAS No. 613-21- 8 (Reagent Supplier 4 for each of them) | 1H NMR (270 MHz) δ 7.93 (dd, J = 8.6, 4.6 Hz, 2H), 7.38 (dd, J = 9.2, 2.9 Hz, 1H), 7.24 (d, J = 8.3 Hz, 1H), 7.08 (d, J = 2.8 Hz, 1H), 4.79-4.70 (m, 1H), 4.32-4.22 (m, 2H), 4.13 (dt, J = 11.2, 4.5 Hz, |
| | | | | | 1H), 3.99-3.82 (m, 2H), |
| | | | | | 3.55 (d, J = 11.4 Hz, 1H), |
| | | | | | 2.71 (s, 3H), 1.88-1.51 |
| | | | | | (m, 6H). MS 288.36 |
| | | | | | (M + H). |
| 14 | 4-((2- methylquinolin-6- yl)methyl)thiomor pholine 1,1-dioxide | 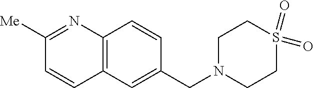 [SMILES: Cc1ccc2cc(CN3CCS(=O)(=O)CC3)ccc2n1] | | CAS No. 1393580- 26-1 (Reagent Supplier 5) | 1H NMR (270 MHz) δ 8.08-7.95 (m, 2H), 7.71- 7.63 (m, 2H), 7.31 (d, J = 8.4 Hz, 1H), 3.81 (s, 2H), 3.14-2.98 (m, 8H), 2.75 (s, 3H)). MS 291.19 |
| | | | | | (M + H). |
| 15 | 4-((2- methylquinolin-6- yl)methyl) morpholine | 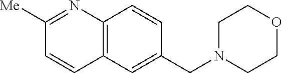 [SMILES: Cc1ccc2cc(CN3CCOCC3)ccc2n1] | Production Ex. 14 | CAS No. 1393580- 26-1 (Reagent Supplier 5) | 1H NMR (270 MHz) δ 8.05-7.95 (m, 2H), 7.73- 7.67 (m, 2H), 7.32-7.25 (m, 1H), 3.78-3.69 (m, 4H), 3.66 (s, 2H), 2.74 (s, |
| | | | | | 3H), 2.54-2.44 (m, |
| | | | | | 4H). MS 243.35 (M + H). |
| 16 | N-(4-formyl-2- methoxyphenyl)-2- morpholino- acetamide | 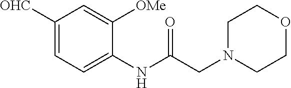 [SMILES: COc1cc(C=O)ccc1NC(=O)CN1CCOCC1] | | CAS No. 89531-58-8 (Reagent Supplier 4) CAS No. 90151- | 1H NMR (270 MHz, dimethylsulfoxide-d6) δ 10.10 (br s, 1H), 9.90 (s, 1H), 8.60 (d, J = 8.2 Hz, 1H), 7.50 (dd, J = 8.2, 1.7 Hz, 1H), 7.44 (d, J = 1.7 |
| | | | | 40-9 | Hz, 1H), 4.00 (s, 3H), 3.88- |
| | | | | (Reagent | 3.75 (m, 4H), 3.20 (s, |
| | | | | Supplier 6) | 2H), 2.71-2.60 (m, |
| | | | | | 4H). MS 279.21 (M + H). |
| 17 | (4-azido-2- methylquinolin-6- yl)(morpholino) methanone | 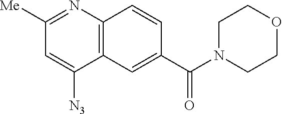 [SMILES: Cc1cc(N=[N+]=[N-])c2cc(C(=O)N3CCOCC3)ccc2n1] | Production Ex. 16 | Production Ex. 90 | 1H NMR (270 MHz) δ 8.10 (d, J = 1.9 Hz, 1H), 8.04 (d, J = 8.7 Hz, 1H), 7.73 (dd, J = 8.6, 1.9 Hz, 1H), 7.09 (s, 1H), 3.98- 3.41 (m, 8H), 2.77 (s, 3H). MS 298.26 (M + H). |
| 18 | (E)-3-(2- methylquinolin-6- yl)-1- morpholinoprop- 2-en-1-one | 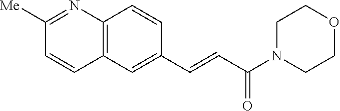 [SMILES: Cc1ccc2cc(/C=C/C(=O)N3CCOCC3)ccc2n1] | | CAS No. 1403962- 40-2 (Synthetic Literature 4) | 1H NMR (500 MHz) δ 8.06 (d, J = 8.4 Hz, 1H), 8.03-7.97 (m, 1H), 7.92- 7.83 (m, 3H), 7.32 (d, J = 8.4 Hz, 1H), 6.97 (d, J = 15.4 Hz, 1H), 3.83-3.66 (m, 8H), 2.76 (s, 3H). MS |
| | | | | | 283.18 (M + H). |
| 19 | (2-methylquinolin- 6- yl)(morpholino) methanone | 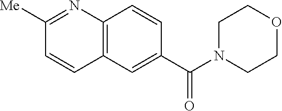 [SMILES: Cc1ccc2cc(C(=O)N3CCOCC3)ccc2n1] | Production Ex. 18 | CAS No. 635-80-3 (Reagent Supplier 4) | 1H NMR (500 MHz) δ 8.11-8.02 (m, 2H), 7.88 (d, J = 1.9 Hz, 1H), 7.68 (dd, J = 8.6, 1.9 Hz, 1H), 7.35 (d, J = 8.4 Hz, 1H), 4.01-3.36 (m, 8H), 2.77 (s, 3H). MS 257.16 |
| | | | | | (M + H). |
| 20 | (5-methylfuro[3,2- b]pyridin-2- yl)(morpholino) methanone | 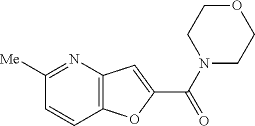 [SMILES: Cc1ccc2oc(C(=O)N3CCOCC3)cc2n1] | Production Ex. 18 | CAS No. 6497-45-8 (Reagent Supplier 2) | 1H NMR (270 MHz) δ 7.71 (dd, J = 8.6, 1.0 Hz, 1H), 7.38 (d, J = 1.0 Hz, 1H), 7.20 (d, J = 8.6 Hz, 1H), 3.91-3.72 (m, 8H), 2.68 (s, 3H). MS 247.23 (M + H). |
| 21 | tert-butyl 4-(2- methylquinoline- 6-carbonyl) piperazine-1- carboxylate | 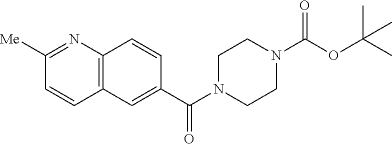 [SMILES: Cc1ccc2cc(C(=O)N3CCN(C(=O)OC(C)(C)C)CC3)ccc2n1] | Production Ex. 18 | CAS No. 635-80-3 (Reagent Supplier 4) CAS No. 57260- 71-6 (Reagent Supplier 7) | 1H NMR (270 MHz) δ 8.13-8.01 (m, 2H), 7.87 (d, J = 1.8 Hz, 1H), 7.68 (dd, J = 8.7, 1.9 Hz, 1H), 7.36 (d, J = 8.4 Hz, 1H), 3.87-3.29 (m, 8H), 2.77 (s, 3H), 1.47 (s, 9H). MS 356.35 (M + H). |
| 22 | tert-butyl 4-(4- ([1,1′-biphenyl]-2- yl)-2- methylquinoline-6- carbonyl)piperazine- 1-carboxylate | 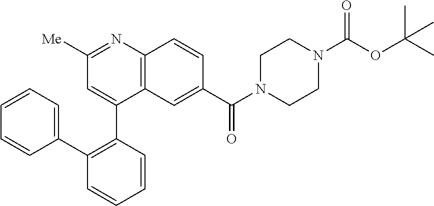 [SMILES: Cc1cc(-c2ccccc2-c2ccccc2)c2cc(C(=O)N3CCN(C(=O)OC(C)(C)C)CC3)ccc2n1] | Production Ex. 18 | Production Ex. 94 CAS No. 57260- 71- 6 (Reagent Supplier 7) | 1H NMR (500 MHz) δ 7.98 (d, J = 8.6 Hz, 1H), 7.60 (d, J = 1.8 Hz, 1H), 7.58-7.52 (m, 3H), 7.50- 7.44 (m, 1H), 7.37-7.32 (m, 1H), 7.12 (s, 1H), 7.08- 6.99 (m, 5H), 3.86-3.09 (m, 8H), 2.66 (s, 3H), 1.47 (s, 9H). MS 508.38 (M + H). |
| 23 | (E)-3-(2- methylbenzo[d] thiazol-5-yl)-1- morpholinoprop-2- en-1-one | 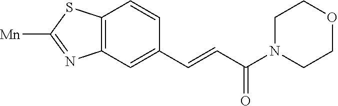 | Production Ex. 18 | CAS No. 20077-85-4 (Reagent Supplier 1) | 1H NMR (270 MHz) δ 8.09 (d, J = 1.7 Hz, 1H), 7.88-7.76 (m, 2H), 7.52 (dd, J = 8.4, 1.7 Hz, 1H), 6.93 (d, J = 15.3 Hz, 1H), 3.83-3.62 (m, 8H), 2.85 (s, 3H). MS 289.27 |
| | | | | | (M + H). |
| 24 | (2,6- dimethylmorpholino) (2-methylbenzo[d] thiazol-5- yl)methanone | 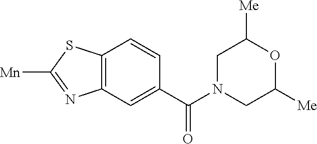 | Production Ex. 18 | CAS No. 24851-69-2 (Reagent Supplier 2) CAS No. 141-91- 3 (Reagent Supplier 8) | MS 291.33 (M + H). |
| 25 | (2,6- dimethylmorpholino) (2-methylbenzo[d] thiazol-6- yl)methanone | 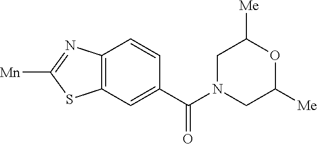 | Production Ex. 18 | CAS No. 6941-28-2 (Reagent Supplier 2) CAS No. 141-91- 3 (Reagent Supplier 8) | MS 291.34 (M + H). |
| 26 | (2,6- dimethylmorpholino) (2-methylquinolin-6- yl)methanone | 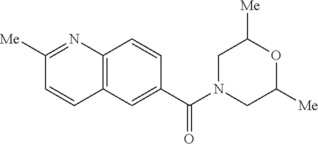 [SMILES: Cc1ccc2cc(C(=O)N3CC(C)OC(C)C3)ccc2n1] | Production Ex. 18 | CAS No. 635-80- 3 (Reagent Supplier 4) CAS No. 141-91- 3 (Reagent Supplier 8) | MS 285.34 (M + H). |
| 27 | (E)-3-(2- methylbenzo[d] thiazol-6-yl)-1- morpholinoprop-2- en-1-one | 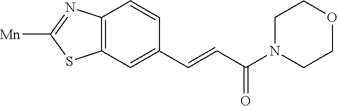 | Production Ex. 18 | CAS No. 3027-92-7 (Reagent Supplier 1) | 1H NMR (270 MHz) δ 7.96 (d, J = 1.7 Hz, 1H), 7.93 (d, J = 8.5 Hz, 1H), 7.80 (d, J = 15.5 Hz, 1H), 7.64 (dd, J = 8.5, 1.7 Hz, 1H), 6.90 (d, J = 15.5 Hz, 1H), 3.75 (s, 8H), 2.86 (s, |
| | | | | | 3H). MS 289.17 (M + H). |
| 28 | (E)-3-(2- methylquinolin-6- yl)-1-(4-(oxetan-3- yl)piperazin-1- yl)prop-2-en-1-one | 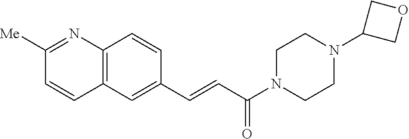 [SMILES: Cc1ccc2cc(/C=C/C(=O)N3CCN(C4COC4)CC3)ccc2n1] | Production Ex. 18 | CAS No. 1403962- 40-2 (Synthetic Literature 4) CAS No. 1404373- 75- 6 (Reagent | 1H NMR (500 MHz) δ 8.08-7.97 (m, 2H), 7.92- 7.79 (m, 3H), 7.31 (d, J = 8.4 Hz, 1H), 6.99 (d, J = 15.4 Hz, 1H), 4.69 (t, J = 6.5 Hz, 2H), 4.64 (t, J = 6.1 Hz, 2H), 3.90-3.69 (m, 4H), 3.57-3.48 (m, 1H), 2.75 (s, 3H), 2.38 (s, |
| | | | | Supplier 9) | 4H). MS 338.29 |
| | | | | | (M + H). |
| 29 | (E)-3-(2- methylquinolin-6- yl)-N-(1-(oxetan-3- yl)piperidin-4- yl)acrylamide | 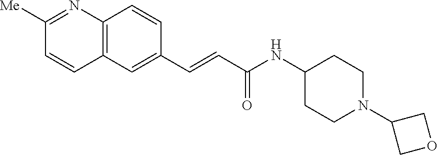 [SMILES: Cc1ccc2cc(/C=C/C(=O)NC3CCN(C4COC4)CC3)ccc2n1] | Production Ex. 18 | CAS No. 1403962- 40-2 (Synthetic Literature 4) CAS No. 2344680-31- 3 (Reagent Supplier 9) | 1H NMR (500 MHz) δ 8.07-7.95 (m, 2H), 7.88- 7.82 (m, 2H), 7.78 (d, J = 15.5 Hz, 1H), 7.32 (d, J = 8.4 Hz, 1H), 6.49 (d, J = 15.5 Hz, 1H), 5.54 (d, J = 8.2 Hz, 1H), 4.68 (t, J = 6.6 Hz, 2H), 4.61 (t, J = 6.2 Hz, 2H), 4.06-3.92 (m, 1H), 3.54-3.45 (m, 1H), |
| | | | | | 2.75 (s, 3H), 2.09-1.95 |
| | | | | | (m, 4H), 1.59-1.42 (m, |
| | | | | | 4H). MS 352.26 (M + H). |
| 30 | 4-([1,1′-biphenyl]- 2-yl)-2-methyl-N- (1-(oxetan-3- yl)piperidin-4- yl)quinoline-6- carboxamide | 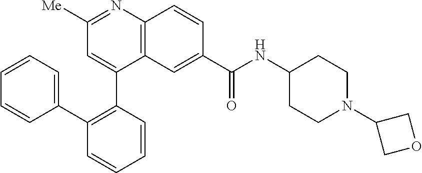 [SMILES: Cc1cc(-c2ccccc2-c2ccccc2)c2cc(C(=O)NC3CCN(C4COC4)CC3)ccc2n1] | | Production Ex. 94 CAS No. 2344680- 31- 3 (Reagent Supplier 9) | 1H NMR (500 MHz) δ 7.98-7.93 (m, 1H), 7.86- 7.80 (m, 2H), 7.60-7.53 (m, 2H), 7.52-7.46 (m, 1H), 7.43-7.37 (m, 1H), 7.20 (s, 1H), 7.08-6.99 (m, 5H), 5.90-5.80 (m, 1H), 4.65 (t, J = 6.6 Hz, 2H), 4.62-4.56 (m, 2H), 3.99-3.89 (m, 1H), 3.51- 3.42 (m, 1H), 2.78-2.70 |
| | | | | | (m, 2H), 2.69 (s, 3H), 2.10- |
| | | | | | 1.90 (m, 4H), 1.62-1.50 |
| | | | | | (m, 2H). MS 478.40 |
| | | | | | (M + H) |
| 31 | (4-amino-2- methylquinolin-6- yl)(morpholino) methanone | 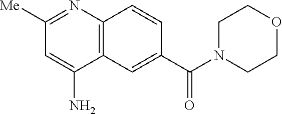 [SMILES: Cc1cc(N)c2cc(C(=O)N3CCOCC3)ccc2n1] | | Production Ex. 93 | 1H NMR (270 MHz) δ 7.98-7.88 (m, 2H), 7.59 (dd, J = 8.6, 1.9 Hz, 1H), 6.55 (s, 1H), 4.72 (br s, 2H), 3.90-3.47 (m, 8H), 2.61 (s, 3H). MS 272.18 (M + H). |
| 32 | 2-methyl-6- (morpholine-4- carbonyl)quinoline- 4-carboxamide | 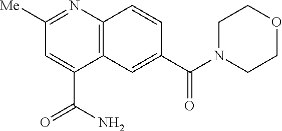 [SMILES: Cc1cc(C(N)=O)c2cc(C(=O)N3CCOCC3)ccc2n1] | Production Ex. 31 | Production Ex. 91 | 1H NMR (270 MHz, methanol-d4) δ 8.21 (d, J = 1.9 Hz, 1H), 7.98 (d, J = 8.6 Hz, 1H), 7.72 (dd, J = 8.7, 1.9 Hz, 1H), 7.52 (s, 1H), 3.84-3.32 (m, 8H), 2.68 (s, 3H). MS 300.19 (M + H). |
| 33 | (4-((3H- [1,2,3]triazolo[4,5- b]pyridin-3- yl)oxy)-2- methylquinolin-6- yl)(morpholino) methanone | 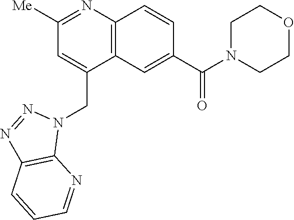 [SMILES: Cc1cc(Cn2nnc3cccnc32)c2cc(C(=O)N3CCOCC3)ccc2n1] | | Production Ex. 92 | 1H NMR (270 MHz, dimethylsulfoxide-d6) δ 8.88-8.79 (m, 2H), 8.43 (d, J = 1.8 Hz, 1H), 8.10 (d, J = 8.7 Hz, 1H), 7.91 (dd, J = 8.7, 1.8 Hz, 1H), 7.76- 7.67 (m, 1H), 6.80 (s, 1H), 3.66 (s, 8H), 2.52 (s, 3H). MS 391.30 (M + H). |
| 34 | (4-chloro-2- methylquinolin-6- yl)(morpholino) methanone | 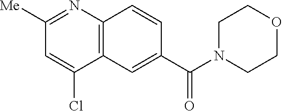 [SMILES: Cc1cc(Cl)c2cc(C(=O)N3CCOCC3)ccc2n1] | | Production Ex. 92 | 1H NMR (270 MHz) δ 8.27 (d, J = 1.8 Hz, 1H), 8.07 (d, J = 8.6 Hz, 1H), 7.75 (dd, J = 8.6, 1.8 Hz, 1H), 7.46 (s, 1H), 3.97- 3.39 (m, 8H), 2.75 (s, 3H). MS 291.2 (M + H). |
| 35 | tert-butyl 1-(2- methylquinoline-6- carbonyl)piperidine- 4-carboxylate | 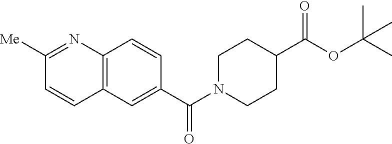 [SMILES: Cc1ccc2cc(C(=O)N3CCC(C(=O)OC(C)(C)C)CC3)ccc2n1] | Production Ex. 34 | CAS No. 635-80- 3 (Reagent Supplier 4) CAS No. 138007-24-6 (Reagent Supplier 9) | MS 355.39 (M + H). |
| 36 | (6- (hydroxymethyl) naphthalen-2- yl)(morpholino) methanone | 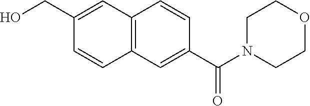 [SMILES: O=C(c1ccc2cc(CO)ccc2c1)N1CCOCC1] | Production Ex. 34 | CAS No. 5859-95-0 (Reagent Supplier 1) | 1H NMR (270 MHz) δ 7.93-7.80 (m, 4H), 7.58- 7.44 (m, 2H), 4.89 (d, J = 5.5 Hz, 2H), 3.95-3.44 (m, 8H), 1.97 (t, J = 6.0 Hz, 1H). MS 272.26 (M + H). |
| 37 | (2-methylquinolin- 6-yl)(4- morpholinopiperidin- 1-yl)methanone | 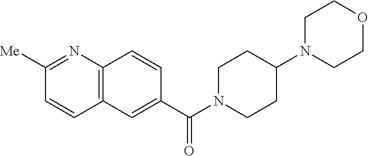 [SMILES: Cc1ccc2cc(C(=O)N3CCC(N4CCOCC4)CC3)ccc2n1] | Production Ex. 34 | CAS No. 635-80- 3 (Reagent Supplier 4) CAS No. 53617- 35-9 (Reagent Supplier 7) | 1H NMR (270 MHz) δ 8.12-7.99 (m, 2H), 7.86 (d, J = 1.6 Hz, 1H), 7.68 (dd, J = 8.6, 1.9 Hz, 1H), 7.35 (d, J = 8.4 Hz, 1H), 3.79-3.68 (m, 4H), 3.18- 2.82 (m, 2H), 2.77 (s, 3H), 2.62-2.39 (m, 5H), 2.15- 1.76 (m, 2H), 1.71-1.41 (m, 4H). MS 340.43 |
| | | | | | (M + H). |
| 38 | (4-(2- methoxyethyl) piperazin-1-yl)(2- methylquinolin-6- yl)methanone | 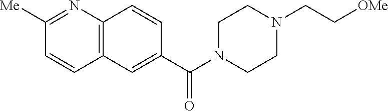 [SMILES: COCCN1CCN(C(=O)c2ccc3nc(C)ccc3c2)CC1] | Production Ex. 34 | CAS No. 635-80- 3 (Reagent Supplier 4) CAS No. 13484- 40- | 1H NMR (270 MHz) δ 8.13-7.99 (m, 2H), 7.87 (d, J = 1.9 Hz, 1H), 7.69 (dd, J = 8.6, 1.9 Hz, 1H), 7.34 (d, J = 8.5 Hz, 1H), 4.01-3.40 (m, 6H), 3.36 (s, 3H), 2.77 (s, 3H), 2.69- |
| | | | | 7 (Reagent | 2.38 (m, 6H). MS 314.38 |
| | | | | Supplier | (M + H). |
| | | | | 10) | |
| 39 | (2- methylbenzo[d] thiazo1-5- yl)(morpholino) methanone | 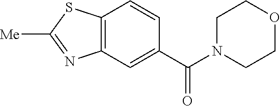 [SMILES: Cc1nc2cc(C(=O)N3CCOCC3)ccc2s1] | Production Ex. 34 | CAS No. 24851-69-2 (Reagent Supplier 2) | 1H NMR (270 MHz) δ 7.97 (d, J = 1.6 Hz, 1H), 7.89 (d, J = 8.2 Hz, 1H), 7.44 (dd, J = 8.2, 1.5 Hz, 1H), 3.91-3.41 (m, 8H), 2.87 (s, 3H). MS 263.22 (M + H). |
| 40 | (2- (hydroxymethyl)- 1H-indo1-5- yl)(morpholino) methanone | 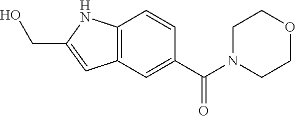 [SMILES: O=C(c1ccc2[nH]c(CO)cc2c1)N1CCOCC1] | Production Ex. 34 | CAS No. 1892727- 57-9 (Reagent Supplier 11) | 1H NMR (270 MHz) δ 9.12 (s, 1H), 7.53 (d, J = 1.4 Hz, 1H), 7.07 (dd, J = 8.4, 1.6 Hz, 1H), 6.98 (d, J = 8.3 Hz, 1H), 6.24 (d, J = 1.9 Hz, 1H), 4.70 (s, 2H), 3.97-3.45 (m, 8H). |
| | | | | | MS 261.00 (M + H). |
| 41 | (1,1- dioxidothiomorpho lino )(2- methylquinolin-6- yl)methanone | 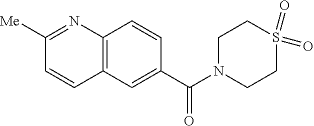 [SMILES: Cc1ccc2cc(C(=O)N3CCS(=O)(=O)CC3)ccc2n1] | Production Ex. 34 | CAS No. 635-80- 3 CAS No. 39093-93-1 (Reagent Supplier 4 for each of them) | 1H NMR (500 MHz, dimethylsulfoxide-d6) 8 8.34-8.29 (m, 1H), 8.10 (d, J = 1.9 Hz, 1H), 8.00- 7.95 (m, 1H), 7.79 (dd, J = 8.6, 1.9 Hz, 1H), 7.50 (d, J = 8.4 Hz, 1H), 4.16-3.62 (m, 4H), 3.39-3.18 (m, |
| | | | | | 4H), 2.68 (s, 3H). MS |
| | | | | | 305.30 (M + H). |
| 42 | 5-(morpholine-4- carbonyl)-1H- indole-3- carbaldehyde | 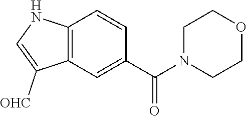 [SMILES: O=Cc1c[nH]c2ccc(C(=O)N3CCOCC3)cc12] | Production Ex. 34 | CAS No. 148563-41-1 (Reagent Supplier 2) | 1H NMR (500 MHz) δ 11.41 (s, 1H), 9.94 (s, 1H), 8.28 (d, J = 8.1 Hz, 1H), 7.70 (d, J = 3.0 Hz, 1H), 7.24 (dd, J = 8.1, 1.4 Hz, 1H), 7.12 (d, J = 1.4 Hz, 1H), 3.95-3.36 (m, 8H). MS 259.18 (M + H). |
| 43 | 1-methyl-5- (morpholine-4- carbonyl)-1H- indole-3- carbaldehyde | 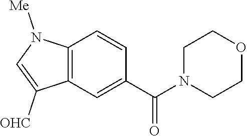 [SMILES: Cn1cc(C=O)c2cc(C(=O)N3CCOCC3)ccc21] | Production Ex. 34 | CAS No. 1557698- 86-8 (Reagent Supplier 5) | 1H NMR (500 MHz) δ 10.02 (s, 1H), 8.32 (d, J = 8.2 Hz, 1H), 7.77 (s, 1H), 7.57 (d, J = 1.4 Hz, 1H), 7.32 (dd, J = 8.1, 1.4 Hz, 1H), 3.91 (s, 3H), 3.89- 3.44 (m, 8H). MS 273.19 (M + H). |
| 44 | (5-methyl-1H- pyrrolo [3,2- b]pyridin-2- yl)(morpholino) methanone | 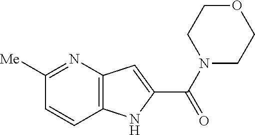 [SMILES: Cc1ccc2[nH]c(C(=O)N3CCOCC3)cc2n1] | Production Ex. 34 | CAS No. 1242427- 34- 4 (Reagent Supplier 1) | MS 246.20 (M + H). |
| 45 | N-((2- methylquinolin-6- yl)methyl)-2- morpholinoacetamide | 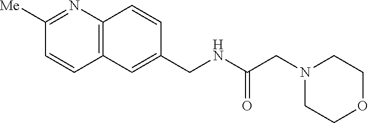 [SMILES: Cc1ccc2cc(CNC(=O)CN3CCOCC3)ccc2n1] | | CAS No. 89531-58-8 (Reagent Supplier 4) CAS No. 1369207- 07- | 1H NMR (270 MHz) δ 8.07-7.96 (m, 2H), 7.67 (d, J = 1.9 Hz, 1H), 7.60 (dd, J = 8.7, 2.1 Hz, 1H), 7.31 (d, J = 8.4 Hz, 1H), 4.66 (d, J = 6.3 Hz, 2H), 3.73-3.63 (m, 4H), 3.11 (s, 2H), 2.75 (s, 3H), 2.60- |
| | | | | 7 (Reagent | 2.50 (m, 4H). MS 300.28 |
| | | | | Supplier 9) | (M + H). |
| 46 | (2-methylquinolin- 6-yl)(4- phenylpiperidin-1- yl)methanone | 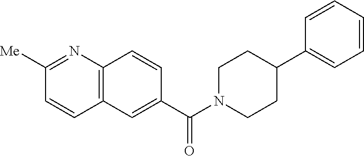 [SMILES: Cc1ccc2cc(C(=O)N3CCC(c4ccccc4)CC3)ccc2n1] | Production Ex. 45 | CAS No. 635-80- 3 (Reagent Supplier 4) Cas No. 10272-49-8 (Reagent Supplier 6) | 1H NMR (270 MHz) δ 8.14-8.01 (m, 2H), 7.90 (d, J = 1.7 Hz, 1H), 7.73 (dd, J = 8.6, 1.9 Hz, 1H), 7.39-7.29 (m, 3H), 7.26- 7.19 (m, 3H), 5.09-4.80 (m, 2H), 4.10-3.76 (m, 2H), 2.90-2.80 (m, 1H), 2.77 (s, 3H), 2.09-1.62 (m, 4H). MS 331.39 |
| | | | | | (M + H). |
| 47 | (2- (hydroxymethyl)- 1-methyl-1H- indol-5- yl)(morpholino) methanone | 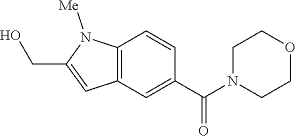 [SMILES: Cn1c(CO)cc2cc(C(=O)N3CCOCC3)ccc21] | | CAS No. 2091358- 20-0 (Reagent Supplier 11) | 1H NMR (270 MHz) δ 7.63-7.55 (m, 1H), 7.28- 7.24 (m, 2H), 6.38 (s, 1H), 4.76 (s, 2H), 3.87-3.51 (m, 11H), 2.69 (br s, 1H). MS 275.21 (M + H). |
| 48 | N-((2- methylquinolin-6- yl)methyl)acetamide | 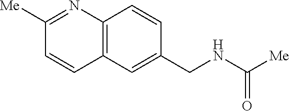 [SMILES: CC(=O)NCc1ccc2nc(C)ccc2c1] | | CAS No. 1369207- 07- 7 (Reagent Supplier 9) | 1H NMR (270 MHz) δ 8.06-7.94 (m, 2H), 7.70- 7.63 (m, 1H), 7.60 (dd, J = 8.6, 2.0 Hz, 1H), 7.29 (d, J = 8.4 Hz, 1H), 5.85 (s, 1H), 4.61 (d, J = 5.9 Hz, 2H), 2.74 (s, 3H), 2.07 (s, |
| | | | | | 3H). MS 215.26 (M + H). |
| 49 | N-((2- methylquinolin-6- yl)methyl)-N- (tetrahydro-2H- pyran-4- yl)acetamide | 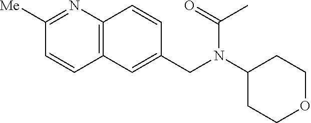 [SMILES: CC(=O)N(Cc1ccc2nc(C)ccc2c1)C1CCOCC1] | Production Ex. 48 | CAS No. 1981263- 96-0 (Reagent Supplier 1) | 1H NMR (270 MHz) 8 8.07-7.90 (m, 2H), 7.61- 7.47 (m, 2H), 7.36-7.23 (m, 1H), 4.98-4.62 (m, 3H), 4.03-3.85 (m, 2H), 3.55-3.30 (m, 2H), 2.78- 2.66 (m, 3H), 2.36-2.07 (m, 3H), 1.94-1.51 (m, |
| | | | | | 4H). MS 299.32 (M + H). |
| 50 | N-(2-methyl-6- (morpholine-4- carbonyl)quinolin- 4-yl)acetamide | 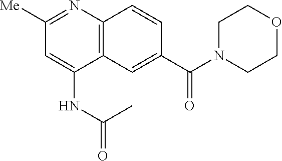 [SMILES: CC(=O)Nc1cc(C)nc2ccc(C(=O)N3CCOCC3)cc12] | | Production Ex. 31 | 1H NMR (270 MHz) δ 9.14 (br s, 1H), 8.13-8.02 (m, 2H), 7.82 (d, J = 8.6 Hz, 1H), 7.48 (dd, J = 8.7, 1.8 Hz, 1H), 3.96-3.34 (m, 8H), 2.68 (s, 3H), 2.31 (s, 3H). MS 314.20 (M + H). |
| 51 | 6-(2-morpholino-2- oxoethoxy)-[1,1′- biphenyl]-3- carbaldehyde | 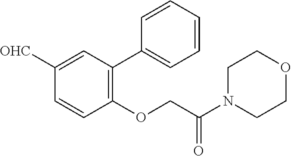 [SMILES: O=Cc1ccc(OCC(=O)N2CCOCC2)c(-c2ccccc2)c1] | | CAS No. 51068-78- 1 (Reagent Supplier 2) CAS No. 21363- 10-0 (Reagent Supplier 9) | MS 326.25 (M + H). |
| 52 | 6-fluoro-5-(2- morpholino-2- oxoethoxy) picolinaldehyde | 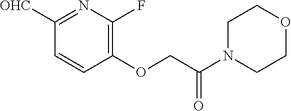 [SMILES: O=Cc1ccc(OCC(=O)N2CCOCC2)c(F)n1] | Production Ex. 51 | CAS No. 51068-78- 1 (Reagent Supplier 2) Cas No. 1211585-11- 3 (Reagent | MS 269.19 (M + H). |
| | | | | Supplier | |
| | | | | 12) | |
| 53 | 2-((2- methylquinolin-6- yl)oxy)-1- morpholinoethan- 1-one | 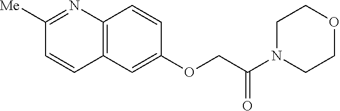 [SMILES: Cc1ccc2cc(OCC(=O)N3CCOCC3)ccc2n1] | Production Ex. 51 | CAS No. 51068-78-1 (Reagent Supplier 2) CAS No. 613-21-8 (Reagent | 1H NMR (270 MHz) δ 8.02-7.90 (m, 2H), 7.37 (dd, J = 9.2, 2.9 Hz, 1H), 7.27 (d, J = 8.4 Hz, 1H), 7.15 (d, J = 2.8 Hz, 1H), 4.82 (s, 2H), 3.72-3.60 (m, 8H), 2.71 (s, 3H). MS |
| | | | | Supplier 4) | 287.30 (M + H). |
| 54 | 2-fluoro-6- formylpyridin-3-yl methanesulfonate | 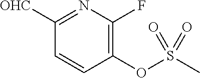 | | Cas No. 1211585-11- 3 (Reagent Supplier 12) | 1H NMR (270 MHz) δ 9.94 (s, 1H), 8.07-7.90 (m, 2H), 3.36 (s, 3H). MS 220.05 (M + H). |
| 55 | 6-formylpyridin-3- yl methanesulfonate | 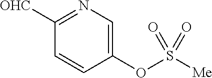 [SMILES: CS(=O)(=O)Oc1ccc(C=O)nc1] | Production Ex. 54 | CAS No. 31191-08-9 (Reagent Supplier 3) | 1H NMR (270 MHz) δ 10.12-10.05 (m, 1H), 8.73 (d, J = 2.5 Hz, 1H), 8.07 (d, J = 8.4 Hz, 1H), 7.90-7.79 (m, 1H), 3.30 (s, 3H). MS 202.19 (M + H). |
| 56 | methyl 1- (methylsulfonyl)- 1H-pyrrolo [3,2- b]pyridine-5- carboxylate | 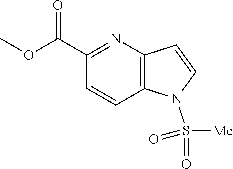 [SMILES: COC(=O)c1ccc2c(ccn2S(C)(=O)=O)n1] | | Cas No. 872355-63-0 (Synthetic Literature 5) | MS 255.11 (M + H). |
| 57 | 1- (methylsulfonyl)- 1H-pyrrolo [3,2- b]pyridine-5- carbaldehyde | 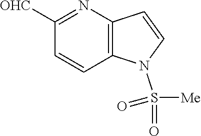 [SMILES: CS(=O)(=O)n1ccc2nc(C=O)ccc21] | | Production Ex. 56 | MS 225.11 (M + H). |
| 58 | (3-methoxyoxetan- 3-yl)methyl 4- methylbenzene- sulf onate | 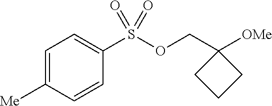 [SMILES: COC1(COS(=O)(=O)c2ccc(C)cc2)CCC1] | | CAS No. 1620017- 02-8 (Synthetic Literature 6) | MS 273.09 (M + H). |
| 59 | N-((2- methylquinolin-6- yl)methyl)methane sulfonamide | 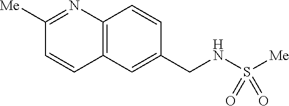 [SMILES: Cc1ccc2cc(CNS(C)(=O)=O)ccc2n1] | | CAS No. 1369207- 07- 7 (Reagent Supplier 9) | 1H NMR (270 MHz) δ 8.09-7.98 (m, 2H), 7.76 (d, J = 2.2 Hz, 1H), 7.66 (dd, J = 8.7, 2.1 Hz, 1H), 7.32 (d, J = 8.4 Hz, 1H), 4.76 (br s, 1H), 4.50 (d, J = 6.1 Hz, 2H), 2.90 (s, 3H), |
| | | | | | 2.75 (s, 3H). MS 251.25 |
| | | | | | (M + H). |
| 60 | N-((2- methylquinolin-6- yl)methyl)-N- (tetrahydro-2H- pyran-4-yl) methanesulfonamide | 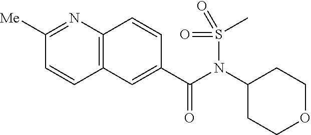 [SMILES: Cc1ccc2cc(C(=O)N(C3CCOCC3)S(C)(=O)=O)ccc2n1] | | CAS No. 1981263- 96-0 (Reagent Supplier 1) | 1H NMR (270 MHz) δ 8.09-7.96 (m, 2H), 7.78 (d, J = 2.0 Hz, 1H), 7.73 (dd, J = 8.6, 2.1 Hz, 1H), 7.31 (d, J = 8.3 Hz, 1H), 4.58 (s, 2H), 4.13-3.88 (m, 3H), 3.46-3.33 (m, 2H), 2.87 (s, 3H), 2.75 (s, |
| | | | | | 3H), 1.90-1.60 (m, |
| | | | | | 4H). MS 335.33 (M + H). |
| 61 | 5-methyl-2- (morpholine-4- carbonyl)-1H- pyrrolo [3,2- b]pyridine 4-oxide | 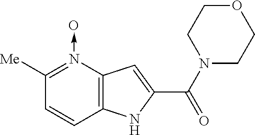 [SMILES: Cc1ccc2[nH]c(C(=O)N3CCOCC3)cc2[n+]1[O-]] | | Production Ex. 44 | 1H NMR (270 MHz) δ 9.76 (br s, 1H), 7.32 (d, J = 8.4 Hz, 1H), 7.23-7.19 (m, 1H), 7.14 (d, J = 8.5 Hz, 1H), 4.05-3.73 (m, 8H), 2.64 (s, 3H). MS 262.19 (M + H). |
| 62 | 5-methyl-2- (morpholine-4- carbonyl)furo[3,2- b]pyridine 4-oxide | 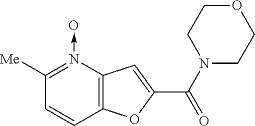 [SMILES: Cc1ccc2oc(C(=O)N3CCOCC3)cc2[n+]1[O-]] | | Production Ex. 20 | MS 263.26 (M + H). |
| 63 | 6- (ethoxycarbonyl)- 2-methylquinoline 1-oxide | 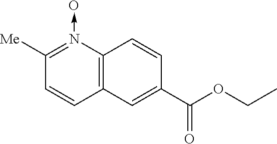 [SMILES: CCOC(=O)c1ccc2c(ccc(C)[n+]2[O-])c1] | Production Ex. 62 | CAS No. 855763-77- 8 (Reagent Supplier 3) | 1H NMR (270 MHz) δ 8.83 (d, J = 9.1 Hz, 1H), 8.60 (d, J = 1.9 Hz, 1H), 8.34 (dd, J = 9.2, 1.7 Hz, 1H), 7.76 (d, J = 8.6 Hz, 1H), 7.41 (d, J = 8.6 Hz, 1H), 4.47 (q, J = 7.1 Hz, 2H), 2.75 (s, 3H), 1.46 (t, J = 7.1 Hz, 3H). MS 232.16 |
| | | | | | (M + H). |
| 64 | (5- (hydroxymethyl)- 1H-pyrrolo [3,2- b]pyridin-2- yl)(morpholino) methanone | 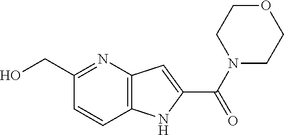 [SMILES: O=C(c1cc2nc(CO)ccc2[nH]1)N1CCOCC1] | | Production Ex. 61 | 1H NMR (270 MHz) δ 9.59 (br s, 1H), 7.80-7.70 (m, 1H), 7.17 (d, J = 8.5 Hz, 1H), 6.98-6.90 (m, 1H), 4.87 (s, 2H), 4.07- 3.88 (m, 4H), 3.86-3.75 (m, 4H). MS 262.26 (M + H). |
| 65 | (5- (hydroxymethyl)fu ro[3,2-b]pyridin-2- yl)(morpholino) methanone | 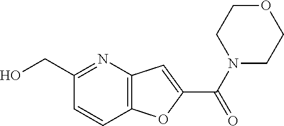 [SMILES: O=C(c1cc2nc(CO)ccc2o1)N1CCOCC1] | | Production Ex. 62 | 1H NMR (270 MHz) δ 7.83 (dd, J = 8.7, 1.1 Hz, 1H), 7.43 (d, J = 1.0 Hz, 1H), 7.31 (d, J = 8.6 Hz, 1H), 4.89 (d, J = 5.2 Hz, 2H), 3.91-3.75 (m, 8H), 3.53 (t, J = 5.2 Hz, 1H). MS 263.25 (M + H). |
| 66 | ethyl 4-chloro-2- methylquinoline-6- carboxylate | 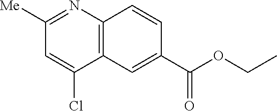 [SMILES: CCOC(=O)c1ccc2nc(C)cc(Cl)c2c1] | | Production Ex. 63 | 1H NMR (270 MHz) δ 8.93 (d, J = 1.9 Hz, 1H), 8.34 (dd, J = 8.8, 1.9 Hz, 1H), 8.06 (d, J = 8.9 Hz, 1H), 7.47 (s, 1H), 4.47 (q, J = 7.1 Hz, 2H), 2.76 (s, 3H), 1.46 (t, J = 7.1 Hz, |
| | | | | | 3H). MS 250.21 (M + H). |
| 67 | N-methyl-N-((2- methylquinolin-6- yl)methyl)tetrahydro- 2H-pyran-4- amine | 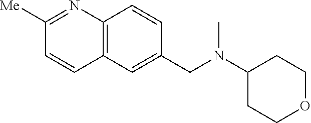 [SMILES: Cc1ccc2cc(CN(C)C3CCOCC3)ccc2n1] | | CAS No. 1981263- 96-0 (Reagent Supplier 1) | 1H NMR (270 MHz) δ 8.04-7.94 (m, 2H), 7.72- 7.64 (m, 2H), 7.28 (d, J = 8.3 Hz, 1H), 4.11-4.01 (m, 2H), 3.74 (s, 2H), 3.45- 3.32 (m, 2H), 2.79-2.61 (m, 4H), 2.24 (s, 3H), 1.87- 1.63 (m, 4H). MS 271.31 (M + H). |
| 68 | N-((2- methylbenzo[d] thiazol-6- yl)methyl) tetrahydro- 2H-pyran-4- amine | 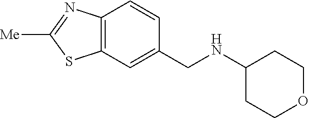 [SMILES: Cc1nc2ccc(CNC3CCOCC3)cc2s1] | | Cas No. 20061-51-2 (Reagent Supplier 5) CAS No. 38041-19- 9 (Reagent Supplier 13) | 1H NMR (270 MHz) δ 7.89 (d, J = 8.3 Hz, 1H), 7.81 (d, J = 1.6 Hz, 1H), 7.40 (dd, J = 8.3, 1.7 Hz, 1H), 4.03-3.91 (m, 4H), 3.46-3.34 (m, 2H), 2.83 (s, 3H), 2.82-2.67 (m, 1H), 1.93-1.82 (m, 2H), |
| | | | | | 1.50-1.38 (m, 2H). MS |
| | | | | | 263.33 (M + H). |
| 69 | N-((2- methylbenzo[d] thiazo1-5- yl)methyl) tetrahydro- 2H-pyran-4- amine | 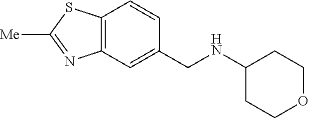 [SMILES: Cc1nc2cc(CNC3CCOCC3)ccc2s1] | Production Ex. 68 | Cas No. 20061-46-5 (Reagent Supplier 5) CAS No. 38041-19- 9 (Reagent Supplier13) | 1H NMR (270 MHz) δ 7.89 (d, J = 1.5 Hz, 1H), 7.78 (d, J = 8.2 Hz, 1H), 7.35 (dd, J = 8.2, 1.7 Hz, 1H), 4.05-3.91 (m, 4H), 3.44-3.32 (m, 2H), 2.84 (s, 3H), 2.82-2.67 (m, 1H), 1.93-1.82 (m, 2H), |
| | | | | | 1.54-1.37 (m, 2H). MS |
| | | | | | 263.18 (M + H). |
| 70 | N-((2- methylquinolin-6- yl)methyl)-1- (oxetan-3- yl)piperidin-4- amine | 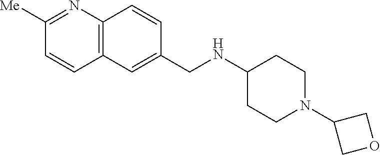 [SMILES: Cc1ccc2cc(CNC3CCN(C4COC4)CC3)ccc2n1] | | CAS No. 1228948- 05-7 (Synthetic Literature 7) CAS No. 108166-03-6 (Reagent Supplier 7) | 1H NMR (270 MHz) δ 8.04-7.95 (m, 2H), 7.70 (d, J = 1.9 Hz, 1H), 7.65 (dd, J = 8.6, 2.0 Hz, 1H), 7.27 (d, J = 8.3 Hz, 1H), 4.68-4.56 (m, 4H), 3.98 (s, 2H), 3.51-3.39 (m, 1H), 2.79-2.49 (m, 6H), 2.04-1.80 (m, 4H), 1.59- 1.39 (m, 2H). MS 312.40 |
| | | | | | (M + H). |
| 71 | (4-([1,l′-biphenyl]- 2-yl)-2- methylquinolin-6- yl)(piperazin-1- yl)methanone | 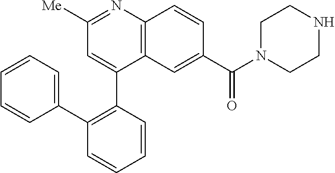 [SMILES: Cc1cc(-c2ccccc2-c2ccccc2)c2cc(C(=O)N3CCNCC3)ccc2n1] | | Production Ex. 22 | 1H NMR (500 MHz) δ 7.99 (d, J = 8.6 Hz, 1H), 7.61-7.52 (m, 4H), 7.50- 7.45 (m, 1H), 7.36-7.33 (m, 1H), 7.16 (s, 1H), 7.07- 7.00 (m, 5H), 4.14-2.86 (m, 8H), 2.67 (s, 3H). MS 408.33 (M + H) |
| 72 | (2-methylquinolin- 6-yl)(4-(oxetan-3- yl)piperazin-1- yl)methanone | 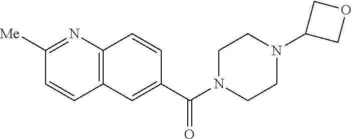 [SMILES: Cc1ccc2cc(C(=O)N3CCN(C4COC4)CC3)ccc2n1] | | CAS No. 1577354-59-6 (Reagent Supplier 1) CAS No. 6704-31-0 (Reagent Supplier 4) | 1H NMR (270 MHz) δ 8.11-8.01 (m, 2H), 7.87 (d, J = 1.8 Hz, 1H), 7.68 (dd, J = 8.6, 1.9 Hz, 1H), 7.35 (d, J = 8.3 Hz, 1H), 4.72-4.58 (m, 4H), 4.00- 3.34 (m, 5H), 2.77 (s, 3H), 2.49-2.22 (m, 4H). MS 312.35 (M + H). |
| 73 | 2-methyl-6-((4- (oxetan-3- yl)piperazin-1- yl)methyl)quinoline | 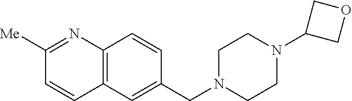 [SMILES: Cc1ccc2cc(CN3CCN(C4COC4)CC3)ccc2n1] | | CAS No. 1228948-05- 7 (Reagent Supplier 7) CAS No. 1404373-75- 6 (Reagent | 1H NMR (270 MHz) δ 8.07-7.99 (m, 2H), 7.71- 7.64 (m, 2H), 7.33-7.27 (m, 1H), 4.70-4.58 (m, 4H), 3.68 (s, 2H), 3.57- 3.45 (m, 1H), 2.74 (s, 3H), 2.63-2.32 (m, 6H), 1.71- |
| | | | | Supplier 9) | 1.58 (m, 2H). MS 298.40 |
| | | | | | (M + H). |
| 74 | (4-([1,1′-biphenyl]- 2-yl)-2- methylquinolin-6- yl)(4-(oxetan-3- yl)piperazin-1- yl)methanone | 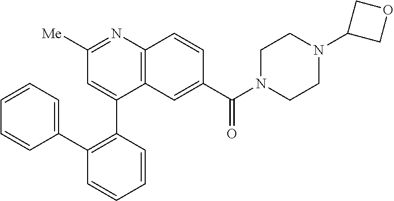 [SMILES: Cc1cc(-c2ccccc2-c2ccccc2)c2cc(C(=O)N3CCN(C4COC4)CC3)ccc2n1] | | Production Ex. 71 CAS No. 6704- 31-0 (Reagent Supplier 4) | 1H NMR (500 MHz) δ 7.97 (d, J = 8.6 Hz, 1H), 7.62 (d, J = 1.9 Hz, 1H), 7.58-7.52 (m, 3H), 7.49- 7.44 (m, 1H), 7.37-7.33 (m, 1H), 7.11 (s, 1H), 7.05- 7.01 (m, 5H), 4.69-4.64 (m, 2H), 4.60 (t, J = 6.2 Hz, 2H), 3.97-3.58 (m, 2H), 3.54-3.45 (m, 1H), 3.39-3.18 (m, 2H), 2.65 (s, 3H), 2.50-2.03 (m, 4H). MS 464.40 (M + H) |
| 75 | (2SR,6SR)-2,6- dimethyl-N-((2- methylquinolin-6- yl)methyl)tetrahydro- 2H-pyran-4- amine | 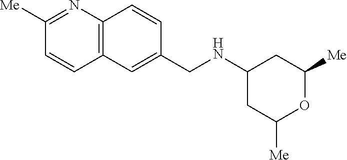 [SMILES: Cc1ccc2cc(CNC3CC(C)O[C@H](C)C3)ccc2n1] | | CAS No. 1073-79-6 (Reagent Supplier 3) Cas No. 1369207-07- 7 (Reagent Supplier 9) | 1H NMR (270 MHz) δ 8.07-7.93 (m, 2H), 7.74- 7.63 (m, 2H), 7.31-7.25 (m, 1H), 4.06-3.87 (m, 4H), 3.17-3.09 (m, 1H), 2.74 (s, 3H), 1.69-1.60 (m, 2H), 1.48-1.35 (m, 2H), 1.18 (s, 3H), 1.15 (s, 3H). MS 285.29 (M + H). |
| | | Racemate | | | |
| 76 | (2SR,6RS)-2,6- dimethyl-N-((2- methylquinolin-6- yl)methyl)tetrahydro- 2H-pyran-4- amine | 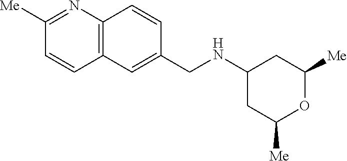 [SMILES: Cc1ccc2cc(CNC3C[C@@H](C)O[C@@H](C)C3)ccc2n1] | | CAS No. 1073-79-6 (Reagent Supplier 3) Cas No. 1369207-07- 7 (Reagent Supplier 9) | 1H NMR (270 MHz) δ 8.06-7.96 (m, 2H), 7.71 (d, J = 1.9 Hz, 1H), 7.65 (dd, J = 8.6, 2.0 Hz, 1H), 7.29 (d, J = 8.3 Hz, 1H), 4.02 (s, 2H), 3.53-3.38 (m, 2H), 2.90-2.70 (m, 4H), 2.00-1.87 (m, 2H), 1.29-0.98 (m, 8H). MS 285.34 (M + H). |
| | | Racemate | | | |
| 77 | tert-butyl 4-(2- methylquinolin-6- yl)piperazine-1- carboxylate | 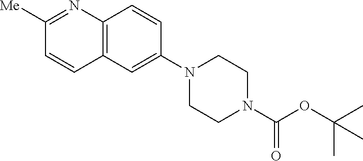 [SMILES: Cc1ccc2cc(N3CCN(C(=O)OC(C)(C)C)CC3)ccc2n1] | | Cas No. 877-42- 9 (Reagent Supplier 3) CAS No. 57260- 71- 6 (Reagent Supplier 7) | 1H NMR (270 MHz) δ 7.94-7.87 (m, 2H), 7.45 (dd, J = 9.3, 2.7 Hz, 1H), 7.22 (d, J = 8.5 Hz, 1H), 7.02 (d, J = 2.7 Hz, 1H), 3.69-3.58 (m, 4H), 3.29- 3.19 (m, 4H), 2.70 (s, 3H), 1.50 (s, 9H). MS 328.37 (M + H). |
| 78 | 2-methyl-6-(4- (oxetan-3- yl)piperazin-1- yl)quinoline | 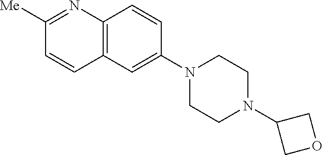 [SMILES: Cc1ccc2cc(N3CCN(C4COC4)CC3)ccc2n1] | | Production Ex. 77 CAS No. 6704- 31-0 (Reagent Supplier 4) | 1H NMR (270 MHz) δ 7.90 (d, J = 8.8 Hz, 2H), 7.46 (dd, J = 9.3, 2.8 Hz, 1H), 7.21 (d, J = 8.3 Hz, 1H), 7.02 (d, J = 2.7 Hz, 1H), 4.79-4.63 (m, 4H), 3.67-3.51 (m, 1H), 3.40- 3.29 (m, 4H), 2.69 (s, 3H), 2.61-2.51 (m, 4H). MS 284.35 (M + H). |
| 79 | N-((2- methylbenzo[d] thiazol-5- yl)methyl) methanesulfonamide | 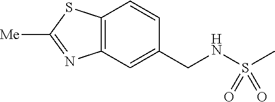 [SMILES: Cc1nc2cc(CNS(C)(=O)=O)ccc2s1] | | CAS No. 20061-46-5 (Reagent Supplier 5) | 1H NMR (270 MHz) δ 7.91 (d, J = 1.7 Hz, 1H), 7.83 (d, J = 8.2 Hz, 1H), 7.38 (dd, J = 8.2, 1.7 Hz, 1H), 4.65 (br s, 1H), 4.47 (d, J = 6.0 Hz, 2H), 2.92 (s, |
| | | | | | 3H), 2.85 (s, 3H). MS |
| | | | | | 257.25 (M + H). |
| 80 | N-((2- methylbenzo[d] thiazol-6- yl)methyl)methane sulfonamide | 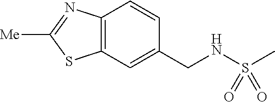 | Production Ex. 79 | CAS No. 20061-51-2 (Reagent Supplier 5) | 1H NMR (270 MHz) δ 7.94 (d, J = 8.4 Hz, 1H), 7.85 (d, J = 1.8 Hz, 1H), 7.42 (dd, J = 8.4, 1.8 Hz, 1H), 4.66 (br s, 1H), 4.45 (d, J = 6.1 Hz, 2H), 2.90 (s, 3H), 2.85 (s, 3H). MS |
| | | | | | 257.21 (M + H). |
| 81 | (4-(2-hydroxy-3- methoxypropyl) piperazin-1-yl)(2- methylquinolin-6- yl)methanone | 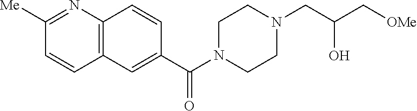 [SMILES: COCC(O)CN1CCN(C(=O)c2ccc3nc(C)ccc3c2)CC1] | | CAS No. 1577354-59-6 (Reagent Supplier 1) CAS No. 930-37- 0 (Reagent | MS 344.39 (M + H). |
| | | | | Supplier 4) | |
| 82 | (4-([1,1′-biphenyl]- 2-yl)-2- methylquinolin-6- yl)(4-(2-hydroxy-3- methoxypropyl) piperazin-1- yl)methanone | 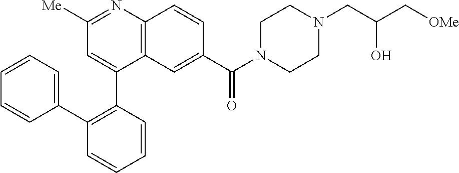 [SMILES: COCC(O)CN1CCN(C(=O)c2ccc3nc(C)cc(-c4ccccc4-c4ccccc4)c3c2)CC1] | Production Ex. 81 | Production Ex. 71 CAS No. 930-37-0 (Reagent Supplier 4) | 1H NMR (500 MHz) δ 7.97 (d, J = 8.6 Hz, 1H), 7.61 (d, J = 1.9 Hz, 1H), 7.57-7.51 (m, 3H), 7.49- 7.44 (m, 1H), 7.36-7.33 (m, 1H), 7.11 (d, J = 1.4 Hz, 1H), 7.07-7.00 (m, 5H), 3.99-3.58 (m, 3H), 3.57-3.09 (m, 7H), 2.78- 2.24 (m, 9H). MS 496.38 (M + H) |
| 83 | ethyl 4-amino-2- methylquinoline-6- carboxylate | 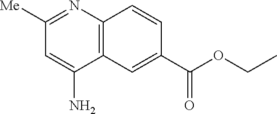 [SMILES: CCOC(=O)c1ccc2nc(C)cc(N)c2c1] | | Production Ex. 66 | 1H NMR (270 MHz) δ 8.54 (d, J = 1.8 Hz, 1H), 8.21 (dd, J = 8.8, 1.9 Hz, 1H), 7.92 (d, J = 8.8 Hz, 1H), 6.55 (s, 1H), 4.83 (br s, 2H), 4.44 (q, J = 7.0 Hz, 2H), 2.61 (s, 3H), 1.44 (t, |
| | | | | | J = 7.1 Hz, 3H). MS 231.20 |
| | | | | | (M + H). |
| 84 | ethyl 4-azido-2- methylquinoline-6- carboxylate | 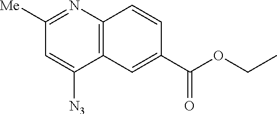 [SMILES: CCOC(=O)c1ccc2nc(C)cc(N=[N+]=[N-])c2c1] | | Production Ex. 66 | 1H NMR (270 MHz) δ 8.77 (d, J = 1.9 Hz, 1H), 8.31 (dd, J = 8.9, 1.9 Hz, 1H), 8.00 (d, J = 8.9 Hz, 1H), 7.09 (s, 1H), 4.45 (q, J = 7.1 Hz, 2H), 2.77 (s, 3H), 1.45 (t, J = 7.1 Hz, |
| | | | | | 3H). MS 257.25 (M + H). |
| 85 | tert-butyl 2- methylquinoline-6- carboxylate | 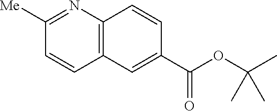 [SMILES: Cc1ccc2cc(C(=O)OC(C)(C)C)ccc2n1] | | CAS No. 635-80-3 (Reagent Supplier 4) | 1H NMR (270 MHz) δ 8.48 (d, J = 2.0 Hz, 1H), 8.23 (dd, J = 8.9, 2.0 Hz, 1H), 8.14 (d, J = 8.3 Hz, 1H), 8.02 (d, J = 8.8 Hz, 1H), 7.35 (d, J = 8.5 Hz, 1H), 2.78 (s, 3H), 1.65 (s, |
| | | | | | 9H). MS 244.27 (M + H). |
| 86 | tert-butyl 4-([1,1′- biphenyl]-2-yl)-2- methylquinoline-6- carboxylate | 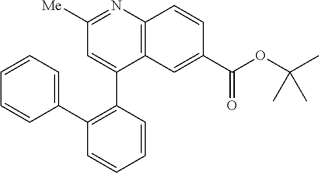 [SMILES: Cc1cc(-c2ccccc2-c2ccccc2)c2cc(C(=O)OC(C)(C)C)ccc2n1] | Production Ex. 85 | Production Ex. 94 | MS 396.31 (M + H) |
| 87 | ethyl 4-cyano-2- methylquinoline-6- carboxylate | 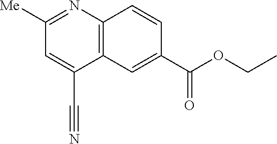 [SMILES: CCOC(=O)c1ccc2nc(C)cc(C#N)c2c1] | | Production Ex. 66 | 1H NMR (270 MHz) δ 8.86 (d, J = 1.9 Hz, 1H), 8.43 (dd, J = 8.8, 1.8 Hz, 1H), 8.15 (d, J = 8.9 Hz, 1H), 7.70 (s, 1H), 4.49 (q, J = 7.0 Hz, 2H), 2.84 (s, 3H), 1.47 (t, J = 7.2 Hz, 3H). MS 241.51 (M + H). |
| 88 | 2-methyl-6- (morpholine-4- carbonyl)quinoline- 4-carbonitrile | 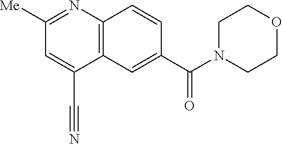 [SMILES: Cc1cc(C#N)c2cc(C(=O)N3CCOCC3)ccc2n1] | Production Ex. 87 | Production Ex. 34 | 1H NMR (270 MHz) δ 8.20 (d, J = 1.8 Hz, 1H), 8.16 (d, J = 8.8 Hz, 1H), 7.84 (dd, J = 8.6, 1.9 Hz, 1H), 7.69 (s, 1H), 3.94- 3.41 (m, 8H), 2.83 (s, 3H). MS 282.12 (M + H). |
| 89 | 2-(3-methoxy-4-(2- morpholino-2- oxoethoxy) benzylidene) indolin-3-one | 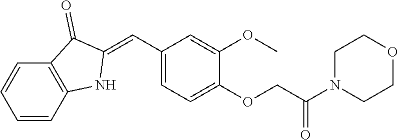 [SMILES: COc1cc(/C=C2\Nc3ccccc3C2=O)ccc1OCC(=O)N1CCOCC1] | | Example 2 | MS 395.18 (M + H). |
| 90 | 4-azido-2- methylquinoline-6- carboxylic acid | 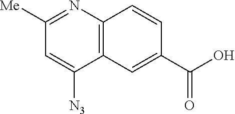 [SMILES: Cc1cc(N=[N+]=[N-])c2cc(C(=O)O)ccc2n1] | | Production Ex. 84 | MS 229.18 (M + H). |
| 91 | 4-carbamoyl-2- methylquinoline-6- carboxylic acid | 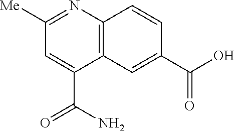 [SMILES: Cc1cc(C(N)=O)c2cc(C(=O)O)ccc2n1] | Production Ex. 90 | Production Ex. 87 | MS 231.12 (M + H). |
| 92 | 4-chloro-2- methylquinoline-6- carboxylic acid | 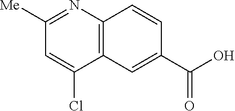 [SMILES: Cc1cc(Cl)c2cc(C(=O)O)ccc2n1] | Production Ex. 90 | Production Ex. 66 | 1H NMR (270 MHz, dimethylsulfoxide-d6) 8 13.46 (br s, 1H), 8.75 (d, J = 1.9 Hz, 1H), 8.27 (dd, J = 8.9, 1.9 Hz, 1H), 8.08 (d, J = 8.7 Hz, 1H), 7.83 (s, 1H), 2.70 (s, 3H). MS 222.00 |
| | | | | | (M + H). |
| 93 | 4-amino-2- methylquinoline-6- carboxylic acid dihydrochloride | 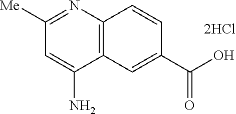 [SMILES: Cc1cc(N)c2cc(C(=O)O)ccc2n1] | | Production Ex. 83 | MS 203.12 (M + H). |
| 94 | 4-([1,1′-biphenyl]- 2-yl)-2- methylquinoline-6- carboxylic acid | 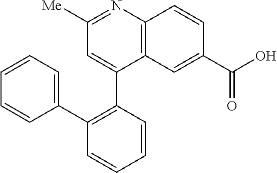 [SMILES: Cc1cc(-c2ccccc2-c2ccccc2)c2cc(C(=O)O)ccc2n1] | | Production Ex. 106 | 1H NMR (500 MHz, dimethylsulfoxide-d6) δ 8.07 (d, J = 1.8 Hz, 1H), 8.03 (dd, J = 8.7, 1.9 Hz, 1H), 7.91 (d, J = 8.7 Hz, 1H), 7.70-7.64 (m, 1H), 7.63-7.56 (m, 2H), 7.50- 7.46 (m, 1H), 7.39 (s, 1H), 7.10-7.00 (m, 5H), 2.63 (s, 3H). MS 340.27 (M + H). |
| 95 | (2-methyl-4- phenylquinolin-6- yl)(morpholino) methanone | 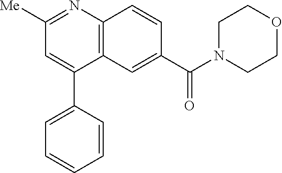 [SMILES: Cc1cc(-c2ccccc2)c2cc(C(=O)N3CCOCC3)ccc2n1] | | Production Ex. 34 | 1H NMR (270 MHz) δ 8.12 (d, J = 8.6 Hz, 1H), 7.94 (d, J = 1.9 Hz, 1H), 7.70 (dd, J = 8.6, 1.8 Hz, 1H), 7.58-7.42 (m, 5H), 7.30 (s, 1H), 4.05-3.33 (m, 8H), 2.80 (s, 3H). MS 333.28 (M + H). |
| 96 | (4-butoxy-2- methylquinolin-6- yl)(morpholino) methanone | 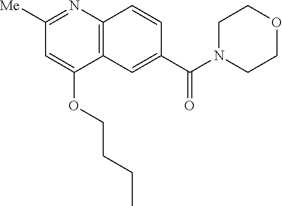 [SMILES: CCCCOc1cc(C)nc2ccc(C(=O)N3CCOCC3)cc12] | | Production Ex. 34 | 1H NMR (270 MHz) δ 8.26 (d, J = 2.0 Hz, 1H), 7.96 (d, J = 8.6 Hz, 1H), 7.66 (dd, J = 8.6, 2.0 Hz, 1H), 6.66 (s, 1H), 4.18 (t, J = 6.4 Hz, 2H), 3.94-3.40 (m, 8H), 2.70 (s, 3H), 1.99- 1.84 (m, 2H), 1.69-1.49 (m, 2H), 1.03 (t, J = 7.3 Hz, 3H). MS 329.24 (M + H). |
| 97 | (2-methyl-4- (pyridin-3- yl)quinolin-6- yl)(morpholino) methanone | 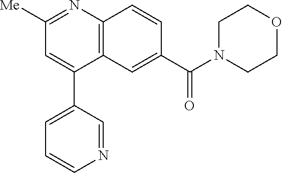 [SMILES: Cc1cc(-c2cccnc2)c2cc(C(=O)N3CCOCC3)ccc2n1] | | Production Ex. 34 | 1H NMR (270 MHz) δ 8.81-8.72 (m, 2H), 8.15 (d, J = 8.6 Hz, 1H), 7.87- 7.80 (m, 2H), 7.73 (dd, J = 8.6, 1.8 Hz, 1H), 7.50 (dd, J = 7.8, 4.8 Hz, 1H), 7.31 (s, 1H), 3.97-3.34 (m, 8H), 2.82 (s, 3H). MS 334.23 (M + H). |
| 98 | (4-([1,1′-biphenyl]- 2-yl)-2- methylquinolin-6- yl)(morpholino) methanone | 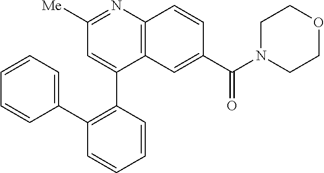 [SMILES: Cc1cc(-c2ccccc2-c2ccccc2)c2cc(C(=O)N3CCOCC3)ccc2n1] | | Production Ex. 34 | 1H NMR (270 MHz) δ 7.99 (d, J = 8.6 Hz, 1H), 7.64-7.44 (m, 5H), 7.39- 7.33 (m, 1H), 7.15 (s, 1H), 7.07-7.03 (m, 5H), 3.97- 3.11 (m, 8H), 2.67 (s, 3H). MS 409.34 (M + H). |
| 99 | (4-([1,1′-biphenyl]- 3-yl)-2- methylquinolin-6- yl)(morpholino) methanone | 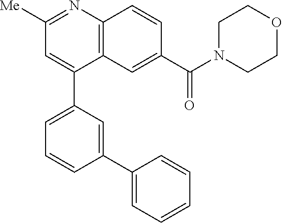 [SMILES: Cc1cc(-c2cccc(-c3ccccc3)c2)c2cc(C(=O)N3CCOCC3)ccc2n1] | Production Ex. 98 | Production Ex. 34 | MS 409.34 (M + H). |
| 100 | (2-methyl-[4,8′- biquinolin]-6- yl)(morpholino) methanone | 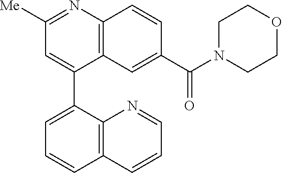 [SMILES: Cc1cc(-c2cccc3cccnc23)c2cc(C(=O)N3CCOCC3)ccc2n1] | Production Ex. 98 | Production Ex. 34 | 1H NMR (270 MHz) δ 8.78-8.72 (m, 1H), 8.31 (dd, J = 8.4, 1.8 Hz, 1H), 8.15 (d, J = 8.6 Hz, 1H), 8.06-7.97 (m, 1H), 7.77- 7.64 (m, 3H), 7.50-7.42 (m, 1H), 7.41 (s, 1H), 7.35 (d, J = 1.9 Hz, 1H), 3.83- 3.09 (m, 8H), 2.83 (s, 3H). MS 384.33 (M + H). |
| 101 | (2-methyl-4-(4- phenoxyphenyl) quinolin-6- yl)(morpholino) methanone | 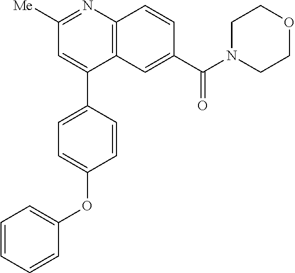 [SMILES: Cc1cc(-c2ccc(Oc3ccccc3)cc2)c2cc(C(=O)N3CCOCC3)ccc2n1] | Production Ex. 98 | Production Ex. 34 | 1H NMR (270 MHz) δ 8.11 (d, J = 8.6 Hz, 1H), 8.00 (d, J = 1.9 Hz, 1H), 7.69 (dd, J = 8.6, 1.9 Hz, 1H), 7.50-7.35 (m, 4H), 7.29 (s, 1H), 7.24-7.09 (m, 5H), 3.88-3.35 (m, 8H), 2.79 (s, 3H). MS 425.37 (M + H). |
| 102 | (2-methyl-[4,4′- biquinolin]-6- yl)(morpholino) methanone | 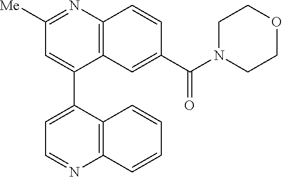 [SMILES: Cc1cc(-c2ccnc3ccccc23)c2cc(C(=O)N3CCOCC3)ccc2n1] | Production Ex. 98 | Production Ex. 34 | 1H NMR (270 MHz) δ 9.08 (d, J = 4.3 Hz, 1H), 8.27 (d, J = 8.5 Hz, 1H), 8.19 (d, J = 8.6 Hz, 1H), 7.83-7.70 (m, 2H), 7.65- 7.29 (m, 5H), 3.84-3.09 (m, 8H), 2.85 (s, 3H). MS 384.33 (M + H). |
| 103 | (2-methyl-4- (naphthalen-1- yl)quinolin-6- yl)(morpholino) methanone | 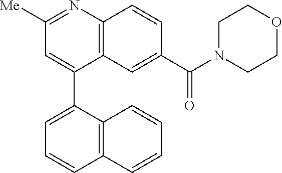 [SMILES: Cc1cc(-c2cccc3ccccc23)c2cc(C(=O)N3CCOCC3)ccc2n1] | Production Ex. 98 | Production Ex. 34 | 1H NMR (270 MHz) δ 8.17 (d, J = 8.6 Hz, 1H), 8.04-7.94 (m, 2H), 7.74 (dd, J = 8.6, 1.9 Hz, 1H), 7.62 (dd, J = 8.3, 7.1 Hz, 1H), 7.55-7.43 (m, 2H), 7.40 (s, 1H), 7.35 (d, J = 1.9 Hz, 1H), 7.33-7.28 (m, 2H), 3.86-3.05 (m, 8H), 2.84 (s, 3H). MS 383.33 (M + H). |
| 104 | (2′-methyl-[3,4′- biquinolin]-6′- yl)(morpholino) methanone | 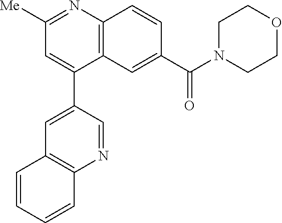 [SMILES: Cc1cc(-c2cnc3ccccc3c2)c2cc(C(=O)N3CCOCC3)ccc2n1] | Production Ex. 98 | Production Ex. 34 | 1H NMR (500 MHz) δ 9.01 (d, J = 2.3 Hz, 1H), 8.31-8.29 (m, 1H), 8.23- 8.20 (m, 1H), 8.17-8.15 (m, 1H), 7.93-7.90 (m, 1H), 7.88-7.87 (m, 1H), 7.86-7.82 (m, 1H), 7.73 (dd, J = 8.6, 1.8 Hz, 1H), 7.69-7.64 (m, 1H), 7.39 (s, 1H), 3.86-3.29 (m, 8H), 2.84 (s, 3H). MS 384.46 (M + H). |
| 105 | (2-methyl-4-(2- phenylpyridin-3- yl)quinolin-6- yl)(morpholino) methanone | 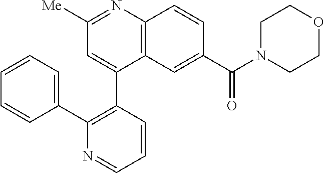 [SMILES: Cc1cc(-c2cccnc2-c2ccccc2)c2cc(C(=O)N3CCOCC3)ccc2n1] | Production Ex. 98 | Production Ex. 34 | 1H NMR (500 MHz) δ 8.84 (dd, J = 4.8, 1.7 Hz, 1H), 8.01 (d, J = 8.6 Hz, 1H), 7.69 (dt, J = 7.7, 1.9 Hz, 1H), 7.56 (dt, J = 8.6, 2.0 Hz, 1H), 7.47 (d, J = 1.8 Hz, 1H), 7.45-7.37 (m, 1H), 7.33-7.26 (m, 2H), 7.21 (s, 1H), 7.13- 7.02 (m, 3H), 3.89-3.01 (m, 8H), 2.71 (s, 3H). MS |
| | | | | | 410.28 (M + H). |
| 106 | ethyl 4-([1,1′- biphenyl]-2-yl)-2- methylquinoline-6- carboxylate | 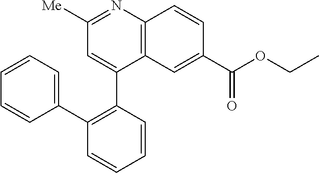 [SMILES: CCOC(=O)c1ccc2nc(C)cc(-c3ccccc3-c3ccccc3)c2c1] | Production Ex. 98 | Production Ex. 66 | 1H NMR (500 MHz) δ 8.31 (d, J = 1.9 Hz, 1H), 8.14 (dd, J = 8.8, 1.9 Hz, 1H), 7.96 (dd, J = 8.8, 0.6 Hz, 1H), 7.61-7.37 (m, 4H), 7.12 (s, 1H), 7.06- 6.97 (m, 5H), 4.43-4.27 (m, 2H), 2.66 (s, 3H), 1.38 (t, J = 7.1 Hz, 3H). MS 368.25 (M + H). |
| 107 | (4-chloro-2- (hydroxymethyl) quinolin-6- yl)(morpholino) methanone | 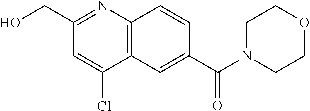 [SMILES: O=C(c1ccc2nc(CO)cc(Cl)c2c1)N1CCOCC1] | | Production Ex. 153 | 1H NMR (270 MHz) δ 8.30 (d, J = 2.0 Hz, 1H), 8.14 (d, J = 8.6 Hz, 1H), 7.80 (dd, J = 8.7, 1.8 Hz, 1H), 7.49 (s, 1H), 4.92 (d, J = 4.9 Hz, 2H), 4.10-3.40 (m, 9H). MS 307.13 |
| | | | | | (M + H). |
| 108 | (2-(((tert- butyldimethylsilyl) oxy)methyl)-4- chloroquinolin-6- yl)(morpholino) methanone | 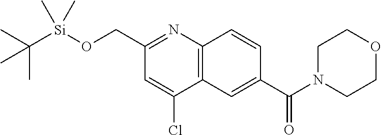 [SMILES: CC(C)(C)[Si](C)(C)OCc1cc(Cl)c2cc(C(=O)N3CCOCC3)ccc2n1] | | Production Ex. 107 | 1H NMR (270 MHz) 8.30 (d, J = 1.9 Hz, 1H), 8.06 (d, J = 8.6 Hz, 1H), 7.84 (s, 1H), 7.76 (dd, J = 8.7, 1.8 Hz, 1H), 4.98 (s, 2H), 3.95-3.45 (m, 8H), 0.99 (s, 9H), 0.16 (s, 6H). MS 421.29 (M + H). |
| 109 | 1-(2-methyl-6- (morpholine-4- carbonyl)quinolin- 4-yl)piperidine-4- carbonitrile | 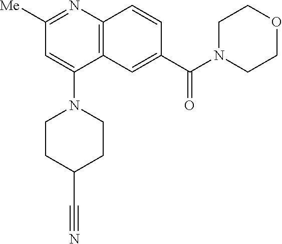 [SMILES: Cc1cc(N2CCC(C#N)CC2)c2cc(C(=O)N3CCOCC3)ccc2n1] | | Production Ex. 34 CAS No. 4395- 98-6 (Reagent Supplier 9) | 1H NMR (270 MHz) δ 8.03-7.97 (m, 2H), 7.63 (dd, J = 8.7, 1.8 Hz, 1H), 6.82 (s, 1H), 3.93-3.33 (m, 10H), 3.24-3.09 (m, 2H), 3.01-2.88 (m, 1H), 2.70 (s, 3H), 2.30-2.07 (m, 4H). MS 365.33 (M + H). |
| 110 | 1-(4-(2-(((tert- butyldimethylsilyl) oxy)methyl)-6- (morpholine-4- carbonyl)quinolin- 4-yl)piperazin-1- yl)ethan-1-one | 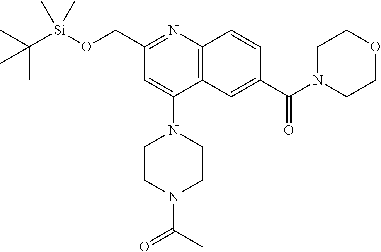 [SMILES: CC(=O)N1CCN(c2cc(CO[Si](C)(C)C(C)(C)C)nc3ccc(C(=O)N4CCOCC4)cc23)CC1] | Production Ex. 109 | Production Ex. 108 | 1H NMR (270 MHz) δ 8.10 (d, J = 1.8 Hz, 1H), 8.00 (d, J = 8.6 Hz, 1H), 7.65 (dd, J = 8.7, 1.9 Hz, 1H), 7.26 (s, 1H), 4.94 (s, 2H), 3.97-3.55 (m, 12H), 3.30-3.16 (m, 4H), 2.19 (s, 3H), 0.99 (s, 9H), 0.15 (s, 6H). MS 513.45 (M + H). |
| 111 | 1-(4-(2- (hydroxymethyl)- 6-(morpholine-4- carbonyl)quinolin- 4-yl)piperazin-1- yl)ethan-1-one | 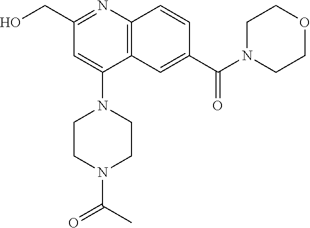 [SMILES: CC(=O)N1CCN(c2cc(CO)nc3ccc(C(=O)N4CCOCC4)cc23)CC1] | | Production Ex. 110 | 1H NMR (270 MHz) δ 8.13-8.01 (m, 2H), 7.67 (dd, J = 8.6, 1.9 Hz, 1H), 6.82 (s, 1H), 4.86 (s, 2H), 4.40 (br s, 1H), 3.97-3.36 (m, 12H), 3.28-3.13 (m, 4H), 2.18 (s, 3H). MS 399.40 (M + H). |
| 112 | tert-butyl ((2- methylquinolin-6- yl)methyl) (tetrahydro- 2H-pyran-4- yl)carbamate | 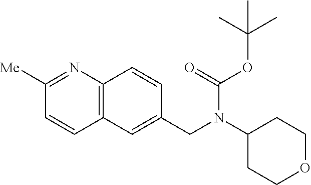 [SMILES: Cc1ccc2cc(CN(C(=O)OC(C)(C)C)C3CCOCC3)ccc2n1] | | CAS No. 1981263- 96-0 (Reagent Supplier 1) | 1H NMR (270 MHz) δ 8.04-7.92 (m, 2H), 7.64- 7.52 (m, 2H), 7.33-7.25 (m, 1H), 4.57 (s, 2H), 4.00- 3.89 (m, 2H), 3.50-3.28 (m, 2H), 2.74 (s, 3H), 1.86- 1.19 (m, 14H). MS 357.34 (M + H). |
| 113 | tert-butyl ((2- methylbenzo[d] thiazol-5- yl)methyl) (tetrahydro-2H- pyran-4- yl)carbamate | 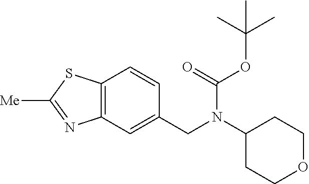 [SMILES: Cc1nc2cc(CN(C(=O)OC(C)(C)C)C3CCOCC3)ccc2s1] | Production Ex. 112 | Production Ex. 69 | 1H NMR (270 MHz) δ 7.80 (d, J = 1.5 Hz, 1H), 7.75 (d, J = 8.3 Hz, 1H), 7.28-7.21 (m, 1H), 4.54 (s, 2H), 3.99-3.88 (m, 2H), 3.47-3.30 (m, 2H), 2.83 (s, 3H), 1.78-1.31 (m, 14H). MS 363.32 (M + H). |
| 114 | tert-butyl (1,1- dioxidotetrahydro- 2H-thiopyran-4- yl)((2- methylquinolin-6- yl)methyl) carbamate | 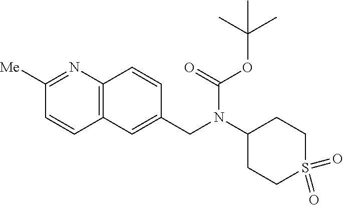 [SMILES: Cc1ccc2cc(CN(C(=O)OC(C)(C)C)C3CCS(=O)(=O)CC3)ccc2n1] | Production Ex. 112 | CAS No. 1980996- 27-7 (Reagent Supplier 1) | 1H NMR (270 MHz) δ 8.03-7.95 (m, 2H), 7.61- 7.51 (m, 2H), 7.31 (d, J = 8.4 Hz, 1H), 4.58 (s, 2H), 3.13-2.92 (m, 4H), 2.75 (s, 3H), 2.41-2.23 (m, 1H), 2.08-1.92 (m, 2H), 1.67-1.33 (m, 11H). MS 405.33 (M + H). |
| 115 | tert-butyl ((2- methylquinolin-6- yl)methyl)(1- (oxetan-3- yl)piperidin-4- yl)carbamate | 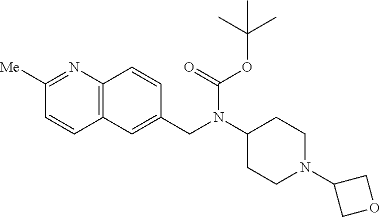 [SMILES: Cc1ccc2cc(CN(C(=O)OC(C)(C)C)C3CCN(C4COC4)CC3)ccc2n1] | Production Ex. 112 | Production Ex. 70 | 1H NMR (270 MHz) δ 8.03-7.91 (m, 2H), 7.60- 7.51 (m, 2H), 7.28 (d, J = 8.4 Hz, 1H), 4.67-4.47 (m, 6H), 3.49-3.33 (m, 1H), 2.79-2.67 (m, 5H), 1.98-1.18 (m, 16H). MS 412.52 (M + H). |
| 116 | tert-butyl ((2- methylbenzo[d] thiazol-6- yl)methyl) (tetrahydro- 2H-pyran-4- yl)carbamate | 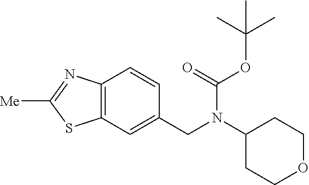 [SMILES: Cc1nc2ccc(CN(C(=O)OC(C)(C)C)C3CCOCC3)cc2s1] | Production Ex. 112 | Production Ex. 68 | 1H NMR (270 MHz) δ 7.87 (d, J = 8.5 Hz, 1H), 7.70-7.63 (m, 1H), 7.34- 7.28 (m, 1H), 4.51 (s, 2H), 4.00-3.89 (m, 2H), 3.46- 3.32 (m, 2H), 2.83 (s, 3H), 1.79-1.29 (m, 14H). MS 363.41 (M + H). |
| 117 | tert-butyl ((2SR,6RS)-2,6- dimethyltetrahydro- 2H-pyran-4-yl)((2- methylquinolin-6- yl)methyl)carbamate | 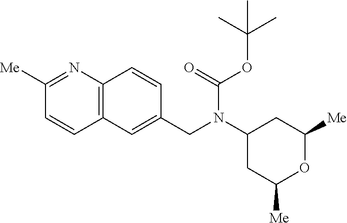 [SMILES: Cc1ccc2cc(CN(C(=O)OC(C)(C)C)C3C[C@@H](C)O[C@@H](C)C3)ccc2n1] | Production Ex. 112 | Production Ex. 76 | 1H NMR (270 MHz) δ 8.04-7.92 (m, 2H), 7.60- 7.51 (m, 2H), 7.29 (d, J = 8.4 Hz, 1H), 4.52 (s, 2H), 3.60-3.40 (m, 2H), 2.74 (s, 3H), 1.75-1.21 (m, 14H), 1.16 (s, 3H), 1.14 (s, 3H). MS 385.36 (M + H). |
| | | Racemate | | | |
| 118 | tert-butyl ((2SR,6SR)-2,6- dimethyltetrahydro- 2H-pyran-4-yl)((2- methylquinolin-6- yl)methyl)carbamate | 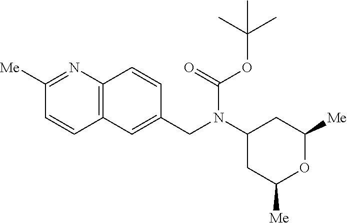 [SMILES: Cc1ccc2cc(CN(C(=O)OC(C)(C)C)C3C[C@@H](C)O[C@@H](C)C3)ccc2n1] | Production Ex. 112 | Production Ex. 75 | 1H NMR (270 MHz) δ 8.02-7.95 (m, 2H), 7.53- 7.47 (m, 2H), 7.29 (d, J = 8.4 Hz, 1H), 4.77 (s, 2H), 4.42-4.35 (m, 1H), 3.81- 3.67 (m, 2H), 2.74 (s, 3H), 1.96-1.84 (m, 2H), 1.57- 1.43 (m, 2H), 1.39 (s, 9H), 1.12 (s, 3H), 1.10 (s, 3H). MS 385.40 (M + H). |
| | | Racemate | | | |
| 119 | 6-(morpholine-4- carbonyl)quinoline- 2-carbaldehyde | 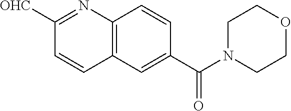 [SMILES: O=Cc1ccc2cc(C(=O)N3CCOCC3)ccc2n1] | | Production Ex. 19 | 1H NMR (270 MHz) δ 10.24 (s, 1H), 8.42-8.27 (m, 2H), 8.10 (d, J = 8.5 Hz, 1H), 7.99 (d, J = 1.8 Hz, 1H), 7.83 (dd, J = 8.7, 1.9 Hz, 1H), 4.00-3.41 (m, 8H). MS 270.94 (M + H). |
| 120 | 6- (hydroxymethyl) quinoline-2- carbaldehyde | 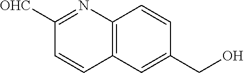 [SMILES: O=Cc1ccc2cc(CO)ccc2n1] | Production Ex. 119 | CAS No. 108166-02-5 (Reagent Supplier 8) | 1H NMR (270 MHz) δ 10.23 (s, 1H), 8.36-8.20 (m, 2H), 8.05 (d, J = 8.5 Hz, 1H), 7.95-7.88 (m, 1H), 7.81 (dd, J = 8.7, 1.9 |
| | | | | | Hz, 1H), 4.97 (d, J = 5.2 |
| | | | | | Hz, 2H), 1.95 (t, J = 5.8 |
| | | | | | Hz, 1H). MS 188.18 |
| | | | | | (M + H). |
| 121 | 6- (morpholinomethyl) quinoline-2- carbaldehyde | 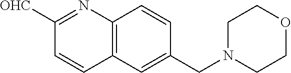 [SMILES: O=Cc1ccc2cc(CN3CCOCC3)ccc2n1] | Production Ex. 119 | Production Ex. 15 | 1H NMR (270 MHz) δ 10.23 (d, J = 1.0 Hz, 1H), 8.29 (d, J = 8.4 Hz, 1H), 8.22 (d, J = 8.6 Hz, 1H), 8.04 (d, J = 8.5 Hz, 1H), |
| | | | | | 7.93-7.80 (m, 2H), 3.80- |
| | | | | | 3.69 (m, 6H), 2.57-2.47 |
| | | | | | (m, 4H). MS 257.21 |
| | | | | | (M + H). |
| 122 | 7-(morpholine-4- carbonyl)quinoline- 2-carbaldehyde | 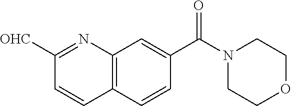 [SMILES: O=Cc1ccc2ccc(C(=O)N3CCOCC3)cc2n1] | Production Ex. 119 | CAS No. 1384433- 28-6 (Reagent Supplier 14) | 1H NMR (270 MHz) δ 10.27-10.20 (m, 1H), 8.36 (d, J = 8.4 Hz, 1H), 8.30- 8.22 (m, 1H), 8.10 (d, J = 8.5 Hz, 1H), 8.00 (d, J = 8.6 Hz, 1H), 7.76 (dd, J = 8.4, 1.7 Hz, 1H), 3.76 (s, |
| | | | | | 8H). MS 271.30 (M + H). |
| 123 | N-((2- formylquinolin-6- yl)methyl)acetamide | 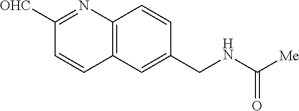 [SMILES: CC(=O)NCc1ccc2nc(C=O)ccc2c1] | Production Ex. 119 | Production Ex. 48 | 1H NMR (270 MHz) δ 10.22 (d, J = 0.8 Hz, 1H), 8.33-8.18 (m, 2H), 8.04 (d, J = 8.5 Hz, 1H), 7.83- 7.70 (m, 2H), 5.97 (br s, 1H), 4.68 (d, J = 6.0 Hz, 2H), 2.11 (s, 3H). MS 247.26 (M + H₂O + H). |
| 124 | N-((2- formylquinolin-6- yl)methyl)methane sulfonamide | 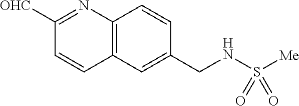 [SMILES: CS(=O)(=O)NCc1ccc2nc(C=O)ccc2c1] | Production Ex. 119 | Production Ex. 59 | MS 283.24 (M + H₂O + H). |
| 125 | 6-(2-morpholino-2- oxoethoxy)quinoline- 2-carbaldehyde | 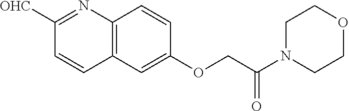 [SMILES: O=Cc1ccc2cc(OCC(=O)N3CCOCC3)ccc2n1] | Production Ex. 119 | Production Ex. 53 | 1H NMR (270 MHz) δ 10.19 (s, 1H), 8.26-8.13 (m, 2H), 8.02 (d, J = 8.5 Hz, 1H), 7.52 (dd, J = 9.3, 2.8 Hz, 1H), 7.25 (d, J = 2.9 Hz, 1H), 4.88 (s, 2H), 3.68 (m, 8H). MS 301.32 |
| | | | | | (M + H). |
| 126 | 6-(morpholine-4- carbonyl)benzo[d] thiazole-2- carbaldehyde | 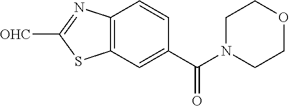 [SMILES: O=Cc1nc2ccc(C(=O)N3CCOCC3)cc2s1] | Production Ex. 119 | CAS No. 878973-93-4 (Reagent Supplier 1) | 1H NMR (500 MHz) δ 10.18 (s, 1H), 8.29 (d, J = 8.5 Hz, 1H), 8.10 (d, J = 1.6 Hz, 1H), 7.64 (dd, J = 8.4, 1.6 Hz, 1H), 3.90- 3.61 (m, 8H). MS 277.24 (M + H). |
| 127 | 5-(morpholine-4- carbonyl)thiazole- 2-carbaldehyde | 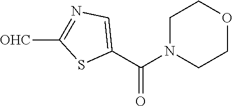 [SMILES: O=Cc1ncc(C(=O)N2CCOCC2)s1] | Production Ex. 119 | CAS No. 79836-80-9 (Reagent Supplier 1) | MS 227.14 (M + H). |
| 128 | 6-(4- phenylpiperidine-1- carbonyl)quinoline- 2-carbaldehyde | 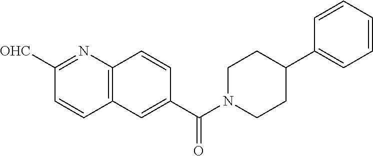 [SMILES: O=Cc1ccc2cc(C(=O)N3CCC(c4ccccc4)CC3)ccc2n1] | Production Ex. 119 | Production Ex. 46 | MS 345.35 (M + H). |
| 129 | 6-(4- morpholinopiperidine- 1-carbonyl)quinoline- 2-carbaldehyde | 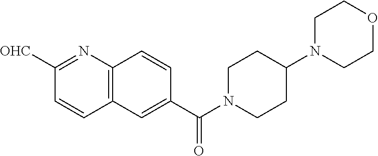 [SMILES: O=Cc1ccc2cc(C(=O)N3CCC(N4CCOCC4)CC3)ccc2n1] | Production Ex. 119 | Production Ex. 37 | MS 354.40 (M + H). |
| 130 | 6-(4-(2- methoxyethyl)pipe razine-1- carbonyl)quinoline- 2-carbaldehyde | 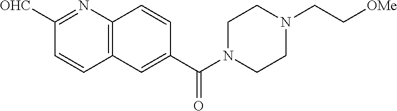 [SMILES: COCCN1CCN(C(=O)c2ccc3nc(C=O)ccc3c2)CC1] | Production Ex. 119 | Production Ex. 38 | 1H NMR (270 MHz) δ 10.24 (s, 1H), 8.41-8.25 (m, 2H), 8.09 (d, J = 8.4 Hz, 1H), 7.98 (d, J = 1.8 Hz, 1H), 7.83 (dd, J = 8.7, 1.8 Hz, 1H), 3.98-3.79 (m, 2H), 3.60-3.45 (m, |
| | | | | | 4H), 3.36 (s, 3H), 2.69- |
| | | | | | 2.43 (m, 6H). MS 328.38 |
| | | | | | (M + H). |
| 131 | 5-(morpholine-4- carbonyl)benzo[d] thiazole-2- carbaldehyde | 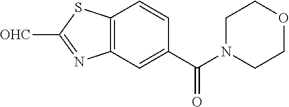 [SMILES: O=Cc1nc2cc(C(=O)N3CCOCC3)ccc2s1] | Production Ex. 119 | Production Ex. 39 | 1H NMR (270 MHz) δ 10.19 (s, 1H), 8.28 (d, J = 1.5 Hz, 1H), 8.09 (d, J = 8.3 Hz, 1H), 7.66 (dd, J = 8.3, 1.6 Hz, 1H), 3.98- 3.39 (m, 8H). MS 277.20 (M + H). |
| 132 | 6-(1,1- dioxidothiomorpholine- 4-carbonyl) quinoline- 2-carbaldehyde | 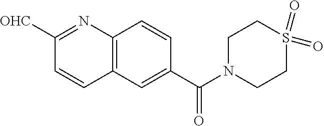 [SMILES: O=Cc1ccc2cc(C(=O)N3CCS(=O)(=O)CC3)ccc2n1] | Production Ex. 119 | Production Ex. 41 | 1H NMR (270 MHz) δ 10.25 (d, J = 0.9 Hz, 1H), 8.44-8.31 (m, 2H), 8.12 (d, J = 8.4 Hz, 1H), 8.04 (d, J = 1.9 Hz, 1H), 7.84 (dd, J = 8.8, 1.9 Hz, 1H), 4.44- 3.92 (m, 4H), 3.34-2.90 (m, 4H). MS 319.30 |
| | | | | | (M + H). |
| 133 | 6-((1,1- dioxidothiomorpholino) methyl)quinoline- 2-carbaldehyde | 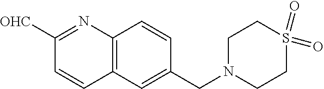 [SMILES: O=Cc1ccc2cc(CN3CCS(=O)(=O)CC3)ccc2n1] | Production Ex. 119 | Production Ex. 14 | 1H NMR (270 MHz) δ 10.23 (d, J = 1.0 Hz, 1H), 8.35-8.20 (m, 2H), 8.06 (d, J = 8.5 Hz, 1H), 7.90- 7.79 (m, 2H), 3.88 (s, 2H), 3.17-3.01 (m, 8H). MS |
| | | | | | 305.26 (M + H). |
| 134 | 6- ((methyl(tetrahydro- 2H-pyran-4- yl)amino)methyl) quinoline-2- carbaldehyde | 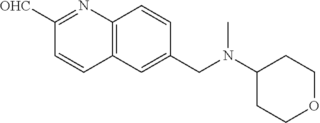 [SMILES: CN(Cc1ccc2nc(C=O)ccc2c1)C1CCOCC1] | Production Ex. 119 | Production Ex. 67 | 1H NMR (270 MHz) δ 10.23 (s, 1H), 8.33-8.16 (m, 2H), 8.03 (d, J = 8.5 Hz, 1H), 7.91-7.80 (m, 2H), 4.14-4.01 (m, 2H), 3.80 (s, 2H), 3.40 (td, J = 11.5, 2.6 Hz, 2H), 2.81- 2.63 (m, 1H), 2.26 (s, 3H), |
| | | | | | 1.88-1.64 (m, 4H). MS |
| | | | | | 285.28 (M + H). |
| 135 | 6-(4-(oxetan-3- yl)piperazine-1- carbonyl)quinoline- 2-carbaldehyde | 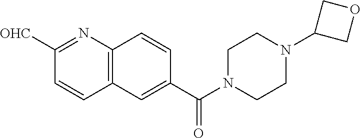 [SMILES: O=Cc1ccc2cc(C(=O)N3CCN(C4COC4)CC3)ccc2n1] | Production Ex. 119 | Production Ex. 72 | 1H NMR (270 MHz) δ 10.24 (d, J = 0.9 Hz, 1H), 8.41-8.26 (m, 2H), 8.09 (d, J = 8.5 Hz, 1H), 7.98 (d, J = 1.9 Hz, 1H), 7.82 (dd, J = 8.7, 1.8 Hz, 1H), 4.75- 4.56 (m, 4H), 3.97-3.41 (m, 5H), 2.52-2.22 (m, 4H). MS 326.30 (M + H). |
| 136 | 6-(4-(2-hydroxy-3- methoxypropyl) piperazine-1- carbonyl)quinoline- 2-carbaldehyde | 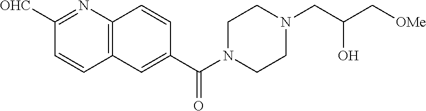 [SMILES: COCC(O)CN1CCN(C(=O)c2ccc3nc(C=O)ccc3c2)CC1] | Production Ex. 119 | Production Ex. 81 | 1H NMR (270 MHz) δ 10.24 (d, J = 0.9 Hz, 1H), 8.41-8.27 (m, 2H), 8.10 (d, J = 8.5 Hz, 1H), 7.99 (d, J = 1.9 Hz, 1H), 7.83 (dd, J = 8.6, 1.9 Hz, 1H), 4.11- 3.23 (m, 12H), 2.83-2.39 |
| | | | | | (m, 4H). MS 358.42 |
| | | | | | (M + H). |
| 137 | (E)-6-(2- (morpholinosulfonyl) vinyl)benzo[d] thiazole-2- carbaldehyde | 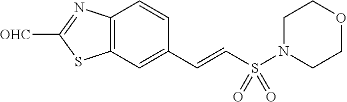 [SMILES: O=Cc1nc2ccc(/C=C/S(=O)(=O)N3CCOCC3)cc2s1] | Production Ex. 119 | Production Ex. 4 | MS 339.16 (M + H). |
| 138 | 6-(4-(oxetan-3- yl)piperazin-1- yl)quinoline-2- carbaldehyde | 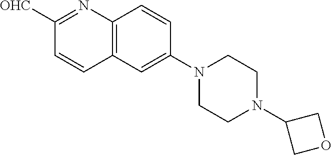 [SMILES: O=Cc1ccc2cc(N3CCN(C4COC4)CC3)ccc2n1] | Production Ex. 119 | Production Ex. 78 | 1H NMR (270 MHz) δ 10.15 (s, 1H), 8.08 (dd, J = 8.9, 5.0 Hz, 2H), 7.94 (d, J = 8.4 Hz, 1H), 7.57 (dd, J = 9.4, 2.8 Hz, 1H), 7.04 (d, J = 2.8 Hz, 1H), 4.79-4.62 (m, 4H), 3.68-3.52 (m, 1H), 3.52-3.35 (m, 4H), 2.62-2.52 (m, 4H). MS 298.37 (M + H). |
| 139 | (E)-5-(3- morpholino-3- oxoprop-1-en-1- yl)benzo[d]thiazole- 2-carbaldehyde | 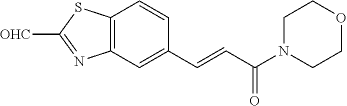 [SMILES: O=Cc1nc2cc(/C=C/C(=O)N3CCOCC3)ccc2s1] | Production Ex. 119 | Production Ex. 23 | 1H NMR (270 MHz) δ 10.18 (s, 1H), 8.37 (d, J = 1.6 Hz, 1H), 8.02 (d, J = 8.5 Hz, 1H), 7.86 (d, J = 15.3 Hz, 1H), 7.75 (dd, J = 8.5, 1.7 Hz, 1H), 7.00 (d, J = 15.5 Hz, 1H), 3.86-3.63 |
| | | | | | (m, 8H). MS 303.30 |
| | | | | | (M + H). |
| 140 | 6-((4-(oxetan-3- yl)piperazin-1- yl)methyl)quinoline- 2-carbaldehyde | 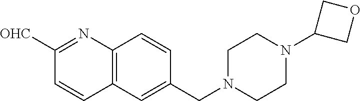 [SMILES: O=Cc1ccc2cc(CN3CCN(C4COC4)CC3)ccc2n1] | Production Ex. 119 | Production Ex. 73 | 1H NMR (270 MHz) δ 10.23 (d, J = 0.8 Hz, 1H), 8.33-8.16 (m, 2H), 8.03 (d, J = 8.3 Hz, 1H), 7.91- 7.79 (m, 2H), 4.72-4.57 (m, 4H), 3.74 (s, 2H), 3.61- |
| | | | | | 3.45 (m, 1H), 2.67-2.51 |
| | | | | | (m, 4H), 2.48-2.31 (m, |
| | | | | | 4H). MS 312.35 (M + H). |
| 141 | N-((2- formylbenzo[d] thiazo1-5- yl)methyl)methane sulfonamide | 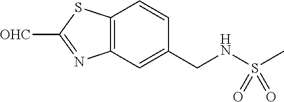 [SMILES: CS(=O)(=O)NCc1ccc2sc(C=O)nc2c1] | Production Ex. 119 | Production Ex. 79 | 1H NMR (270 MHz) δ 10.17 (s, 1H), 8.26-8.19 (m, 1H), 8.03 (d, J = 8.4 Hz, 1H), 7.62 (dd, J = 8.4, 1.7 Hz, 1H), 4.75 (br s, 1H), 4.54 (d, J = 6.1 Hz, |
| | | | | | 2H), 2.97 (s, 3H). MS |
| | | | | | 271.16 (M + H). |
| 142 | 5-(2,6- dimethylmorpholine-4- carbonyl)benzo[d] thiazole-2- carbaldehyde | 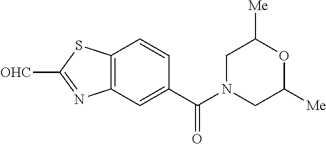 [SMILES: CC1CN(C(=O)c2ccc3sc(C=O)nc3c2)CC(C)O1] | Production Ex. 119 | Production Ex. 24 | MS 305.26 (M + H). |
| 143 | 6-(2,6- dimethylmorpholine-4- carbonyl)benzo[d] thiazole-2- carbaldehyde | 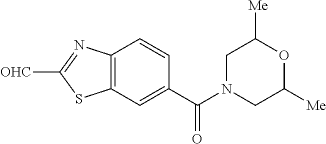 [SMILES: CC1CN(C(=O)c2ccc3nc(C=O)sc3c2)CC(C)O1] | Production Ex. 119 | Production Ex. 25 | MS 323.26 (M + H2O+H). |
| 144 | 6-(2,6- dimethylmorpholine-4- carbonyl)quinoline- 2-carbaldehyde | 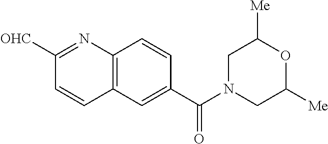 [SMILES: CC1CN(C(=O)c2ccc3nc(C=O)ccc3c2)CC(C)O1] | Production Ex. 119 | Production Ex. 26 | MS 299.33 (M + H). |
| 145 | N-((2- formylbenzo[d] thiazol-6- yl)methyl)methane sulfonamide | 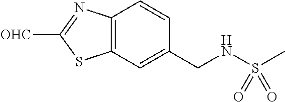 [SMILES: CS(=O)(=O)NCc1ccc2nc(C=O)sc2c1] | Production Ex. 119 | Production Ex. 80 | 1H NMR (270 MHz) δ 10.17 (s, 1H), 8.25 (d, J = 8.5 Hz, 1H), 8.04 (d, J = 1.8 Hz, 1H), 7.61 (dd, J = 8.6, 1.7 Hz, 1H), 4.76 (br s, 1H), 4.53 (d, J = 6.2 Hz, 2H), 2.96 (s, 3H). MS |
| | | | | | 271.17 (M + H). |
| 146 | N-((2- formylquinolin-6- yl)methyl)-N- (tetrahydro-2H- pyran-4- yl)acetamide | 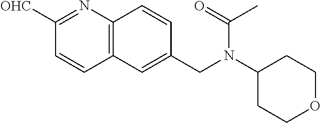 [SMILES: CC(=O)N(Cc1ccc2nc(C=O)ccc2c1)C1CCOCC1] | Production Ex. 119 | Production Ex. 49 | 1H NMR (270 MHz) δ 10.23, 10.22 (each s, combined 1H), 8.33-8.15 (m, 2H), 8.11-7.98 (m, 1H), 7.76-7.63 (m, 2H), 5.02-4.84 (m, 1H), 4.81, 4.75 (each s, combined 2H), 4.06-3.88 (m, 2H), |
| | | | | | 3.57-3.33 (m, 2H), 2.36, |
| | | | | | 2.12 (each s, combined |
| | | | | | 3H), 1.95-1.55 (m, |
| | | | | | 4H). MS 313.33 (M + H). |
| 147 | N-((2- formylquinolin-6- yl)methyl)-N- (tetrahydro-2H- pyran-4-yl) methanesulfonamide | 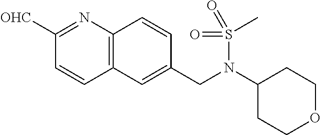 | Production Ex. 119 | Production Ex. 60 | 1H NMR (270 MHz) δ 10.23 (d, J = 0.9 Hz, 1H), 8.37-8.20 (m, 2H), 8.06 (d, J = 8.3 Hz, 1H), 7.96- 7.84 (m, 2H), 4.64 (s, 2H), 4.17-3.89 (m, 3H), 3.49- 3.32 (m, 2H), 2.94 (s, 3H), 1.89-1.64 (m, 4H). MS |
| | | | | | 349.29 (M + H). |
| 148 | (E)-6-(3- morpholino-3- oxoprop-1-en-1- yl)benzo[d]thiazole- 2-carbaldehyde | 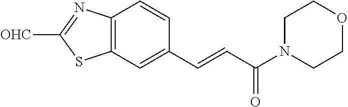 [SMILES: O=Cc1nc2ccc(/C=C/C(=O)N3CCOCC3)cc2s1] | Production Ex. 119 | Production Ex. 27 | 1H NMR (500 MHz) 8 10.17 (s, 1H), 8.23 (d, J = 8.7 Hz, 1H), 8.13 (d, J = 1.6 Hz, 1H), 7.83 (d, J = 15.4 Hz, 1H), 7.79 (dd, J = 8.7, 1.8 Hz, 1H), 7.00 (d, J = 15.4 Hz, 1H), 3.84-3.62 |
| | | | | | (m, 8H). MS 303.11 |
| | | | | | (M + H). |
| 149 | (E)-6-(3- morpholino-3- oxoprop-1-en-1- yl)quinoline-2- carbaldehyde | 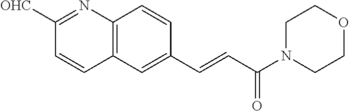 [SMILES: O=Cc1ccc2cc(/C=C/C(=O)N3CCOCC3)ccc2n1] | Production Ex. 119 | Production Ex. 18 | 1H NMR (500 MHz) δ 10.23 (d, J = 0.8 Hz, 1H), 8.33 (d, J = 8.5 Hz, 1H), 8.24 (d, J = 8.8 Hz, 1H), 8.06 (d, J = 8.4 Hz, 1H), 8.02 (dd, J = 8.9, 2.0 Hz, 1H), 7.97 (d, J = 2.0 Hz, |
| | | | | | 1H), 7.89 (d, J = 15.4 Hz, |
| | | | | | 1H), 7.06 (d, J = 15.4 Hz, |
| | | | | | 1H), 3.81-3.69 (m, |
| | | | | | 8H). MS 297.18 (M + H). |
| 150 | N-((2- formylquinolin-6- yl)methyl)-2- morpholinoacetamide | 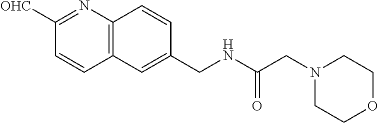 [SMILES: O=Cc1ccc2cc(CNC(=O)CN3CCOCC3)ccc2n1] | Production Ex. 119 | Production Ex. 45 | 1H NMR (270 MHz) δ 10.23 (d, J = 0.9 Hz, 1H), 8.29 (d, J = 8.5 Hz, 1H), 8.24 (d, J = 8.7 Hz, 1H), 8.05 (d, J = 8.3 Hz, 1H), 7.83-7.72 (m, 2H), 4.72 (d, J = 6.2 Hz, 2H), 3.76- 3.66 (m, 4H), 3.14 (s, 2H), |
| | | | | | 2.62-2.52 (m, 4H). MS |
| | | | | | 314.30 (M + H). |
| 151 | 4-azido-6- (morpholine-4- carbonyl)quinoline- 2-carbaldehyde | 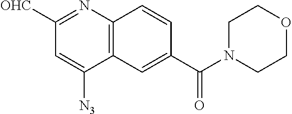 [SMILES: [N-]=[N+]=Nc1cc(C=O)nc2ccc(C(=O)N3CCOCC3)cc12] | Production Ex. 119 | Production Ex. 17 | 1H NMR (270 MHz) δ 10.18 (s, 1H), 8.27 (d, J = 8.8 Hz, 1H), 8.20 (d, J = 1.8 Hz, 1H), 7.87 (dd, J = 8.7, 1.9 Hz, 1H), 7.82 (s, 1H), 3.95-3.38 (m, 8H). MS 330.22 (M + H₂O + H). |
| 152 | 4-((3H- [1,2,3]triazolo [4,5- b]pyridin-3- yl)oxy)-6- (morpholine-4- carbonyl)quinoline- 2-carbaldehyde | 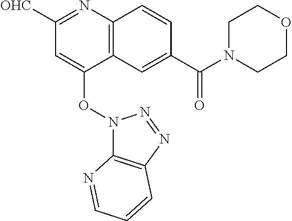 [SMILES: O=Cc1cc(On2nnc3cccnc32)c2cc(C(=O)N3CCOCC3)ccc2n1] | Production Ex. 119 | Production Ex. 33 | MS 405.2 (M + H). |
| 153 | 4-chloro-6- (morpholine-4- carbonyl)quinoline- 2-carbaldehyde | 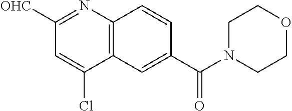 [SMILES: O=Cc1cc(Cl)c2cc(C(=O)N3CCOCC3)ccc2n1] | Production Ex. 119 | Production Ex. 34 | 1H NMR (270 MHz) δ 10.19 (s, 1H), 8.37 (d, J = 1.8 Hz, 1H), 8.34 (d, J = 8.8 Hz, 1H), 8.15 (s, 1H), 7.89 (dd, J = 8.6, 1.8 Hz, 1H), 3.98-3.42 (m, 8H). MS 305.18 (M + H). |
| 154 | 6-(morpholine-4- carbonyl)-4- phenylquinoline-2- carbaldehyde | 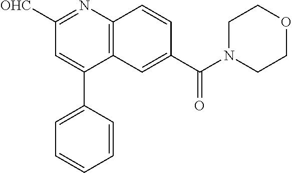 [SMILES: O=Cc1cc(-c2ccccc2)c2cc(C(=O)N3CCOCC3)ccc2n1] | Production Ex. 119 | Production Ex. 95 | 1H NMR (270 MHz) δ 10.28 (s, 1H), 8.38 (d, J = 8.6 Hz, 1H), 8.10-8.00 (m, 2H), 7.84 (dd, J = 8.6, 1.8 Hz, 1H), 7.62-7.45 (m, 5H), 3.78 (s, 8H). MS 347.24 (M + H). |
| 155 | 4-butoxy-6- (morpholine-4- carbonyl)quinoline- 2-carbaldehyde | 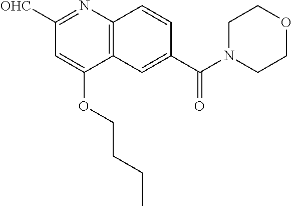 [SMILES: CCCCOc1cc(C=O)nc2ccc(C(=O)N3CCOCC3)cc12] | Production Ex. 119 | Production Ex. 96 | 1H NMR (270 MHz) δ 10.15 (s, 1H), 8.35 (d, J = 2.0 Hz, 1H), 8.21 (d, J = 8.6 Hz, 1H), 7.79 (dd, J = 8.8, 2.0 Hz, 1H), 7.40 (s, 1H), 4.30 (t, J = 6.4 Hz, 2H), 3.97-3.31 (m, 8H), 2.04-1.88 (m, 2H), 1.68- 1.51 (m, 2H), 1.04 (t, J = 7.4 Hz, 3H). MS 343.19 (M + H). |
| 156 | 6-(morpholine-4- carbonyl)-4- (pyridin-3- yl)quinoline-2- carbaldehyde | 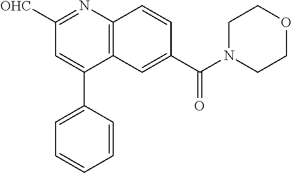 [SMILES: O=Cc1cc(-c2ccccc2)c2cc(C(=O)N3CCOCC3)ccc2n1] | Production Ex. 119 | Production Ex. 97 | 1H NMR (270 MHz) δ 10.28 (s, 1H), 8.82 (dd, J = 4.9, 1.7 Hz, 1H), 8.80- 8.77 (m, 1H), 8.41 (d, J = 8.6 Hz, 1H), 8.04 (s, 1H), 7.97 (d, J = 1.7 Hz, 1H), 7.91-7.84 (m, 2H), 7.55 (dd, J = 7.8, 4.8 Hz, 1H), 3.94-3.30 (m, 8H). MS 348.37 (M + H). |
| 157 | 2-formyl-6- (morpholine-4- carbonyl)quinoline- 4-carbonitrile | 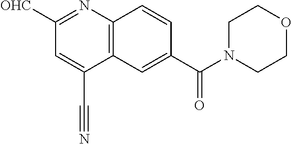 [SMILES: N#Cc1cc(C=O)nc2ccc(C(=O)N3CCOCC3)cc12] | Production Ex. 119 | Production Ex. 88 | 1H NMR (270 MHz) δ 10.23 (s, 1H), 8.44 (d, J = 8.7 Hz, 1H), 8.39 (s, 1H), 8.32 (d, J = 1.8 Hz, 1H), 7.99 (dd, J = 8.7, 1.8 Hz, 1H), 3.99-3.40 (m, 8H). MS 296.13 (M + H). |
| 158 | 6-(3-morpholino-3- oxopropyl)quinoline- 2-carbaldehyde | 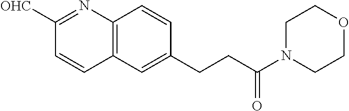 [SMILES: O=Cc1ccc2cc(CCC(=O)N3CCOCC3)ccc2n1] | Production Ex. 119 | Production Ex. 7 | 1H NMR (270 MHz) δ 10.22 (d, J = 0.9 Hz, 1H), 8.26 (d, J = 8.5 Hz, 1H), 8.19 (d, J = 8.6 Hz, 1H), 8.03 (d, J = 8.5 Hz, 1H), 7.77-7.67 (m, 2H), 3.70- 3.54 (m, 6H), 3.47-3.37 |
| | | | | | (m, 2H), 3.29-3.17 (m, |
| | | | | | 2H), 2.81-2.69 (m, |
| | | | | | 2H). MS 299.33 (M + H). |
| 159 | 1-(2-formyl-6- (morpholine-4- carbonyl)quinolin- 4-yl)piperidine-4- carbonitrile | 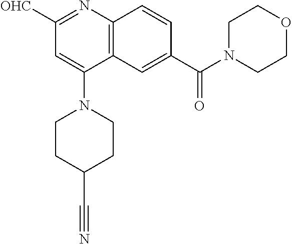 [SMILES: N#CC1CCN(c2cc(C=O)nc3ccc(C(=O)N4CCOCC4)cc23)CC1] | Production Ex. 119 | Production Ex. 109 | 1H NMR (270 MHz) δ 10.16 (s, 1H), 8.25 (d, J = 8.6 Hz, 1H), 8.06 (d, J = 1.9 Hz, 1H), 7.76 (dd, J = 8.7, 1.8 Hz, 1H), 7.54 (s, 1H), 4.02-3.40 (m, 10H), 3.31-3.16 (m, 2H), 3.03- 2.90 (m, 1H), 2.33-2.08 (m, 4H). MS 379.37 (M + H). |
| 160 | 4-([1,1′-biphenyl]- 3-yl)-6- (morpholine-4- carbonyl)quinoline- 2-carbaldehyde | 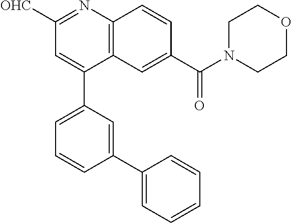 [SMILES: O=Cc1cc(-c2cccc(-c3ccccc3)c2)c2cc(C(=O)N3CCOCC3)ccc2n1] | Production Ex. 119 | Production Ex. 99 | MS 423.33 (M + H). |
| 161 | 4-([1,1′-biphenyl]- 2-yl)-6- (morpholine-4- carbonyl)quinoline- 2-carbaldehyde | 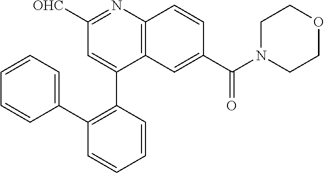 [SMILES: O=Cc1cc(-c2ccccc2-c2ccccc2)c2cc(C(=O)N3CCOCC3)ccc2n1] | Production Ex. 119 | Production Ex. 98 | MS 423.34 (M + H). |
| 162 | 6-(morpholine-4- carbonyl)-[4,8′- biquinoline]-2- carbaldehyde | 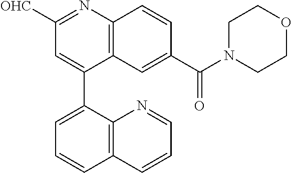 [SMILES: O=Cc1cc(-c2cccc3cccnc23)c2cc(C(=O)N3CCOCC3)ccc2n1] | Production Ex. 119 | Production Ex. 100 | MS 398.35 (M + H). |
| 163 | 6-(morpholine-4- carbonyl)-4-(4- phenoxyphenyl) quinoline-2- carbaldehyde | 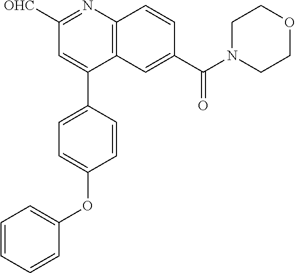 [SMILES: O=Cc1cc(-c2ccc(Oc3ccccc3)cc2)c2cc(C(=O)N3CCOCC3)ccc2n1] | Production Ex. 119 | Production Ex. 101 | MS 439.34 (M + H). |
| 164 | tert-butyl 2- formylquinoline-6- carboxylate | 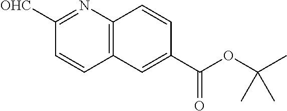 [SMILES: CC(C)(C)OC(=O)c1ccc2nc(C=O)ccc2c1] | Production Ex. 119 | Production Ex. 85 | 1H NMR (270 MHz) δ 10.25 (d, J = 0.9 Hz, 1H), 8.59 (d, J = 1.9 Hz, 1H), 8.43 (d, J = 8.4 Hz, 1H), 8.37 (dd, J = 8.9, 1.9 Hz, 1H), 8.28 (d, J = 8.9 Hz, 1H), 8.09 (d, J = 8.3 Hz, |
| | | | | | 1H), 1.67 (s, 9H). MS |
| | | | | | 258.28 (M + H). |
| 165 | tert-butyl ((2- formylquinolin-6- yl)methyl) (tetrahydro- 2H-pyran-4- yl)carbamate | 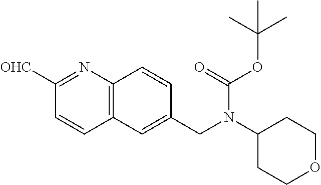 [SMILES: CC(C)(C)OC(=O)N(Cc1ccc2nc(C=O)ccc2c1)C1CCOCC1] | Production Ex. 119 | Production Ex. 112 | 1H NMR (270 MHz) δ 10.22 (s, 1H), 8.30-8.16 (m, 2H), 8.04 (d, J = 8.5 Hz, 1H), 7.77-7.68 (m, 2H), 4.71-4.20 (m, 3H), 4.02-3.90 (m, 2H), 3.51- 3.29 (m, 2H), 1.84-1.21 (m, 13H). MS 371.46 (M + H). |
| 166 | 6-(2-((tetrahydro- 2H-pyran-2- yl)oxy)ethoxy) quinoline-2- carbaldehyde | 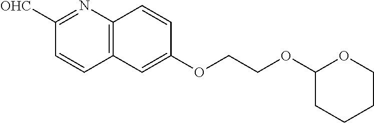 [SMILES: O=Cc1ccc2cc(OCCOC3CCCCO3)ccc2n1] | Production Ex. 119 | Production Ex. 13 | 1H NMR (270 MHz) δ 10.19 (d, J = 0.9 Hz, 1H), 8.20-8.11 (m, 2H), 8.00 (d, J = 8.6 Hz, 1H), 7.52 (dd, J = 9.3, 2.7 Hz, 1H), 7.18 (d, J = 2.7 Hz, 1H), 4.78-4.71 (m, 1H), 4.37- 4.29 (m, 2H), 4.21-4.10 |
| | | | | | (m, 1H), 3.98-3.86 (m, |
| | | | | | 2H), 3.61-3.50 (m, 1H), |
| | | | | | 1.84-1.48 (m, 6H). MS |
| | | | | | 302.33 (M + H). |
| 167 | tert-butyl ((2- formylbenzo[d] thiazol-5- yl)methyl) (tetrahydro- 2H-pyran-4- yl)carbamate | 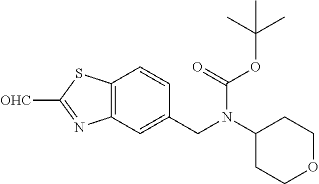 [SMILES: CC(C)(C)OC(=O)N(Cc1ccc2sc(C=O)nc2c1)C1CCOCC1] | Production Ex. 119 | Production Ex. 113 | MS 377.34 (M + H). |
| 168 | tert-butyl 1-(2- formylquinoline-6- carbonyl)piperidine- 4-carboxylate | 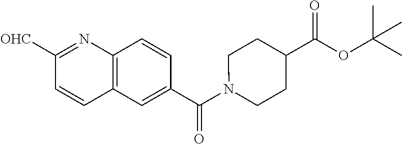 [SMILES: CC(C)(C)OC(=O)C1CCN(C(=O)c2ccc3nc(C=O)ccc3c2)CC1] | Production Ex. 119 | Production Ex. 35 | MS 369.32 (M + H). |
| 169 | tert-butyl (1,1- dioxidotetrahydro- 2H-thiopyran-4- yl)((2- formylquinolin-6- yl)methyl)carbamate | 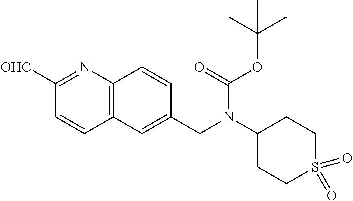 [SMILES: CC(C)(C)OC(=O)N(Cc1ccc2nc(C=O)ccc2c1)C1CCS(=O)(=O)CC1] | Production Ex. 119 | Production Ex. 114 | 1H NMR (270 MHz) δ 10.23 (d, J = 0.9 Hz, 1H), 8.30-8.20 (m, 2H), 8.06 (d, J = 8.5 Hz, 1H), 7.74- 7.67 (m, 2H), 4.64 (s, 2H), 3.17-2.95 (m, 5H), 2.50- 1.92 (m, 4H), 1.44 (br s, 9H). MS 419.24 (M + H). |
| 170 | tert-butyl 4-(2- formylquinoline-6- carbonyl)piperazine- 1-carboxylate | 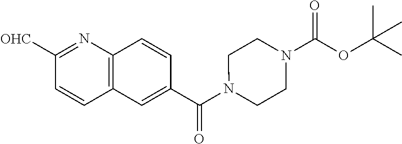 [SMILES: CC(C)(C)OC(=O)N1CCN(C(=O)c2ccc3nc(C=O)ccc3c2)CC1] | Production Ex. 119 | Production Ex. 21 | 1H NMR (270 MHz) δ 10.25 (d, J = 0.9 Hz, 1H), 8.36 (d, J = 8.5 Hz, 1H), 8.32 (d, J = 8.6 Hz, 1H), 8.10 (d, J = 8.5 Hz, 1H), 7.99 (d, J = 1.9 Hz, 1H), 7.82 (dd, J = 8.7, 1.8 Hz, 1H), 3.91-3.33 (m, 8H), 1.48 (s, 9H). MS 370.30 |
| | | | | | (M + H). |
| 171 | tert-butyl ((2- formylquinolin-6- yl)methyl)(1- (oxetan-3- yl)piperidin-4- yl)carbamate | 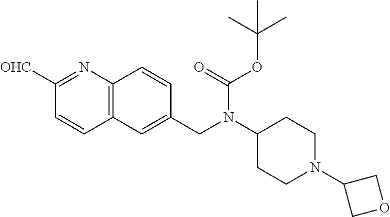 [SMILES: CC(C)(C)OC(=O)N(Cc1ccc2nc(C=O)ccc2c1)C1CCN(C2COC2)CC1] | Production Ex. 119 | Production Ex. 115 | 1H NMR (270 MHz) δ 10.23 (d, J = 1.0 Hz, 1H), 8.29-8.16 (m, 2H), 8.03 (d, J = 8.3 Hz, 1H), 7.76- 7.68 (m, 2H), 4.70-4.53 (m, 6H), 3.53-3.42 (m, 1H), 2.87-2.76 (m, 1H), 2.01-1.29 (m, 17H). MS 426.53 (M + H). |
| 172 | tert-butyl ((2- formylbenzo[d] thiazol-6- yl)methyl) (tetrahydro- 2H-pyran-4- yl)carbamate | 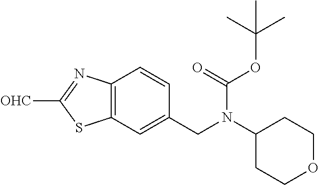 | Production Ex. 119 | Production Ex. 116 | MS 395.41 (M + H₂O + H). |
| 173 | tert-butyl ((2SR,6RS)-2,6- dimethyltetrahydro- 2H-pyran-4- yl)((2- formylquinolin-6- yl)methyl)carbamate | 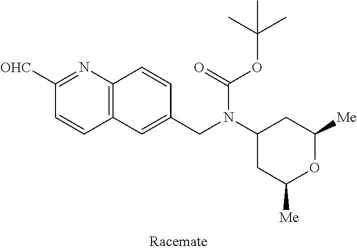 [SMILES: C[C@@H]1CC(N(Cc2ccc3nc(C=O)ccc3c2)C(=O)OC(C)(C)C)C[C@H](C)O1] | Production Ex. 119 | Production Ex. 117 | 1H NMR (270 MHz) δ 10.23 (s, 1H), 8.30-8.17 (m, 2H), 8.04 (d, J = 8.5 Hz, 1H), 7.77-7.65 (m, 2H), 4.67-4.29 (m, 3H), 3.63-3.41 (m, 2H), 1.80- 1.20 (m, 13H), 1.16 (d, J = 6.2 Hz, 6H). MS 399.37 (M + H). |
| 174 | tert-butyl ((2SR,6SR)-2,6- dimethyltetrahydro- 2H-pyran-4- yl)((2- formylquinolin-6- yl)methyl)carbamate | 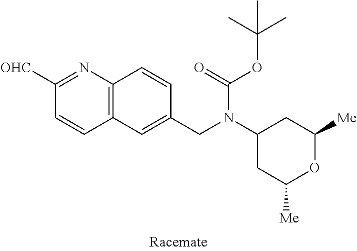 [SMILES: C[C@@H]1CC(N(Cc2ccc3nc(C=O)ccc3c2)C(=O)OC(C)(C)C)C[C@@H](C)O1] | Production Ex. 119 | Production Ex. 118 | 1H NMR (270 MHz) δ 10.23 (d, J = 0.9 Hz, 1H), 8.29-8.19 (m, 2H), 8.05 (d, J = 8.3 Hz, 1H), 7.69- 7.60 (m, 2H), 4.82 (s, 2H), 4.46-4.38 (m, 1H), 3.81- 3.67 (m, 2H), 1.95-1.83 (m, 2H), 1.63-1.46 (m, 2H), 1.40 (s, 9H), 1.13 (d, J = 6.2 Hz, 6H). MS 399.39 (M + H). |
| 175 | 6′-(morpholine-4- carbonyl)-[3,4′- biquinoline]-2′- carbaldehyde | 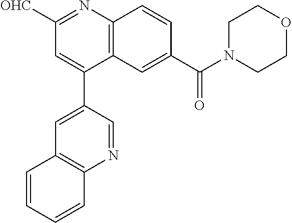 [SMILES: O=Cc1cc(-c2cnc3ccccc3c2)c2cc(C(=O)N3CCOCC3)ccc2n1] | Production Ex. 119 | Production Ex. 104 | 1H NMR (500 MHz) δ 10.29 (s, 1H), 9.04 (d, J = 2.3 Hz, 1H), 8.44-8.40 (m, 1H), 8.36-8.33 (m, 1H), 8.25-8.22 (m, 1H), 8.13 (s, 1H), 8.01-7.99 (m, 1H), 7.96-7.92 (m, 1H), 7.89-7.84 (m, 2H), 7.71-7.67 (m, 1H), 3.82- 3.33 (m, 8H). MS 398.37 (M + H). |
| 176 | 6-(morpholine-4- carbonyl)-4-(2- phenylpyridin-3- yl)quinoline-2- carbaldehyde | 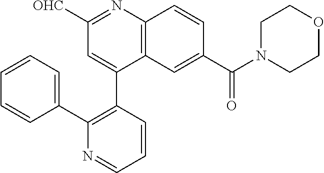 [SMILES: O=Cc1cc(-c2cccnc2-c2ccccc2)c2cc(C(=O)N3CCOCC3)ccc2n1] | Production Ex. 119 | Production Ex. 105 | 1H NMR (500 MHz) δ 10.23 (s, 1H), 8.90 (dd, J = 4.8, 1.7 Hz, 1H), 8.26 (dd, J = 8.6, 0.6 Hz, 1H), 8.00 (s, 1H), 7.73 (dd, J = 7.7, 1.7 Hz, 1H), 7.68 (dd, J = 8.6, 1.8 Hz, 1H), 7.54 (dd, J = 1.9, 0.7 Hz, 1H), 7.47 (dd, J = 7.7, 4.8 Hz, 1H), 7.30-7.20 (m, 2H), 7.11- 7.02 (m, 3H), 3.94-3.00 |
| | | | | | (m, 8H). MS 424.27 |
| | | | | | (M + H). |
| 177 | tert-butyl 4-([1,1′- biphenyl]-2-yl)-2- formylquinoline-6- carboxylate | 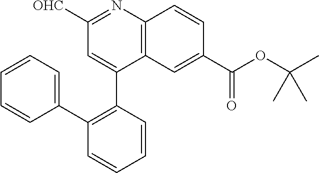 [SMILES: CC(C)(C)OC(=O)c1ccc2nc(C=O)cc(-c3ccccc3-c3ccccc3)c2c1] | Production Ex. 119 | Production Ex. 86 | 1H NMR (500 MHz) δ 10.18 (s, 1H), 8.31-8.30 (m, 1H), 8.18-8.16 (m, 2H), 7.90 (s, 1H), 7.62- 7.50 (m, 3H), 7.42 (dd, J = 7.6, 1.4 Hz, 1H), 7.03- 6.96 (m, 5H), 1.59 (s, 9H). MS 410.29 (M + H). |
| 178 | 4-([1,1′-biphenyl]- 2-yl)-6-(4-(oxetan- 3-yl)piperazine-1- carbonyl)quinoline- 2-carbaldehyde | 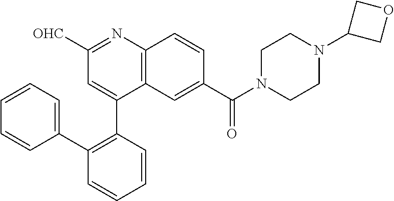 [SMILES: O=Cc1cc(-c2ccccc2-c2ccccc2)c2cc(C(=O)N3CCN(C4COC4)CC3)ccc2n1] | Production Ex. 119 | Production Ex. 74 | 1H NMR (500 MHz) δ 10.18 (s, 1H), 8.20 (dd, J = 8.5, 0.7 Hz, 1H), 7.89 (s, 1H), 7.69-7.63 (m, 2H), 7.62-7.53 (m, 2H), 7.52- 7.47 (m, 1H), 7.38-7.34 (m, 1H), 7.04-6.96 (m, 5H), 4.70-4.65 (m, 2H), 4.61 (t, J = 6.2 Hz, 2H), 3.99-3.61 (m, 2H), 3.55- 3.47 (m, 1H), 3.38-3.18 (m, 2H), 2.53-2.09 (m, 4H). MS 478.34 (M + H) |
| 179 | 4-([1,1′-biphenyl]- 2-yl)-6-(4-(2- hydroxy-3- methoxypropyl)pip erazine-1- carbonyl)quinoline- 2-carbaldehyde | 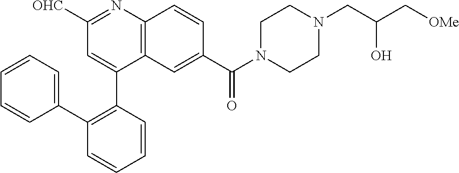 [SMILES: COCC(O)CN1CCN(C(=O)c2ccc3nc(C=O)cc(-c4ccccc4-c4ccccc4)c3c2)CC1] | Production Ex. 119 | Production Ex. 82 | MS 510.37 (M + H) |
| 180 | 4-([1,1′-biphenyl]- 2-yl)-2-formyl-N- (1-(oxetan-3- yl)piperidin-4- yl)quinoline-6- carboxamide | 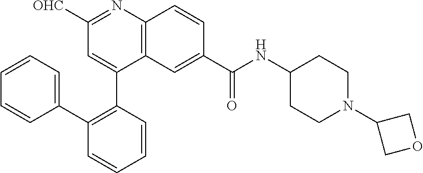 [SMILES: O=Cc1cc(-c2ccccc2-c2ccccc2)c2cc(C(=O)NC3CCN(C4COC4)CC3)ccc2n1] | Production Ex. 119 | Production Ex. 30 | 1H NMR (500 MHz) δ 10.17 (s, 1H), 8.17 (dd, J = 8.7, 0.6 Hz, 1H), 8.07 (dd, J = 8.7, 2.0 Hz, 1H), 8.03 (d, J = 1.9 Hz, 1H), 7.92 (s, 1H), 7.64-7.49 (m, 3H), 7.42 (dd, J = 7.6, 1.4 Hz, 1H), 7.06-6.90 (m, 5H), 5.53 (s, 1H), 4.80-4.69 (m, 2H), 4.68-4.55 (m, 2H), 4.12-4.01 (m, 1H), |
| | | | | | 3.66-3.58 (m, 1H), 3.00- |
| | | | | | 2.90 (m, 2H), 2.21-2.10 |
| | | | | | (m, 2H), 2.09-1.96 (m, |
| | | | | | 4H). MS 492.35 |
| 181 | N-(2-formyl-6- (morpholine-4- carbonyl)quinolin- 4-yl)acetamide | 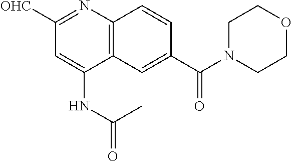 [SMILES: CC(=O)Nc1cc(C=O)nc2ccc(C(=O)N3CCOCC3)cc12] | | Production Ex. 50 | 1H NMR (270 MHz) δ 10.16 (s, 1H), 8.75-8.59 (m, 2H), 8.12 (d, J = 8.6 Hz, 1H), 8.07 (d, J = 1.7 Hz, 1H), 7.66 (dd, J = 8.6, 1.7 Hz, 1H), 3.96-3.39 (m, 8H), 2.38 (s, 3H). MS 327.9 (M + H). |
| 182 | 2-formyl-6- (morpholine-4- carbonyl)quinoline- 4-carboxamide | 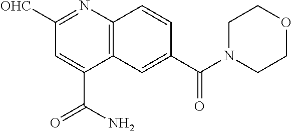 [SMILES: NC(=O)c1cc(C=O)nc2ccc(C(=O)N3CCOCC3)cc12] | | Production Ex. 32 | MS 314.10 (M + H). |
| 183 | (E)-6-(3-(4- (oxetan-3- yl)piperazin-1-yl)- 3-oxoprop-1-en-1- yl)quinoline-2- carbaldehyde | 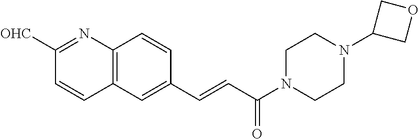 [SMILES: O=Cc1ccc2cc(/C=C/C(=O)N3CCN(C4COC4)CC3)ccc2n1] | Production Ex. 182 | Production Ex. 28 | MS 352.13 (M + H). |
| 184 | (E)-3-(2- formylquinolin-6- yl)-N-(1-(oxetan-3- yl)piperidin-4- yl)acrylamide | 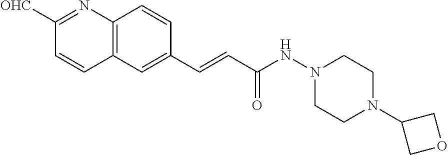 [SMILES: O=Cc1ccc2cc(/C=C/C(=O)NN3CCN(C4COC4)CC3)ccc2n1] | Production Ex. 182 | Production Ex. 29 | MS 366.25 (M + H). |
| 185 | 4-(morpholine-4- carbonyl)thiazole- 2-carbaldehyde | 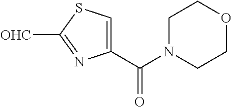 [SMILES: O=Cc1nc(C(=O)N2CCOCC2)cs1] | | CAS No. 1328723- 96-1 (Reagent Supplier 1) | MS 226.84 (M + H). |
| 186 | 5-(morpholine-4- carbonyl) picolinaldehyde | 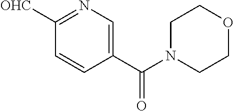 [SMILES: O=Cc1ccc(C(=O)N2CCOCC2)cn1] | Production Ex. 185 | CAS No. 55362-86-2 (Reagent Supplier 1) | 1H NMR (270 MHz) δ 10.10 (s, 1H), 8.85 (dd, J = 4.7, 1.6 Hz, 1H), 7.78- 7.70 (m, 1H), 7.59 (dd, J = 7.7, 4.8 Hz, 1H), 3.92- 3.84 (m, 4H), 3.64-3.54 (m, 2H), 3.19-3.08 (m, |
| | | | | | 2H). MS 221.16 (M + H). |
| 187 | 5-(morpholine-4- carbonyl)thiazole- 2-carbaldehyde | 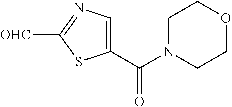 [SMILES: O=Cc1ncc(C(=O)N2CCOCC2)s1] | Production Ex. 185 | CAS No. 79836-80-9 (Reagent Supplier 1) | MS 227.07 (M + H). |
| 188 | 6-(morpholine-4- carbonyl)-2- naphthaldehyde | 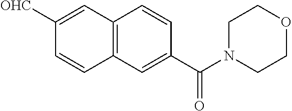 [SMILES: O=Cc1ccc2cc(C(=O)N3CCOCC3)ccc2c1] | | Production Ex. 36 | 1H NMR (270 MHz) δ 10.20 (s, 1H), 8.40-8.36 (m, 1H), 8.11-7.95 (m, 4H), 7.61 (dd, J = 8.5, 1.6 Hz, 1H), 3.94-3.41 (m, 8H). MS 270.26 (M + H). |
| 189 | 4-(4- acetylpiperazin-1- yl)-6-(morpholine-4- carbonyl)quinoline- 2-carbaldehyde | 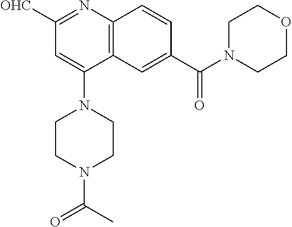 [SMILES: CC(=O)N1CCN(c2cc(C=O)nc3ccc(C(=O)N4CCOCC4)cc23)CC1] | Production Ex. 188 | Production Ex. 111 | 1H NMR (270 MHz) δ 10.16 (s, 1H), 8.27 (d, J = 8.6 Hz, 1H), 8.14 (d, J = 1.8 Hz, 1H), 7.77 (dd, J = 8.6, 1.8 Hz, 1H), 7.52 (s, 1H), 4.04-3.40 (m, 12H), 3.37-3.22 (m, 4H), 2.19 (s, 3H). MS 397.34 (M + H). |
| 190 | 2-(morpholine-4- carbonyl)-1H- pyrrolo [3,2- b]pyridine-5- carbaldehyde | 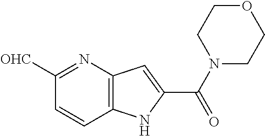 [SMILES: O=Cc1ccc2[nH]c(C(=O)N3CCOCC3)cc2n1] | | Production Ex. 64 | 1H NMR (270 MHz) δ 10.14 (d, J = 0.8 Hz, 1H), 9.75 (br s, 1H), 7.99 (d, J = 8.6 Hz, 1H), 7.87 (dd, J = 8.6, 1.0 Hz, 1H), 7.11 (dd, J = 2.2, 0.9 Hz, 1H), 4.06- 3.89 (m, 4H), 3.87-3.78 (m, 4H). MS 260.15 (M + H). |
| 191 | 2-(morpholine-4- carbonyl)furo[3,2- b]pyridine-5- carbaldehyde | 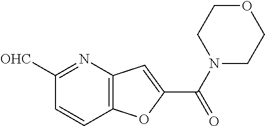 [SMILES: O=Cc1ccc2oc(C(=O)N3CCOCC3)cc2n1] | Production Ex. 190 | Production Ex. 65 | 1H NMR (270 MHz) δ 10.16 (d, J = 0.9 Hz, 1H), 8.10 (d, J = 8.6 Hz, 1H), 8.02-7.94 (m, 1H), 7.55 (d, J = 0.9 Hz, 1H), 3.91- 3.79 (m, 8H). MS 261.21 (M + H). |
| 192 | 5-(morpholine-4- carbonyl)-1H- indole-2- carbaldehyde | 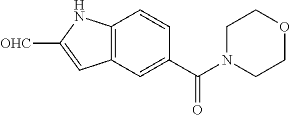 [SMILES: O=Cc1cc2cc(C(=O)N3CCOCC3)ccc2[nH]1] | | Production Ex. 40 | 1H NMR (270 MHz) δ 9.89 (s, 1H), 9.15 (br s, 1H), 7.90-7.83 (m, 1H), 7.51-7.45 (m, 2H), 7.32 (d, J = 2.0 Hz, 1H), 3.86- 3.55 (m, 8H). MS 258.96 (M + H). |
| 193 | 1-methyl-5- (morpholine-4- carbonyl)-1H- indole-2- carbaldehyde | 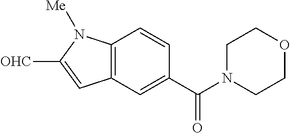 [SMILES: Cn1c(C=O)cc2cc(C(=O)N3CCOCC3)ccc21] | Production Ex. 192 | Production Ex. 47 | 1H NMR (270 MHz) δ 9.92 (s, 1H), 7.88-7.81 (m, 1H), 7.50 (dd, J = 8.8, 1.6 Hz, 1H), 7.44 (d, J = 8.7 Hz, 1H), 7.30 (s, 1H), 4.12 (s, 3H), 3.88-3.54 (m, 8H). MS 273.22 (M + H). |
| 194 | (Z)-1-acetyl-2-((6- (2-((tetrahydro-2H- pyran-2- yl)oxy)ethoxy) quinolin-2- yl)methylene) indolin-3-one | 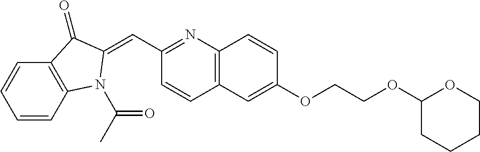 [SMILES: CC(=O)N1/C(=C\c2ccc3cc(OCCOC4CCCCO4)ccc3n2)C(=O)c2ccccc21] | | Production Ex. 166 CAS No. 16800- 68- 3 (Reagent Supplier 3) | 1H NMR (270 MHz) δ 8.21 (d, J = 8.3 Hz, 1H), 8.06 (d, J = 8.5 Hz, 1H), 8.00 (d, J = 9.3 Hz, 1H), 7.83 (dd, J = 7.8, 1.4 Hz, 1H), 7.69-7.59 (m, 1H), 7.55 (d, J = 8.5 Hz, 1H), 7.44 (dd, J = 9.2, 2.7 Hz, 1H), 7.33 (s, 1H), 7.27- 7.18 (m, 1H), 7.10 (d, J = |
| | | | | | 2.7 Hz, 1H), 4.79-4.70 |
| | | | | | (m, 1H), 4.35-4.24 (m, |
| | | | | | 2H), 4.19-4.07 (m, 1H), |
| | | | | | 4.00-3.80 (m, 2H), 3.63- |
| | | | | | 3.48 (m, 1H), 2.13 (s, 3H), |
| | | | | | 1.92-1.47 (m, 6H). MS |
| | | | | | 459.48 (M + H). |
| 195 | 1-acetyl-2-(3- methoxy-4-(2- ((tetrahydro-2H- pyran-2- yl)oxy)ethoxy)ben zylidene )indolin-3- one | 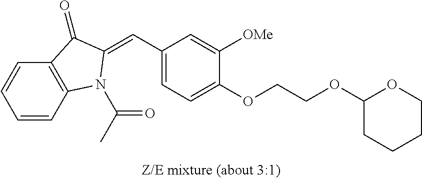 [SMILES: COc1cc(/C=C2/C(=O)c3ccccc3N2C(C)=O)ccc1OCCOC1CCCCO1] | Production Ex. 194 | Production Ex. 12 CAS No. 16800- 68- 3 (Reagent Supplier 3) | 1H NMR (270 MHz) δ 8.29, 8.16 (each d, J = 8.3 Hz, combined 1H), 8.09 (d, J = 2.1 Hz, 0.3H), 7.89- 7.78 (m, 1H), 7.73-7.61 (m, 1H), 7.46-7.37 (m, 0.6H), 7.35-7.21 (m, 1.7H), 7.19-7.12 (m, 0.7H), 7.08 (d, J = 2.0 Hz, 0.7H), 7.04-6.94 (m, 1H), 4.76-4.69 (m, 1H), 4.34- |
| | | | | | 4.23 (m, 2H), 4.19-4.04 |
| | | | | | (m, 1H), 4.02-3.81 (m, |
| | | | | | 5H), 3.59-3.48 (m, 1H), |
| | | | | | 2.64, 2.05 (each s, |
| | | | | | combined 3H), 1.87-1.46 |
| | | | | | (m, 6H). MS 460.23 |
| | | | | | (M + Na). |
| 196 | tert-butyl (Z)-((2- ((1-acetyl-3- oxoindolin-2- ylidene)methyl) quinolin-6- yl)methyl) (tetrahydro- 2H-pyran-4- yl)carbamate | 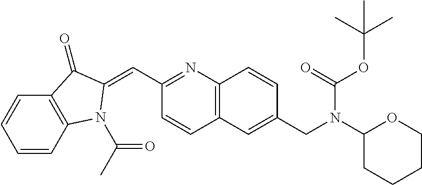 [SMILES: CC(=O)N1/C(=C\c2ccc3cc(CN(C(=O)OC(C)(C)C)C4CCCCO4)ccc3n2)C(=O)c2ccccc21] | Production Ex. 194 | Production Ex. 165 CAS No. 16800- 68- 3 (Reagent Supplier 3) | 1H NMR (270 MHz) δ 8.20 (d, J = 8.3 Hz, 1H), 8.13 (d, J = 8.4 Hz, 1H), 8.06 (d, J = 8.6 Hz, 1H), 7.87-7.80 (m, 1H), 7.69- 7.55 (m, 4H), 7.34 (s, 1H), 7.29-7.20 (m, 1H), 4.67- 4.22 (m, 3H), 4.01-3.89 (m, 2H), 3.50-3.30 (m, 2H), 2.14 (s, 3H), 1.84- 1.20 (m, 13H). MS 528.60 (M + H). |
| 197 | tert-butyl ((2-(((Z)- 1-acetyl-3- oxoindolin-2- ylidene)methyl) quinolin-6- yl)methyl)((2SR, 6RS)-2,6- dimethyltetrahydro- 2H-pyran-4- yl)carbamate | 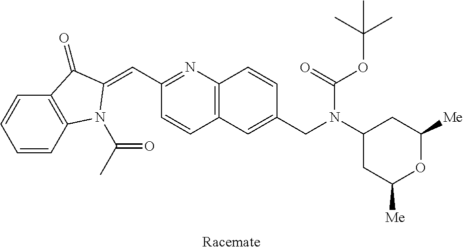 [SMILES: CC(=O)N1/C(=C\c2ccc3cc(CN(C(=O)OC(C)(C)C)C4C[C@@H](C)O[C@@H](C)C4)ccc3n2)C(=O)c2ccccc21] | Production Ex. 194 | Production Ex. 173 CAS No. 16800- 68- 3 (Reagent Supplier 3) | 1H NMR (270 MHz) δ 8.21 (d, J = 8.3 Hz, 1H), 8.14 (d, J = 8.5 Hz, 1H), 8.07 (d, J = 8.6 Hz, 1H), 7.88-7.80 (m, 1H), 7.72- 7.54 (m, 4H), 7.35 (s, 1H), 7.28-7.18 (m, 1H), 4.64- 4.29 (m, 3H), 3.61-3.42 (m, 2H), 2.15 (s, 3H), 1.88- 1.21 (m, 13H), 1.16 (d, J = 6.1 Hz, 6H). MS 556.45 (M + H). |
| 198 | tert-butyl ((2-(((Z)- 1-acetyl-3- oxoindolin-2- ylidene )methyl) quinolin-6- yl)methyl)((2SR, 6SR)-2,6- dimethyltetrahydro- 2H-pyran-4- yl)carbamate | 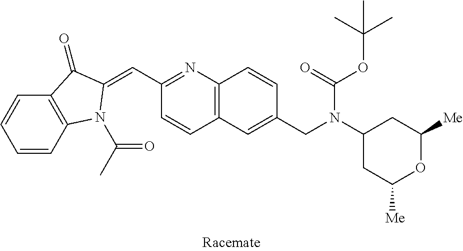 [SMILES: CC(=O)N1/C(=C\c2ccc3cc(CN(C(=O)OC(C)(C)C)C4C[C@@H](C)O[C@H](C)C4)ccc3n2)C(=O)c2ccccc21] | Production Ex. 194 | Production Ex. 174 CAS No. 16800- 68- 3 (Reagent Supplier 3) | 1H NMR (270 MHz) δ 8.21 (d, J = 8.2 Hz, 1H), 8.14 (d, J = 8.4 Hz, 1H), 8.08 (d, J = 8.7 Hz, 1H), 7.87-7.81 (m, 1H), 7.70- 7.51 (m, 4H), 7.35 (s, 1H), 7.29-7.21 (m, 1H), 4.83- 4.76 (m, 2H), 4.48-4.35 (m, 1H), 3.83-3.65 (m, 2H), 2.14 (s, 3H), 1.96- 1.83 (m, 2H), 1.61-1.46 (m, 2H), 1.41 (s, 9H), 1.13 (d, J = 6.2 Hz, 6H). MS 556.47 (M + H). |
| 199 | tert-butyl (Z)-2- ((1-acetyl-3- oxoindolin-2- ylidene )methyl) quinoline-6- carboxylate | 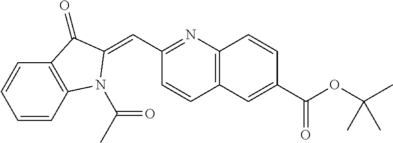 [SMILES: CC(=O)N1/C(=C\c2ccc3cc(C(=O)OC(C)(C)C)ccc3n2)C(=O)c2ccccc21] | | Production Ex. 164 CAS No. 16800- 68- 3 (Reagent Supplier 3) | 1H NMR (270 MHz) δ 8.50 (d, J = 1.9 Hz, 1H), 8.32-8.25 (m, 2H), 8.19 (d, J = 8.3 Hz, 1H), 8.11 (d, J = 8.7 Hz, 1H), 7.87-7.81 (m, 1H), 7.70-7.61 (m, 2H), 7.33 (s, 1H), 7.29- 7.21 (m, 1H), 2.14 (s, 3H), 1.65 (s, 9H). MS 415.41 (M + H). |
| 200 | tert-butyl (Z)-1-(2- ((1-acetyl-3- oxoindolin-2- ylidene)methyl) quinoline-6- carbonyl)piperidine- 4-carboxylate | 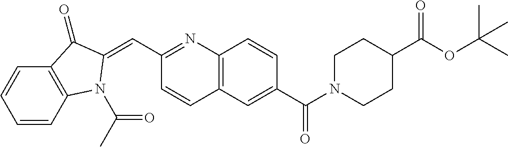 [SMILES: CC(=O)N1/C(=C\c2ccc3cc(C(=O)N4CCC(C(=O)OC(C)(C)C)CC4)ccc3n2)C(=O)c2ccccc21] | Production Ex. 199 | Production Ex. 168 CAS No. 16800- 68- 3 (Reagent Supplier 3) | 1H NMR (270 MHz) δ 8.26-8.16 (m, 2H), 8.13 (d, J = 8.8 Hz, 1H), 7.91- 7.80 (m, 2H), 7.77-7.61 (m, 3H), 7.34 (s, 1H), 7.29- 7.21 (m, 1H), 4.71-4.36 (m, 1H), 3.93-3.58 (m, 1H), 3.24-2.97 (m, 2H), 2.63-2.44 (m, 1H), 2.13 |
| | | | | | (s, 3H), 2.04-1.58 (m, |
| | | | | | 4H), 1.47 (s, 9H). |
| | | | | | MS 526.37 (M + H). |
| 201 | tert-butyl (Z)-((2- ((1-acetyl-3- oxoindolin-2- ylidene)methyl) qui nolin-6- yl)methyl)(1,1- dioxidotetrahydro- 2H-thiopyran-4- yl)carbamate | 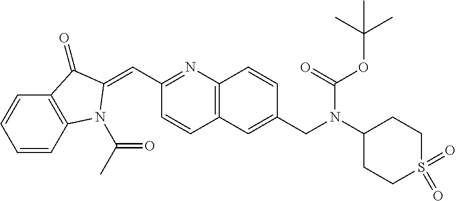 [SMILES: CC(=O)N1/C(=C\c2ccc3cc(CN(C(=O)OC(C)(C)C)C4CCS(=O)(=O)CC4)ccc3n2)C(=O)c2ccccc21] | Production Ex. 199 | Production Ex. 169 CAS No. 16800- 68- 3 (Reagent Supplier 3) | 1H NMR (270 MHz) δ 8.24-8.03 (m, 3H), 7.84 (dd, J = 7.5, 1.4 Hz, 1H), 7.71-7.56 (m, 4H), 7.34 (s, 1H), 7.29-7.20 (m, 1H), 4.68-4.30 (m, 3H), 3.20-2.92 (m, 4H), 2.46- 2.18 (m, 2H), 2.15 (s, 3H), 2.10-1.94 (m, 2H), 1.44 (br s, 9H). MS 576.35 (M + H). |
| 202 | tert-butyl (Z)-4-(2- ((1-acetyl-3- oxoindolin-2- ylidene )methyl) quinoline-6- carbonyl)piperazine- 1-carboxylate | 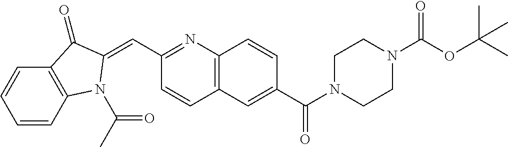 [SMILES: CC(=O)N1/C(=C\c2ccc3cc(C(=O)N4CCN(C(=O)OC(C)(C)C)CC4)ccc3n2)C(=O)c2ccccc21] | Production Ex. 199 | Production Ex. 170 CAS No. 16800- 68- 3 (Reagent Supplier 3) | 1H NMR (270 MHz) δ 8.26-8.11 (m, 3H), 7.90 (d, J = 1.8 Hz, 1H), 7.84 (dd, J = 7.6, 1.5 Hz, 1H), 7.74 (dd, J = 8.6, 1.9 Hz, 1H), 7.70-7.62 (m, 2H), 7.33 (s, 1H), 7.29-7.21 (m, 1H), 3.89-3.34 (m, 8H), 2.14 (s, 3H), 1.48 (s, 9H). MS 527.49 (M + H). |
| 203 | tert-butyl (Z)-((2- ((1-acetyl-3- oxoindolin-2- ylidene )methyl) quinolin-6- yl)methyl)(1- (oxetan-3- yl)piperidin-4- yl)carbamate | 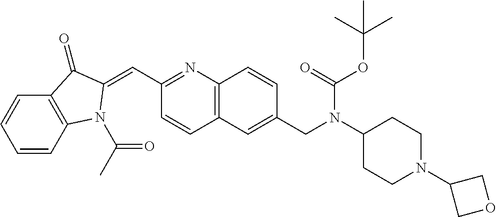 [SMILES: CC(=O)N1/C(=C\c2ccc3cc(CN(C(=O)OC(C)(C)C)C4CCN(C5COC5)CC4)ccc3n2)C(=O)c2ccccc21] | Production Ex. 199 | Production Ex. 171 CAS No. 16800- 68- 3 (Reagent Supplier 3) | 1H NMR (270 MHz) δ 8.20 (d, J = 8.3 Hz, 1H), 8.12 (d, J = 8.5 Hz, 1H), 8.06 (d, J = 8.6 Hz, 1H), 7.84 (dd, J = 7.8, 1.4 Hz, 1H), 7.70-7.55 (m, 4H), 7.34 (s, 1H), 7.28-7.20 (m, 1H), 4.67-4.48 (m, 6H), 4.31-4.12 (m, 1H), 3.49-3.36 (m, 1H), 2.80- 2.69 (m, 2H), 2.14 (s, 3H), 1.96-1.24 (m, 15H). MS 583.61 (M + H). |
| 204 | tert-butyl (Z)-4- ([1,1′-biphenyl]-2- yl)-2-((1-acetyl-3- oxoindolin-2- ylidene )methyl) quinoline-6- carboxylate | 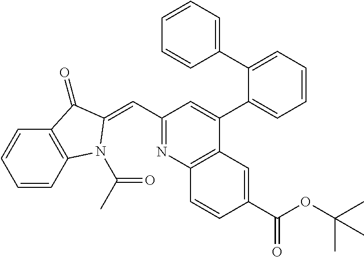 [SMILES: CC(=O)N1/C(=C\c2cc(-c3ccccc3-c3ccccc3)c3cc(C(=O)OC(C)(C)C)ccc3n2)C(=O)c2ccccc21] | Production Ex. 199 | Production Ex. 177 CAS No. 16800- 68- 3 (Reagent Supplier 3) | 1H NMR (500 MHz) δ 8.31 (dd, J = 1.9, 0.6 Hz, 1H), 8.19-8.16 (m, 1H), 8.13 (dd, J = 8.8, 1.9 Hz, 1H), 8.02 (dd, J = 8.9, 0.6 Hz, 1H), 7.82-7.79 (m, 1H), 7.66-7.50 (m, 4H), 7.46-7.43 (m, 1H), 7.35 (s, 1H), 7.24-7.20 (m, 1H), 7.17 (s, 1H), 7.04- 6.96 (m, 5H), 2.00 (s, 3H), 1.58 (s, 9H). MS 567.33 (M + H). |
| 205 | tert-butyl (Z)-((2- ((1-acetyl-3- oxoindolin-2- ylidene)methyl) benzo[d]thiazol-5- yl)methyl)(tetrahydro- 2H-pyran-4- yl)carbamate | 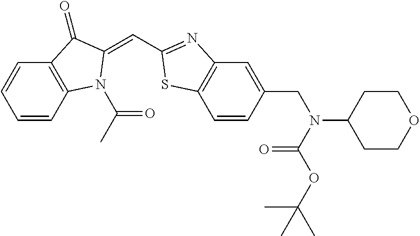 [SMILES: CC(=O)N1/C(=C\c2nc3cc(CN(C(=O)OC(C)(C)C)C4CCOCC4)ccc3s2)C(=O)c2ccccc21] | | Production Ex. 167 CAS No. 16800- 68- 3 (Reagent Supplier 3) | 1H NMR (270 MHz) δ 8.21 (d, J = 8.3 Hz, 1H), 7.97-7.93 (m, 1H), 7.89- 7.81 (m, 2H), 7.72-7.63 (m, 1H), 7.42-7.25 (m, 3H), 4.64-4.30 (m, 3H), 4.00-3.89 (m, 2H), 3.50- 3.28 (m, 2H), 2.24 (s, 3H), 1.83-1.22 (m, 13H). MS 534.39 (M + H). |
| 206 | tert-butyl (E)-((2- ((1-acetyl-3- oxoindolin-2- ylidene )methyl) benzo[d]thiazol-5- yl)methyl)(tetrahydro- 2H-pyran-4- yl)carbamate | 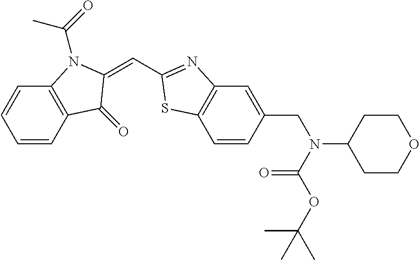 [SMILES: CC(=O)N1/C(=C/c2nc3cc(CN(C(=O)OC(C)(C)C)C4CCOCC4)ccc3s2)C(=O)c2ccccc21] | | Production Ex. 167 CAS No. 16800- 68- 3 (Reagent Supplier 3) | 1H NMR (270 MHz) δ 8.26 (s, 1H), 8.10 (d, J = 8.3 Hz, 1H), 7.97-7.88 (m, 3H), 7.74-7.65 (m, 1H), 7.43-7.29 (m, 2H), 4.65-4.25 (m, 3H), 4.00- 3.89 (m, 2H), 3.50-3.29 (m, 2H), 2.75 (s, 3H), 1.86- 1.29 (m, 13H). MS 534.40 (M + H). |
| 207 | tert-butyl (Z)-((2- ((1-acetyl-3- oxoindolin-2- ylidene )methyl) benzo[d]thiazol-6- yl)methyl)(tetrahydro- 2H-pyran-4- yl)carbamate | 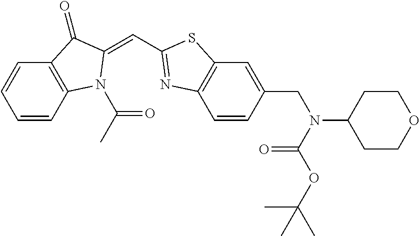 [SMILES: CC(=O)N1/C(=C\c2nc3ccc(CN(C(=O)OC(C)(C)C)C4CCOCC4)cc3s2)C(=O)c2ccccc21] | Production Ex. 205 | Production Ex. 172 CAS No. 16800- 68- 3 (Reagent Supplier 3) | 1H NMR (270 MHz) δ 8.21 (d, J = 8.3 Hz, 1H), 8.04 (d, J = 8.5 Hz, 1H), 7.87-7.80 (m, 1H), 7.78- 7.74 (m, 1H), 7.72-7.63 (m, 1H), 7.45-7.38 (m, 1H), 7.33-7.24 (m, 2H), 4.65-4.20 (m, 3H), 4.01- 3.91 (m, 2H), 3.48-3.33 (m, 2H), 2.26 (s, 3H), 1.84- 1.28 (m, 13H). MS 534.48 (M + H). |
| 208 | tert-butyl (E)-((2- ((1-acetyl-3- oxoindolin-2- ylidene)methyl) benzo[d]thiazol-6- yl)methyl)(tetrahydro- 2H-pyran-4- yl)carbamate | 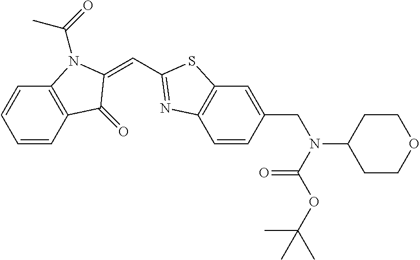 [SMILES: CC(=O)N1/C(=C/c2nc3ccc(CN(C(=O)OC(C)(C)C)C4CCOCC4)cc3s2)C(=O)c2ccccc21] | Production Ex. 206 | Production Ex. 172 CAS No. 16800- 68- 3 (Reagent Supplier 3) | 1H NMR (270 MHz) δ 8.25 (s, 1H), 8.11 (d, J = 8.3 Hz, 1H), 8.03 (d, J = 8.4 Hz, 1H), 7.92 (dd, J = 7.7, 1.5 Hz, 1H), 7.83 (d, J = 1.6 Hz, 1H), 7.74-7.66 (m, 1H), 7.42 (dd, J = 8.5, 1.8 Hz, 1H), 7.37-7.29 (m, 1H), 4.64-4.25 (m, 3H), 4.03-3.90 (m, 2H), 3.49-3.32 (m, 2H), 2.75 (s, 3H), 1.85-1.29 (m, 13H). MS 534.46 (M + H). |
| 209 | 4-([1,1′-biphenyl]- 2-yl)-2- methylquinoline-6- carboxamide | 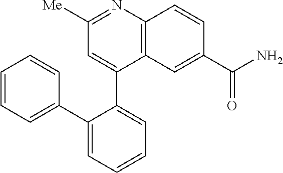 [SMILES: Cc1cc(-c2ccccc2-c2ccccc2)c2cc(C(N)=O)ccc2n1] | | Production Ex. 94 | 1H NMR (500 MHz) δ 8.02-7.99 (m, 2H), 7.94 (dd, J = 8.6, 2.1 Hz, 1H), 7.61-7.57 (m, 2H), 7.54- 7.50 (m, 1H), 7.43-7.41 (m, 1H), 7.17 (s, 1H), 7.09- 7.02 (m, 5H), 6.09-5.38 (m, 2H), 2.69 (s, 3H). MS 339.27 (M + H). |
| 210 | 2-methyl-3- (naphthalen-1- yl)quinoline-6- carboxamide | 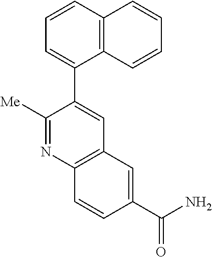 [SMILES: Cc1nc2ccc(C(N)=O)cc2cc1-c1cccc2ccccc12] | Production Ex. 209 | Production Ex. 422 | 1H NMR (500 MHz, dimethylsulfoxide-d6) δ 8.56 (d, J = 2.0 Hz, 1H), 8.30 (s, 1H), 8.26-8.17 (m, 2H), 8.12-8.06 (m, 3H), 7.67 (t, J = 7.6 Hz, 1H), 7.60-7.52 (m, 3H), 7.47 (d, J = 7.8 Hz, 1H), 7.35 (d, J = 8.5 Hz, 1H), 2.34 (s, 3H). MS 313.2 (M + H). |
| 211 | (2-methyl-4- (1,2,3,4- tetrahydroisoquinolin- 6-yl)quinolin-6- yl)(morpholino) methanone | 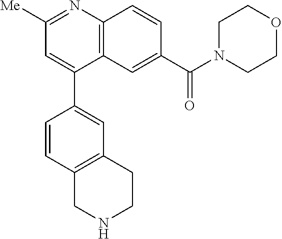 [SMILES: Cc1cc(-c2ccc3c(c2)CCNC3)c2cc(C(=O)N3CCOCC3)ccc2n1] | | Production Ex. 337 | 1H NMR (500 MHz) δ 8.14-8.09 (m, 1H), 7.88 (d, J = 1.9 Hz, 1H), 7.69 (dd, J = 8.6, 1.9 Hz, 1H), 7.35 (dd, J = 7.8, 1.8 Hz, 1H), 7.32-7.20 (m, 3H), 4.40 (s, 2H), 3.77 (s, 10H), 3.18 (t, J = 6.1 Hz, 2H), 2.80 (s, 3H). MS 388.35 (M + H). |
| 212 | N-((4-([1,1′- biphenyl]-2-yl)-2- methylquinolin-6- yl)methyl)tetrahydro- 2H-pyran-4- amine | 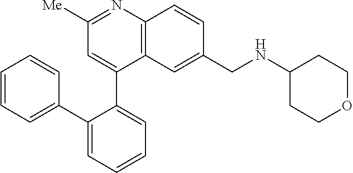 [SMILES: Cc1cc(-c2ccccc2-c2ccccc2)c2cc(CNC3CCOCC3)ccc2n1] | | Production Ex. 400 | 1H NMR (500 MHz) δ 7.93 (d, J = 8.6 Hz, 1H), 7.61-7.54 (m, 3H), 7.53- 7.46 (m, 1H), 7.44 (d, J = 1.9 Hz, 1H), 7.42-7.37 (m, 1H), 7.09-7.00 (m, 6H), 4.00-3.93 (m, 2H), 3.82 (s, 2H), 3.41-3.31 (m, 2H), 2.72-2.60 (m, 4H), 1.85-1.76 (m, 2H), 1.47-1.37 (m, 2H). MS |
| | | | | | 409.31 (M + H). |
| 213 | N-((2-methyl-4-(1- methyl-1H- pyrazol-4- yl)quinolin-6- yl)methyl)tetrahydro- 2H-pyran-4- amine | 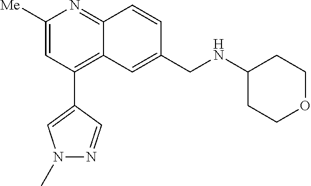 [SMILES: Cc1cc(-c2cnn(C)c2)c2cc(CNC3CCOCC3)ccc2n1] | Production Ex. 212 | Production Ex. 357 | 1H NMR (500 MHz) δ 8.04-8.01 (m, 2H), 7.81 (d, J = 0.7 Hz, 1H), 7.72 (s, 1H), 7.69 (dd, J = 8.6, 1.9 Hz, 1H), 7.23 (s, 1H), 4.05 (s, 3H), 4.01-3.95 (m, 4H), 3.42-3.35 (m, 2H), 2.80-2.72 (m, 4H), 1.91- 1.84 (m, 2H), 1.51-1.42 (m, 2H). MS 337.23 (M + H). |
| 214 | 4-(1H-indazol-4- yl)-2- methylquinoline-6- carboxylic acid | 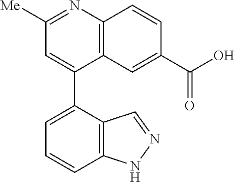 [SMILES: Cc1cc(-c2cccc3[nH]ncc23)c2cc(C(=O)O)ccc2n1] | | Production Ex. 338 | 1H NMR (500 MHz, dimethylsulfoxide-d6) δ 8.35 (d, J = 1.8 Hz, 1H), 8.20 (dd, J = 8.8, 1.9 Hz, 1H), 8.11 (d, J = 8.8 Hz, 1H), 7.75 (d, J = 7.5 Hz, 2H), 7.60 (s, 1H), 7.57 (dd, J = 8.6, 7.0 Hz, 1H), 7.29- 7.24 (m, 1H), 2.77 (s, 3H). MS 304.16 (M + H). |
| 215 | 4-([1,1′-biphenyl]- 3-yl)-2- methylquinoline-6- carboxylic acid | 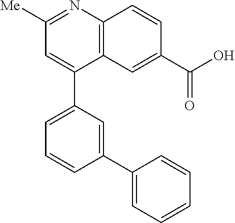 [SMILES: Cc1cc(-c2cccc(-c3ccccc3)c2)c2cc(C(=O)O)ccc2n1] | Production Ex. 214 | Production Ex. 342 | 1H NMR (399 MHz, dimethylsulfoxide-d6) δ 8.55 (d, J = 2.2 Hz, 1H), 8.21 (dd, J = 8.7, 1.9 Hz, 1H), 8.09 (d, J = 8.8 Hz, 1H), 7.91-7.83 (m, 2H), 7.81-7.75 (m, 2H), 7.71 (t, J = 7.6 Hz, 1H), 7.61-7.54 (m, 2H), 7.53-7.46 (m, 2H), 7.44-7.37 (m, 1H), 2.75 (s, 3H). MS 340.14 (M + H). |
| 216 | 2-methyl-4- (naphthalen-1- yl)quinoline-6- carboxylic acid | 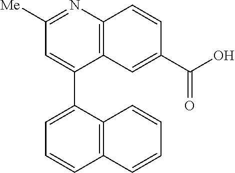 [SMILES: Cc1cc(-c2cccc3ccccc23)c2cc(C(=O)O)ccc2n1] | Production Ex. 214 | Production Ex. 343 | 1H NMR (400 MHz, dimethylsulfoxide-d6) δ 8.20 (dd, J = 8.7, 1.8 Hz, 1H), 8.16-8.02 (m, 3H), 7.87 (d, J = 1.8 Hz, 1H), 7.70 (dd, J = 8.4, 7.0 Hz, 1H), 7.61-7.52 (m, 2H), 7.49 (s, 1H), 7.43-7.36 (m, 1H), 7.24 (d, J = 8.5 Hz, 1H), 2.76 (s, 3H). MS 314.13 (M + H). |
| 217 | 4-([1,1′-biphenyl]- 4-yl)-2- methylquinoline-6- carboxylic acid | 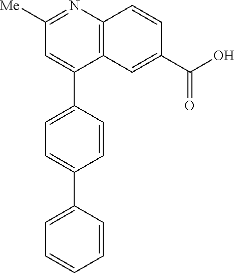 [SMILES: Cc1cc(-c2ccc(-c3ccccc3)cc2)c2cc(C(=O)O)ccc2n1] | Production Ex. 214 | Production Ex. 351 | 1H NMR (400 MHz, dimethylsulfoxide-d6) δ 8.52 (d, J = 2.1 Hz, 1H), 8.22 (dd, J = 8.8, 1.9 Hz, 1H), 8.06 (d, J = 8.7 Hz, 1H), 7.94-7.89 (m, 2H), 7.84-7.78 (m, 2H), 7.67 (d, J = 8.2 Hz, 2H), 7.56- 7.49 (m, 3H), 7.43 (t, J = 7.3 Hz, 1H), 2.74 (s, 3H). MS 340.15 (M + H). |
| 218 | 2-methyl-[4,8′- biquinoline]-6- carboxylic acid | 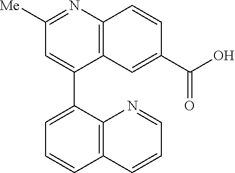 [SMILES: Cc1cc(-c2cccc3cccnc23)c2cc(C(=O)O)ccc2n1] | Production Ex. 214 | Production Ex. 352 | 1H NMR (400 MHz, dimethylsulfoxide-d6) δ 8.73 (q, J = 2.3 Hz, 1H), 8.53 (dd, J = 8.2, 2.1 Hz, 1H), 8.21 (dd, J = 7.1, 2.5 Hz, 1H), 8.17-8.12 (m, 1H), 8.05 (d, J = 8.7 Hz, 1H), 7.86-7.77 (m, 3H), 7.59 (dd, J = 8.3, 4.1 Hz, 1H), 7.48 (s, 1H), 2.74 (s, 3H). MS 315.14 (M + H). |
| 219 | 2-methyl-4- (naphthalen-2- yl)quinoline-6- carboxylic acid | 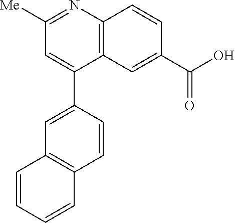 [SMILES: Cc1cc(-c2ccc3ccccc3c2)c2cc(C(=O)O)ccc2n1] | Production Ex. 214 | Production Ex. 353 | 1H NMR (400 MHz, dimethylsulfoxide-d6) δ 8.50 (d, J = 1.9 Hz, 1H), 8.21 (dd, J = 8.8, 2.0 Hz, 1H), 8.18-8.13 (m, 2H), 8.13-8.04 (m, 3H), 7.74- 7.68 (m, 1H), 7.68-7.61 (m, 2H), 7.59 (s, 1H), 2.77 (s, 3H). MS 314.14 (M + H). |
| 220 | (2-methyl-4-(N- tritylindazol-4- yl)quinolin-6- yl)(morpholino) methanone | 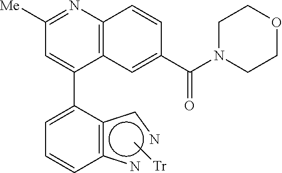 | | Production Ex. 413 | MS 615.47 (M + H). |
| 221 | 4-(3- bromophenyl)-1- trityl-1H-pyrazole | 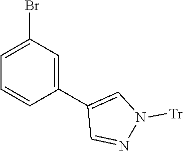 | Production Ex. 220 | CAS No. 916792-28- 4 (Reagent Supplier 5) | 1H NMR (500 MHz) δ 7.92 (d, J = 0.9 Hz, 1H), 7.62 (d, J = 0.9 Hz, 1H), 7.55 (t, J = 1.8 Hz, 1H), 7.36-7.26 (m, 12H), 7.21- 7.16 (m, 6H). MS 465.21 (M + H). |
| 222 | (2-methyl-4-(1- trityl-1H-pyrazol- 4-yl)quinolin-6- yl)(morpholino) methanone | 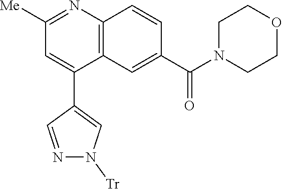 | Production Ex. 220 | Production Ex. 238 | 1H NMR (500 MHz) δ 8.10 (d, J = 1.9 Hz, 1H), 8.06 (dd, J = 8.6, 0.6 Hz, 1H), 7.97 (d, J = 0.8 Hz, 1H), 7.71 (d, J = 0.8 Hz, 1H), 7.68 (dd, J = 8.6, 1.9 Hz, 1H), 7.40-7.34 (m, 9H), 7.30-7.21 (m, 7H), 4.00-3.25 (m, 8H), 2.74 (s, 3H). MS 565.37 (M + H). |
| 223 | ethyl 3-chloro-2- methylquinoline-6- carboxylate | 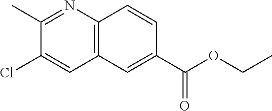 [SMILES: CCOC(=O)c1ccc2nc(C)c(Cl)cc2c1] | | Production Ex. 63 | 1H NMR (500 MHz) δ 8.48 (d, J = 1.9 Hz, 1H), 8.28 (dd, J = 8.8, 1.9 Hz, 1H), 8.20 (s, 1H), 8.04 (d, J = 8.8 Hz, 1H), 4.45 (q, J = 7.1 Hz, 2H), 2.84 (s, 3H), 1.45 (t, J = 7.1 Hz, |
| | | | | | 3H). MS 250.05 (M + H). |
| 224 | tert-butyl 4-([1,1′- biphenyl]-3-yl)-2- formylquinoline-6- carboxylate | 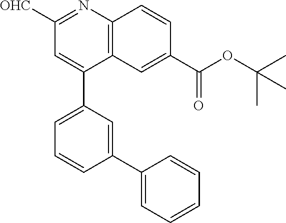 [SMILES: CC(C)(C)OC(=O)c1ccc2nc(C=O)cc(-c3cccc(-c4ccccc4)c3)c2c1] | | Production Ex. 432 | 1H NMR (399 MHz) δ 10.29 (s, 1H), 8.81-8.79 (m, 1H), 8.39-8.33 (m, 2H), 8.09 (s, 1H), 7.79- 7.75 (m, 2H), 7.68-7.62 (m, 3H), 7.56-7.52 (m, 1H), 7.50-7.45 (m, 2H), 7.41-7.36 (m, 1H), 1.58 (s, 9H). MS 410.18 (M + H). |
| 225 | tert-butyl 2-formyl- 4-(naphthalen-1- yl)quinoline-6- carboxylate | 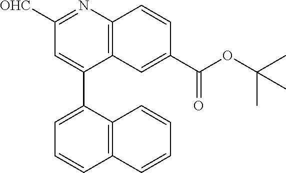 [SMILES: CC(C)(C)OC(=O)c1ccc2nc(C=O)cc(-c3cccc4ccccc34)c2c1] | Production Ex. 224 | Production Ex. 433 | 1H NMR (400 MHz) δ 10.33 (s, 1H), 8.41-8.29 (m, 2H), 8.25 (d, J = 1.8 Hz, 1H), 8.11 (s, 1H), 8.06- 7.96 (m, 2H), 7.63 (dd, J = 8.4, 7.1 Hz, 1H), 7.56- 7.50 (m, 1H), 7.49-7.45 (m, 1H), 7.38-7.33 (m, 1H), 7.32-7.27 (m, 1H), 1.48 (s, 9H). MS 384.13 (M + H). |
| 226 | tert-butyl 4-([1,1′- biphenyl]-4-yl)-2- formylquinoline-6- carboxylate | 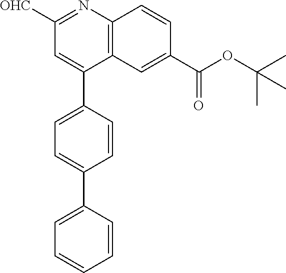 [SMILES: CC(C)(C)OC(=O)c1ccc2nc(C=O)cc(-c3ccc(-c4ccccc4)cc3)c2c1] | Production Ex. 224 | Production Ex. 434 | 1H NMR (400 MHz) δ 10.22 (s, 1H), 8.77-8.74 (m, 1H), 8.31-8.25 (m, 2H), 8.01 (s, 1H), 7.76- 7.70 (m, 2H), 7.65-7.60 (m, 2H), 7.59-7.54 (m, 2H), 7.47-7.41 (m, 2H), 7.38-7.32 (m, 1H), 1.54 (s, 9H). MS 410.20 (M + H). |
| 227 | tert-butyl 2-formyl- [4,8′-biquinoline]- 6-carboxylate | 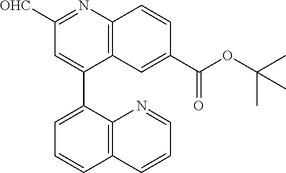 [SMILES: CC(C)(C)OC(=O)c1ccc2nc(C=O)cc(-c3cccc4cccnc34)c2c1] | Production Ex. 224 | Production Ex. 435 | 1H NMR (400 MHz) δ 10.25 (s, 1H), 8.71 (dd, J = 4.2, 1.8 Hz, 1H), 8.28 (d, J = 9.0 Hz, 1H), 8.25-8.20 (m, 2H), 8.15 (d, J = 1.8 Hz, 1H), 8.07 (s, 1H), 7.96 (dd, J = 7.7, 2.2 Hz, 1H), 7.70-7.61 (m, 2H), 7.39 (dd, J = 8.3, 4.1 Hz, 1H), 1.42 (s, 9H). MS 385.17 (M + H) |
| 228 | tert-butyl 2-formyl- 4-(naphthalen-2- yl)quinoline-6- carboxylate | 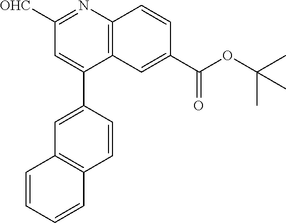 [SMILES: CC(C)(C)OC(=O)c1ccc2nc(C=O)cc(-c3ccc4ccccc4c3)c2c1] | Production Ex. 224 | Production Ex. 436 | 1H NMR (400 MHz) δ 10.31 (s, 1H), 8.80-8.77 (m, 1H), 8.36 (d, J = 1.4 Hz, 2H), 8.13 (s, 1H), 8.07- 8.02 (m, 2H), 7.99-7.92 (m, 2H), 7.66 (dd, J = 8.5, 1.7 Hz, 1H), 7.64-7.56 (m, 2H), 1.57 (s, 9H). MS 384.18 (M + H). |
| 229 | 4-(naphthalen-1- yl)-6-(4-(oxetan-3- yl)piperazine-1- carbonyl)quinoline- 2-carbaldehyde | 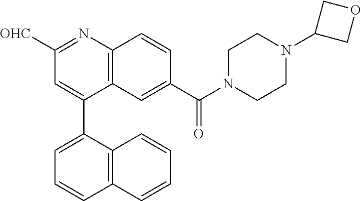 [SMILES: O=Cc1cc(-c2cccc3ccccc23)c2cc(C(=O)N3CCN(C4COC4)CC3)ccc2n1] | Production Ex. 224 | Production Ex. 274 | 1H NMR (400 MHz) δ 10.33 (s, 1H), 8.44-8.38 (m, 1H), 8.12 (s, 1H), 8.04 (d, J = 8.3 Hz, 1H), 7.99 (d, J = 8.2 Hz, 1H), 7.86 (dd, J = 8.7, 1.8 Hz, 1H), 7.67- 7.61 (m, 1H), 7.55-7.42 (m, 3H), 7.35-7.28 (m, 1H), 7.22 (d, J = 8.5 Hz, 1H), 4.67-4.58 (m, 2H), 4.57-4.47 (m, 2H), 3.91- 3.11 (m, 5H), 2.44-1.87 |
| | | | | | (m, 3H), 1.76-1.61 (m, |
| | | | | | 1H). MS 452.22 (M + H). |
| 230 | 4-([1,1′-biphenyl]- 3-yl)-6-(4-(oxetan- 3-yl)piperazine-1- carbonyl)quinoline- 2-carbaldehyde | 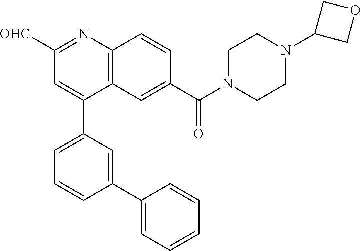 [SMILES: O=Cc1cc(-c2cccc(-c3ccccc3)c2)c2cc(C(=O)N3CCN(C4COC4)CC3)ccc2n1] | Production Ex. 224 | Production Ex. 275 | 1H NMR (400 MHz) δ 10.29 (s, 1H), 8.38 (d, J = 8.7 Hz, 1H), 8.10-8.07 (m, 2H), 7.85 (dd, J = 8.7, 1.8 Hz, 1H), 7.80-7.75 (m, 1H), 7.70 (t, J = 1.8 Hz, 1H), 7.67-7.61 (m, 3H), 7.51-7.44 (m, 3H), 7.43-7.37 (m, 1H), 4.63 (t, J = 6.6 Hz, 2H), 4.55 (t, J = 6.2 Hz, 2H), 3.91-3.33 (m, 5H), 2.47-2.04 (m, 4H). MS 478.22 (M + H). |
| 231 | 4-([1,1′-biphenyl]- 4-yl)-6-(4-(oxetan- 3-yl)piperazine-1- carbonyl)quinoline- 2-carbaldehyde | 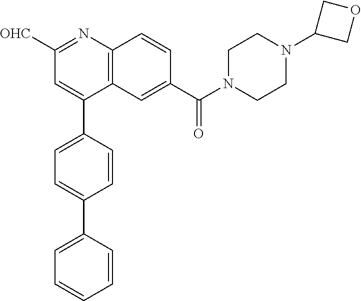 [SMILES: O=Cc1cc(-c2ccc(-c3ccccc3)cc2)c2cc(C(=O)N3CCN(C4COC4)CC3)ccc2n1] | Production Ex. 224 | Production Ex. 276 | 1H NMR (400 MHz) δ 10.29 (s, 1H), 8.36 (s, 1H), 8.14 (d, J = 1.8 Hz, 1H), 8.07 (s, 1H), 7.84 (dd, J = 8.7, 1.7 Hz, 1H), 7.81- 7.77 (m, 2H), 7.71-7.67 (m, 2H), 7.62-7.57 (m, 2H), 7.55-7.49 (m, 2H), 7.45-7.40 (m, 1H), 4.65 (t, J = 6.5 Hz, 2H), 4.59 (t, J = 6.2 Hz, 2H), 3.95-3.35 (m, 5H), 2.42 (s, 4H). MS 478.20 (M + H). |
| 232 | tert-butyl ((4- ([1,1′-biphenyl]-4- yl)-2- formylquinolin-6- yl)methyl)(tetrahydro- 2H-pyran-4- yl)carbamate | 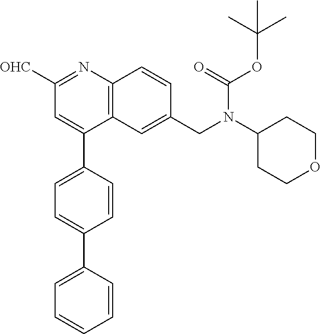 [SMILES: CC(C)(C)OC(=O)N(Cc1ccc2nc(C=O)cc(-c3ccc(-c4ccccc4)cc3)c2c1)C1CCOCC1] | Production Ex. 224 | Production Ex. 416 | 1H NMR (400 MHz) δ 10.27 (s, 1H), 8.28 (d, J = 8.7 Hz, 1H), 8.02 (s, 1H), 7.91-7.87 (m, 1H), 7.81- 7.66 (m, 5H), 7.58 (d, J = 8.0 Hz, 2H), 7.54-7.47 (m, 2H), 7.45-7.39 (m, 1H), 4.72-4.19 (m, 3H), 3.93 (dd, J = 11.7, 4.5 Hz, 2H), 3.50-3.25 (m, 2H), 1.76-1.17 (m, 13H). MS 523.24 (M + H). |
| 233 | tert-butyl ((4- ([1,1′-biphenyl]-3- yl)-2- formylquinolin-6- yl)methyl)(tetrahydro- 2H-pyran-4- yl)carbamate | 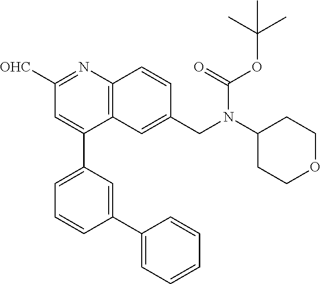 [SMILES: CC(C)(C)OC(=O)N(Cc1ccc2nc(C=O)cc(-c3cccc(-c4ccccc4)c3)c2c1)C1CCOCC1] | Production Ex. 224 | Production Ex. 417 | 1H NMR (400 MHz) δ 10.27 (s, 1H), 8.27 (s, 1H), 8.03 (s, 1H), 7.85-7.81 (m, 1H), 7.77-7.67 (m, 3H), 7.66-7.59 (m, 3H), 7.51-7.44 (m, 3H), 7.42- 7.36 (m, 1H), 4.70-4.16 (m, 3H), 3.83 (dd, J = 11.4, 4.6 Hz, 2H), 3.40-3.16 (m, 2H), 1.76-0.96 (m, 13H). MS 523.24 (M + H). |
| 234 | tert-butyl ((2- formyl-4- (naphthalen-2- yl)quinolin-6- yl)methyl(tetrahydro- 2H-pyran-4- yl)carbamate | 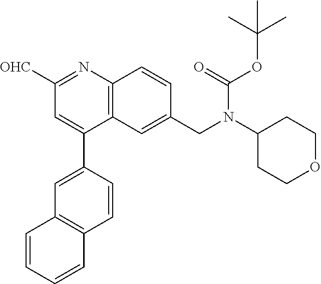 [SMILES: CC(C)(C)OC(=O)N(Cc1ccc2nc(C=O)cc(-c3ccc4ccccc4c3)c2c1)C1CCOCC1] | Production Ex. 224 | Production Ex. 418 | 1H NMR (400 MHz) δ 10.29 (s, 1H), 8.30 (d, J = 8.7 Hz, 1H), 8.07 (s, 1H), 8.01 (d, J = 8.5 Hz, 1H), 7.99-7.94 (m, 2H), 7.93- 7.89 (m, 1H), 7.86-7.82 (m, 1H), 7.74 (dd, J = 8.8, 2.0 Hz, 1H), 7.63-7.56 (m, 3H), 4.71-4.16 (m, 3H), 3.90 (dd, J = 11.5, 4.5 Hz, 2H), 3.44-3.24 (m, 2H), 1.75-1.07 (m, 13H). MS 497.23 (M + H). |
| 235 | tert-butyl ((2- formyl-[4,8′- biquinolin]-6- yl)methyl)(tetrahydro- 2H-pyran-4- yl)carbamate | 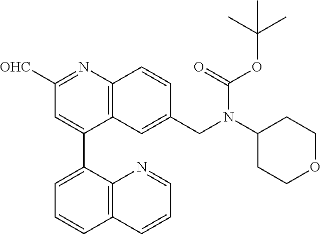 [SMILES: CC(C)(C)OC(=O)N(Cc1ccc2nc(C=O)cc(-c3cccc4cccnc34)c2c1)C1CCOCC1] | Production Ex. 224 | Production Ex. 419 | 1H NMR (399 MHz) δ 10.30 (s, 1H), 8.75 (dd, J = 4.1, 1.8 Hz, 1H), 8.31- 8.26 (m, 2H), 8.07 (s, 1H), 8.04-7.99 (m, 1H), 7.73- 7.63 (m, 3H), 7.45 (dd, J = 8.3, 4.2 Hz, 1H), 7.23 (d, J = 1.8 Hz, 1H), 4.55-3.97 (m, 3H), 3.91-3.77 (m, 2H), 3.41-3.11 (m, 2H), 1.67-0.96 (m, 13H). MS 498.20 (M + H). |
| 236 | 4-(naphthalen-2- yl)-6-(4-(oxetan-3- yl)piperazine-1- carbonyl)quinoline- 2-carbaldehyde | 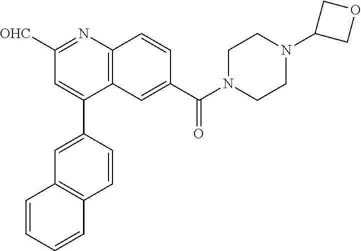 [SMILES: O=Cc1cc(-c2ccc3ccccc3c2)c2cc(C(=O)N3CCN(C4COC4)CC3)ccc2n1] | Production Ex. 224 | Production Ex. 281 | 1H NMR (400 MHz) δ 10.29 (s, 1H), 8.42-8.36 (m, 1H), 8.15-7.82 (m, 7H), 7.67-7.57 (m, 3H), 4.63 (t, J = 6.5 Hz, 2H), 4.57 (t, J = 6.2 Hz, 2H), 3.91-3.71 (m, 2H), 3.53- 3.36 (m, 3H), 2.46-2.13 (m, 4H). MS 452.22 (M + H). |
| 237 | 4-(3-(4,4,5,5- tetramethyl-1,3,2- dioxaborolan-2- yl)phenyl)-1-trityl- 1H-pyrazole | 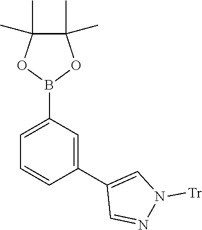 | | Production Ex. 221 | 1H NMR (500 MHz) δ 7.95 (d, J = 0.8 Hz, 1H), 7.83-7.81 (m, 1H), 7.66- 7.62 (m, 2H), 7.53-7.49 (m, 1H), 7.34-7.29 (m, 10H), 7.21-7.15 (m, 6H), 1.34 (s, 12H). MS 513.48 (M + H). |
| 238 | (2-methyl-4-(1H- pyrazol-4- yl)quinolin-6- yl)(morpholino) methanone | 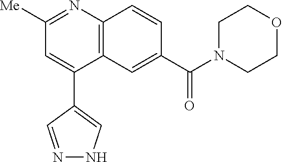 [SMILES: Cc1cc(-c2cn[nH]c2)c2cc(C(=O)N3CCOCC3)ccc2n1] | | Production Ex. 34 CAS No. 1188405- 87- 9 (Reagent Supplier 13) | 1H NMR (500 MHz) δ 8.20 (d, J = 1.9 Hz, 1H), 8.10 (d, J = 8.6 Hz, 1H), 7.93 (s, 2H), 7.69 (dd, J = 8.6, 1.9 Hz, 1H), 7.33 (s, 1H), 3.96-3.36 (m, 8H), 2.78 (s, 3H). MS 323.20 (M + H). |
| 239 | ethyl 4-bromo-2- methylquinoline-6- carboxylate | 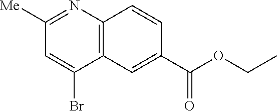 [SMILES: CCOC(=O)c1ccc2nc(C)cc(Br)c2c1] | | Production Ex. 66 | 1H NMR (500 MHz) δ 8.86 (d, J = 1.9 Hz, 1H), 8.31 (dd, J = 8.8, 1.9 Hz, 1H), 8.03 (d, J = 8.7 Hz, 1H), 7.66 (s, 1H), 4.46 (q, J = 7.2 Hz, 2H), 2.73 (s, 3H), 1.47-1.43 (m, 3H). MS 293.95 (M + H). |
| 240 | ethyl 2-methyl-4- (pyrimidin-5- yl)quinoline-6- carboxylate | 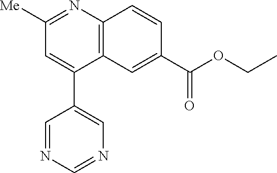 [SMILES: CCOC(=O)c1ccc2nc(C)cc(-c3cncnc3)c2c1] | | Production Ex. 239 CAS NO. 73183- 34- 3 (Reagent Supplier 4) | 1H NMR (500 MHz) δ 9.41 (s, 1H), 8.94 (s, 2H), 8.44 (dd, J = 1.9, 0.6 Hz, 1H), 8.36 (dd, J = 8.8, 1.8 Hz, 1H), 8.17 (dd, J = 8.9, 0.6 Hz, 1H), 7.31 (s, 1H), 4.41 (q, J = 7.1 Hz, 2H), 2.85 (s, 3H), 1.39 (t, J = 7.1 Hz, 3H). MS 294.22 (M + H). |
| 241 | ethyl 2-methyl-4- (1-methyl-1H- 1,2,4-triazol-3- yl)quinoline-6- carboxylate | 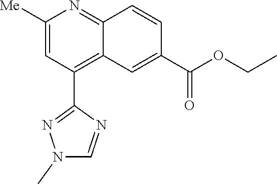 [SMILES: CCOC(=O)c1ccc2nc(C)cc(-c3ncn(C)n3)c2c1] | Production Ex. 240 | Production Ex. 239 CAS NO. 73183- 34- 3 (Reagent Supplier 4) | 1H NMR (500 MHz) δ 9.87 (d, J = 1.8 Hz, 1H), 8.31 (dd, J = 8.8, 1.9 Hz, 1H), 8.25 (s, 1H), 8.11 (d, J = 8.8 Hz, 1H), 8.04 (s, 1H), 4.45 (q, J = 7.1 Hz, 2H), 4.11 (s, 3H), 2.83 (s, 3H), 1.45 (t, J = 7.1 Hz, 3H). MS 297.23 (M + H). |
| 242 | ethyl 2-methyl-4- (1-methyl-1H- imidazol-2- yl)quinoline-6- carboxylate | 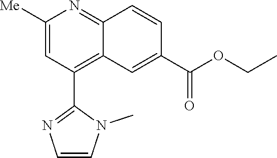 [SMILES: CCOC(=O)c1ccc2nc(C)cc(-c3nccn3C)c2c1] | Production Ex. 240 | Production Ex. 239 CAS NO. 73183- 34- 3 (Reagent Supplier 4) | MS 296.23 (M + H). |
| 243 | ethyl 2-methyl-4- (pyrimidin-2- yl)quinoline-6- carboxylate | 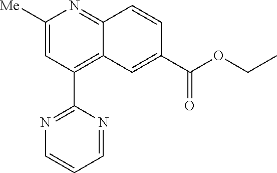 [SMILES: CCOC(=O)c1ccc2nc(C)cc(-c3ncccn3)c2c1] | Production Ex. 240 | Production Ex. 239 CAS NO. 73183- 34- 3 (Reagent Supplier 4) | 1H NMR (500 MHz) δ 9.41 (dd, J = 2.0, 0.6 Hz, 1H), 9.02 (d, J = 4.9 Hz, 2H), 8.31 (dd, J = 8.8, 1.9 Hz, 1H), 8.14 (dd, J = 8.8, 0.6 Hz, 1H), 7.93 (s, 1H), 7.44 (t, J = 4.9 Hz, 1H), 4.43 (q, J = 7.1 Hz, 2H), 2.85 (s, 3H), 1.42 (t, J = 7.1 Hz, 3H). MS 294.18 (M + H). |
| 244 | ethyl 2-methyl-4- (N-((2- (trimethylsilyl)etho xy)methyl)-1,2,4- triazol-3- yl)quinoline-6- carboxylate | 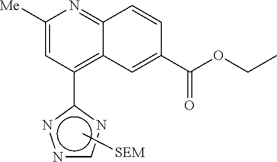 | Production Ex. 240 | Production Ex. 239 Produ ction Ex. 255 CAS NO. 73183- 34- 3 (Reagent Supplier 4) | MS 413.37 (M + H). |
| 245 | ethyl 2-methyl-4- (pyridazin-3- yl)quinoline-6- carboxylate | 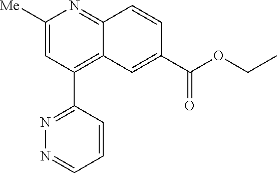 [SMILES: CCOC(=O)c1ccc2nc(C)cc(-c3cccnn3)c2c1] | Production Ex. 240 | Production Ex. 239 CAS NO. 73183- 34- 3 (Reagent Supplier 4) | 1H NMR (500 MHz) δ 9.39 (dd, J = 5.0, 1.7 Hz, 1H), 8.64 (d, J = 1.8 Hz, 1H), 8.34 (dd, J = 8.8, 1.8 Hz, 1H), 8.17 (d, J = 8.8 Hz, 1H), 7.82 (dd, J = 8.5, 1.6 Hz, 1H), 7.72 (dd, J = 8.4, 5.0 Hz, 1H), 7.58 (s, 1H), 4.40 (q, J = 7.1 Hz, 2H), 2.86 (s, 3H), 1.39 (t, J = 7.1 Hz, 3H). MS 294.17 |
| | | | | | (M + H). |
| 246 | ethyl 2-methyl-4- (1-methyl-1H- imidazol-4- yl)quinoline-6- carboxylate | 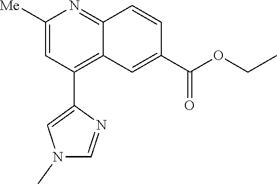 [SMILES: CCOC(=O)c1ccc2nc(C)cc(-c3cn(C)cn3)c2c1] | Production Ex. 240 | Production Ex. 239 CAS NO. 73183- 34- 3 (Reagent Supplier 4) | 1H NMR (500 MHz) δ 9.26 (d, J = 1.9 Hz, 1H), 8.23 (dd, J = 8.8, 1.9 Hz, 1H), 8.04 (d, J = 8.8 Hz, 1H), 7.66 (s, 1H), 7.63 (d, J = 1.3 Hz, 1H), 7.41 (d, J = 1.3 Hz, 1H), 4.41 (q, J = 7.1 Hz, 2H), 3.81 (s, 3H), 2.75 (s, 3H), 1.41 (t, J = 7.1 Hz, 3H). MS 296.16 (M + H). |
| 247 | ethyl 2-methyl-4- (pyrazin-2- yl)quinoline-6- carboxylate | 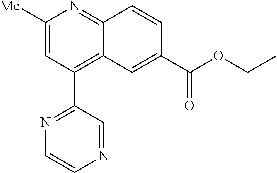 [SMILES: CCOC(=O)c1ccc2nc(C)cc(-c3cnccn3)c2c1] | Production Ex. 240 | Production Ex. 239 CAS NO. 73183- 34- 3 (Reagent Supplier 4) | 1H NMR (500 MHz) δ 8.96 (d, J = 1.6 Hz, 1H), 8.84 (dd, J = 2.5, 1.5 Hz, 1H), 8.81 (d, J = 1.8 Hz, 1H), 8.76 (d, J = 2.5 Hz, 1H), 8.34 (dd, J = 8.8, 1.9 Hz, 1H), 8.16 (d, J = 8.8 Hz, 1H), 7.51 (s, 1H), 4.41 (q, J = 7.1 Hz, 2H), 2.86 (s, 3H), 1.40 (t, J = 7.1 Hz, 3H). MS 294.13 (M + H). |
| 248 | N-((4-([1,1′- biphenyl]-4-yl)-2- methylquinolin-6- yl)methyl)tetrahydro- 2H-pyran-4- amine | 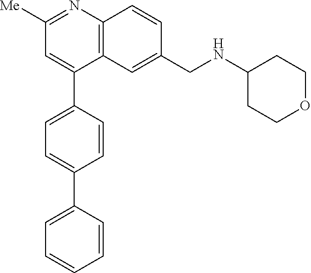 [SMILES: Cc1cc(-c2ccc(-c3ccccc3)cc2)c2cc(CNC3CCOCC3)ccc2n1] | | Production Ex. 277 | 1H NMR (399 MHz) δ 8.07 (d, J = 8.6 Hz, 1H), 7.81 (d, J = 1.9 Hz, 1H), 7.79-7.66 (m, 5H), 7.61- 7.55 (m, 2H), 7.54-7.47 (m, 2H), 7.44-7.37 (m, 1H), 7.27 (s, 1H), 4.00- 3.92 (m, 4H), 3.41-3.31 (m, 2H), 2.82-2.67 (m, 4H), 1.88-1.78 (m, 2H), 1.50-1.37 (m, 2H). MS 409.26 (M + H). |
| 249 | N-((4-([1,1′- biphenyl]-3-yl)-2- methylquinolin-6- yl)methyl)tetrahydro- 2H-pyran-4- amine | 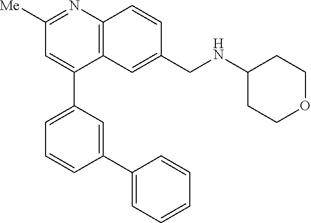 [SMILES: Cc1cc(-c2cccc(-c3ccccc3)c2)c2cc(CNC3CCOCC3)ccc2n1] | Production Ex. 248 | Production Ex. 278 | 1H NMR (400 MHz) δ 8.07 (d, J = 8.7 Hz, 1H), 7.81 (d, J = 1.8 Hz, 1H), 7.75-7.58 (m, 6H), 7.50- 7.44 (m, 3H), 7.41-7.35 (m, 1H), 7.29 (s, 1H), 3.95- 3.87 (m, 4H), 3.35-3.25 (m, 2H), 2.78 (s, 3H), 2.75- 2.66 (m, 1H), 1.84-1.74 (m, 2H), 1.47-1.34 (m, 2H). MS 409.26 (M + H). |
| 250 | N-((2-methyl-4- (naphthalen-2- yl)quinolin-6- yl)methyl)tetrahydro- 2H-pyran-4- amine | 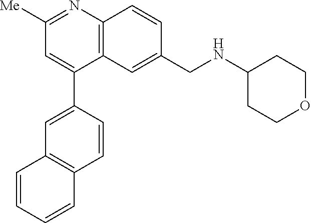 [SMILES: Cc1cc(-c2ccc3ccccc3c2)c2cc(CNC3CCOCC3)ccc2n1] | Production Ex. 248 | Production Ex. 279 | 1H NMR (399 MHz) δ 8.09 (d, J = 8.6 Hz, 1H), 8.01-7.89 (m, 4H), 7.76 (d, J = 1.9 Hz, 1H), 7.71 (dd, J = 8.6, 1.9 Hz, 1H), 7.63-7.55 (m, 3H), 7.32 (s, 1H), 3.97-3.88 (m, 4H), 3.38-3.29 (m, 2H), 2.80 (s, 3H), 2.74-2.65 (m, 1H), 1.83-1.75 (m, 2H), 1.46-1.34 (m, 2H). MS 383.22 (M + H). |
| 251 | N-((2-methyl-[4,8′- biquinolin]-6- yl)methyl)tetrahydro- 2H-pyran-4- amine | 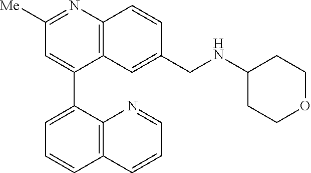 [SMILES: Cc1cc(-c2cccc3cccnc23)c2cc(CNC3CCOCC3)ccc2n1] | Production Ex. 248 | Production Ex. 280 | 1H NMR (399 MHz) δ 8.81 (dd, J = 4.1, 1.8 Hz, 1H), 8.29 (dd, J = 8.2, 1.8 Hz, 1H), 8.07 (d, J = 8.7 Hz, 1H), 7.99 (dd, J = 6.8, 3.1 Hz, 1H), 7.72-7.66 (m, 2H), 7.64 (dd, J = 8.7, 1.9 Hz, 1H), 7.45 (dd, J = 8.3, 4.1 Hz, 1H), 7.34 (s, 1H), 7.19 (d, J = 1.9 Hz, 1H), 3.92-3.80 (m, 2H), |
| | | | | | 3.77 (s, 2H), 3.32-3.17 |
| | | | | | (m, 2H), 2.80 (s, 3H), 2.63- |
| | | | | | 2.52 (m, 1H), 1.74-1.53 |
| | | | | | (m, 2H), 1.38-1.17 (m, |
| | | | | | 2H). MS 384.21 (M + H). |
| 252 | tert-butyl 3-(2- methyl-6- (morpholine-4- carbonyl)quinolin- 4-yl)piperidine-1- carboxylate | 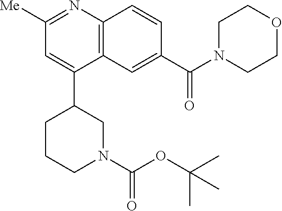 [SMILES: Cc1cc(C2CCCN(C(=O)OC(C)(C)C)C2)c2cc(C(=O)N3CCOCC3)ccc2n1] | | Production Ex. 358 | 1H NMR (500 MHz) δ 8.23 (s, 1H), 8.12 (d, J = 8.5 Hz, 1H), 7.69 (d, J = 8.5 Hz, 1H), 7.23 (s, 1H), 4.45-4.09 (m, 2H), 3.96- 3.41 (m, 9H), 3.04-2.74 (m, 5H), 2.17-2.11 (m, 1H), 1.89-1.67 (m, 3H), 1.50 (s, 9H). MS 440.51 (M + H). |
| 253 | 1-(3-(2-methyl-6- (morpholine-4- carbonyl)quinolin- 4-yl)piperidin-1- yl)ethan-1-one | 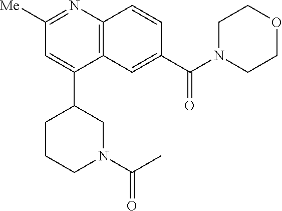 [SMILES: CC(=O)N1CCCC(c2cc(C)nc3ccc(C(=O)N4CCOCC4)cc23)C1] | | Production Ex. 252 | 1H NMR (500 MHz) δ 8.19, 8.16 (each d, each J = 1.8 Hz, combined 1H), 8.10-8.05 (m, 1H), 7.71- 7.66 (m, 1H), 7.23, 7.20 (each s, combined 1H), 4.90-4.75 (m, 1H), 4.06- 3.36 (m, 10H), 3.29-3.13 (m, 1H), 2.78-2.59 (m, 4H), 2.23-2.12 (m, 4H), 1.99-1.67 (m, 3H). MS 382.33 (M + H). |
| 254 | 1-(6-(2- methylquinolin-4- yl)-3,4- dihydroisoquinolin- 2(1H)-yl)ethan-1- one | 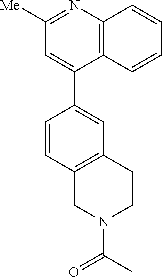 [SMILES: CC(=O)N1CCc2cc(-c3cc(C)nc4ccccc34)ccc2C1] | Production Ex. 253 | Production Ex. 359 | 1H NMR (500 MHz) δ 8.07 (d, J = 8.5 Hz, 1H), 7.82 (d, J = 8.5 Hz, 1H), 7.69-7.65 (m, 1H), 7.44- 7.40 (m, 1H), 7.35-7.23 (m, 3H), 7.19, 7.19 (each s, combined 1H), 4.83, 4.71 (each s, combined 2H), 3.88, 3.74 (each t, each J = 6.0 Hz, combined 2H), 2.98, 2.92 (each t, each J = 6.0 Hz, combined 2H), 2.76 (s, 3H), 2.22, 2.20 (each s, combined 3H). MS 317.25 (M + H). |
| 255 | 3-bromo-N-((2- (trimethylsilyl)etho xy)methyl)-1,2,4- triazole | 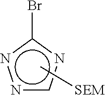 | | | 1H NMR (500 MHz) δ 8.14, 7.93 (each s, combined 1H), 5.51, 5.46 (each s, combined 2H), 3.69-3.63 (m, 2H), 0.97- 0.91 (m, 2H), 0.02-0.00 |
| | | | | | (m, 9H). MS 278.05 |
| | | | | | (M + H). |
| 256 | 2-methyl-4-(1-((2- (trimethylsilyl) ethoxy)methyl)-1H- pyrazol-4- yl)quinoline | 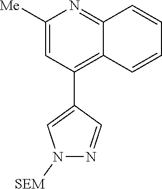 [SMILES: Cc1cc(-c2cnn(COCC[Si](C)(C)C)c2)c2ccccc2n1] | | Production Ex. 360 | 1H NMR (500 MHz) δ 8.13-8.04 (m, 2H), 7.92 (s, 1H), 7.87 (s, 1H), 7.73- 7.68 (m, 1H), 7.53-7.47 (m, 1H), 7.28 (s, 1H), 5.55 (s, 2H), 3.72-3.65 (m, 2H), 2.76 (s, 3H), 1.01- 0.94 (m, 2H), 0.01 (s, 9H). MS 340.26 (M + H). |
| 257 | 2-methyl-6- (morpholine-4- carbonyl)quinolin- 5-yl trifluoromethane- sulfonate | 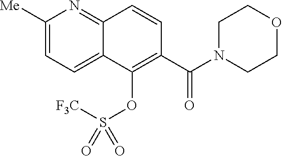 [SMILES: Cc1ccc2c(OS(=O)(=O)C(F)(F)F)c(C(=O)N3CCOCC3)ccc2n1] | | Production Ex. 282 | 1H NMR (500 MHz) δ 8.35 (d, J = 8.7 Hz, 1H), 8.11 (d, J = 8.7 Hz, 1H), 7.65 (d, J = 8.6 Hz, 1H), 7.50 (d, J = 8.8 Hz, 1H), 3.90-3.57 (m, 6H), 3.42 (t, J = 4.9 Hz, 2H), 2.80 (s, 3H). MS 405.0 (M + H). |
| 258 | 2-methyl-6- (morpholine-4- carbonyl)quinolin- 8-yl trifluoromethane- sulfonate | 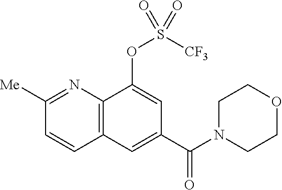 [SMILES: Cc1ccc2cc(C(=O)N3CCOCC3)cc(OS(=O)(=O)C(F)(F)F)c2n1] | Production Ex. 257 | Production Ex. 283 | 1H NMR (500 MHz) δ 8.13 (d, J = 8.5 Hz, 1H), 7.92-7.90 (m, 1H), 7.63- 7.60 (m, 1H), 7.46 (d, J = 8.4 Hz, 1H), 4.02-3.36 (m, 8H), 2.82 (s, 3H). MS 405.2 (M + H). |
| 259 | methyl 2-methyl-3- (((trifluoromethyl)s ulfonyl)oxy)quinoline- 6-carboxylate | 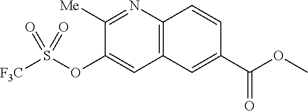 [SMILES: COC(=O)c1ccc2nc(C)c(OS(=O)(=O)C(F)(F)F)cc2c1] | Production Ex. 257 | CAS No. 2440087- 41-0 (Synthetic Literature 13) | 1H NMR (500 MHz) δ 8.60 (d, J = 1.9 Hz, 1H), 8.35 (dd, J = 8.8, 1.9 Hz, 1H), 8.14 (s, 1H), 8.11 (d, J = 8.8 Hz, 1H), 4.01 (s, 3H), 2.83 (s, 3H). MS 350.1 (M + H). |
| 260 | (2-methyl-5- (naphthalen-1- yl)quinolin-6- yl)(morpholino) methanone | 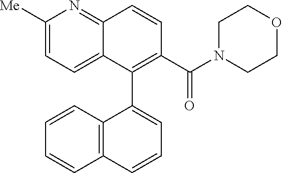 [SMILES: Cc1ccc2c(-c3cccc4ccccc34)c(C(=O)N3CCOCC3)ccc2n1] | | Production Ex. 257 | 1H NMR (500 MHz) δ 8.17 (d, J = 8.7 Hz, 1H), 8.01-7.95 (m, 2H), 7.74- 7.66 (m, 2H), 7.62 (t, J = 7.6 Hz, 1H), 7.53-7.45 (m, 2H), 7.31-7.22 (m, 1H), 7.12 (d, J = 8.6 Hz, 2H), 3.62-3.46 (m, 2H), 3.31-3.16 (m, 2H), 3.03- 2.90 (m, 2H), 2.83-2.70 (m, 5H). MS 383.1 |
| | | | | | (M + H). |
| 261 | (2-methyl-8- (naphthalen-1- yl)quinolin-6- yl)(morpholino) methanone | 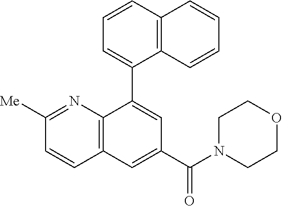 [SMILES: Cc1ccc2cc(C(=O)N3CCOCC3)cc(-c3cccc4ccccc34)c2n1] | Production Ex. 260 | Production Ex. 258 | MS 383.3 (M + H). |
| 262 | (2-methyl-4-(2-(1- methyl-1H- pyrazol-4- yl)phenyl)quinolin-6- yl)(morpholino) methanone | 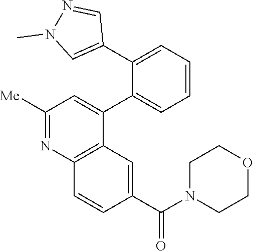 [SMILES: Cc1cc(-c2ccccc2-c2cnn(C)c2)c2cc(C(=O)N3CCOCC3)ccc2n1] | | Production Ex. 34 CAS No. 1638289- 93-6 (Synthetic Literature 11) | 1H NMR (500 MHz) δ 8.08 (d, J = 8.6 Hz, 1H), 7.66 (dd, J = 8.6, 1.9 Hz, 1H), 7.60 (dd, J = 7.9, 1.3 Hz, 1H), 7.54-7.46 (m, 2H), 7.41-7.34 (m, 1H), 7.30-7.23 (m, 2H), 7.12 (s, 1H), 6.73 (s, 1H), 3.86- 3.11 (m, 11H), 2.77 (s, 3H). MS 413.4 (M + H). |
| 263 | 2-methyl-3- (naphthalen-1- yl)quinoline-6- carbonitrile | 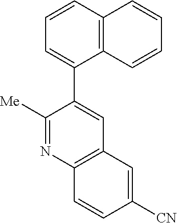 [SMILES: Cc1nc2ccc(C#N)cc2cc1-c1cccc2ccccc12] | | Production Ex. 210 | 1H NMR (500 MHz) δ 8.21 (d, J = 4.9 Hz, 1H), 8.20 (d, J = 5.7 Hz, 1H), 8.07 (s, 1H), 7.98 (d, J = 4.9 Hz, 1H), 7.96 (d, J = 4.9 Hz, 1H), 7.88 (dd, J = 8.8, 1.8 Hz, 1H), 7.60 (t, J = 7.5 Hz, 1H), 7.54 (t, J = 7.5 Hz, 1H), 7.45-7.39 (m, 2H), 7.34 (d, J = 8.5 Hz, 1H), 2.47 (s, 3H). MS 295.2 (M + H). |
| 264 | 2-methyl-3- (naphthalen-1-yl)- 6-(1H-tetrazo1-5- yl)quinoline | 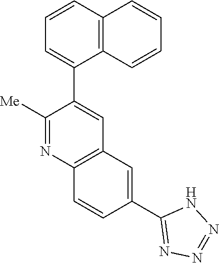 [SMILES: Cc1nc2ccc(-c3nnn[nH]3)cc2cc1-c1cccc2ccccc12] | | Production Ex. 263 | 1H NMR (500 MHz, dimethylsulfoxide-d6) δ 8.97 (s, 1H), 8.91 (br s, 1H), 8.66 (d, J = 8.8 Hz, 1H), 8.56 (d, J = 8.8 Hz, 1H), 8.14 (d, J = 8.2 Hz, 1H), 8.11 (d, J = 8.2 Hz, 1H), 7.72 (t, J = 7.2 Hz, 1H), 7.65-7.58 (m, 2H), 7.55-7.48 (m, 2H), 2.55 (s, 3H). MS 338.18 (M + H). |
| 265 | 2-methyl-3- (naphthalen-1-yl)- 6-(N-((2- (trimethylsilyl)etho xy)methyl)tetrazol- 5-yl)quinoline | 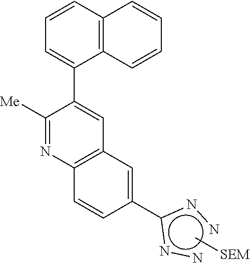 | | Production Ex. 264 | MS 468.3 (M + H). |
| 266 | 4-((2- methylquinolin-4- yl)methyl)morpholine | 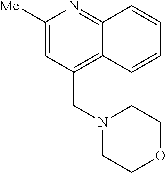 [SMILES: Cc1cc(CN2CCOCC2)c2ccccc2n1] | | CAS NO. 6760-22- 1 (Reagent Supplier 3) | 1H NMR (500 MHz) δ 8.19 (d, J = 8.3 Hz, 1H), 8.03 (d, J = 8.3 Hz, 1H), 7.67 (t, J = 8.0 Hz, 1H), 7.49 (t, J = 8.0 Hz, 1H), 7.29 (s, 1H), 3.86 (s, 2H), 3.75-3.70 (m, 4H), 2.73 (s, 3H), 2.55-2.50 (m, 4H). MS 243.2 (M + H). |
| 267 | 4-((2- methylquinolin-4- yl)methyl)morpholin- 3-one | 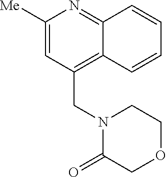 [SMILES: Cc1cc(CN2CCOCC2=O)c2ccccc2n1] | | CAS NO. 6760-22-1 (Reagent Supplier 3) CAS NO. 1803611- 80-4 (Reagent Supplier 5) | 1H NMR (500 MHz) δ 8.06 (d, J = 8.4 Hz, 1H), 8.02 (d, J = 8.4 Hz, 1H), 7.72 (t, J = 8.4 Hz, 1H), 7.54 (t, J = 8.4 Hz, 1H), 7.14 (s, 1H), 5.09 (s, 2H), 4.32 (s, 2H), 3.86 (t, J = 5.3 Hz, 2H), 3.27 (t, J = 5.3 Hz, 2H), 2.75 (s, 3H). MS 257.2 (M + H). |
| 268 | N-((2- methylquinolin-4- yl)methyl)tetrahydro- 2H-pyran-4- amine | 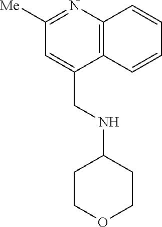 [SMILES: Cc1cc(CNC2CCOCC2)c2ccccc2n1] | Production Ex. 72 | CAS NO. 6760-22-1 (Reagent Supplier 3) CAS NO. 38041-19- 9 (Reagent Supplier 13) | 1H NMR (500 MHz) δ 8.03 (d, J = 8.5 Hz, 1H), 7.96 (d, J = 8.5 Hz, 1H), 7.64 (t, J = 8.5 Hz, 1H), 7.47 (t, J = 8.5 Hz, 1H), 7.31 (s, 1H), 4.18 (s, 2H), 4.01-3.96 (m, 2H), 3.45- 3.36 (m, 2H), 2.84-2.76 (m, 1H), 2.71 (s, 3H), 1.93- 1.88 (m, 2H), 1.54-1.44 (m, 2H). MS 257.2 (M + H). |
| 269 | tert-butyl ((4-(2- acetyl-1,2,3,4- tetrahydroisoquinolin- 6-yl)-2- methylquinolin-6- yl)methyl)(tetrahydro- 2H-pyran-4- yl)carbamate | 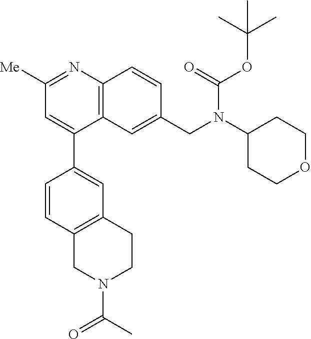 [SMILES: CC(=O)N1CCc2cc(-c3cc(C)nc4ccc(CN(C(=O)OC(C)(C)C)C5CCOCC5)cc34)ccc2C1] | | Production Ex. 420 CAS NO. 1181691- 47- 3 (Reagent Supplier 9) | 1H NMR (500 MHz) δ 8.05-8.00 (m, 1H), 7.66- 7.61 (m, 1H), 7.58 (d, J = 8.8 Hz, 1H), 7.33-7.17 (m, 4H), 4.84, 4.73 (each s, combined 2H), 4.59-4.18 (m, 3H), 3.94-3.84 (m, 2H), 3.80-3.67 (m, 2H), 3.46-3.25 (m, 2H), 3.03- 2.90 (m, 2H), 2.76 (s, 3H), 2.25, 2.23 (each s, combined 3H), 1.74-1.17 (m, 13H). MS 530.4 (M + H). |
| 270 | tert-butyl ((2- methyl-4-(3-(1- methyl-1H- pyrazol-4- yl)phenyl)quinolin- 6- yl)methyl)(tetrahydro- 2H-pyran-4- yl)carbamate | 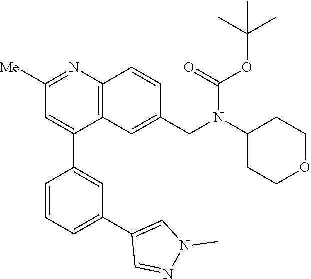 [SMILES: Cc1cc(-c2cccc(-c3cnn(C)c3)c2)c2cc(CN(C(=O)OC(C)(C)C)C3CCOCC3)ccc2n1] | Production Ex. 269 | Production Ex. 420 CAS NO. 1534350- 44- 1 (Reagent Supplier 16) | 1H NMR (500 MHz) δ 8.05 (d, J = 8.7 Hz, 1H), 7.79 (s, 1H), 7.72-7.66 (m, 2H), 7.61-7.56 (m, 2H), 7.55-7.47 (m, 2H), 7.32-7.29 (m, 1H), 7.25 (s, 1H), 4.63-4.15 (m, 3H), 3.96 (s, 3H), 3.87 (dd, J = 11.4, 4.5 Hz, 2H), 3.41- 3.23 (m, 2H), 2.78 (s, 3H), 1.79-1.02 (m, 13H). MS 513.4 (M + H). |
| 271 | tert-butyl ((4-(2- acetyl-1,2,3,4- tetrahydroisoquinolin- 7-yl)-2- methylquinolin-6- yl)methyl)(tetrahydro- 2H-pyran-4- yl)carbamate | 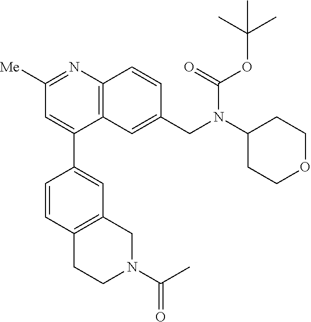 [SMILES: CC(=O)N1CCc2ccc(-c3cc(C)nc4ccc(CN(C(=O)OC(C)(C)C)C5CCOCC5)cc34)cc2C1] | Production Ex. 269 | Production Ex. 420 CAS NO 937591-29- 2 (Reagent Supplier 9) | 1H NMR (500 MHz) δ 8.03 (dd, J = 8.6, 3.0 Hz, 1H), 7.68-7.54 (m, 2H), 7.36-7.17 (m, 4H), 4.82, 4.72 (each s, combined 2H), 4.60-4.20 (m, 3H), 3.96-3.75 (m, 4H), 3.45- 3.26 (m, 2H), 3.02, 2.96 (each t, each J = 5.9 Hz, combined 2H), 2.76 (s, 3H), 2.23, 2.23 (each s, combined 3H), 1.68 (s, 13H). MS 530.5 (M + H). |
| 272 | (4-([1,1′-biphenyl]- 2-yl)-2- methylquinolin-6- yl)(4- methylpiperazin-1- yl)methanone | 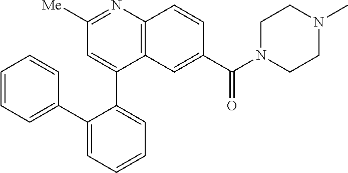 [SMILES: Cc1cc(-c2ccccc2-c2ccccc2)c2cc(C(=O)N3CCN(C)CC3)ccc2n1] | Production Ex. 18 | Production Ex. 94 | 1H NMR (500 MHz) 8 7.98 (dd, J = 8.6, 0.6 Hz, 1H), 7.62 (d, J = 1.8 Hz, 1H), 7.59-7.53 (m, 3H), 7.50-7.45 (m, 1H), 7.38- 7.35 (m, 1H), 7.13 (s, 1H), 7.09-7.01 (m, 5H), 3.95- 3.58 (m, 2H), 3.38-3.14 (m, 2H), 2.67 (s, 3H), 2.57- 2.15 (m, 7H). MS 422.34 (M + H). |
| 273 | tert-butyl 1-(4- ([1,1′-biphenyl]-2- yl)-2- methylquinoline-6- carbonyl)piperidine- 4-carboxylate | 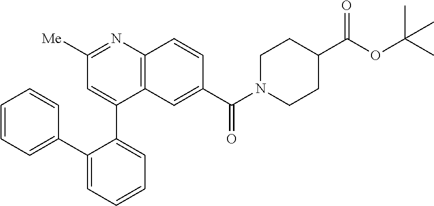 [SMILES: Cc1cc(-c2ccccc2-c2ccccc2)c2cc(C(=O)N3CCC(C(=O)OC(C)(C)C)CC3)ccc2n1] | Production Ex. 18 | Production Ex. 94 CAS No. 138007-24- 6 (Reagent Supplier 6) | MS 507.41 (M + H). |
| 274 | (2-methyl-4- (naphthalen-1- yl)quinolin-6- yl)(4-(oxetan-3- yl)piperazin-1- yl)methanone | 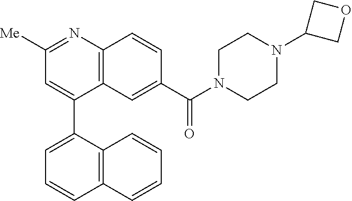 [SMILES: Cc1cc(-c2cccc3ccccc23)c2cc(C(=O)N3CCN(C4COC4)CC3)ccc2n1] | Production Ex. 18 | Production Ex. 216 CAS No. 1254115- 23- 5 (Reagent Supplier 15) | 1H NMR (400 MHz) δ 8.16 (d, J = 8.7 Hz, 1H), 8.01 (dd, J = 8.3, 1.2 Hz, 1H), 7.97 (d, J = 8.2 Hz, 1H), 7.73 (dd, J = 8.6, 2.1 Hz, 1H), 7.62 (dd, J = 8.3, 7.0 Hz, 1H), 7.53-7.43 (m, 2H), 7.39 (s, 1H), 7.33 (d, J = 1.8 Hz, 1H), 7.32- 7.29 (m, 2H), 4.64-4.58 (m, 2H), 4.56-4.48 (m, 2H), 3.90-3.08 (m, 5H), 2.84 (s, 3H), 2.41-1.49 |
| | | | | | (m, 4H). MS 438.27 |
| | | | | | (M + H). |
| 275 | (4-([1,1′-biphenyl]- 3-yl)-2- methylquinolin-6- yl)(4-(oxetan-3- yl)piperazin-1- yl)methanone | 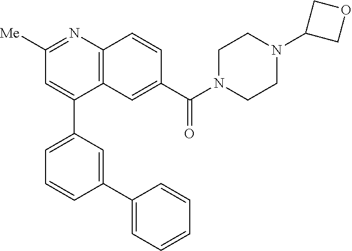 [SMILES: Cc1cc(-c2cccc(-c3ccccc3)c2)c2cc(C(=O)N3CCN(C4COC4)CC3)ccc2n1] | Production Ex. 18 | Production Ex. 215 CAS No. 1254115- 23- 5 (Reagent Supplier 15) | 1H NMR (400 MHz) δ 8.12 (d, J = 8.7 Hz, 1H), 7.96 (d, J = 1.8 Hz, 1H), 7.76-7.67 (m, 3H), 7.66- 7.58 (m, 3H), 7.50-7.44 (m, 3H), 7.42-7.36 (m, 1H), 7.35 (s, 1H), 4.62 (t, J = 6.6 Hz, 2H), 4.55 (t, J = 6.2 Hz, 2H), 3.93-3.32 (m, 5H), 2.81 (s, 3H), 2.48- 2.01 (m, 4H). MS 464.23 (M + H). |
| 276 | (4-([1,1′-biphenyl]- 4-yl)-2- methylquinolin-6- yl)(4-(oxetan-3- yl)piperazin-1- yl)methanone | 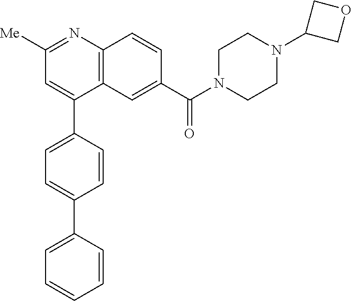 [SMILES: Cc1cc(-c2ccc(-c3ccccc3)cc2)c2cc(C(=O)N3CCN(C4COC4)CC3)ccc2n1] | Production Ex. 18 | Production Ex. 217 CAS No. 1254115- 23- 5 (Reagent Supplier 15) | 1H NMR (400 MHz) δ 8.12 (d, J = 8.7 Hz, 1H), 8.01 (d, J = 1.9 Hz, 1H), 7.78-7.74 (m, 2H), 7.72- 7.66 (m, 3H), 7.59-7.48 (m, 4H), 7.44-7.39 (m, 1H), 7.34 (s, 1H), 4.68- 4.55 (m, 4H), 3.96-3.35 (m, 5H), 2.81 (s, 3H), 2.49- 2.13 (m, 4H). MS 464.26 (M + H). |
| 277 | 4-([1,1′-biphenyl]- 4-yl)-2-methyl-N- (tetrahydro-2H- pyran-4- yl)quinoline-6- carboxamide | 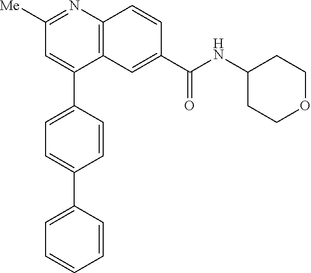 [SMILES: Cc1cc(-c2ccc(-c3ccccc3)cc2)c2cc(C(=O)NC3CCOCC3)ccc2n1] | Production Ex. 18 | Production Ex. 217 CAS No. 38041- 19- 9 (Reagent Supplier 13) | 1H NMR (400 MHz) δ 8.44 (d, J = 1.9 Hz, 1H), 8.14 (d, J = 8.7 Hz, 1H), 7.99 (dd, J = 8.9, 2.0 Hz, 1H), 7.80-7.76 (m, 2H), 7.71-7.66 (m, 2H), 7.62- 7.57 (m, 2H), 7.53-7.48 (m, 2H), 7.44-7.38 (m, 1H), 7.35 (s, 1H), 6.03 (d, J = 7.7 Hz, 1H), 4.27-4.14 (m, 1H), 4.04-3.94 (m, 2H), 3.58-3.47 (m, 2H), 2.82 (s, 3H), 2.06-1.96 (m, 2H), 1.65-1.50 (m, 2H). MS 423.25 (M + H). |
| 278 | 4-([1,1′-biphenyl]- 3-yl)-2-methyl-N- (tetrahydro-2H- pyran-4- yl)quinoline-6- carboxamide | 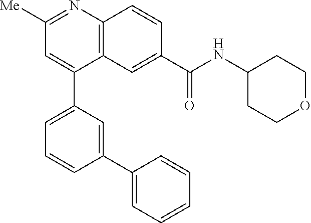 [SMILES: Cc1cc(-c2cccc(-c3ccccc3)c2)c2cc(C(=O)NC3CCOCC3)ccc2n1] | Production Ex. 18 | Production Ex. 215 CAS No. 38041- 19- 9 (Reagent Supplier 13) | 1H NMR (400 MHz) δ 8.41 (d, J = 2.1 Hz, 1H), 8.14 (d, J = 8.8 Hz, 1H), 8.00 (dd, J = 8.9, 2.0 Hz, 1H), 7.76-7.70 (m, 2H), 7.67-7.59 (m, 3H), 7.51- 7.44 (m, 3H), 7.41-7.35 (m, 2H), 6.04 (d, J = 7.8 Hz, 1H), 4.26-4.13 (m, 1H), 4.01-3.93 (m, 2H), 3.56-3.46 (m, 2H), 2.81 (s, 3H), 2.03-1.94 (m, 2H), 1.60-1.47 (m, 2H). MS 423.24 (M + H). |
| 279 | 2-methyl-4- (naphthalen-2-yl)- N-(tetrahydro-2H- pyran-4- yl)quinoline-6- carboxamide | 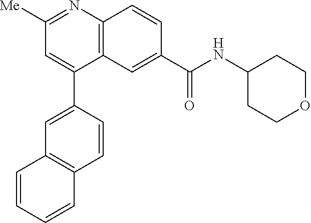 [SMILES: Cc1cc(-c2ccc3ccccc3c2)c2cc(C(=O)NC3CCOCC3)ccc2n1] | Production Ex. 18 | Production Ex. 219 CAS No. 38041- 19- 9 (Reagent Supplier 13) | 1H NMR (400 MHz) δ 8.36 (d, J = 2.0 Hz, 1H), 8.15 (d, J = 8.7 Hz, 1H), 8.04-7.90 (m, 5H), 7.64- 7.55 (m, 3H), 7.40 (s, 1H), 5.99 (d, J = 8.0 Hz, 1H), 4.23-4.11 (m, 1H), 4.00- 3.92 (m, 2H), 3.54-3.45 (m, 2H), 2.83 (s, 3H), 2.01- 1.93 (m, 2H), 1.58-1.43 (m, 2H). MS 397.25 (M + H). |
| 280 | 2-methyl-N- (tetrahydro-2H- pyran-4-yl)-[4,8′- biquinoline]-6- carboxamide | 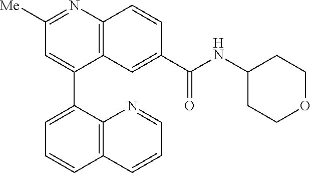 [SMILES: Cc1cc(-c2cccc3cccnc23)c2cc(C(=O)NC3CCOCC3)ccc2n1] | Production Ex. 18 | Production Ex. 218 CAS No. 38041- 19- 9 (Reagent Supplier 13) | 1H NMR (400 MHz) δ 8.81 (dd, J = 4.2, 1.8 Hz, 1H), 8.29 (dd, J = 8.3, 1.8 Hz, 1H), 8.14 (d, J = 8.7 Hz, 1H), 8.01 (dd, J = 7.4, 2.3 Hz, 1H), 7.92 (dd, J = 8.8, 2.1 Hz, 1H), 7.86 (d, J = 2.1 Hz, 1H), 7.74-7.67 (m, 2H), 7.46 (dd, J = 8.3, 4.1 Hz, 1H), 7.42 (s, 1H), 5.87 (d, J = 7.9 Hz, 1H), |
| | | | | | 4.15-4.03 (m, 1H), 3.96- |
| | | | | | 3.88 (m, 2H), 3.51-3.41 |
| | | | | | (m, 2H), 2.83 (s, 3H), 1.96- |
| | | | | | 1.85 (m, 2H), 1.51-1.38 |
| | | | | | (m, 2H). MS 398.23 |
| | | | | | (M + H). |
| 281 | (2-methyl-4- (naphthalen-2- yl)quinolin-6- yl)(4-(oxetan-3- yl)piperazin-1- yl)methanone | 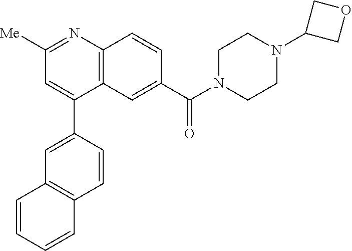 [SMILES: Cc1cc(-c2ccc3ccccc3c2)c2cc(C(=O)N3CCN(C4COC4)CC3)ccc2n1] | Production Ex. 18 | Production Ex. 219 CAS No. 1254115- 23- 5 (Reagent Supplier 15) | 1H NMR (400 MHz) δ 8.14 (d, J = 8.6 Hz, 1H), 8.03-7.86 (m, 5H), 7.72 (dd, J = 8.7, 1.8 Hz, 1H), 7.64-7.55 (m, 3H), 7.39 (s, 1H), 4.62 (t, J = 6.6 Hz, 2H), 4.56 (t, J = 6.2 Hz, 2H), 3.89-3.31 (m, 5H), 2.82 (s, 3H), 2.47-2.06 (m, 4H). MS 438.26 (M + H). |
| 282 | (5-hydroxy-2- methylquinolin-6- yl)(morpholino) methanone | 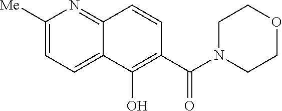 [SMILES: Cc1ccc2c(O)c(C(=O)N3CCOCC3)ccc2n1] | Production Ex. 18 | CAS No. 934611-84- 4 (Synthetic Literature 10) | 1H NMR (500 MHz) δ 11.30 (s, 1H), 8.57 (d, J = 8.5 Hz, 1H), 7.48 (d, J = 8.9 Hz, 1H), 7.45 (d, J = 9.0 Hz, 1H), 7.32 (d, J = 8.5 Hz, 1H), 3.86-3.71 (m, 8H), 2.75 (s, 3H). MS 273.3 (M + H). |
| 283 | (8-hydroxy-2- methylquinolin-6- yl)(morpholino) methanone | 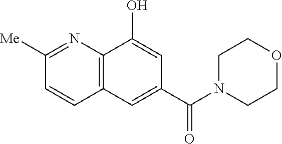 [SMILES: Cc1ccc2cc(C(=O)N3CCOCC3)cc(O)c2n1] | Production Ex. 18 | CAS No. 1668584- 30-2 (Synthetic Literature 12) | 1H NMR (500 MHz) δ 8.06 (d, J = 8.4 Hz, 1H), 7.40-7.32 (m, 2H), 7.13 (d, J = 1.7 Hz, 1H), 4.01- 3.35 (m, 8H), 2.74 (s, 3H). MS 273.3 (M + H). |
| 284 | (2-methyl-3-(1- methyl-1H- pyrazol-4- yl)quinolin-6- yl)(morpholino) methanone | 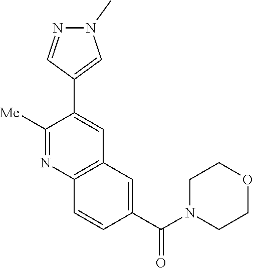 [SMILES: Cc1nc2ccc(C(=O)N3CCOCC3)cc2cc1-c1cnn(C)c1] | Production Ex. 18 | Production Ex. 431 | 1H NMR (500 MHz) δ 8.08-8.02 (m, 2H), 7.86 (d, J = 1.9 Hz, 1H), 7.73 (s, 1H), 7.66 (dd, J = 8.6, 1.9 Hz, 1H), 7.60 (s, 1H), 4.02 (s, 3H), 3.97-3.40 (m, 8H), 2.82 (s, 3H). MS 337.3 (M + H). |
| 285 | 4-([1,1′-biphenyl]- 2-yl)-6-(4- methylpiperazine-1- carbonyl)quinoline- 2-carbaldehyde | 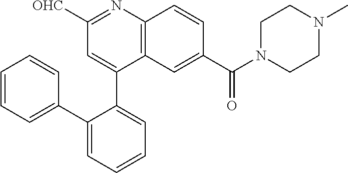 [SMILES: CN1CCN(C(=O)c2ccc3nc(C=O)cc(-c4ccccc4-c4ccccc4)c3c2)CC1] | Production Ex. 119 | Production Ex. 272 | 1H NMR (500 MHz) δ 10.19 (s, 1H), 8.22 (dd, J = 8.5, 0.7 Hz, 1H), 7.92 (s, 1H), 7.72-7.49 (m, 5H), 7.38 (dd, J = 7.6, 1.3 Hz, 1H), 7.08-6.98 (m, 5H), 4.31-3.33 (m, 4H), 3.12- 2.45 (m, 7H). MS 436.34 (M + H). |
| 286 | 4-([1,1′-biphenyl]- 2-yl)-2- formylquinoline-6- carboxamide | 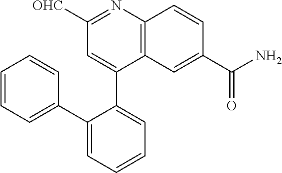 [SMILES: NC(=O)c1ccc2nc(C=O)cc(-c3ccccc3-c3ccccc3)c2c1] | Production Ex. 119 | Production Ex. 209 | MS 353.25 (M + H). |
| 287 | 4-(2-acetyl-1,2,3,4- tetrahydroisoquinolin- 6-yl)-6- (morpholine-4- carbonyl)quinoline- 2-carbaldehyde | 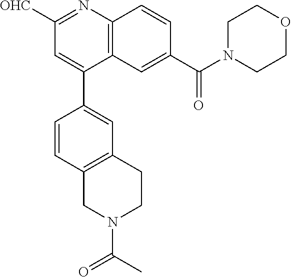 [SMILES: CC(=O)N1CCc2cc(-c3cc(C=O)nc4ccc(C(=O)N5CCOCC5)cc34)ccc2C1] | Production Ex. 119 | Production Ex. 372 | 1H NMR (500 MHz) 10.27 (s, 1H), 8.37 (d, J = 8.6 Hz, 1H), 8.05 (dd, J = 6.5, 1.8 Hz, 1H), 8.00 (s, 1H), 7.82 (dd, J = 8.9, 1.3 Hz, 1H), 7.37-7.28 (m, 3H), 4.86, 4.75 (each s, combined 2H), 3.95-3.32 (m, 10H), 3.01, 2.95 (each t, each J = 5.9 Hz, combined 2H), 2.25, 2.23 (each s, combined 3H). MS 444.34 (M + H). |
| 288 | tert-butyl 1-(4- ([1,1′-biphenyl]-2- yl)-2- formylquinoline-6- carbonyl)piperidine- 4-carboxylate | 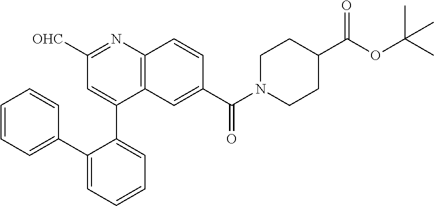 [SMILES: CC(C)(C)OC(=O)C1CCN(C(=O)c2ccc3nc(C=O)cc(-c4ccccc4-c4ccccc4)c3c2)CC1] | Production Ex. 119 | Production Ex. 273 | 1H NMR (500 MHz) δ 10.19 (s, 1H), 8.21 (d, J = 8.9 Hz, 1H), 7.89 (s, 1H), 7.72-7.64 (m, 2H), 7.62- 7.54 (m, 2H), 7.53-7.48 (m, 1H), 7.39-7.35 (m, 1H), 7.06-6.98 (m, 5H), 4.60-4.31 (m, 1H), 3.57- 3.32 (m, 1H), 3.17-2.87 (m, 2H), 2.53-2.44 (m, 1H), 2.09-1.51 (m, 4H), 1.47 (s, 9H). MS 521.39 (M + H). |
| 289 | tert-butyl ((4- ([1,1′-biphenyl]-2- yl)-2- formylquinolin-6- yl)methyl)(tetrahydro- 2H-pyran-4- yl)carbamate | 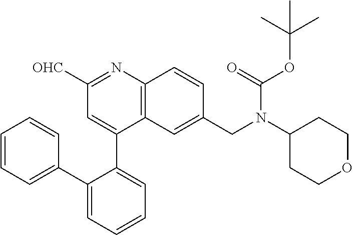 [SMILES: CC(C)(C)OC(=O)N(Cc1ccc2nc(C=O)cc(-c3ccccc3-c3ccccc3)c2c1)C1CCOCC1] | Production Ex. 119 | Production Ex. 414 | 1H NMR (500 MHz) δ 10.17 (s, 1H), 8.15 (d, J = 8.7 Hz, 1H), 7.83 (s, 1H), 7.63-7.55 (m, 3H), 7.53- 7.47 (m, 2H), 7.35 (dd, J = 7.6, 1.1 Hz, 1H), 7.07- 7.01 (m, 5H), 4.57-4.22 (m, 3H), 3.96-3.85 (m, 2H), 3.47-3.24 (m, 2H), 1.70-1.13 (m, 13H). MS 523.37 (M + H). |
| 290 | 6-(morpholine-4- carbonyl)-4-(N- tritylindazol-4- yl)quinoline-2- carbaldehyde | 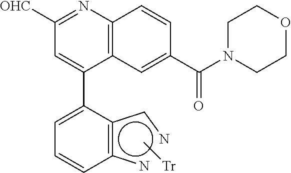 | Production Ex. 119 | Production Ex. 220 | MS 629.45 (M + H). |
| 291 | 4-(2-acetyl-1,2,3,4- tetrahydroisoquinol in-5-yl)-6- (morpholine-4- carbonyl)quinoline- 2-carbaldehyde | 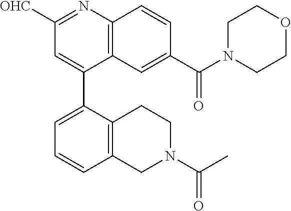 [SMILES: CC(=O)N1CCc2c(cccc2-c2cc(C=O)nc3ccc(C(=O)N4CCOCC4)cc23)C1] | Production Ex. 119 | Production Ex. 339 | MS 444.35 (M + H). |
| 292 | 6-(morpholine-4- carbonyl)-3- (naphthalen-1- yl)quinoline-2- carbaldehyde | 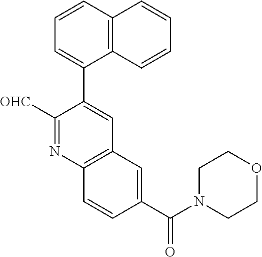 [SMILES: O=Cc1nc2ccc(C(=O)N3CCOCC3)cc2cc1-c1cccc2ccccc12] | Production Ex. 119 | Production Ex. 401 | 1H NMR (500 MHz) δ 10.04 (s, 1H), 8.47-8.43 (m, 1H), 8.31 (d, J = 0.9 Hz, 1H), 8.03-7.95 (m, 3H), 7.87 (dd, J = 8.7, 1.8 Hz, 1H), 7.61 (dd, J = 8.3, 6.9 Hz, 1H), 7.55-7.51 (m, 1H), 7.45 (dd, J = 6.9, 1.2 Hz, 1H), 7.42-7.34 (m, 2H), 3.97-3.44 (m, 8H). MS 397.31 (M + H). |
| 293 | 2-formyl-3- (naphthalen-1- yl)quinoline-6- carboxylic acid | 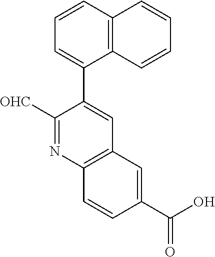 [SMILES: O=Cc1nc2ccc(C(=O)O)cc2cc1-c1cccc2ccccc12] | Production Ex. 119 | Production Ex. 422 | MS 328.3 (M + H). |
| 294 | 4-(3-(1-methyl-1H- pyrazol-4- yl)phenyl)-6- (morpholine-4- carbonyl)quinoline- 2-carbaldehyde | 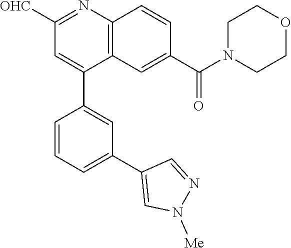 [SMILES: Cn1cc(-c2cccc(-c3cc(C=O)nc4ccc(C(=O)N5CCOCC5)cc34)c2)cn1] | Production Ex. 119 | Production Ex. 345 | 1H NMR (500 MHz) δ 10.29 (s, 1H), 8.38 (dd, J = 8.8, 0.6 Hz, 1H), 8.07 (dd, J = 1.9, 0.6 Hz, 1H), 8.06 (s, 1H), 7.85 (dd, J = 8.7, 1.8 Hz, 1H), 7.80 (d, J = 0.8 Hz, 1H), 7.68 (d, J = 0.8 Hz, 1H), 7.66-7.63 (m, 1H), 7.59-7.57 (m, 1H), 7.55 (t, J = 7.7 Hz, 1H), 7.35-7.32 (m, 1H), 3.97 (s, 3H), 3.87-3.34 (m, 8H). MS 427.32 (M + H). |
| 295 | tert-butyl 4-(2- formyl-6- (morpholine-4- carbonyl)quinolin- 4-yl)benzoate | 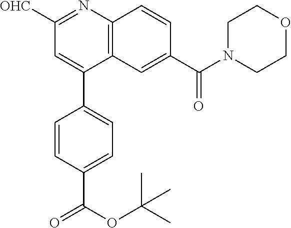 [SMILES: CC(C)(C)OC(=O)c1ccc(-c2cc(C=O)nc3ccc(C(=O)N4CCOCC4)cc23)cc1] | Production Ex. 119 | Production Ex. 346 | MS 447.31 (M + H). |
| 296 | tert-butyl 2-(2- formyl-6- (morpholine-4- carbonyl)quinolin- 4-yl)benzoate | 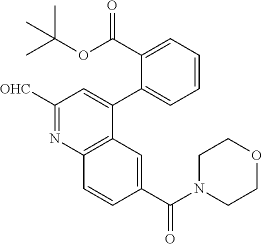 [SMILES: CC(C)(C)OC(=O)c1ccccc1-c1cc(C=O)nc2ccc(C(=O)N3CCOCC3)cc12] | Production Ex. 119 | Production Ex. 349 | MS 447.34 (M + H). |
| 297 | 4-(1-methyl-1H- pyrazol-4-yl)-6- (morpholine-4- carbonyl)quinoline- 2-carbaldehyde | 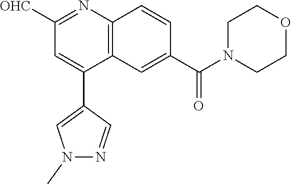 [SMILES: Cn1cc(-c2cc(C=O)nc3ccc(C(=O)N4CCOCC4)cc23)cn1] | Production Ex. 119 | Production Ex. 348 | MS 351.30 (M + H). |
| 298 | tert-butyl 3-(2- formyl-6- (morpholine-4- carbonyl)quinolin- 4-yl)benzoate | 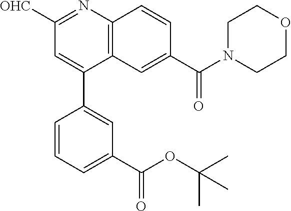 [SMILES: CC(C)(C)OC(=O)c1cccc(-c2cc(C=O)nc3ccc(C(=O)N4CCOCC4)cc23)c1] | Production Ex. 119 | Production Ex. 347 | MS 447.32 (M + H). |
| 299 | 6-(morpholine-4- carbonyl)-4-(3-(1- trityl-1H-pyrazol-4- yl)phenyl)quinoline- 2-carbaldehyde | 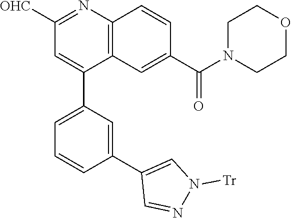 | Production Ex. 119 | Production Ex. 371 | 1H NMR (500 MHz) δ 10.27 (s, 1H), 8.36 (dd, J = 8.6, 0.6 Hz, 1H), 8.02 (s, 1H), 8.00 (dd, J = 1.8, 0.7 Hz, 1H), 7.97 (d, J = 0.8 Hz, 1H), 7.83 (dd, J = 8.6, 1.8 Hz, 1H), 7.69 (d, J = 0.8 Hz, 1H), 7.61-7.58 (m, 1H), 7.53-7.48 (m, 2H), 7.37-7.27 (m, 10H), 7.23-7.18 (m, 6H), 3.94- 3.27 (m, 8H). MS 655.39 (M + H). |
| 300 | 6-(morpholine-4- carbonyl)-4-(1- trityl-1H-pyrazol- 4-yl)quinoline-2- carbaldehyde | 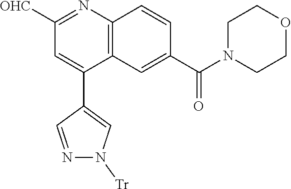 | Production Ex. 119 | Production Ex. 222 | 1H NMR (500 MHz) δ 10.21 (s, 1H), 8.31 (dd, J = 8.6, 0.6 Hz, 1H), 8.24 (dd, J = 1.9, 0.6 Hz, 1H), 8.02 (d, J = 0.8 Hz, 1H), 7.97 (s, 1H), 7.82 (dd, J = 8.6, 1.8 Hz, 1H), 7.78 (d, J = 0.8 Hz, 1H), 7.41-7.34 (m, 9H), 7.25-7.19 (m, 6H), 4.00-3.31 (m, 8H). MS 579.35 (M + H). |
| 301 | 6-(morpholine-4- carbonyl)-4- (pyridin-4- yl)quinoline-2- carbaldehyde | 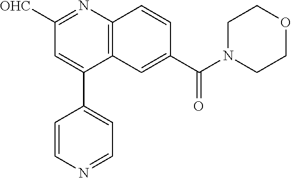 [SMILES: O=Cc1cc(-c2ccncc2)c2cc(C(=O)N3CCOCC3)ccc2n1] | Production Ex. 119 | Production Ex. 354 | 1H NMR (500 MHz) δ 10.27 (s, 1H), 8.86-8.83 (m, 2H), 8.41 (d, J = 8.7 Hz, 1H), 8.02 (s, 1H), 7.95 (d, J = 1.8 Hz, 1H), 7.87 (dd, J = 8.7, 1.8 Hz, 1H), 7.48-7.45 (m, 2H), 3.96- 3.30 (m, 8H). MS 366.22 (M + H). |
| 302 | 4-(1-methyl-1H- pyrazol-4- yl)quinoline-2- carbaldehyde | 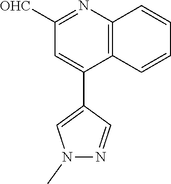 [SMILES: Cn1cc(-c2cc(C=O)nc3ccccc23)cn1] | Production Ex. 119 | Production Ex. 355 | 1H NMR (500 MHz) δ 10.23 (s, 1H), 8.30-8.26 (m, 2H), 7.95 (s, 1H), 7.87 (d, J = 0.8 Hz, 1H), 7.85- 7.81 (m, 1H), 7.79 (s, 1H), 7.71-7.67 (m, 1H), 4.06 (s, 3H). MS 238.03 (M + H). |
| 303 | 6-(morpholine-4- carbonyl)-4- (pyrimidin-5- yl)quinoline-2- carbaldehyde | 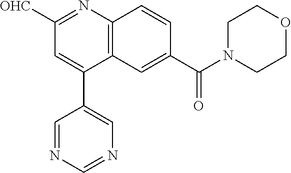 [SMILES: O=Cc1cc(-c2cncnc2)c2cc(C(=O)N3CCOCC3)ccc2n1] | Production Ex. 119 | Production Ex. 402 | 1H NMR (500 MHz) δ 10.28 (s, 1H), 9.43 (s, 1H), 8.95 (s, 2H), 8.44 (dd, J = 8.5, 0.7 Hz, 1H), 8.04 (s, 1H), 7.92-7.87 (m, 2H), 3.91-3.35 (m, 8H). MS 349.19 (M + H). |
| 304 | 4-(1-ethyl-1H- pyrazol-4-yl)-6- (morpholine-4- carbonyl)quinoline- 2-carbaldehyde | 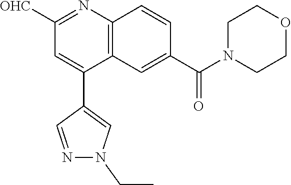 [SMILES: CCn1cc(-c2cc(C=O)nc3ccc(C(=O)N4CCOCC4)cc23)cn1] | Production Ex. 119 | Production Ex. 356 | 1H NMR (500 MHz) δ 10.22 (s, 1H), 8.35 (d, J = 1.8 Hz, 1H), 8.33 (d, J = 8.6 Hz, 1H), 8.00 (s, 1H), 7.88 (s, 1H), 7.87 (s, 1H), 7.82 (dd, J = 8.6, 1.8 Hz, 1H), 4.33 (q, J = 7.3 Hz, 2H), 3.98-3.37 (m, 8H), 1.61 (t, J = 7.3 Hz, 3H). MS 365.24 (M + H). |
| 305 | tert-butyl ((2- formyl-4-(1- methyl-1H- pyrazol-4- yl)quinolin-6- yl)methyl)(tetrahydro- 2H-pyran-4- yl)carbamate | 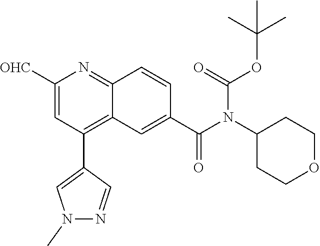 | Production Ex. 119 | Production Ex. 415 | 1H NMR (500 MHz) δ 10.21 (s, 1H), 8.22 (d, J = 8.7 Hz, 1H), 8.08 (s, 1H), 7.94 (s, 1H), 7.83 (s, 1H), 7.75 (s, 1H), 7.71 (dd, J = 8.7, 1.9 Hz, 1H), 4.71- 4.26 (m, 3H), 4.05 (s, 3H), 3.96-3.89 (m, 2H), 3.50- 3.22 (m, 2H), 1.83-1.14 (m, 13H). MS 451.35 (M + H). |
| 306 | 4-(1- acetylpiperidin-3- yl)-6-(morpholine-4- carbonyl)quinoline- 2-carbaldehyde | 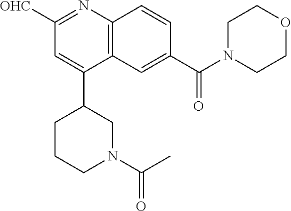 [SMILES: CC(=O)N1CCCC(c2cc(C=O)nc3ccc(C(=O)N4CCOCC4)cc23)C1] | Production Ex. 119 | Production Ex. 253 | 1H NMR (500 MHz) δ 10.21,10.20 (each s, combined 1H), 8.37-8.30 (m, 2H), 7.96, 7.95 (each s, combined 1H), 7.85-7.81 (m, 1H), 4.93-4.79 (m, 1H), 4.07-3.44 (m, 10H), 3.39-3.15 (m, 1H), 2.71- 2.62 (m, 1H), 2.25-2.13 (m, 4H), 2.06-1.68 (m, 3H). MS 396.36 (M + H). |
| 307 | 4-(morpholine-4- carbonyl)quinoline- 2-carbaldehyde | 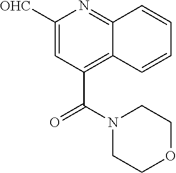 [SMILES: O=Cc1cc(C(=O)N2CCOCC2)c2ccccc2n1] | Production Ex. 119 | Production Ex. 404 | 1H NMR (500 MHz) δ 10.22 (s, 1H), 8.33-8.30 (m, 1H), 7.94-7.87 (m, 3H), 7.79-7.75 (m, 1H), 4.08-3.83 (m, 4H), 3.65- 3.48 (m, 2H), 3.29-3.10 (m, 2H). MS 271.18 (M + H). |
| 308 | 4-(2-acetyl-1,2,3,4- tetrahydroisoquinolin- 6-yl)quinoline- 2-carbaldehyde | 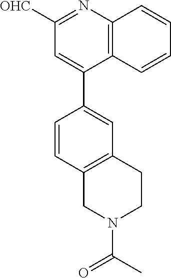 [SMILES: CC(=O)N1CCc2cc(-c3cc(C=O)nc4ccccc34)ccc2C1] | Production Ex. 119 | Production Ex. 254 | 1H NMR (500 MHz) δ 10.27 (s, 1H), 8.32 (d, J = 8.5 Hz, 1H), 8.01 (d, J = 8.5 Hz, 1H), 7.96 (s, 1H), 7.86-7.82 (m, 1H), 7.68- 7.64 (m, 1H), 7.39-7.29 (m, 3H), 4.86, 4.75 (each s, combined 2H), 3.91, 3.78 (each t, each J = 6.0 Hz, combined 2H), 3.02, 2.95 (each t, each J = 6.0 Hz, combined 2H), 2.25, 2.23 (each s, combined 3H). MS 331.21 (M + H). |
| 309 | 4-(1-methyl-1H- 1,2,4-triazol-3-yl)- 6-(morpholine-4- carbonyl)quinoline- 2-carbaldehyde | 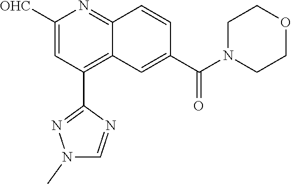 [SMILES: Cn1cnc(-c2cc(C=O)nc3ccc(C(=O)N4CCOCC4)cc23)n1] | Production Ex. 119 | Production Ex. 405 | 1H NMR (500 MHz) δ 10.27 (s, 1H), 9.46 (dd, J = 1.8, 0.6 Hz, 1H), 8.78 (s, 1H), 8.35 (dd, J = 8.7, 0.7 Hz, 1H), 8.25 (s, 1H), 7.89 (dd, J = 8.6, 1.9 Hz, 1H), 4.11 (s, 3H), 3.98-3.45 (m, 8H). MS 352.24 (M + H). |
| 310 | 4-(1-methyl-1H- imidazol-2-yl)-6- (morpholine-4- carbonyl)quinoline- 2-carbaldehyde | 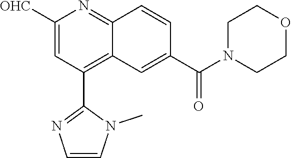 [SMILES: Cn1ccnc1-c1cc(C=O)nc2ccc(C(=O)N3CCOCC3)cc12] | Production Ex. 119 | Production Ex. 406 | 1H NMR (500 MHz) δ 10.27 (s, 1H), 8.42 (d, J = 1.8 Hz, 1H), 8.36 (d, J = 8.6 Hz, 1H), 8.06 (s, 1H), 7.89 (dd, J = 8.6, 1.9 Hz, 1H), 7.32-7.30 (m, 1H), 7.17 (d, J = 1.2 Hz, 1H), 3.93-3.43 (m, 11H). MS 351.26 (M + H). |
| 311 | 6-(morpholine-4- carbonyl)-4- (pyrimidin-2- yl)quinoline-2- carbaldehyde | 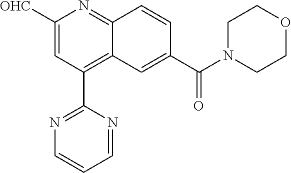 [SMILES: O=Cc1cc(-c2ncccn2)c2cc(C(=O)N3CCOCC3)ccc2n1] | Production Ex. 119 | Production Ex. 407 | 1H NMR (500 MHz) δ 10.30 (s, 1H), 9.08-9.05 (m, 1H), 9.01 (d, J = 4.8 Hz, 2H), 8.72 (s, 1H), 8.39 (d, J = 8.6 Hz, 1H), 7.88 (dd, J = 8.7, 1.5 Hz, 1H), 7.50-7.30 (m, 1H), 3.95- 3.47 (m, 8H). MS 349.18 (M + H). |
| 312 | 4-(4-(oxetan-3- yl)piperazine-1- carbonyl)quinoline- 2-carbaldehyde | 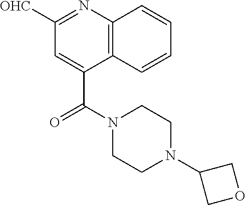 [SMILES: O=Cc1cc(C(=O)N2CCN(C3COC3)CC2)c2ccccc2n1] | Production Ex. 119 | Production Ex. 408 | 1H NMR (500 MHz) 8 10.23 (s, 1H), 8.31 (d, J = 8.5 Hz, 1H), 7.94-7.86 (m, 3H), 7.79-7.74 (m, 1H), 4.70-4.64 (m, 2H), 4.61-4.57 (m, 2H), 4.03- 3.98 (m, 2H), 3.58-3.51 (m, 1H), 3.31-3.17 (m, 2H), 2.53-2.49 (m, 2H), 2.21 (m, 2H). MS 326.21 (M + H). |
| 313 | 6-(morpholine-4- carbonyl)-4-(N- ((2- (trimethylsilyl)ethoxy) methyl)-1,2,4- triazol-3- yl)quinoline-2- carbaldehyde | 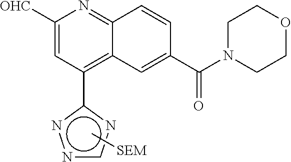 | Production Ex. 119 | Production Ex. 409 | MS 468.25 (M + H). |
| 314 | 4-(1-((2- (trimethylsilyl)ethoxy) methyl)-1H- pyrazol-4- yl)quinoline-2- carbaldehyde | 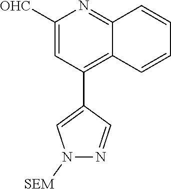 [SMILES: C[Si](C)(C)CCOCn1cc(-c2cc(C=O)nc3ccccc23)cn1] | Production Ex. 119 | Production Ex. 256 | 1H NMR (500 MHz) δ 10.25 (s, 1H), 8.38-8.27 (m, 1H), 8.27-8.25 (m, 1H), 8.02-7.98 (m, 2H), 7.93 (s, 1H), 7.88-7.83 (m, 1H), 7.74-7.69 (m, 1H), 5.57 (s, 2H), 3.76- 3.63 (m, 2H), 1.01-0.91 (m, 2H), 0.02 (s, 9H). MS 354.20 (M + H). |
| 315 | 6-(morpholine-4- carbonyl)-4- (pyridazin-3- yl)quinoline-2- carbaldehyde | 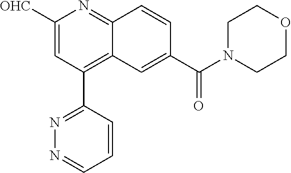 [SMILES: O=Cc1cc(-c2cccnn2)c2cc(C(=O)N3CCOCC3)ccc2n1] | Production Ex. 119 | Production Ex. 410 | 1H NMR (500 MHz) δ 10.28 (s, 1H), 9.39 (dd, J = 5.0, 1.7 Hz, 1H), 8.41 (d, J = 8.6 Hz, 1H), 8.36 (d, J = 1.8 Hz, 1H), 8.18 (s, 1H), 7.93-7.87 (m, 2H), 7.79 (dd, J = 8.4, 5.0 Hz, 1H), 3.90-3.46 (m, 8H). MS 349.17 (M + H). |
| 316 | 4-(1-methyl-1H- imidazol-4-yl)-6- (morpholine-4- carbonyl)quinoline- 2-carbaldehyde | 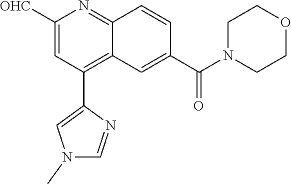 [SMILES: Cn1cnc(-c2cc(C=O)nc3ccc(C(=O)N4CCOCC4)cc23)c1] | Production Ex. 119 | Production Ex. 411 | 1H NMR (500 MHz) δ 10.23 (s, 1H), 9.20 (d, J = 1.8 Hz, 1H), 8.29 (d, J = 8.6 Hz, 1H), 8.19 (s, 1H), 7.85 (dd, J = 8.7, 1.9 Hz, 1H), 7.65 (s, 1H), 7.50 (d, J = 1.3 Hz, 1H), 3.94-3.49 (m, 11H). MS 351.19 (M + H). |
| 317 | 4-(2-acetyl-1,2,3,4- tetrahydroisoquinolin- 5-yl)quinoline- 2-carbaldehyde | 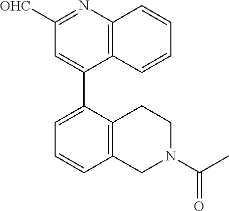 [SMILES: CC(=O)N1CCc2c(cccc2-c2cc(C=O)nc3ccccc23)C1] | Production Ex. 119 | Production Ex. 362 | 1H NMR (500 MHz) δ 10.29, 10.28 (each s, combined 1H), 8.36-8.31 (m, 1H), 7.93-7.82 (m, 2H), 7.66-7.58 (m, 1H), 7.58-7.51 (m, 1H), 7.41- 7.26 (m, 2H), 7.21-7.14 (m, 1H), 5.00-4.69 (m, 2H), 3.81-3.45 (m, 2H), 2.55-2.31 (m, 2H), 2.23, 2.11 (each s, combined 3H). MS 331.18 (M + H). |
| 318 | 4-(1-methyl-1H- pyrazol-4- yl)quinazolin-2- carbaldehyde | 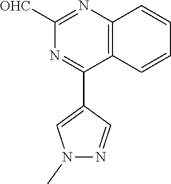 [SMILES: Cn1cc(-c2nc(C=O)nc3ccccc23)cn1] | Production Ex. 119 | Production Ex. 363 | 1H NMR (500 MHz) δ 10.26 (s, 1H), 8.46 (d, J = 8.5 Hz, 1H), 8.29 (s, 1H), 8.27 (d, J = 8.5 Hz, 1H), 8.22 (s, 1H), 8.05-8.00 (m, 1H), 7.85-7.81 (m, 1H), 4.07 (s, 3H). MS 239.08 (M + H). |
| 319 | 6-(morpholine-4- carbonyl)-4- (pyrazin-2- yl)quinoline-2- carbaldehyde | 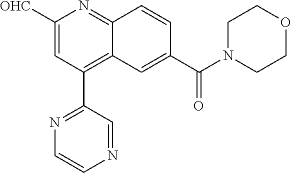 [SMILES: O=Cc1cc(-c2cnccn2)c2cc(C(=O)N3CCOCC3)ccc2n1] | Production Ex. 119 | Production Ex. 412 | 1H NMR (500 MHz) δ 10.29 (s, 1H), 9.02-9.00 (m, 1H), 8.85-8.77 (m, 2H), 8.43-8.37 (m, 2H), 8.22 (s, 1H), 7.91-7.87 (m, 1H), 3.92-3.42 (m, 8H). MS 349.1 (M + H). |
| 320 | 6-(morpholine-4- carbonyl)-5- (naphthalen-1- yl)quinoline-2- carbaldehyde | 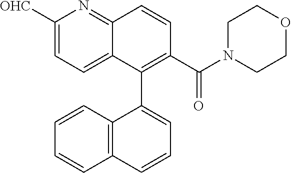 [SMILES: O=Cc1ccc2c(-c3cccc4ccccc34)c(C(=O)N3CCOCC3)ccc2n1] | Production Ex. 119 | Production Ex. 260 | 1H NMR (500 MHz) δ 10.26 (s, 1H), 8.42 (d, J = 8.7 Hz, 1H), 8.05-7.98 (m, 2H), 7.89-7.76 (m, 3H), 7.73-7.61 (m, 2H), 7.54-7.47 (m, 1H), 7.41- 7.21 (m, 1H), 7.05 (d, J = 8.5 Hz, 1H), 3.65-2.71 (m, 8H). MS 397.1 (M + H). |
| 321 | 4-(2-(1-methyl-1H- pyrazol-4- yl)phenyl)-6- (morpholine-4- carbonyl)quinoline- 2-carbaldehyde | 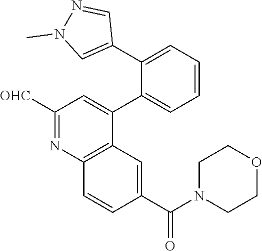 [SMILES: Cn1cc(-c2ccccc2-c2cc(C=O)nc3ccc(C(=O)N4CCOCC4)cc23)cn1] | Production Ex. 119 | Production Ex. 262 | 1H NMR (500 MHz) δ 10.26 (s, 1H), 8.32 (d, J = 8.6 Hz, 1H), 7.99 (s, 1H), 7.77 (dd, J = 8.7, 1.8 Hz, 1H), 7.61-7.51 (m, 3H), 7.45-7.38 (m, 1H), 7.31- 7.22 (m, 1H), 6.95 (s, 1H), 6.84 (s, 1H), 3.91-3.04 (m, 11H). MS 427.3 (M + H). |
| 322 | 6-(morpholine-4- carbonyl)-8- (naphthalen-1- yl)quinoline-2- carbaldehyde | 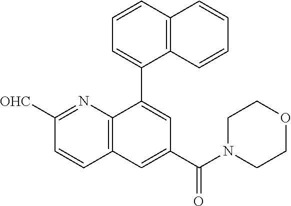 [SMILES: O=Cc1ccc2cc(C(=O)N3CCOCC3)cc(-c3cccc4ccccc34)c2n1] | Production Ex. 119 | Production Ex. 261 | 1H NMR (500 MHz) δ 9.76 (s, 1H), 8.44 (d, J = 8.5 Hz, 1H), 8.15-8.05 (m, 2H), 8.03-7.94 (m, 2H), 7.86 (d, J = 1.8 Hz, 1H), 7.73-7.59 (m, 1H), 7.57-7.43 (m, 2H), 7.39- 7.20 (m, 2H), 3.96-3.44 (m, 8H). MS 397.2 (M + H). |
| 323 | 3-(1-methyl-1H- pyrazol-4-yl)-6- (morpholine-4- carbonyl)quinoline- 2-carbaldehyde | 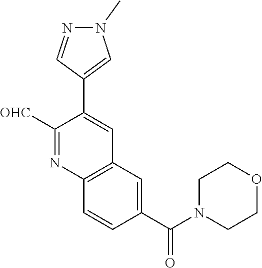 [SMILES: Cn1cc(-c2cc3cc(C(=O)N4CCOCC4)ccc3nc2C=O)cn1] | Production Ex. 119 | Production Ex. 284 | 1H NMR (500 MHz) δ 10.35 (s, 1H), 8.29 (d, J = 8.7 Hz, 1H), 8.26 (s, 1H), 7.94 (d, J = 1.8 Hz, 1H), 7.80 (s, 1H), 7.77 (dd, J = 8.7, 1.3 Hz, 1H), 7.75 (s, 1H), 4.03 (s, 3H), 3.95- 3.40 (m, 8H). MS 351.2 (M + H). |
| 324 | 5-(1-methyl-1H- pyrazol-4-yl)-6- (morpholine-4- carbonyl)quinoline- 2-carbaldehyde | 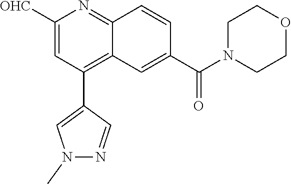 [SMILES: Cn1cc(-c2cc(C=O)nc3ccc(C(=O)N4CCOCC4)cc23)cn1] | Production Ex. 119 | Production Ex. 365 | 1H NMR (500 MHz) δ 10.25 (s, 1H), 8.54 (d, J = 8.7 Hz, 1H), 8.27 (d, J = 8.6 Hz, 1H), 8.04 (d, J = 8.7 Hz, 1H), 7.74 (d, J = 8.7 Hz, 1H), 7.67 (s, 1H), 7.65 (s, 1H), 4.04 (s, 3H), 3.79-3.59 (m, 3H), 3.49- 3.39 (m, 2H), 3.14-3.07 (m, 1H), 3.01-2.95 (m, 1H), 2.93-2.86 (m, 1H). MS 351.2 (M + H). |
| 325 | 3-(naphthalen-1- yl)-6-(N-((2- (trimethylsilyl)etho xy)methyl)tetrazol- 5-yl)quinoline-2- carbaldehyde | 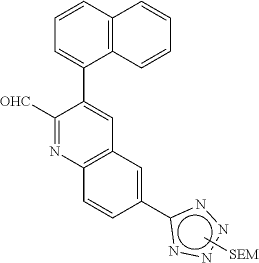 | Production Ex. 119 | Production Ex. 265 | MS 482.3 (M + H). |
| 326 | tert-butyl ((2- formyl-4-(2-(1- methyl-1H- pyrazol-4- yl)phenyl)quinolin-6- yl)methyl)(tetrahydro- 2H-pyran-4- yl)carbamate | 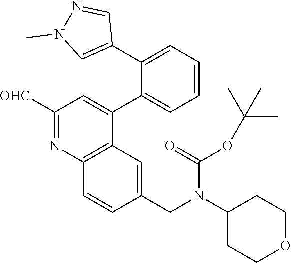 [SMILES: Cn1cc(-c2ccccc2-c2cc(C=O)nc3ccc(CN(C(=O)OC(C)(C)C)C4CCOCC4)cc23)cn1] | Production Ex. 119 | Production Ex. 366 | 1H NMR (500 MHz) δ 10.24 (s, 1H), 8.23 (d, J = 8.7 Hz, 1H), 7.91 (s, 1H), 7.68-7.63 (m, 1H), 7.58 (dd, J = 7.9, 1.3 Hz, 1H), 7.53-7.49 (m, 1H), 7.43- 7.36 (m, 2H), 7.23 (d, J = 7.7 Hz, 1H), 7.02 (s, 1H), 6.75 (s, 1H), 4.58-4.16 (m, 3H), 3.97-3.79 (m, 2H), 3.60 (s, 3H), 3.44- 3.18 (m, 2H), 1.68-1.04 (m, 13H). MS 527.4 (M + H). |
| 327 | 4- (morpholinomethyl) quinoline-2- carbaldehyde | 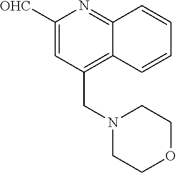 [SMILES: O=Cc1cc(CN2CCOCC2)c2ccccc2n1] | Production Ex. 119 | Production Ex. 266 | 1H NMR (500 MHz) δ 10.22 (s, 1H), 8.33 (d, J = 8.6 Hz, 1H), 8.27 (d, J = 8.6 Hz, 1H), 8.05 (s, 1H), 7.85-7.80 (m, 1H), 7.74- 7.69 (m, 1H), 3.98 (s, 2H), 3.75-3.71 (m, 4H), 2.56- 2.53 (m, 4H). MS 257.12 (M + H). |
| 328 | 4-(2-acetyl-1,2,3,4- tetrahydroisoquinol in-7-yl)-6- (morpholine-4- carbonyl)quinoline- 2-carbaldehyde | 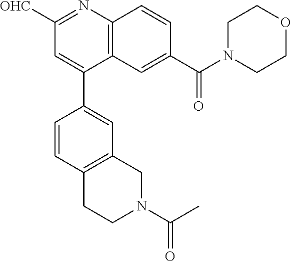 [SMILES: CC(=O)N1CCc2ccc(-c3cc(C=O)nc4ccc(C(=O)N5CCOCC5)cc34)cc2C1] | Production Ex. 119 | Production Ex. 367 | 1H NMR (500 MHz) δ 10.28-10.24 (m, 1H), 8.40- 8.34 (m, 1H), 8.09-8.03 (m, 1H), 8.03-7.96 (m, 1H), 7.83 (dd, J = 8.7, 1.8 Hz, 1H), 7.62-7.45 (m, 1H), 7.44-7.23 (m, 2H), 4.83, 4.73 (each s, combined 2H), 3.99-3.36 (m, 10H), 3.04, 2.98 (each t, each J = 5.9 Hz, combined 2H), 2.23 (s, 3H). MS 444.3 (M + H). |
| 329 | 5-(2-acetyl-1,2,3,4- tetrahydroisoquinolin- 6-yl)-6- (morpholine-4- carbonyl)quinoline- 2-carbaldehyde | 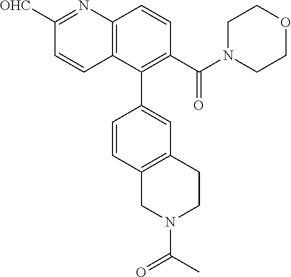 [SMILES: CC(=O)N1CCc2cc(-c3c(C(=O)N4CCOCC4)ccc4nc(C=O)ccc34)ccc2C1] | Production Ex. 119 | Production Ex. 368 | MS 444.3 (M + H). |
| 330 | 5-(2-acetyl-1,2,3,4- tetrahydroisoquinolin- 7-yl)-6- (morpholine-4- carbonyl)quinoline- 2-carbaldehyde | 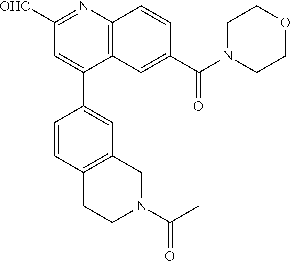 [SMILES: CC(=O)N1CCc2ccc(-c3cc(C=O)nc4ccc(C(=O)N5CCOCC5)cc34)cc2C1] | Production Ex. 119 | Production Ex. 369 | MS 444.3 (M + H). |
| 331 | 4-((3- oxomorpholino) methyl)quinoline-2- carbaldehyde | 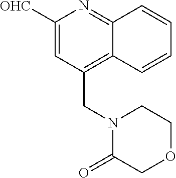 [SMILES: O=Cc1cc(CN2CCOCC2=O)c2ccccc2n1] | Production Ex. 119 | Production Ex. 267 | 1H NMR (500 MHz) δ 10.21 (s, 1H), 8.30 (d, J = 8.4 Hz, 1H), 8.19 (d, J = 8.4 Hz, 1H), 7.90-7.83 (m, 2H), 7.78-7.74 (m, 1H), 5.18 (s, 2H), 4.33 (s, 2H), 3.88 (t, J = 5.0 Hz, 2H), 3.31 (t, J = 5.0 Hz, 2H). MS 271.2 (M + H). |
| 332 | tert-butyl ((4-(2- acetyl-1,2,3,4- tetrahydroisoquinolin- 5-yl)-2- formylquinolin-6- yl)methyl)(tetrahydro- 2H-pyran-4- yl)carbamate | 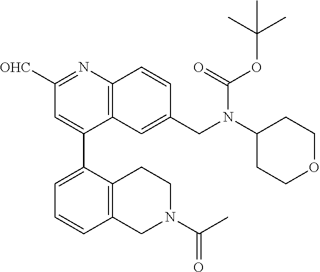 [SMILES: CC(=O)N1CCc2c(cccc2-c2cc(C=O)nc3ccc(CN(C(=O)OC(C)(C)C)C4CCOCC4)cc23)C1] | Production Ex. 119 | Production Ex. 370 | MS 544.4 (M + H). |
| 333 | tert-butyl ((2- formylquinolin-4- yl)methyl)(tetrahydro- 2H-pyran-4- yl)carbamate | 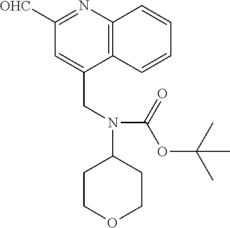 [SMILES: CC(C)(C)OC(=O)N(Cc1cc(C=O)nc2ccccc12)C1CCOCC1] | Production Ex. 119 | Production Ex. 421 | 1H NMR (500 MHz) δ 10.21 (s, 1H), 8.31 (d, J = 8.0 Hz, 1H), 8.04 (d, J = 8.0 Hz, 1H), 7.91 (s, 1H), 7.85 (t, J = 8.0 Hz, 1H), 7.74 (t, J = 8.0 Hz, 1H), 5.04-4.79 (m, 2H), 4.65- 4.43 (m, 1H), 3.97-3.92 (m, 2H), 3.53-3.36 (m, 2H), 1.76-1.16 (m, 13H). MS 371.3 (M + H). |
| 334 | tert-butyl ((4-(2- acetyl-1,2,3,4- tetrahydroisoquinolin- 6-yl)-2- formylquinolin-6- yl)methyl)(tetrahydro- 2H-pyran-4- yl)carbamate | 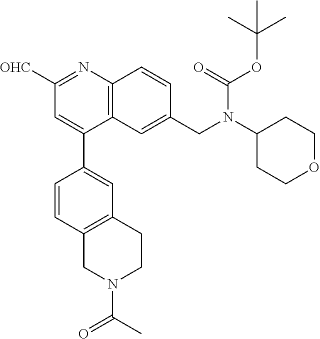 [SMILES: CC(=O)N1CCc2cc(-c3cc(C=O)nc4ccc(CN(C(=O)OC(C)(C)C)C5CCOCC5)cc34)ccc2C1] | Production Ex. 119 | Production Ex. 269 | 1H NMR (500 MHz) δ 10.25 (s, 1H), 8.27 (dd, J = 8.7, 2.2 Hz, 1H), 7.94 (s, 1H), 7.82-7.76 (m, 1H), 7.73 (d, J = 8.8 Hz, 1H), 7.46-7.11 (m, 3H), 4.86, 4.75 (each s, combined 2H), 4.65-4.24 (m, 3H), 3.96-3.87 (m, 3H), 3.78 (t, J = 5.9 Hz, 1H), 3.48-3.26 (m, 2H), 3.05-2.87 (m, 2H), 2.26, 2.24 (each s, combined 3H), 1.81-1.04 (m, 13H). MS 544.4 (M + H). |
| 335 | tert-butyl ((2- formyl-4-(3-(1- methyl-1H- pyrazol-4- yl)phenyl)quinolin-6- yl)methyl)(tetrahydro- 2H-pyran-4- yl)carbamate | 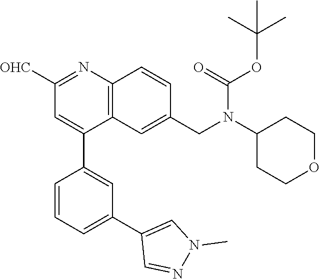 [SMILES: Cn1cc(-c2cccc(-c3cc(C=O)nc4ccc(CN(C(=O)OC(C)(C)C)C5CCOCC5)cc34)c2)cn1] | Production Ex. 119 | Production Ex. 270 | MS 527.4 (M + H). |
| 336 | tert-butyl ((4-(2- acetyl-1,2,3,4- tetrahydroisoquinolin- 7-yl)-2- formylquinolin-6- yl)methyl)(tetrahydro- 2H-pyran-4- yl)carbamate | 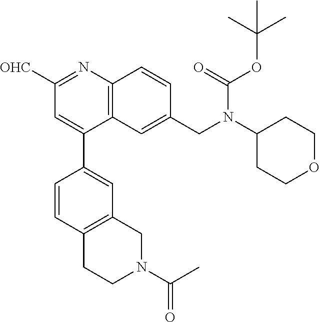 [SMILES: CC(=O)N1CCc2ccc(-c3cc(C=O)nc4ccc(CN(C(=O)OC(C)(C)C)C5CCOCC5)cc34)cc2C1] | Production Ex. 119 | Production Ex. 271 | 1H NMR (500 MHz) δ 10.25 (s, 1H), 8.33-8.23 (m, 1H), 7.97-7.91 (m, 1H), 7.83-7.71 (m, 2H), 7.39-6.58 (m, 3H), 4.83, 4.73 (each s, combined 2H), 4.65-4.22 (m, 3H), 3.97-3.87 (m, 3H), 3.79 (t, J = 5.9 Hz, 1H), 3.49-3.25 (m, 2H), 3.04, 2.97 (each t, each J = 6.0 Hz, combined 2H), 2.24, 2.23 (each s, combined 3H), 1.78-1.01 (m, 13H). MS 544.4 (M + H). |
| 337 | tert-butyl 6-(2- methyl-6- (morpholine-4- carbonyl)quinolin- 4-yl)-3,4- dihydroisoquinolin e-2(1H)- carboxylate | 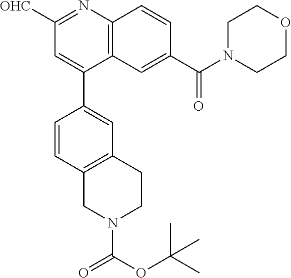 | Production Ex. 98 | Production Ex. 34 CAS No. 893566-72- 8 (Reagent Supplier 5) | 1H NMR (500 MHz) δ 8.10 (d, J = 8.9 Hz, 1H), 7.94 (s, 1H), 7.68 (dd, J = 8.6, 1.9 Hz, 1H), 7.31- 7.21 (m, 4H), 4.68 (s, 2H), 3.90-3.28 (m, 10H), 2.94- 2.89 (m, 2H), 2.79 (s, 3H), 1.53 (s, 9H). MS 488.41 (M + H). |
| 338 | ethyl 4-(1H- indazol-4-yl)-2- methylquinoline-6- carboxylate | 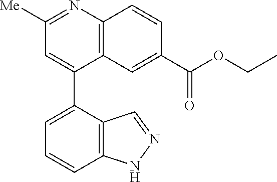 [SMILES: CCOC(=O)c1ccc2nc(C)cc(-c3cccc4[nH]ncc34)c2c1] | Production Ex. 98 | Production Ex. 66 CAS No. 1023595- 17- 0 (Reagent Supplier 4) | 1H NMR (500 MHz) δ 10.30 (s, 1H), 8.53 (d, J = 1.9 Hz, 1H), 8.31 (dd, J = 8.8, 1.9 Hz, 1H), 8.16 (d, J = 8.7 Hz, 1H), 7.79 (d, J = 1.1 Hz, 1H), 7.68-7.63 (m, 1H), 7.57 (dd, J = 8.4, 7.0 Hz, 1H), 7.46 (s, 1H), 7.29 (dd, J = 7.0, 0.8 Hz, 1H), 4.33 (q, J = 7.1 Hz, 2H), 2.84 (s, 3H), 1.33 (t, J = 7.1 Hz, 3H). MS 332.20 (M + H). |
| 339 | 1-(5-(2-methyl-6- (morpholine-4- carbonyl)quinolin- 4-yl)-3,4- dihydroisoquinolin- 2(1H)-yl)ethan-1- one | 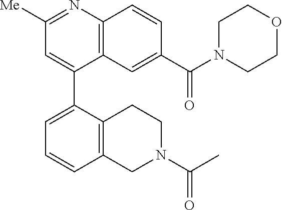 [SMILES: CC(=O)N1CCc2c(cccc2-c2cc(C)nc3ccc(C(=O)N4CCOCC4)cc23)C1] | Production Ex. 98 | Production Ex. 34 CAS No. 937591-82-7 (Reagent Supplier 2) | MS 430.39 (M + H). |
| 340 | ethyl 2-methyl-3- (naphthalen-1- yl)quinoline-6- carboxylate | 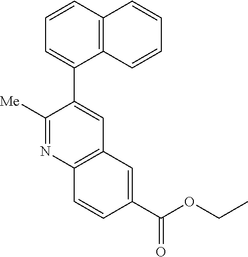 [SMILES: CCOC(=O)c1ccc2nc(C)c(-c3cccc4ccccc34)cc2c1] | Production Ex. 98 | Production Ex. 223 | 1H NMR (500 MHz) δ 8.57 (d, J = 1.9 Hz, 1H), 8.34 (dd, J = 8.8, 1.9 Hz, 1H), 8.16-8.13 (m, 1H), 8.12 (s, 1H), 7.97-7.94 (m, 2H), 7.58 (dd, J = 8.3, 7.0 Hz, 1H), 7.54-7.49 (m, 1H), 7.43-7.35 (m, 3H), 4.45 (q, J = 7.1 Hz, 2H), 2.45 (s, 3H), 1.44 (t, J = 7.1 Hz, 3H). MS 342.24 (M + H). |
| 341 | methyl 2-methyl-3- (naphthalen-1- yl)quinoline-6- carboxylate | 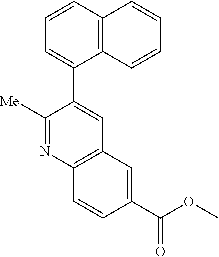 [SMILES: COC(=O)c1ccc2nc(C)c(-c3cccc4ccccc34)cc2c1] | Production Ex. 98 | Production Ex. 259 | 1H NMR (500 MHz) δ 8.59-8.57 (m, 1H), 8.36- 8.32 (m, 1H), 8.16 (d, J = 8.8 Hz, 1H), 8.13 (s, 1H), 7.97 (dd, J = 7.9, 1.7 Hz, 2H), 7.62-7.57 (m, 1H), 7.55-7.51 (m, 1H), 7.44- 7.37 (m, 3H), 4.00 (s, 3H), 2.46 (s, 3H). MS 328.2 (M + H). |
| 342 | ethyl 4-([1,1′- biphenyl]-3-yl)-2- methylquinoline-6- carboxylate | 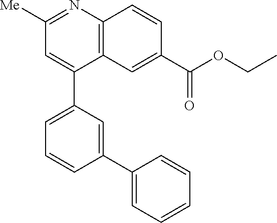 [SMILES: CCOC(=O)c1ccc2nc(C)cc(-c3cccc(-c4ccccc4)c3)c2c1] | Production Ex. 98 | Production Ex. 66 | 1H NMR (400 MHz) δ 8.74 (d, J = 1.9 Hz, 1H), 8.30 (dd, J = 9.0, 1.9 Hz, 1H), 8.13 (d, J = 8.7 Hz, 1H), 7.77-7.72 (m, 2H), 7.68-7.60 (m, 3H), 7.52- 7.44 (m, 3H), 7.41-7.35 (m, 2H), 4.38 (q, J = 7.2 Hz, 2H), 2.82 (s, 3H), 1.37 (t, J = 7.1 Hz, 3H). MS 368.31 (M + H). |
| 343 | ethyl 2-methyl-4- (naphthalen-1- yl)quinoline-6- carboxylate | 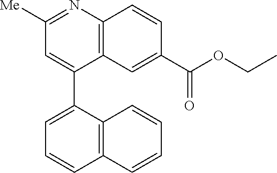 [SMILES: CCOC(=O)c1ccc2nc(C)cc(-c3cccc4ccccc34)c2c1] | Production Ex. 98 | Production Ex. 66 | 1H NMR (400 MHz) δ 8.28 (dd, J = 8.7, 1.8 Hz, 1H), 8.17 (d, J = 1.9 Hz, 1H), 8.15 (d, J = 9.1 Hz, 1H), 8.03-7.95 (m, 2H), 7.62 (dd, J = 8.3, 6.9 Hz, 1H), 7.54-7.44 (m, 2H), 7.39-7.32 (m, 3H), 4.27 (q, J = 7.2 Hz, 2H), 2.83 (s, 3H), 1.27 (t, J = 7.3 Hz, 3H). MS 342.14 (M + H). |
| 344 | 6-(2-(((tert- butyldimethylsilyl) oxy)methyl)-6- (morpholine-4- carbonyl)quinolin- 4-yl)-3,4- dihydroisoquinolin- 1(2H)-one | 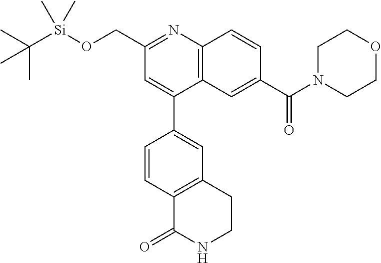 [SMILES: CC(C)(C)[Si](C)(C)OCc1cc(-c2ccc3c(c2)CCNC3=O)c2cc(C(=O)N3CCOCC3)ccc2n1] | Production Ex. 98 | Production Ex. 108 CAS No. 376584-30- 4 (Reagent Supplier 3) | 1H NMR (500 MHz) δ 8.25 (d, J = 7.9 Hz, 1H), 8.13 (dd, J = 8.6, 0.6 Hz, 1H), 7.91 (dd, J = 1.9, 0.6 Hz, 1H), 7.72 (dd, J = 8.6, 1.9 Hz, 1H), 7.68 (s, 1H), 7.48 (dd, J = 7.9, 1.8 Hz, 1H), 7.39-7.35 (m, 1H), 6.58 (s, 1H), 5.05 (s, 2H), 3.90-3.35 (m, 10H), 3.12 (t, J = 6.5 Hz, 2H), 0.97 (s, 9H), 0.16 (s, 6H). MS 532.51 (M + H). |
| 345 | (2-methyl-4-(3-(1- methyl-1H- pyrazol-4- yl)phenyl)quinolin-6- yl)(morpholino) methanone | 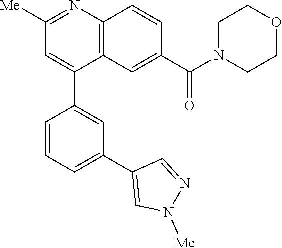 [SMILES: Cc1cc(-c2cccc(-c3cnn(C)c3)c2)c2cc(C(=O)N3CCOCC3)ccc2n1] | Production Ex. 98 | Production Ex. 34 CAS No. 1534350- 44- 1 (Reagent Supplier 16) | 1H NMR (500 MHz) δ 8.16-8.09 (m, 1H), 7.95- 7.94 (m, 1H), 7.79 (d, J = 0.8 Hz, 1H), 7.71 (dd, J = 8.6, 1.9 Hz, 1H), 7.67 (d, J = 0.8 Hz, 1H), 7.62-7.59 (m, 1H), 7.55-7.54 (m, 1H), 7.53-7.49 (m, 1H), 7.33-7.30 (m, 2H), 3.96 (s, 3H), 3.89-3.29 (m, 8H), 2.81 (s, 3H). MS 413.33 (M + H). |
| 346 | tert-butyl 4-(2- methyl-6- (morpholine-4- carbonyl)quinolin- 4-yl)benzoate | 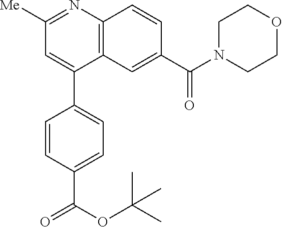 [SMILES: Cc1cc(-c2ccc(C(=O)OC(C)(C)C)cc2)c2cc(C(=O)N3CCOCC3)ccc2n1] | Production Ex. 98 | Production Ex. 34 CAS No. 850568-54- 6 (Reagent Supplier 5) | MS 433.34 (M + H). |
| 347 | tert-butyl 3-(2- methyl-6- (morpholine-4- carbonyl)quinolin- 4-yl)benzoate | 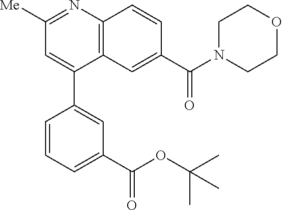 [SMILES: Cc1cc(-c2cccc(C(=O)OC(C)(C)C)c2)c2cc(C(=O)N3CCOCC3)ccc2n1] | Production Ex. 98 | Production Ex. 34 CAS No. 220210-56- 0 (Reagent Supplier 15) | MS 433.37 (M + H). |
| 348 | (2-methyl-4-(1- methyl-1H- pyrazol-4- yl)quinolin-6- yl)(morpholino) methanone | 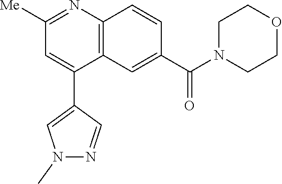 [SMILES: Cc1cc(-c2cnn(C)c2)c2cc(C(=O)N3CCOCC3)ccc2n1] | Production Ex. 98 | Production Ex. 34 CAS No. 847818-55- 7 (Reagent Supplier 16) | MS 337.25 (M + H). |
| 349 | tert-butyl 2-(2- methyl-6- (morpholine-4- carbonyl)quinolin- 4-yl)benzoate | 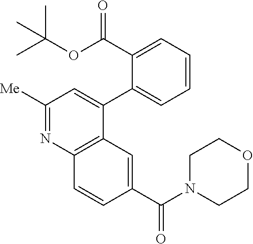 [SMILES: Cc1cc(-c2ccccc2C(=O)OC(C)(C)C)c2cc(C(=O)N3CCOCC3)ccc2n1] | Production Ex. 98 | Production Ex. 34 CAS No. 956229-69- 9 (Reagent Supplier 3) | MS 433.38 (M + H). |
| 350 | 4-(2-methyl- 1,2,3,4- tetrahydroisoquinolin- 6-yl)-6- (morpholine-4- carbonyl)quinoline- 2-carbaldehyde | 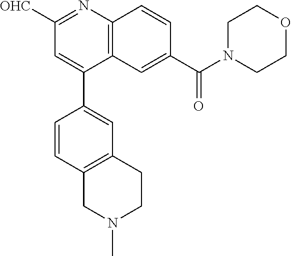 [SMILES: CN1CCc2cc(-c3cc(C=O)nc4ccc(C(=O)N5CCOCC5)cc34)ccc2C1] | Production Ex. 98 | Production Ex. 153 CAS No. 922718-57- 8 (Reagent Supplier 20) | MS 416.4 (M + H). |
| 351 | ethyl 4-([1,1′- biphenyl]-4-yl)-2- methylquinoline-6- carboxylate | 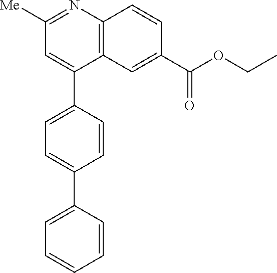 [SMILES: CCOC(=O)c1ccc2nc(C)cc(-c3ccc(-c4ccccc4)cc3)c2c1] | Production Ex. 98 | Production Ex. 66 | 1H NMR (400 MHz) δ 8.72 (d, J = 2.0 Hz, 1H), 8.30 (dd, J = 8.9, 2.0 Hz, 1H), 8.12 (d, J = 8.9 Hz, 7.72-7.68 (m, 2H), 7.60 (d, J = 8.2 Hz, 2H), 7.51 (t, J = 7.6 Hz, 2H), 7.42 (d, J = 7.4 Hz, 1H), 7.35 (s, 1H), 4.39 (q, J = 7.1 Hz, 2H), 2.82 (s, 3H), 1.39 (t, J = 7.1 Hz, 3H). MS 368.16 (M + H). |
| 352 | ethyl 2-methyl- [4,8′-biquinoline]- 6-carboxylate | 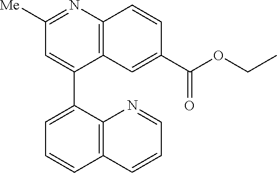 [SMILES: CCOC(=O)c1ccc2nc(C)cc(-c3cccc4cccnc34)c2c1] | Production Ex. 98 | Production Ex. 66 | 1H NMR (400 MHz) δ 8.81 (dd, J = 4.1, 1.8 Hz, 1H), 8.29 (dd, J = 8.3, 1.8 Hz, 1H), 8.24 (dd, J = 8.7, 1.9 Hz, 1H), 8.15-8.10 (m, 2H), 8.01 (dd, J = 6.7, 3.0 Hz, 1H), 7.73-7.66 (m, 2H), 7.45 (dd, J = 8.3, 4.2 Hz, 1H), 7.42 (s, 1H), 4.35-4.21 (m, 2H), 2.83 |
| | | | | | (s, 3H), 1.28 (t, J = 7.1 Hz, |
| | | | | | 3H). MS 343.16 (M + H). |
| 353 | ethyl 2-methyl-4- (naphthalen-2- yl)quinoline-6- carboxylate | 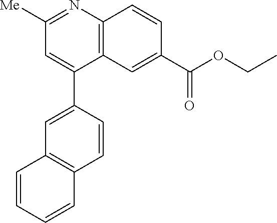 [SMILES: CCOC(=O)c1ccc2nc(C)cc(-c3ccc4ccccc4c3)c2c1] | Production Ex. 98 | Production Ex. 66 | 1H NMR (400 MHz) δ 8.67 (d, J = 1.9 Hz, 1H), 8.30 (dd, J = 8.7, 1.9 Hz, 1H), 8.14 (d, J = 9.0 Hz, 1H), 8.06-7.91 (m, 4H), 7.66-7.57 (m, 3H), 7.40 (s, 1H), 4.36 (q, J = 7.1 Hz, 2H), 2.83 (s, 3H), 1.34 (t, J = 7.2 Hz, 3H). MS 342.16 (M + H). |
| 354 | (2-methyl-4- (pyridin-4- yl)quinolin-6- yl)(morpholino) methanone | 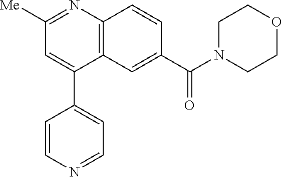 [SMILES: Cc1cc(-c2ccncc2)c2cc(C(=O)N3CCOCC3)ccc2n1] | Production Ex. 98 | Production Ex. 34 CAS No. 1692- 15- 5 (Reagent Supplier 4) | 1H NMR (500 MHz) δ 8.80 (d, J = 5.5 Hz, 2H), 8.15 (d, J = 8.6 Hz, 1H), 7.84 (d, J = 1.8 Hz, 1H), 7.72 (dd, J = 8.6, 1.8 Hz, 1H), 7.43 (d, J = 5.5 Hz, 2H), 7.29 (s, 1H), 3.95- 3.35 (m, 8H), 2.82 (s, 3H). MS 334.19 (M + H). |
| 355 | 2-methyl-4-(1- methyl-1H- pyrazol-4- yl)quinoline | 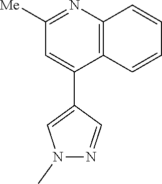 [SMILES: Cc1cc(-c2cnn(C)c2)c2ccccc2n1] | Production Ex. 98 | CAS No. 4295-06- 1 (Reagent Supplier 4) CAS No. 847818-55- 7 (Reagent Supplier 16) | 1H NMR (500 MHz) δ 8.13 (dd, J = 8.4, 1.5 Hz, 1H), 8.07 (dd, J = 8.4, 1.5 Hz, 1H), 7.81 (d, J = 0.8 Hz, 1H), 7.72-7.66 (m, 2H), 7.51-7.46 (m, 1H), 7.24 (s, 1H), 4.04 (s, 3H), 2.74 (s, 3H). MS 224.17 (M + H). |
| 356 | (4-(1-ethyl-1H- pyrazol-4-yl)-2- methylquinolin-6- yl)(morpholino) methanone | 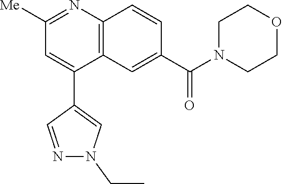 [SMILES: CCn1cc(-c2cc(C)nc3ccc(C(=O)N4CCOCC4)cc23)cn1] | Production Ex. 98 | Production Ex. 34 CAS No. 847818-56- 8 (Reagent Supplier 5) | 1H NMR (500 MHz) δ 8.23 (d, J = 1.8 Hz, 1H), 8.08 (d, J = 8.6 Hz, 1H), 7.81 (d, J = 0.8 Hz, 1H), 7.77 (d, J = 0.8 Hz, 1H), 7.68 (dd, J = 8.6, 1.8 Hz, 1H), 7.31 (s, 1H), 4.31 (q, J = 7.3 Hz, 2H), 4.02-3.29 (m, 8H), 2.76 (s, 3H), 1.60 (t, J = 7.3 Hz, 3H). MS 351.23 (M + H). |
| 357 | 2-methyl-4-(1- methyl-1H- pyrazol-4-yl)-N- (tetrahydro-2H- pyran-4- yl)quinoline-6- carboxamide | 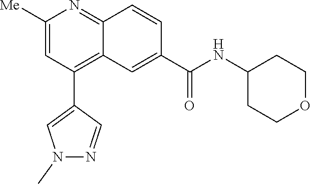 [SMILES: Cc1cc(-c2cnn(C)c2)c2cc(C(=O)NC3CCOCC3)ccc2n1] | Production Ex. 98 | Production Ex. 403 CAS No. 847818-55- 7 (Reagent Supplier 16) | 1H NMR (500 MHz) δ 8.66 (d, J = 2.0 Hz, 1H), 8.09 (d, J = 8.7 Hz, 1H), 7.94 (dd, J = 8.8, 2.0 Hz, 1H), 7.83 (d, J = 0.8 Hz, 1H), 7.80 (s, 1H), 7.32 (s, 1H), 6.08 (d, J = 7.8 Hz, 1H), 4.30-4.19 (m, 1H), 4.06 (s, 3H), 4.05-4.00 (m, 2H), 3.60-3.51 (m, 2H), 2.77 (s, 3H), 2.08- |
| | | | | | 2.01 (m, 2H), 1.67-1.55 |
| | | | | | (m, 2H). MS 351.25 |
| | | | | | (M + H). |
| 358 | tert-butyl 5-(2- methyl-6- (morpholine-4- carbonyl)quinolin- 4-yl)-3,6- dihydropyridine- 1(2H)-carboxylate | 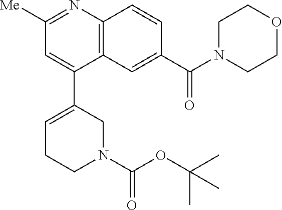 [SMILES: Cc1cc(C2=CCCN(C(=O)OC(C)(C)C)C2)c2cc(C(=O)N3CCOCC3)ccc2n1] | Production Ex. 98 | Production Ex. 34 CAS No. 885693-20- 9 (Reagent Supplier 4) | 1H NMR (500 MHz) δ 8.09 (d, J = 1.9 Hz, 1H), 8.06 (d, J = 8.6 Hz, 1H), 7.67 (dd, J = 8.6, 1.9 Hz, 1H), 7.19 (s, 1H), 6.01- 5.96 (m, 1H), 4.25-4.09 (m, 2H), 3.97-3.34 (m, 10H), 2.75 (s, 3H), 2.44- 2.37 (m, 2H), 1.50 (s, 9H). MS 438.51 (M + H). |
| 359 | tert-butyl 6-(2- methylquinolin-4- yl)-3,4- dihydroisoquinoline- 2(1H)- carboxylate | 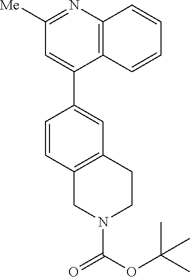 [SMILES: Cc1cc(-c2ccc3c(c2)CCN(C(=O)OC(C)(C)C)C3)c2ccccc2n1] | Production Ex. 98 | CAS No. 4295-06- 1 (Reagent Supplier 4) CAS No. 893566-72- 8 (Reagent Supplier 5) | 1H NMR (500 MHz) δ 8.08 (dd, J = 8.5, 1.3 Hz, 1H), 7.86 (dd, J = 8.5, 1.3 Hz, 1H), 7.69-7.64 (m, 1H), 7.44-7.40 (m, 1H), 7.33-7.29 (m, 1H), 7.27- 7.23 (m, 2H), 7.20 (s, 1H), 4.69 (s, 2H), 3.75-3.69 (m, 2H), 2.94-2.88 (m, 2H), 2.76 (s, 3H), 1.53 (s, 9H). MS 375.33 (M + H). |
| 360 | 2-methyl-4-(1H- pyrazol-4- yl)quinoline | 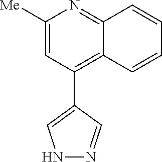 [SMILES: Cc1cc(-c2cn[nH]c2)c2ccccc2n1] | Production Ex. 98 | CAS No. 4295-06- 1 (Reagent Supplier 4) CAS No. 763120-58- 7 (Reagent Supplier 4) | MS 210.12 (M + H). |
| 361 | 4-(1-oxo-1,2,3,4- tetrahydroisoquinol in-6-yl)quinoline- 2-carbaldehyde | 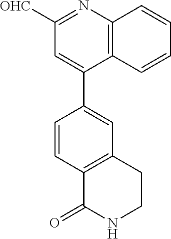 [SMILES: O=Cc1cc(-c2ccc3c(c2)CCNC3=O)c2ccccc2n1] | Production Ex. 98 | CAS No. 28615-67-0 (Reagent Supplier 20) CAS No. 376584-30- 4 (Reagent Supplier 3) | 1H NMR (500 MHz) δ 10.28 (s, 1H), 8.34 (d, J = 8.4 Hz, 1H), 8.25 (d, J = 8.4 Hz, 1H), 8.01-7.95 (m, 2H), 7.86 (t, J = 7.7 Hz, 1H), 7.68 (t, J = 7.7 Hz, 1H), 7.52 (d, J = 7.7 Hz, 1H), 7.41 (s, 1H), 7.15 (br s, 1H), 3.72-3.66 (m, 2H), 3.15-3.09 (m, 2H). MS 321.1 (M + H). |
| 362 | 1-(5-(2- methylquinolin-4- yl)-3,4- dihydroisoquinolin- 2(1H)-yl)ethan-1- one | 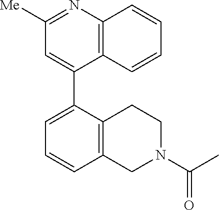 [SMILES: CC(=O)N1CCc2c(cccc2-c2cc(C)nc3ccccc23)C1] | Production Ex. 98 | CAS No. 50488-44- 3 (Reagent Supplier 3) CAS No. 937591-82- 7 (Reagent Supplier 2) | 1H NMR (500 MHz) δ 8.12-8.06 (m, 1H), 7.71- 7.65 (m, 1H), 7.42-7.24 (m, 4H), 7.20-7.14 (m, 2H), 4.95-4.68 (m, 2H), 3.69-3.46 (m, 2H), 2.79, 2.78 (each s, ??? 3H), 2.56-2.33 (m, 2H), 2.22, 2.10 (each s, combined 3H). MS 317.17 (M + H). |
| 363 | 2-methyl-4-(1- methyl-1H- pyrazol-4- yl)quinazoline | 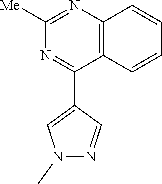 [SMILES: Cc1nc(-c2cnn(C)c2)c2ccccc2n1] | Production Ex. 98 | CAS No. 6484-24- 8 (Reagent Supplier 5) CAS No. 761446-44- 0 (Reagent Supplier 4) | 1H NMR (500 MHz) δ 8.30 (d, J = 8.3 Hz, 1H), 8.13 (s, 1H), 8.11 (s, 1H), 7.97 (d, J = 8.3 Hz, 1H), 7.88-7.84 (m, 1H), 7.60- 7.56 (m, 1H), 4.05 (s, 3H), 2.88 (s, 3H). MS 225.09 (M + H). |
| 364 | ethyl 2-methyl-3- (1-methyl-1H- pyrazol-4- yl)quinoline-6- carboxylate | 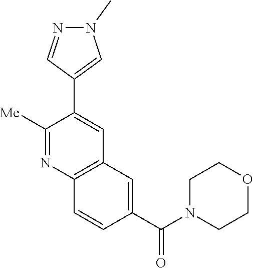 | Production Ex. 98 | Production Ex. 223 CAS No. 761446-44- 0 (Reagent Supplier 4) | 1H NMR (500 MHz) δ 8.55 (d, J = 1.9 Hz, 1H), 8.26 (dd, J = 8.8, 1.9 Hz, 1H), 8.11 (s, 1H), 8.05 (d, J = 8.8 Hz, 1H), 7.73 (s, 1H), 7.61 (s, 1H), 4.45 (q, J = 7.2 Hz, 2H), 4.03 (s, 3H), 2.83 (s, 3H), 1.45 (t, J = 7.1 Hz, 3H). MS 296.2 (M + H). |
| 365 | (2-methyl-5-(1- methyl-1H- pyrazol-4- yl)quinolin-6- yl)(morpholino) methanone | 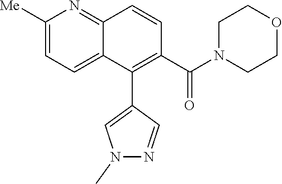 [SMILES: Cc1ccc2c(-c3cnn(C)c3)c(C(=O)N3CCOCC3)ccc2n1] | Production Ex. 98 | Production Ex. 257 CAS No. 761446-44- 0 (Reagent Supplier 4) | 1H NMR (500 MHz) δ 8.27 (d, J = 8.7 Hz, 1H), 8.03 (d, J = 8.6 Hz, 1H), 7.64 (s, 1H), 7.63 (s, 1H), 7.59 (d, J = 8.6 Hz, 1H), 7.31 (d, J = 8.7 Hz, 1H), 4.02 (s, 3H), 3.75-3.58 (m, 3H), 3.46-3.35 (m, 2H), 3.12-3.04 (m, 1H), 3.00-2.92 (m, 1H), 2.92- 2.85 (m, 1H), 2.76 (s, |
| | | | | | 3H). MS 337.2 (M + H). |
| 366 | tert-butyl ((2- methyl-4-(2-(1- methyl-1H- pyrazol-4- yl)phenyl)quinolin-6- yl)methyl)(tetrahydro- 2H-pyran-4- yl)carbamate | 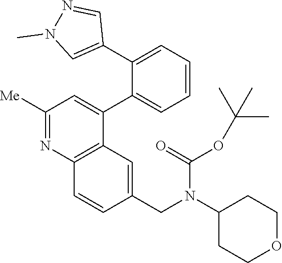 [SMILES: Cc1cc(-c2ccccc2-c2cnn(C)c2)c2cc(CN(C(=O)OC(C)(C)C)C3CCOCC3)ccc2n1] | Production Ex. 98 | Production Ex. 420 CAS No. 1638289- 93-6 (Synthetic Literature 11) | MS 513.4 (M + H). |
| 367 | 1-(7-(2-methyl-6- (morpholine-4- carbonyl)quinolin- 4-yl)-3,4- dihydroisoquinolin- 2(1H)-yl)ethan-1- one | 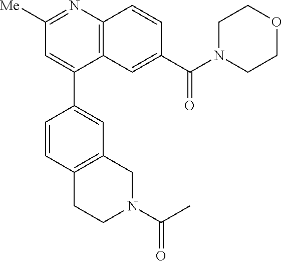 [SMILES: CC(=O)N1CCc2ccc(-c3cc(C)nc4ccc(C(=O)N5CCOCC5)cc34)cc2C1] | Production Ex. 98 | Production Ex. 34 CAS No. 937591-29- 2 (Reagent Supplier 9) | 1H NMR (500 MHz) δ 8.11 (dd, J = 8.6, 3.7 Hz, 1H), 7.93 (d, J = 12.2 Hz, 1H), 7.69 (d, J = 8.7 Hz, 1H), 7.62-7.19 (m, 4H), 4.82, 4.71 (each s, combined 2H), 3.94-3.34 (m, 10H), 3.07-2.92 (m, 2H), 2.79 (s, 3H), 2.22 (s, 3H). MS 430.3 (M + H). |
| 368 | 1-(6-(2-methyl-6- (morpholine-4- carbonyl)quinolin- 5-yl)-3,4- dihydroisoquinolin- 2(1H)-yl)ethan-1- one | 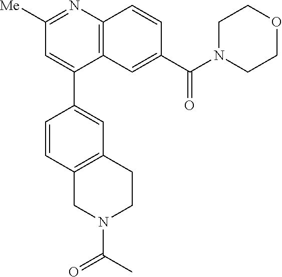 [SMILES: CC(=O)N1CCc2cc(-c3cc(C)nc4ccc(C(=O)N5CCOCC5)cc34)ccc2C1] | Production Ex. 98 | Production Ex. 257 CAS No. 1181691- 47- 3 (Reagent Supplier 9) | MS 430.3 (M + H). |
| 369 | 1-(7-(2-methyl-6- (morpholine-4- carbonyl)quinolin- 5-yl)-3,4- dihydroisoquinolin- 2(1H)-yl)ethan-1- one | 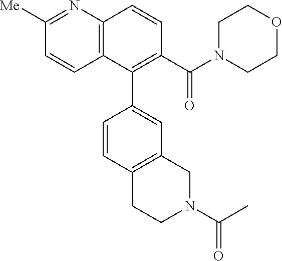 [SMILES: CC(=O)N1CCc2ccc(-c3c(C(=O)N4CCOCC4)ccc4nc(C)ccc34)cc2C1] | Production Ex. 98 | Production Ex. 257 CAS No. 937591-29- 2 (Reagent Supplier 9) | MS 430.3 (M + H). |
| 370 | tert-butyl ((4-(2- acetyl-1,2,3,4- tetrahydroisoquinolin- 5-yl)-2- methylquinolin-6- yl)methyl)(tetrahydro- 2H-pyran-4- yl)carbamate | 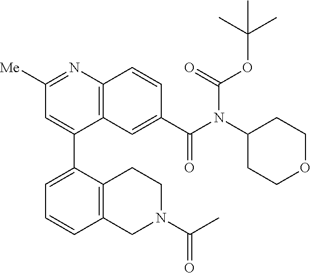 | Production Ex. 98 | Production Ex. 420 CAS No. 937591-82- 7 (Reagent Supplier 2) | MS 530.4 (M + H). |
| 371 | (2-methyl-4-(3-(1- trityl-1H-pyrazol-4- yl)phenyl)quinolin-6- yl)(morpholino) methanone | 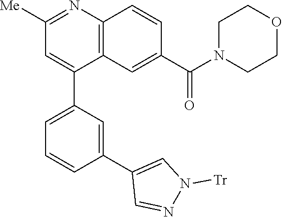 | Production Ex. 98 | Production Ex. 34 Product ion Ex. 237 | MS 641.41 (M + H). |
| 372 | 1-(6-(2-methyl-6- (morpholine-4- carbonyl)quinolin- 4-yl)-3,4- dihydroisoquinolin- 2(1H)-yl)ethan-1- one | 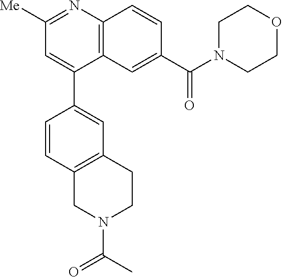 [SMILES: CC(=O)N1CCc2cc(-c3cc(C)nc4ccc(C(=O)N5CCOCC5)cc34)ccc2C1] | Production Ex. 48 | Production Ex. 211 | 1H NMR (500 MHz) δ 8.10 (d, J = 8.0 Hz, 1H), 7.92 (dd, J = 6.2, 1.8 Hz, 1H), 7.67 (dd, J = 8.6, 1.8 Hz, 1H), 7.32-7.20 (m, 4H), 4.83, 4.72 (each s, combined 2H), 3.93-3.34 (m, 10H), 2.98, 2.92 (each t, each J = 5.9 Hz, combined 2H), 2.78, 2.78 (each s, combined 3H), 2.23, 2.21 (each s, combined 3H). MS 430.32 (M + H). |
| 373 | 1-acetyl-2-((6- (morpholine-4- carbonyl)-4-(N- tritylindazol-4- yl)quinolin-2- yl)methylene ) indolin-3-one | 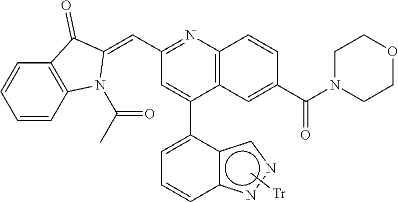 | Production Ex. 194 | Production Ex. 290 CAS No. 16800- 68- (Reagent Supplier 3) | MS 786.72 (M + H). |
| 374 | tert-butyl (Z)-4- ([1,1′-biphenyl]-3- yl)-2-((1-acetyl-3- oxoindolin-2- ylidene)methyl) quinoline-6- carboxylate | 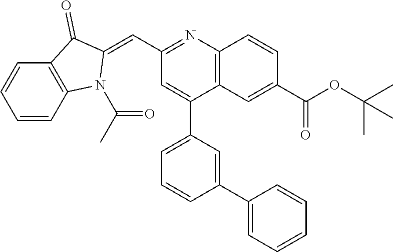 [SMILES: CC(=O)N1/C(=C\c2cc(-c3cccc(-c4ccccc4)c3)c3cc(C(=O)OC(C)(C)C)ccc3n2)C(=O)c2ccccc21] | Production Ex. 194 | Production Ex. 224 CAS No. 16800- 68- 3 (Reagent Supplier 3) | 1H NMR (400 MHz) δ 8.71 (d, J = 1.9 Hz, 1H), 8.28 (dd, J = 9.1, 1.9 Hz, 1H), 8.23-8.15 (m, 2H), 7.85 (dd, J = 7.9, 1.4 Hz, 1H), 7.80-7.74 (m, 2H), 7.70-7.62 (m, 5H), 7.56- 7.51 (m, 1H), 7.50-7.44 (m, 2H), 7.42-7.35 (m, 2H), 7.26 (t, J = 7.5 Hz, 1H), 2.21 (s, 3H), 1.57 (s, 9H). MS 567.15 (M + H) |
| 375 | tert-butyl (Z)-2- ((1-acetyl-3- oxoindolin-2- ylidene)methyl)-4- (naphthalen-1- yl)quinoline-6- carboxylate | 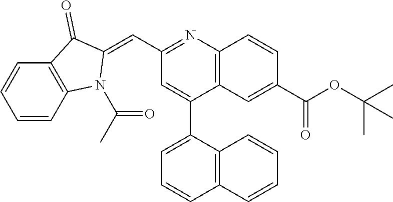 [SMILES: CC(=O)N1/C(=C\c2cc(-c3cccc4ccccc34)c3cc(C(=O)OC(C)(C)C)ccc3n2)C(=O)c2ccccc21] | Production Ex. 194 | Production Ex. 225 CAS No. 16800- 68-3 (Reagent Supplier 3) | 1H NMR (400 MHz) δ 8.27-8.15 (m, 4H), 8.03 (dd, J = 8.4, 1.2 Hz, 1H), 7.98 (d, J = 8.3 Hz, 1H), 7.87-7.83 (m, 1H), 7.70- 7.61 (m, 3H), 7.56-7.51 (m, 1H), 7.49 (dd, J = 6.9, 1.3 Hz, 1H), 7.40-7.34 (m, 3H), 7.29-7.23 (m, 1H), 2.28 (s, 3H), 1.48 (s, 9H). MS 541.19 (M + H). |
| 376 | tert-butyl (Z)-4- ([1,1′-biphenyl]-4- yl)-2-((1-acetyl-3- oxoindolin-2- ylidene)methyl) quinoline-6- carboxylate | 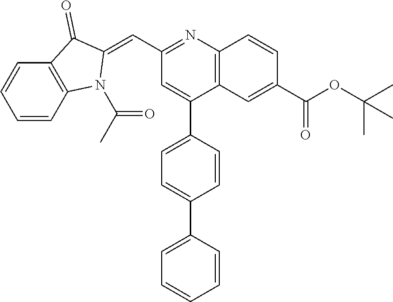 [SMILES: CC(=O)N1/C(=C\c2cc(-c3ccc(-c4ccccc4)cc3)c3cc(C(=O)OC(C)(C)C)ccc3n2)C(=O)c2ccccc21] | Production Ex. 194 | Production Ex. 226 CAS No. 16800- 68- (Reagent Supplier 3) | 1H NMR (400 MHz) δ 8.72 (d, J = 1.9 Hz, 1H), 8.27 (dd, J = 8.7, 1.8 Hz, 1H), 8.21 (d, J = 8.4 Hz, 1H), 8.18 (d, J = 8.8 Hz, 1H), 7.87-7.83 (m, 1H), 7.82-7.79 (m, 2H), 7.72- 7.61 (m, 6H), 7.54-7.48 (m, 2H), 7.45-7.40 (m, 1H), 7.37 (s, 1H), 7.29- 7.23 (m, 1H), 2.21 (s, 3H), 1.60 (s, 9H). MS 567.23 (M + H). |
| 377 | tert-butyl (Z)-2- ((1-acetyl-3- oxoindolin-2- ylidene)methyl)- [4,8′-biquinoline]- 6-carboxylate | 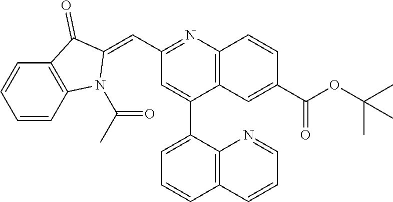 [SMILES: CC(=O)N1/C(=C\c2cc(-c3cccc4cccnc34)c3cc(C(=O)OC(C)(C)C)ccc3n2)C(=O)c2ccccc21] | Production Ex. 194 | Production Ex. 227 CAS No. 16800- 68- 3 (Reagent Supplier 3) | 1H NMR (400 MHz) δ 8.75 (dd, J = 4.1, 1.8 Hz, 1H), 8.23 (dd, J = 8.3, 1.8 Hz, 1H), 8.17-8.09 (m, 4H), 7.96 (dd, J = 7.6, 2.0 Hz, 1H), 7.77 (dd, J = 7.5, 1.3 Hz, 1H), 7.70-7.62 (m, 3H), 7.61-7.56 (m, 1H), 7.41 (dd, J = 8.3, 4.1 Hz, 1H), 7.33 (s, 1H), 7.18 (t, J = 7.4 Hz, 1H), 2.23 (s, 3H), 1.42 (s, 9H). MS 542.24 (M + H). |
| 378 | tert-butyl (Z)-2- ((1-acetyl-3- oxoindolin-2- ylidene )methyl)-4- (naphthalen-2- yl)quinoline-6- carboxylate | 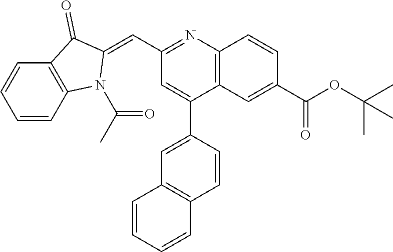 [SMILES: CC(=O)N1/C(=C\c2cc(-c3ccc4ccccc4c3)c3cc(C(=O)OC(C)(C)C)ccc3n2)C(=O)c2ccccc21] | Production Ex. 194 | Production Ex. 228 CAS No. 16800- 68- 3 (Reagent Supplier 3) | 1H NMR (400 MHz) 8 8.69 (d, J = 1.8 Hz, 1H), 8.28 (dd, J = 9.1, 1.8 Hz, 1H), 8.23-8.17 (m, 2H), 8.07-8.03 (m, 2H), 8.00- 7.93 (m, 2H), 7.85 (dd, J = 7.8, 1.4 Hz, 1H), 7.71- 7.57 (m, 5H), 7.38 (s, 1H), 7.26 (t, J = 7.5 Hz, 1H), 2.22 (s, 3H), 1.56 (s, 9H). MS 541.21 (M + H). |
| 379 | tert-butyl (Z)-((4- ([1,1′-biphenyl]-4- yl)-2-((1-acetyl-3- oxoindolin-2- ylidene)methyl) quinolin-6- yl)methyl) (tetrahydro- 2H-pyran-4- yl)carbamate | 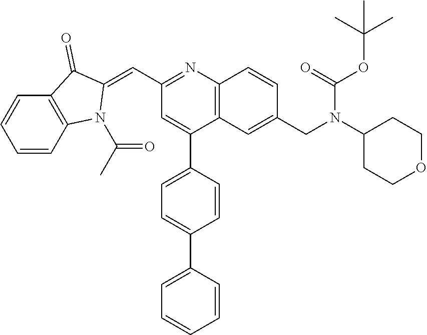 [SMILES: CC(=O)N1/C(=C\c2cc(-c3ccc(-c4ccccc4)cc3)c3cc(CN(C(=O)OC(C)(C)C)C4CCOCC4)ccc3n2)C(=O)c2ccccc21] | Production Ex. 194 | Production Ex. 232 CAS No. 16800- 68- 3 (Reagent Supplier 3) | 1H NMR (400 MHz) δ 8.22 (d, J = 8.4 Hz, 1H), 8.13 (d, J = 8.7 Hz, 1H), 7.85 (dd, J = 7.2, 1.4 Hz, 1H), 7.81-7.76 (m, 3H), 7.72-7.62 (m, 4H), 7.60- 7.55 (m, 3H), 7.55-7.48 (m, 2H), 7.45-7.39 (m, 1H), 7.37 (s, 1H), 7.25 (t, J = 7.5 Hz, 1H), 4.65-4.20 (m, 3H), 3.92 (dd, J = 11.3, 4.4 Hz, 2H), 3.48-3.25 (m, 2H), 2.22 (s, 3H), 1.79- 1.61 (m, 2H), 1.54 (s, 11H). MS 680.29 (M + H). |
| 380 | tert-butyl (Z)-((4- ([1,1′-biphenyl]-3- yl)-2-((1-acetyl-3- oxoindolin-2- ylidene)methyl)qui nolin-6- yl)methyl)(tetrahyd ro-2H-pyran-4- yl)carbamate | 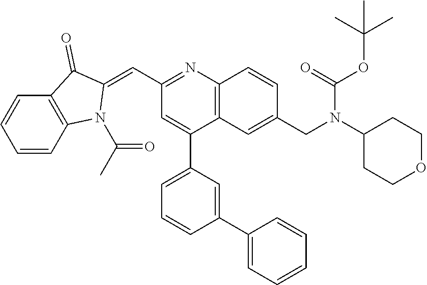 [SMILES: CC(=O)N1/C(=C\c2cc(-c3cccc(-c4ccccc4)c3)c3cc(CN(C(=O)OC(C)(C)C)C4CCOCC4)ccc3n2)C(=O)c2ccccc21] | Production Ex. 194 | Production Ex. 233 CAS No. 16800- 68- 3 (Reagent Supplier 3) | 1H NMR (400 MHz) δ 8.22 (d, J = 8.2 Hz, 1H), 8.13 (d, J = 8.7 Hz, 1H), 7.84 (dd, J = 7.5, 1.4 Hz, 1H), 7.77-7.60 (m, 8H), 7.59 (s, 1H), 7.51-7.44 (m, 3H), 7.42-7.36 (m, 2H), 7.25 (t, J = 7.5 Hz, 1H), 4.65-4.16 (m, 3H), 3.82 (dd, J = 11.5, 4.5 Hz, 2H), 3.39-3.16 (m, 2H), 2.22 (s, 3H), 1.72-1.07 (m, 13H). MS 680.29 (M + H). |
| 381 | tert-butyl (Z)-((2- ((1-acetyl-3- oxoindolin-2- ylidene)methyl)-4- (naphthalen-2- yl)quinolin-6- yl)methyl) (tetrahydro- 2H-pyran-4- yl)carbamate | 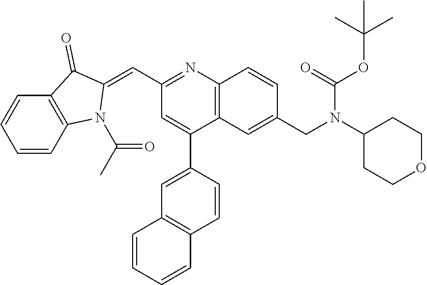 [SMILES: CC(=O)N1/C(=C\c2cc(-c3ccc4ccccc4c3)c3cc(CN(C(=O)OC(C)(C)C)C4CCOCC4)ccc3n2)C(=O)c2ccccc21] | Production Ex. 194 | Production Ex. 234 CAS No. 16800- 68-3 (Reagent Supplier 3) | 1H NMR (400 MHz) δ 8.22 (d, J = 8.3 Hz, 1H), 8.14 (d, J = 8.7 Hz, 1H), 8.04-7.89 (m, 4H), 7.84 (dd, J = 7.5, 1.3 Hz, 1H), 7.73 (s, 1H), 7.69-7.56 (m, 6H), 7.39 (s, 1H), 7.25 (t, J = 7.3 Hz, 1H), 4.64- 4.16 (m, 3H), 3.90 (dd, J = 11.3, 4.5 Hz, 2H), 3.42- 3.25 (m, 2H), 2.23 (s, 3H), 1.66 (s, 13H). MS 654.25 (M + H). |
| 382 | tert-butyl (Z)-((2- ((1-acetyl-3- oxoindolin-2- ylidene)methyl)- [4,8′-biquinolin]-6- yl)methyl)(tetrahydro- 2H-pyran-4- yl)carbamate | 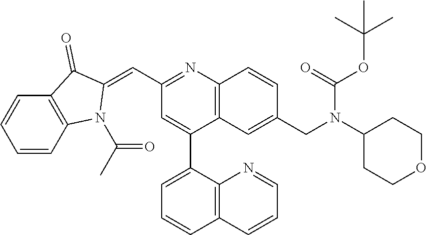 [SMILES: CC(=O)N1/C(=C\c2cc(-c3cccc4cccnc34)c3cc(CN(C(=O)OC(C)(C)C)C4CCOCC4)ccc3n2)C(=O)c2ccccc21] | Production Ex. 194 | Production Ex. 235 CAS No. 16800- 68- 3 (Reagent Supplier 3) | 1H NMR (400 MHz) δ 8.79 (dd, J = 4.1, 1.8 Hz, 1H), 8.30 (dd, J = 8.3, 1.8 Hz, 1H), 8.22 (d, J = 8.3 Hz, 1H), 8.14 (d, J = 8.7 Hz, 1H), 8.04-7.98 (m, 1H), 7.83 (dd, J = 7.6, 1.1 Hz, 1H), 7.73-7.55 (m, 5H), 7.46 (dd, J = 8.3, 4.2 Hz, 1H), 7.40 (s, 1H), 7.24 (t, J = 7.7 Hz, 1H), 7.17 (d, J = 1.9 Hz, 1H), 4.53-3.98 (m, 3H), 3.90-3.78 (m, 2H), 3.40-3.13 (m, 2H), 2.31 (s, 3H), 1.67-0.95 (m, 13H). MS 655.26 (M + H). |
| 383 | 1-acetyl-2-((3- (naphthalen-1-yl)- 6-(N-((2- (trimethylsilyl) ethoxy)methyl) tetrazol- 5-yl)quinolin-2- yl)methylene) indolin-3-one | 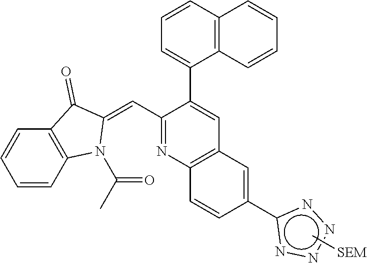 | Production Ex. 194 | Production Ex. 325 CAS No. 16800- 68- 3 (Reagent Supplier 3) | MS 482.3 (M + H) |
| 384 | tert-butyl (Z)-((4- (2-acetyl-1,2,3,4- tetrahydroisoquinolin- 7-yl)-2-((1- acetyl-3- oxoindolin-2- ylidene )methyl) quinolin-6- yl)methyl) (tetrahydro-2H- pyran-4- yl)carbamate | 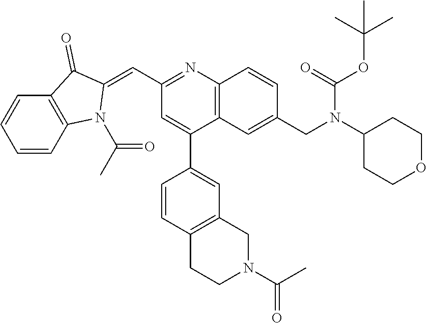 [SMILES: CC(=O)N1CCc2ccc(-c3cc(/C=C4/C(=O)c5ccccc5N4C(C)=O)nc4ccc(CN(C(=O)OC(C)(C)C)C5CCOCC5)cc34)cc2C1] | Production Ex. 194 | Production Ex. 336 CAS No. 16800- 68-3 (Reagent Supplier 3) | 1H NMR (500 MHz) δ 8.21 (d, J = 8.3 Hz, 1H), 8.12 (dd, J = 8.6, 2.8 Hz, 1H), 7.84 (d, J = 7.6 Hz, 1H), 7.71-7.62 (m, 3H), 7.49 (d, J = 8.4 Hz, 1H), 7.38-7.20 (m, 5H), 4.84, 4.73 (each s, combined 2H), 4.63-4.18 (m, 3H), 3.97-3.89 (m, 3H), 3.79 (t, J = 5.9 Hz, 1H), 3.47-3.27 (m, 2H), 3.04, 2.97 (each t, each J = 6.0 Hz, combined 2H), 2.24, 2.23 (each s, combined 3H), 2.21 (s, 3H), 1.80-1.16 (m, 13H). MS 701.5 (M + H). |
| 385 | tert-butyl (Z)-1-(4- ([1,1′-biphenyl]-2- yl)-2-((1-acetyl-3- oxoindolin-2- ylidene)methyl) quinoline-6- carbonyl)piperidine- 4-carboxylate | 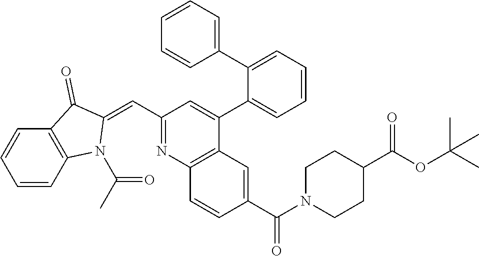 [SMILES: CC(=O)N1/C(=C\c2cc(-c3ccccc3-c3ccccc3)c3cc(C(=O)N4CCC(C(=O)OC(C)(C)C)CC4)ccc3n2)C(=O)c2ccccc21] | Production Ex. 199 | Production Ex.288 C AS No. 16800-68- 3 (Reagent Supplier 3) | 1H NMR (500 MHz) δ 8.23-8.15 (m, 1H), 8.07 (d, J = 8.7 Hz, 1H), 7.85- 7.78 (m, 1H), 7.71-7.48 (m, 6H), 7.41 (dd, J = 7.4, 1.2 Hz, 1H), 7.36 (s, 1H), 7.26-7.21 (m, 1H), 7.18 (s, 1H), 7.06-6.98 (m, 5H), 4.68-4.23 (m, 1H), 3.68-3.40 (m, 1H), 3.17- 2.90 (m, 2H), 2.53-2.44 (m, 1H), 2.08-1.51 (m, 7H), 1.47 (s, 9H). MS 678.52 (M + H). |
| 386 | tert-butyl (Z)-((4- ([1,1′-biphenyl]-2- yl)-2-((1-acetyl-3- oxoindolin-2- ylidene)methyl) quinolin-6- yl)methyl) (tetrahydro-2H- pyran-4- yl)carbamate | 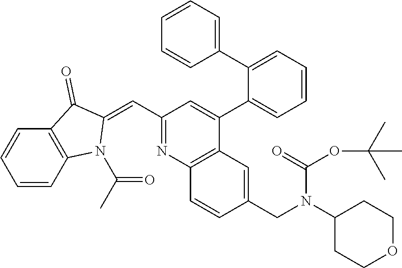 [SMILES: CC(=O)N1/C(=C\c2cc(-c3ccccc3-c3ccccc3)c3cc(CN(C(=O)OC(C)(C)C)C4CCOCC4)ccc3n2)C(=O)c2ccccc21] | Production Ex. 199 | Production Ex. 289 CAS No. 16800- 68- 3 (Reagent Supplier 3) | 1H NMR (500 MHz) δ 8.21-8.16 (m, 1H), 8.00 (d, J = 8.7 Hz, 1H), 7.82- 7.77 (m, 1H), 7.65-7.46 (m, 5H), 7.44 (s, 1H), 7.39- 7.34 (m, 1H), 7.28 (s, 1H), 7.24-7.18 (m, 1H), 7.17 (s, 1H), 7.09-6.95 (m, 5H), 4.57-4.14 (m, 3H), 3.95-3.81 (m, 2H), 3.45-3.20 (m, 2H), 2.00 (s, 3H), 1.26 (s, 13H). MS 680.53 (M + H). |
| 387 | tert-butyl (Z)-4-(2- ((1-acetyl-3- oxoindolin-2- ylidene)methyl)-6- (morpholine-4- carbonyl)quinolin- 4-yl)benzoate | 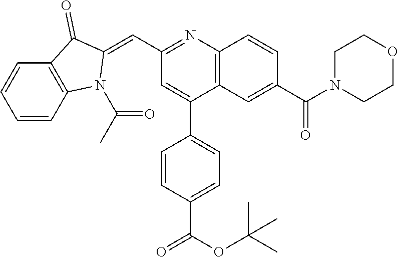 [SMILES: CC(=O)N1/C(=C\c2cc(-c3ccc(C(=O)OC(C)(C)C)cc3)c3cc(C(=O)N4CCOCC4)ccc3n2)C(=O)c2ccccc21] | Production Ex. 199 | Production Ex. 295 CAS No. 16800- 68-3 (Reagent Supplier 3) | 1H NMR (500 MHz) δ 8.23-8.16 (m, 4H), 7.89 (dd, J = 1.9, 0.6 Hz, 1H), 7.86-7.83 (m, 1H), 7.76 (dd, J = 8.6, 1.8 Hz, 1H), 7.70-7.65 (m, 1H), 7.59- 7.54 (m, 3H), 7.35 (s, 1H), 7.29-7.23 (m, 1H), 3.93- 3.33 (m, 8H), 2.21 (s, 3H), 1.66 (s, 9H). MS 604.4 (M + H). |
| 388 | tert-butyl (Z)-3-(2- ((1-acetyl-3- oxoindolin-2- ylidene)methyl)-6- (morpholine-4- carbonyl)quinolin- 4-yl)benzoate | 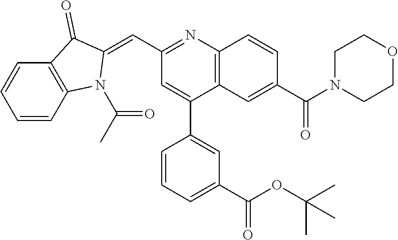 [SMILES: CC(=O)N1/C(=C\c2cc(-c3cccc(C(=O)OC(C)(C)C)c3)c3cc(C(=O)N4CCOCC4)ccc3n2)C(=O)c2ccccc21] | Production Ex. 199 | Production Ex. 298 CAS No. 16800- 68-3 (Reagent Supplier 3) | 1H NMR (500 MHz) δ 8.23-8.15 (m, 3H), 8.11- 8.09 (m, 1H), 7.88-7.83 (m, 2H), 7.77 (dd, J = 8.7, 1.8 Hz, 1H), 7.69-7.61 (m, 3H), 7.58 (s, 1H), 7.35 (s, 1H), 7.28-7.23 (m, 1H), 3.93-3.34 (m, 8H), 2.21 (s, 3H), 1.63 (s, 9H). MS 604.37 (M + H). |
| 389 | tert-butyl (Z)-2-(2- ((1-acetyl-3- oxoindolin-2- ylidene)methyl)-6- (morpholine-4- carbonyl)quinolin- 4-yl)benzoate | 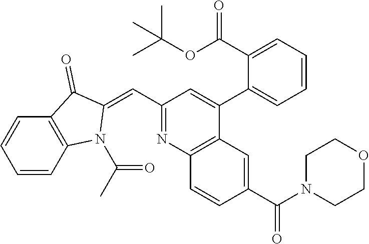 [SMILES: CC(=O)N1/C(=C\c2cc(-c3ccccc3C(=O)OC(C)(C)C)c3cc(C(=O)N4CCOCC4)ccc3n2)C(=O)c2ccccc21] | Production Ex. 199 | Production Ex. 296 CAS No. 16800- 68- 3 (Reagent Supplier 3) | 1H NMR (500 MHz) δ 8.22-8.17 (m, 2H), 8.07 (dd, J = 7.7, 1.6 Hz, 1H), 7.87-7.82 (m, 1H), 7.75 (dd, J = 8.6, 1.9 Hz, 1H), 7.70-7.58 (m, 3H), 7.52 (s, 1H), 7.49 (d, J = 1.8 Hz, 1H), 7.36-7.30 (m, 2H), 7.29-7.22 (m, 1H), 3.97- 3.22 (m, 8H), 2.21 (s, 3H), 0.88 (s, 9H). MS 604.39 (M + H). |
| 390 | (Z)-1-acetyl-2-((6- (morpholine-4- carbonyl)-4-(3-(1- trityl-1H-pyrazol-4- yl)phenyl)quinolin-2- yl)methylene) indolin-3-one | 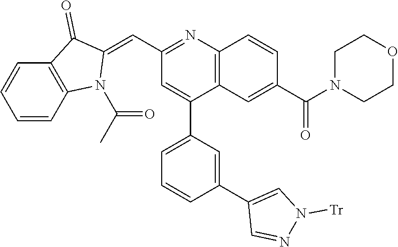 | Production Ex. 199 | Production Ex. 299 CAS No. 16800- 68-3 (Reagent Supplier 3) | 1H NMR (500 MHz) δ 8.21-8.16 (m, 2H), 7.99 (s, 1H), 7.94-7.91 (m, 1H), 7.84 (dd, J = 7.6, 1.4 Hz, 1H), 7.77-7.71 (m, 2H), 7.68-7.63 (m, 1H), 7.61-7.57 (m, 2H), 7.53- 7.48 (m, 2H), 7.38-7.29 (m, 11H), 7.27-7.18 (m, 7H), 3.91-3.28 (m, 8H), 2.19 (s, 3H). MS 812.45 (M + H). |
| 391 | (Z)-1-acetyl-2-((6- (morpholine-4- carbonyl)-4-(1- trityl-1H-pyrazol- 4-yl)quinolin-2- yl)methylene) indolin-3-one | 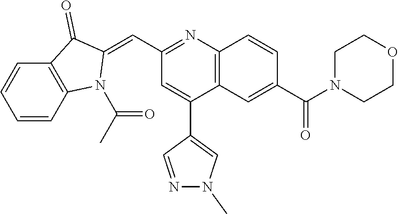 | Production Ex. 199 | Production Ex. 300 CAS No. 16800- 68- (Reagent Supplier 3) | 1H NMR (500 MHz) δ 8.20-8.13 (m, 3H), 8.02 (d, J = 0.8 Hz, 1H), 7.85- 7.82 (m, 1H), 7.76 (d, J = 0.8 Hz, 1H), 7.73 (dd, J = 8.5, 1.9 Hz, 1H), 7.68- 7.63 (m, 1H), 7.55 (s, 1H), 7.40-7.35 (m, 9H), 7.29 (s, 1H), 7.27-7.22 (m, 7H), 3.95-3.31 (m, 8H), 2.12 (s, 3H). MS 736.36 (M + H). |
| 392 | tert-butyl (Z)-((2- ((1-acetyl-3- oxoindolin-2- ylidene)methyl)-4- (1-methyl-1H- pyrazol-4- yl)quinolin-6- yl)methyl)(tetrahydro- 2H-pyran-4- yl)carbamate | 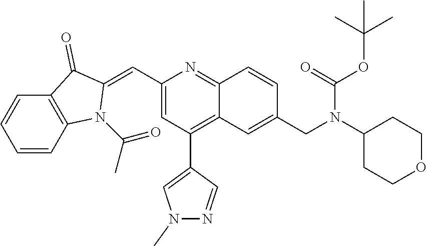 [SMILES: CC(=O)N1/C(=C\c2cc(-c3cnn(C)c3)c3cc(CN(C(=O)OC(C)(C)C)C4CCOCC4)ccc3n2)C(=O)c2ccccc21] | Production Ex. 199 | Production Ex. 305 CAS No. 16800- 68-3 (Reagent Supplier 3) | 1H NMR (500 MHz) δ 8.24-8.14 (m, 1H), 8.11- 7.88 (m, 2H), 7.85-7.56 (m, 5H), 7.54-7.47 (m, 1H), 7.34-7.16 (m, 2H), 4.69-4.22 (m, 3H), 4.05 (s, 3H), 3.97-3.84 (m, 2H), 3.50-3.23 (m, 2H), 2.15 (s, 3H), 1.79-1.12 (m, 13H). MS 608.41 (M + H). |
| 393 | 1-acetyl-2-((6- (morpholine-4- carbonyl)-4-(N- ((2- (trimethylsilyl) ethoxy)methyl)-1,2,4- triazol-3- yl)quinolin-2- yl)methylene) indolin-3-one | 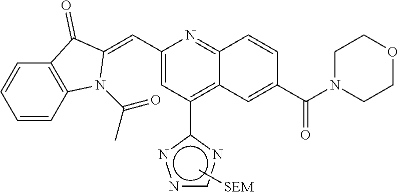 | Production Ex. 199 | Production Ex. 313 CAS No. 16800- 68- 3 (Reagent Supplier 3) | MS 625.36 (M + H). |
| 394 | (Z)-1-acetyl-2-((4- (1-((2- (trimethylsilyl) ethoxy)methyl)-1H- pyrazol-4- yl)quinolin-2- yl)methylene ) indolin-3-one | 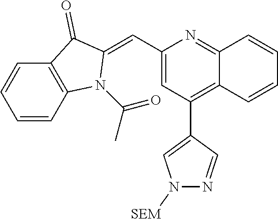 [SMILES: CC(=O)N1/C(=C\c2cc(-c3cnn(COCC[Si](C)(C)C)c3)c3ccccc3n2)C(=O)c2ccccc21] | Production Ex. 199 | Production Ex. 314 CAS No. 16800- 68-3 (Reagent Supplier 3) | 1H NMR (500 MHz) δ 8.21 (dd, J = 8.3, 0.8 Hz, 1H), 8.17-8.12 (m, 2H), 7.98 (s, 1H), 7.91 (s, 1H), 7.85-7.81 (m, 1H), 7.78- 7.73 (m, 1H), 7.67-7.63 (m, 1H), 7.60-7.55 (m, 2H), 7.33 (s, 1H), 7.24 (t, J = 7.4 Hz, 1H), 5.58 (s, 2H), 3.74-3.68 (m, 2H), 2.16 (s, 3H), 1.01-0.96 (m, 2H), 0.02 (s, 9H). MS 511.2 (M + H). |
| 395 | tert-butyl (Z)-((2- ((1-acetyl-3- oxoindolin-2- ylidene)methyl)-4- (2-(1-methyl-1H- pyrazol-4- yl)phenyl) quinolin-6- yl)methyl) (tetrahydro-2H- pyran-4- yl)carbamate | 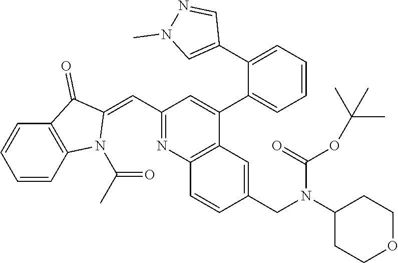 [SMILES: CC(=O)N1/C(=C\c2cc(-c3ccccc3-c3cnn(C)c3)c3cc(CN(C(=O)OC(C)(C)C)C4CCOCC4)ccc3n2)C(=O)c2ccccc21] | Production Ex. 199 | Production Ex. 326 CAS No. 16800- 68-3 (Reagent Supplier 3) | 1H NMR (500 MHz) δ 8.18 (d, J = 8.3 Hz, 1H), 8.09 (d, J = 8.6 Hz, 1H), 7.83 (dd, J = 7.5, 1.2 Hz, 1H), 7.68-7.63 (m, 1H), 7.62-7.56 (m, 2H), 7.54- 7.49 (m, 1H), 7.46-7.21 (m, 6H), 7.09 (s, 1H), 6.74 (s, 1H), 4.58-4.14 (m, 3H), 3.94-3.80 (m, 2H), 3.59 (s, 3H), 3.44-3.22 (m, 2H), 2.18 (s, 3H), 1.69- 1.11 (m, 13H). MS 684.4 (M + H). |
| 396 | tert-butyl ((4-(2- acetyl-1,2,3,4- tetrahydroisoquinolin- 5-yl)-2-((1- acetyl-3- oxoindolin-2- ylidene)methyl) quinolin-6- yl)methyl)(tetrahydro- 2H-pyran-4- yl)carbamate | 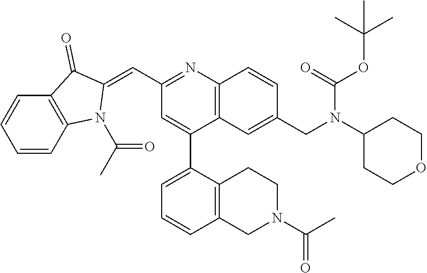 [SMILES: CC(=O)N1CCc2c(cccc2-c2cc(/C=C3/C(=O)c4ccccc4N3C(C)=O)nc3ccc(CN(C(=O)OC(C)(C)C)C4CCOCC4)cc23)C1] | Production Ex. 199 | Production Ex. 332 CAS No. 16800- 68- 3 (Reagent Supplier 3) | MS 701.4 (M + H). |
| 397 | tert-butyl (Z)-((2- ((1-acetyl-3- oxoindolin-2- ylidene)methyl) quinolin-4- yl)methyl) (tetrahydro-2H- pyran-4- yl)carbamate | 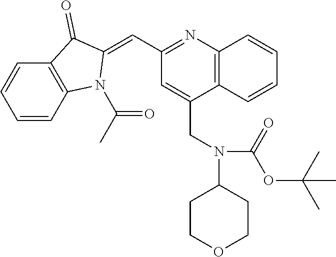 [SMILES: CC(=O)N1/C(=C\c2cc(CN(C(=O)OC(C)(C)C)C3CCOCC3)c3ccccc3n2)C(=O)c2ccccc21] | Production Ex. 199 | Production Ex. 333 CAS No. 16800- 68-3 (Reagent Supplier 3) | 1H NMR (500 MHz) δ 8.20 (d, J = 8.3 Hz, 1H), 8.15 (d, J = 8.5 Hz, 1H), 7.94 (d, J = 8.3 Hz, 1H), 7.83 (d, J = 7.5 Hz, 1H), 7.76 (t, J = 7.5 Hz, 1H), 7.67-7.58 (m, 2H), 7.43 (s, 1H), 7.28 (s, 1H), 7.24 (t, J = 7.5 Hz, 1H), 5.03- 4.75 (m, 2H), 4.65-4.05 (m, 1H), 4.00-3.94 (m, 2H), 3.57-3.33 (m, 2H), 2.14 (s, 3H), 1.86-1.06 (m, 13H). MS 528.4 (M + H). |
| 398 | tert-butyl (Z)-((4- (2-acetyl-1,2,3,4- tetrahydroisoquinolin- 6-yl)-2-((1- acetyl-3- oxoindolin-2- ylidene)methyl)qui nolin-6- yl)methyl)(tetrahyd ro-2H-pyran-4- yl)carbamate | 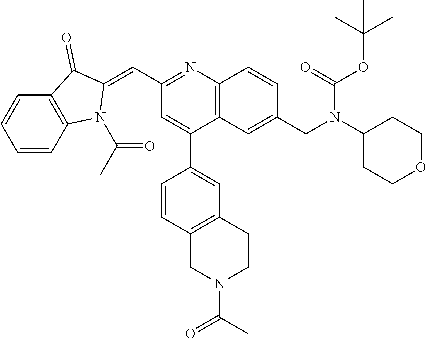 [SMILES: CC(=O)N1CCc2cc(-c3cc(/C=C4/C(=O)c5ccccc5N4C(C)=O)nc4ccc(CN(C(=O)OC(C)(C)C)C5CCOCC5)cc34)ccc2C1] | Production Ex. 199 | Production Ex. 334 CAS No. 16800- 68- 3 (Reagent Supplier 3) | 1H NMR (500 MHz) δ 8.21 (d, J = 8.3 Hz, 1H), 8.12 (dd, J = 8.8, 2.0 Hz, 1H), 7.84 (dd, J = 7.5, 1.3 Hz, 1H), 7.71-7.61 (m, 3H), 7.50 (s, 1H), 7.36- 7.23 (m, 5H), 4.86, 4.75 (each s, combined 2H), 4.63-4.21 (m, 3H), 3.96- 3.89 (m, 3H), 3.79 (t, J = 5.9 Hz, 1H), 3.46-3.28 (m, 2H), 3.02, 2.95 (each t, each J = 5.9 Hz, combined 2H), 2.26, 2.24 (each s, combined 3H), 2.21 (s, 3H), 1.77-1.12 (m, 13H). MS 701.4 (M + H). |
| 399 | tert-butyl (Z)-((2- ((1-acetyl-3- oxoindolin-2- ylidene)methyl)-4- (3-(1-methyl-1H- pyrazol-4- yl)phenyl)quinolin-6- yl)methyl)(tetrahydro- 2H-pyran-4- yl)carbamate | 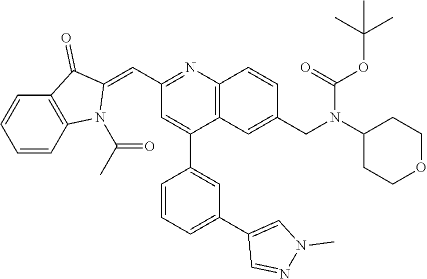 [SMILES: CC(=O)N1/C(=C\c2cc(-c3cccc(-c4cnn(C)c4)c3)c3cc(CN(C(=O)OC(C)(C)C)C4CCOCC4)ccc3n2)C(=O)c2ccccc21] | Production Ex. 199 | Production Ex. 335 CAS No. 16800- 68- 3 (Reagent Supplier 3) | 1H NMR (500 MHz) δ 8.22 (d, J = 8.3 Hz, 1H), 8.13 (d, J = 8.7 Hz, 1H), 7.84 (dd, J = 7.6, 1.4 Hz, 1H), 7.81 (s, 1H), 7.74- 7.70 (m, 2H), 7.69-7.61 (m, 3H), 7.60-7.47 (m, 3H), 7.37 (s, 1H), 7.33 (d, J = 7.6 Hz, 1H), 7.27-7.22 (m, 1H), 4.66-4.18 (m, 3H), 3.97 (s, 3H), 3.88 (dd, J = 11.4, 4.5 Hz, 2H), 3.45- 3.22 (m, 2H), 2.21 (s, 3H), 1.77-1.05 (m, 13H). MS 684.5 (M + H). |
| 400 | 4-([1,1′-biphenyl]- 2-yl)-2-methyl-N- (tetrahydro-2H- pyran-4- yl)quinoline-6- carboxamide | 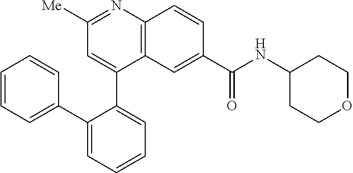 [SMILES: Cc1cc(-c2ccccc2-c2ccccc2)c2cc(C(=O)NC3CCOCC3)ccc2n1] | Production Ex. 34 | Production Ex. 94 CAS No. 38041- 19- 9 (Reagent Supplier 13) | 1H NMR (500 MHz) δ 8.00-7.96 (m, 1H), 7.88- 7.83 (m, 2H), 7.63-7.57 (m, 2H), 7.55-7.50 (m, 1H), 7.45-7.41 (m, 1H), 7.22 (s, 1H), 7.10-7.02 (m, 5H), 5.79 (d, J = 7.9 Hz, 1H), 4.22-4.11 (m, 1H), 4.06-3.99 (m, 2H), 3.58-3.49 (m, 2H), 2.71 (s, 3H), 2.04-1.98 (m, |
| | | | | | 2H), 1.64-1.52 (m, |
| | | | | | 2H). MS 423.31 (M + H). |
| 401 | (2-methyl-3- (naphthalen-1- yl)quinolin-6- yl)(morpholino) methanone | 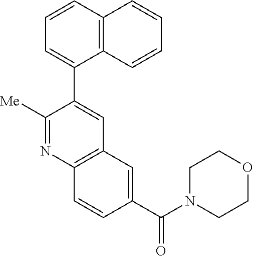 [SMILES: Cc1nc2ccc(C(=O)N3CCOCC3)cc2cc1-c1cccc2ccccc12] | Production Ex. 34 | Production Ex. 422 | 1H NMR (500 MHz) δ 8.19-8.16 (m, 1H), 8.07 (s, 1H), 7.99-7.95 (m, 2H), 7.91 (d, J = 1.9 Hz, 1H), 7.76 (dd, J = 8.6, 1.9 Hz, 1H), 7.60 (dd, J = 8.3, 7.0 Hz, 1H), 7.56-7.51 (m, 1H), 7.44-7.36 (m, 3H), 4.03-3.42 (m, 8H), 2.46 (s, 3H). MS 383.29 (M + H). |
| 402 | (2-methyl-4- (pyrimidin-5- yl)quinolin-6- yl)(morpholino) methanone | 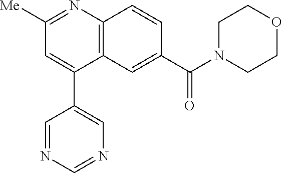 [SMILES: Cc1cc(-c2cncnc2)c2cc(C(=O)N3CCOCC3)ccc2n1] | Production Ex. 34 | Production Ex. 423 | 1H NMR (500 MHz) δ 9.38 (s, 1H), 8.91 (s, 2H), 8.18 (d, J = 8.6 Hz, 1H), 7.80 (d, J = 1.9 Hz, 1H), 7.75 (dd, J = 8.6, 1.8 Hz, 1H), 7.32 (s, 1H), 3.94- 3.34 (m, 8H), 2.84 (s, 3H). MS 335.22 (M + H). |
| 403 | 4-chloro-2-methyl- N-(tetrahydro-2H- pyran-4- yl)quinoline-6- carboxamide | 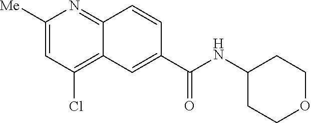 [SMILES: Cc1cc(Cl)c2cc(C(=O)NC3CCOCC3)ccc2n1] | Production Ex. 34 | Production Ex. 92 CAS No. 38041- 19- 9 (Reagent Supplier 13) | 1H NMR (500 MHz) δ 8.58-8.55 (m, 1H), 8.12- 8.05 (m, 2H), 7.47 (s, 1H), 6.17 (d, J = 7.9 Hz, 1H), 4.33-4.23 (m, 1H), 4.08- 4.01 (m, 2H), 3.61-3.53 (m, 2H), 2.75 (s, 3H), 2.11- 2.04 (m, 2H), 1.70-1.60 |
| | | | | | (m, 2H). MS 305.17 (M + H). |
| 404 | (2-methylquinolin- 4-yl)(morpholino) methanone | 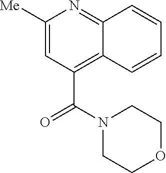 [SMILES: Cc1cc(C(=O)N2CCOCC2)c2ccccc2n1] | Production Ex. 34 | CAS No. 634-38- 8 (Reagent Supplier 3) | 1H NMR (500 MHz) δ 8.06 (d, J = 8.4 Hz, 1H), 7.79-7.75 (m, 1H), 7.75- 7.71 (m, 1H), 7.57-7.52 (m, 1H), 7.22 (s, 1H), 4.05- 3.97 (m, 1H), 3.91-3.81 (m, 3H), 3.60-3.49 (m, 2H), 3.25-3.14 (m, 2H), 2.77 (s, 3H). MS 257.22 (M + H). |
| 405 | (2-methyl-4-(1- methyl-1H-1,2,4- triazol-3- yl)quinolin-6- yl)(morpholino) methanone | 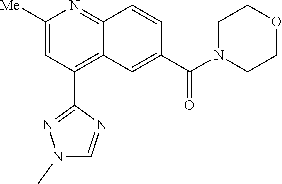 [SMILES: Cc1cc(-c2ncn(C)n2)c2cc(C(=O)N3CCOCC3)ccc2n1] | Production Ex. 34 | Production Ex. 424 | 1H NMR (500 MHz) δ 9.29 (d, J = 1.7 Hz, 1H), 8.21 (s, 1H), 8.11 (dd, J = 8.6, 0.6 Hz, 1H), 8.07 (s, 1H), 7.76 (dd, J = 8.6, 1.9 Hz, 1H), 4.09 (s, 3H), 3.96- 3.50 (m, 8H), 2.82 (s, 3H). MS 338.24 (M + H). |
| 406 | (2-methyl-4-(1- methyl-1H- imidazol-2- yl)quinolin-6- yl)(morpholino) methanone | 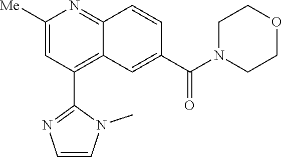 [SMILES: Cc1cc(-c2nccn2C)c2cc(C(=O)N3CCOCC3)ccc2n1] | Production Ex. 34 | Production Ex. 425 | 1H NMR (500 MHz) δ 8.11 (d, J = 8.6 Hz, 1H), 8.02 (d, J = 2.0 Hz, 1H), 7.73 (dd, J = 8.6, 1.9 Hz, 1H), 7.39 (s, 1H), 7.27 (d, J = 1.3 Hz, 1H), 7.13 (d, J = 1.3 Hz, 1H), 3.90-3.39 (m, 11H), 2.81 (s, 3H). MS 337.26 (M + H). |
| 407 | (2-methyl-4- (pyrimidin-2- yl)quinolin-6- yl)(morpholino) methanone | 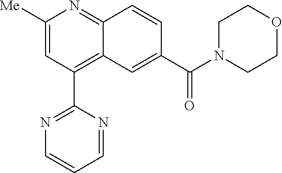 [SMILES: Cc1cc(-c2ncccn2)c2cc(C(=O)N3CCOCC3)ccc2n1] | Production Ex. 34 | Production Ex. 426 | 1H NMR (500 MHz) δ 8.97 (d, J = 4.9 Hz, 2H), 8.86 (d, J = 1.8 Hz, 1H), 8.13 (d, J = 8.6 Hz, 1H), 7.96 (s, 1H), 7.73 (dd, J = 8.6, 1.9 Hz, 1H), 7.41 (t, J = 4.9 Hz, 1H), 3.93-3.44 (m, 8H), 2.83 (s, 3H). MS 335.23 (M + H). |
| 408 | (2-methylquinolin- 4-yl)(4-(oxetan-3- yl)piperazin-1- yl)methanone | 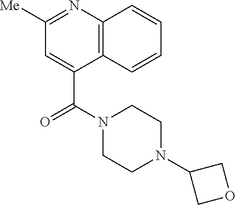 [SMILES: Cc1cc(C(=O)N2CCN(C3COC3)CC2)c2ccccc2n1] | Production Ex. 34 | CAS No. 634-38- 8 (Reagent Supplier 3) CAS No. 1254115- 23- 5 (Reagent Supplier 15) | 1H NMR (500 MHz) δ 8.06 (d, J = 8.3 Hz, 1H), 7.77-7.69 (m, 2H), 7.55- 7.50 (m, 1H), 7.21 (s, 1H), 4.68-4.61 (m, 2H), 4.60- 4.54 (m, 2H), 4.08-4.01 (m, 1H), 3.93-3.85 (m, 1H), 3.55-3.48 (m, 1H), 3.26-3.17 (m, 2H), 2.75 (s, 3H), 2.53-2.42 (m, 2H), 2.23-2.10 (m, 2H). MS 312.26 (M + H). |
| 409 | (2-methyl-4-(N- ((2- (trimethylsilyl)ethoxy) methyl)-1,2,4- triazol-3- yl)quinolin-6- yl)(morpholino) methanone | 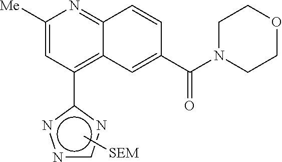 | Production Ex. 34 | Production Ex. 427 | 1H NMR (500 MHz) δ 9.29, 8.21 (each d, each J = 1.9 Hz, combined 1H), 8.41, 7.71 (each s, combined 1H), 8.16-8.09 (m, 2H), 7.80-7.75 (m, 1H), 5.64, 5.41 (each s, combined 2H), 3.97-3.45 (m, 10H), 2.84, 2.82 (each s, combined 3H), 1.26- |
| | | | | | 0.92 (m, 2H), 0.02, 0.01 |
| | | | | | (each s, combined |
| | | | | | 9H). MS 454.30 (M + H). |
| 410 | (2-methyl-4- (pyridazin-3- yl)quinolin-6- yl)(morpholino) methanone | 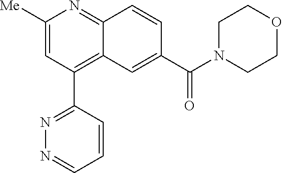 [SMILES: Cc1cc(-c2cccnn2)c2cc(C(=O)N3CCOCC3)ccc2n1] | Production Ex. 34 | Production Ex. 428 | 1H NMR (500 MHz) δ 9.36 (dd, J = 5.0, 1.7 Hz, 1H), 8.16 (d, J = 8.6 Hz, 1H), 8.09 (d, J = 1.8 Hz, 1H), 7.80 (dd, J = 8.4, 1.7 Hz, 1H), 7.75 (dd, J = 8.7, 1.8 Hz, 1H), 7.71 (dd, J = 8.4, 5.0 Hz, 1H), 7.52 (s, 1H), 3.91-3.38 (m, 8H), 2.85 (s, 3H). MS 335.1 (M + H). |
| 411 | (2-methyl-4-(1- methyl-1H- imidazol-4- yl)quinolin-6- yl)(morpholino)me thanone | 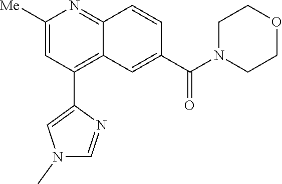 [SMILES: Cc1cc(-c2cn(C)cn2)c2cc(C(=O)N3CCOCC3)ccc2n1] | Production Ex. 34 | Production Ex. 429 | 1H NMR (500 MHz) 8 8.72 (d, J = 1.9 Hz, 1H), 8.03 (d, J = 8.6 Hz, 1H), 7.66 (dd, J = 8.6, 1.9 Hz, 1H), 7.59 (s, 1H), 7.57 (s, 1H), 7.35 (d, J = 1.3 Hz, 1H), 3.91-3.41 (m, 11H), 2.73 (s, 3H). MS 337.1 (M + H). |
| 412 | (2-methyl-4- (pyrazin-2- yl)quinolin-6- yl)(morpholino)me thanone | 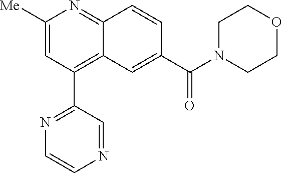 [SMILES: Cc1cc(-c2cnccn2)c2cc(C(=O)N3CCOCC3)ccc2n1] | Production Ex. 34 | Production Ex. 430 | 1H NMR (500 MHz) δ 8.95-8.92 (m, 1H), 8.81- 8.78 (m, 1H), 8.74 (d, J = 2.5 Hz, 1H), 8.21 (d, J = 1.8 Hz, 1H), 8.15 (d, J = 8.6 Hz, 1H), 7.77-7.73 (m, 1H), 7.50 (s, 1H), 3.91- 3.41 (m, 8H), 2.85 (s, 3H). MS 335.1 (M + H). |
| 413 | (4-(1H-indazol-4- yl)-2- methylquinolin-6- yl)(morpholino) methanone | 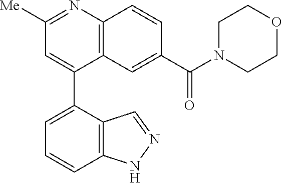 [SMILES: Cc1cc(-c2cccc3[nH]ncc23)c2cc(C(=O)N3CCOCC3)ccc2n1] | Production Ex. 45 | Production Ex. 214 | 1H NMR (500 MHz) δ 10.96 (s, 1H), 8.19 (dd, J = 8.6, 0.6 Hz, 1H), 7.79 (d, J = 1.8 Hz, 1H), 7.75 (dd, J = 8.6, 1.9 Hz, 1H), 7.73 (d, J = 1.1 Hz, 1H), 7.64-7.60 (m, 1H), 7.54-7.50 (m, 1H), 7.46 (s, 1H), 7.24 (dd, J = 7.0, 0.8 Hz, 1H), 3.89- 3.24 (m, 8H), 2.85 (s, 3H). MS 373.25 (M + H). |
| 414 | tert-butyl ((4- ([1,1′-biphenyl]-2- yl)-2- methylquinolin-6- yl)methyl)(tetrahydro- 2H-pyran-4- yl)carbamate | 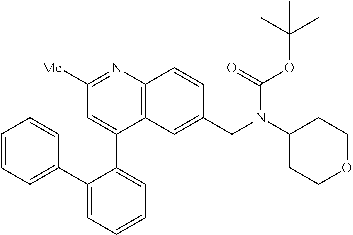 [SMILES: Cc1cc(-c2ccccc2-c2ccccc2)c2cc(CN(C(=O)OC(C)(C)C)C3CCOCC3)ccc2n1] | Production Ex. 112 | Production Ex. 212 | 1H NMR (500 MHz) δ 7.92 (d, J = 8.7 Hz, 1H), 7.57-7.51 (m, 2H), 7.50- 7.43 (m, 2H), 7.41 (s, 1H), 7.34 (d, J = 7.5 Hz, 1H), 7.09-7.01 (m, 6H), 4.60- 4.12 (m, 3H), 3.94-3.82 (m, 2H), 3.44-3.20 (m, 2H), 2.62 (s, 3H), 1.72- 1.11 (m, 13H). MS 509.39 (M + H). |
| 415 | tert-butyl ((2- methyl-4-(1- methyl-1H- pyrazol-4- yl)quinolin-6- yl)methyl)(tetrahydro- 2H-pyran-4- yl)carbamate | 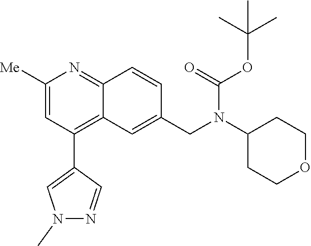 [SMILES: Cc1cc(-c2cnn(C)c2)c2cc(CN(C(=O)OC(C)(C)C)C3CCOCC3)ccc2n1] | Production Ex. 112 | Production Ex. 213 | 1H NMR (500 MHz) δ 8.00 (d, J = 8.6 Hz, 1H), 7.94 (s, 1H), 7.78 (s, 1H), 7.69 (s, 1H), 7.58 (dd, J = 8.6, 1.9 Hz, 1H), 7.23 (s, 1H), 4.70-4.19 (m, 3H), 4.04 (s, 3H), 3.98-3.85 (m, 2H), 3.49-3.21 (m, 2H), 2.73 (s, 3H), 1.63 (s, 13H). MS 437.44 (M + H). |
| 416 | tert-butyl ((4- ([1,1′-biphenyl]-4- yl)-2- methylquinolin-6- yl)methyl)(tetrahydro- 2H-pyran-4- yl)carbamate | 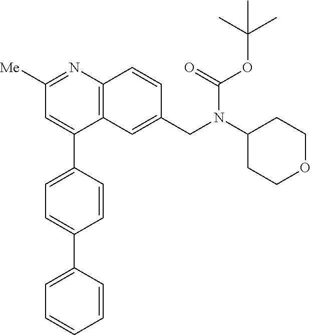 [SMILES: Cc1cc(-c2ccc(-c3ccccc3)cc2)c2cc(CN(C(=O)OC(C)(C)C)C3CCOCC3)ccc2n1] | Production Ex. 112 | Production Ex. 248 | 1H NMR (400 MHz) δ 8.05 (d, J = 8.7 Hz, 1H), 7.77-7.73 (m, 3H), 7.71- 7.66 (m, 2H), 7.61-7.47 (m, 5H), 7.44-7.38 (m, 1H), 7.27 (s, 1H), 4.67- 4.15 (m, 3H), 3.91 (dd, J = 11.3, 4.3 Hz, 2H), 3.50- 3.22 (m, 2H), 2.78 (s, 3H), 1.80-1.04 (m, 13H). MS 509.45 (M + H). |
| 417 | tert-butyl ((4- ([1,1′-biphenyl]-3- yl)-2- methylquinolin-6- yl)methyl)(tetrahydro- 2H-pyran-4- yl)carbamate | 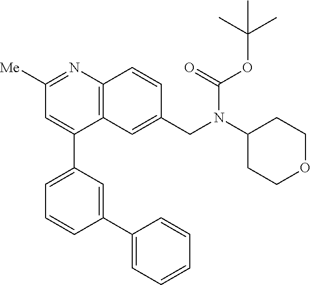 [SMILES: Cc1cc(-c2cccc(-c3ccccc3)c2)c2cc(CN(C(=O)OC(C)(C)C)C3CCOCC3)ccc2n1] | Production Ex. 112 | Production Ex. 249 | 1H NMR (400 MHz) δ 8.04 (d, J = 8.7 Hz, 1H), 7.74-7.54 (m, 7H), 7.50- 7.35 (m, 4H), 7.28 (s, 1H), 4.65-4.09 (m, 3H), 3.82 (dd, J = 11.4, 4.5 Hz, 2H), 3.41-3.12 (m, 2H), 2.78 (s, 3H), 1.80-1.02 (m, 13H). MS 509.51 (M + H). |
| 418 | tert-butyl ((2- methyl-4- (naphthalen-2- yl)quinolin-6- yl)methyl)(tetrahydro- 2H-pyran-4- yl)carbamate | 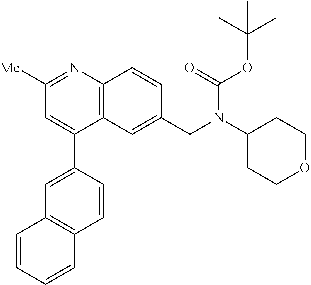 [SMILES: Cc1cc(-c2ccc3ccccc3c2)c2cc(CN(C(=O)OC(C)(C)C)C3CCOCC3)ccc2n1] | Production Ex. 112 | Production Ex. 250 | 1H NMR (400 MHz) δ 8.06 (d, J = 8.6 Hz, 1H), 7.99 (d, J = 8.3 Hz, 1H), 7.97-7.87 (m, 3H), 7.69 (d, J = 1.8 Hz, 1H), 7.62- 7.54 (m, 4H), 7.32 (s, 1H), 4.65-4.09 (m, 3H), 3.89 (dd, J = 11.5, 4.5 Hz, 2H), 3.43-3.20 (m, 2H), 2.80 (s, 3H), 1.75-1.07 (m, 13H). MS 483.45 (M + H) |
| 419 | tert-butyl ((2- methyl-[4,8′- biquinolin]-6- yl)methyl)(tetrahydro- 2H-pyran-4- yl)carbamate | 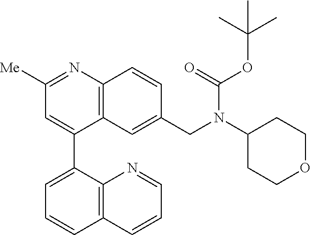 [SMILES: Cc1cc(-c2cccc3cccnc23)c2cc(CN(C(=O)OC(C)(C)C)C3CCOCC3)ccc2n1] | Production Ex. 112 | Production Ex. 251 | 1H NMR (400 MHz) δ 8.78 (dd, J = 4.3, 1.8 Hz, 1H), 8.28 (dd, J = 8.3, 1.8 Hz, 1H), 8.04 (d, J = 8.6 Hz, 1H), 8.01-7.95 (m, 1H), 7.70-7.64 (m, 2H), 7.52 (d, J = 8.8 Hz, 1H), 7.44 (dd, J = 8.3, 4.1 Hz, 1H), 7.33 (s, 1H), 7.08 (d, J = 1.9 Hz, 1H), 4.48-3.94 (m, 3H), 3.88-3.76 (m, 2H), 3.39-3.09 (m, 2H), 2.80 (s, 3H), 1.74-0.94 (m, 13H). MS 484.44 (M + H). |
| 420 | tert-butyl ((4- chloro-2- methylquinolin-6- yl)methyl)(tetrahydro- 2H-pyran-4- yl)carbamate | 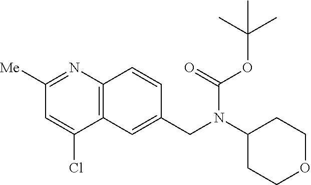 [SMILES: Cc1cc(Cl)c2cc(CN(C(=O)OC(C)(C)C)C3CCOCC3)ccc2n1] | Production Ex. 112 | Production Ex. 441 | 1H NMR (500 MHz) δ 8.03-7.92 (m, 2H), 7.61 (d, J = 8.8 Hz, 1H), 7.39 (s, 1H), 4.76-4.32 (m, 3H), 4.00-3.90 (m, 2H), 3.51- 3.27 (m, 2H), 2.71 (s, 3H), 1.81-1.31 (m, 13H). MS 391.4 (M + H). |
| 421 | tert-butyl ((2- methylquinolin-4- yl)methyl)(tetrahydro- 2H-pyran-4- yl)carbamate | 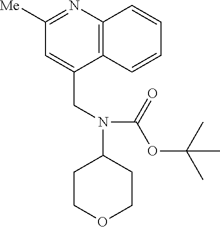 [SMILES: Cc1cc(CN(C(=O)OC(C)(C)C)C2CCOCC2)c2ccccc2n1] | Production Ex. 112 | Production Ex. 268 | 1H NMR (500 MHz) δ 8.06 (d, J = 8.5 Hz, 1H), 7.90 (d, J = 8.5 Hz, 1H), 7.70 (t, J = 7.6 Hz, 1H), 7.53 (t, J = 7.6 Hz, 1H), 7.12 (s, 1H), 4.96-4.73 (m, 2H), 4.61-4.43 (m, 1H), 3.99-3.92 (m, 2H), 3.55-3.28 (m, 2H), 2.72 (s, 3H), 1.84-1.11 (m, 13H). MS 357.3 (M + H). |
| 422 | 2-methyl-3- (naphthalen-1- yl)quinoline-6- carboxylic acid | 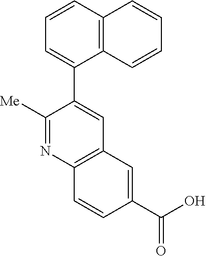 [SMILES: Cc1nc2ccc(C(=O)O)cc2cc1-c1cccc2ccccc12] | Production Ex. 90 | Production Ex. 340 or Production Ex. 341 | 1H NMR (500 MHz, dimethylsulfoxide-d6) δ 8.69 (d, J = 2.0 Hz, 1H), 8.44 (s, 1H), 8.24 (dd, J = 8.8, 1.9 Hz, 1H), 8.13- 8.10 (m, 1H), 8.09-8.05 (m, 2H), 7.67 (dd, J = 8.3, 7.0 Hz, 1H), 7.60-7.56 (m, 1H), 7.54 (dd, J = 6.9, 1.2 Hz, 1H), 7.50-7.45 (m, 1H), 7.36-7.32 (m, 1H), 2.34 (s, 3H). MS 314.28 (M + H). |
| 423 | 2-methyl-4- (pyrimidin-5- yl)quinoline-6- carboxylic acid | 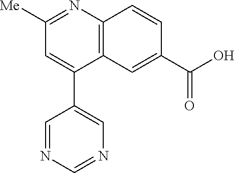 [SMILES: Cc1cc(-c2cncnc2)c2cc(C(=O)O)ccc2n1] | Production Ex. 90 | Production Ex. 240 | MS 266.11 (M + H). |
| 424 | 2-methyl-4-(1- methyl-1H-1,2,4- triazol-3- yl)quinoline-6- carboxylic acid | 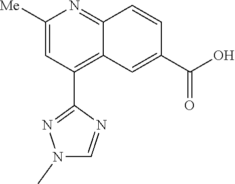 [SMILES: Cc1cc(-c2ncn(C)n2)c2cc(C(=O)O)ccc2n1] | Production Ex. 90 | Production Ex. 241 | MS 269.15 (M + H). |
| 425 | 2-methyl-4-(1- methyl-1H- imidazol-2- yl)quinoline-6- carboxylic acid | 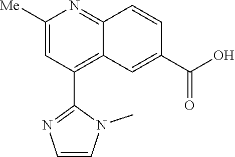 [SMILES: Cc1cc(-c2nccn2C)c2cc(C(=O)O)ccc2n1] | Production Ex. 90 | Production Ex. 242 | MS 268.16 (M + H). |
| 426 | 2-methyl-4- (pyrimidin-2- yl)quinoline-6- carboxylic acid | 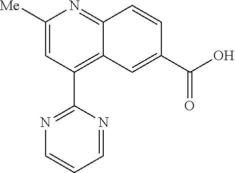 [SMILES: Cc1cc(-c2ncccn2)c2cc(C(=O)O)ccc2n1] | Production Ex. 90 | Production Ex. 243 | MS 266.12 (M + H). |
| 427 | 2-methyl-4-(N-((2- (trimethylsilyl)etho xy)methyl)-1,2,4- triazol-3- yl)quinoline-6- carboxylic acid | 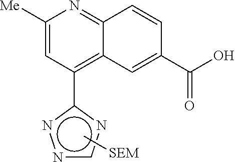 | Production Ex. 90 | Production Ex. 244 | MS 385.27 (M + H). |
| 428 | 2-methyl-4- (pyridazin-3- yl)quinoline-6- carboxylic acid | 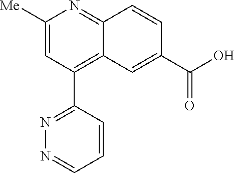 [SMILES: Cc1cc(-c2cccnn2)c2cc(C(=O)O)ccc2n1] | Production Ex. 90 | Production Ex. 245 | MS 266.12 (M + H). |
| 429 | 2-methyl-4-(1- methyl-1H- imidazol-4- yl)quinoline-6- carboxylic acid | 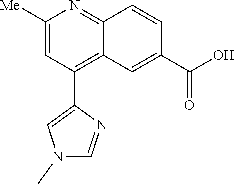 [SMILES: Cc1cc(-c2cn(C)cn2)c2cc(C(=O)O)ccc2n1] | Production Ex. 90 | Production Ex. 246 | MS 268.14 (M + H). |
| 430 | 2-methyl-4- (pyrazin-2- yl)quinoline-6- carboxylic acid | 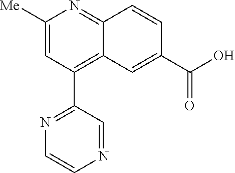 [SMILES: Cc1cc(-c2cnccn2)c2cc(C(=O)O)ccc2n1] | Production Ex. 90 | Production Ex. 247 | 1H NMR (500 MHz dimethylsulfoxide-d6) 8 9.13-9.10 (m, 1H), 8.98- 8.93 (m, 1H), 8.89-8.85 (m, 1H), 8.78-8.74 (m, 1H), 8.26-8.20 (m, 1H), 8.11 (dd, J = 8.8, 2.0 Hz, 1H), 7.79 (d, J = 2.0 Hz, 1H), 2.78 (s, 3H). MS 266.1 (M + H). |
| 431 | 2-methyl-3-(1- methyl-1H- pyrazol-4- yl)quinoline-6- carboxylic acid | 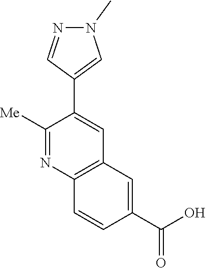 [SMILES: Cc1nc2ccc(C(=O)O)cc2cc1-c1cnn(C)c1] | Production Ex. 90 | Production Ex. 364 | MS 268.1 (M + H). |
| 432 | tert-butyl 4-([1,1′- biphenyl]-3-yl)-2- methylquinoline-6- carboxylate | 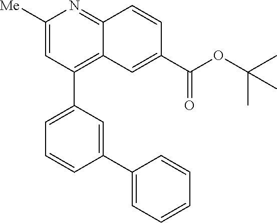 [SMILES: Cc1cc(-c2cccc(-c3ccccc3)c2)c2cc(C(=O)OC(C)(C)C)ccc2n1] | Production Ex. 85 | Production Ex. 215 | 1H NMR (400 MHz) δ 8.68 (d, J = 1.8 Hz, 1H), 8.24 (dd, J = 9.0, 1.9 Hz, 1H), 8.10 (d, J = 8.7 Hz, 1H), 7.76-7.71 (m, 2H), 7.68-7.54 (m, 3H), 7.53- 7.35 (m, 5H), 2.81 (s, 3H), 1.56 (s, 9H). MS 396.20 (M + H). |
| 433 | tert-butyl 2- methyl-4- (naphthalen-1- yl)quinoline-6- carboxylate | 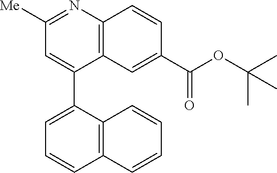 [SMILES: Cc1cc(-c2cccc3ccccc23)c2cc(C(=O)OC(C)(C)C)ccc2n1] | Production Ex. 85 | Production Ex. 216 | 1H NMR (400 MHz) δ 8.21 (dd, J = 8.7, 1.9 Hz, 1H), 8.15-8.10 (m, 2H), 7.99 (d, J = 7.9 Hz, 1H), 7.96 (d, J = 8.2 Hz, 1H), 7.60 (dd, J = 8.4, 7.1 Hz, 1H), 7.54-7.48 (m, 1H), 7.46 (dd, J = 7.0, 1.3 Hz, 1H), 7.39-7.32 (m, 3H), 2.83 (s, 3H), 1.47 (s, 9H). MS 370.18 (M + H). |
| 434 | tert-butyl 4-([1,1′- biphenyl]-4-yl)-2- methylquinoline-6- carboxylate | 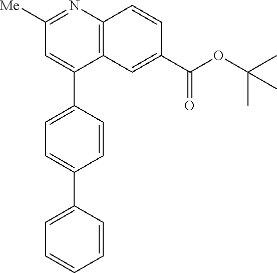 [SMILES: Cc1cc(-c2ccc(-c3ccccc3)cc2)c2cc(C(=O)OC(C)(C)C)ccc2n1] | Production Ex. 85 | Production Ex. 217 | 1H NMR (400 MHz) δ 8.69 (d, J = 1.9 Hz, 1H), 8.24 (dd, J = 8.9, 2.1 Hz, 1H), 8.10 (d, J = 8.8 Hz, 1H), 7.80-7.74 (m, 2H), 7.71-7.66 (m, 2H), 7.63- 7.58 (m, 2H), 7.53-7.47 (m, 2H), 7.44-7.38 (m, 1H), 7.35 (s, 1H), 2.82 (s, 3H), 1.59 (s, 9H). MS 396.22 (M + H). |
| 435 | tert-butyl 2- methyl-[4,8′- biquinoline]-6- carboxylate | 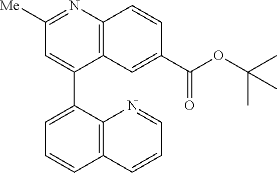 [SMILES: Cc1cc(-c2cccc3cccnc23)c2cc(C(=O)OC(C)(C)C)ccc2n1] | Production Ex. 85 | Production Ex. 218 | 1H NMR (400 MHz) δ 8.81 (dd, J = 4.2, 1.8 Hz, 1H), 8.28 (dd, J = 8.3, 1.8 Hz, 1H), 8.18 (dd, J = 8.8, 1.8 Hz, 1H), 8.12-8.07 (m, 2H), 7.99 (dd, J = 7.3, 2.3 Hz, 1H), 7.74-7.65 (m, 2H), 7.45 (dd, J = 8.3, 4.3 Hz, 1H), 7.42 (s, 1H), 2.83 (s, 3H), 1.48 (s, 9H). MS 371.19 (M + H). |
| 436 | tert-butyl 2- methyl-4- (naphthalen-2- yl)quinoline-6- carboxylate | 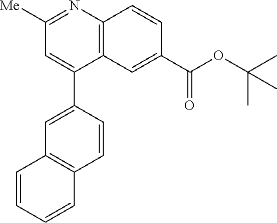 [SMILES: Cc1cc(-c2ccc3ccccc3c2)c2cc(C(=O)OC(C)(C)C)ccc2n1] | Production Ex. 85 | Production Ex. 219 | 1H NMR (400 MHz) δ 8.65 (d, J = 1.9 Hz, 1H), 8.24 (dd, J = 8.8, 2.0 Hz, 1H), 8.11 (d, J = 8.9 Hz, 1H), 8.03-7.99 (m, 2H), 7.98-7.90 (m, 2H), 7.63 (dd, J = 8.3, 1.8 Hz, 1H), 7.61-7.54 (m, 2H), 7.40 (s, 1H), 2.83 (s, 3H), 1.55 (s, 9H). MS 370.19 (M + H). |
| 437 | 6-(2- (hydroxymethyl)- 6-(morpholine-4- carbonyl)quinolin- 4-yl)-3,4- dihydroisoquinolin- 1(2H)-one | 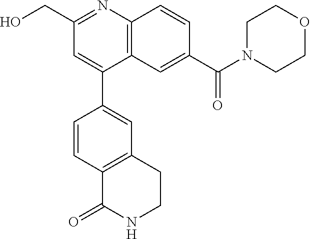 [SMILES: O=C1NCCc2cc(-c3cc(CO)nc4ccc(C(=O)N5CCOCC5)cc34)ccc21] | Production Ex. 111 | Production Ex. 344 | 1H NMR (500 MHz) δ 8.24 (d, J = 7.9 Hz, 1H), 8.20 (dd, J = 8.6, 0.6 Hz, 1H), 7.91 (dd, J = 1.8, 0.6 Hz, 1H), 7.76 (dd, J = 8.6, 1.9 Hz, 1H), 7.46 (dd, J = 7.9, 1.7 Hz, 1H), 7.36- 7.34 (m, 1H), 7.30 (s, 1H), 6.04 (s, 1H), 4.98 (d, J = 4.7 Hz, 2H), 4.27 (t, J = 4.8 Hz, 1H), 3.92-3.31 (m, 10H), 3.11 (t, J = 6.6 Hz, 2H). MS 418.25 (M + H). |
| 438 | 6-(morpholine-4- carbonyl)-4-(1- oxo-1,2,3,4- tetrahydroisoquinol in-6-yl)quinoline- 2-carbaldehyde | 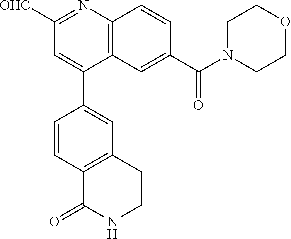 [SMILES: O=Cc1cc(-c2ccc3c(c2)CCNC3=O)c2cc(C(=O)N3CCOCC3)ccc2n1] | Production Ex. 188 | Production Ex. 437 | 1H NMR (500 MHz) δ 10.27 (s, 1H), 8.39 (d, J = 8.7 Hz, 1H), 8.26 (d, J = 7.9 Hz, 1H), 8.02 (s, 1H), 8.00 (d, J = 1.8 Hz, 1H), 7.85 (dd, J = 8.6, 1.8 Hz, 1H), 7.49 (dd, J = 7.9, 1.8 Hz, 1H), 7.39 (d, J = 1.7 Hz, 1H), 6.62 (d, J = 11.1 Hz, 1H), 3.91-3.34 (m, 10H), 3.12 (t, J = 6.6 Hz, 2H). MS 416.31 (M + H). |
| 439 | 4-chloro-2- methylquinoline-6- carbaldehyde | 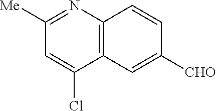 [SMILES: Cc1cc(Cl)c2cc(C=O)ccc2n1] | Production Ex. 188 | Production Ex. 440 | 1H NMR (500 MHz) 10.22 (s, 1H), 8.70 (d, J = 1.8 Hz, 1H), 8.22 (dd, J = 8.7, 1.8 Hz, 1H), 8.13 (d, J = 8.7 Hz, 1H), 7.51 (s, 1H), 2.77 (s, 3H). MS 206.0 (M + H). |
| 440 | (4-chloro-2- methylquinolin-6- yl)methanol | 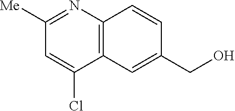 [SMILES: Cc1cc(Cl)c2cc(CO)ccc2n1] | | Production Ex. 66 | 1H NMR (500 MHz) δ 8.17-8.14 (m, 1H), 8.02 (d, J = 8.6 Hz, 1H), 7.74 (dd, J = 8.7, 1.9 Hz, 1H), 7.41 (s, 1H), 4.92 (d, J = 5.9 Hz, 2H), 2.72 (s, 3H), 1.97 (t, J = 6.0 Hz, |
| | | | | | 1H). MS 208.0 (M + H). |
| 441 | N-((4-chloro-2- methylquinolin-6- yl)methyl)tetrahydro- 2H-pyran-4- amine | 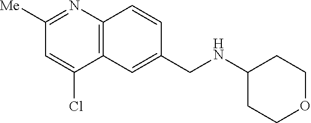 [SMILES: Cc1cc(Cl)c2cc(CNC3CCOCC3)ccc2n1] | Production Ex. 72 | Production Ex. 439 | 1H NMR (500 MHz) δ 8.10-8.07 (m, 1H), 7.99 (d, J = 8.6 Hz, 1H), 7.74 (dd, J = 8.6, 1.9 Hz, 1H), 7.39 (s, 1H), 4.04 (s, 2H), 4.02-3.97 (m, 2H), 3.45- 3.36 (m, 2H), 2.82-2.73 |
| | | | | | (m, 1H), 2.72 (s, 3H), 1.95- |
| | | | | | 1.86 (m, 2H), 1.55-1.44 |
| | | | | | (m, 2H). MS 291.1 (M + H). |

### Example 1

A mixture of 2-(4-formyl-2-methoxyphenoxy)acetamide (European Journal of Medicinal Chemistry, 81, 1-14, 2014; 1.80 g), 1-acetylindolin-3-one (manufactured by Combi-Blocks; 1.51 g), toluene (50 ml), Molecular Sieve 4A (10 g), and piperidine (0.17 ml) was stirred at 110° C. for 17 hours, and then, at room temperature for 2 hours.

Thereafter, the reaction mixture was filtrated, and the obtained solid was washed with toluene and was then suspended in chloroform (300 ml), followed by stirring the suspension at 50° C. for 1 hour.

An insoluble matter in the mixture was removed by filtration, and the solvent was distilled away from the filtrate under reduced pressure. Thereafter, ethyl acetate was added to the obtained residue, and the solvent was then distilled away again.

Ethyl acetate and hexane were added to the obtained residue, followed by pulverization. The obtained solid was washed with ethyl acetate to obtain (Z)-2-(4-((1-acetyl-3-oxoindolin-2-ylidene)methyl)-2-methoxyphenoxy)acetamide (2.526 g) in the form of a yellow solid.

### Example 2

A mixture of 3-methoxy-4-(2-morpholino-2-oxoethoxy)benzaldehyde (manufactured by Enamine; 95 mg), 1-acetylindolin-3-one (manufactured by Combi-Blocks; 60 mg), toluene (3 ml), Molecular Sieve 4A (1 g), and piperidine (one droplet) was stirred at under heat reflux for 16 hours. Thereafter, the reaction mixture was purified by silica gel column chromatography (eluent: chloroform-methanol), so as to obtain (Z)-1-acetyl-2-(3-methoxy-4-(2-morpholino-2-oxoethoxy)benzylidene)indolin-3-one (50 mg) in the form of a yellow oily substance.

### Example 47

A mixture of 5-(morpholine-4-carbonyl)benzo[d]thiazole-2-carbaldehyde (Production Example 131; 55 mg), 1-acetylindolin-3-one (manufactured by Combi-Blocks; 35 mg), toluene (3 ml), Molecular Sieve 4A (1 g), and piperidine (0.0039 ml) was stirred at 80° C. for 3 hours.

Thereafter, the reaction mixture was purified by silica gel column chromatography (eluent: chloroform-methanol), so as to obtain the previously eluted low-polarity (Z)-1-acetyl-2-((5-(morpholine-4-carbonyl)benzo[d]thiazol-2-yl)methylene)indolin-3-one (25 mg) in the form of a brown oily substance.

### Example 48

In the silica gel column chromatography, in which the compound of Example 47 was obtained, the subsequently eluted high-polarity (E)-1-acetyl-2-((5-(morpholine-4-carbonyl)benzo[d]thiazol-2-yl)methylene)indolin-3-one (15 mg) was obtained in the form of a yellow solid.

### Example 49

Using 2-(4-formyl-2-methoxyphenoxy)acetamide (European Journal of Medicinal Chemistry, 81, 1-14; 2014), 1-benzoylindolin-3-one (Heterocycles, 92(6), 1063-1074, 2016), toluene, Molecular Sieve 4A, and piperidine, 2-(4-((1-benzoyl-3-oxoindolin-2-ylidene)methyl)-2-methoxyphenoxy)acetamide was obtained in the form of a yellow oily substance in the same manner as that of Example 2.

### Example 50

Using 3-methoxy-4-(2-morpholino-2-oxoethoxy)benzaldehyde (manufactured by Enamine), 1-acetyl-5-fluoroindolin-3-one (manufactured by Aurora Fine Chemicals), toluene, Molecular Sieve 4A, and piperidine, 1-acetyl-5-fluoro-2-(3-methoxy-4-(2-morpholino-2-oxoethoxy)benzylidene)indolin-3-one was obtained in the form of a yellow oily substance in the same manner as that of Example 2.

### Example 51

Using 3-methoxy-4-(2-morpholino-2-oxoethoxy)benzaldehyde (manufactured by Enamine), 1-acetyl-1,2-dihydro-3H-pyrrolo[2,3-b]pyridin-3-one (manufactured by Aurora Fine Chemicals), toluene, Molecular Sieve 4A, and piperidine, 1-acetyl-2-(3-methoxy-4-(2-morpholino-2-oxoethoxy)benzylidene)-1,2-dihydro-3H-pyrrolo[2,3-b]pyridin-3-one was obtained in the form of a light brown oily substance in the same manner as that of Example 2.

### Example 52

Using (E)-2-(4-formyl-2-methoxyphenyl)ethene-1-sulfonamide (Production Example 5), 1-acetylindolin-3-one (manufactured by Combi-Blocks), toluene, DMF, Molecular Sieve 4A, and piperidine, (1E)-2-(4-((1-acetyl-3-oxoindolin-2-ylidene)methyl)-2-methoxyphenyl)ethene-1-sulfonamide was obtained in the form of a yellow oily substance in the same manner as that of Example 2.

### Example 53

A mixture of N-((2-formylbenzo[d]thiazol-5-yl)methyl)methanesulfonamide (Production Example 141; 61 mg), 1-acetylindolin-3-one (manufactured by Combi-Blocks; 40 mg), toluene (3 ml), Molecular Sieve 4A (1 g), and piperidine (0.0045 ml) was stirred at 80° C. for 4 hours.

Thereafter, the reaction mixture was purified by silica gel column chromatography (eluent: chloroform-methanol), and was then crystallized from chloroform, so as to obtain (E)-N-((2-((1-acetyl-3-oxoindolin-2-ylidene)methyl)benzo[d]thiazol-5-yl)methyl)methanesulfonamide (40.9 mg) in the form of a yellow solid.

### Example 56

Using N-((2-formylbenzo[d]thiazol-6-yl)methyl)methanesulfonamide (Production Example 145), 1-acetylindolin-3-one (manufactured by Combi-Blocks), toluene, THF, Molecular Sieve 4A, and piperidine, (E)-N-((2-((1-acetyl-3-oxoindolin-2-ylidene)methyl)benzo[d]thiazol-6-yl)methyl)methanesulfonamide was obtained in the form of a yellow solid in the same manner as that of Example 53.

### Example 57

A mixture of 2-(4-formyl-2-methoxyphenoxy)acetic acid (manufactured by Enamine; 68 mg), 1-acetylindolin-3-one (manufactured by Combi-Blocks; 57 mg), toluene (3 ml), Molecular Sieve 4A (1 g), and piperidine (one droplet) was stirred at under heat reflux for 24 hours.

Thereafter, the reaction mixture was successively purified by silica gel column chromatography (eluent: chloroform-methanol-acetic acid) and then, with a preparative silica gel thin-layer plate (developing solvent: chloroform-methanol), so as to obtain 2-(4-((1-acetyl-3-oxoindolin-2-ylidene)methyl)-2-methoxyphenoxy)acetic acid (32 mg) in the form of a yellow solid.

### Example 58

A mixture of 6-(morpholine-4-carbonyl)benzo[d]thiazole-2-carbaldehyde (Production Example 126; 572 mg), 1-acetylindolin-3-one (manufactured by Combi-Blocks; 363 mg), toluene (20 ml), Molecular Sieve 4A (1 g), and piperidine (0.0409 ml) was stirred at 80° C. for 40 minutes, and the reaction mixture was then filtrated. The obtained filtrate was used in Example 59.

Chloroform and methanol were added to the obtained solid, and Molecular Sieve 4A was then removed by filtration. After that, the solvent was distilled away from the filtrate under reduced pressure. The obtained residue was purified by silica gel column chromatography (eluent: chloroform-methanol), so as to obtain (E)-1-acetyl-2-((6-(morpholine-4-carbonyl)benzo[d]thiazol-2-yl)methylene)indolin-3-one (506 mg) in the form of a yellow solid.

### Example 59

The filtrate obtained by filtrating the reaction mixture of Example 58 was concentrated under reduced pressure, and the obtained residue was then purified by silica gel column chromatography (eluent: chloroform-methanol), so as to obtain (Z)-1-acetyl-2-((6-(morpholine-4-carbonyl)benzo[d]thiazol-2-yl)methylene)indolin-3-one (27 mg) in the form of a brown oily substance.

### Example 60

A mixture of 6-(1,1-dioxidothiomorpholine-4-carbonyl)quinoline-2-carbaldehyde (Production Example 132; 42 mg), 1-acetylindolin-3-one (manufactured by Combi-Blocks; 21 mg), toluene (3 ml), Molecular Sieve 4A (1 g), and piperidine (0.0118 ml) was stirred at 80° C. for 1 hour 45 minutes. Thereafter, the reaction mixture was successively purified by silica gel column chromatography (eluent: chloroform-methanol) and then, by gel permeation chromatography (eluent: chloroform), so as to obtain (Z)-1-acetyl-2-((6-(1,1-dioxidothiomorpholine-4-carbonyl)quinolin-2-yl)methylene)indolin-3-one (28 mg) in the form of a brown oily substance.

### Example 98

A mixture of 5-(2,6-dimethylmorpholine-4-carbonyl)benzo[d]thiazole-2-carbaldehyde (Production Example 142; 245 mg), 1-acetylindolin-3-one (manufactured by Combi-Blocks; 141 mg), toluene (5 ml), Molecular Sieve 4A (1.5 g), and piperidine (0.0159 ml) was stirred at 80° C. for 1 hour 30 minutes, and the reaction mixture was then purified by silica gel column chromatography (eluent: chloroform-methanol). The previously eluted low-polarity fraction was collected, and the solvent was then distilled away under reduced pressure. The obtained residue was purified by gel permeation chromatography (eluent: chloroform), so as to obtain (Z)-1-acetyl-2-((5-(2,6-dimethylmorpholine-4-carbonyl)benzo[d]thiazol-2-yl)methylene)indolin-3-one (37 mg) in the form of a brown oily substance.

### Example 99

In the silica gel column chromatography, in which the compound of Example 98 was obtained, the subsequently eluted high-polarity fraction was collected, and the solvent was then distilled away under reduced pressure. The obtained residue was purified by gel permeation chromatography (eluent: chloroform), so as to obtain (E)-1-acetyl-2-((5-(2,6-dimethylmorpholine-4-carbonyl)benzo[d]thiazol-2-yl)methylene)indolin-3-one (86 mg) in the form of a brown oily substance.

### Example 102

Using 5-(2-morpholino-2-oxoethoxy)picolinaldehyde (manufactured by Aurora Fine Chemicals), 1-acetyl-1,2-dihydro-3H-pyrrolo[2,3-b]pyridin-3-one (manufactured by Aurora Fine Chemicals), toluene, Molecular Sieve 4A, and piperidine, 1-acetyl-2-((5-(2-morpholino-2-oxoethoxy)pyridin-2-yl)methylene)-1,2-dihydro-3H-pyrrolo[2,3-b]pyridin-3-one was obtained in the form of a yellow oily substance in the same manner as that of Example 60.

### Example 103

Using 3-methoxy-4-(2-morpholino-2-oxoethoxy)benzaldehyde (manufactured by Enamine), 1-acetyl-6-fluoroindolin-3-one (manufactured by Aurora Fine Chemicals), toluene, Molecular Sieve 4A, and piperidine, 1-acetyl-6-fluoro-2-(3-methoxy-4-(2-morpholino-2-oxoethoxy)benzylidene)indolin-3-one was obtained in the form of a yellow oily substance in the same manner as that of Example 60.

### Example 104

Using 3-methoxy-4-(2-morpholino-2-oxoethoxy)benzaldehyde (manufactured by Enamine), 1-acetyl-4-fluoroindolin-3-one (manufactured by Aurora Fine Chemicals), toluene, Molecular Sieve 4A, and piperidine, 1-acetyl-4-fluoro-2-(3-methoxy-4-(2-morpholino-2-oxoethoxy)benzylidene)indolin-3-one was obtained in the form of a yellow oily substance in the same manner as that of Example 60.

### Example 105

Using 3-methoxy-4-(2-morpholino-2-oxoethoxy)benzaldehyde (manufactured by Enamine), 1-acetyl-7-fluoroindolin-3-one (manufactured by Aurora Fine Chemicals), toluene, Molecular Sieve 4A, and piperidine, 1-acetyl-7-fluoro-2-(3-methoxy-4-(2-morpholino-2-oxoethoxy)benzylidene)indolin-3-one was obtained in the form of a yellow oily substance in the same manner as that of Example 60.

### Example 106

Using 6-(morpholine-4-carbonyl)quinoline-2-carbaldehyde (Production Example 119), N-((1-acetyl-3-oxoindolin-4-yl)methyl)acetamide (Production Example 2), toluene, Molecular Sieve 4A, and piperidine, (Z)—N-((1-acetyl-2-((6-(morpholine-4-carbonyl)quinolin-2-yl)methylene)-3-oxoindolin-4-yl)methyl)acetamide was obtained in the form of a yellow oily substance in the same manner as that of Example 60.

### Example 107

Using 6-((1,1-dioxidothiomorpholino)methyl)quinoline-2-carbaldehyde (Production Example 133), 1-acetylindolin-3-one (manufactured by Combi-Blocks), toluene, THF, Molecular Sieve 4A, and piperidine, (Z)-1-acetyl-2-((6-((1,1-dioxidothiomorpholino)methyl)quinolin-2-yl)methylene)indolin-3-one was obtained in the form of a brown oily substance in the same manner as that of Example 60.

### Example 108

Using (E)-6-(3-morpholino-3-oxoprop-1-en-1-yl)benzo[d]thiazole-2-carbaldehyde (Production Example 148), 1-acetylindolin-3-one (manufactured by Combi-Blocks), toluene, DMF, Molecular Sieve 4A, and piperidine, (E)-1-acetyl-2-((6-((E)-3-morpholino-3-oxoprop-1-en-1-yl)benzo[d]thiazol-2-yl)methylene)indolin-3-one was obtained in the form of a yellow solid in the same manner as that of Example 60.

### Example 109

A mixture of (E)-5-(3-morpholino-3-oxoprop-1-en-1-yl)benzo[d]thiazole-2-carbaldehyde (Production Example 139; 79 mg), 1-acetylindolin-3-one (manufactured by Combi-Blocks; 47 mg), toluene (6 ml), DMF (1 ml), Molecular Sieve 4A (1 g), and piperidine (0.0258 ml) was stirred at 80° C. for 3 hours 30 minutes, and the reaction mixture was then purified by silica gel column chromatography (eluent: hexane-chloroform-methanol).

The previously eluted low-polarity fraction was collected, and the solvent was then distilled away under reduced pressure. The obtained residue was purified by gel permeation chromatography (eluent: chloroform), so as to obtain (Z)-1-acetyl-2-((5-((E)-3-morpholino-3-oxoprop-1-en-1-yl)benzo[d]thiazol-2-yl)methylene)indolin-3-one (25 mg) in the form of a brown oily substance.

### Example 110

In the silica gel column chromatography, in which the compound of Example 109 was obtained, the subsequently eluted high-polarity fraction was collected, and the solvent was then distilled away under reduced pressure. The obtained residue was purified by gel permeation chromatography (eluent: chloroform), so as to obtain (E)-1-acetyl-2-((5-((E)-3-morpholino-3-oxoprop-1-en-1-yl)benzo[d]thiazol-2-yl)methylene)indolin-3-one (24 mg) in the form of a brown oily substance.

### Example 111

Using 2-formyl-6-(morpholine-4-carbonyl)quinoline-4-carboxamide (Production Example 182), 1-acetylindolin-3-one (manufactured by Combi-Blocks), DMF, Molecular Sieve 4A, and piperidine, (Z)-2-((1-acetyl-3-oxoindolin-2-ylidene)methyl)-6-(morpholine-4-carbonyl)quinoline-4-carboxamide was obtained in the form of a brown solid in the same manner as that of Example 60.

### Example 114

A mixture of 6-(4-phenylpiperidine-1-carbonyl)quinoline-2-carbaldehyde (Production Example 128; 104 mg), 1-acetylindolin-3-one (manufactured by Combi-Blocks; 47 mg), toluene (3 ml), Molecular Sieve 4A (1 g), and piperidine (0.0052 ml) was stirred at 80° C. for 3 hours. Thereafter, the reaction mixture was successively purified by silica gel column chromatography (eluent: hexane-chloroform), then by gel permeation chromatography (eluent: chloroform), and then, with a preparative silica gel thin-layer plate (developing solvent: chloroform-methanol), so as to obtain (Z)-1-acetyl-2-((6-(4-phenylpiperidine-1-carbonyl)quinolin-2-yl)methylene)indolin-3-one (19 mg) in the form of a yellow oily substance.

### Example 115

A mixture of 6-(4-morpholinopiperidine-1-carbonyl)quinoline-2-carbaldehyde (Production Example 129; 95 mg), 1-acetylindolin-3-one (manufactured by Combi-Blocks; 48 mg), toluene (3 ml), Molecular Sieve 4A (1 g), and piperidine (0.0053 ml) was stirred at 80° C. for 3 hours. Thereafter, the reaction mixture was successively purified by silica gel column chromatography (eluent: chloroform-methanol), gel permeation chromatography (eluent: chloroform), and silica gel column chromatography (eluent: chloroform-methanol), so as to obtain (Z)-1-acetyl-2-((6-(4-morpholinopiperidine-1-carbonyl)quinolin-2-yl)methylene)indolin-3-one (62 mg) in the form of a brown oily substance.

### Example 117

A mixture of (2-methyl-[4,4′-biquinolin]-6-yl)(morpholino)methanone (Production Example 102; 19 mg), 1,4-dioxane (0.76 ml), and selenium dioxide (11 mg) was stirred at 80° C. for 2 hours, and an insoluble matter in the reaction mixture was then removed by filtration. After that, the solvent was distilled away from the filtrate under reduced pressure. The obtained residue was purified by silica gel column chromatography (eluent: chloroform-methanol). Using the obtained crude 6-(morpholine-4-carbonyl)-[4,4′-biquinoline]-2-carbaldehyde, 1-acetylindolin-3-one (manufactured by Combi-Blocks), toluene, Molecular Sieve 4A, and piperidine, (E)-1-acetyl-2-((6-(morpholine-4-carbonyl)-[4,4′-biquinolin]-2-yl)methylene)indolin-3-one was obtained in the form of a brown solid in the same manner as that of Example 60.

### Example 119

A mixture of (2-methylquinolin-6-yl)(piperazin-1-yl)methanone (manufactured by Aurora Fine Chemicals; 206 mg), (3-methoxyoxetan-3-yl)methyl 4-methylbenzenesulfonate (Production Example 58; 222 mg), acetonitrile (4 ml), and potassium carbonate (141 mg) was stirred at 80° C. for 24 hours, and the solvent was then distilled away from the reaction mixture under reduced pressure.

The obtained residue was purified by silica gel column chromatography (eluent: chloroform-methanol), and a fraction containing (4-((3-methoxyoxetan-3-yl)methyl)piperazin-1-yl)(2-methylquinolin-6-yl)methanone was then collected. After that, the solvent was distilled away under reduced pressure.

A mixture of the obtained residue, selenium dioxide (74 mg), and 1,4-dioxane (2.4 ml) was stirred at 80° C. for 3 hours 30 minutes, and an insoluble matter in the reaction mixture was then removed by filtration. After that, the solvent was distilled away from the filtrate under reduced pressure. The obtained residue was purified by silica gel column chromatography (eluent: chloroform-methanol), and a fraction containing 6-(4-((3-methoxyoxetan-3-yl)methyl)piperazine-1-carbonyl)quinoline-2-carbaldehyde was then collected. After that, the solvent was distilled away under reduced pressure.

A mixture of the obtained residue, 1-acetylindolin-3-one (manufactured by Combi-Blocks; 37 mg), toluene (3 ml), Molecular Sieve 4A (1 g), and piperidine (0.0041 ml) was stirred at 80° C. for 1 hour 30 minutes, and the reaction mixture was successively purified by silica gel column chromatography (eluent: chloroform-methanol) and then, by gel permeation chromatography (eluent: chloroform), so as to obtain (Z)-1-acetyl-2-((6-(4-((3-methoxyoxetan-3-yl)methyl)piperazine-1-carbonyl)quinolin-2-yl)methylene)indolin-3-one (37 mg) in the form of a brown oily substance.

### Example 120

In the gel permeation chromatography, in which the compound of Example 119 was obtained, the previously eluted high-molecular-weight fraction was collected, and the solvent was then distilled away under reduced pressure, so as to obtain (3-methoxyoxetan-3-yl)methyl (Z)-4-(2-((1-acetyl-3-oxoindolin-2-ylidene)methyl)quinoline-6-carbonyl)piperazine-1-carboxylate (15 mg) in the form of a brown oily substance.

### Example 122

A mixture of 2-(3-methoxy-4-(2-morpholino-2-oxoethoxy)benzylidene)indolin-3-one (Production Example 89; 30 mg), DMF (0.3 ml), 60% sodium hydride (6 mg), and methyl iodide (0.0095 ml) was stirred at room temperature for 1 hour.

Thereafter, the solvent was removed from the reaction mixture under a nitrogen flow, and the obtained residue was then purified by silica gel column chromatography (eluent: chloroform-methanol), so as to obtain 2-(3-methoxy-4-(2-morpholino-2-oxoethoxy)benzylidene)-1-methylindolin-3-one (7 mg) in the form of a red oily substance.

### Example 123

A mixture of 2-(3-methoxy-4-(2-morpholino-2-oxoethoxy)benzylidene)indolin-3-one (Production Example 89; 30.1 mg), DMF (0.3 ml), and 60% sodium hydride (6.1 mg) was stirred at room temperature for 30 minutes, and diethyl dicarbonate (0.0169 ml) was then added to the reaction mixture under ice cold, followed by stirring the obtained mixture at room temperature for 16 hours.

Thereafter, the solvent was removed from the reaction mixture under a nitrogen flow, and the obtained residue was then purified by silica gel column chromatography (eluent: chloroform-methanol), so as to obtain ethyl 2-(3-methoxy-4-(2-morpholino-2-oxoethoxy)benzylidene)-3-oxoindoline-1-carboxylate (9 mg) in the form of a yellow oily substance.

### Example 124

A mixture of 2-(3-methoxy-4-(2-morpholino-2-oxoethoxy)benzylidene)indolin-3-one (Production Example 89; 35 mg), DMF (0.35 ml), and 60% sodium hydride (7.1 mg) was stirred at room temperature for 40 minutes, and methanesulfonic acid anhydride (23 mg) was then added to the reaction mixture under ice cold, followed by stirring the obtained mixture at room temperature for 17 hours.

Thereafter, the solvent was removed from the reaction mixture under a nitrogen flow, and the obtained residue was then purified by silica gel column chromatography (eluent: chloroform-methanol), so as to obtain 2-(3-methoxy-4-(2-morpholino-2-oxoethoxy)benzylidene)-1-(methylsulfonyl)indolin-3-one (9 mg) in the form of a yellow oily substance.

### Example 125

A mixture of 1-acetyl-2-(3-methoxy-4-(2-((tetrahydro-2H-pyran-2-yl)oxy)ethoxy)benzylidene)indolin-3-one (Production Example 195; 123 mg), THF (0.984 ml), water (0.492 ml), and acetic acid (1.968 ml) was stirred at 45° C. for 1 hour 40 minutes. Thereafter, ethyl acetate was added to the reaction mixture, and the thus obtained mixture was then washed with a saturated saline. After that, the organic layer was dried over anhydrous sodium sulfate, and the solvent was then distilled away under reduced pressure.

The obtained residue was purified by silica gel column chromatography (eluent: chloroform-methanol), so as to obtain 1-acetyl-2-(4-(2-hydroxyethoxy)-3-methoxybenzylidene)indolin-3-one (75 mg) in the form of a brown oily substance.

### Example 126

A mixture of (Z)-1-acetyl-2-((6-(2-((tetrahydro-2H-pyran-2-yl)oxy)ethoxy)quinolin-2-yl)methylene)indolin-3-one (Production Example 194; 227 mg), THF (1.8 ml), water (0.9 ml), and acetic acid (3.6 ml) was stirred at 45° C. for 19 hours.

Thereafter, ethyl acetate was added to the reaction mixture, and the thus obtained mixture was then washed with a saturated saline. After that, the organic layer was dried over anhydrous sodium sulfate, and the solvent was then distilled away under reduced pressure. The obtained residue was successively purified by silica gel column chromatography (eluent: chloroform-methanol) and then, by gel permeation chromatography (eluent: chloroform), so as to obtain (Z)-1-acetyl-2-((6-(2-hydroxyethoxy)quinolin-2-yl)methylene)indolin-3-one (36 mg) in the form of a brown oily substance.

### Example 127

TFA (1 ml) was added to a mixture of tert-butyl (Z)-2-((1-acetyl-3-oxoindolin-2-ylidene)methyl)quinoline-6-carboxylate (Production Example 199; 46 mg) and dichloromethane (1 ml) under ice cold, and the obtained mixture was then stirred at 0° C. for 2 hours 30 minutes. Thereafter, dichloromethane was added to the reaction mixture, and the thus obtained mixture was successively washed with water and a saturated saline. After that, the organic layer was dried over anhydrous sodium sulfate, and the solvent was then distilled away under reduced pressure.

The obtained residue was purified by silica gel column chromatography (eluent: chloroform-methanol), so as to obtain (Z)-2-((1-acetyl-3-oxoindolin-2-ylidene)methyl)quinoline-6-carboxylic acid (17 mg) in the form of a brown solid.

### Example 129

TFA (1 ml) was added to a mixture of tert-butyl (Z)-1-(2-((1-acetyl-3-oxoindolin-2-ylidene)methyl)quinoline-6-carbonyl)piperidine-4-carboxylate (Production Example 200; 51 mg), dichloromethane (1 ml), and thioanisole (0.0227 ml) under ice cold TFA (1 ml), and the obtained mixture was then stirred at 0° C. for 4 hours. Thereafter, toluene was added to the reaction mixture, and the solvent was then distilled away under reduced pressure.

The obtained residue was purified by silica gel column chromatography (eluent: chloroform-methanol), so as to obtain (Z)-1-(2-((1-acetyl-3-oxoindolin-2-ylidene)methyl)quinoline-6-carbonyl)piperidine-4-carboxylic acid (42 mg) in the form of a brown oily substance.

### Example 130

TFA (0.46 ml) was added to a mixture of tert-butyl (E)-((2-((1-acetyl-3-oxoindolin-2-ylidene)methyl)benzo[d]thiazol-5-yl)methyl)(tetrahydro-2H-pyran-4-yl)carbamate (Production Example 206; 23 mg) and dichloromethane (0.46 ml) under ice cold, and the obtained mixture was then stirred at 0° C. for 40 minutes.

Thereafter, a saturated sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure.

The obtained residue was purified by silica gel column chromatography (eluent: chloroform-methanol), so as to obtain (E)-1-acetyl-2-((5-(((tetrahydro-2H-pyran-4-yl)amino)methyl)benzo[d]thiazol-2-yl)methylene)indolin-3-one (16 mg) in the form of a light brown solid.

### Example 138

TFA (0.34 ml) was added to a mixture of tert-butyl (Z)-((2-((1-acetyl-3-oxoindolin-2-ylidene)methyl)quinolin-6-yl)methyl)(1-(oxetan-3-yl)piperidin-4-yl)carbamate (Production Example 203; 17 mg) and dichloromethane (0.34 ml) under ice cold, and the obtained mixture was then stirred at 0° C. for 2 hours.

Thereafter, a saturated sodium hydrogen carbonate aqueous solution and sodium hydrogen carbonate were added to the reaction mixture, and a generated product was then extracted with chloroform. After that, the organic layer was dried over anhydrous sodium sulfate, and the solvent was then distilled away under reduced pressure.

The obtained residue was purified by gel permeation chromatography (eluent: chloroform), so as to obtain (Z)-1-acetyl-2-((6-(((1-(oxetan-3-yl)piperidin-4-yl)amino)methyl)quinolin-2-yl)methylene)indolin-3-one (12 mg) in the form of a yellow oily substance.

### Example 139

A mixture of tert-butyl (Z)-4-(2-((1-acetyl-3-oxoindolin-2-ylidene)methyl)quinoline-6-carbonyl)piperazine-1-carboxylate (Production Example 202; 100 mg), 1,4-dioxane (1 ml), and a 4 M hydrogen chloride 1,4-dioxane solution (1 ml) was stirred at room temperature for 1 hour 30 minutes, and the solvent was then distilled away from the reaction mixture under reduced pressure.

Ethyl acetate was added to the obtained residue, and the obtained mixture was then stirred at room temperature for 40 minutes. Thereafter, a generated solid was collected by filtration to obtain 1-acetyl-2-((6-(piperazine-1-carbonyl)quinolin-2-yl)methylene)indolin-3-one dihydrochloride (76 mg) in the form of a brown solid.

### Example 177

A mixture of 4-(1-methyl-1H-pyrazol-4-yl)quinoline-2-carbaldehyde (Production Example 302; 178 mg), 1-acetylindolin-3-one (manufactured by Combi-Blocks; 132 mg), toluene (5 ml), Molecular Sieve 4A (2 g), and piperidine (0.0074 ml) was stirred at 80° C. for 2 hours. Thereafter, the reaction mixture was successively purified by silica gel chromatography (eluent: dichloromethane-methanol) and then, by gel permeation chromatography (eluent: chloroform), and the resultant was then washed with a mixed solvent of diisopropyl ether and chloroform (10:1), so as to obtain (Z)-1-acetyl-2-((4-(1-methyl-1H-pyrazol-4-yl)quinolin-2-yl)methylene)indolin-3-one (77 mg) in the form of a yellow solid.

In Examples 3 to 46, 54, 55, 61 to 97, 100, 101, 112, 113, 116, 118, 128, 131 to 137, 140 to 149, 152, 153, 155 to 159, 162 to 164, 167 to 176, and 178 to 214, the compounds were synthesized according to the above-described methods or methods equivalent thereto. The compound names, structural formulae, double bond forms, synthetic method examples, raw material compounds, and physical property data (1H NMR chemical shift values and MS molecular ion peaks) of the compounds of individual examples will be shown in the following table.

The solvent used in the measurement of 1H NMR is deuterated chloroform, unless otherwise specified.

TABLE 3

| | | | Form | | | |
|---|---|---|---|---|---|---|
| | | | of | | | |
| Ex- | | | double | Synthetic | Raw | |
| ample | | | bonded | method | material | Physical |
| No. | Compound name | Structural formula | portion | Ex. | compound | property data |
| 1 | (Z)-2-(4-((1-acetyl- 3-oxoindolin-2- ylidene)methyl)-2- methoxyphenoxy)- acetamide | 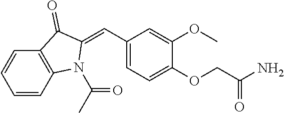 [SMILES: COc1cc(/C=C2/C(=O)c3ccccc3N2C(C)=O)ccc1OCC(N)=O] | Z | | CAS No. 186685- 89-2 Synthetic Literature 8) CAS No. 16800-68-3 | 1H NMR (270 MHz) δ 8.26 (d, J = 8.3 Hz, 1H), 7.87 (dd, J = 7.7, 1.5 Hz, 1H), 7.73-7.63 (m, 1H), 7.37-7.24 |
| | | | | | (Reagent | (m, 2H), 7.17 |
| | | | | | Supplier 3) | (dd, J = 8.3, 2.0 |
| | | | | | | Hz, 1H), 7.12 (d, |
| | | | | | | J = 2.0 Hz, 1H), |
| | | | | | | 6.94 (d, J = 8.3 |
| | | | | | | Hz, 1H), 6.82 (s, |
| | | | | | | 1H), 5.61 (s, 1H), |
| | | | | | | 4.58 (s, 2H), 3.89 |
| | | | | | | (s, 3H), 2.05 (s, |
| | | | | | | 3H). MS 367.03 |
| | | | | | | (M + H) |
| 2 | (Z)-1-acetyl-2-(3- methoxy-4-(2- morpholino-2-oxo- ethoxy)benzyli- dene)indolin-3-one | 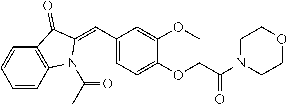 [SMILES: COc1cc(/C=C2/C(=O)c3ccccc3N2C(C)=O)ccc1OCC(=O)N1CCOCC1] | Z | | CAS No. 16800-68-3 (Reagent Supplier 3) CAS No. 31438-76-3 (Reagent | 1H NMR (270 MHz,) δ 8.27 (d, J = 8.3 Hz, 1H), 7.86 (dd, J = 7.6, 1.4 Hz, 1H), 7.74-7.62 ( 1H), 7.51-7.39 |
| | | | | | Supplier 9) | (m, 1H), 7.35- |
| | | | | | | 7.26 (m, 1H), |
| | | | | | | 7.20-7.08 (m, |
| | | | | | | 2H), 6.99 (d, J = |
| | | | | | | 8.3 Hz, 1H), 4.82 |
| | | | | | | (s, 2H), 3.88 (s, |
| | | | | | | 3H), 3.74-3.58 |
| | | | | | | (m, 8H), 2.05 (s, |
| | | | | | | 3H). MS 437.27 |
| | | | | | | (M + H). |
| 3 | 1-acetyl-2-(4- hydroxy-3-meth- oxybenzylidene)- indolin-3-one | 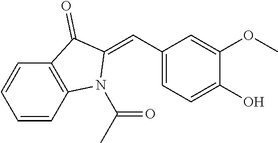 [SMILES: COc1cc(/C=C2/C(=O)c3ccccc3N2C(C)=O)ccc1O] | Z/E Mix. (about 4:1) | Ex. 2 | CAS No. 16800-68-3 (Reagent Supplier 3) CAS No. 121-33-5 (Reagent Supplier 4) | 1H NMR (270 MHz) δ 8.31- 8.12, 7.88-7.78, 7.71-7.60, 7.45- 7.21, 7.17-6.94 (all m, combined 8H), 6.07 (br s, 1H), 4.05, 3.91 (each s, com- |
| | | | | | | bined 3H), 2.63, |
| | | | | | | 2.06 (each s, |
| | | | | | | combined 3H). |
| | | | | | | MS 310.32 |
| | | | | | | (M + H). |
| 4 | 2-(4-((1-acetyl-3- oxoindolin-2-yl- idene)methyl)-2- fluorophenoxy)- acetamide | 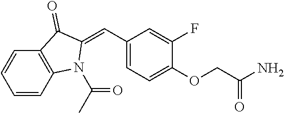 [SMILES: CC(=O)N1/C(=C\c2ccc(OCC(N)=O)c(F)c2)C(=O)c2ccccc21] | Z/E Mix. (about 10:1) | Ex. 2 | CAS No. 1699993- 13-9 (Reagent Supplier 1) CAS No. 16800-68-3 | 1H NMR (270 MHz) δ 8.22 (d, J = 8.2 Hz, 1H), 7.90-7.84 (m, 1H), 7.74-7.66 (m, 1H), 7.37- 7.29 (m, 3H), |
| | | | | | (Reagent | 7.24 (s, 1H), |
| | | | | | Supplier 3) | 7.06-6.97 (m, |
| | | | | | | 1H), 4.61, 4.60 |
| | | | | | | (each s, com- |
| | | | | | | bined 2H), 2.65, |
| | | | | | | 2.10 (each s, |
| | | | | | | combined 3H). |
| | | | | | | MS 355.23 |
| | | | | | | (M + H). |
| 5 | 2-(4-((1-acetyl-3- oxoindolin-2- ylidene)methyl)- phenoxy)acetamide | 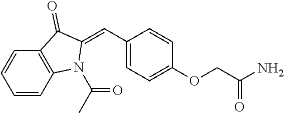 [SMILES: CC(=O)N1/C(=C\c2ccc(OCC(N)=O)cc2)C(=O)c2ccccc21] | Z/E Mix. (about 3:1) | Ex. 2 | CAS No. 16800-68-3 (Reagent Supplier 3) CAS No. 135857- 20-4 | 1H NMR (270 MHz) δ 8.27, 8.10 (each d, each J = 8.3 Hz, com- bined 1H), 8.01- 7.51 (m, 4H), 7.38-7.22 (m, |
| | | | | | (Reagent | 2H), 7.10-6.94 |
| | | | | | Supplier 9) | (m, 2H), 6.54 (br |
| | | | | | | s, 1H), 5.77 (br s, |
| | | | | | | 1H), 4.58, 4.56 |
| | | | | | | (each s, com- |
| | | | | | | bined 2H), 2.65, |
| | | | | | | 2.06 (each s, |
| | | | | | | combined 3H). |
| | | | | | | MS 337.21 |
| | | | | | | (M + H). |
| 6 | 2-(3-((1-acetyl-3- oxoindolin-2-yl- dene)methyl)- phenoxy)acetamide | 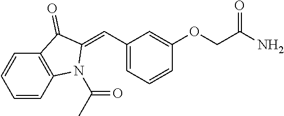 [SMILES: CC(=O)N1/C(=C\c2cccc(OCC(N)=O)c2)C(=O)c2ccccc21] | Z/E Mix. (about 2:1) | Ex. 2 | CAS No. 16800-68-3 (Reagent Supplier 3) CAS No. 849015- 95-8 (Reagent | 1H NMR (270 MHz) δ 8.27, 8.07 (each d, each J = 8.4 Hz, com- bined 1H), 7.87, 7.78 (each dd, each J = 7.6, 1.5 Hz, combined |
| | | | | | Supplier 15) | 1H), 7.74-6.91 |
| | | | | | | (m, 7H), 6.57 (br |
| | | | | | | s, 1H), 5.84 (br |
| | | | | | | s, 1H), 4.60, 4.52 |
| | | | | | | (each s, com- |
| | | | | | | bined 2H), 2.66, |
| | | | | | | 1.99 (each s, |
| | | | | | | combined 3H). |
| | | | | | | MS 337.22 |
| | | | | | | (M + H). |
| 7 | (Z)-1-acetyl-2-(3- methoxy-4-(2- morpholinoethoxy)- benzylidene)indolin- 3-one | 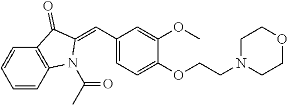 [SMILES: COc1cc(/C=C2/C(=O)c3ccccc3N2C(C)=O)ccc1OCCN1CCOCC1] | Z | Ex. 2 | CAS No. 16800-68-3 (Reagent Supplier 3) CAS No. 46995-89-5 (Reagent | 1H NMR (270 MHzMHz) δ 8.28 (d, J = 8.3 Hz, 1H), 7.86 (dd, J = 7.5, 1.5 Hz, 1H), 7.72- 7.62 (m, 1H), |
| | | | | | Supplier 5) | 7.35-7.25 (m, |
| | | | | | | 2H), 7.16 (dd, |
| | | | | | | J = 8.3, 2.1 Hz, |
| | | | | | | 1H), 7.08 (d, J = |
| | | | | | | 2.1 Hz, 1H), |
| | | | | | | 6.96-6.91 (m, |
| | | | | | | 1H), 4.28-4.14 |
| | | | | | | (m, 2H), 3.87 (s, |
| | | | | | | 3H), 3.80-3.69 |
| | | | | | | (m, 4H), 2.95- |
| | | | | | | 2.81 (m, 2H), |
| | | | | | | 2.70-2.55 (m, |
| | | | | | | 4H), 2.05 (s, 3H). |
| | | | | | | MS 423.29 |
| | | | | | | (M + H). |
| 8 | 2-(2-((1-acetyl-3- oxoindolin-2-yl- idene)methyl)- phenoxy)acetamide | 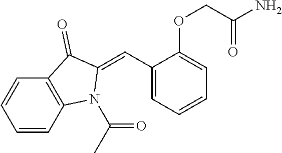 [SMILES: CC(=O)N1/C(=C\c2ccccc2OCC(N)=O)C(=O)c2ccccc21] | Z/E Mix. (about 3:1) | Ex. 2 | CAS No. 16800-68-3 (Reagent Supplier 3) CAS No. 24590-06-5 (Reagent Supplier 8) | 1H NMR (270 MHz) δ 8.29- 7.60 (m, 3H), 7.53-7.21 (m, 4H), 7.16-7.03 (m, 1H), 6.97, 6.88 (each d, each J = 8.4 Hz, combined 1H), 6.54 (br s, 1H), |
| | | | | | | 5.92 (br s, 1H), |
| | | | | | | 4.57, 4.55 (each |
| | | | | | | s, combined 2H), |
| | | | | | | 2.68, 1.94 (each |
| | | | | | | s, combined 3H). |
| | | | | | | MS 337.19 |
| | | | | | | (M + H). |
| 9 | 2-(4-((1-acetyl-3- oxoindolin-2-yl- idene)methyl)-2- methoxyphenoxy)- acetonitrile | 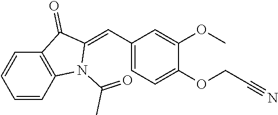 [SMILES: COc1cc(/C=C2/C(=O)c3ccccc3N2C(C)=O)ccc1OCC#N] | Z/E Mix (about 3:2) | Ex. 2 | CAS No. 16800-68-3 (Reagent Supplier 3) CAS No. 342592- 62-5 (Reagent | 1H NMR (270 MHz) δ 8.31- 7.53 (m, 4H), 7.38-7.04 (m, 4H), 4.90, 4.89 (each s, com- bined 2H), 4.02, 3.91 (each s, |
| | | | | | Supplier 13) | combined 3H), |
| | | | | | | 2.65, 2.04 (each |
| | | | | | | s, combined 3H). |
| | | | | | | MS 349.25 |
| | | | | | | (M + H). |
| 10 | 1-acetyl-2-(4- (benzyloxy)-3- methoxybenz- ylidene)indolin- 3-one | 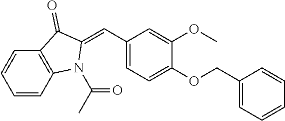 [SMILES: COc1cc(/C=C2/C(=O)c3ccccc3N2C(C)=O)ccc1OCc1ccccc1] | Z/E Mix. (about 10:1) | Ex. 2 | CAS No. 16800-68-3 (Reagent Supplier 3) CAS No. 2426-87-1 (Reagent Supplier 4) | 1H NMR (270 MHz) δ 8.28 (d, J = 8.3 Hz, 1H), 7.86 (dd, J = 7.7, 1.4 Hz, 1H), 7.71-7.62 (m, 1H), 7.50-7.07 (m, 9H), 6.99- |
| | | | | | | 6.90 (m, 1H), |
| | | | | | | 5.25, 5.21 (each |
| | | | | | | s, combined 2H), |
| | | | | | | 4.04, 3.90 (each |
| | | | | | | s, combined 3H), |
| | | | | | | 2.62, 2.04 (each |
| | | | | | | s, combined 3H). |
| | | | | | | MS 400.21 |
| | | | | | | (M + H). |
| 11 | 1-acetyl-2-(3- methoxy-4-(2- methoxyethoxy)- benzylidene)- indolin-3-one | 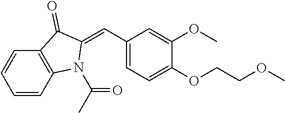 [SMILES: COCCOc1ccc(/C=C2/C(=O)c3ccccc3N2C(C)=O)cc1OC] | Z/E Mix. (about 2:1) | Ex. 2 | CAS No. 16800-68-3 (Reagent Supplier 3) CAS No. 114991- 70-7 | 1H NMR (270 MHz) δ 8.29, 8.15 (each d, each J = 8.5 Hz, combined 1H), 8.10-7.04 (m, 6H), 6.98-6.92 |
| | | | | | (Reagent | (m, 1H), 4.29- |
| | | | | | Supplier 15) | 4.18 (m, 2H), |
| | | | | | | 4.00, 3.87 (each |
| | | | | | | s, combined 3H), |
| | | | | | | 3.86-3.78 (m, |
| | | | | | | 2H), 3.46 (s, 3H), |
| | | | | | | 2.63, 2.04 (each |
| | | | | | | s, combined 3H). |
| | | | | | | MS 368.20 |
| | | | | | | (M + H). |
| 12 | 1-acetyl-2-(3- methoxy-4-(2- phenoxyethoxy)- benzylidene)- indolin-3-one | 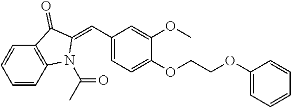 [SMILES: COc1cc(/C=C2/C(=O)c3ccccc3N2C(C)=O)ccc1OCCOc1ccccc1] | Z/E Mix. (about 2:1) | Ex. 2 | CAS No. 16800-68-3 (Reagent Supplier 3) CAS No. 299936- 09-7 | 1H NMR (270 MHz) δ 8.33- 8.12 (m, 1H), 8.09-6.91 (m, 12H), 4.49-4.36 (m, 4H), 4.00, 3.87 (each s, |
| | | | | | (Reagent | combined 3H), |
| | | | | | Supplier 6) | 2.64, 2.05 (each |
| | | | | | | s, combined 3H). |
| | | | | | | MS 430.22 |
| | | | | | | (M + H). |
| 13 | 2-(4-((1-acetyl-3- oxoindolin-2- ylidene)methyl)- 2-ethoxyphenoxy)- acetamide | 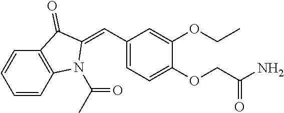 [SMILES: CCOc1cc(/C=C2/C(=O)c3ccccc3N2C(C)=O)ccc1OCC(N)=O] | Z/E Mix. (about 3:1) | Ex. 2 | CAS No. 16800-68-3 (Reagent Supplier 3) CAS No. 346724- 04-7 | 1H NMR (270 MHz) δ 8.32- 8.08 (m, 1H), 8.07-6.88 (m, 8H), 6.03 (br s, 1H), 4.60, 4.57 (each s,com- |
| | | | | | (Reagent | bined 2H), 4.26, |
| | | | | | Supplier 16) | 4.09 (each q, |
| | | | | | | each J = 6.9 Hz, |
| | | | | | | combined 2H), |
| | | | | | | 2.64, 2.04 (each |
| | | | | | | s, combined 3H), |
| | | | | | | 1.57-1.43 (m, |
| | | | | | | 3H). MS 381.12 |
| | | | | | | (M + H). |
| 14 | 1-acetyl-2-(3- methoxy-4-(2- oxo-2-(pyrroli- din-1-yl)ethoxy)- benzylidene)- indolin-3-one | 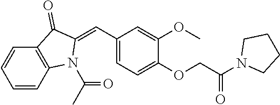 [SMILES: COc1cc(/C=C2/C(=O)c3ccccc3N2C(C)=O)ccc1OCC(=O)N1CCCC1] | Z/E Mix. (about 4:1) | Ex. 2 | CAS No. 16800-68-3 (Reagent Supplier 3) CAS No. 760183- 53-7 | 1H NMR (270 MHz) δ 8.33- 8.08 (m, 1H), 7.91-7.76 (m, 1H), 7.73-7.59 (m, 1H), 7.46- 7.23 (m, 2H), |
| | | | | | (Reagent | 7.18-7.00 (m, |
| | | | | | Supplier 17) | 2H), 6.99-6.91 |
| | | | | | | (m, 1H), 4.77, |
| | | | | | | 4.75 (each s, |
| | | | | | | combined 2H), |
| | | | | | | 4.02, 3.89 (each |
| | | | | | | s, combined 3H), |
| | | | | | | 3.59-3.47 (m, |
| | | | | | | 4H), 2.63, 2.05 |
| | | | | | | (each s, com- |
| | | | | | | bined 3H), 2.03- |
| | | | | | | 1.82 (m, 4H). MS |
| | | | | | | 421.27 (M + H). |
| 15 | 2-(4-((1-acetyl-3- oxoindolin-2-yl- idene)methyl)-2- methoxyphenoxy)- N-phenylacetamide | 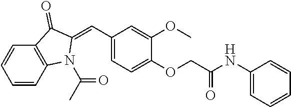 [SMILES: COc1cc(/C=C2/C(=O)c3ccccc3N2C(C)=O)ccc1OCC(=O)Nc1ccccc1] | Z/E Mix. (about 10:1) | Ex. 2 | CAS No. 16800-68-3 (Reagent Supplier 3) CAS No. 31438-73-0 (Reagent | 1H NMR (270 MHz) δ 8.73 (br s, 1H), 8.31-8.05 (m, 1H), 7.90- 7.77 (m, 1H), 7.73-7.56 (m, 3H), 7.43-7.28 |
| | | | | | Supplier 18) | (m, 3H), 7.22- |
| | | | | | | 7.12 (m, 3H), |
| | | | | | | 7.02 (d, J = 8.3 |
| | | | | | | Hz, 1H), 4.72, |
| | | | | | | 4.69 (each s, |
| | | | | | | combined 2H), |
| | | | | | | 4.08, 3.96 (each |
| | | | | | | s, combined |
| | | | | | | 3H), 2.65, 2.06 |
| | | | | | | (each s, com- |
| | | | | | | bined 3H). MS |
| | | | | | | 443.28 (M + H). |
| 16 | 1-acetyl-2-(3- fluoro-4-(2- morpholino-2- oxoethoxy)- benzylidene)- indolin-3-one | 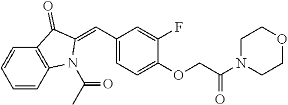 [SMILES: CC(=O)N1/C(=C\c2ccc(OCC(=O)N3CCOCC3)c(F)c2)C(=O)c2ccccc21] | Z/E Mix. (about 4:5) | Ex. 2 | CAS No. 1920634- 06-5 (Reagent Supplier 1) CAS No. 16800-68-3 | 1H NMR (270 MHz) δ 8.27- 7.50 (m, 5H), 7.36-7.21 (m, 2H), 7.15-7.02 (m, 1H), 4.83 (s, 2H), 3.76-3.56 |
| | | | | | (Reagent | (m, 8H), 2.64, |
| | | | | | Supplier 3) | 2.09 (each s, |
| | | | | | | combined 3H). |
| | | | | | | MS 425.22 |
| | | | | | | (M + H). |
| 17 | 2-(4-((1-acetyl-3- oxoindolin-2-yl- idene)methyl)-2- chlorophenoxy)- acetamide | 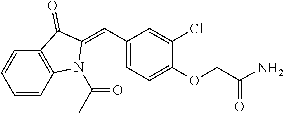 [SMILES: CC(=O)N1/C(=C\c2ccc(OCC(N)=O)c(Cl)c2)C(=O)c2ccccc21] | Z/E Mix. (about 2:1) | Ex. 2 | CAS No. 16800-68-3 (Reagent Supplier 3) CAS No. 333743- 26-3 | 1H NMR (270 MHz) δ 8.26- 7.76 (m, 3H), 7.74-7.17 (m, 4H), 7.01-6.90 (m, 1H), 6.79 (br s, 1H), 6.21 |
| | | | | | (Reagent | (br s, 1H), 4.61, |
| | | | | | Supplier 19) | 4.60 (each s, |
| | | | | | | combined 2H), |
| | | | | | | 2.65, 2.10 (each |
| | | | | | | s, combined 3H). |
| | | | | | | MS 371.14 |
| | | | | | | (M + H). |
| 18 | 1-acetyl-2-(4-(2- morpholino-2- oxoethoxy)-3- nitrobenzyli- dene)indolin-3- one | 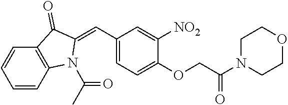 [SMILES: CC(=O)N1/C(=C\c2ccc(OCC(=O)N3CCOCC3)c([N+](=O)[O-])c2)C(=O)c2ccccc21] | Z/E Mix. (about 3:2) | Ex. 2 | CAS No. 1097009- 40-9 (Reagent Supplier 1) CAS No. 16800-68-3 | 1H NMR (270 MHz) δ 8.53- 8.35, 8.11-7.60 (each m, com- bined 5H), 7.41- 7.17 (m, 3H), 4.91 (s, 2H), |
| | | | | | (Reagent | 3.77-3.58 (m, |
| | | | | | Supplier 3) | 8H), 2.67, 2.25 |
| | | | | | | (each s, com- |
| | | | | | | bined 3H). MS |
| | | | | | | 452.19 (M + H). |
| 19 | 1-acetyl-2-(3,5- difluoro-4-(2- morpholino-2- oxoethoxy)- benzylidene)- indolin-3-one | 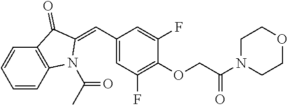 [SMILES: CC(=O)N1/C(=C\c2cc(F)c(OCC(=O)N3CCOCC3)c(F)c2)C(=O)c2ccccc21] | Z/E Mix. (about 5:4) | Ex. 2 | CAS No. 1537434- 02-8 (Reagent Supplier 1) CAS No. 16800-68-3 | 1H NMR (270 MHz) δ 8.23- 7.94 (m, 1H), 7.91-7.76 (m, 1H), 7.76-7.61 (m, 1H), 7.60- 7.03 (m, 4H), |
| | | | | | (Reagent | 4.91, 4.90 (each |
| | | | | | Supplier 3) | s, combined 2H), |
| | | | | | | 3.81-3.52 (m, |
| | | | | | | 8H), 2.64, 2.12 |
| | | | | | | (each s, com- |
| | | | | | | bined 3H). MS |
| | | | | | | 443.18 (M + H). |
| 20 | (2E)-3-(4-((1- acetyl-3-oxo- indolin-2-yl- idene)methyl)-2- methoxyphen- yl)acrylamide | 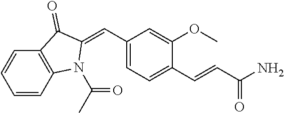 [SMILES: COc1cc(/C=C2/C(=O)c3ccccc3N2C(C)=O)ccc1/C=C/C(N)=O] | Z/E Mix. (about 3:1) | Ex. 2 | Production Ex. 3 CAS No. 16800- 68-3 (Reagent Supplier 3) | 1H NMR (270 MHz) δ 8.32- 8.04 (m, 1H), 7.96-7.03 (m, 8H), 6.72-6.60 (m, 1H), 5.75 (br s, 2H), 4.00, |
| | | | | | | 3.90 (each s, |
| | | | | | | combined 3H), |
| | | | | | | 2.66, 2.03 (each |
| | | | | | | s, combined 3H). |
| | | | | | | MS 363.24 |
| | | | | | | (M + H). |
| 21 | 4-((1-acetyl-3- oxoindolin-2- ylidene)methyl)- 2-methoxyphenyl methanesulfonate | 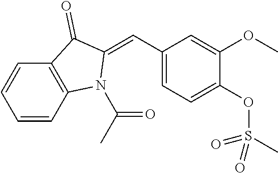 [SMILES: COc1cc(/C=C2/C(=O)c3ccccc3N2C(C)=O)ccc1OS(C)(=O)=O] | Z/E Mix. (about 5:2) | Ex. 2 | CAS No. 52200-05-2 (Reagent Supplier 1) CAS No. 16800-68-3 (Reagent Supplier 3) | 1H NMR (270 MHz) δ 8.28- 8.21, 8.09-8.02, 7.73-7.59 (all m, combined 3H), 7.87, 7.80 (each dd, each J = 7.7, 1.4 Hz, combined 1H), 7.41-7.11 (m, 4H), 4.02, 3.91 |
| | | | | | | (each s, com- |
| | | | | | | bined 3H), 3.24, |
| | | | | | | 3.23 (each s, |
| | | | | | | combined 3H), |
| | | | | | | 2.65, 2.02 (each |
| | | | | | | s, combined 3H). |
| | | | | | | MS 388.18 |
| | | | | | | (M + H). |
| 22 | (Z)-1-acetyl-2- ((5-(2-morph- olino-2-oxo- ethoxy)pyridin- 2-yl)methylene)- indolin-3-one | 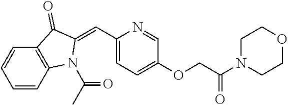 [SMILES: CC(=O)N1/C(=C\c2ccc(OCC(=O)N3CCOCC3)cn2)C(=O)c2ccccc21] | Z | Ex. 2 | CAS No. 1511998- 57-4 (Reagent Supplier 1) CAS No. 16800-68-3 | 1H NMR (500 MHz) δ 8.43 (d, J = 3.0 Hz, 1H), 8.21-8.17 (m, 1H), 7.83-7.79 (m, 1H), 7.65- 7.60 (m, 1H), |
| | | | | | (Reagent | 7.49 (d, J = 8.6 |
| | | | | | Supplier 3) | Hz, 1H), 7.31 |
| | | | | | | (dd, J = 8.6, 3.0 |
| | | | | | | Hz, 1H), 7.25- |
| | | | | | | 7.20 (m, 1H), |
| | | | | | | 7.19 (s, 1H), |
| | | | | | | 4.80 (s, 2H), |
| | | | | | | 3.73-3.67 (m, |
| | | | | | | 4H), 3.67-3.61 |
| | | | | | | (m, 2H), 3.58- |
| | | | | | | 3.53 (m, 2H), |
| | | | | | | 2.10 (s, 3H). MS |
| | | | | | | 408.21 (M + H). |
| 23 | 2-(4-((1-acetyl-3- oxoindolin-2-yl- idene)methyl)-3- methoxyphenoxy)- acetamide | 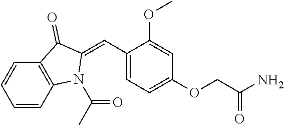 [SMILES: COc1cc(OCC(N)=O)ccc1/C=C1/C(=O)c2ccccc2N1C(C)=O] | Z/E Mix. (about 4:1) | Ex. 2 | CAS No. 883794- 26-1 (Reagent Supplier 1) CAS No. 16800-68-3 (Reagent | 1H NMR (270 MHz) δ 8.34- 8.15, 7.70-7.58, 7.50-7.42, 7.33- 7.20 (all m, com- bined 5H), 7.85, 7.76 (each dd, each J = 7.6, 1.5 |
| | | | | | Supplier 3) | Hz, combined |
| | | | | | | 1H), 6.65-6.47 |
| | | | | | | (m, 3H), 6.27 |
| | | | | | | (br s, 1H), 4.58, |
| | | | | | | 4.56 (each s, |
| | | | | | | combined 2H), |
| | | | | | | 3.88, 3.86 (each |
| | | | | | | s, combined 3H), |
| | | | | | | 2.62, 1.98 (each |
| | | | | | | s, combined 3H). |
| | | | | | | MS 367.12 |
| | | | | | | (M + H). |
| 24 | 1-acetyl-2-(3- methoxy-4- ((methylsulfon- yl)methoxy)- benzylidene)- indolin-3-one | 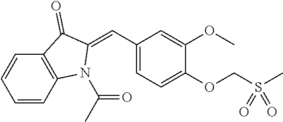 [SMILES: COc1cc(/C=C2/C(=O)c3ccccc3N2C(C)=O)ccc1OCS(C)(=O)=O] | Z/E Mix. (about 5:2) | Ex. 2 | Production Ex. 8 CAS No. 16800-68-3 (Reagent Supplier 3) | 1H NMR (270 MHz) δ 8.29- 8.23, 8.13-7.99, 7.73-7.61, 7.55- 7.52 (all m, com- bined 3H), 7.86, 7.81 (each dd, each J = 7.6, 1.5 |
| | | | | | | Hz, combined |
| | | | | | | 1H), 7.38-7.09 |
| | | | | | | (m, 4H), 5.07, |
| | | | | | | 5.05 (each s, |
| | | | | | | combined 2H), |
| | | | | | | 4.01, 3.90 (each |
| | | | | | | s, combined |
| | | | | | | 3H), 3.07 (s, |
| | | | | | | 3H), 2.64, 2.03 |
| | | | | | | (each s, com- |
| | | | | | | bined 3H). MS |
| | | | | | | 402.18 (M + H). |
| 25 | 1-acetyl-2-(3- methoxy-4- ((methylsul- finyl)meth- oxy)benzyl- idene)indolin- 3-one | 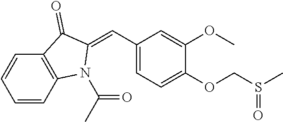 [SMILES: COc1cc(/C=C2/C(=O)c3ccccc3N2C(C)=O)ccc1OCS(C)=O] | Z/E Mix. (about 4:1) | Ex. 2 | Production Ex. 9 CAS No. 16800-68-3 (Reagent Supplier 3) | 1H NMR (270 MHz) δ 8.29- 8.22, 8.13-8.03, 7.73-7.61, 7.55- 7.44 (all m, combined 3H), 7.86, 7.81 (each dd, each J = 7.7, |
| | | | | | | 1.5 Hz, com- |
| | | | | | | bined 1H), 7.36- |
| | | | | | | 7.08 (m, 4H), |
| | | | | | | 5.17-4.95 (m, |
| | | | | | | 2H), 4.01, 3.89 |
| | | | | | | (each s, com- |
| | | | | | | bined 3H), 2.75 |
| | | | | | | (s, 3H), 2.65, |
| | | | | | | 2.04 (each s, |
| | | | | | | combined 3H). |
| | | | | | | MS 386.17 |
| | | | | | | (M + H). |
| 26 | 2-(4-((1-acetyl-3- oxoindolin-2-yl- idene)methyl)-3,5-dimethoxyphen- oxy)acetamide | 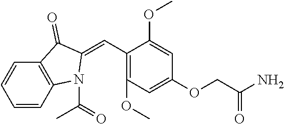 [SMILES: COc1cc(OCC(N)=O)cc(OC)c1/C=C1/C(=O)c2ccccc2N1C(C)=O] | Z/E Mix. (about 5:1) | Ex. 2 | Production Ex. 11 CAS No. 16800-68-3 (Reagent Supplier 3) | 1H NMR (270 MHz) δ 8.37- 8.23 (m, 1H), 7.88-7.68 (m, 1H), 7.67-7.56 (m, 1H), 7.52- 7.50, 7.28-7.15 (each m, com- |
| | | | | | | bined 2H), 6.53 |
| | | | | | | (br s, 1H), 6.18, |
| | | | | | | 6.16 (each s, |
| | | | | | | combined 2H), |
| | | | | | | 5.76 (br s, 1H), |
| | | | | | | 4.57 (s, 2H), |
| | | | | | | 3.83, 3.81 (each |
| | | | | | | s, combined 6H), |
| | | | | | | 2.65, 1.88 (each |
| | | | | | | s, combined 3H). |
| | | | | | | MS 397.29 |
| | | | | | | (M + H). |
| 27 | N-(4-((1-acetyl-3- oxoindolin-2-yl- idene)methyl)-2- methoxyphenyl)- 2-morpholino- acetamide | 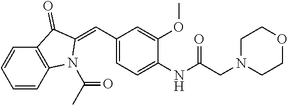 [SMILES: COc1cc(/C=C2/C(=O)c3ccccc3N2C(C)=O)ccc1NC(=O)CN1CCOCC1] | Z/E Mix. (about 2:1) | Ex. 2 | Production Ex. 16 CAS No. 16800-68-3 (Reagent Supplier 3) | 1H NMR (270 MHz) δ 10.02, 9.98 (each br s, combined 1H), 8.54-8.42 (m, 1H), 8.33-8.22 (m, 1H), 7.91- |
| | | | | | | 7.78 (m, 1H), |
| | | | | | | 7.73-7.59 (m, |
| | | | | | | 1H), 7.53-7.42, |
| | | | | | | 7.35-7.18, 7.10- |
| | | | | | | 7.07 (all m, |
| | | | | | | combined 4H), |
| | | | | | | 4.08, 3.94 (each |
| | | | | | | s, combined 3H), |
| | | | | | | 3.87-3.75 (m, |
| | | | | | | 4H), 3.19 (s, |
| | | | | | | 2H), 2.71-2.60 |
| | | | | | | (m, 4H), 2.65, |
| | | | | | | 2.05 (each s, |
| | | | | | | combined 3H). |
| | | | | | | MS 436.38 |
| | | | | | | (M + H). |
| 28 | (Z)-1-acetyl-2- ((6-(morpholine- 4-carbonyl)quin- olin-2-yl)methyl- ene)indolin-3-one | 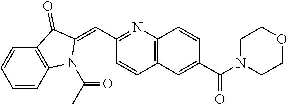 [SMILES: CC(=O)N1/C(=C\c2ccc3cc(C(=O)N4CCOCC4)ccc3n2)C(=O)c2ccccc21] | Z | Ex. 2 | Production Ex. 119 CAS No. 16800- 68-3 (Reagent Supplier 3) | 1H NMR (500 MHz) δ 8.23 (d, J = 8.6 Hz, 1H), 8.18 (d, J = 8.2 Hz, 1H), 8.14 (d, J = 8.6 Hz, 1H), 7.91 (d, J = 1.8 |
| | | | | | | Hz, 1H), 7.83 |
| | | | | | | (dd, J = 7.6, 1.5 |
| | | | | | | Hz, 1H), 7.74 |
| | | | | | | (dd, J = 8.7, 1.9 |
| | | | | | | Hz, 1H), 7.68- |
| | | | | | | 7.62 (m, 2H), |
| | | | | | | 7.33 (s, 1H), |
| | | | | | | 7.27-7.22 (m, |
| | | | | | | 1H), 3.94-3.42 |
| | | | | | | (m, 8H), 2.13 (s, |
| | | | | | | 3H). MS 428.53 |
| | | | | | | (M + H). |
| 29 | 1-acetyl-2-((6-(2- morpholino-2- oxoethoxy)-[1,1′- biphenyl]-3-yl)- methylene)indo- lin-3-one | 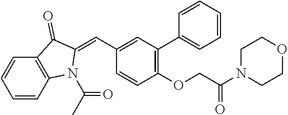 [SMILES: CC(=O)N1/C(=C\c2ccc(OCC(=O)N3CCOCC3)c(-c3ccccc3)c2)C(=O)c2ccccc21] | Z/E Mix. (about 2:1) | Ex. 2 | Production Ex. 51 CAS No. 16800-68-3 (Reagent Supplier 3) | 1H NMR (500 MHz) δ 8.26- 8.22, 8.14-8.11, 7.97-7.92, 7.71- 7.23 (all m, combined 11H), 7.85, 7.80 (each |
| | | | | | | dd, each J = 7.6, |
| | | | | | | 1.5 Hz, com- |
| | | | | | | bined 1H), 7.08- |
| | | | | | | 7.02 (m, 1H), |
| | | | | | | 4.74, 4.73 (each |
| | | | | | | s, combined 2H), |
| | | | | | | 3.63-3.55 (m, |
| | | | | | | 4H), 3.37-3.25 |
| | | | | | | (m, 4H), 2.63, |
| | | | | | | 2.13 (each s, |
| | | | | | | combined 3H). |
| | | | | | | MS 483.35 |
| | | | | | | (M + H). |
| 30 | 2-(4-((1-acetyl-3- oxoindolin-2- ylidene)methyl)- 2-isopropoxy- phenoxy)acet- amide | 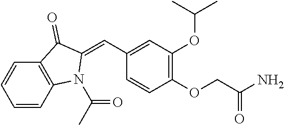 [SMILES: CC(=O)N1/C(=C\c2ccc(OCC(N)=O)c(OC(C)C)c2)C(=O)c2ccccc21] | Z/E Mix. (about 3:1) | Ex. 2 | Production Ex. 10 CAS No. 16800-68-3 (Reagent Supplier 3) | 1H NMR (270 MHz) δ 8.27, 8.11 (each d, each J = 8.3 Hz, combined 1H), 8.04 (d, J = 2.1 Hz, 0.3H), 7.86, 7.81 (each dd, |
| | | | | | | each J = 7.7, |
| | | | | | | 1.4 Hz, com- |
| | | | | | | bined 1H), 7.73- |
| | | | | | | 7.60 (m, 1H), |
| | | | | | | 7.51-6.88 (m, |
| | | | | | | 5.7H), 6.00 (br |
| | | | | | | s, 1H), 4.80-4.49 |
| | | | | | | (m, 3H), 2.64, |
| | | | | | | 2.06 (each s, |
| | | | | | | combined 3H), |
| | | | | | | 1.46, 1.39 (each |
| | | | | | | d, each J = 6.0 |
| | | | | | | Hz, combined |
| | | | | | | 6H). MS 395.31 |
| | | | | | | (M + H). |
| 31 | 3-(4-((1-acetyl-3- oxoindolin-2-yl- idene)methyl)-2- methoxyphenyl) [symbol] | 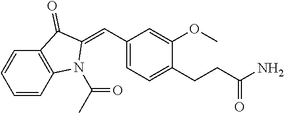 [SMILES: COc1cc(/C=C2/C(=O)c3ccccc3N2C(C)=O)ccc1CCC(N)=O] | Z/E Mix. (about 2:1) | Ex. 2 | Production Ex. 6 CAS No. 16800-68-3 (Reagent Supplier 3) | 1H NMR (270 MHz) δ 8.33- 8.09 (m, 1H), 7.89-7.76 (m, 1H), 7.72-7.60 (m, 1H), 7.52- 6.97 (m, 5H), |
| | | | | | | 5.55 (br s, 2H), |
| | | | | | | 3.96, 3.84 (each |
| | | | | | | s, combined 3H), |
| | | | | | | 3.07-2.92 (m, |
| | | | | | | 2H), 2.64, 1.99 |
| | | | | | | (each s, com- |
| | | | | | | bined 3H), 2.60- |
| | | | | | | 2.48 (m, 2H). |
| | | | | | | MS 365.31 |
| | | | | | | (M + H). |
| 32 | (Z)-1-acetyl-2- ((6-fluoro-5-(2- morpholino-2- oxoethoxy)- pyridin-2-yl)- methylene)- indolin-3-one | 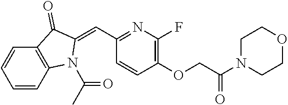 [SMILES: CC(=O)N1/C(=C\c2ccc(OCC(=O)N3CCOCC3)c(F)n2)C(=O)c2ccccc21] | Z | Ex. 2 | Production Ex. 52 CAS No. 16800-68-3 (Reagent Supplier 3) | 1H NMR (270 MHz) δ 8.17 (d, J = 8.5 Hz, 1H), 7.81 (dd, J = 7.7, 1.4 Hz, 1H), 7.68- 7.60 (m, 1H), 7.49-7.36 (m, |
| | | | | | | 2H), 7.29-7.19 |
| | | | | | | (m, 1H), 7.11 (s, |
| | | | | | | 1H), 4.86 (s, 2H), |
| | | | | | | 3.76-3.54 (m, |
| | | | | | | 8H), 2.20 (s, 3H). |
| | | | | | | MS 426.38 |
| | | | | | | (M + H). |
| 33 | (Z)-1-acetyl-2- ((6-(hydroxymeth- yl)quinolin-2-yl)- methylene)indolin- 3-one | 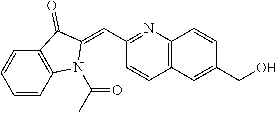 [SMILES: CC(=O)N1/C(=C\c2ccc3cc(CO)ccc3n2)C(=O)c2ccccc21] | Z | Ex. 2 | Production Ex. 120 CAS No. 16800- 68-3 (Reagent Supplier 3) | 1H NMR (270 MHz) δ 8.23- 8.14 (m, 2H), 8.08 (d, J = 8.7 Hz, 1H), 7.87- 7.78 (m, 2H), 7.76-7.55 (m, |
| | | | | | | 3H), 7.34-7.21 |
| | | | | | | (m, 2H), 4.91 (s, |
| | | | | | | 2H), 2.11 (s, 3H). |
| | | | | | | MS 345.32 |
| | | | | | | (M + H). |
| 34 | (Z)-2-((1-acetyl-3- oxoindolin-2-yl- idene)methyl)quin- oline-6-carbonitrile | 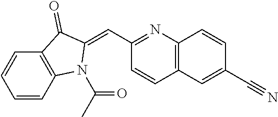 [SMILES: CC(=O)N1/C(=C\c2ccc3cc(C#N)ccc3n2)C(=O)c2ccccc21] | Z | Ex. 2 | CAS No. 904885- 93-4 (Reagent Supplier 1) CAS No. 16800-68-3 (Reagent | 1H NMR (270 MHz) δ 8.28-8.11 (m, 4H), 7.91- 7.82 (m, 2H), 7.72-7.63 (m, 2H), 7.31 (s, 1H), 7.30-7.22 (m, 1H), 2.16 (s, 3H). |
| | | | | | Supplier 3) | MS 340.33 |
| | | | | | | (M + H). |
| 35 | (Z)-1-acetyl-2- ((6-(morpholino- methyl)quinolin- 2-yl)methylene)- indolin-3-one | 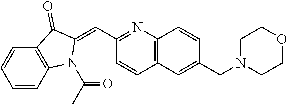 [SMILES: CC(=O)N1/C(=C\c2ccc3cc(CN4CCOCC4)ccc3n2)C(=O)c2ccccc21] | Z | Ex. 2 | Production Ex. 121 CAS No. 16800- 68-3 (Reagent Supplier 3) | 1H NMR (270 MHz) δ 8.21 (d, J = 8.3 Hz, 1H), 8.16 (d, J = 8.5 Hz, 1H), 8.06 (d, J = 8.6 Hz, 1H), 7.84 (dd, J = 7.3, |
| | | | | | | 1.5 Hz, 1H), |
| | | | | | | 7.81-7.71 (m, |
| | | | | | | 2H), 7.69-7.62 |
| | | | | | | (m, 1H), 7.59 (d, |
| | | | | | | J = 8.4 Hz, 1H), |
| | | | | | | 7.35 (s, 1H), |
| | | | | | | 7.28-7.21 (m, |
| | | | | | | 1H), 3.78-3.70 |
| | | | | | | (m, 4H), 3.68 (s, |
| | | | | | | 2H), 2.55-2.47 |
| | | | | | | (m, 4H), 2.13 (s, |
| | | | | | | 3H). MS 414.45 |
| | | | | | | (M + H). |
| 36 | (E)-1-acetyl-2- ((5-(morpholine- 4-carbonyl)-1H- indol-2-yl)meth- ylene)indolin-3- one | 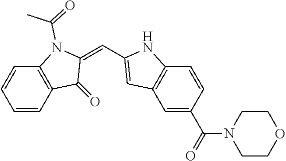 [SMILES: CC(=O)N1/C(=C/c2cc3cc(C(=O)N4CCOCC4)ccc3[nH]2)C(=O)c2ccccc21] | E | Ex. 2 | Production Ex. 192 CAS No. 16800- 68-3 (Reagent Supplier 3) | 1H NMR (270 MHz) δ 12.83 (br s, 1H), 8.30 (s, 1H), 7.94 (dd, J = 7.6, 1.5 Hz, 1H), 7.86 (d, J = 8.5 Hz, 1H), 7.76-7.66 (m, 2H), 7.53 (d, J = 8.4 Hz, 1H), |
| | | | | | | 7.41-7.29 (m, |
| | | | | | | 2H), 7.05-7.01 |
| | | | | | | (m, 1H), 3.85- |
| | | | | | | 3.47 (m, 8H), |
| | | | | | | 2.73 (s, 3H). MS |
| | | | | | | 416.25 (M + H). |
| 37 | 1-acetyl-2-((6- (morpholine-4- carbonyl)-1H- indol-3-yl)meth- ylene)indolin-3- one | 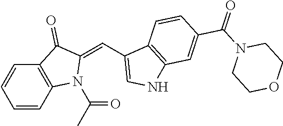 [SMILES: CC(=O)N1/C(=C\c2c[nH]c3cc(C(=O)N4CCOCC4)ccc23)C(=O)c2ccccc21] | Z/E Mix. (about 2:3) | Ex. 2 | Production Ex. 42 CAS No. 16800- 68-3 (Reagent Supplier 3) | 1H NMR (500 MHz) δ 11.02, 10.74 (each br s, combined 1H), 9.41, 8.31 (each s, combined 1H), 8.26, 8.01 (each d, each J = 8.3 |
| | | | | | | Hz, combined |
| | | | | | | 1H), 7.89-7.84 |
| | | | | | | (m, 1H), 7.79 (d, |
| | | | | | | J = 8.3 Hz, 1H), |
| | | | | | | 7.68-7.54 (m, |
| | | | | | | 3H), 7.33-7.20 |
| | | | | | | (m, 2H), 3.97- |
| | | | | | | 3.37 (m, 8H), |
| | | | | | | 2.71, 2.11 (each |
| | | | | | | s, combined 3H). |
| | | | | | | MS 416.34 |
| | | | | | | (M + H). |
| 38 | (Z)-1-acetyl-2- ((5-(morpholine- 4-carbonyl)thia- zol-2-yl)meth- ylene)indolin-3- one | 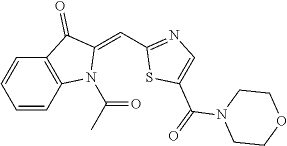 [SMILES: CC(=O)N1/C(=C\c2ncc(C(=O)N3CCOCC3)s2)C(=O)c2ccccc21] | Z | Ex. 2 | Production Ex. 187 CAS No. 16800- 68-3 (Reagent Supplier 3) | 1H NMR (270 MHz) δ 8.16- 8.09 (m, 2H), 7.83 (dd, J = 7.4, 1.4 Hz, 1H), 7.71-7.62 (m, 1H), 7.32-7.24 (m, 2H), 3.79- 3.73 (m, 8H), |
| | | | | | | 2.26 (s, 3H). MS |
| | | | | | | 384.22 (M + H). |
| 39 | (Z)-1-acetyl-2- ((1-methylsulfon- yl)-1H-pyrrolo [3,2-b]pyridin-5- yl)methylene)- indolin-3-one | 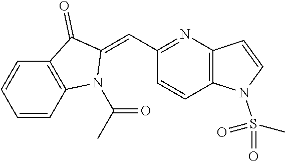 [SMILES: CC(=O)N1/C(=C\c2ccc3c(ccn3S(C)(=O)=O)n2)C(=O)c2ccccc21] | Z | Ex. 2 | Production Ex. 57 CAS No. 16800- 68-3 (Reagent Supplier 3) | 1H NMR (270 MHz) δ 8.26- 8.18 (m, 2H), 7.84 (dd, J = 7.5, 1.4 Hz, 1H), 7.77 (d, J = 3.7 Hz, 1H), 7.70-7.61 (m, 1H), 7.55 (d, J = 8.6 Hz, 1H), 7.35 (s, 1H), |
| | | | | | | 7.29-7.21 (m, |
| | | | | | | 1H), 6.96 (dd, |
| | | | | | | J = 3.9, 0.9 Hz, |
| | | | | | | 1H), 3.21 (s, |
| | | | | | | 3H), 2.10 (s, 3H). |
| | | | | | | MS 382.31 |
| | | | | | | (M + H). |
| 40 | (Z)-1-acetyl-2-((2- (morpholine-4- carbonyl)-1H-pyr- rolo[3,2-b]pyridin- 5-yl)methylene)- indolin-3-one | 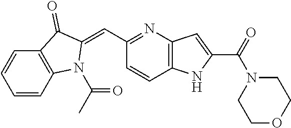 [SMILES: CC(=O)N1/C(=C\c2ccc3[nH]c(C(=O)N4CCOCC4)cc3n2)C(=O)c2ccccc21] | Z | Ex. 2 | Production Ex. 190 CAS No. 16800- 68-3 (Reagent Supplier 3) | 1H NMR (270 MHz) δ 9.75 (s, 1H), 8.24 (d, J = 8.3 Hz, 1H), 7.84 (dd, J = 7.6, 1.4 Hz, 1H), 7.79 (dd, J = 8.5, 0.9 Hz, 1H), 7.68- |
| | | | | | | 7.60 (m, 1H), |
| | | | | | | 7.50 (d, J = 8.5 |
| | | | | | | Hz, 1H), 7.37 (s, |
| | | | | | | 1H), 7.28-7.20 |
| | | | | | | (m, 1H), 6.99 |
| | | | | | | (dd, J = 2.3, 0.9 |
| | | | | | | Hz, 1H), 4.05- |
| | | | | | | 3.74 (m, 8H), |
| | | | | | | 2.09 (s, 3H). MS |
| | | | | | | 417.36 (M + H). |
| 41 | (Z)-1-acetyl-2- ((6-(4-(oxetan-3- yl)piperazine-1- carbonyl)quino- lin-2-yl)methyl- ene)indolin-3-one | 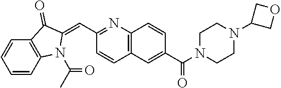 [SMILES: CC(=O)N1/C(=C\c2ccc3cc(C(=O)N4CCN(C5COC5)CC4)ccc3n2)C(=O)c2ccccc21] | Z | Ex. 2 | Production Ex. 135 CAS No. 16800- 68-3 (Reagent | 1H NMR (270 MHz) δ 8.25-8.20 (m, 1H), 8.18 (d, J = 8.3 Hz, 1H), 8.14 (d, J = 8.7 Hz, 1H), 7.90 (d, |
| | | | | | Supplier 3) | J = 1.7 Hz, 1H), |
| | | | | | | 7.85 (dd, J = 7.5, |
| | | | | | | 1.4 Hz, 1H), 7.74 |
| | | | | | | (dd, J = 8.7, 1.9 |
| | | | | | | Hz, 1H), 7.70- |
| | | | | | | 7.61 (m, 2H), |
| | | | | | | 7.33 (s, 1H), |
| | | | | | | 7.29-7.21 (m, |
| | | | | | | 1H), 4.73-4.58 |
| | | | | | | (m, 4H), 3.98- |
| | | | | | | 3.77 (m, 2H), |
| | | | | | | 3.66-3.43 (m, |
| | | | | | | 3H), 2.53-2.22 |
| | | | | | | (m, 4H), 2.13 (s, |
| | | | | | | 3H). MS 483.43 |
| | | | | | | (M + H). |
| 42 | (Z)-1-acetyl-2- ((6-(4-(2-hydroxy- 3-methoxyprop- yl)piperazine-1- carbonyl)quin- | 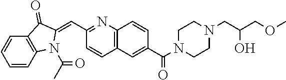 [SMILES: COCC(O)CN1CCN(C(=O)c2ccc3nc(/C=C4/C(=O)c5ccccc5N4C(C)=O)ccc3c2)CC1] | Z | Ex. 2 | Production Ex. 136 CAS No. 16800- 68-3 | 1H NMR (270 MHz) δ 8.26-8.16 (m, 2H), 8.14 (d, J = 8.6 Hz, 1H), 7.89 (d, J = 1.9 |
| | olin-2-yl)methyl- | | | | (Reagent | Hz, 1H), 7.85 (dd, |
| | ene)indolin-3-one | | | | Supplier 3) | J = 7.7, 1.4 Hz, |
| | | | | | | 1H), 7.74 (dd, J = |
| | | | | | | 8.7, 1.9 Hz, 1H), |
| | | | | | | 7.71-7.61 (m, |
| | | | | | | 2H), 7.33 (s, 1H), |
| | | | | | | 7.30-7.22 (m, |
| | | | | | | 1H), 3.98-3.04 |
| | | | | | | (m, 10H), 2.81- |
| | | | | | | 2.29 (m, 6H), |
| | | | | | | 2.13 (s, 3H). MS |
| | | | | | | 515.51 (M + H). |
| 43 | (E)-1-acetyl-2- ((6-((E)-2-mor- pholinosulfonyl)- vinyl)benzo[d] thiazol-2-yl)meth- ylene)indolin-3- one | 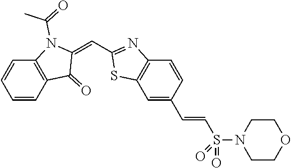 [SMILES: CC(=O)N1/C(=C/c2nc3ccc(/C=C/S(=O)(=O)N4CCOCC4)cc3s2)C(=O)c2ccccc21] | E | Ex. 2 | Production Ex. 137 CAS No. 16800- 68-3 (Reagent Supplier 3) | 1H NMR (270 MHz) δ 8.36 (s, 1H), 8.15-8.03 (m, 3H), 7.94 (dd, J = 7.7, 1.5 Hz, 1H), 7.78- 7.59 (m, 3H), 7.39-7.32 (m, 1H), 6.79 (d, J = 15.5 Hz, 1H), 3.84-3.77 (m, |
| | | | | | | 4H), 3.28-3.20 |
| | | | | | | (m, 4H), 2.77 (s, |
| | | | | | | 3H). MS 496.36 |
| | | | | | | (M + H). |
| 44 | (Z)-1-acetyl-2- ((6-(2,6-dimethyl- morpholine-4- carbonyl)quin- olin-2-yl)methyl- ene)indolin-3-one | 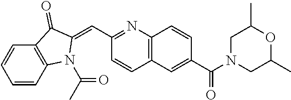 [SMILES: CC(=O)N1/C(=C\c2ccc3cc(C(=O)N4CC(C)OC(C)C4)ccc3n2)C(=O)c2ccccc21] | Z | Ex. 2 | Production Ex. 144 CAS No. 16800- 68-3 (Reagent | 1H NMR (270 MHz) δ 8.26-8.12 (m, 3H), 7.90 (d, J = 1.9 Hz, 1H), 7.87-7.82 (m, 1H), 7.76-7.62 |
| | | | | | Supplier 3) | (m, 3H), 7.34 (s, |
| | | | | | | 1H), 7.29-7.22 |
| | | | | | | (m, 1H), 5.01- |
| | | | | | | 3.41 (m, 6H), |
| | | | | | | 2.15 (s, 3H), |
| | | | | | | 1.35-1.03 (m, |
| | | | | | | 6H). MS 456.40 |
| | | | | | | (M + H). |
| 45 | (Z)-1-acetyl-2- ((6-(3-morpho- lino-3-oxoprop- yl)quinolin-2-yl)- methylene)indolin- 3-one | 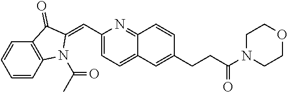 [SMILES: CC(=O)N1/C(=C\c2ccc3cc(CCC(=O)N4CCOCC4)ccc3n2)C(=O)c2ccccc21] | Z | Ex. 2 | Production Ex. 158 CAS No. 16800- 68-3 (Reagent | 1H NMR (270 MHz) δ 8.21 (d, J = 8.3 Hz, 1H), 8.13 (d, J = 8.4 Hz, 1H), 8.04 (d, J = 8.5 Hz, 1H), |
| | | | | | Supplier 3) | 7.84 (dd, J = 7.6, |
| | | | | | | 1.5 Hz, 1H), |
| | | | | | | 7.75-7.51 (m, |
| | | | | | | 4H), 7.34 (s, 1H), |
| | | | | | | 7.29-7.20 (m, |
| | | | | | | 1H), 3.67-3.61 |
| | | | | | | (m, 4H), 3.59- |
| | | | | | | 3.52 (m, 2H), |
| | | | | | | 3.46-3.36 (m, |
| | | | | | | 2H), 3.19 (t, J = |
| | | | | | | 7.6 Hz, 2H), 2.73 |
| | | | | | | (t, J = 7.6 Hz, |
| | | | | | | 2H), 2.11 (s, 3H). |
| | | | | | | MS 456.40 |
| | | | | | | (M + H). |
| 46 | (Z)-1-acetyl-2- ((6′-(morpholine- 4-carbonyl)-[3,4′- biquinolin]-2′- yl)methylene)- indolin-3-one | 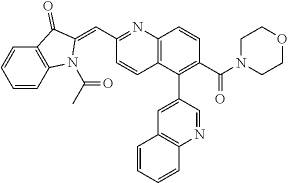 [SMILES: CC(=O)N1/C(=C\c2ccc3c(-c4cnc5ccccc5c4)c(C(=O)N4CCOCC4)ccc3n2)C(=O)c2ccccc21] | Z | Ex. 2 | Production Ex. 175 CAS No. 16800- 68-3 (Reagent Supplier 3) | 1H NMR (500 MHz) δ 9.05 (d, J = 2.2 Hz, 1H), 8.34 (dd, J = 2.3, 0.8 Hz, 1H), 8.26-8.22 (m, 2H), 8.19-8.16 (m, 1H), 7.96- 7.92 (m, 1H), 7.91 (dd, J = 1.9, 0.6 Hz, 1H), |
| | | | | | | 7.89-7.82 (m, |
| | | | | | | 2H), 7.78 (dd, J = |
| | | | | | | 8.6, 1.8 Hz, 1H), |
| | | | | | | 7.71-7.64 (m, |
| | | | | | | 3H), 7.36 (s, 1H), |
| | | | | | | 7.28-7.21 (m, |
| | | | | | | 1H), 3.84-3.36 |
| | | | | | | (m, 8H), 2.23 (s, |
| | | | | | | 3H). MS 555.38 |
| | | | | | | (M + H) |
| 47 | (Z)-1-acetyl-2- ((5-(morpholine- 4-carbonyl)benzo [d]thiazol-2-yl)- methylene)- indolin-3-one | 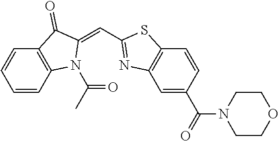 [SMILES: CC(=O)N1/C(=C\c2nc3cc(C(=O)N4CCOCC4)ccc3s2)C(=O)c2ccccc21] | Z | | Production Ex. 131 CAS No. 16800- 68-3 (Reagent Supplier 3) | 1H NMR (270 MHz) δ 8.18 (d, J = 8.3 Hz, 1H), 8.13 (d, J = 1.6 Hz, 1H), 7.99 (d, J = 8.3 Hz, 1H), 7.85 (dd, J = 7.7, 1.4 Hz, 1H), 7.73-7.64 (m, |
| | | | | | | 1H), 7.53 (dd, J = |
| | | | | | | 8.3, 1.6 Hz, 1H), |
| | | | | | | 7.34-7.14 (m, |
| | | | | | | 2H), 3.90-3.36 |
| | | | | | | (m, 8H), 2.26 (s, |
| | | | | | | 3H). MS 434.37 |
| | | | | | | (M + H). |
| 48 | (E)-1-acetyl-2- ((5-(morpholine- 4-carbonyl)benzo [d]thiazol-2-yl)- methylene)indolin- 3-one | 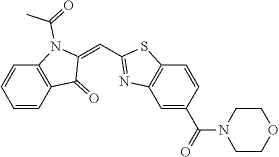 [SMILES: CC(=O)N1/C(=C/c2nc3cc(C(=O)N4CCOCC4)ccc3s2)C(=O)c2ccccc21] | E | | Production Ex. 131 CAS No. 16800- 68-3 (Reagent Supplier 3) | 1H NMR (270 MHz) δ 8.34 (s, 1H), 8.11 (d, J = 1.5 Hz, 1H), 8.09-8.00 (m, 2H), 7.92 (dd, J = 7.5, 1.4 Hz, 1H), 7.76-7.68 (m, 1H), 7.56 (dd, J = 8.2, 1.6 Hz, 1H), |
| | | | | | | 7.38-7.30 (m, |
| | | | | | | 1H), 3.98-3.42 |
| | | | | | | (m, 8H), 2.76 (s, |
| | | | | | | 3H). MS 434.35 |
| | | | | | | (M + H). |
| 49 | 2-(4-((1-benzoyl- 3-oxoindolin-2- ylidene)methyl)- 2-methoxyphen- oxy)acetamide | 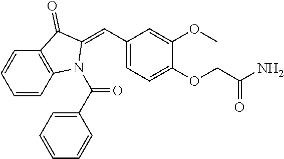 [SMILES: COc1cc(/C=C2/C(=O)c3ccccc3N2C(=O)c2ccccc2)ccc1OCC(N)=O] | Z/E Mix. (about 2:1) | | CAS No. 186685- 89-2 (Synthetic Literature 8) CAS No. 106698- 08-2 (Synthetic Literature 9) | 1H NMR (270 MHz) δ 8.23- 7.69 (m, 2H), 7.65-7.06 (m, 9H), 6.97-6.62 (m, 3H), 5.86 (br s, 1H), 4.57, 4.50 (each s, combined 2H), 4.00, 3.71 (each |
| | | | | | | s, combined 3H). |
| | | | | | | MS 429.17 |
| | | | | | | (M + H). |
| 50 | 1-acetyl-5-fluoro- 2-(3-methoxy-4- (2-morpholino-2- oxoethoxy)benz- ylidene)indolin-3- one | 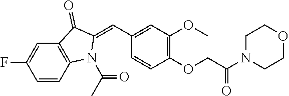 [SMILES: COc1cc(/C=C2/C(=O)c3cc(F)ccc3N2C(C)=O)ccc1OCC(=O)N1CCOCC1] | Z/E Mix. (about 2:1) | | CAS No. 3802-82-2 (Reagent Supplier 1) CAS No. 31438-76-3 | 1H NMR (270 MHz) δ 8.28, 8.23 (each dd, each J = 9.0, 4.1 Hz, combined 1H), 8.10-8.06, |
| | | | | | (Reagent | 7.53-7.29 (each |
| | | | | | Supplier 9) | m, combined |
| | | | | | | 4H), 7.18-6.96 |
| | | | | | | (m, 2H), 4.86, |
| | | | | | | 4.82 (each s, |
| | | | | | | combined 2H), |
| | | | | | | 4.01, 3.88 (each |
| | | | | | | s, combined 3H), |
| | | | | | | 3.72-3.59 (m, |
| | | | | | | 8H), 2.61, 2.04 |
| | | | | | | (each s, com- |
| | | | | | | bined 3H). MS |
| | | | | | | 455.36 (M + H). |
| 51 | 1-acetyl-2-(3- methoxy-4-(2- morpholino-2- oxoethoxy)benz- ylidene)-1,2- dihydro-3H-pyr- rolo[2,3-b]pyridin- | 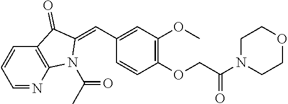 [SMILES: COc1cc(/C=C2/C(=O)c3cccnc3N2C(C)=O)ccc1OCC(=O)N1CCOCC1] | Z/E Mix. (about 3:4) | | CAS No. 155818- 89-6 (Reagent Supplier 1) CAS No. 31438-76-3 | 1H NMR (270 MHz) δ 8.66- 8.58, 8.05-8.02, 7.47-7.18, 7.01- 6.92 (all m, com- bined 6H), 8.16, 8.11 (each dd, |
| | 3-one | | | | (Reagent | each J = 7.6, 1.9 |
| | | | | | Supplier 9) | Hz, combined |
| | | | | | | 1H), 4.82, 4.80 |
| | | | | | | (each s, combined |
| | | | | | | 2H), 3.97, 3.88 |
| | | | | | | (each s, combined |
| | | | | | | 3H), 3.72-3.60 |
| | | | | | | (m, 8H), 2.97, |
| | | | | | | 2.87 (each s, com- |
| | | | | | | bined 3H). MS |
| | | | | | | 438.36 (M + H). |
| 52 | (1E)-2-(4-((1-acet- yl-3-oxoindolin-2- ylidene)methyl)-2- methoxyphenyl)- ethene-1-sulfon- amide | 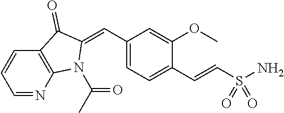 [SMILES: COc1cc(/C=C2/C(=O)c3cccnc3N2C(C)=O)ccc1/C=C/S(N)(=O)=O] | Z/E Mix. (about 5:4) | | Production Ex. 5 CAS No. 16800- 68-3 (Reagent Supplier 3) | 1H NMR (270 MHz) δ 8.29- 8.22, 8.08-8.01, 7.92-7.57, 7.52- 7.40, 7.37-7.05 (all m, combined 10H), 4.69 (s, |
| | | | | | | 2H), 4.02, 3.93 |
| | | | | | | (each s, combined |
| | | | | | | 3H), 2.67, 2.03 |
| | | | | | | (each s, combined |
| | | | | | | 3H). MS 399.14 |
| | | | | | | (M + H). |
| 53 | (E)-N-((2-((1-acet- yl-3-oxoindolin-2- ylidene)methyl)- benzo[d]thiazol-5- yl)methyl)meth- anesulfonamide | 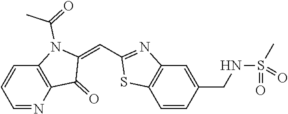 [SMILES: CC(=O)N1/C(=C/c2nc3cc(CNS(C)(=O)=O)ccc3s2)C(=O)c2ncccc21] | E | | Production Ex. 141 CAS No. 16800- 68-3 (Reagent Supplier 3) | 1H NMR (270 MHz, dimethyl- sulfoxide-d6) δ 8.51 (s, 1H), 8.20 (d, J = 8.3 Hz, 1H), 8.09-8.03 (m, 2H), 7.92- |
| | | | | | | 7.79 (m, 2H), |
| | | | | | | 7.71 (t, J = 6.2 |
| | | | | | | Hz, 1H), 7.54 |
| | | | | | | (dd, J = 8.3, 1.7 |
| | | | | | | Hz, 1H), 7.41 (t, |
| | | | | | | J = 7.3 Hz, 1H), |
| | | | | | | 4.36 (d, J = 6.3 |
| | | | | | | Hz, 2H), 2.91 (s, |
| | | | | | | 3H), 2.72 (s, 3H). |
| | | | | | | MS 428.34 |
| | | | | | | (M + H). |
| 54 | (Z)-6-((1-acetyl-3- oxoindolin-2-yl- idene)methyl)-2- fluoropyridin-3-yl methanesulfonate | 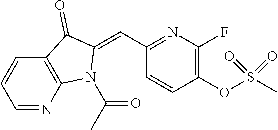 [SMILES: CC(=O)N1/C(=C\c2ccc(OS(C)(=O)=O)c(F)n2)C(=O)c2cccnc21] | Z | Ex. 53 | Production Ex. 54 CAS No. 16800- 68-3 (Reagent Supplier 3) | 1H NMR (270 MHz, dimethyl- sulfoxide-d6) δ 8.19 (dd, J = 9.8, 8.1 Hz, 1H), 8.07 (d, J = 8.3 Hz, 1H), 7.93 (d, J = 8.1 Hz, 1H), |
| | | | | | | 7.83-7.72 (m, |
| | | | | | | 2H), 7.34 (t, J = |
| | | | | | | 7.3 Hz, 1H), 7.25 |
| | | | | | | (s, 1H), 3.60 |
| | | | | | | (s, 3H), 2.12 (s, |
| | | | | | | 3H). MS 377.23 |
| | | | | | | (M + H). |
| 55 | (E)-1-acetyl-2-((4- (morpholine-4- carbonyl)thiazol- 2-yl)methylene)- indolin-3-one | 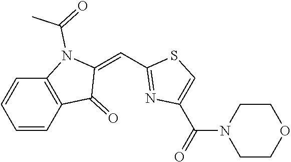 [SMILES: CC(=O)N1/C(=C/c2nc(C(=O)N3CCOCC3)cs2)C(=O)c2ccccc21] | E | Ex. 53 | Production Ex 185 CAS No. 16800- 68-3 (Reagent Supplier 3) | 1H NMR (270 MHz, dimethyl- sulfoxide-d6) δ 8.50 (s, 1H), 8.35 (s, 1H), 8.04 (d, J = 8.4 Hz, 1H), 7.90 (d, J = 7.6 Hz, 1H), 7.83 (t, J = 7.8 Hz, 1H), 7.41 (t, J = 7.5 |
| | | | | | | Hz, 1H), 3.81- |
| | | | | | | 3.55 (m, 8H), |
| | | | | | | 2.70 (s, 3H). MS |
| | | | | | | 384.17 (M + H). |
| 56 | (E)-N-((2-((1- acetyl-3-oxoin- dolin-2-ylidene)- methyl)benzo[d] thiazol-6-yl)meth- yl)methanesulfon- amide | 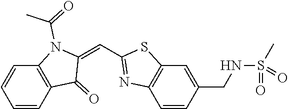 [SMILES: CC(=O)N1/C(=C/c2nc3ccc(CNS(C)(=O)=O)cc3s2)C(=O)c2ccccc21] | E | | Production Ex. 145 CAS No. 16800- 68-3 (Reagent Supplier 3) | 1H NMR (270 MHz, dimethyl- sulfoxide-d6) δ 8.50 (s, 1H), 8.15 (d, J = 1.6 Hz, 1H), 8.11-8.04 (m, 2H), 7.90 |
| | | | | | | (dd, J = 7.6, 1.5 |
| | | | | | | Hz, 1H), 7.87- |
| | | | | | | 7.79 (m, 1H), |
| | | | | | | 7.71 (t, J = 6.3 |
| | | | | | | Hz, 1H), 7.58 |
| | | | | | | (dd, J = 8.5, 1.7 |
| | | | | | | Hz, 1H), 7.41 (t, |
| | | | | | | J = 7.4 Hz, 1H), |
| | | | | | | 4.35 (d, J = 6.4 |
| | | | | | | Hz, 2H), 2.92 (s, |
| | | | | | | 3H), 2.72 (s, 3H). |
| | | | | | | MS 428.26 |
| | | | | | | (M + H). |
| 57 | 2-(4-((1-acetyl-3- oxoindolin-2-yl- idene)methyl)-2- methoxyphenoxy)- acetic acid | 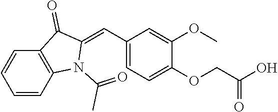 [SMILES: COc1cc(/C=C2/C(=O)c3ccccc3N2C(C)=O)ccc1OCC(=O)O] | Z/E Mix. (about 5:2) | | CAS No. 16800-68-3 (Reagent Supplier 3) CAS No. 1660-19-1 (Reagent | 1H NMR (270 MHz) δ 8.25- 7.55 (m, 3H), 7.45-6.72 (m, 5H), 4.65, 4.58 (each s, com- bined 2H), 3.87, |
| | | | | | Supplier 9) | 3.81 (each s, |
| | | | | | | combined 3H), |
| | | | | | | 2.59, 2.01 (each |
| | | | | | | s, combined 3H). |
| | | | | | | MS 368.19 |
| | | | | | | (M + H). |
| 58 | (E)-1-acetyl-2- ((6-(morpholine- 4-carbonyl)benzo [d]thiazol-2-yl)- methylene)indolin- 3-one | 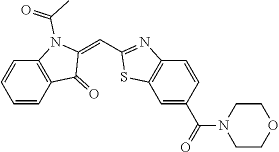 [SMILES: CC(=O)N1/C(=C/c2nc3ccc(C(=O)N4CCOCC4)cc3s2)C(=O)c2ccccc21] | E | | Production Ex. 126 CAS No. 16800-68-3 (Reagent Supplier 3) | 1H NMR (270 MHz) δ 8.33 (s, 1H), 8.13 (d, J = 8.3 Hz, 1H), 8.10-8.04 (m, 2H), 7.96-7.90 (m, 1H), 7.77- 7.69 (m, 1H), 7.57 (dd, J = 8.5, 1.6 Hz, 1H), 7.35 |
| | | | | | | (t, J = 7.4 Hz, |
| | | | | | | 1H), 3.91-3.55 |
| | | | | | | (m, 8H), 2.77 (s, |
| | | | | | | 3H). MS 434.08 |
| | | | | | | (M + H). |
| 59 | (Z)-1-acetyl-2- ((6-(morpholine- 4-carbonyl)benzo [d]thiazol-2-yl)- methylene)indo- lin-3-one | 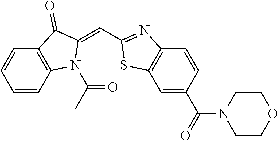 [SMILES: CC(=O)N1/C(=C\c2nc3ccc(C(=O)N4CCOCC4)cc3s2)C(=O)c2ccccc21] | Z | | Production Ex. 126 CAS No. 16800-68-3 (Reagent Supplier 3) | 1H NMR (270 MHz) δ 8.19 (d, J = 8.4 Hz, 1H), 8.13 (d, J = 8.4 Hz, 1H), 8.02 (d, J = 1.7 Hz, 1H), 7.85 (dd, J = 7.6, 1.3 Hz, 1H), 7.73-7.64 (m, |
| | | | | | | 1H), 7.57 (dd, |
| | | | | | | J = 8.6, 1.6 Hz, |
| | | | | | | 1H), 7.32 (s, |
| | | | | | | 1H), 7.31-7.26 |
| | | | | | | (m, 1H), 3.98- |
| | | | | | | 3.35 (m, 8H), |
| | | | | | | 2.25 (s, 3H). MS |
| | | | | | | 434.11 (M + H). |
| 60 | (Z)-1-acetyl-2- ((6-(1,1-dioxido- thiomorpholine-4- carbonyl)quinolin- 2-yl)methylene)- indolin-3-one | 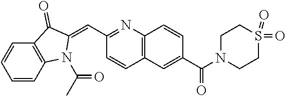 [SMILES: CC(=O)N1/C(=C\c2ccc3cc(C(=O)N4CCS(=O)(=O)CC4)ccc3n2)C(=O)c2ccccc21] | Z | | Production Ex. 132 CAS No. 16800-68-3 (Reagent Supplier 3) | 1H NMR (500 MHz) δ 8.24 (dd, J = 8.6, 0.9 Hz, 1H), 8.20-8.14 (m, 2H), 7.95 (d, J = 1.9 Hz, 1H), |
| | | | | | | 7.86-7.83 (m, |
| | | | | | | 1H), 7.74 (dd, J = |
| | | | | | | 8.6, 1.9 Hz, 1H), |
| | | | | | | 7.69-7.64 (m, |
| | | | | | | 2H), 7.33 (s, 1H), |
| | | | | | | 7.28-7.24 (m, |
| | | | | | | 1H), 4.39-3.95 |
| | | | | | | (m, 4H), 3.28- |
| | | | | | | 2.96 (m, 4H), |
| | | | | | | 2.15 (s, 3H). MS |
| | | | | | | 476.33 (M + H). |
| 61 | (Z)-6-((1-acetyl- 3-oxoindolin-2- ylidene)methyl)- pyridin-3-yl methanesulfonate | 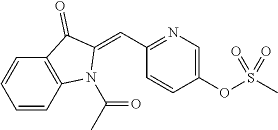 [SMILES: CC(=O)N1/C(=C\c2ccc(OS(C)(=O)=O)cn2)C(=O)c2ccccc21] | Z | Ex. 60 | Production Ex. 55 CAS No. 16800-68-3 (Reagent Supplier 3) | 1H NMR (270 MHz) δ 8.65 (d, J = 2.7 Hz, 1H), 8.16 (d, J = 8.4 Hz, 1H), 7.83 (dd, J = 7.6, 1.6 Hz, 1H), 7.72 (dd, J = 8.6, 2.7 |
| | | | | | | Hz, 1H), 7.69- |
| | | | | | | 7.61 (m, 1H), |
| | | | | | | 7.59 (d, J = 8.6 |
| | | | | | | Hz, 1H), 7.29- |
| | | | | | | 7.21 (m, 1H), |
| | | | | | | 7.20 (s, 1H), 3.26 |
| | | | | | | (s, 3H), 2.12 (s, |
| | | | | | | 3H). MS 359.30 |
| | | | | | | (M + H). |
| 62 | (Z)-1-acetyl-2- ((7-(morpholine- 4-carbonyl)quino- lin-2-yl)methyl-ene)indolin-3-one | 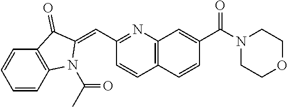 [SMILES: CC(=O)N1/C(=C\c2ccc3ccc(C(=O)N4CCOCC4)cc3n2)C(=O)c2ccccc21] | Z | Ex. 60 | Production Ex. 122 CAS No. 16800-68-3 (Reagent Supplier 3) | 1H NMR (270 MHz) δ 8.23 (d, J = 8.4 Hz, 1H), 8.18 (d, J = 8.2 Hz, 1H), 8.08 (d, J = 1.5 Hz, 1H), 7.93-7.81 (m, |
| | | | | | | 2H), 7.71-7.61 |
| | | | | | | (m, 3H), 7.33 (s, |
| | | | | | | 1H), 7.29-7.21 |
| | | | | | | (m, 1H), 3.99- |
| | | | | | | 3.40 (m, 8H), |
| | | | | | | 2.13 (s, 3H). MS |
| | | | | | | 428.41 (M + H). |
| 63 | (Z)-N-((2-((1- acetyl-3-oxoin- dolin-2-ylidene)- methyl)quinolin- 6-yl)methyl)- acetamide | 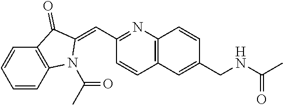 [SMILES: CC(=O)NCc1ccc2nc(/C=C3/C(=O)c4ccccc4N3C(C)=O)ccc2c1] | Z | Ex. 60 | Production Ex. 123 CAS No. 16800-68-3 (Reagent Supplier 3) | 1H NMR (270 MHz) δ 8.19 (d, J = 8.2 Hz, 1H), 8.12 (d, J = 8.4 Hz, 1H), 8.03 (d, J = 8.6 Hz, 1H), 7.83 (dd, J = 7.7, |
| | | | | | | 1.5 Hz, 1H), |
| | | | | | | 7.70-7.60 (m, |
| | | | | | | 3H), 7.55 (d, J = |
| | | | | | | 8.4 Hz, 1H), 7.29 |
| | | | | | | (s, 1H), 7.28-7.20 |
| | | | | | | (m, 1H), 6.17 (br |
| | | | | | | s, 1H), 4.61 (d, |
| | | | | | | J = 5.9 Hz, 2H), |
| | | | | | | 2.10 (s, 3H), 2.09 |
| | | | | | | (s, 3H). MS |
| | | | | | | 386.40 (M + H). |
| 64 | (Z)-N-((2-((1- acetyl-3-oxoin- dolin-2-ylidene)- methyl)quinolin- 6-yl)methyl)meth- anesulfonamide | 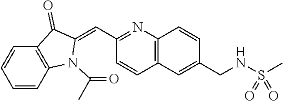 [SMILES: CC(=O)N1/C(=C\c2ccc3cc(CNS(C)(=O)=O)ccc3n2)C(=O)c2ccccc21] | Z | Ex. 60 | Production Ex. 124 CAS No. 16800-68-3 (Reagent Supplier 3) | 1H NMR (270 MHz) δ 8.22- 8.15 (m, 2H), 8.11 (d, J = 8.7 Hz, 1H), 7.84 (dd, J = 7.6, 1.4 Hz, 1H), 7.80 (d, |
| | | | | | | J = 1.9 Hz, 1H), |
| | | | | | | 7.74-7.58 (m, |
| | | | | | | 3H), 7.32 (s, 1H), |
| | | | | | | 7.29-7.21 (m, |
| | | | | | | 1H), 4.96-4.86 |
| | | | | | | (m, 1H), 4.53 (d, |
| | | | | | | J = 6.3 Hz, 2H), |
| | | | | | | 2.95 (s, 3H), 2.13 |
| | | | | | | (s, 3H). MS |
| | | | | | | 422.43 (M + H). |
| 65 | (Z)-1-acetyl-2- ((6-(2-morpho- lino-2-oxoethoxy)- quinolin-2-yl)meth- ylene)indolin-3- one | 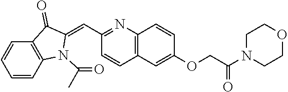 [SMILES: CC(=O)N1/C(=C\c2ccc3cc(OCC(=O)N4CCOCC4)ccc3n2)C(=O)c2ccccc21] | Z | Ex. 60 | Production Ex. 125 CAS No. 16800-68-3 (Reagent Supplier 3) | 1H NMR (270 MHz) δ 8.20 (d, J = 8.2 Hz, 1H), 8.10 (d, J = 8.5 Hz, 1H), 8.04 (d, J = 9.3 Hz, 1H), |
| | | | | | | 7.84 (dd, J = 7.8, |
| | | | | | | 1.4 Hz, 1H), |
| | | | | | | 7.69-7.61 (m, |
| | | | | | | 1H), 7.57 (d, J = |
| | | | | | | 8.5 Hz, 1H), 7.43 |
| | | | | | | (dd, J = 9.2, 2.8 |
| | | | | | | Hz, 1H), 7.33 (s, |
| | | | | | | 1H), 7.28-7.20 |
| | | | | | | (m, 1H), 7.17 (d, |
| | | | | | | J = 2.9 Hz, 1H), |
| | | | | | | 4.86 (s, 2H), |
| | | | | | | 3.75-3.60 (m, |
| | | | | | | 8H), 2.12 (s, 3H). |
| | | | | | | MS 458.47 |
| | | | | | | (M + H). |
| 66 | 1-acetyl-2-((6- (morpholine-4- carbonyl)naph- thalen-2-yl)meth- ylene)indolin-3- one | 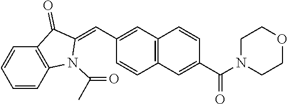 [SMILES: CC(=O)N1/C(=C\c2ccc3cc(C(=O)N4CCOCC4)ccc3c2)C(=O)c2ccccc21] | Z/E Mix. (about 4:3) | Ex. 60 | Production Ex. 188 CAS No. 16800-68-3 (Reagent Supplier 3) | 1H NMR (270 MHz) δ 8.40- 8.27, 8.12-8.01, 7.98-7.85, 7.84- 7.48 (all m, com- bined 10H), 7.38- 7.21 (m, 1H), |
| | | | | | | 3.96-3.37 (m, |
| | | | | | | 8H), 2.69, 1.98 |
| | | | | | | (each s, com- |
| | | | | | | bined 3H). MS |
| | | | | | | 427.42 (M + H). |
| 67 | 1-acetyl-2-(4- (morpholine-4- carbonyl)benz- ylidene)indolin- 3-one | 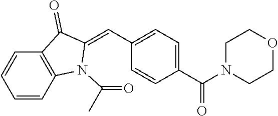 [SMILES: CC(=O)N1/C(=C\c2ccc(C(=O)N3CCOCC3)cc2)C(=O)c2ccccc21] | Z/E Mix. (about 1:1) | Ex. 60 | CAS No. 16800-68-3 (Reagent Supplier 3) CAS No. 58287-80-2 (Reagent | 1H NMR (270 MHz) δ 8.26, 8.05 (each d, each J = 8.3 Hz, combined 1H), 7.93-7.56 (m, 4H), 7.53-7.42 |
| | | | | | Supplier 6) | (m, 2H), 7.37- |
| | | | | | | 7.22 (m, 2H), |
| | | | | | | 3.93-3.31 (m, |
| | | | | | | 8H), 2.67, 2.02 |
| | | | | | | (each s, com- |
| | | | | | | bined 3H). MS |
| | | | | | | 377.37 (M + H). |
| 68 | (E)-1-acetyl-2- ((5-(morpholine- 4-carbonyl)thiazol- 2-yl)methylene)- indolin-3-one | 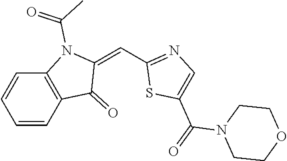 [SMILES: CC(=O)N1/C(=C/c2ncc(C(=O)N3CCOCC3)s2)C(=O)c2ccccc21] | E | Ex. 60 | Production Ex. 127 CAS No. 16800-68-3 (Reagent Supplier 3) | 1H NMR (270 MHz) δ 8.34 (s, 1H), 8.19 (s, 1H), 8.02 (d, J = 8.5 Hz, 1H), 7.92 (dd, J = 7.6, 1.5 Hz, 1H), 7.75- 7.67 (m, 1H), 7.34 (t, J = 7.5 Hz, 1H), 3.82- |
| | | | | | | 3.71 (m, 8H), |
| | | | | | | 2.74 (s, 3H). MS |
| | | | | | | 384.32 (M + H). |
| 69 | 1-acetyl-2-((1- methyl-6-(morph- oline-4-carbonyl)- 1H-indol-3-yl)- methylene)indolin- 3-one | 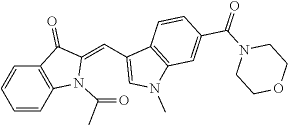 [SMILES: CC(=O)N1/C(=C\c2cn(C)c3cc(C(=O)N4CCOCC4)ccc23)C(=O)c2ccccc21] | Z/E Mix. (about 1:4) | Ex. 60 | Production Ex. 43 CAS No. 16800-68-3 (Reagent Supplier 3) | 1H NMR (500 MHz) δ 9.40 (s, 1H), 8.36-8.25, 8.03-8.00, 7.90- 7.81, 7.70-7.50 (all m, combined 6H), 7.34-7.27 (m, 2H), 3.96, |
| | | | | | | 3.90 (each s, |
| | | | | | | combined 3H), |
| | | | | | | 3.85-3.54 (m, |
| | | | | | | 8H), 2.73, 2.18 |
| | | | | | | (each s, com- |
| | | | | | | bined 3H). MS |
| | | | | | | 430.38 (M + H). |
| 70 | (Z)-1-acetyl-2- ((5-(morpholine- 4-carbonyl)pyridin- 2-yl)methylene)- indolin-3-one | 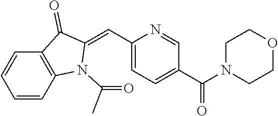 [SMILES: CC(=O)N1/C(=C\c2ccc(C(=O)N3CCOCC3)cn2)C(=O)c2ccccc21] | Z | Ex. 60 | Production Ex. 186 CAS No. 16800-68-3 (Reagent Supplier 3) | 1H NMR (270 MHz) δ 8.72 (dd, J = 4.8, 1.8 Hz, 1H), 8.04 (d, J = 8.3 Hz, 1H), 7.84 (dd, J = 7.6, 1.5 Hz, 1H), 7.72- |
| | | | | | | 7.60 (m, 2H), |
| | | | | | | 7.34-7.20 (m, |
| | | | | | | 3H), 3.98-3.74 |
| | | | | | | (m, 4H), 3.65- |
| | | | | | | 3.54 (m, 2H), |
| | | | | | | 3.34-3.24 (m, |
| | | | | | | 2H), 2.18 (s, |
| | | | | | | 3H). MS 378.57 |
| | | | | | | (M + H). |
| 71 | (Z)-1-acetyl-2- ((6-((methyl(tetra- hydro-2H-pyran- 4-yl)amino)meth- yl)quinolin-2-yl)- methylene)indolin- 3-one | 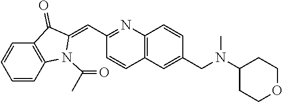 [SMILES: CC(=O)N1/C(=C\c2ccc3cc(CN(C)C4CCOCC4)ccc3n2)C(=O)c2ccccc21] | Z | Ex. 60 | Production Ex. 134 CAS No. 16800-68-3 (Reagent Supplier 3) | 1H NMR (270 MHz) δ 8.21 (d, J = 8.3 Hz, 1H), 8.16 (d, J = 8.4 Hz, 1H), 8.06 (d, J = 8.6 Hz, 1H), 7.84 (dd, J = 7.5, |
| | | | | | | 1.4 Hz, 1H), |
| | | | | | | 7.78-7.71 (m, |
| | | | | | | 2H), 7.69-7.61 |
| | | | | | | (m, 1H), 7.59 (d, |
| | | | | | | J = 8.4 Hz, 1H), |
| | | | | | | 7.35 (s, 1H), 7.24 |
| | | | | | | (t, J = 7.5 Hz, |
| | | | | | | 1H), 4.11-4.02 |
| | | | | | | (m, 2H), 3.77 (s, |
| | | | | | | 2H), 3.45-3.33 |
| | | | | | | (m, 2H), 2.78- |
| | | | | | | 2.64 (m, 1H), |
| | | | | | | 2.26 (s, 3H), 2.13 |
| | | | | | | (s, 3H), 1.87-1.63 |
| | | | | | | (m, 4H). MS |
| | | | | | | 442.31 (M + H). |
| 72 | (Z)-1-acetyl-2- ((2-(morpholine- 4-carbonyl)furo [3,2-b]pyridin-5- yl)methylene)- indolin-3-one | 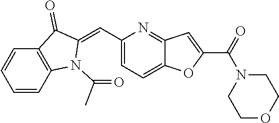 [SMILES: CC(=O)N1/C(=C\c2ccc3oc(C(=O)N4CCOCC4)cc3n2)C(=O)c2ccccc21] | Z | Ex. 60 | Production Ex. 191 CAS No. 16800-68-3 (Reagent Supplier 3) | 1H NMR (270 MHz) δ 8.25- 8.14 (m, 1H), 7.90-7.81 (m, 2H), 7.70-7.62 (m, 1H), 7.59 (d, J = 8.6 Hz, 1H), 7.41 (d, J = 1.0 |
| | | | | | | Hz, 1H), 7.33 (s, |
| | | | | | | 1H), 7.30-7.22 |
| | | | | | | (m, 1H), 3.92- |
| | | | | | | 3.75 (m, 8H), |
| | | | | | | 2.10 (s, 3H). MS |
| | | | | | | 418.32 (M + H). |
| 73 | (Z)-1-acetyl-2- ((6-(4-(oxetan-3- yl)piperazin-1- yl)quinolin-2-yl)- methylene)indolin- 3-one | 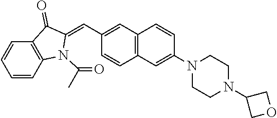 [SMILES: CC(=O)N1/C(=C\c2ccc3cc(N4CCN(C5COC5)CC4)ccc3c2)C(=O)c2ccccc21] | Z | Ex. 60 | Production Ex. 138 CAS No. 16800-68-3 (Reagent Supplier 3) | 1H NMR (270 MHz) δ 8.22 (d, J = 8.3 Hz, 1H), 8.02-7.94 (m, 2H), 7.83 (dd, J = 7.5, 1.3 Hz, 1H), 7.69-7.59 (m, 1H), 7.54- |
| | | | | | | 7.45 (m, 2H), |
| | | | | | | 7.33 (s, 1H), 7.23 |
| | | | | | | (t, J = 7.5 Hz, |
| | | | | | | 1H), 6.98 (d, J = |
| | | | | | | 2.7 Hz, 1H), |
| | | | | | | 4.77-4.64 (m, |
| | | | | | | 4H), 3.64-3.52 |
| | | | | | | (m, 1H), 3.46- |
| | | | | | | 3.38 (m, 4H), |
| | | | | | | 2.61-2.50 (m, |
| | | | | | | 4H), 2.12 (s, 3H). |
| | | | | | | MS 455.45 |
| | | | | | | (M + H). |
| 74 | (Z)-1-acetyl-2- ((6-((4-(oxetan- 3-yl)piperazin-1- yl)methyl)quinolin- 2-yl)methylene)- indolin-3-one | 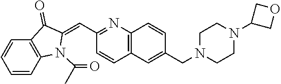 [SMILES: CC(=O)N1/C(=C\c2ccc3cc(CN4CCN(C5COC5)CC4)ccc3n2)C(=O)c2ccccc21] | Z | Ex. 60 | Production Ex. 140 CAS No. 16800-68-3 (Reagent Supplier 3) | 1H NMR (270 MHz) δ 8.21 (d, J = 8.3 Hz, 1H), 8.16 (d, J = 8.5 Hz, 1H), 8.06 (d, J = 8.7 Hz, 1H), |
| | | | | | | 7.84 (dd, J = 7.5, |
| | | | | | | 1.4 Hz, 1H), |
| | | | | | | 7.79-7.70 (m, |
| | | | | | | 2H), 7.69-7.61 |
| | | | | | | (m, 1H), 7.59 (d, |
| | | | | | | J = 8.5 Hz, 1H), |
| | | | | | | 7.34 (s, 1H), |
| | | | | | | 7.28-7.20 (m, |
| | | | | | | 1H), 4.70-4.58 |
| | | | | | | (m, 4H), 3.71 (s, |
| | | | | | | 2H), 3.59-3.46 |
| | | | | | | (m, 1H), 2.67- |
| | | | | | | 2.30 (m, 8H), |
| | | | | | | 2.13 (s, 3H). MS |
| | | | | | | 469.52 (M + H). |
| 75 | (Z)-N-((2-((1- acetyl-3-oxoin- dolin-2-ylidene)- methyl)quinolin- 6-yl)methyl)-N- (tetrahydro-2H- pyran-4-yl)acet- | 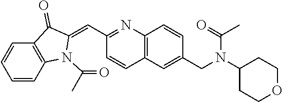 [SMILES: CC(=O)N1/C(=C\c2ccc3cc(CN(C(C)=O)C4CCOCC4)ccc3n2)C(=O)c2ccccc21] | Z | Ex. 60 | Production Ex. 146 CAS No. 16800-68-3 (Reagent Supplier 3) | 1H NMR (270 MHz) δ 8.23-8.00 (m, 3H), 7.84 (dd, J = 7.6, 1.6 Hz, 1H), 7.70-7.54 (m, 4H), 7.34 (s, 1H), 7.29-7.17 |
| | amide | | | | | (m, 1H), 4.98- |
| | | | | | | 4.82 (m, 1H), |
| | | | | | | 4.78, 4.71 (each |
| | | | | | | s, combined 2H), |
| | | | | | | 4.04-3.88 (m, |
| | | | | | | 2H), 3.57-3.33 |
| | | | | | | (m, 2H), 2.35, |
| | | | | | | 2.15 (each s, |
| | | | | | | combined 3H), |
| | | | | | | 2.12 (s, 3H), |
| | | | | | | 1.93-1.56 (m, |
| | | | | | | 4H). MS 470.38 |
| | | | | | | (M + H). |
| 76 | (Z)-N-((2-((1- acetyl-3-oxoin- dolin-2-ylidene)- methyl)quinolin- 6-yl)methyl)-N- (tetrahydro-2H- pyran-4-yl)meth- | 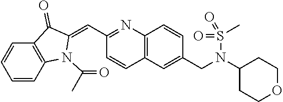 [SMILES: CC(=O)N1/C(=C\c2ccc3cc(CN(C4CCOCC4)S(C)(=O)=O)ccc3n2)C(=O)c2ccccc21] | Z | Ex. 60 | Production Ex. 147 CAS No. 16800-68-3 (Reagent Supplier 3) | 1H NMR (270 MHz) δ 8.23-8.16 (m, 2H), 8.09 (d, J = 8.8 Hz, 1H), 7.87-7.81 (m, 2H), 7.77 (dd, J = 8.7, 2.0 Hz, 1H), |
| | anesulfonamide | | | | | 7.70-7.58 (m, |
| | | | | | | 2H), 7.34 (s, 1H), |
| | | | | | | 7.29-7.20 (m, 1H), |
| | | | | | | 4.60 (s, 2H), |
| | | | | | | 4.14-3.89 (m, |
| | | | | | | 3H), 3.48-3.32 |
| | | | | | | (m, 2H), 2.91 |
| | | | | | | (s, 3H), 2.14 (s, |
| | | | | | | 3H), 1.91-1.60 |
| | | | | | | (m, 4H). MS |
| | | | | | | 506.35 (M + H). |
| 77 | (Z)-1-acetyl-2- ((6-((E)-3-morph- olino-3-oxoprop- 1-en-1-yl)quino- lin-2-yl)methyl- ene)indolin-3-one | 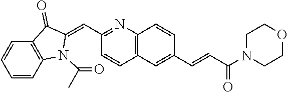 [SMILES: CC(=O)N1/C(=C\c2ccc3cc(/C=C/C(=O)N4CCOCC4)ccc3n2)C(=O)c2ccccc21] | Z | Ex. 60 | Production Ex. 149 CAS No. 16800-68-3 (Reagent Supplier 3) | 1H NMR (500 MHz) δ 8.23-8.17 (m, 2H), 8.08 (d, J = 8.8 Hz, 1H), 7.94 (dd, J = 8.9, 2.0 Hz, 1H), |
| | | | | | | 7.89-7.82 (m, |
| | | | | | | 3H), 7.69-7.63 |
| | | | | | | (m, 1H), 7.62 (d, |
| | | | | | | J = 8.5 Hz, 1H), |
| | | | | | | 7.33 (s, 1H), |
| | | | | | | 7.28-7.23 (m, |
| | | | | | | 1H), 7.01 (d, J = |
| | | | | | | 15.4 Hz, 1H), |
| | | | | | | 3.82-3.69 (m, |
| | | | | | | 8H), 2.15 (s, 3H). |
| | | | | | | MS 454.40 |
| | | | | | | (M + H). |
| 78 | (Z)-1-acetyl-2- ((6-((E)-3-(4- (oxetan-3-yl)- piperazin-1-yl)- 3-oxoprop-1-en- | 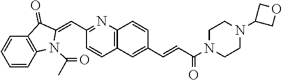 [SMILES: CC(=O)N1/C(=C\c2ccc3cc(/C=C/C(=O)N4CCN(C5COC5)CC4)ccc3n2)C(=O)c2ccccc21] | Z | Ex. 60 | Production Ex. 183 CAS No. 16800-68-3 (Reagent | 1H NMR (500 MHz) δ 8.22-8.18 (m, 2H), 8.08 (d, J = 8.8 Hz, 1H), 7.94 (dd, J = 9.1, |
| | 1-yl)quinolin-2- | | | | Supplier 3) | 1.9 Hz, 1H), |
| | yl)methylene)- | | | | | 7.88-7.79 (m, |
| | indolin-3-one | | | | | 3H), 7.69-7.64 |
| | | | | | | (m, 1H), 7.61 (d, |
| | | | | | | J = 8.4 Hz, 1H), |
| | | | | | | 7.33 (s, 1H), |
| | | | | | | 7.27-7.22 (m, |
| | | | | | | 1H), 7.03 (d, J = |
| | | | | | | 15.4 Hz, 1H), |
| | | | | | | 4.73-4.62 (m, |
| | | | | | | 4H), 3.87-3.71 |
| | | | | | | (m, 4H), 3.57- |
| | | | | | | 3.49 (m, 1H), |
| | | | | | | 2.46-2.34 (m, |
| | | | | | | 4H), 2.14 (s, |
| | | | | | | 3H). MS 509.48 |
| | | | | | | (M + H). |
| 79 | (E)-3-(2-(((Z)-1- acetyl-3-oxoin- dolin-2-ylidene)- methyl)quinolin- 6-yl)-N-(1- (oxetan-3-yl)- | 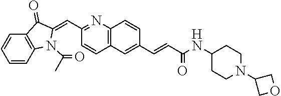 [SMILES: CC(=O)N1/C(=C\c2ccc3cc(/C=C/C(=O)NC4CCN(C5COC5)CC4)ccc3n2)C(=O)c2ccccc21] | Z | Ex. 60 | Production Ex. 184 CAS No. 16800-68-3 (Reagent Supplier 3) | 1H NMR (500 MHz) δ 8.22-8.16 (m, 2H), 8.06 (d, J = 8.9 Hz, 1H), 7.90 (dd, J = 8.8, 1.9 Hz, 1H), |
| | piperidin-4-yl)- | | | | | 7.86-7.82 (m, |
| | acrylamide | | | | | 2H), 7.79 (d, J = |
| | | | | | | 15.6 Hz, 1H), |
| | | | | | | 7.68-7.63 (m, |
| | | | | | | 1H), 7.60 (d, J = |
| | | | | | | 8.4 Hz, 1H), 7.32 |
| | | | | | | (s, 1H), 7.27-7.22 |
| | | | | | | (m, 1H), 6.55 (d, |
| | | | | | | J = 15.5 Hz, 1H), |
| | | | | | | 5.71 (d, J = 8.1 |
| | | | | | | Hz, 1H), 4.70- |
| | | | | | | 4.59 (m, 4H), |
| | | | | | | 4.05-3.95 (m, |
| | | | | | | 1H), 3.55-3.46 |
| | | | | | | (m, 1H), 2.78- |
| | | | | | | 2.71 (m, 2H), |
| | | | | | | 2.14 (s, 3H), |
| | | | | | | 2.10-1.99 (m, |
| | | | | | | 4H), 1.65-1.54 |
| | | | | | | (m, 2H). MS |
| | | | | | | 523.46 (M + H). |
| 80 | (Z)-N-((2-((1- acetyl-3-oxoin- dolin-2-ylidene)- methyl)quinolin- 6-yl)methyl)-2- morpholinoacet- | 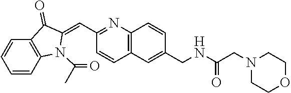 [SMILES: CC(=O)N1/C(=C\c2ccc3cc(CNC(=O)CN4CCOCC4)ccc3n2)C(=O)c2ccccc21] | Z | Ex. 60 | Production Ex. 150 CAS No. 16800-68-3 (Reagent Supplier 3) | 1H NMR (270 MHz) δ 8.25-8.12 (m, 2H), 8.09 (d, J = 8.6 Hz, 1H), 7.84 (dd, J = 7.5, 1.4 Hz, 1H), |
| | amide | | | | | 7.72-7.57 (m, |
| | | | | | | 4H), 7.34 (s, 1H), |
| | | | | | | 7.29-7.20 (m, |
| | | | | | | 1H), 4.68 (d, J = |
| | | | | | | 6.3 Hz, 2H), |
| | | | | | | 3.74-3.67 (m, |
| | | | | | | 4H), 3.13 (s, 2H), |
| | | | | | | 2.60-2.54 (m, |
| | | | | | | 4H), 2.12 (s, 3H). |
| | | | | | | MS 471.38 |
| | | | | | | (M + H). |
| 81 | (Z)-1-acetyl-2- ((4-azido-6- (morpholine- 4-carbonyl)quin- olin-2-yl)methyl- ene)indolin-3-one | 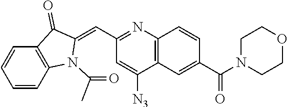 [SMILES: CC(=O)N1/C(=C\c2cc(N=[N+]=[N-])c3cc(C(=O)N4CCOCC4)ccc3n2)C(=O)c2ccccc21] | Z | Ex. 60 | Production Ex. 151 CAS No. 16800-68-3 (Reagent Supplier 3) | 1H NMR (270 MHz) δ 8.16 (d, J = 8.2 Hz, 1H), 8.13-8.02 (m, 2H), 7.85 (dd, J = 7.5, 1.5 Hz, 1H), 7.78 (dd, J = 8.6, |
| | | | | | | 1.9 Hz, 1H), 7.71- |
| | | | | | | 7.62 (m, 1H), |
| | | | | | | 7.36 (s, 1H), |
| | | | | | | 7.30-7.23 (m, |
| | | | | | | 2H), 3.97-3.34 |
| | | | | | | (m, 8H), 2.14 (s, |
| | | | | | | 3H). MS 469.28 |
| | | | | | | (M + H). |
| 82 | (Z)-1-acetyl-2- ((4-chloro-6- (morpholine-4- carbonyl)quin- olin-2-yl)methyl- ene)indolin-3-one | 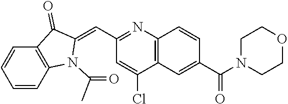 [SMILES: CC(=O)N1/C(=C\c2cc(Cl)c3cc(C(=O)N4CCOCC4)ccc3n2)C(=O)c2ccccc21] | Z | Ex. 60 | Production Ex. 153 CAS No. 16800-68-3 (Reagent Supplier 3) | 1H NMR (270 MHz) δ 8.29 (d, J = 1.7 Hz, 1H), 8.15 (d, J = 8.3 Hz, 2H), 7.85 (dd, J = 7.5, 1.4 Hz, 1H), 7.82- |
| | | | | | | 7.77 (m, 1H), |
| | | | | | | 7.72 (s, 1H), |
| | | | | | | 7.71-7.63 (m, |
| | | | | | | 1H), 7.33-7.25 |
| | | | | | | (m, 1H), 7.23 |
| | | | | | | (s, 1H), 3.99- |
| | | | | | | 3.39 (m, 8H), |
| | | | | | | 2.15 (s, 3H). MS |
| | | | | | | 462.26 (M + H). |
| 83 | (Z)-1-acetyl-2- ((6-(morpholine- 4-carbonyl)-4- phenylquinolin-2- yl)methylene)in- dolin-3-one | 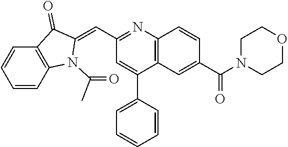 [SMILES: CC(=O)N1/C(=C\c2cc(-c3ccccc3)c3cc(C(=O)N4CCOCC4)ccc3n2)C(=O)c2ccccc21] | Z | Ex. 60 | Production Ex. 154 CAS No. 16800-68-3 (Reagent Supplier 3) | 1H NMR (270 MHz) δ 8.20 (d, J = 8.6 Hz, 2H), 7.97 (d, J = 1.8 Hz, 1H), 7.85 (dd, J = 7.5, 1.4 Hz, 1H), 7.75 (dd, J = 8.6, 1.9 Hz, 1H), 7.71-7.63 (m, |
| | | | | | | 1H), 7.60-7.48 |
| | | | | | | (m, 6H), 7.35 (s, |
| | | | | | | 1H), 7.26 (t, J = |
| | | | | | | 7.3 Hz, 1H), 3.92- |
| | | | | | | 3.32 (m, 8H), |
| | | | | | | 2.20 (s, 3H). MS |
| | | | | | | 504.35 (M + H). |
| 84 | (Z)-1-acetyl-2- ((4-butoxy-6- (morpholine-4- carbonyl)quinolin- 2-yl)methylene)- indolin-3-one | 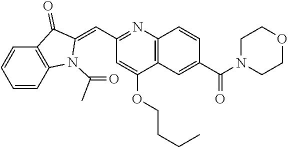 [SMILES: CCCCOc1cc(/C=C2/C(=O)c3ccccc3N2C(C)=O)nc2ccc(C(=O)N3CCOCC3)cc12] | Z | Ex. 60 | Production Ex. 155 CAS No. 16800-68-3 (Reagent Supplier 3) | 1H NMR (270 MHz) δ 8.28 (d, J = 1.8 Hz, 1H), 8.19 (d, J = 8.3 Hz, 1H), 8.04 (d, J = 8.6 Hz, 1H), 7.84 (dd, J = 7.6, 1.4 Hz, 1H), 7.74-7.61 (m, |
| | | | | | | 2H), 7.29-7.20 |
| | | | | | | (m, 2H), 6.97 (s, |
| | | | | | | 1H), 4.24 (t, J = |
| | | | | | | 6.4 Hz, 2H), |
| | | | | | | 4.00-3.38 (m, |
| | | | | | | 8H), 2.10 (s, 3H), |
| | | | | | | 2.02-1.88 (m, |
| | | | | | | 2H), 1.69-1.52 |
| | | | | | | (m, 2H), 1.05 (t, |
| | | | | | | J = 7.3 Hz, 3H). |
| | | | | | | MS 500.33 |
| | | | | | | (M + H). |
| 85 | (Z)-1-acetyl-2- ((6-(morpholine- 4-carbonyl)-4- (pyridin-3-yl)quin- olin-2-yl)methyl- ene)indolin-3-one | 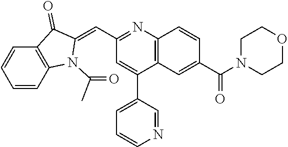 [SMILES: CC(=O)N1/C(=C\c2cc(-c3cccnc3)c3cc(C(=O)N4CCOCC4)ccc3n2)C(=O)c2ccccc21] | Z | Ex. 60 | Production Ex. 156 CAS No. 16800-68-3 (Reagent Supplier 3) | 1H NMR (270 MHz) δ 8.84- 8.75 (m, 2H), 8.23 (d, J = 8.7 Hz, 1H), 8.18 (d, J = 8.3 Hz, 1H), 7.91-7.82 (m, 3H), 7.77 (dd, J = 8.7, 1.8 Hz, 1H), |
| | | | | | | 7.72-7.64 (m, |
| | | | | | | 1H), 7.58 (s, 1H), |
| | | | | | | 7.57-7.50 (m, |
| | | | | | | 1H), 7.35 (s, 1H), |
| | | | | | | 7.31-7.22 (m, |
| | | | | | | 1H), 3.93-3.31 |
| | | | | | | (m, 8H), 2.22 (s, |
| | | | | | | 3H). MS 505.32 |
| | | | | | | (M + H). |
| 86 | (Z)-2-((1-acetyl- 3-oxoindolin-2- ylidene)methyl)- 6-(morpholine-4- carbonyl)quino- line-4-carbonitrile | 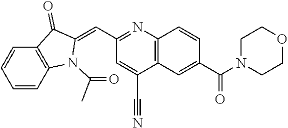 [SMILES: CC(=O)N1/C(=C\c2cc(C#N)c3cc(C(=O)N4CCOCC4)ccc3n2)C(=O)c2ccccc21] | Z | Ex. 60 | Production Ex. 157 CAS No. 16800-68-3 (Reagent Supplier 3) | 1H NMR (270 MHz) δ 8.26- 8.19 (m, 2H), 8.09 (d, J = 8.3 Hz, 1H), 7.93 (s, 1H), 7.92-7.83 (m, 2H), 7.72- 7.64 (m, 1H), |
| | | | | | | 7.32-7.25 (m, |
| | | | | | | 2H), 3.99-3.39 |
| | | | | | | (m, 8H), 2.20 (s, |
| | | | | | | 3H). MS 453.18 |
| | | | | | | (M + H). |
| 87 | 1-(2-((1-acetyl-3- oxoindolin-2-yl- idene)methyl)-6- (morpholine-4- carbonyl)quinolin- 4-yl)piperidine-4- carbonitrile | 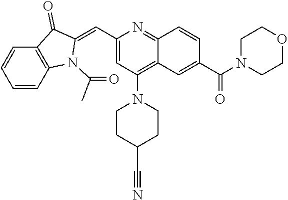 [SMILES: CC(=O)N1/C(=C\c2cc(N3CCC(C#N)CC3)c3cc(C(=O)N4CCOCC4)ccc3n2)C(=O)c2ccccc21] | Z/E Mix. (about 7:1) | Ex. 60 | Production Ex. 159 CAS No. 16800-68-3 (Reagent Supplier 3) | 1H NMR (270 MHz) δ 8.21-8.14 (m, 1H), 8.11- 7.97 (m, 2H), 7.88-7.80 (m, 1H), 7.71-7.61 (m, 2H), 7.31- 7.21 (m, 2H), 7.10 (s, 1H), 3.94-3.40 (m, 10H), 3.29-3.15 (m, 2H), 3.03- |
| | | | | | | 2.90 (m, 1H), |
| | | | | | | 2.32-2.13 (m, |
| | | | | | | 4H), 2.12 (s, 3H). |
| | | | | | | MS 536.43 |
| | | | | | | (M + H). |
| 88 | (Z)-2-((4-([1,1′- biphenyl]-3-yl)-6- (morpholine-4- carbonyl)quinolin- 2-yl)methylene)-1- acetylindolin-3-one | 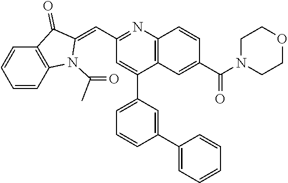 [SMILES: CC(=O)N1/C(=C\c2cc(-c3cccc(-c4ccccc4)c3)c3cc(C(=O)N4CCOCC4)ccc3n2)C(=O)c2ccccc21] | Z | Ex. 60 | Production Ex. 160 CAS No. 16800-68-3 (Reagent Supplier 3) | 1H NMR (270 MHz) δ 8.24-8.17 (m, 2H), 8.01 (d, J = 1.8 Hz, 1H), 7.85 (dd, J = 7.7, 1.3 Hz, 1H), 7.81-7.73 (m, 2H), 7.73-7.59 (m, 6H), 7.53- 7.38 (m, 4H), 7.36 (s, 1H), |
| | | | | | | 7.30-7.22 (m, |
| | | | | | | 1H), 3.93-3.28 |
| | | | | | | (m, 8H), 2.21 (s, |
| | | | | | | 3H). MS 580.40 |
| | | | | | | (M + H). |
| 89 | (Z)-2-((4-([1,1′- biphenyl]-2-yl)-6- (morpholine-4- carbonyl)quinolin- 2-yl)methylene)- 1-acetylindolin-3- one | 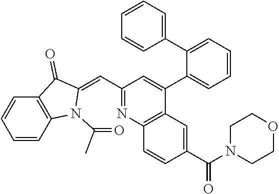 [SMILES: CC(=O)N1/C(=C\c2cc(-c3ccccc3-c3ccccc3)c3cc(C(=O)N4CCOCC4)ccc3n2)C(=O)c2ccccc21] | Z | Ex. 60 | Production Ex. 161 CAS No. 16800-68-3 (Reagent Supplier 3) | 1H NMR (270 MHz) δ 8.19 (d, J = 8.5 Hz, 1H), 8.08 (d, J = 8.4 Hz, 1H), 7.83 (dd, J = 7.6, 1.3 Hz, 1H), 7.70- 7.49 (m, 6H), 7.44-7.37 (m, 2H), 7.29-7.21 (m, 1H), 7.20 (s, 1H), 7.05-7.00 |
| | | | | | | (m, 5H), 3.92- |
| | | | | | | 3.17 (m, 8H), |
| | | | | | | 2.02 (s, 3H). MS |
| | | | | | | 580.3 (M + H). |
| 90 | (Z)-1-acetyl-2- ((4-(4-acetyl- piperazin-1-yl)- 6-(morpholine-4- carbonyl)quinolin- 2-yl)methylene)- indolin-3-one | 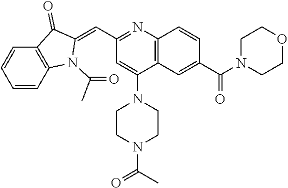 [SMILES: CC(=O)N1CCN(c2cc(/C=C3/C(=O)c4ccccc4N3C(C)=O)nc3ccc(C(=O)N4CCOCC4)cc23)CC1] | Z | Ex. 60 | Production Ex. 189 CAS No. 16800-68-3 (Reagent Supplier 3) | 1H NMR (500 MHz) δ 8.19- 8.15 (m, 1H), 8.11-8.05 (m, 2H), 7.86-7.81 (m, 1H), 7.71- 7.62 (m, 2H), 7.27-7.21 (m, 2H), 7.08 (s, 1H), 4.01-3.40 (m, 12H), 3.31-3.21 (m, 4H), 2.19 (s, |
| | | | | | | 3H), 2.12 (s, 3H). |
| | | | | | | MS 554.43 |
| | | | | | | (M + H). |
| 91 | (Z)-1-acetyl-2- ((6-(morpholine- 4-carbonyl)-[4,8′- biquinolin]-2-yl)- methylene)indolin- 3-one | 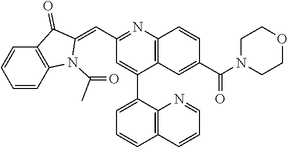 [SMILES: CC(=O)N1/C(=C\c2cc(-c3cccc4cccnc34)c3cc(C(=O)N4CCOCC4)ccc3n2)C(=O)c2ccccc21] | Z | Ex. 60 | Production Ex. 162 CAS No. 16800-68-3 (Reagent Supplier 3) | 1H NMR (500 MHz) δ 8.76 (dd, J = 4.1, 1.8 Hz, 1H), 8.32 (dd, J = 8.3, 1.8 Hz, 1H), 8.26-8.16 (m, 2H), 8.04 (dd, J = 7.7, 1.9 Hz, 1H), 7.87-7.81 (m, |
| | | | | | | 1H), 7.77-7.71 |
| | | | | | | (m, 3H), 7.71 (s, |
| | | | | | | 1H), 7.68-7.62 |
| | | | | | | (m, 1H), 7.48 |
| | | | | | | (dd, J = 8.3, 4.1 |
| | | | | | | Hz, 1H), 7.43 (d, |
| | | | | | | J = 1.8 Hz, 1H), |
| | | | | | | 7.39 (s, 1H), |
| | | | | | | 7.27-7.22 (m, |
| | | | | | | 1H), 3.79-3.16 |
| | | | | | | (m, 8H), 2.29 (s, |
| | | | | | | 3H). MS 555.33 |
| | | | | | | (M + H). |
| 92 | (Z)-1-acetyl-2-((6- (morpholine-4- carbonyl)-4-(4- phenoxyphenyl)- quinolin-2-yl)- methylene)in- dolin-3-one | 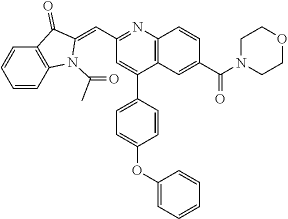 [SMILES: CC(=O)N1/C(=C\c2cc(-c3ccc(Oc4ccccc4)cc3)c3cc(C(=O)N4CCOCC4)ccc3n2)C(=O)c2ccccc21] | Z | Ex. 60 | Production Ex. 163 CAS No. 16800-68-3 (Reagent Supplier 3) | 1H NMR (270 MHz) δ 8.20 (d, J = 8.4 Hz, 2H), 8.04 (d, J = 1.9 Hz 1H), 7.85 (dd, J = 7.6, 1.4 Hz, 1H), 7.74 (dd, J = 8.7, 1.9 Hz, 1H), 7.71-7.63 (m, 1H), 7.58 (s, 1H), 7.52-7.38 (m, 5H), 7.35 (s, 1H), 7.30-7.10 (m, 5H), 3.95-3.32 |
| | | | | | | (m, 8H), 2.19 (s, |
| | | | | | | 3H). MS 596.3 |
| | | | | | | (M + H). |
| 93 | 1-acetyl-2-((1- methyl-5-(morph- oline-4-carbonyl)- 1H-indol-2-yl)- methylene)indolin- 3-one | 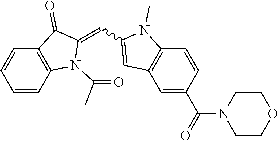 [SMILES: CC(=O)N1C(=Cc2cc3cc(C(=O)N4CCOCC4)ccc3n2C)C(=O)c2ccccc21] | Z/E Mix. (about 4:5) | Ex. 60 | Production Ex. 193 CAS No. 16800-68-3 (Reagent Supplier 3) | 1H NMR (500 MHz) δ 8.39- 8.25, 7.91-7.62, 7.41-7.28, 6.93- 6.89 (all m, com- bined 9H), 3.91, 3.86 (each s,com- bined 3H), 3.83- 3.55 (m, 8H), |
| | | | | | | 2.72, 2.16 (each |
| | | | | | | s, combined 3H). |
| | | | | | | MS 430.37 |
| | | | | | | (M + H). |
| 94 | 1-acetyl-2-((6- (morpholine-4- carbonyl)-4-(2- phenylpyridin-3- yl)quinolin-2-yl)- methylene)indolin- 3-one | 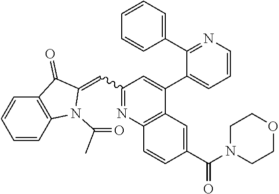 [SMILES: CC(=O)N1C(=Cc2cc(-c3cccnc3-c3ccccc3)c3cc(C(=O)N4CCOCC4)ccc3n2)C(=O)c2ccccc21] | Z/E Mix. (about 7:1) | Ex. 60 | Production Ex. 176 CAS No. 16800-68-3 (Reagent Supplier 3) | 1H NMR (500 MHz) δ 8.94-8.86 (m, 1H), 8.19- 8.03 (m, 2H), 7.93-7.61 (m, 4H), 7.58-7.40 (m, 3H), 7.34- 7.20 (m, 4H), 7.15-7.01 (m, 3H), 3.95-3.00 (m, 8H), 2.75, 2.10 (each s, |
| | | | | | | combined 2H). |
| | | | | | | MS 581.34 |
| | | | | | | (M + H) |
| 95 | (Z)-2-((4-([1,1′- biphenyl]-2-yl)-6- (4-(oxetan-3-yl)- piperazine-1- carbonyl)quinolin- 2-yl)methylene)- 1-acetylindolin-3- one | 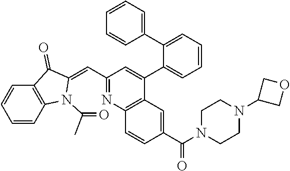 [SMILES: CC(=O)N1/C(=C\c2cc(-c3ccccc3-c3ccccc3)c3cc(C(=O)N4CCN(C5COC5)CC4)ccc3n2)C(=O)c2ccccc21] | Z | Ex. 60 | Production Ex. 178 CAS No. 16800-68-3 (Reagent Supplier 3) | 1H NMR (500 MHz) δ 8.18-8.15 (m, 1H), 8.05 (dd, J = 8.6, 0.6 Hz, 1H), 7.82-7.79 (m, 1H), 7.66- 7.54 (m, 5H), 7.52-7.48 (m, 1H), 7.40-7.37 (m, 1H), 7.36 (s, 1H), 7.24-7.20 |
| | | | | | | (m, 1H), 7.17 |
| | | | | | | (s, 1H), 7.04- |
| | | | | | | 6.97 (m, 5H), |
| | | | | | | 4.71-4.64 (m, |
| | | | | | | 2H), 4.60 (t, J = |
| | | | | | | 6.2 Hz, 2H), |
| | | | | | | 3.96-3.62 (m, |
| | | | | | | 2H), 3.57-3.46 |
| | | | | | | (m, 1H), 3.41- |
| | | | | | | 3.18 (m, 2H), |
| | | | | | | 2.52-2.08 (m, |
| | | | | | | 4H), 2.00 (s, |
| | | | | | | 3H). MS 635.44 |
| | | | | | | (M + H) |
| 96 | (Z)-2-((4-([1,1′- biphenyl]-2-yl)-6- (4-(2-hydroxy-3- methoxypropyl)- piperazine-1-car- bonyl)quinolin-2- yl)methylene)-1- acetylindolin-3-one | 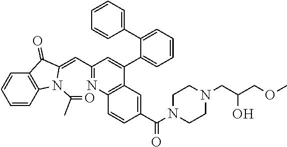 [SMILES: COCC(O)CN1CCN(C(=O)c2ccc3nc(/C=C4/C(=O)c5ccccc5N4C(C)=O)cc(-c4ccccc4-c4ccccc4)c3c2)CC1] | Z | Ex. 60 | Production Ex. 179 CAS No. 16800-68-3 (Reagent Supplier 3) | 1H NMR (500 MHz) δ 8.18-8.15 (m, 1H), 8.07- 8.03 (m, 1H), 7.82-7.78 (m, 1H), 7.65-7.53 (m, 5H), 7.52- 7.48 (m, 1H), 7.40-7.35 (m, |
| | | | | | | 2H), 7.24-7.20 |
| | | | | | | (m, 1H), 7.17 (s, |
| | | | | | | 1H), 7.04-6.97 |
| | | | | | | (m, 5H), 3.96- |
| | | | | | | 3.04 (m, 10H, |
| | | | | | | 2.80-2.25 (m, |
| | | | | | | 6H), 1.99 (s, 3H). |
| | | | | | | MS 667.44 |
| | | | | | | (M + H) |
| 97 | (Z)-4-([1,1′- biphen-yl]-2-yl)- 2-((1-acetyl-3- oxoindolin-2-yl- idene)methyl)-N- (1-(oxetan-3-yl)- piperidin-4-yl)- quinoline-6- carboxamide | 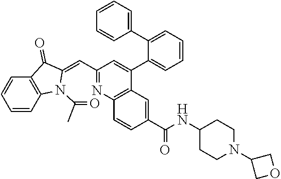 [SMILES: CC(=O)N1/C(=C\c2cc(-c3ccccc3-c3ccccc3)c3cc(C(=O)NC4CCN(C5COC5)CC4)ccc3n2)C(=O)c2ccccc21] | Z | Ex. 60 | Production Ex. 180 CAS No. 16800-68-3 (Reagent Supplier 3) | 1H NMR (500 MHz) δ 8.19-8.15 (m, 1H), 8.08- 8.04 (m, 1H), 7.94-7.89 (m, 2H), 7.85-7.79 (m, 1H), 7.67- 7.58 (m, 3H), 7.57-7.53 (m, 1H), 7.47-7.44 (m, 2H), 7.26- |
| | | | | | | 7.20 (m, 2H), |
| | | | | | | 7.09-6.99 (m, |
| | | | | | | 5H), 5.96 (d, J = |
| | | | | | | 7.9 Hz, 1H), 4.68 |
| | | | | | | (t, J = 6.6 Hz, |
| | | | | | | 2H), 4.63-4.59 |
| | | | | | | (m, 2H), 4.03- |
| | | | | | | 3.94 (m, 1H), |
| | | | | | | 3.53-3.46 (m, |
| | | | | | | 1H), 2.80-2.72 |
| | | | | | | (m, 2H), 2.13- |
| | | | | | | 1.97 (m, 4H), |
| | | | | | | 1.66-1.55 (m, |
| | | | | | | 2H). MS 649.48 |
| 98 | (Z)-1-acetyl-2- ((5-(2,6-dimethyl- morpholine-4- carbonyl)benzo [d]thiazol-2-yl)- methylene)indolin- 3-one | 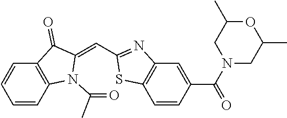 [SMILES: CC(=O)N1/C(=C\c2nc3cc(C(=O)N4CC(C)OC(C)C4)ccc3s2)C(=O)c2ccccc21] | Z | | Production Ex. 142 CAS No. 16800-68-3 (Reagent Supplier 3) | 1H NMR (270 MHz) δ 8.18 (d, J = 8.3 Hz, 1H), 8.12 (d, J = 1.7 Hz, 1H), 7.99 (d, J = 8.3 Hz, 1H), 7.85 (dd, J = 7.5, 1.4 Hz, 1H), |
| | | | | | | 7.73-7.64 (m, |
| | | | | | | 1H), 7.52 (dd, J = |
| | | | | | | 8.3, 1.6 Hz, 1H), |
| | | | | | | 7.32 (s, 1H), |
| | | | | | | 7.31-7.26 (m, |
| | | | | | | 1H), 4.73-4.49 |
| | | | | | | (m, 2H), 3.78- |
| | | | | | | 3.44 (m, 4H), |
| | | | | | | 2.26 (s, 3H), |
| | | | | | | 1.35-1.01 (m, |
| | | | | | | 6H). MS 462.39 |
| | | | | | | (M + H). |
| 99 | (E)-1-acetyl-2-((5- (2,6-dimethylmor- pholine-4-carbon- yl)benzo[d]thiazol- 2-yl)methylene)- indolin-3-one | 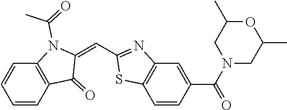 [SMILES: CC(=O)N1/C(=C/c2nc3cc(C(=O)N4CC(C)OC(C)C4)ccc3s2)C(=O)c2ccccc21] | E | | Production Ex. 142 CAS No. 16800-68-3 (Reagent Supplier 3) | 1H NMR (270 MHz) δ 8.33 (s, 1H), 8.12-8.00 (m, 3H), 7.92 (dd, J = 7.8, 1.5 Hz, 1H), 7.76- 7.67 (m, 1H), |
| | | | | | | 7.54 (dd, J = 8.3, |
| | | | | | | 1.6 Hz, 1H), |
| | | | | | | 7.38-7.29 (m, |
| | | | | | | 1H), 4.74-4.47 |
| | | | | | | (m, 2H), 3.81- |
| | | | | | | 3.47 (m, 4H), |
| | | | | | | 2.76 (s, 3H), |
| | | | | | | 1.39-0.99 (m, |
| | | | | | | 6H). MS 462.41 |
| | | | | | | (M + H). |
| 100 | (Z)-1-acetyl-2- ((6-(2,6-dimethyl- morpholine-4- carbonyl)benzo [d]thiazol-2-yl)- methylene)indolin- 3-one | 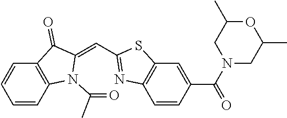 [SMILES: CC(=O)N1/C(=C\c2nc3ccc(C(=O)N4CC(C)OC(C)C4)cc3s2)C(=O)c2ccccc21] | Z | Ex. 98 | Production Ex. 143 CAS No. 16800-68-3 (Reagent Supplier 3) | 1H NMR (270 MHz) δ 8.18 (d, J = 8.3 Hz, 1H), 8.13 (d, J = 8.3 Hz, 1H), 8.01 (d, J = 1.6 Hz, 1H), 7.85 (dd, J = 7.5, 1.4 Hz, 1H), |
| | | | | | | 7.72-7.64 (m, |
| | | | | | | 1H), 7.55 (dd, |
| | | | | | | J = 8.5, 1.7 Hz, |
| | | | | | | 1H), 7.33 (s, 1H), |
| | | | | | | 7.31-7.25 (m, |
| | | | | | | 1H), 4.72-4.44 |
| | | | | | | (m, 2H), 3.77- |
| | | | | | | 3.44 (m, 4H), |
| | | | | | | 2.26 (s, 3H), |
| | | | | | | 1.37-1.02 (m, |
| | | | | | | 6H). MS 462.40 |
| | | | | | | (M + H). |
| 101 | (E)-1-acetyl-2- ((6-(2,6-dimethyl- morpholine-4- carbonyl)benzo [d]thiazol-2-yl)- methylene)indolin- 3-one | 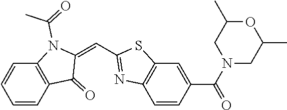 [SMILES: CC(=O)N1/C(=C/c2nc3ccc(C(=O)N4CC(C)OC(C)C4)cc3s2)C(=O)c2ccccc21] | E | Ex. 99 | Production Ex. 143 CAS No. 16800-68-3 (Reagent Supplier 3) | 1H NMR (270 MHz) δ 8.33 (s, 1H), 8.12 (d, J = 8.3 Hz, 1H), 8.09- 8.03 (m, 2H), 7.92 (dd, J = 7.8, 1.3 Hz, 1H), 7.76- |
| | | | | | | 7.68 (m, 1H), |
| | | | | | | 7.55 (dd, J = 8.3, |
| | | | | | | 1.6 Hz, 1H), 7.35 |
| | | | | | | (t, J = 7.6 Hz, |
| | | | | | | 1H), 4.72-4.47 |
| | | | | | | (m, 2H), 3.78- |
| | | | | | | 3.46 (m, 4H), |
| | | | | | | 2.76 (s, 3H), |
| | | | | | | 1.37-1.03 (m, |
| | | | | | | 6H). MS 462.42 |
| | | | | | | (M + H). |
| 102 | 1-acetyl-2-((5-(2- morpholino-2-oxo- ethoxy)pyridin-2- yl)methylene)-1,2- dihydro-3H-pyr- rolo[2,3-b]pyridin- 3-one | 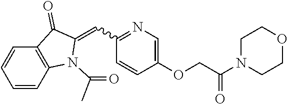 [SMILES: CC(=O)N1C(=Cc2ccc(OCC(=O)N3CCOCC3)cn2)C(=O)c2ccccc21] | Z/E Mix (about 4:1) | | CAS No. 1511998- 57-4 CAS No. 155818- 89-6 (Reagent Supplier 1 | 1H NMR (270 MHz) δ 8.64-8.55 (m, 1H), 8.43- 8.39 (m, 1H), 8.21-8.03 (m, 1H), 7.47-7.25 (m, 3H), 7.23- 7.16 (m, 1H), |
| | | | | | for each | 4.80, 4.78 (each |
| | | | | | of them) | s, combined 2H), |
| | | | | | | 3.76-3.56 (m, |
| | | | | | | 8H), 2.96, 2.90 |
| | | | | | | (each s, com- |
| | | | | | | bined 3H). MS |
| | | | | | | 409.41 (M + H). |
| 103 | 1-acetyl-6-fluoro- 2-(3-methoxy-4- (2-morpholino-2- oxoethoxy)benz- ylidene)indolin-3- one | 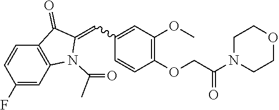 [SMILES: COc1cc(C=C2C(=O)c3ccc(F)cc3N2C(C)=O)ccc1OCC(=O)N1CCOCC1] | Z/E Mix. (about 4:1) | | CAS No. 68438-39-1 (Reagent Supplier 1) CAS No. 31438-76-3 (Reagent | 1H NMR (270 MHz) δ 8.12- 7.75 (m, 2H), 7.34-7.25 (m, 2H), 7.17-6.92 (m, 3H), 4.83, 4.82 (each s, |
| | | | | | Supplier 9) | combined 2H), |
| | | | | | | 4.01, 3.88 (each |
| | | | | | | s, combined |
| | | | | | | 3H), 3.72-3.58 |
| | | | | | | (m, 8H), 2.62, |
| | | | | | | 2.03 (each s, |
| | | | | | | combined 3H). |
| | | | | | | MS 455.33 |
| | | | | | | (M + H). |
| 104 | 1-acetyl-4-fluoro- 2-(3-methoxy-4- (2-morpholino-2- oxoethoxy)benz- ylidene)indolin-3- one | 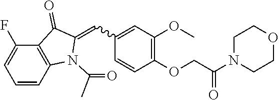 [SMILES: COc1cc(C=C2C(=O)c3c(F)cccc3N2C(C)=O)ccc1OCC(=O)N1CCOCC1] | Z/E Mix. (about 3:1) | | CAS No. 68438-40-4 (Reagent Supplier 1) CAS No. 31438-76-3 (Reagent | 1H NMR (270 MHz) δ 8.12-7.96 (m, 1H), 7.68- 7.53 (m, 1H), 7.35-7.26, 7.17- 7.06 (each m, combined 3H), |
| | | | | | Supplier 9) | 7.01-6.85 (m, |
| | | | | | | 2H), 4.83, 4.81 |
| | | | | | | (each s, com- |
| | | | | | | bined 2H), 4.01, |
| | | | | | | 3.88 (each s, |
| | | | | | | combined 3H), |
| | | | | | | 3.73-3.58 (m, |
| | | | | | | 8H), 2.62, 2.03 |
| | | | | | | (each s, com- |
| | | | | | | bined 3H). MS |
| | | | | | | 455.52 (M + H). |
| 105 | 1-acetyl-7-fluoro- 2-(3-methoxy-4- (2-morpholino-2- oxoethoxy)benz- ylidene)indolin-3- one | 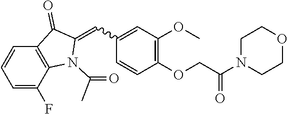 [SMILES: COc1cc(C=C2C(=O)c3cccc(F)c3N2C(C)=O)ccc1OCC(=O)N1CCOCC1] | Z/E Mix (about 3:1) | | CAS No. 1824619- 59-1 (Reagent Supplier 1) CAS No. 31438-76-3 | 1H NMR (270 MHz) δ 8.16- 8.12, 8.07-8.03, 7.73-7.62, 7.53- 7.18 (m, 6H), 7.03-6.92 (m, 1H), 4.82, 4.81 |
| | | | | | (Reagent | (each s, com- |
| | | | | | Supplier 9) | bined 2H), 3.99, |
| | | | | | | 3.89 (each s, |
| | | | | | | combined 3H), |
| | | | | | | 3.72-3.59 (m, |
| | | | | | | 8H), 2.51 (d, J = |
| | | | | | | 5.7 Hz, 0.8H), |
| | | | | | | 2.29 (d, J = 2.7 |
| | | | | | | Hz, 2.2H). MS |
| | | | | | | 455.51 (M + H). |
| 106 | (Z)-N-((1-acetyl- 2-((6-(morpholine- 4-carbonyl)quin- olin-2-yl)meth- ylene)-3-oxoin- dolin-4-yl)meth- | 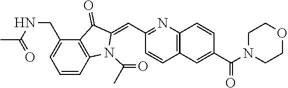 [SMILES: CC(=O)NCc1cccc2c1C(=O)/C(=C/c1ccc3cc(C(=O)N4CCOCC4)ccc3n1)N2C(C)=O] | Z | | Production Ex. 2 Production Ex. 119 | 1H NMR (500 MHz) δ 8.23 (d, J = 8.4 Hz, 1H), 8.13 (d, J = 8.6 Hz, 1H), 8.08 (d, J = 8.3 Hz, 1H), |
| | yl)acetamide | | | | | 7.90 (d, J = 1.9 |
| | | | | | | Hz, 1H), 7.73 |
| | | | | | | (dd, J = 8.7, 1.9 |
| | | | | | | Hz, 1H), 7.64 (d, |
| | | | | | | J = 8.5 Hz, 1H), |
| | | | | | | 7.60-7.55 (m, |
| | | | | | | 1H), 7.30 (s, 1H), |
| | | | | | | 7.26-7.20 (m, |
| | | | | | | 1H), 6.90 (t, J = |
| | | | | | | 6.5 Hz, 1H), 4.68 |
| | | | | | | (d, J = 6.6 Hz, |
| | | | | | | 2H), 3.98-3.39 |
| | | | | | | (m, 8H), 2.12 (s, |
| | | | | | | 3H), 1.96 (s, 3H). |
| | | | | | | MS 499.51 |
| | | | | | | (M + H). |
| 107 | (Z)-1-acetyl-2- ((6-((1,1-dioxido- thiomorpholino)- methyl)quinolin- 2-yl)methylene)- indolin-3-one | 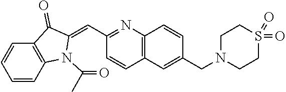 [SMILES: CC(=O)N1/C(=C\c2ccc3cc(CN4CCS(=O)(=O)CC4)ccc3n2)C(=O)c2ccccc21] | Z | | Production Ex. 133 CAS No. 16800-68-3 (Reagent Supplier 3) | 1H NMR (270 MHz) δ 8.23-8.13 (m, 2H), 8.08 (d, J = 8.6 Hz, 1H), 7.84 (dd, J = 7.5, 1.4 Hz, 1H), |
| | | | | | | 7.78-7.58 (m, |
| | | | | | | 4H), 7.34 (s, 1H), |
| | | | | | | 7.29-7.20 (m, |
| | | | | | | 1H), 3.84 (s, 2H), |
| | | | | | | 3.15-3.01 (m, |
| | | | | | | 8H), 2.13 (s, 3H). |
| | | | | | | MS 462.38 |
| | | | | | | (M + H). |
| 108 | (E)-1-acetyl-2- ((6-((E)-3-morph- olino-3-oxoprop- 1-en-1-yl)benzo [d]thiazol-2-yl)- methylene)indolin- 3-one | 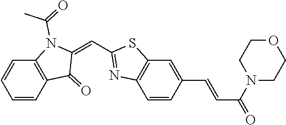 [SMILES: CC(=O)N1/C(=C/c2nc3ccc(/C=C/C(=O)N4CCOCC4)cc3s2)C(=O)c2ccccc21] | E | | Production Ex. 148 CAS No. 16800-68-3 (Reagent Supplier 3) | 1H NMR (500 MHz) δ 8.30 (s, 1H), 8.12-8.04 (m, 3H), 7.93 (dd, J = 7.6, 1.4 Hz, 1H), 7.84 (d, J = 15.4 Hz, 1H), 7.75-7.68 (m, |
| | | | | | | 2H), 7.37-7.31 |
| | | | | | | (m, 1H), 6.96 (d, |
| | | | | | | J = 15.3 Hz, 1H), |
| | | | | | | 3.82-3.65 (m, |
| | | | | | | 8H), 2.76 (s, 3H). |
| | | | | | | MS 460.22 |
| | | | | | | (M + H). |
| 109 | (Z)-1-acetyl-2- ((5-((E)-3-morph- olino-3-oxoprop- 1-en-1-yl)benzo [d]thiazol-2-yl)- methylene)indolin- 3-one | 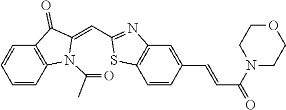 [SMILES: CC(=O)N1/C(=C\c2nc3cc(/C=C/C(=O)N4CCOCC4)ccc3s2)C(=O)c2ccccc21] | Z | | Production Ex. 139 CAS No. 16800-68-3 (Reagent Supplier 3) | 1H NMR (270 MHz) δ 8.29 (d, J = 1.6 Hz, 1H), 8.21 (d, J = 8.3 Hz, 1H), 7.92 (d, J = 8.4 Hz, 1H), 7.88-7.79 (m, |
| | | | | | | 2H), 7.73-7.64 |
| | | | | | | (m, 1H), 7.58 |
| | | | | | | (dd, J = 8.5, 1.7 |
| | | | | | | Hz, 1H), 7.33- |
| | | | | | | 7.26 (m, 2H), |
| | | | | | | 7.02 (d, J = 15.4 |
| | | | | | | Hz, 1H), 3.83- |
| | | | | | | 3.69 (m, 8H), |
| | | | | | | 2.27 (s, 3H). MS |
| | | | | | | 460.39 (M + H). |
| 110 | (E)-1-acetyl-2- ((5-((E)-3-morph- olino-3-oxoprop- 1-en-1-yl)benzo [d]thiazol-2-yl)- methylene)indolin- 3-one | 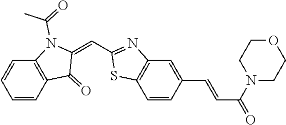 [SMILES: CC(=O)N1/C(=C/c2nc3cc(/C=C/C(=O)N4CCOCC4)ccc3s2)C(=O)c2ccccc21] | E | | Production Ex. 139 CAS No. 16800-68-3 (Reagent Supplier 3) | 1H NMR (270 MHz) δ 8.30 (s, 1H), 8.22 (d, J = 1.5 Hz, 1H), 8.07 (d, J = 8.3 Hz, 1H), 7.97 (d, J = 8.4 Hz, 1H), 7.92 (dd, J = 7.6, 1.5 |
| | | | | | | Hz, 1H), 7.86 (d, |
| | | | | | | J = 15.3 Hz, 1H), |
| | | | | | | 7.75-7.67 (m, |
| | | | | | | 1H), 7.65 (dd, J = |
| | | | | | | 8.5, 1.6 Hz, 1H), |
| | | | | | | 7.38-7.29 (m, |
| | | | | | | 1H), 6.97 (d, J = |
| | | | | | | 15.3 Hz, 1H), |
| | | | | | | 3.83-3.64 (m, |
| | | | | | | 8H), 2.76 (s, 3H). |
| | | | | | | MS 460.39 |
| | | | | | | (M + H). |
| 111 | (Z)-2-((1-acetyl- 3-oxoindolin-2- ylidene)methyl)- 6-(morpholine-4- carbonyl)quin- oline-4-carbox- amide | 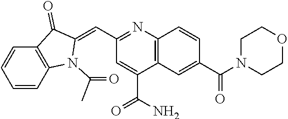 [SMILES: CC(=O)N1/C(=C\c2cc(C(N)=O)c3cc(C(=O)N4CCOCC4)ccc3n2)C(=O)c2ccccc21] | Z | | Production Ex. 182 CAS No. 16800-68-3 (Reagent Supplier 3) | 1H NMR (270 MHz) δ 8.34 (d, J = 1.8 Hz, 1H), 8.12 (d, J = 8.3 Hz, 1H), 8.05 (d, J = 8.7 Hz, 1H), 7.83 (dd, J = 7.6, 1.4 Hz, 1H), |
| | | | | | | 7.71-7.62 (m, |
| | | | | | | 3H), 7.26 (t, J = |
| | | | | | | 7.4 Hz, 1H), 7.17 |
| | | | | | | (s, 1H), 6.85 (br |
| | | | | | | s, 1H), 6.18 (br s, |
| | | | | | | 1H), 3.92-3.39 |
| | | | | | | (m, 8H), 2.11 (s, |
| | | | | | | 3H). MS 471.3 |
| | | | | | | (M + H). |
| 112 | (Z)-2-((4-((3H- [1,2,3]triazolo [4,5-b]pyridin-3- yl)oxy)-6-(morph- oline-4-carbonyl)- quinolin-2-yl)- methylene)-1-acet- ylindolin-3-one | 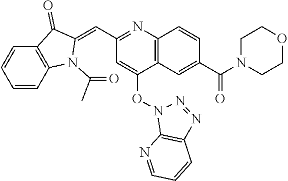 [SMILES: CC(=O)N1/C(=C\c2cc(On3nnc4cccnc43)c3cc(C(=O)N4CCOCC4)ccc3n2)C(=O)c2ccccc21] | Z | Ex. 111 | Production Ex. 152 CAS No. 16800-68-3 [symbol] 3 | 1H NMR (270 MHz) δ 8.80 (dd, J = 4.5, 1.5 Hz, 1H), 8.64-8.51 (m, 2H), 8.20 (d, J = 8.7 Hz, 1H), 8.11 (d, J = 8.3 Hz, 1H), 7.89 (dd, J = 8.8, 1.8 Hz, 1H), 7.81- 7.75 (m, 1H), |
| | | | | | | 7.68-7.54 (m, |
| | | | | | | 2H), 7.28-7.17 |
| | | | | | | (m, 1H), 6.94 (s, |
| | | | | | | 1H), 6.53 (s, 1H), |
| | | | | | | 3.96-3.46 (m, |
| | | | | | | 8H), 2.12 (s, 3H). |
| | | | | | | MS 562.3 |
| | | | | | | (M + H). |
| 113 | (Z)-N-(2-((1- acetyl-3-oxoin- dolin-2-ylidene)- methyl)-6-(morph- oline-4-carbonyl)- quinolin-4-yl)- acetamide | 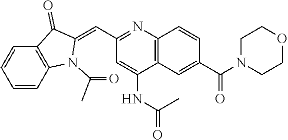 [SMILES: CC(=O)Nc1cc(/C=C2/C(=O)c3ccccc3N2C(C)=O)nc2ccc(C(=O)N3CCOCC3)cc12] | Z | Ex. 111 | Production Ex. 181 CAS No. 16800-68-3 (Reagent Supplier 3) | 1H NMR (270 MHz) δ 8.90 (br s,1H), 8.49 (s, 1H), 8.16-8.06 (m, 2H), 7.95 (d, J = 8.6 Hz, 1H), 7.82 (dd, J = 7.6, 1.4 Hz, 1H), 7.67-7.52 (m, |
| | | | | | | 2H), 7.28 (s, 1H), |
| | | | | | | 7.27-7.17 (m, |
| | | | | | | 1H), 3.97-3.35 |
| | | | | | | (m, 8H), 2.35 (s, |
| | | | | | | 3H), 2.07 (s, 3H). |
| | | | | | | MS 485.28 |
| | | | | | | (M + H). |
| 114 | (Z)-1-acetyl-2-((6- (4-phenylpiper- idine-1-carbonyl)- quinolin-2-yl)meth- ylene)indolin-3-one | 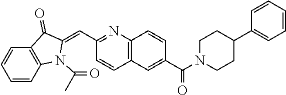 [SMILES: CC(=O)N1/C(=C\c2ccc3cc(C(=O)N4CCC(c5ccccc5)CC4)ccc3n2)C(=O)c2ccccc21] | Z | | Production Ex. 128 CAS No. 16800-68-3 (Reagent Supplier 3) | 1H NMR (270 MHz) δ 8.24 (d, J = 8.6 Hz, 1H), 8.19 (d, J = 8.3 Hz, 1H), 8.15 (d, J = 8.6 Hz, 1H), |
| | | | | | | 7.93 (d, J = 1.8 |
| | | | | | | Hz, 1H), 7.85 |
| | | | | | | (dd, J = 7.5, 1.3 |
| | | | | | | Hz, 1H), 7.79 |
| | | | | | | (dd, J = 8.6, 1.9 |
| | | | | | | Hz, 1H), 7.70- |
| | | | | | | 7.61 (m, 2H), |
| | | | | | | 7.39-7.33 (m, |
| | | | | | | 2H), 7.30-7.20 |
| | | | | | | (m, 5H), 5.06- |
| | | | | | | 4.83 (m, 1H), |
| | | | | | | 4.03-3.83 (m, |
| | | | | | | 1H), 3.36-2.70 |
| | | | | | | (m, 3H), 2.14 (s, |
| | | | | | | 3H), 2.10-1.50 |
| | | | | | | (m, 4H). MS |
| | | | | | | 502.55 (M + H). |
| 115 | (Z)-1-acetyl-2-((6- (4-morpholino- piperidine-1- carbonyl)quinolin- 2-yl)methylene)- indolin-3-one | 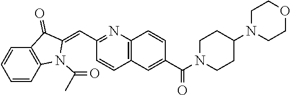 [SMILES: CC(=O)N1/C(=C\c2ccc3cc(C(=O)N4CCC(N5CCOCC5)CC4)ccc3n2)C(=O)c2ccccc21] | Z | | Production Ex. 129 CAS No. 16800-68-3 (Reagent Supplier 3) | 1H NMR (270 MHz) δ 8.23 (d, J = 8.4 Hz, 1H), 8.19 (d, J = 8.3 Hz, 1H), 8.13 (d, J = 8.7 Hz, 1H), |
| | | | | | | 7.89 (d, J = 1.8 |
| | | | | | | Hz, 1H), 7.85 |
| | | | | | | (dd, J = 7.6, 1.4 |
| | | | | | | Hz, 1H), 7.73 |
| | | | | | | (dd, J = 8.7, 1.8 |
| | | | | | | Hz, 1H), 7.71- |
| | | | | | | 7.61 (m, 2H), |
| | | | | | | 7.34 (s, 1H), |
| | | | | | | 7.29-7.21 (m, |
| | | | | | | 1H), 4.88-4.65 |
| | | | | | | (m, 1H), 3.94- |
| | | | | | | 3.65 (m, 5H), |
| | | | | | | 3.20-2.80 (m, |
| | | | | | | 2H), 2.63-2.41 |
| | | | | | | (m, 5H), 2.14 (s, |
| | | | | | | 3H), 2.10-1.74 |
| | | | | | | (m, 4H). MS |
| | | | | | | 511.55 (M + H). |
| 116 | (Z)-1-acetyl-2-((6- (4-(2-methoxy- ethyl)piperazine-1- carbonyl)quinolin- 2-yl)methylene)- indolin-3-one | 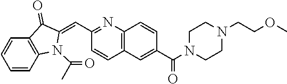 [SMILES: COCCN1CCN(C(=O)c2ccc3nc(/C=C4/C(=O)c5ccccc5N4C(C)=O)ccc3c2)CC1] | Z | Ex. 115 | Production Ex. 130 CAS No. 16800-68-3 (Reagent Supplier 3) | 1H NMR (270 MHz) δ 8.23 (d, J = 8.4 Hz, 1H), 8.19 (d, J = 8.3 Hz, 1H), 8.13 (d, J = 8.7 Hz, 1H), |
| | | | | | | 7.90 (d, J = 1.8 |
| | | | | | | Hz, 1H), 7.84 |
| | | | | | | (dd, J = 7.7, 1.4 |
| | | | | | | Hz, 1H), 7.75 |
| | | | | | | (dd, J = 8.6, 1.9 |
| | | | | | | Hz, 1H), 7.71- |
| | | | | | | 7.60 (m, 2H), |
| | | | | | | 7.33 (s, 1H), |
| | | | | | | 7.30-7.21 (m, |
| | | | | | | 1H), 3.96-3.43 |
| | | | | | | (m, 6H), 3.36 (s, |
| | | | | | | 3H), 2.71-2.42 |
| | | | | | | (m, 6H), 2.13 (s, |
| | | | | | | 3H). MS 485.50 |
| | | | | | | (M + H). |
| 117 | (E)-1-acetyl-2-((6- (morpholine-4- carbonyl)-[4,4′- biquinolin]-2-yl)- methylene)indolin- 3-one | 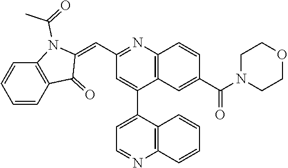 [SMILES: CC(=O)N1/C(=C/c2cc(-c3ccnc4ccccc34)c3cc(C(=O)N4CCOCC4)ccc3n2)C(=O)c2ccccc21] | E | | Production Ex. 102 CAS No. 16800-68-3 (Reagent Supplier 3) | 1H NMR (500 MHz) δ 9.10 (d, J = 4.3 Hz, 1H), 8.30-8.21 (m, 2H), 8.13 (s, 1H), 8.08-8.03 (m, 1H), 7.99 (s, 1H), 7.82-7.72 (m, 3H), 7.70-7.64 (m, 1H), 7.59 (dd, J = 8.4, 1.4 |
| | | | | | | Hz, 1H), 7.56 (d, |
| | | | | | | J = 4.3 Hz, 1H), |
| | | | | | | 7.52-7.46 (m, |
| | | | | | | 1H), 7.41 (d, J = |
| | | | | | | 1.8 Hz, 1H), |
| | | | | | | 7.29-7.22 (m, |
| | | | | | | 1H), 3.79-3.11 |
| | | | | | | (m, 8H), 2.75 (s, |
| | | | | | | 3H). MS 555.34 |
| | | | | | | (M + H). |
| 118 | 1-acetyl-2-((6- (morpholine-4- carbonyl)-4-(naph- thalen-1-yl)quin- olin-2-yl)methyl- ene)indolin-3-one | 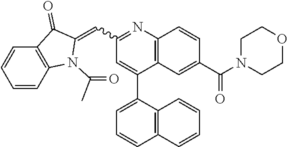 [SMILES: CC(=O)N1C(=Cc2cc(-c3cccc4ccccc34)c3cc(C(=O)N4CCOCC4)ccc3n2)C(=O)c2ccccc21] | Z/E Mix. (about 88:12) | Ex. 117 | Production Ex. 103 CAS No. 16800-68-3 (Reagent Supplier 3) | 1H NMR (500 MHz) δ 8.29-8.17 (m, 2H), 8.07- 7.95 (m, 2H), 7.87-7.75 (m, 2H), 7.70-7.59 (m, 3H), 7.57- 7.47 (m, 2H), 7.40-7.21 (m, |
| | | | | | | 5H), 3.79-3.05 |
| | | | | | | (m, 8H), 2.74, |
| | | | | | | 2.28 (each s, |
| | | | | | | combined 3H). |
| | | | | | | MS 554.34 |
| | | | | | | (M + H). |
| 119 | (Z)-1-acetyl-2-((6- (4-((3-methoxy- oxetan-3-yl)meth- yl)piperazine-1- carbonyl)quinolin- 2-yl)methylene)- | 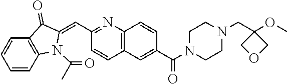 [SMILES: COC1(CN2CCN(C(=O)c3ccc4nc(/C=C5/C(=O)c6ccccc6N5C(C)=O)ccc4c3)CC2)COC1] | Z | | Production Ex. 58 CAS No. 577354-59-6 (Reagent Supplier 1) | 1H NMR (270 MHz) δ 8.26- 8.16 (m, 2H), 8.13 (d, J = 8.7 Hz, 1H), 7.90- 7.81 (m, 2H), |
| | indolin-3-one | | | | CAS No. | 7.73 (dd, J = 8.7, |
| | | | | | 16800-68-3 | 1.8 Hz, 1H), |
| | | | | | (Reagent | 7.70-7.59 (m, |
| | | | | | Supplier 3) | 2H), 7.33 (s, 1H), |
| | | | | | | 7.29-7.21 (m, |
| | | | | | | 1H), 4.71 (d, J = |
| | | | | | | 6.9 Hz, 2H), 4.44 |
| | | | | | | (d, J = 6.9 Hz, |
| | | | | | | 2H), 3.94-3.39 |
| | | | | | | (m, 4H), 3.35 |
| | | | | | | (s, 3H), 2.86 (s, |
| | | | | | | 2H), 2.75-2.44 |
| | | | | | | (m, 4H), 2.13 (s, |
| | | | | | | 3H). MS 527.51 |
| | | | | | | (M + H). |
| 120 | (3-methoxyoxetan- 3-yl)methyl (Z)-4- (2-((1-acetyl-3- oxoindolin-2-yl- idene)methyl)quin- | 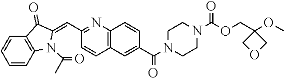 [SMILES: COC1(COC(=O)N2CCN(C(=O)c3ccc4nc(/C=C5/C(=O)c6ccccc6N5C(C)=O)ccc4c3)CC2)COC1] | Z | | Production Ex. 58 CAS No. 577354- 59-6 | 1H NMR (270 MHz) δ 8.26- 8.07 (m, 3H), 7.90 (d, J = 1.8 Hz, 1H), 7.85 |
| | oline-6-carbonyl)- | | | | (Reagent | (dd, J = 7.5, 1.3 |
| | piperazine-1-car- | | | | Supplier 1) | Hz, 1H), 7.73 |
| | boxylate | | | | CAS No. | (dd, J = 8.6, 1.9 |
| | | | | | 16800-68-3 | Hz, 1H), 7.70- |
| | | | | | (Reagent | 7.62 (m, 2H), |
| | | | | | Supplier 3) | 7.33 (s, 1H), |
| | | | | | | 7.29-7.21 (m, |
| | | | | | | 1H), 4.70 (d, |
| | | | | | | J = 7.0 Hz, 2H), |
| | | | | | | 4.50-4.41 (m, |
| | | | | | | 4H), 3.90-3.40 |
| | | | | | | (m, 8H), 3.34 (s, |
| | | | | | | 3H), 2.14 (s, |
| | | | | | | 3H). MS 571.50 |
| | | | | | | (M + H). |
| 122 | 2-(3-methoxy-4- (2-morpholino-2- oxoethoxy)benz- ylidene)-1-methyl- indolin-3-one | 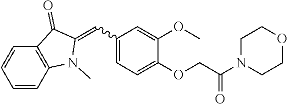 [SMILES: COc1cc(C=C2C(=O)c3ccccc3N2C)ccc1OCC(=O)N1CCOCC1] | Z/E Mix. (about 1:3) | | Production Ex. 89 | 1H NMR (270 MHz) δ 8.60 (d, J = 2.2 Hz, 0.8H), 7.77-7.67 (m, 1H), 7.58-7.42 (m, 1H), 7.26- 7.21 (m, 0.4H), |
| | | | | | | 7.04-6.81 (m, |
| | | | | | | 4H), 6.31 (s, |
| | | | | | | 0.8H), 4.81, |
| | | | | | | 4.80 (each s, |
| | | | | | | combined 2H), |
| | | | | | | 4.03, 3.91 (each |
| | | | | | | s, combined |
| | | | | | | 3H), 3.71-3.59 |
| | | | | | | (m, 8H), 3.36, |
| | | | | | | 3.16 (each s, |
| | | | | | | combined 3H). |
| | | | | | | MS 409.23 |
| | | | | | | (M + H). |
| 123 | ethyl 2-(3-meth- oxy-4-(2-morph- olino-2-oxoeth- oxy)benzylidene)- 3-oxoindolin-1- carboxylate | 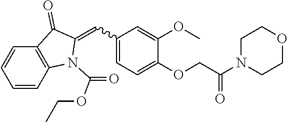 [SMILES: CCOC(=O)N1C(=Cc2ccc(OCC(=O)N3CCOCC3)c(OC)c2)C(=O)c2ccccc21] | Z/E Mix. (about 3:1) | | Production Ex. 89 | 1H NMR (270 MHz) δ 8.22- 7.99 (m, 2H), 7.91-7.76 (m, 1H), 7.74-7.56 (m, 1H), 7.38- 7.19 (m, 2H), 7.10-6.92 (m, |
| | | | | | | 2H), 4.82, 4.81 |
| | | | | | | (each s, com- |
| | | | | | | bined 2H), 4.51, |
| | | | | | | 4.00 (each q, |
| | | | | | | each J = 7.1 Hz, |
| | | | | | | combined 2H), |
| | | | | | | 3.99, 3.88 (each |
| | | | | | | s, combined |
| | | | | | | 3H), 3.72-3.60 |
| | | | | | | (m, 8H), 1.51, |
| | | | | | | 0.89 (each t, |
| | | | | | | each J = 7.1 Hz, |
| | | | | | | combined 3H). |
| | | | | | | MS 467.26 |
| | | | | | | (M + H). |
| 124 | 2-(3-methoxy-4- (2-morpholino-2- oxoethoxy)benz- ylidene)-1-(methyl- sulfonyl)indolin- 3-one | 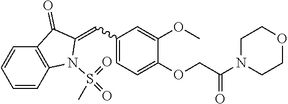 [SMILES: COc1cc(C=C2C(=O)c3ccccc3N2S(C)(=O)=O)ccc1OCC(=O)N1CCOCC1] | Z/E Mix. (about 2:3) | | Production Ex. 89 | 1H NMR (270 MHz) δ 8.02- 7.83 (m, 3H), 7.81-7.61 (m, 1H), 7.54-7.29 (m, 3H), 7.01- 6.93 (m, 1H), |
| | | | | | | 4.85, 4.82 (each |
| | | | | | | s, combined 2H), |
| | | | | | | 4.01, 3.95 (each |
| | | | | | | s, combined 3H), |
| | | | | | | 3.71-3.61 (m, |
| | | | | | | 8H), 2.88, 2.56 |
| | | | | | | (each s, com- |
| | | | | | | bined 3H). MS |
| | | | | | | 473.16 (M + H). |
| 125 | 1-acetyl-2-(4- (2-hydroxyeth- oxy)-3-ethoxy- benzylidene)in- dolin-3-one | 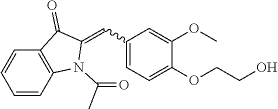 [SMILES: COc1cc(C=C2C(=O)c3ccccc3N2C(C)=O)ccc1OCCO] | Z/E Mix. (about 5:1) | | Production Ex. 195 | 1H NMR (270 MHz) δ 8.31- 8.24, 8.15-8.08, 7.72-7.59, 7.49- 7.06 (all m, com- bined 6H), 7.86, 7.81 (each dd, |
| | | | | | | each J = 7.6, 1.4 |
| | | | | | | Hz, combined |
| | | | | | | 1H), 6.96, 6.94 |
| | | | | | | (each d, each J = |
| | | | | | | 8.4 Hz, com- |
| | | | | | | bined 1H), 4.24- |
| | | | | | | 4.13 (m, 2H), |
| | | | | | | 4.07-3.97 (m, |
| | | | | | | 2H), 4.00, 3.88 |
| | | | | | | (each s, com- |
| | | | | | | bined 3H), 2.64, |
| | | | | | | 2.05 (each s, |
| | | | | | | combined 3H). |
| | | | | | | MS 354.21 |
| | | | | | | (M + H). |
| 126 | (Z)-1-acetyl-2-((6- (2-hydroxyeth- oxy)quinolin-2- yl)methylene)in- dolin-3-one | 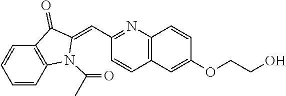 [SMILES: CC(=O)N1/C(=C\c2ccc3cc(OCCO)ccc3n2)C(=O)c2ccccc21] | Z | | Production Ex. 194 | 1H NMR (270 MHz) δ 8.21 (d, J = 8.3 Hz, 1H), 8.09-7.98 (m, 2H), 7.84 (dd, J = 7.6, 1.4 Hz, |
| | | | | | | 1H), 7.69-7.59 |
| | | | | | | (m, 1H), 7.55 (d, |
| | | | | | | J = 8.6 Hz, 1H), |
| | | | | | | 7.42 (dd, J = 9.3, |
| | | | | | | 2.8 Hz, 1H), 7.33 |
| | | | | | | (s, 1H), 7.24 (t, |
| | | | | | | J = 7.5 Hz, 1H), |
| | | | | | | 7.08 (d, J = 2.7 |
| | | | | | | Hz, 1H), 4.27- |
| | | | | | | 4.19 (m, 2H), |
| | | | | | | 4.11-4.02 (m, |
| | | | | | | 2H), 2.12 (s, 3H). |
| | | | | | | MS 375.34 |
| | | | | | | (M + H). |
| 127 | (Z)-2-((1-acetyl-3- oxoindolin-2-yl- idene)methyl)quin- oline-6-carboxylic acid | 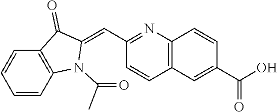 [SMILES: CC(=O)N1/C(=C\c2ccc3cc(C(=O)O)ccc3n2)C(=O)c2ccccc21] | Z | | Production Ex. 199 | 1H NMR (270 MHz, dimethyl- sulfoxide-d6) δ 8.68 (d, J = 1.9 Hz, 1H), 8.65 (d, J = 8.5 Hz, 1H), 8.27 (dd, J = 8.9, |
| | | | | | | 2.0 Hz, 1H), |
| | | | | | | 8.10-7.93 (m, |
| | | | | | | 3H), 7.88-7.73 |
| | | | | | | (m, 2H), 7.39 (s, |
| | | | | | | 1H), 7.34 (t, J = |
| | | | | | | 7.5 Hz, 1H), 2.09 |
| | | | | | | (s, 3H). MS |
| | | | | | | 359.29 (M + H). |
| 128 | (Z)-4-([1,1′- biphenyl]-2-yl)-2- ((1-acetyl-3-oxo- indolin-2-ylidene)- methyl)quinoline- 6-carboxylic acid | 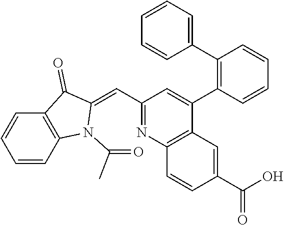 [SMILES: CC(=O)N1/C(=C\c2cc(-c3ccccc3-c3ccccc3)c3cc(C(=O)O)ccc3n2)C(=O)c2ccccc21] | Z | Ex. 127 | Production Ex. 204 | 1H NMR (500 MHz) δ 8.42 (d, J = 1.9 Hz, 1H), 8.21 (dd, J = 8.8, 1.9 Hz, 1H), 8.14 (d, J = 8.8 Hz, 1H), 8.06 (d, J = 8.4 Hz, 1H), 7.86-7.81 (m, 1H), 7.74-7.53 (m, 4H), 7.50- 7.43 (m, 2H), 7.32-7.24 (m, 1H), 7.22 (s, |
| | | | | | | 1H), 7.06-6.98 |
| | | | | | | (m, 5H), 6.81 (br |
| | | | | | | s, 1H), 2.14 (s, |
| | | | | | | 3H). MS 511.31 |
| | | | | | | (M + H) |
| 129 | (Z)-1-(2-((1-acet- yl-3-oxoindolin- 2-ylidene)meth- yl)quinoline-6- carbonyl)piperi- dine-4-carboxylic | 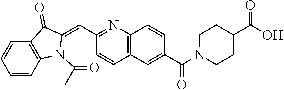 [SMILES: CC(=O)N1/C(=C\c2ccc3cc(C(=O)N4CCC(C(=O)O)CC4)ccc3n2)C(=O)c2ccccc21] | Z | | Production Ex. 200 | 1H NMR (270 MHz) δ 8.26 (d, J = 8.5 Hz, 1H), 8.20-8.11 (m, 2H), 7.91 (d, J = 1.8 Hz, 1H), 7.86 |
| | acid | | | | | (dd, J = 7.6, 1.4 |
| | | | | | | Hz, 1H), 7.74 |
| | | | | | | (dd, J = 8.7, 1.8 |
| | | | | | | Hz, 1H), 7.71- |
| | | | | | | 7.63 (m, 2H), |
| | | | | | | 7.35 (s, 1H), |
| | | | | | | 7.31-7.23 (m, |
| | | | | | | 1H), 4.71-4.40 |
| | | | | | | (m, 1H), 3.94- |
| | | | | | | 3.65 (m, 1H), |
| | | | | | | 3.28-3.08 (m, |
| | | | | | | 2H), 2.76-2.60 |
| | | | | | | (m, 1H), 2.23- |
| | | | | | | 1.63 (m, 4H), |
| | | | | | | 2.16 (s, 3H). MS |
| | | | | | | 470.25 (M + H). |
| 130 | (E)-1-acetyl-2- ((5-(((tetrahydro- 2H-pyran-4-yl)- amino)methyl)- benzo[d]thiazol- 2-yl)methylene)- indolin-3-one | 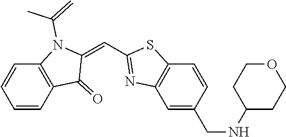 [SMILES: C=C(C)N1/C(=C/c2nc3cc(CNC4CCOCC4)ccc3s2)C(=O)c2ccccc21] | E | | Production Ex. 206 | 1H NMR (270 MHz) δ 8.26 (s, 1H), 8.10 (d, J = 8.3 Hz, 1H), 8.04 (d, J = 1.5 Hz, 1H), 7.97-7.87 (m, 2H), 7.74- 7.65 (m, 1H), 7.49 (dd, J = 8.2, |
| | | | | | | 1.6 Hz, 1H), 7.33 |
| | | | | | | (t, J = 7.5 Hz, |
| | | | | | | 1H), 4.06-3.92 |
| | | | | | | (m, 4H), 3.45- |
| | | | | | | 3.31 (m, 2H), |
| | | | | | | 2.85-2.68 (m, |
| | | | | | | 1H), 2.75 (s, |
| | | | | | | 3H), 1.95-1.83 |
| | | | | | | (m, 2H), 1.55- |
| | | | | | | 1.39 (m, 2H). MS |
| | | | | | | 434.28 (M + H). |
| 131 | (Z)-1-acetyl-2- ((6-(((tetrahydro- 2H-pyran-4-yl)- amino)methyl)- quinolin-2-yl)- methylene)in- dolin-3-one | 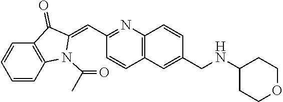 [SMILES: CC(=O)N1/C(=C\c2ccc3cc(CNC4CCOCC4)ccc3n2)C(=O)c2ccccc21] | Z | Ex. 130 | Production Ex. 196 | 1H NMR (270 MHz) δ 8.21 (d, J = 8.3 Hz, 1H), 8.16 (d, J = 8.5 Hz, 1H), 8.07 (d, J = 8.6 Hz, 1H), 7.84 (dd, J = 7.7, |
| | | | | | | 1.5 Hz, 1H), |
| | | | | | | 7.78-7.56 (m, |
| | | | | | | 4H), 7.34 (s, |
| | | | | | | 1H), 7.29-7.18 |
| | | | | | | (m, 1H), 4.06- |
| | | | | | | 3.94 (m, 4H), |
| | | | | | | 3.47-3.33 (m, |
| | | | | | | 2H), 2.86-2.71 |
| | | | | | | (m, 1H), 2.12 |
| | | | | | | (s, 3H), 1.97- |
| | | | | | | 1.85 (m, 2H), |
| | | | | | | 1.59-1.41 (m, |
| | | | | | | 2H). MS 428.46 |
| | | | | | | (M + H). |
| 132 | (Z)-1-acetyl-2- ((5-(((tetrahydro- 2H-pyran-4-yl)- amino)methyl)- benzo[d]thiazol- 2-yl)methylene)- indolin-3-one | 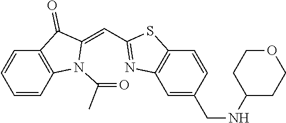 [SMILES: CC(=O)N1/C(=C\c2nc3cc(CNC4CCOCC4)ccc3s2)C(=O)c2ccccc21] | Z | Ex. 130 | Production Ex. 205 | 1H NMR (270 MHz) δ 8.22 (d, J = 8.3 Hz, 1H), 8.07 (d, J = 1.4 Hz, 1H), 7.88 (d, J = 8.3 Hz, 1H), 7.84 (dd, J = 7.7, 1.3 Hz, 1H), |
| | | | | | | 7.72-7.63 (m, |
| | | | | | | 1H), 7.48 (dd, |
| | | | | | | J = 8.3, 1.6 Hz, |
| | | | | | | 1H), 7.34-7.24 |
| | | | | | | (m, 2H), 4.06- |
| | | | | | | 3.93 (m, 4H), |
| | | | | | | 3.47-3.34 (m, |
| | | | | | | 2H), 2.84-2.69 |
| | | | | | | (m, 1H), 2.25 |
| | | | | | | (s, 3H), 1.95- |
| | | | | | | 1.83 (m, 2H), |
| | | | | | | 1.57-1.39 (m, |
| | | | | | | 2H). MS 434.27 |
| | | | | | | (M + H). |
| 133 | (Z)-1-acetyl-2- ((6-(((1,1-dioxido- tetrahydro-2H- thiopyran-4-yl)- amino)methyl)- quinolin-2-yl)- methylene)indolin- | 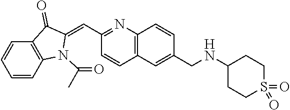 [SMILES: CC(=O)N1/C(=C\c2ccc3cc(CNC4CCS(=O)(=O)CC4)ccc3n2)C(=O)c2ccccc21] | Z | Ex. 130 | Production Ex. 201 | 1H NMR (270 MHz) δ 8.23-8.14 (m, 2H), 8.08 (d, J = 8.6 Hz, 1H), 7.84 (dd, J = 7.6, 1.4 Hz, 1H), 7.77-7.58 (m, |
| | 3-one | | | | | 4H), 7.34 (s, 1H), |
| | | | | | | 7.29-7.21 (m, |
| | | | | | | 1H), 3.98 (s, 2H), |
| | | | | | | 3.43-3.29 (m, |
| | | | | | | 2H), 3.06-2.96 |
| | | | | | | (m, 1H), 2.96- |
| | | | | | | 2.83 (m, 2H), |
| | | | | | | 2.40-2.23 (m, |
| | | | | | | 2H), 2.22-2.05 |
| | | | | | | (m, 2H), 2.13 (s, |
| | | | | | | 3H). MS 476.27 |
| | | | | | | (M + H). |
| 134 | (Z)-1-acetyl-2- ((6-(((tetrahydro- 2H-pyran-4-yl)- amino)methyl)- benzo[d]thiazol- 2-yl)methylene)- | 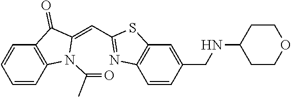 [SMILES: CC(=O)N1/C(=C\c2nc3ccc(CNC4CCOCC4)cc3s2)C(=O)c2ccccc21] | Z | Ex. 130 | Production Ex. 207 | 1H NMR (270 MHz) δ 8.22 (d, J = 8.2 Hz, 1H), 8.06 (d, J = 8.5 Hz, 1H), 7.93 (d, J = 1.6 Hz, 1H), |
| | indolin-3-one | | | | | 7.84 (dd, J = 7.6, |
| | | | | | | 1.5 Hz, 1H), |
| | | | | | | 7.72-7.63 (m, |
| | | | | | | 1H), 7.51 (dd, |
| | | | | | | J = 8.4, 1.7 Hz, |
| | | | | | | 1H), 7.32 (s, 1H), |
| | | | | | | 7.32-7.24 (m, |
| | | | | | | 1H), 4.05-3.94 |
| | | | | | | (m, 4H), 3.47- |
| | | | | | | 3.33 (m, 2H), |
| | | | | | | 2.84-2.70 (m, |
| | | | | | | 1H), 2.24 (s, 3H), |
| | | | | | | 1.95-1.84 (m, |
| | | | | | | 2H), 1.55-1.38 |
| | | | | | | (m, 2H). MS |
| | | | | | | 434.34 (M + H). |
| 135 | (E)-1-acetyl-2- ((6-(((tetrahydro- 2H-pyran-4-yl)- amino)methyl)- benzo[d]thiazol- 2-yl)methylene)- indolin-3-one | 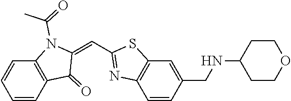 [SMILES: CC(=O)N1/C(=C/c2nc3ccc(CNC4CCOCC4)cc3s2)C(=O)c2ccccc21] | E | Ex. 130 | Production Ex. 208 | 1H NMR (270 MHz) δ 8.24 (s, 1H), 8.11 (d, J = 8.4 Hz, 1H), 8.05 (d, J = 8.5 Hz, 1H), 7.96 (d, J = 1.6 Hz, 1H), 7.92 |
| | | | | | | (dd, J = 7.8, 1.5 |
| | | | | | | Hz, 1H), 7.74- |
| | | | | | | 7.66 (m, 1H), |
| | | | | | | 7.52 (dd, J = 8.4, |
| | | | | | | 1.7 Hz, 1H), 7.33 |
| | | | | | | (t, J = 7.5 Hz, |
| | | | | | | 1H), 4.05-3.94 |
| | | | | | | (m, 4H), 3.47- |
| | | | | | | 3.34 (m, 2H), |
| | | | | | | 2.85-2.70 (m, |
| | | | | | | 1H), 2.75 (s, |
| | | | | | | 3H), 1.96-1.84 |
| | | | | | | (m, 2H), 1.58- |
| | | | | | | 1.41 (m, 2H). |
| | | | | | | MS 434.33 |
| | | | | | | (M + H). |
| 136 | (Z)-1-acetyl-2- ((6-((((2SR,6RS)- 2,6-dimethyltetra- hydro-2H-pyran- 4-yl)amino)meth- yl)quinolin-2-yl)- methylene)indolin- 3-one | 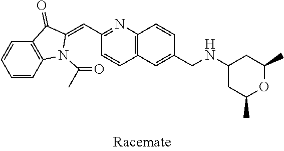 [SMILES: CC(=O)N1/C(=C\c2ccc3cc(CNC4C[C@@H](C)O[C@@H](C)C4)ccc3n2)C(=O)c2ccccc21] | Z | Ex. 130 | Production Ex. 197 | 1H NMR (270 MHz) δ 8.24-8.13 (m, 2H), 8.07 (d, J = 8.6 Hz, 1H), 7.84 (dd, J = 7.6, 1.4 Hz 1H), 7.78-7.62 (m, 3H), 7.59 (d, J = 8.5 Hz, 1H), 7.34 (s, 1H), 7.28- |
| | | | | | | 7.20 (m, 1H), |
| | | | | | | 4.03 (s, 2H), |
| | | | | | | 3.55-3.40 (m, |
| | | | | | | 2H), 2.87-2.71 |
| | | | | | | (m, 1H), 2.12 (s, |
| | | | | | | 3H), 2.01-1.88 |
| | | | | | | (m, 2H), 1.23 (d, |
| | | | | | | J = 6.2 Hz, 6H), |
| | | | | | | 1.14-0.98 (m, |
| | | | | | | 2H). MS 456.36 |
| | | | | | | (M + H). |
| 137 | (Z)-1-acetyl-2- ((6-((((2SR,6SR)- 2,6-dimethyltetra- hydro-2H-pyran- 4-yl)amino)meth- yl)quinolin-2-yl)- methylene)indolin- 3-one | 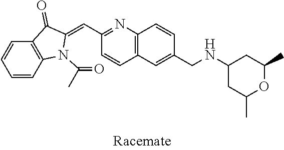 [SMILES: CC(=O)N1/C(=C\c2ccc3cc(CNC4CC(C)O[C@H](C)C4)ccc3n2)C(=O)c2ccccc21] | Z | Ex. 130 | Production Ex. 198 | 1H NMR (270 MHz) δ 8.21 (d, J = 8.3 Hz, 1H), 8.16 (d, J = 8.5 Hz, 1H), 8.06 (d, J = 8.5 Hz, 1H), 7.84 (dd, J = 7.6, 1.4 Hz, 1H), 7.78-7.55 (m, 4H), 7.34 (s, |
| | | | | | | 1H), 7.24 (t, J = |
| | | | | | | 7.4 Hz, 1H), |
| | | | | | | 4.05-3.89 (m, |
| | | | | | | 4H), 3.18-3.09 |
| | | | | | | (m, 1H), 2.12 (s, |
| | | | | | | 3H), 1.70-1.60 |
| | | | | | | (m, 2H), 1.50- |
| | | | | | | 1.36 (m, 2H), |
| | | | | | | 1.17 (d, J = 6.2 |
| | | | | | | Hz, 6H). MS |
| | | | | | | 456.36 (M + H). |
| 138 | (Z)-1-acetyl-2- ((6-(((1-(oxetan- 3-yl)piperidin-4- yl)amino)meth- yl)quinolin-2-yl)- methylene)indolin- 3-one | 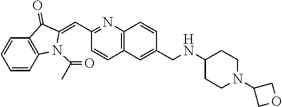 [SMILES: CC(=O)N1/C(=C\c2ccc3cc(CNC4CCN(C5COC5)CC4)ccc3n2)C(=O)c2ccccc21] | Z | | Production Ex. 203 | 1H NMR (270 MHz) δ 8.21- 8.11 (m, 2H), 8.05 (d, J = 8.6 Hz, 1H), 7.84 (dd, J = 7.6, 1.5 Hz, 1H), 7.81- |
| | | | | | | 7.71 (m, 2H), |
| | | | | | | 7.70-7.61 (m, |
| | | | | | | 1H), 7.56 (d, J = |
| | | | | | | 8.5 Hz, 1H), |
| | | | | | | 7.29 (s, 1H), |
| | | | | | | 7.28-7.20 (m, |
| | | | | | | 1H), 4.65-4.54 |
| | | | | | | (m, 4H), 4.03 |
| | | | | | | (s, 2H), 3.50- |
| | | | | | | 3.37 (m, 1H), |
| | | | | | | 2.76-2.59 (m, |
| | | | | | | 3H), 2.10 (s, |
| | | | | | | 3H), 2.04-1.92 |
| | | | | | | (m, 2H), 1.91- |
| | | | | | | 1.78 (m, 2H), |
| | | | | | | 1.68-1.50 (m, |
| | | | | | | 2H). MS 483.51 |
| | | | | | | (M + H). |
| 139 | 1-acetyl-2-((6- (piperazine-1- carbonyl)quinolin- 2-yl)methylene)- indolin-3-one dihydrochloride | 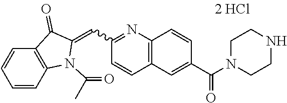 [SMILES: CC(=O)N1C(=Cc2ccc3cc(C(=O)N4CCNCC4)ccc3n2)C(=O)c2ccccc21] | Z/E Mix. (about 5:2) | | Production Ex. 202 | 1H NMR (270 MHz, dimethyl- sulfoxide-d6) δ 9.31 (s, 2H), 8.57-8.49 (m, 1H), 8.23-7.70 (m, 7H), 7.41- |
| | | | | | | 7.30 (m, 2H), |
| | | | | | | 3.57 (s, 8H), |
| | | | | | | 2.72, 2.09 (each |
| | | | | | | s, combined 2H). |
| | | | | | | MS 427.40 |
| | | | | | | (M + H). |
| 140 | (Z)-2-((4-([1,1′- biphenyl]-2-yl)- 6-(4-methylpiper- azine-1-carbonyl)- quinolin-2-yl)- methylene)-1- acetylindolin-3- one | 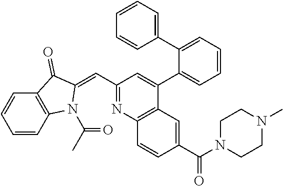 [SMILES: CC(=O)N1/C(=C\c2cc(-c3ccccc3-c3ccccc3)c3cc(C(=O)N4CCN(C)CC4)ccc3n2)C(=O)c2ccccc21] | Z only | Ex. 60 | Production Ex. 285 CAS No. 16800-68-3 (Reagent Supplier 3) | 1H NMR (500 MHz) δ 8.21- 8.16 (m, 1H), 8.06 (dd, J = 8.6, 0.6 Hz, 1H), 7.85-7.79 (m, 1H), 7.70-7.55 (m, 5H), 7.54- 7.49 (m, 1H), 7.41 (dd, J = 7.5, 1.3 Hz, 1H), 7.39 (s, 1H), |
| | | | | | | 7.26-7.21 (m, |
| | | | | | | 1H), 7.20 (s, |
| | | | | | | 1H), 7.07-6.98 |
| | | | | | | (m, 5H), 3.96- |
| | | | | | | 3.60 (m, 2H), |
| | | | | | | 3.38-3.18 (m, |
| | | | | | | 2H), 2.60-2.19 |
| | | | | | | (m, 7H), 2.02 (s, |
| | | | | | | 3H). MS 593.39 |
| | | | | | | (M + H). |
| 141 | (Z)-4-([1,1′- biphenyl]-2-yl)-2- ((1-acetyl-3-oxo- indolin-2-ylidene)- methyl)quinoline- 6-carboxamide | 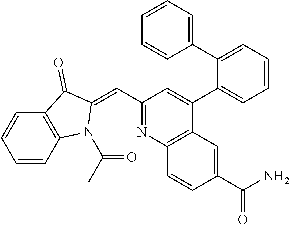 [SMILES: CC(=O)N1/C(=C\c2cc(-c3ccccc3-c3ccccc3)c3cc(C(N)=O)ccc3n2)C(=O)c2ccccc21] | Z only | Ex. 60 | Production Ex. 286 CAS No. 16800-68-3 (Reagent Supplier 3) | 1H NMR (500 MHz) δ 8.21- 8.16 (m, 1H), 8.12-8.07 (m, 1H), 8.06 (dd, J = 2.0, 0.6 Hz, 1H), 8.02-7.97 (m, 1H), 7.85- 7.80 (m, 1H), 7.69-7.58 (m, 3H), 7.58-7.52 (m, 1H), 7.48- 7.43 (m, 1H), 7.40 (s, 1H), |
| | | | | | | 7.25-7.21 (m, |
| | | | | | | 1H), 7.19 (s, |
| | | | | | | 1H), 7.08-6.99 |
| | | | | | | (m, 5H), 6.00 (s, |
| | | | | | | 1H), 5.69 (s, |
| | | | | | | 1H), 2.04 (s, |
| | | | | | | 3H). MS 510.42 |
| | | | | | | (M + H). |
| 142 | (Z)-1-(4-([1,1′- biphenyl]-2-yl)-2- ((1-acetyl-3-oxo- indolin-2-ylidene)- methyl)quinoline- 6-carbonyl)piper- idine-4-carboxylic acid | 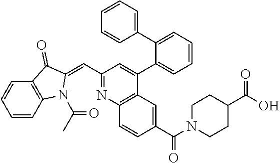 [SMILES: CC(=O)N1/C(=C\c2cc(-c3ccccc3-c3ccccc3)c3cc(C(=O)N4CCC(C(=O)O)CC4)ccc3n2)C(=O)c2ccccc21] | Z only | Ex. 127 | Production Ex. 385 | 1H NMR (500 MHz) δ 8.19- 8.04 (m, 2H), 7.88-7.79 (m, 1H), 7.74-7.47 (m, 6H), 7.46- 7.36 (m, 2H), 7.31-7.17 (m, 2H), 7.11-6.97 (m, 5H), 4.63- |
| | | | | | | 4.32 (m, 1H), |
| | | | | | | 3.75-3.36 (m, |
| | | | | | | 1H), 3.25-2.90 |
| | | | | | | (m, 2H), 2.68- |
| | | | | | | 2.59 (m, 1H), |
| | | | | | | 2.27-1.51 (m, |
| | | | | | | 7H). MS 622.47 |
| | | | | | | (M + H). |
| 143 | (Z)-2-((4-([1,1′- biphenyl]-2-yl)-6- (((tetrahydro-2H- pyran-4-yl)amino)- methyl)quinolin- 2-yl)methylene)- 1-acetylindolin- 3-one | 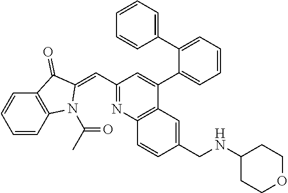 [SMILES: CC(=O)N1/C(=C\c2cc(-c3ccccc3-c3ccccc3)c3cc(CNC4CCOCC4)ccc3n2)C(=O)c2ccccc21] | Z only | Ex. 130 | Production Ex. 386 | 1H NMR (500 MHz) δ 8.21- 8.18 (m, 1H), 8.01 (d, J = 8.6 Hz, 1H), 7.82- 7.78 (m, 1H), 7.67-7.55 (m, 4H), 7.53-7.48 (m, 2H), 7.44- 7.40 (m, 1H), 7.28 (s, 1H), 7.23-7.19 (m, |
| | | | | | | 1H), 7.18 (s, |
| | | | | | | 1H), 7.05-6.97 |
| | | | | | | (m, 5H), 4.00- |
| | | | | | | 3.92 (m, 2H), |
| | | | | | | 3.85 (s, 2H), |
| | | | | | | 3.41-3.31 (m, |
| | | | | | | 2H), 2.73-2.64 |
| | | | | | | (m, 1H), 1.98 (s, |
| | | | | | | 3H), 1.85-1.77 |
| | | | | | | (m, 2H), 1.46- |
| | | | | | | 1.36 (m, 2H). |
| | | | | | | MS 580.49 |
| | | | | | | (M + H). |
| 144 | (Z)-1-acetyl-2- ((4-(2-acetyl- 1,2,3,4-tetra- hydroisoquinolin- 6-yl)-6-(morph- oline-4-carbonyl)- quinolin-2-yl)- methylene)indolin- 3-one | 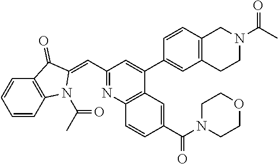 [SMILES: CC(=O)N1CCc2cc(-c3cc(/C=C4/C(=O)c5ccccc5N4C(C)=O)nc4ccc(C(=O)N5CCOCC5)cc34)ccc2C1] | Z only | Ex. 60 | Production Ex. 287 CAS No. 16800-68-3 (Reagent Supplier 3) | 1H NMR (500 MHz) δ 8.22- 8.17 (m, 2H), 7.96 (dd, J = 6.2, 1.8 Hz, 1H), 7.84 (dd, J = 7.7, 1.4 Hz, 1H), 7.74 (dd, J = 8.6, 1.8 Hz, 1H), 7.70- 7.64 (m, 1H), 7.55 (s, 1H), |
| | | | | | | 7.38-7.23 (m, |
| | | | | | | 5H), 4.86, 4.75 |
| | | | | | | (each s, com- |
| | | | | | | bined 2H), 3.95- |
| | | | | | | 3.35 (m, 10H), |
| | | | | | | 3.02, 2.95 (each |
| | | | | | | t, each J = 5.9 |
| | | | | | | Hz, combined |
| | | | | | | 2H), 2.26, 2.23 |
| | | | | | | (each s, com- |
| | | | | | | bined 3H), 2.19 |
| | | | | | | (s, 3H). MS |
| | | | | | | 601.52 (M + H). |
| 145 | 2-((4-(1H-indazol- 4-yl)-6-(morph- oline-4-carbonyl)- quinolin-2-yl)- methylene)-1- acetylindolin-3- one | 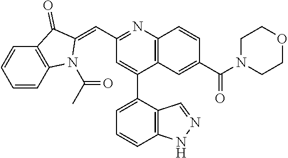 [SMILES: CC(=O)N1/C(=C\c2cc(-c3cccc4[nH]ncc34)c3cc(C(=O)N4CCOCC4)ccc3n2)C(=O)c2ccccc21] | Z/E Mix. (ca. 10:1) | Ex. 129 | Production Ex. 373 | 1H NMR (500 MHz) δ 10.46 (s, 0.3H), 8.24 (dd, J = 8.6, 0.7 Hz, 1H), 8.19- 8.16 (m, 1H), 7.86-7.82 (m, 1H), 7.80-7.79 (m, 1H), 7.77 (dd, J = 8.6, 1.8 |
| | | | | | | Hz, 1H), 7.73 |
| | | | | | | (d, J = 1.1 Hz, |
| | | | | | | 1H), 7.71 (s, |
| | | | | | | 1H), 7.68-7.64 |
| | | | | | | (m, 2H), 7.61- |
| | | | | | | 7.51 (m, 1H), |
| | | | | | | 7.36 (s, 1H), |
| | | | | | | 7.28-7.23 (m, |
| | | | | | | 2H), 3.82-3.32 |
| | | | | | | (m, 8H), 2.72, |
| | | | | | | 2.25 (each s, |
| | | | | | | combined 3H). |
| | | | | | | MS 544.38 |
| | | | | | | (M + H). |
| 146 | (Z)-1-acetyl-2- ((4-(2-acetyl- 1,2,3,4-tetra- hydroisoquin- olin-5-yl)-6- (morpholine-4- carbonyl)quinolin- 2-yl)methylene)- indolin-3-one | 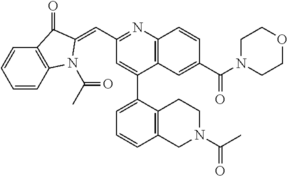 [SMILES: CC(=O)N1CCc2c(cccc2-c2cc(/C=C3/C(=O)c4ccccc4N3C(C)=O)nc3ccc(C(=O)N4CCOCC4)cc23)C1] | Z only | Ex. 60 | Production Ex. 291 CAS No. 16800-68-3 (Reagent Supplier 3) | 1H NMR (500 MHz) δ 8.22- 8.19 (m, 1H), 8.19-8.16 (m, 1H), 7.87-7.83 (m, 1H), 7.76- 7.71 (m, 1H), 7.70-7.64 (m, 1H), 7.56-7.52 (m, 1H), 7.50- 7.46 (m, 1H), |
| | | | | | | 7.42-7.23 (m, |
| | | | | | | 4H), 7.19-7.13 |
| | | | | | | (m, 1H), 4.88- |
| | | | | | | 4.74 (m, 2H), |
| | | | | | | 3.88-3.30 (m, |
| | | | | | | 10H), 2.62-2.27 |
| | | | | | | (m, 2H), 2.23 (s, |
| | | | | | | 3H), 2.23, 2.12 |
| | | | | | | (each s, com- |
| | | | | | | bined 3H). MS |
| | | | | | | 601.50 (M + H). |
| 147 | 6-(2-((1-acetyl-3- oxoindolin-2- ylidene)methyl)- 6-(morpholine-4- carbonyl)quinolin- 4-yl)-3,4-dihydro- isoquinolin-1(2H)- one | 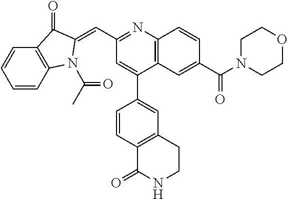 [SMILES: CC(=O)N1/C(=C\c2cc(-c3ccc4c(c3)CCNC4=O)c3cc(C(=O)N4CCOCC4)ccc3n2)C(=O)c2ccccc21] | Z/E Mix. (ca. 10:1) | Ex. 60 | Production Ex. 438 CAS No. 16800-68-3 (Reagent Supplier 3) | 1H NMR (500 MHz) δ 8.26- 8.23 (m, 1H), 8.21-8.15 (m, 2H), 7.89 (d, J = 1.8 Hz, 1H), 7.85-7.81 (m, 1H), 7.74 (dd, J = 8.6, 1.8 Hz, 1H), 7.68-7.63 (m, 1H), 7.56 (s, 1H), 7.47 (dd, J = 7.9, 1.7 Hz, |
| | | | | | | 1H), 7.36 (d, J = |
| | | | | | | 1.7 Hz, 1H), |
| | | | | | | 7.33 (s, 1H), |
| | | | | | | 7.28-7.22 (m, |
| | | | | | | 1H), 6.18 (s, |
| | | | | | | 1H), 3.77 (s, |
| | | | | | | 10H), 3.11 (t, |
| | | | | | | J = 6.5 Hz, 2H), |
| | | | | | | 2.72, 2.19 (each |
| | | | | | | s, combined |
| | | | | | | 3H). MS 573.32 |
| | | | | | | (M + H). |
| 148 | (Z)-2-((4-(3-(1H- pyrazol-4-yl)phen- yl)-6-(morpholine- 4-carbonyl)quin- olin-2-yl)methyl- ene)-1-acetylin- dolin-3-one | 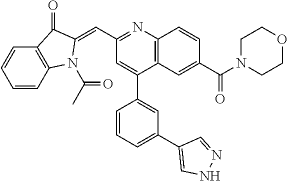 [SMILES: CC(=O)N1/C(=C\c2cc(-c3cccc(-c4cn[nH]c4)c3)c3cc(C(=O)N4CCOCC4)ccc3n2)C(=O)c2ccccc21] | Z only | Ex. 129 | Production Ex. 390 | 1H NMR (500 MHz) δ 8.20 (t, J = 8.2 Hz, 2H), 8.00 (d, J = 1.8 Hz, 1H), 7.93 (s, 2H), 7.85 (dd, J = 7.6, 1.4 Hz, 1H), 7.76 (dd, J = 8.6, 1.9 Hz, 1H), 7.70- 7.65 (m, 2H), 7.64-7.61 (m, |
| | | | | | | 2H), 7.57 (t, J = |
| | | | | | | 7.7 Hz, 1H), |
| | | | | | | 7.39-7.35 (m, |
| | | | | | | 2H), 7.26 (t, J = |
| | | | | | | 7.4 Hz, 1H), |
| | | | | | | 3.86-3.35 (m, |
| | | | | | | 8H), 2.21 (s, 3H). |
| | | | | | | MS 570.34 |
| | | | | | | (M + H). |
| 149 | (Z)-1-acetyl-2- ((4-(3-(1-methyl- 1H-pyrazol-4-yl)- phenyl)-6-(morph- oline-4-carbonyl)- quinolin-2-yl)- methylene)indolin- 3-one | 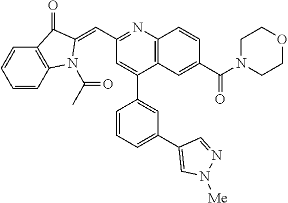 [SMILES: CC(=O)N1/C(=C\c2cc(-c3cccc(-c4cnn(C)c4)c3)c3cc(C(=O)N4CCOCC4)ccc3n2)C(=O)c2ccccc21] | Z only | Ex. 2 | Production Ex. 294 CAS No. 16800-68-3 (Reagent Supplier 3) | 1H NMR (500 MHz) δ 8.21- 8.17 (m, 2H), 7.98-7.96 (m, 1H), 7.85-7.82 (m, 1H), 7.80 (d, J = 0.8 Hz, 1H), 7.74 (dd, J = 8.7, 1.8 Hz, 1H), 7.70-7.61 (m, 3H), 7.60 (s, 1H), 7.56 (t, J = 1.8 Hz, 1H), 7.53 (t, J = |
| | | | | | | 7.7 Hz, 1H), 7.35 |
| | | | | | | (s, 1H), 7.34- |
| | | | | | | 7.31 (m, 1H), |
| | | | | | | 7.27-7.22 (m, |
| | | | | | | 1H), 3.96 (s, 3H), |
| | | | | | | 3.88-3.31 (m, |
| | | | | | | 8H), 2.19 (s, 3H). |
| | | | | | | MS 584.37 |
| | | | | | | (M + H). |
| 150 | 2-((4-([1,1′-biphenyl]-4-yl)-6- (morpholine-4- carbonyl)quinolin- 2-yl)methylene)- 1-acetylindolin-3- one | 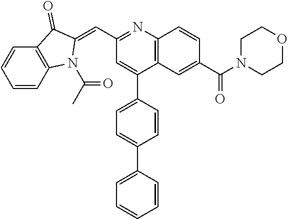 [SMILES: CC(=O)N1/C(=C\c2cc(-c3ccc(-c4ccccc4)cc3)c3cc(C(=O)N4CCOCC4)ccc3n2)C(=O)c2ccccc21] | | | | |
| 151 | 1-acetyl-2-((6- (morpholine-4- carbonyl)-4- (naphthalen-2- yl)quinolin-2- yl)methylene)- indolin-3-one | 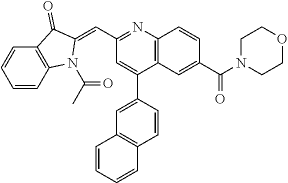 [SMILES: CC(=O)N1/C(=C\c2cc(-c3ccc4ccccc4c3)c3cc(C(=O)N4CCOCC4)ccc3n2)C(=O)c2ccccc21] | | | | |
| 152 | (Z)-4-([1,1′- biphenyl]-3-yl)-2- ((1-acetyl-3-oxo- indolin-2-ylidene)- methyl)quinoline- 6-carboxylic acid | 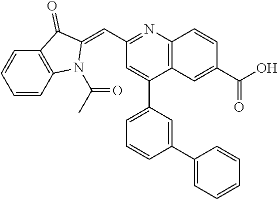 [SMILES: CC(=O)N1/C(=C\c2cc(-c3cccc(-c4ccccc4)c3)c3cc(C(=O)O)ccc3n2)C(=O)c2ccccc21] | Z only | Ex. 127 | Production Ex. 374 | 1H NMR (400 MHz) δ 8.80 (d, J = 1.8 Hz, 1H), 8.36 (dd, J = 8.8, 1.9 Hz, 1H), 8.28-8.17 (m, 2H), 7.85 (dd, J = 7.9, 1.5 Hz, 1H), 7.82-7.77 (m, 1H), 7.77- 7.74 (m, 1H), 7.71-7.63 (m, |
| | | | | | | 5H), 7.55-7.45 |
| | | | | | | (m, 3H), 7.42- |
| | | | | | | 7.35 (m, 2H), |
| | | | | | | 7.30-7.21 (m, |
| | | | | | | 1H), 2.23 (s, |
| | | | | | | 3H). MS 511.13 |
| | | | | | | (M + H). |
| 153 | (Z)-2-((1-acetyl- 3-oxoindolin-2- ylidene)methyl)- 4-(naphthalen-1- yl)quinoline-6- carboxylic acid | 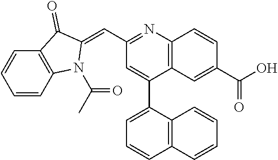 [SMILES: CC(=O)N1/C(=C\c2cc(-c3cccc4ccccc34)c3cc(C(=O)O)ccc3n2)C(=O)c2ccccc21] | Z only | Ex. 127 | Production Ex. 375 | 1H NMR (400 MHz) δ 8.33- 8.16 (m, 4H), 8.11-7.93 (m, 2H), 7.85 (dd, J = 7.6, 1.4 Hz, 1H), 7.76-7.60 (m, 3H), 7.56- 7.46 (m, 2H), 7.40-7.30 (m, |
| | | | | | | 3H), 7.29-7.21 |
| | | | | | | (m, 1H), 2.29 (s, |
| | | | | | | 3H). MS 485.16 |
| | | | | | | (M + H). |
| 154 | 2-((1-acetyl-3- oxoindolin-2- ylidene)methyl)- [4,4′-biquinoline]- 6-carboxylic acid | 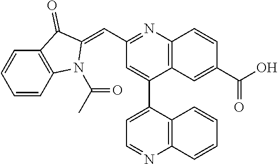 [SMILES: CC(=O)N1/C(=C\c2cc(-c3ccnc4ccccc34)c3cc(C(=O)O)ccc3n2)C(=O)c2ccccc21] | | | | |
| 155 | (Z)-2-((1-acetyl- 3-oxoindolin-2- ylidene)methyl)- [4,8′-biquinoline]- 6-carboxylic acid | 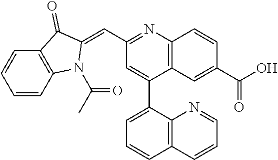 [SMILES: CC(=O)N1/C(=C\c2cc(-c3cccc4cccnc34)c3cc(C(=O)O)ccc3n2)C(=O)c2ccccc21] | Z only | Ex. 127 | Production Ex. 377 | 1H NMR (400 MHz, dimethyl- sulfoxide-d6) δ 8.80-8.74 (m, 1H), 8.56 (dd, J = 8.5, 1.8 Hz, 1H), 8.29-8.20 (m, 2H), 8.09 (s, 1H), 8.07 (s, 1H), 8.03 (s, 1H), |
| | | | | | | 7.94-7.75 (m, |
| | | | | | | 5H), 7.62 (dd, |
| | | | | | | J = 8.4, 4.4 Hz, |
| | | | | | | 1H), 7.43 (s, |
| | | | | | | 1H), 7.34 (t, J = |
| | | | | | | 7.4 Hz, 1H), |
| | | | | | | 2.23 (s, 3H). MS |
| | | | | | | 486.17 (M + H). |
| 156 | (Z)-4-([1,1′- biphenyl]-4-yl)- 2-((1-acetyl-3- oxoindolin-2- ylidene)methyl)- quinoline-6- carboxylic acid | 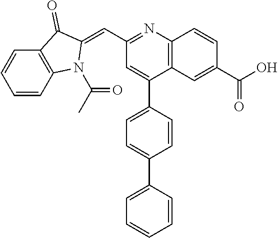 [SMILES: CC(=O)N1/C(=C\c2cc(-c3ccc(-c4ccccc4)cc3)c3cc(C(=O)O)ccc3n2)C(=O)c2ccccc21] | Z only | Ex. 127 | Production Ex. 376 | 1H NMR (400 MHz, dimethyl- sulfoxide-d6) δ 8.58 (d, J = 1.9 Hz, 1H), 8.33- 8.29 (m, 1H), 8.10-8.05 (m, 2H), 8.05 (s, 1H), 7.99-7.94 (m, 2H), 7.84 (d, J = 7.6 Hz, 6H), 7.57-7.51 (m, 2H), 7.47-7.41 (m, 2H), 7.34 (t, J = 7.4 Hz, 1H), |
| | | | | | | 2.17 (s, 3H). MS |
| | | | | | | 511.16 (M + H). |
| 157 | (Z)-2-((1-acetyl- 3-oxoindolin-2- ylidene)methyl)- 4-(naphthalen-2- yl)quinoline-6- carboxylic acid | 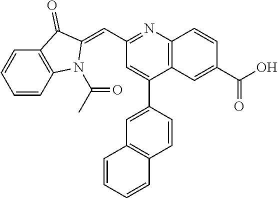 [SMILES: CC(=O)N1/C(=C\c2cc(-c3ccc4ccccc4c3)c3cc(C(=O)O)ccc3n2)C(=O)c2ccccc21] | Z only | Ex. 127 | Production Ex. 378 | 1H NMR (400 MHz, dimethyl- sulfoxide-d6) δ 8.54 (d, J = 1.9 Hz, 1H), 8.32 (dd, J = 8.7, 1.8 Hz, 1H), 8.22 (d, J = 1.7 Hz, 1H), 8.19 (d, J = 8.6 Hz, 1H), 8.12- 8.05 (m, 5H), 7.86-7.75 (m, |
| | | | | | | 3H), 7.70-7.63 |
| | | | | | | (m, 2H), 7.47 (s, |
| | | | | | | 1H), 7.34 (t, J = |
| | | | | | | 7.4 Hz, 1H), 2.18 |
| | | | | | | (s, 3H). MS |
| | | | | | | 485.19 (M + H). |
| 158 | (Z)-2-((4-([1,1′- biphenyl]-3-yl)-6- (4-(oxetan-3-yl)- piperazine-1- carbonyl)quinolin- 2-yl)methylene)- 1-acetylindolin-3- one | 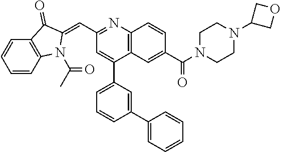 [SMILES: CC(=O)N1/C(=C\c2cc(-c3cccc(-c4ccccc4)c3)c3cc(C(=O)N4CCN(C5COC5)CC4)ccc3n2)C(=O)c2ccccc21] | Z only | Ex. 60 | Production Ex. 230 CAS No. 16800-68-3 (Reagent Supplier 3) | 1H NMR (500 MHz) δ 8.23- 8.17 (m, 2H), 7.99 (dd, J = 1.9, 0.6 Hz, 1H), 7.86-7.82 (m, 1H), 7.80-7.74 (m, 2H), 7.71 (t, J = 1.8 Hz, 1H), 7.69-7.61 (m, |
| | | | | | | 5H), 7.51-7.45 |
| | | | | | | (m, 3H), 7.42- |
| | | | | | | 7.38 (m, 1H), |
| | | | | | | 7.36 (s, 1H), |
| | | | | | | 7.27-7.23 (m, |
| | | | | | | 1H), 4.63 (t, J = |
| | | | | | | 6.6 Hz, 2H), 4.55 |
| | | | | | | (t, J = 6.2 Hz, |
| | | | | | | 2H), 3.89-3.72 |
| | | | | | | (m, 2H), 3.54 |
| | | | | | | 3.35 (m, 3H), |
| | | | | | | 2.48-2.03 (m, |
| | | | | | | 7H). MS 635.36 |
| | | | | | | (M + H). |
| 159 | (Z)-1-acetyl-2-((4- (naphthalen-1-yl)- 6-(4-(oxetan-3- yl)piperazine-1- carbonyl)quinolin- 2-yl)methylene)- indolin-3-one | 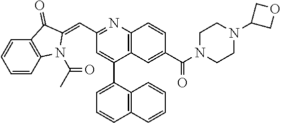 [SMILES: CC(=O)N1/C(=C\c2cc(-c3cccc4ccccc34)c3cc(C(=O)N4CCN(C5COC5)CC4)ccc3n2)C(=O)c2ccccc21] | Z only | Ex. 2 | Production Ex. 229 CAS No. 16800-68-3 (Reagent Supplier 3) | 1H NMR (400 MHz) δ 8.24 (d, J = 8.8 Hz, 1H), 8.19 (d, J = 8.3 Hz, 1H), 8.04 (d, J = 8.3 Hz, 1H), 7.99 (d, J = 8.2 Hz, 1H), 7.85 |
| | | | | | | (dd, J = 7.9, 1.4 |
| | | | | | | Hz, 1H), 7.77 |
| | | | | | | (dd, J = 8.7, 1.8 |
| | | | | | | Hz, 1H), 7.70- |
| | | | | | | 7.61 (m, 3H), |
| | | | | | | 7.55-7.46 (m, |
| | | | | | | 2H), 7.40-7.22 |
| | | | | | | (m, 5H), 4.66- |
| | | | | | | 4.59 (m, 2H), |
| | | | | | | 4.57-4.49 (m, |
| | | | | | | 2H), 3.87-3.14 |
| | | | | | | (m, 5H), 2.41- |
| | | | | | | 1.63 (m, 7H). |
| | | | | | | MS 609.25 |
| | | | | | | (M + H). |
| 160 | 1-acetyl-2-((6-(4- (oxetan-3-yl)- piperazine-1- carbonyl)-[4,4′- biquinolin]-2- yl)methylene)- indolin-3-one | 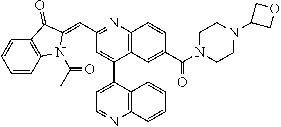 [SMILES: CC(=O)N1/C(=C\c2cc(-c3ccnc4ccccc34)c3cc(C(=O)N4CCN(C5COC5)CC4)ccc3n2)C(=O)c2ccccc21] | | | | |
| 161 | 1-acetyl-2-((6-(4- (oxetan-3-yl)- piperazine-1- carbonyl)-[4,8′- biquinolin]-2- yl)methylene)- indolin-3-one | 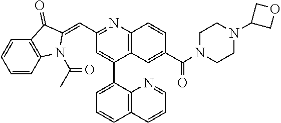 [SMILES: CC(=O)N1/C(=C\c2cc(-c3cccc4cccnc34)c3cc(C(=O)N4CCN(C5COC5)CC4)ccc3n2)C(=O)c2ccccc21] | | | | |
| 162 | (Z)-2-((4-([1,1′- biphenyl]-4-yl)-6- (4-(oxetan-3-yl)- piperazine-1-car- bonyl)quinolin-2- yl)methylene)-1- acetylindolin-3- one | 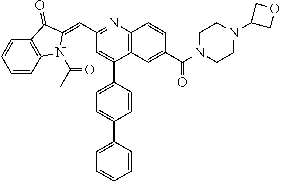 [SMILES: CC(=O)N1/C(=C\c2cc(-c3ccc(-c4ccccc4)cc3)c3cc(C(=O)N4CCN(C5COC5)CC4)ccc3n2)C(=O)c2ccccc21] | Z only | Ex. 60 | Production Ex. 231 CAS No. 16800-68-3 (Reagent Supplier 3) | 1H NMR (500 MHz) δ 8.23- 8.17 (m, 2H), 8.05 d, J = 1.7 Hz, 1H), 7.87- 7.82 (m, 1H), 7.81-7.77 (m, 2H), 7.75 (dd, J = 8.7, 1.8 Hz, 1H), 7.71-7.64 (m, 3H), 7.62 (s, |
| | | | | | | 1H), 7.61-7.58 |
| | | | | | | (m, 2H), 7.54- |
| | | | | | | 7.49 (m, 2H), |
| | | | | | | 7.45-7.38 (m, |
| | | | | | | 1H), 7.37 (s, |
| | | | | | | 1H), 7.28-7.23 |
| | | | | | | (m, 1H), 4.65 (t, |
| | | | | | | J = 6.6 Hz, 2H), |
| | | | | | | 4.59 (t, J = 6.2 |
| | | | | | | Hz, 2H), 3.92- |
| | | | | | | 3.74 (m, 2H), |
| | | | | | | 3.57-3.41 (m, |
| | | | | | | 3H), 2.49-2.17 |
| | | | | | | (m, 7H). MS |
| | | | | | | 635.33 (M + H). |
| 163 | (Z)-1-acetyl-2- ((4-(naphthalen-2- yl)-6-(4-(oxetan-3- yl)piperazine-1- carbonyl)quinolin- 2-yl)methylene)- indolin-3-one | 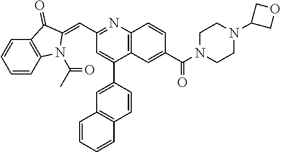 [SMILES: CC(=O)N1/C(=C\c2cc(-c3ccc4ccccc4c3)c3cc(C(=O)N4CCN(C5COC5)CC4)ccc3n2)C(=O)c2ccccc21] | Z only | Ex. 2 | Production Ex. 236 CAS No. 16800-68-3 (Reagent Supplier 3) | 1H NMR (400 MHz) δ 8.24- 8.18 (m, 2H), 8.03 (d, J = 8.4 Hz, 1H), 8.00- 7.90 (m, 4H), 7.85 (dd, J = 7.4, 1.4 Hz, 1H), 7.76 (dd, J = 8.7, 1.8 Hz, 1H), |
| | | | | | | 7.70-7.58 (m, |
| | | | | | | 5H), 7.38 (s, |
| | | | | | | 1H), 7.29-7.23 |
| | | | | | | (m, 1H), 4.63 (t, |
| | | | | | | J = 6.6 Hz, 2H), |
| | | | | | | 4.57 (t, J = 6.2 |
| | | | | | | Hz, 2H), 3.89- |
| | | | | | | 3.34 (m, 5H), |
| | | | | | | 2.49-2.09 (m, |
| | | | | | | 7H). MS 609.5 |
| | | | | | | (M + H). |
| 164 | (Z)-2-((4-([1,1′- biphenyl]-3-yl)-6- (((tetrahydro-2H- pyran-4-yl)amino)- methyl)quinolin- 2-yl)methylene)- 1-acetylindolin-3- one | 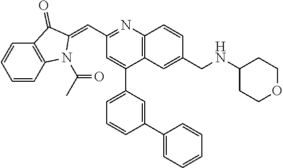 [SMILES: CC(=O)N1/C(=C\c2cc(-c3cccc(-c4ccccc4)c3)c3cc(CNC4CCOCC4)ccc3n2)C(=O)c2ccccc21] | Z only | Ex. 130 | Production Ex. 380 | 1H NMR (400 MHz) δ 8.22 (d, J = 8.3 Hz, 1H), 8.15 (d, J = 8.7 Hz, 1H), 7.87 (d, J = 1.9 Hz, 1H), 7.84 (dd, J = 7.7, 1.4 Hz, 1H), 7.78-7.73 (m, 3H), 7.69-7.61 (m, 4H), 7.59 |
| | | | | | | (s, 1H), 7.53- |
| | | | | | | 7.45 (m, 3H), |
| | | | | | | 7.42-7.38 (m, |
| | | | | | | 1H), 7.38 (s, |
| | | | | | | 1H), 7.25 (t, J = |
| | | | | | | 7.5 Hz, 1H), 3.96 |
| | | | | | | (s, 2H), 3.95-3.87 |
| | | | | | | (m, 2H), 3.37- |
| | | | | | | 3.27 (m, 2H), |
| | | | | | | 2.76-2.66 (m, |
| | | | | | | 1H), 2.20 (s, |
| | | | | | | 3H), 1.85-1.76 |
| | | | | | | (m, 2H), 1.47- |
| | | | | | | 1.34 (m, 2H). MS |
| | | | | | | 580.26 (M + H). |
| 165 | 1-acetyl-2-((4- (naphthalen-1-yl)- 6-(((tetrahydro- 2H-pyran-4-yl)- amino)methyl)- quinolin-2-yl)- methylene)indolin- 3-one | 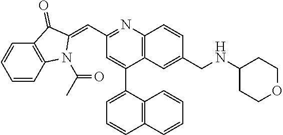 [SMILES: CC(=O)N1/C(=C\c2cc(-c3cccc4ccccc34)c3cc(CNC4CCOCC4)ccc3n2)C(=O)c2ccccc21] | | | | |
| 166 | 1-acetyl-2-((6- (((tetrahydro-2H- pyran-4-yl)amino)- methyl)-[4,4′- biquinolin]-2-yl)- methylene)indolin- 3-one | 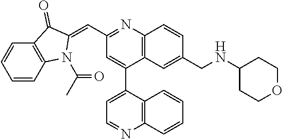 [SMILES: CC(=O)N1/C(=C\c2cc(-c3ccnc4ccccc34)c3cc(CNC4CCOCC4)ccc3n2)C(=O)c2ccccc21] | | | | |
| 167 | (Z)-1-acetyl-2- ((6-(((tetrahydro- 2H-pyran-4-yl)- amino)methyl)- [4,8′-biquinolin]- 2-yl)methylene)- indolin-3-one | 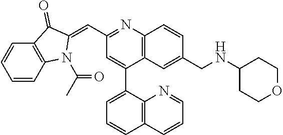 [SMILES: CC(=O)N1/C(=C\c2cc(-c3cccc4cccnc34)c3cc(CNC4CCOCC4)ccc3n2)C(=O)c2ccccc21] | Z only | Ex. 130 | Production Ex. 382 | 1H NMR (400 MHz) δ 8.82 (dd, J = 4.4, 1.8 Hz, 1H), 8.30 (dd, J = 8.4, 1.8 Hz, 1H), 8.23 (d, J = 8.4 Hz, 1H), 8.16 (d, J = 8.7 Hz, |
| | | | | | | 1H), 8.02 (dd, J = |
| | | | | | | 7.3, 2.4 Hz, 1H), |
| | | | | | | 7.83 (d, J = 7.4 |
| | | | | | | Hz, 1H), 7.76- |
| | | | | | | 7.61 (m, 5H), |
| | | | | | | 7.47 (dd, J = 8.3, |
| | | | | | | 4.2 Hz, 1H), 7.41 |
| | | | | | | (s, 1H), 7.31 (d, |
| | | | | | | J = 1.8 Hz, 1H), |
| | | | | | | 7.27-7.20 (m, |
| | | | | | | 1H), 3.94-3.83 |
| | | | | | | (m, 2H), 3.81 (s, |
| | | | | | | 2H), 3.34-3.20 |
| | | | | | | (m, 2H), 2.64- |
| | | | | | | 2.54 (m, 1H), |
| | | | | | | 2.29 (s, 3H), |
| | | | | | | 1.77-1.58 (m, |
| | | | | | | 2H), 1.38-1.23 |
| | | | | | | (m, 2H). MS |
| | | | | | | 555.21 (M + H). |
| 168 | (Z)-2-((4-([1,1′- biphenyl]-4-yl)-6- (((tetrahydro-2H- pyran-4-yl)amino)- methyl)quinolin- 2-yl)methylene)- 1-acetylindolin-3- one | 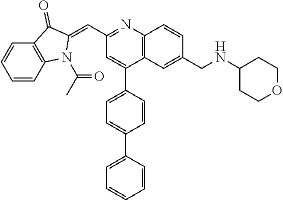 [SMILES: CC(=O)N1/C(=C\c2cc(-c3ccc(-c4ccccc4)cc3)c3cc(CNC4CCOCC4)ccc3n2)C(=O)c2ccccc21] | Z only | Ex. 130 | Production Ex. 379 | 1H NMR (400 MHz) δ 8.23 (d, J = 8.3 Hz, 1H), 8.15 (d, J = 8.7 Hz, 1H), 7.88- 7.75 (m, 5H), 7.73-7.63 (m, 3H), 7.63-7.59 (m, 2H), 7.57 (s, 1H), 7.54-7.49 (m, 2H), 7.45- 7.39 (m, 1H), |
| | | | | | | 7.38 (s, 1H), |
| | | | | | | 7.25 (t, J = 7.5 |
| | | | | | | Hz, 1H), 4.02- |
| | | | | | | 3.92 (m, 4H), |
| | | | | | | 3.42-3.33 (m, |
| | | | | | | 2H), 2.79-2.69 |
| | | | | | | (m, 1H), 2.20 (s, |
| | | | | | | 3H), 1.89-1.81 |
| | | | | | | (m, 2H), 1.50- |
| | | | | | | 1.38 (m, 2H). |
| | | | | | | MS 580.26 |
| | | | | | | (M + H). |
| 169 | (Z)-1-acetyl-2-((4- (naphthalen-2-yl)- 6-(((tetrahydro- 2H-pyran-4-yl)- amino)methyl)- quinolin-2-yl)- methylene)indolin- 3-one | 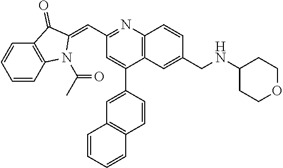 [SMILES: CC(=O)N1/C(=C\c2cc(-c3ccc4ccccc4c3)c3cc(CNC4CCOCC4)ccc3n2)C(=O)c2ccccc21] | Z only | Ex. 130 | Production Ex. 381 | 1H NMR (400 MHz) δ 8.23 (d, J = 8.2 Hz, 1H), 8.16 (d, J = 8.6 Hz, 1H), 8.05- 7.91 (m, 4H), 7.86-7.83 (m, 1H), 7.82 (d, J = 1.9 Hz, 1H), 7.78 (dd, J = 8.7, 1.9 Hz, 1H), 7.69- |
| | | | | | | 7.57 (m, 5H), |
| | | | | | | 7.39 (s, 1H), 7.25 |
| | | | | | | (t, J = 7.4 Hz, |
| | | | | | | 1H), 3.99-3.90 |
| | | | | | | (m, 4H), 3.40- |
| | | | | | | 3.30 (m, 2H), |
| | | | | | | 2.75-2.66 (m, |
| | | | | | | 1H), 2.21 (s, |
| | | | | | | 3H), 1.85-1.76 |
| | | | | | | (m, 2H), 1.47- |
| | | | | | | 1.34 (m, 2H). |
| | | | | | | MS 554.24 |
| | | | | | | (M + H). |
| 170 | (Z)-1-acetyl-2- ((6-(morpholine- 4-carbonyl)-4- (1H-pyrazol-4- yl)quinolin-2-yl)- methylene)indolin- 3-one | 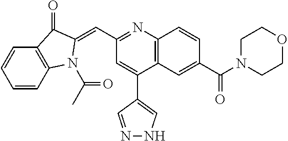 [SMILES: CC(=O)N1/C(=C\c2cc(-c3cn[nH]c3)c3cc(C(=O)N4CCOCC4)ccc3n2)C(=O)c2ccccc21] | Z only | Ex. 129 | Production Ex. 391 | 1H NMR (500 MHz) δ 8.26 (d, J = 1.8 Hz, 1H), 8.21-8.15 (m, 2H), 7.97 (s, 2H), 7.87-7.82 (m, 1H), 7.74 (dd, J = 8.6, 1.9 Hz, 1H), 7.70-7.64 (m, |
| | | | | | | 1H), 7.62 (s, 1H), |
| | | | | | | 7.34 (s, 1H), 7.26 |
| | | | | | | (t, J = 7.4 Hz, |
| | | | | | | 1H), 3.98-3.36 |
| | | | | | | (m, 8H), 2.17 (s, |
| | | | | | | 3H). MS 494.32 |
| | | | | | | (M + H). |
| 171 | (Z)-1-acetyl-2-((4- (1-methyl-1H- pyrazol-4-yl)-6- (morpholine-4- carbonyl)quinolin- 2-yl)methylene)- indolin-3-one | 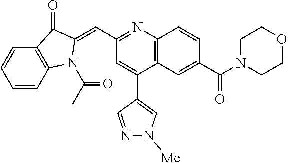 [SMILES: CC(=O)N1/C(=C\c2cc(-c3cnn(C)c3)c3cc(C(=O)N4CCOCC4)ccc3n2)C(=O)c2ccccc21] | Z only | Ex. 60 | Production Ex. 297 CAS No. 16800-68-3 (Reagent Supplier 3) | 1H NMR (500 MHz) δ 8.24 (d, J = 1.7 Hz, 1H), 8.21-8.18 (m, 1H), 8.16 (dd, J = 8.6, 0.6 Hz, 1H), 7.86-7.83 (m, 1H), 7.83 (d, J = 0.8 Hz, 1H), 7.78 (s, 1H), 7.73 |
| | | | | | | (dd, J = 8.6, 1.8 |
| | | | | | | Hz, 1H), 7.69- |
| | | | | | | 7.64 (m, 1H), |
| | | | | | | 7.58 (s, 1H), |
| | | | | | | 7.32 (s, 1H), |
| | | | | | | 7.28-7.23 (m, |
| | | | | | | 1H), 4.07 (s, |
| | | | | | | 3H), 3.96-3.36 |
| | | | | | | (m, 8H), 2.16 (s, |
| | | | | | | 3H). MS 508.26 |
| | | | | | | (M + H). |
| 172 | (Z)-1-acetyl-2-((6- (morpholine-4- carbonyl)-3-(naph- thalen-1-yl)quin- olin-2-yl)meth- ylene)indolin-3- one | 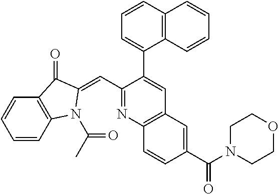 [SMILES: CC(=O)N1/C(=C\c2nc3ccc(C(=O)N4CCOCC4)cc3cc2-c2cccc3ccccc23)C(=O)c2ccccc21] | Z only | Ex. 60 | Production Ex. 292 CAS No. 16800-68-3 (Reagent Supplier 3) | 1H NMR (500 MHz) δ 8.25- 8.21 (m, 1H), 8.20 (d, J = 0.9 Hz, 1H), 8.13- 8.10 (m, 1H), 8.02 (dd, J = 8.3, 1.2 Hz, 1H), 8.01-7.98 (m, 1H), 7.92 (d, J = 1.8 Hz, 1H), 7.78 (dd, J = 8.7, 1.9 |
| | | | | | | Hz, 1H), 7.71- |
| | | | | | | 7.68 (m, 1H), |
| | | | | | | 7.64-7.59 (m, |
| | | | | | | 2H), 7.56-7.52 |
| | | | | | | (m, 1H), 7.46 |
| | | | | | | (dd, J = 7.0, 1.2 |
| | | | | | | Hz, 1H), 7.43- |
| | | | | | | 7.38 (m, 2H), |
| | | | | | | 7.20-7.15 (m, |
| | | | | | | 1H), 6.91 (s, |
| | | | | | | 1H), 3.98-3.42 |
| | | | | | | (m, 8H), 2.22 (s, |
| | | | | | | 3H). MS 554.5 |
| | | | | | | (M + H). |
| 173 | (Z)-1-acetyl-2- ((4-(2-methyl- 1,2,3,4-tetrahydro- isoquinolin-6-yl)- 6-(morpholine- 4-carbonyl)quin- olin-2-yl)meth- ylene)indolin-3- one | 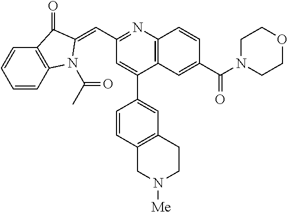 [SMILES: CC(=O)N1/C(=C\c2cc(-c3ccc4c(c3)CCN(C)C4)c3cc(C(=O)N4CCOCC4)ccc3n2)C(=O)c2ccccc21] | Z only | Ex. 60 | Production Ex. 350 CAS No. 16800-68-3 (Reagent Supplier 3) | 1H NMR (500 MHz) δ 8.22- 8.17 (m, 2H), 7.95 (d, J = 1.8 Hz, 1H), 7.86- 7.82 (m, 1H), 7.74 (dd, J = 8.7, 1.8 Hz, 1H), 7.69-7.64 (m, 1H), 7.55 (s, 1H), 7.34 (s, 1H), 7.30-7.21 (m, 4H), 3.91- |
| | | | | | | 3.34 (m, 10H), |
| | | | | | | 3.09 (t, J = 5.6 |
| | | | | | | Hz, 2H), 2.89 |
| | | | | | | (t, J = 6.0 Hz, |
| | | | | | | 2H), 2.60 (s, |
| | | | | | | 3H), 2.19 (s, |
| | | | | | | 3H). MS 573.36 |
| | | | | | | (M + H). |
| 174 | (Z)-4-(2-((1-acetyl- 3-oxoindolin-2- ylidene)methyl)-6- (morpholine-4- carbonyl)quinolin- 4-yl)benzoic acid | 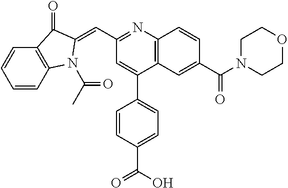 [SMILES: CC(=O)N1/C(=C\c2cc(-c3ccc(C(=O)O)cc3)c3cc(C(=O)N4CCOCC4)ccc3n2)C(=O)c2ccccc21] | Z only | Ex. 127 | Production Ex. 387 | 1H NMR (500 MHz) δ 8.29- 8.24 (m, 3H) 8.17-8.14 (m, 1H), 8.01 (d, J = 1.7 Hz, 1H), 7.87-7.83 (m, 1H), 7.78 (dd, J = 8.7, 1.8 Hz, 1H), 7.70-7.63 (m, 3H), 7.62 (s, 1H), 7.37 (s, |
| | | | | | | 1H), 7.29-7.25 |
| | | | | | | (m, 1H), 3.93- |
| | | | | | | 3.39 (m, 8H), |
| | | | | | | 2.24 (s, 3H). |
| | | | | | | MS 548.32 |
| | | | | | | (M + H). |
| 175 | (Z)-3-(2-((1-acetyl- 3-oxoindolin-2- ylidene)methyl)- 6-(morpholine-4- carbonyl)quinolin- 4-yl)benzoic acid | 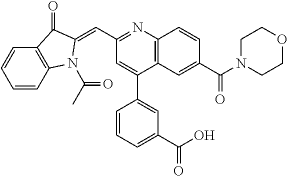 [SMILES: CC(=O)N1/C(=C\c2cc(-c3cccc(C(=O)O)c3)c3cc(C(=O)N4CCOCC4)ccc3n2)C(=O)c2ccccc21] | Z only | Ex. 127 | Production Ex. 388 | 1H NMR (500 MHz) δ 8.29- 8.21 (m, 3H), 8.18-8.13 (m, 1H), 7.91 (d, J = 1.8 Hz, 1H), 7.87-7.83 (m, 1H), 7.81-7.74 (m, 2H), 7.72- 7.64 (m, 2H), 7.61 (s, 1H), |
| | | | | | | 7.37 (s, 1H), |
| | | | | | | 7.29-7.24 (m, |
| | | | | | | 1H), 3.89-3.40 |
| | | | | | | (m, 8H), 2.23 (s, |
| | | | | | | 3H). MS 548.36 |
| | | | | | | (M + H). |
| 176 | (Z)-2-(2-((1-acetyl- 3-oxoindolin-2- ylidene)methyl)- 6-(morpholine-4- carbonyl)quinolin- 4-yl)benzoic acid | 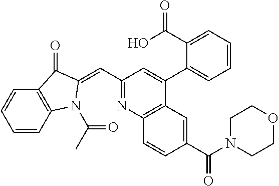 [SMILES: CC(=O)N1/C(=C\c2cc(-c3ccccc3C(=O)O)c3cc(C(=O)N4CCOCC4)ccc3n2)C(=O)c2ccccc21] | Z only | Ex. 127 | Production Ex. 389 | 1H NMR (500 MHz) δ 8.19 (dd, J = 8.7, 0.7 Hz, 1H), 8.17-8.14 (m, 1H), 8.11- 8.07 (m, 1H), 7.85-7.82 (m, 1H), 7.72-7.60 (m, 4H), 7.49 (s, 1H), 7.44 (dd, J = 1.9, 0.6 Hz, 1H), 7.34-7.30 |
| | | | | | | (m, 2H), 7.29- |
| | | | | | | 7.25 (m, 1H), |
| | | | | | | 3.79-3.14 (m, |
| | | | | | | 8H), 2.17 (s, 3H). |
| | | | | | | MS 548.29 |
| | | | | | | (M + H). |
| 177 | (Z)-1-acetyl-2-((4- (1-methyl-1H- pyrazol-4-yl)quin- olin-2-yl)meth- ylene)-indolin-3- one | 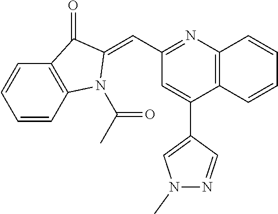 [SMILES: CC(=O)N1/C(=C\c2cc(-c3cnn(C)c3)c3ccccc3n2)C(=O)c2ccccc21] | Z only | | Production Ex. 302 CAS No. 16800-68-3 (Reagent Supplier 3) | 1H NMR (500 MHz) δ 8.22 (d, J = 8.3 Hz, 1H), 8.17 (d, J = 8.3 Hz, 1H), 8.14 (d, J = 8.4 Hz, 1H), 7.89-7.81 (m, 2H), 7.78-7.72 (m, 2H), 7.69- 7.63 (m, 1H), 7.60-7.56 (m, 1H), 7.54 (s, 1H), 7.34 (s, |
| | | | | | | 1H), 7.24 (t, J = |
| | | | | | | 7.3 Hz, 1H), |
| | | | | | | 4.07 (s, 3H), |
| | | | | | | 2.16 (s, 3H). |
| | | | | | | MS 395.29 |
| | | | | | | (M + H). |
| 178 | (Z)-1-acetyl-2-((4- (1-ethyl-1H-pyr- azol-4-yl)-6- (morpholine-4- carbonyl)quinolin- 2-yl)methylene)- indolin-3-one | 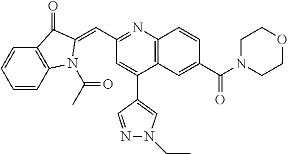 [SMILES: CCn1cc(-c2cc(/C=C3/C(=O)c4ccccc4N3C(C)=O)nc3ccc(C(=O)N4CCOCC4)cc23)cn1] | Z only | Ex. 2 | Production Ex. 304 CAS No. 16800-68-3 (Reagent Supplier 3) | 1H NMR (500 MHz) δ 8.25 (d, J = 1.8 Hz, 1H), 8.19 (d, J = 8.6 Hz, 1H), 8.16 (d, J = 8.6 Hz, 1H), 7.86-7.80 (m, 3H), 7.73 (dd, J = 8.6, 1.8 Hz, 1H), 7.68-7.64 |
| | | | | | | (m, 1H), 7.59 |
| | | | | | | (s, 1H), 7.32 (s, |
| | | | | | | 1H), 7.27-7.23 |
| | | | | | | (m, 1H), 4.33 (q, |
| | | | | | | J = 7.4 Hz, 2H), |
| | | | | | | 3.95-3.38 (m, |
| | | | | | | 8H), 2.16 (s, |
| | | | | | | 3H), 1.62 (t, J = |
| | | | | | | 7.4 Hz, 3H). MS |
| | | | | | | 522.36 (M + H). |
| 179 | (Z)-1-acetyl-2- ((4-(1-acetylpiper- idin-3-yl)-6- (morpholine-4- carbonyl)quinolin- 2-yl)methylene)- indolin-3-one | 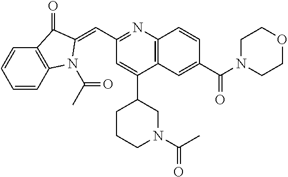 [SMILES: CC(=O)N1CCCC(c2cc(/C=C3/C(=O)c4ccccc4N3C(C)=O)nc3ccc(C(=O)N4CCOCC4)cc23)C1] | Z only | Ex. 2 | Production Ex. 306 CAS No. 16800-68-3 (Reagent Supplier 3) | 1H NMR (500 MHz) δ 8.24- 8.13 (m, 3H), 7.86-7.82 (m, 1H), 7.76-7.71 (m, 1H), 7.69- 7.64 (m, 1H), 7.51, 7.49 (each s, combined 1H), 7.31, 7.30 (each s, combined 1H), |
| | | | | | | 7.25 (t, J = 7.4 |
| | | | | | | Hz, 1H), 4.92- |
| | | | | | | 4.78 (m, 1H), |
| | | | | | | 4.09-3.40 (m, |
| | | | | | | 10H), 3.31-3.16 |
| | | | | | | (m, 1H), 2.75- |
| | | | | | | 2.63 (m, 1H), |
| | | | | | | 2.26-2.11 (m, |
| | | | | | | 7H), 2.03-1.69 |
| | | | | | | (m, 3H). MS |
| | | | | | | 553.38 (M + H). |
| 180 | (Z)-1-acetyl-2-((4- (1-methyl-1H- imidazol-2-yl)-6- (morpholine-4- carbonyl)quinolin- 2-yl)methylene)- indolin-3-one | 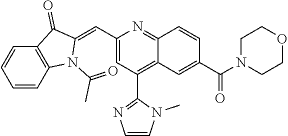 [SMILES: CC(=O)N1/C(=C\c2cc(-c3nccn3C)c3cc(C(=O)N4CCOCC4)ccc3n2)C(=O)c2ccccc21] | Z only | Ex. 2 | Production Ex. 310 CAS No. 16800-68-3 (Reagent Supplier 3) | 1H NMR (500 MHz) δ 8.21- 8.13 (m, 3H), 7.87-7.82 (m, 1H), 7.78 (dd, J = 8.6, 1.9 Hz, 1H), 7.70-7.64 (m, 1H), 7.63 (s, 1H), 7.32 (s, |
| | | | | | | 1H), 7.31 (d, J = |
| | | | | | | 1.3 Hz, 1H), |
| | | | | | | 7.29-7.23 (m, |
| | | | | | | 1H), 7.17 (d, J = |
| | | | | | | 1.3 Hz, 1H), |
| | | | | | | 3.92-3.43 (m, |
| | | | | | | 11H), 2.18 (s, |
| | | | | | | 3H). MS 508.38 |
| | | | | | | (M + H). |
| 181 | (Z)-1-acetyl-2-((4- (morpholinometh- yl)quinolin-2-yl)- methylene)indolin- 3-one | 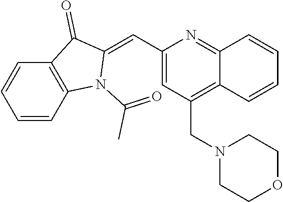 [SMILES: CC(=O)N1/C(=C\c2cc(CN3CCOCC3)c3ccccc3n2)C(=O)c2ccccc21] | Z only | Ex. 2 | Production Ex. 327 CAS No. 16800-68-3 (Reagent Supplier 3) | 1H NMR (500 MHz) δ 8.21 (d, J = 8.5 Hz, 1H), 8.18 (d, J = 8.5 Hz, 1H), 8.12 (d, J = 8.5 Hz, 1H), 7.84 (d, J = 7.6 Hz, 1H), 7.74 (t, J = 7.6 Hz, 1H), 7.68-7.63 (m, 2H), 7.61-7.57 (m, 1H), 7.35 (s, 1H), 7.24 (t, J = |
| | | | | | | 7.6 Hz, 1H), 3.95 |
| | | | | | | (s, 2H), 3.78- |
| | | | | | | 3.74 (m, 4H), |
| | | | | | | 2.60-2.55 (m, |
| | | | | | | 4H), 2.13 (s, |
| | | | | | | 3H). MS 414.3 |
| | | | | | | (M + H). |
| 182 | (Z)-1-acetyl-2-((4- (2-acetyl-1,2,3,4- tetrahydroisoquin- olin-7-yl)-6-(mor- pholine-4-carbon- yl)quinolin-2-yl)- methylene)indolin- 3-one | 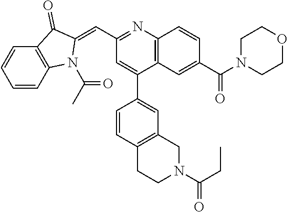 [SMILES: CCC(=O)N1CCc2ccc(-c3cc(/C=C4/C(=O)c5ccccc5N4C(C)=O)nc4ccc(C(=O)N5CCOCC5)cc34)cc2C1] | Z only | Ex. 2 | Production Ex. 328 CAS No. 16800-68-3 (Reagent Supplier 3) | 1H NMR (500 MHz) δ 8.23- 8.16 (m, 2H), 7.96 (dd, J = 14.6, 1.8 Hz, 1H), 7.84 (d, J = 7.5 Hz, 1H), 7.74 (dd, J = 8.6, 1.8 Hz, 1H), 7.70-7.64 (m, 1H), 7.54 (d, J = 6.6 Hz, 1H), 7.39-7.22 (m, |
| | | | | | | 5H), 4.83, 4.73 |
| | | | | | | (each s, com- |
| | | | | | | bined 2H), 3.95- |
| | | | | | | 3.36 (m, 10H), |
| | | | | | | 3.04, 2.98 (each |
| | | | | | | t, each J = 5.9 |
| | | | | | | Hz, combined |
| | | | | | | 2H), 2.23, 2.22 |
| | | | | | | (each s, com- |
| | | | | | | bined 3H), 2.20 |
| | | | | | | (s, 3H). MS |
| | | | | | | 601.4 (M + H). |
| 183 | 4-((2-((1-acetyl-3- oxoindolin-2-yl- idene)methyl)quin- olin-4-yl)methyl)- morpholin-3-one | 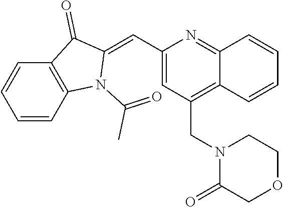 [SMILES: CC(=O)N1/C(=C\c2cc(CN3CCOCC3=O)c3ccccc3n2)C(=O)c2ccccc21] | Z/E Mix. (ca. 20:1) | Ex. 2 | Production Ex. 331 CAS No. 16800-68-3 (Reagent Supplier 3) | 1H NMR (500 MHz) δ 8.19 (d, J = 8.5 Hz, 1H), 8.15 (d, J = 8.5 Hz, 1H), 8.04 (d, J = 8.5 Hz, 1H), 7.84 (d, J = 7.6 Hz, 1H), 7.80- 7.75 (m, 1H), 7.68-7.61 (m, 2H), 7.41 (s, 1H), 7.33 (s, 1H), 7.28-7.23 |
| | | | | | | (m, 1H), 5.18, |
| | | | | | | 5.13 (each s, |
| | | | | | | combined 2H), |
| | | | | | | 4.35, 4.33 (each |
| | | | | | | s, combined 2H), |
| | | | | | | 3.96-3.91 (m, |
| | | | | | | 2H), 3.37-3.33 |
| | | | | | | (m, 2H), 2.73, |
| | | | | | | 2.15 (each s, |
| | | | | | | combined 3H). |
| | | | | | | MS 428.3 |
| | | | | | | (M + H). |
| 184 | 1-acetyl-2-((6- (morpholine-4- carbonyl)-4-(pyr- idin-4-yl)quinolin- 2-yl)methylene)- indolin-3-one | 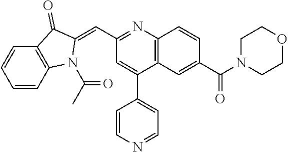 [SMILES: CC(=O)N1/C(=C\c2cc(-c3ccncc3)c3cc(C(=O)N4CCOCC4)ccc3n2)C(=O)c2ccccc21] | Z/E Mix. (ca 9:1) | Ex. 60 | Production Ex. 301 CAS No. 16800-68-3 (Reagent Supplier 3) | 1H NMR (500 MHz) δ 8.85- 8.82 (m, 2H), 8.23 (d, J = 8.5 Hz, 1H), 8.17 (d, J = 8.3 Hz, 1H), 7.88-7.83 (m, 2H), 7.77 (dd, J = 8.5, 1.8 Hz, 1H), 7.70-7.65 |
| | | | | | | (m, 1H), 7.56 |
| | | | | | | (s, 1H), 7.47- |
| | | | | | | 7.44 (m, 2H), |
| | | | | | | 7.34 (s, 1H), |
| | | | | | | 7.29-7.25 (m, |
| | | | | | | 1H), 3.91-3.32 |
| | | | | | | (m, 8H), 2.74, |
| | | | | | | 2.22 (each s, |
| | | | | | | combined 3H). |
| | | | | | | MS 505.32 |
| | | | | | | (M + H) |
| 185 | 1-acetyl-2-((6- (morpholine-4- carbonyl)-4- (pyrimidin-5-yl)- quinolin-2-yl)- methylene) [symbol] -3- [symbol] | 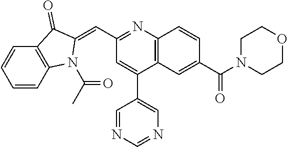 [SMILES: CC(=O)N1/C(=C\c2cc(-c3cncnc3)c3cc(C(=O)N4CCOCC4)ccc3n2)C(=O)c2ccccc21] | Z/E Mix (ca. 14:1) | Ex. 60 | Production Ex. 303 CAS No. 16800-68-3 (Reagent Supplier 3) | 1H NMR (500 MHz) δ 9.42, 9.40 (each s, combined 1H), 9.07, 8.95 (each s, combined 2H), 8.25 (dd, J = 8.6, 0.7 Hz, 1H), 8.17-8.14 (m, |
| | | | | | | 1H), 7.87-7.84 |
| | | | | | | (m, 1H), 7.83- |
| | | | | | | 7.82 (m, 1H), |
| | | | | | | 7.80 (dd, J = |
| | | | | | | 8.6, 1.8 Hz, 1H), |
| | | | | | | 7.71-7.66 (m, |
| | | | | | | 1H), 7.58 (s, |
| | | | | | | 1H), 7.34 (s, |
| | | | | | | 1H), 7.30-7.25 |
| | | | | | | (m, 1H), 3.93- |
| | | | | | | 3.36 (m, 8H), |
| | | | | | | 2.75, 2.23 (each |
| | | | | | | s, combined 3H). |
| | | | | | | MS 506.32 |
| | | | | | | (M + H). |
| 186 | 1-acetyl-2-((4- (morpholine-4- carbonyl)quinolin- 2-yl)methylene)- indolin-3-one | 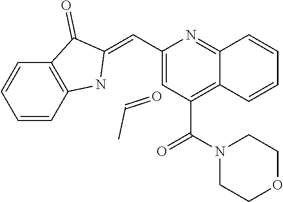 | Z/E Mix (ca. 8:1) | Ex. 60 | Production Ex. 307 CAS No. 16800-68-3 (Reagent Supplier 3) | 1H NMR (500 MHz) δ 8.19- 8.13 (m, 2H), 7.85-7.77 (m, 3H), 7.68-7.61 (m, 2H), 7.50 (s, 1H), 7.30 (s, 1H), 7.27-7.23 (m, 1H), 4.06- 3.98 (m, 1H), 3.94-3.82 (m, 3H), 3.64-3.50 (m, 2H), 3.30- |
| | | | | | | 3.13 (m, 2H), |
| | | | | | | 2.72, 2.17 (each |
| | | | | | | MS 428.22 |
| | | | | | | s, combined 3H). |
| | | | | | | (M + H). |
| 187 | (Z)-1-acetyl-2- ((4-(2-acetyl- 1,2,3,4-tetra- hydroisoquinolin- 6-yl)-quinolin-2- yl)methylene)- indolin-3-one | 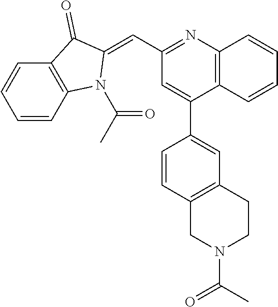 [SMILES: CC(=O)N1CCc2cc(-c3cc(/C=C4/C(=O)c5ccccc5N4C(C)=O)nc4ccccc34)ccc2C1] | Z only | Ex. 60 | Production Ex. 308 CAS No. 16800-68-3 (Reagent Supplier 3) | 1H NMR (500 MHz, dimethyl- sulfoxide-d6) δ 8.08 (d, J = 8.2 Hz, 1H), 8.00 (d, J = 8.4 Hz, 1H), 7.93-7.81 (m, 4H), 7.78 (t, J = 7.4 Hz, 1H), 7.64 (t, J = 7.4 Hz, 1H), 7.46-7.40 (m, 4H), 7.33 (t, J = 7.4 Hz, 1H), 4.78, 4.72 (each s, combined 2H), 3.77-3.71 (m, 2H), 3.01-2.84 (m, 2H), 2.16- |
| | | | | | | 2.10 (m, 6H). |
| | | | | | | MS 488.32 |
| | | | | | | (M + H). |
| 188 | (Z)-1-acetyl-2- ((4-(1-methyl-1H- 1,2,4-triazol-3-yl)- 6-(morpholine-4- carbonyl)quinolin- 2-yl)methylene)- indolin-3-one | 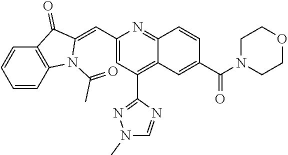 [SMILES: CC(=O)N1/C(=C\c2cc(-c3ncn(C)n3)c3cc(C(=O)N4CCOCC4)ccc3n2)C(=O)c2ccccc21] | Z only | Ex. 60 | Production Ex. 309 CAS No. 16800-68-3 (Reagent Supplier 3) | 1H NMR (500 MHz) δ 9.33 (dd, J =1.8, 0.6 Hz, 1H), 8.37 (s, 1H), 8.24 (d, J = 0.6 Hz, 1H), 8.22-8.17 (m, 2H), 7.86- 7.83 (m, 1H), 7.81 (dd, J = |
| | | | | | | 8.7, 1.9 Hz, 1H), |
| | | | | | | 7.68-7.64 (m, |
| | | | | | | 1H), 7.40 (s, |
| | | | | | | 1H), 7.27-7.23 |
| | | | | | | (m, 1H), 4.11 (s, |
| | | | | | | 3H), 3.96-3.48 |
| | | | | | | (m, 8H), 2.15 (s, |
| | | | | | | 3H). MS 509.33 |
| | | | | | | (M + H). |
| 189 | (Z)-1-acetyl-2-((6- (morpholine-4- carbonyl)-4-(pyr- imidin-2-yl)quin- olin-2-yl)meth- ylene)indolin-3- one | 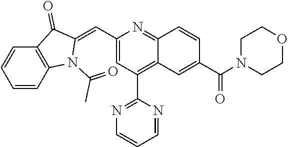 [SMILES: CC(=O)N1/C(=C\c2cc(-c3ncccn3)c3cc(C(=O)N4CCOCC4)ccc3n2)C(=O)c2ccccc21] | Z only | Ex. 60 | Production Ex. 311 CAS No. 16800-68-3 (Reagent Supplier 3) | 1H NMR (500 MHz) δ 9.04- 8.95 (m, 3H), 8.33-8.28 (m, 1H), 8.26-8.16 (m, 2H), 7.88- 7.76 (m, 2H), 7.70-7.63 (m, 1H), 7.50-7.39 |
| | | | | | | (m, 2H), 7.30- |
| | | | | | | 7.22 (m, 1H), |
| | | | | | | 3.96-3.39 (m, |
| | | | | | | 8H), 2.20-2.14 |
| | | | | | | (m, 3H). MS |
| | | | | | | 506.27 (M + H). |
| 190 | (Z)-6-(2-((1-acet- yl-3-oxoindolin- 2-ylidene)meth- yl)quinolin-4-yl)- 3,4-dihydroiso- quinolin-1(2H)-one | 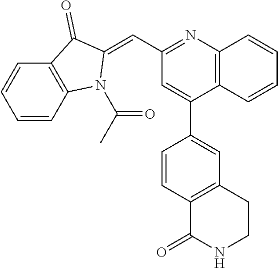 [SMILES: CC(=O)N1/C(=C\c2cc(-c3ccc4c(c3)CCNC4=O)c3ccccc3n2)C(=O)c2ccccc21] | Z only | Ex. 60 | Production Ex. 361 CAS No. 16800-68-3 (Reagent Supplier 3) | 1H NMR (500 MHz) δ 8.26 (d, J = 8.3 Hz, 1H), 8.21 (d, J = 8.3 Hz, 1H), 8.19 (t, J = 8.3 Hz, 1H), 7.88-7.83 (m, 2H), 7.79-7.75 (m, 1H), 7.68- 7.64 (m, 1H), 7.58-7.51 (m, 3H), 7.40 (s, 1H), 7.36 (s, 1H), 7.27-7.23 (m, 1H), 6.51 (s, 1H), 3.71-3.67 (m, 2H), 3.13 (t, |
| | | | | | | J = 6.6 Hz, 2H), |
| | | | | | | 2.21 (s, 3H). MS |
| | | | | | | 460.19 (M + H). |
| 191 | (Z)-1-acetyl-2- ((6-(morpholine- 4-carbonyl)-4- (pyridazin-3-yl)- quinolin-2-yl)- methylene)indolin- 3-one | 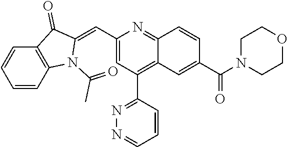 [SMILES: CC(=O)N1/C(=C\c2cc(-c3cccnn3)c3cc(C(=O)N4CCOCC4)ccc3n2)C(=O)c2ccccc21] | Z only | Ex. 60 | Production Ex. 315 CAS No. 16800-68-3 (Reagent Supplier 3) | 1H NMR (500 MHz) δ 9.39 (dd, J = 5.1, 1.7 Hz, 1H), 8.24 (d, J = 8.7 Hz, 1H), 8.18 (d, J = 8.4 Hz, 1H), 8.16 (d, J = 1.9 Hz, 1H), 7.88-7.74 (m, |
| | | | | | | 5H), 7.70-7.65 |
| | | | | | | (m, 1H), 7.36 |
| | | | | | | (s, 1H), 7.28- |
| | | | | | | 7.23 (m, 1H), |
| | | | | | | 3.92-3.41 (m, |
| | | | | | | 8H), 2.20 (s, |
| | | | | | | 3H). MS 506.17 |
| | | | | | | (M + H). |
| 192 | (Z)-1-acetyl-2- ((4-(1-methyl- 1H-imidazol-4- yl)-6-(morpholine- 4-carbonyl)quin- olin-2-yl)meth- ylene)indolin-3- one | 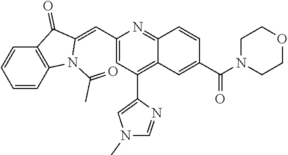 [SMILES: CC(=O)N1/C(=C\c2cc(-c3cn(C)cn3)c3cc(C(=O)N4CCOCC4)ccc3n2)C(=O)c2ccccc21] | Z only | Ex. 60 | Production Ex. 316 CAS No. 16800-68-3 (Reagent Supplier 3) | 1H NMR (500 MHz) δ 8.87 (d, J = 1.8 Hz, 1H), 8.20 (d, J = 8.3 Hz, 1H), 8.13 (d, J = 8.6 Hz, 1H), 7.85 (s, 1H), 7.84 (dd, J = 7.5, 1.3 Hz, 1H), 7.75 (dd, |
| | | | | | | J = 8.6, 1.9 Hz, |
| | | | | | | 1H), 7.68-7.63 |
| | | | | | | (m, 2H), 7.43 |
| | | | | | | (d, J = 1.3 Hz, |
| | | | | | | 1H), 7.35 (s, |
| | | | | | | 1H), 7.26-7.22 |
| | | | | | | (m, 1H), 3.96- |
| | | | | | | 3.46 (m, 11H), |
| | | | | | | 2.13 (s, 3H). MS |
| | | | | | | 508.20 (M + H). |
| 193 | (Z)-1-acetyl-2- ((4-(2-acetyl- 1,2,3,4-tetra- hydroisoquinolin- 5-yl)quinolin-2- yl)methylene)- indolin-3-one | 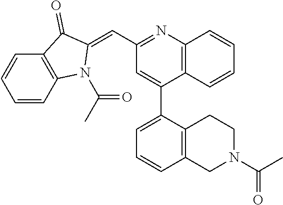 [SMILES: CC(=O)N1CCc2c(cccc2-c2cc(/C=C3/C(=O)c4ccccc4N3C(C)=O)nc3ccccc23)C1] | Z only | Ex. 60 | Production Ex. 317 CAS No. 16800-68-3 (Reagent Supplier 3) | 1H NMR (500 MHz) δ 8.23- 8.15 (m, 2H), 7.86-7.83 (m, 1H), 7.78-7.73 (m, 1H), 7.68- 7.64 (m, 1H), 7.53-7.16 (m, 8H), 4.97-4.70 (m, 2H), 3.76- 3.48 (m, 2H), 2.57-2.34 (m, 2H), 2.24-2.11 |
| | | | | | | (m, 6H). MS |
| | | | | | | 488.45 (M + H). |
| 194 | (Z)-1-acetyl-2- ((6-(morpholine- 4-carbonyl)-4- (pyrazin-2-yl)- quinolin-2-yl)- methylene)indolin- 3-one | 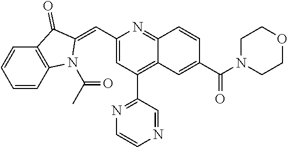 [SMILES: CC(=O)N1/C(=C\c2cc(-c3cnccn3)c3cc(C(=O)N4CCOCC4)ccc3n2)C(=O)c2ccccc21] | Z only | Ex. 60 | Production Ex. 319 CAS No. 16800-68-3 (Reagent Supplier 3) | 1H NMR (500 MHz) δ 8.99 (d, J = 1.5 Hz, 1H), 8.83 (dd, J = 2.5, 1.5 Hz, 1H), 8.78 (d, J = 2.5 Hz, 1H), 8.25 (d, J = 1.8 Hz, 1H), 8.23 (d, J = 8.7 Hz, |
| | | | | | | 1H), 8.18 (d, J = |
| | | | | | | 8.3 Hz, 1H), 7.85 |
| | | | | | | (dd, J = 7.6, 1.4 |
| | | | | | | Hz, 1H), 7.80 |
| | | | | | | (dd, J = 8.6, 1.8 |
| | | | | | | Hz, 1H), 7.78 (s, |
| | | | | | | 1H), 7.70-7.65 |
| | | | | | | (m, 1H), 7.37 (s, |
| | | | | | | 1H), 7.29-7.24 |
| | | | | | | (m, 1H), 3.94- |
| | | | | | | 3.41 (m, 8H), |
| | | | | | | 2.20 (s, 3H). MS |
| | | | | | | 506.2 (M + H). |
| 195 | (Z)-1-acetyl-2- ((6-(morpholine- 4-carbonyl)-5- (naphthalen-1-yl)- quinolin-2-yl)- methylene)indolin- 3-one | 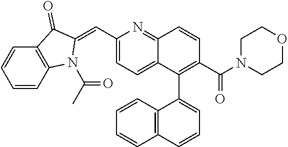 [SMILES: CC(=O)N1/C(=C\c2ccc3c(-c4cccc5ccccc45)c(C(=O)N4CCOCC4)ccc3n2)C(=O)c2ccccc21] | Z only | Ex. 60 | Production Ex. 320 CAS No. 16800-68-3 (Reagent Supplier 3) | 1H NMR (500 MHz) δ 8.26 (d, J = 8.7 Hz, 1H), 8.20 (d, J = 8.3 Hz, 1H), 8.00 (t, J = 8.1 Hz, 2H), 7.84 (dd, J = 7.6, 1.2 Hz, 1H), 7.77 (dd, |
| | | | | | | J = 12.1, 8.6 Hz, |
| | | | | | | 1H), 7.72-7.60 |
| | | | | | | (m, 4H), 7.52- |
| | | | | | | 7.46 (m, 1H), |
| | | | | | | 7.42 (d, J = 8.7 |
| | | | | | | Hz, 1H), 7.26 (h, |
| | | | | | | J = 8.1 Hz, 3H), |
| | | | | | | 7.11 (d, J = 8.6 |
| | | | | | | Hz, 1H), 3.64- |
| | | | | | | 2.67 (m, 8H), |
| | | | | | | 2.21 (s, 3H). MS |
| | | | | | | 554.3 (M + H). |
| 196 | (Z)-1-acetyl-2-((4- (2-(1-methyl-1H- pyrazol-4-yl)phen- yl)-6-(morpholine- 4-carbonyl)quin- olin-2-yl)meth- ylene)-indolin-3- one | 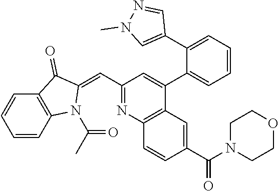 [SMILES: CC(=O)N1/C(=C\c2cc(-c3ccccc3-c3cnn(C)c3)c3cc(C(=O)N4CCOCC4)ccc3n2)C(=O)c2ccccc21] | Z only | Ex. 60 | Production Ex. 321 CAS No. 16800-68-3 (Reagent Supplier 3) | 1H NMR (500 MHz) δ 8.20- 8.14 (m, 2H), 7.84 (dd, J = 7.5, 1.3 Hz, 1H), 7.75-7.63 (m, 2H), 7.60 (dd, J = 7.6, 1.3 Hz, 1H), 7.57-7.50 (m, 3H), 7.45- 7.39 (m, 1H), 7.32-7.20 (m, |
| | | | | | | 3H), 7.04 (s, |
| | | | | | | 1H), 6.82 (s, |
| | | | | | | 1H), 3.93-3.09 |
| | | | | | | (m, 11H), 2.18 |
| | | | | | | (s, 3H). MS |
| | | | | | | 584.3 (M + H). |
| 197 | 1-acetyl-2-((6- (morpholine-4- carbonyl)-8- (naphthalen-1-yl)- quinolin-2-yl)- methylene)indolin- 3-one | 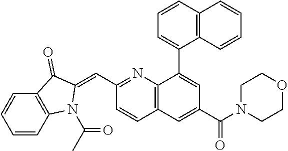 [SMILES: CC(=O)N1/C(=C\c2ccc3cc(C(=O)N4CCOCC4)cc(-c4cccc5ccccc45)c3n2)C(=O)c2ccccc21] | Z/E Mix. (ca. 4:3) | Ex. 60 | Production Ex. 322 CAS No. 16800-68-3 (Reagent Supplier 3) | 1H NMR (500 MHz) δ 8.35- 7.14 (m, 16H), 4.02-3.45 (m, 8H), 2.50, 1.61 (each s, com- bined 3H). MS 554.3 (M + H). |
| 198 | (Z)-1-acetyl-2- ((3-(1-methyl-1H- pyrazol-4-yl)-6- (morpholine-4- carbonyl)quinolin- 2-yl)methylene)- indolin-3-one | 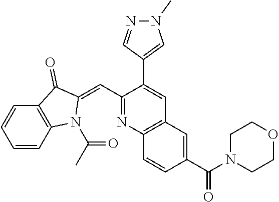 [SMILES: CC(=O)N1/C(=C\c2nc3ccc(C(=O)N4CCOCC4)cc3cc2-c2cnn(C)c2)C(=O)c2ccccc21] | Z only | Ex. 60 | Production Ex. 323 CAS No. 16800-68-3 (Reagent Supplier 3) | 1H NMR (500 MHz) δ 8.18 (s, 1H), 8.13 (d, J = 8.3 Hz, 1H), 8.09 (d, J = 8.6 Hz, 1H), 7.88 (d, J = 1.8 Hz, 1H), 7.82 (dd, J = 7.6, 1.5 Hz, 1H), 7.77 (s, 1H), 7.73-7.60 (m, 3H), 7.51 (s, 1H), 7.24 (t, J = 7.3 Hz, 1H), 4.04 |
| | | | | | | (s, 3H), 3.93-3.40 |
| | | | | | | (m, 8H), 2.10 (s, |
| | | | | | | 3H). MS 508.3 |
| | | | | | | (M + H). |
| 199 | (Z)-1-acetyl-2-((5- (1-methyl-1H-pyr- azol-4-yl)-6- (morpholine-4- carbonyl)quinolin- 2-yl)methylene)- indolin-3-one | 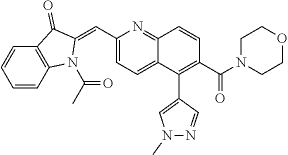 [SMILES: CC(=O)N1/C(=C\c2ccc3c(-c4cnn(C)c4)c(C(=O)N4CCOCC4)ccc3n2)C(=O)c2ccccc21] | Z only | Ex. 60 | Production Ex. 324 CAS No. 16800-68-3 (Reagent Supplier 3) | 1H NMR (500 MHz) δ 8.41 (d, J = 8.6 Hz, 1H), 8.19 (d, J = 8.2 Hz, 1H), 8.11 (d, J = 8.6 Hz, 1H), 7.86-7.82 (m, 1H), 7.73-7.55 (m, 5H), 7.32 (s, 1H), 7.27-7.23 |
| | | | | | | (m, 1H), 4.04 (s, |
| | | | | | | 3H), 3.77-3.59 |
| | | | | | | (m, 3H), 3.49- |
| | | | | | | 3.38 (m, 2H), |
| | | | | | | 3.15-3.06 (m, |
| | | | | | | 1H), 3.02-2.88 |
| | | | | | | (m, 2H), 2.15 |
| | | | | | | (s, 3H). MS |
| | | | | | | 508.3 (M + H). |
| 200 | (Z)-1-acetyl-2- ((5-(2-acetyl- 1,2,3,4-tetra- hydroisoquinolin- 6-yl)-6-(morph- oline-4-carbonyl)- quinolin-2-yl)- methylene)indolin- 3-one | 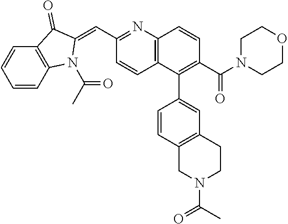 [SMILES: CC(=O)N1CCc2cc(-c3c(C(=O)N4CCOCC4)ccc4nc(/C=C5/C(=O)c6ccccc6N5C(C)=O)ccc34)ccc2C1] | Z only | Ex. 60 | Production Ex. 329 CAS No. 16800-68-3 (Reagent Supplier 3) | 1H NMR (500 MHz) δ 8.21- 8.06 (m, 3H), 7.84 (dd, J = 7.6, 1.3 Hz, 1H), 7.72-7.63 (m, 2H), 7.56 (dd, J = 8.9, 4.7 Hz, 1H), 7.44-7.22 (m, 4H), 7.17- 7.06 (m, 1H), 4.91-4.68 (m, 2H), 3.95-3.85 (m, 1H), 3.81- |
| | | | | | | 3.71 (m, 1H), |
| | | | | | | 3.70-3.40 (m, |
| | | | | | | 4H), 3.37-3.28 |
| | | | | | | (m, 1H), 3.24- |
| | | | | | | 3.12 (m, 1H), |
| | | | | | | 3.08-2.83 (m, |
| | | | | | | 4H), 2.27-2.20 |
| | | | | | | (m, 3H), 2.16 |
| | | | | | | (s, 3H). MS |
| | | | | | | 601.3 (M + H). |
| 201 | (Z)-1-acetyl-2- ((5-(2-acetyl- 1,2,3,4-tetra- hydroisoquinolin- 7-yl)-6-(morph- oline-4-carbonyl)- quinolin-2-yl)- methylene)indolin- 3-one | 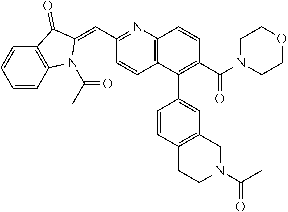 [SMILES: CC(=O)N1CCc2ccc(-c3c(C(=O)N4CCOCC4)ccc4nc(/C=C5/C(=O)c6ccccc6N5C(C)=O)ccc34)cc2C1] | Z only | Ex. 60 | Production Ex. 330 CAS No. 16800-68-3 (Reagent Supplier 3) | 1H NMR (500 MHz) δ 8.21- 8.07 (m, 3H), 7.84 (d, J = 7.5 Hz, 1H), 7.72- 7.63 (m, 2H), 7.59-7.51 (m, 1H), 7.44-7.21 (m, 4H), 7.17- 7.04 (m, 1H), 4.99-4.59 (m, 2H), 3.99-3.71 (m, 2H), 3.70- |
| | | | | | | 3.39 (m, 4H), |
| | | | | | | 3.37-3.27 (m, |
| | | | | | | 1H), 3.21-3.13 |
| | | | | | | (m, 1H), 3.06- |
| | | | | | | 2.90 (m, 4H), |
| | | | | | | 2.25-2.18 (m, |
| | | | | | | 3H), 2.17 (s, |
| | | | | | | 3H). MS 601.3 |
| | | | | | | (M + H). |
| 202 | (Z)-2-((1-acetyl- 3-oxoindolin-2- ylidene)methyl)-3- (naphthalen-1-yl)- quinoline-6- carboxylic acid | 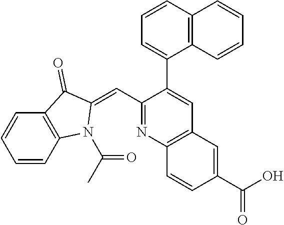 [SMILES: CC(=O)N1/C(=C\c2nc3ccc(C(=O)O)cc3cc2-c2cccc3ccccc23)C(=O)c2ccccc21] | Z only | Ex. 53 | Production Ex. 293 CAS No. 16800-68-3 (Reagent Supplier 3) | 1H NMR (500 MHz) δ 8.66 (d, J = 1.9 Hz, 1H), 8.40 (dd, J = 8.9, 1.9 Hz, 1H), 8.30 (d, J = 0.8 Hz, 1H), 8.28-8.24 (m, 1H), 8.13- 8.09 (m, 1H), 8.06-7.98 (m, 2H), 7.73-7.69 (m, 1H), 7.66- 7.60 (m, 2H), 7.57-7.53 (m, |
| | | | | | | 1H), 7.49 (dd, |
| | | | | | | J = 7.0, 1.2 Hz, |
| | | | | | | 1H), 7.45-7.37 |
| | | | | | | (m, 2H), 7.21- |
| | | | | | | 7.16 (m, 1H), |
| | | | | | | 6.92 (s, 1H), |
| | | | | | | 2.26 (s, 3H). |
| | | | | | | MS 485.28 |
| | | | | | | (M + H). |
| 203 | (Z)-1-acetyl-2- ((4-(1-methyl-1H- pyrazol-4-yl)quin- azolin-2-yl)meth- ylene)indolin-3- one | 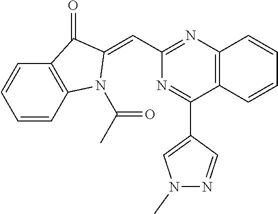 [SMILES: CC(=O)N1/C(=C\c2nc(-c3cnn(C)c3)c3ccccc3n2)C(=O)c2ccccc21] | Z only | Ex. 53 | Production Ex. 318 CAS No. 16800-68-3 (Reagent Supplier 3) | 1H NMR (500 MHz) δ 8.39 (d, J = 8.4 Hz, 1H), 8.26 (d, J = 8.3 Hz, 1H), 8.25 (s, 1H), 8.18 (s, 1H), 8.07 (d, J = 8.4 Hz, 1H), 7.95- 7.91 (m, 1H), 7.85 (d, J = 7.5 Hz, 1H), 7.72- 7.64 (m, 2H), 7.47 (s, 1H), |
| | | | | | | 7.29-7.24 (m, |
| | | | | | | 1H), 4.09 (s, |
| | | | | | | 3H), 2.11 (s, |
| | | | | | | 3H). MS 396.1 |
| | | | | | | (M + H). |
| 204 | 1-acetyl-2-((4-(4- (oxetan-3-yl)piper- azine-1-carbonyl)- quinolin-2-yl)- methylene)indolin- 3-one | 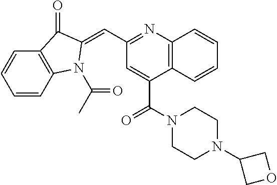 [SMILES: CC(=O)N1/C(=C\c2cc(C(=O)N3CCN(C4COC4)CC3)c3ccccc3n2)C(=O)c2ccccc21] | Z/E Mix. (ca. 13:1) | Ex. 115 | Production Ex. 312 CAS No. 16800-68-3 (Reagent Supplier 3) | 1H NMR (500 MHz, dimethyl- sulfoxide-d6) δ 8.17-8.13 (m, 2H), 7.96, 7.50 (each s, com- bined 1H), 7.84- 7.76 (m, 3H), 7.67-7.60 (m, 2H), 7.30, 7.29 (each s, com- bined 1H), 7.24 |
| | | | | | | (t, J = 7.4 Hz, |
| | | | | | | 1H), 4.69-4.56 |
| | | | | | | (m, 4H), 4.06- |
| | | | | | | 3.93 (m, 2H), |
| | | | | | | 3.59-3.49 (m, |
| | | | | | | 1H), 3.32-3.20 |
| | | | | | | (m, 2H), 2.71 (s, |
| | | | | | | 0.2H), 2.55-2.46 |
| | | | | | | (m, 2H), 2.28- |
| | | | | | | 2.13 (m, 4.8H). |
| | | | | | | MS 483.29 |
| | | | | | | (M + H). |
| 205 | (Z)-1-acetyl-2- ((4-(1-methyl-1H- pyrazol-4-yl)-6- (((tetrahydro-2H- pyran-4-yl)amino)- methyl)quinolin-2- yl)methylene)- indolin-3-one | 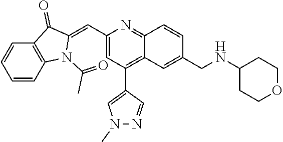 [SMILES: CC(=O)N1/C(=C\c2cc(-c3cnn(C)c3)c3cc(CNC4CCOCC4)ccc3n2)C(=O)c2ccccc21] | Z only | Ex. 130 | Production Ex. 392 | 1H NMR (500 MHz) δ 8.23- 8.18 (m, 1H), 8.12-8.05 (m, 2H), 7.86-7.80 (m, 2H), 7.79- 7.72 (m, 2H), 7.68-7.62 (m, 1H), 7.53-7.49 |
| | | | | | | (m, 1H), 7.34- |
| | | | | | | 7.29 (m, 1H), |
| | | | | | | 7.26-7.21 (m, |
| | | | | | | 1H), 4.07 (s, |
| | | | | | | 3H), 4.02-3.95 |
| | | | | | | (m, 4H), 3.44- |
| | | | | | | 3.35 (m, 2H), |
| | | | | | | 2.82-2.73 (m, |
| | | | | | | 1H), 2.14 (s, |
| | | | | | | 3H), 1.93-1.85 |
| | | | | | | (m, 2H), 1.53- |
| | | | | | | 1.41 (m, 2H). |
| | | | | | | MS 508.37 |
| | | | | | | (M + H). |
| 206 | 1-acetyl-2-((4-(2- (1-methyl-1H- pyrazol-4-yl)phen- yl)-6-(((tetrahydro- 2H-pyran-4-yl)- amino)methyl)- quinolin-2-yl)- methylene)indolin- 3-one | 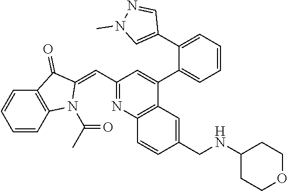 [SMILES: CC(=O)N1/C(=C\c2cc(-c3ccccc3-c3cnn(C)c3)c3cc(CNC4CCOCC4)ccc3n2)C(=O)c2ccccc21] | Z/E Mix. (ca. 8:1) | Ex. 130 | Production Ex. 395 | 1H NMR (500 MHz) δ 8.19 (d, J = 8.3 Hz, 1H), 8.10 (d, J = 8.6 Hz, 1H), 7.84 (dd, J = 7.6, 1.4 Hz, 1H), 7.75- 7.58 (m, 3H), 7.56-7.50 (m, 1H), 7.45-7.38 (m, 3H), 7.33- 7.28 (m, 2H), |
| | | | | | | 7.28-7.21 (m, |
| | | | | | | 1H), 7.12 (s, |
| | | | | | | 1H), 6.71 (s, |
| | | | | | | 1H), 3.98-3.90 |
| | | | | | | (m, 2H), 3.86 |
| | | | | | | (s, 2H), 3.59 (s, |
| | | | | | | 3H), 3.37-3.28 |
| | | | | | | (m, 2H), 2.70- |
| | | | | | | 2.60 (m, 1H), |
| | | | | | | 2.74, 2.16 (each |
| | | | | | | s, combined |
| | | | | | | 3H), 1.81-1.70 |
| | | | | | | (m, 2H), 1.47- |
| | | | | | | 1.34 (m, 2H). |
| | | | | | | MS 584.3 |
| | | | | | | (M + H). |
| 207 | 1-acetyl-2-((4-(2- acetyl-1,2,3,4- tetrahydroisoquin- olin-5-yl)-6- (((tetrahydro-2H- pyran-4-yl)amino)- methyl)quinolin- 2-yl)methylene)- indolin-3-one | 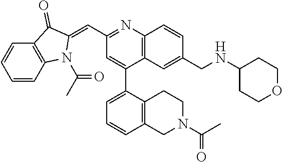 [SMILES: CC(=O)N1CCc2c(cccc2-c2cc(/C=C3/C(=O)c4ccccc4N3C(C)=O)nc3ccc(CNC4CCOCC4)cc23)C1] | Z/E Mix. (ca. 9:2) | Ex. 130 | Production Ex. 396 | 1H NMR (500 MHz) δ 8.23- 7.96 (m, 2H), 7.88-7.62 (m, 3H), 7.45-7.13 (m, 7H), 4.98- 4.69 (m, 2H), 3.99-3.88 (m, 4H), 3.73-3.48 (m, 2H), 3.35 |
| | | | | | | (t, J = 11.5 Hz, |
| | | | | | | 2H), 2.74-2.67 |
| | | | | | | (m, 1H), 2.61- |
| | | | | | | 2.33 (m, 2H), |
| | | | | | | 2.23, 2.22 (each |
| | | | | | | s, combined |
| | | | | | | 3H), 2.73, 2.12 |
| | | | | | | (each s, com- |
| | | | | | | bined 3H), 1.84- |
| | | | | | | 1.77 (m, 2H), |
| | | | | | | 1.47-1.36 (m, |
| | | | | | | 2H). MS 601.4 |
| | | | | | | (M + H). |
| 208 | (Z)-1-acetyl-2- ((4-(((tetrahydro- 2H-pyran-4-yl)- amino)methyl)- quinolin-2-yl)- methylene)indolin- 3-one | 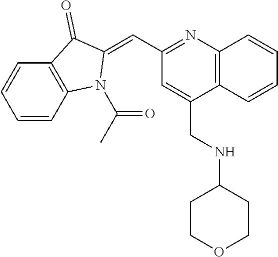 [SMILES: CC(=O)N1/C(=C\c2cc(CNC3CCOCC3)c3ccccc3n2)C(=O)c2ccccc21] | Z only | Ex. 130 | Production Ex. 397 | 1H NMR (500 MHz) δ 8.21 (d, J = 8.5 Hz, 1H), 8.13 (d, J = 8.5 Hz, 1H), 8.05 (d, J = 8.5 Hz, 1H), 7.84 (dd, J = 7.5, 1.3 Hz, 1H), 7.76-7.71 (m, 2H), 7.67-7.63 (m, 1H), 7.62- 7.58 (m, 1H), 7.35 (s, 1H), 7.24 (t, J = 7.5 Hz, 1H), 4.33 (s, 2H), 4.06-4.01 |
| | | | | | | (m, 2H), 3.50- |
| | | | | | | 3.41 (m, 2H), |
| | | | | | | 2.91-2.84 (m, |
| | | | | | | 1H), 2.12 (s, |
| | | | | | | 3H), 2.00-1.94 |
| | | | | | | (m, 2H), 1.59- |
| | | | | | | 1.49 (m, 2H). |
| | | | | | | MS 428.4 |
| | | | | | | (M + H). |
| 209 | (Z)-1-acetyl-2- ((4-(2-acetyl- 1,2,3,4-tetrahydro- isoquinolin-6-yl)- 6-(((tetrahydro- 2H-pyran-4-yl)- amino)methyl)- quinolin-2-yl)- methylene)indolin- 3-one | 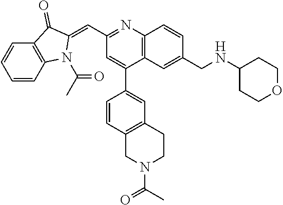 [SMILES: CC(=O)N1CCc2cc(-c3cc(/C=C4/C(=O)c5ccccc5N4C(C)=O)nc4ccc(CNC5CCOCC5)cc34)ccc2C1] | Z only | Ex. 130 | Production Ex. 398 | 1H NMR (500 MHz) δ 8.22 (d, J = 8.3 Hz, 1H), 8.13 (dd, J = 8.6, 1.8 Hz, 1H), 7.84 (dd, J = 7.5, 1.3 Hz, 1H), 7.80- 7.72 (m, 2H), 7.69-7.63 (m, 1H), 7.49 (s, 1H), 7.39-7.28 (m, 4H), 7.27- 7.22 (m, 1H), |
| | | | | | | 4.87, 4.76 (each |
| | | | | | | s, combined |
| | | | | | | 2H), 4.01-3.89 |
| | | | | | | (m, 5H), 3.79 (t, |
| | | | | | | J = 5.9 Hz, 1H), |
| | | | | | | 3.42-3.33 (m, |
| | | | | | | 2H), 3.02, 2.96 |
| | | | | | | (each t, each J = |
| | | | | | | 5.9 Hz, com- |
| | | | | | | bined 2H), 2.78- |
| | | | | | | 2.69 (m, 1H), |
| | | | | | | 2.25, 2.23 (each |
| | | | | | | s, combined |
| | | | | | | 3H), 2.18 (s, |
| | | | | | | 3H), 1.88-1.81 |
| | | | | | | (m, 2H), 1.49- |
| | | | | | | 1.39 (m, 2H). |
| | | | | | | MS 601.4 |
| | | | | | | (M + H). |
| 210 | (Z)-1-acetyl-2-((4- (3-(1-methyl-1H- pyrazol-4-yl)- phenyl)-6-(((tetra- hydro-2H-pyran- 4-yl)amino)meth- yl)quinolin-2-yl)- methylene)indolin- 3-one | 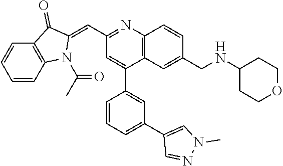 [SMILES: CC(=O)N1/C(=C\c2cc(-c3cccc(-c4cnn(C)c4)c3)c3cc(CNC4CCOCC4)ccc3n2)C(=O)c2ccccc21] | Z only | Ex. 130 | Production Ex. 399 | 1H NMR (500 MHz) δ 8.22 (dd, J = 8.3, 0.9 Hz, 1H), 8.14 (d, J = 8.7 Hz, 1H), 7.86-7.79 (m, 3H), 7.77 (dd, J = 8.7, 1.9 Hz, 1H), 7.70 (s, 1H), 7.69-7.59 |
| | | | | | | (m, 3H), 7.57- |
| | | | | | | 7.51 (m, 2H), |
| | | | | | | 7.39-7.34 (m, |
| | | | | | | 2H), 7.28-7.22 |
| | | | | | | (m, 1H), 3.99- |
| | | | | | | 3.90 (m, 7H), |
| | | | | | | 3.39-3.30 (m, |
| | | | | | | 2H), 2.77-2.67 |
| | | | | | | (m, 1H), 2.19 (s, |
| | | | | | | 3H), 1.86-1.78 |
| | | | | | | (m, 2H), 1.47- |
| | | | | | | 1.36 (m, 2H). |
| | | | | | | MS 584.4 |
| | | | | | | (M + H). |
| 211 | (Z)-1-acetyl-2-((4- (2-acetyl-1,2,3,4- tetrahydroisoquin- olin-7-yl)-6- (((tetrahydro-2H- pyran-4-yl)amino)- methyl)quinolin- 2-yl)methylene)- indolin-3-one | 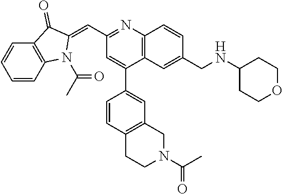 [SMILES: CC(=O)N1CCc2ccc(-c3cc(/C=C4/C(=O)c5ccccc5N4C(C)=O)nc4ccc(CNC5CCOCC5)cc34)cc2C1] | Z only | Ex. 130 | Production Ex. 384 | 1H NMR (500 MHz) δ 8.21 (d, J = 8.3 Hz, 1H), 8.13 (dd, J = 8.6, 3.3 Hz, 1H), 7.84 (d, J = 7.5 Hz, 1H), 7.80-7.74 (m, 2H), 7.69- 7.62 (m, 1H), 7.49 (d, J = 7.9 Hz, 1H), 7.39- 7.28 (m, 4H), |
| | | | | | | 7.28-7.21 (m, |
| | | | | | | 1H), 4.84, 4.73 |
| | | | | | | (each s, com- |
| | | | | | | bined 2H), 4.01- |
| | | | | | | 3.90 (m, 5H), |
| | | | | | | 3.79 (t, J = 5.9 |
| | | | | | | Hz, 1H), 3.42- |
| | | | | | | 3.33 (m, 2H), |
| | | | | | | 3.04, 2.98 (each |
| | | | | | | t, each J = 6.0 |
| | | | | | | Hz, combined |
| | | | | | | 2H), 2.79-2.69 |
| | | | | | | (m, 1H), 2.23, |
| | | | | | | 2.23 (each s, |
| | | | | | | combined 3H), |
| | | | | | | 2.19 (s, 3H), |
| | | | | | | 1.88-1.81 (m, |
| | | | | | | 2H), 1.49-1.39 |
| | | | | | | (m, 2H). MS |
| | | | | | | 601.5 (M + H). |
| 212 | 1-acetyl-2-((6- (morpholine-4- carbonyl)-4-(1H- 1,2,4-triazol-3-yl)- quinolin-2-yl)- methylene)indolin- 3-one | 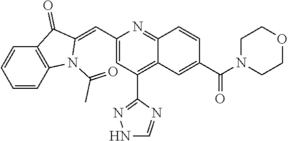 [SMILES: CC(=O)N1/C(=C\c2cc(-c3nc[nH]n3)c3cc(C(=O)N4CCOCC4)ccc3n2)C(=O)c2ccccc21] | Z/E Mix. (ca. 3:1) | Ex. 129 | Production Ex. 393 | 1H NMR (500 MHz) δ 9.48- 9.33 (m, 1H), 8.80 (s, 0.23H), 8.29-8.22 (m, 1.75H), 8.16 (d, J = 8.5 Hz, 1.78H), 8.03 (d, 0.23H), 7.88- |
| | | | | | | 7.58 (m, 3.29H), |
| | | | | | | 7.33 (s, 0.75H), |
| | | | | | | 7.26-7.15 (m, |
| | | | | | | 1H), 4.02-3.48 |
| | | | | | | (m, 8H), 2.69, |
| | | | | | | 2.14 (each s, |
| | | | | | | combined 3H). |
| | | | | | | MS 495.2 |
| | | | | | | (M + H). |
| 213 | (Z)-2-((4-(1H- pyrazol-4-yl)quin- olin-2-yl)meth- ylene)-1-acetyl- indolin-3-one | 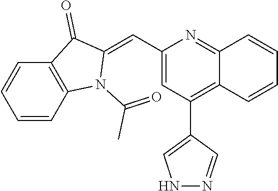 [SMILES: CC(=O)N1/C(=C\c2cc(-c3cn[nH]c3)c3ccccc3n2)C(=O)c2ccccc21] | Z only | Ex. 129 | Production Ex. 394 | 1H NMR (500 MHz) δ 8.22 (dd, J = 8.3, 0.8 Hz, 1H), 8.17-8.13 (m, 2H), 8.06- 7.98 (m, 2H), 7.85 (dd, J = 7.5, 1.3 Hz, 1H), 7.79-7.73 (m, 1H), 7.69-7.63 (m, 1H), 7.60- 7.53 (m, 2H), |
| | | | | | | 7.37-7.33 (m, |
| | | | | | | 1H), 7.28-7.22 |
| | | | | | | (m, 1H), 2.17 |
| | | | | | | (s, 3H). MS |
| | | | | | | 381.17 (M + H). |
| 214 | (Z)-1-acetyl-2- ((3-(naphthalen-1- yl)-6-(1H-tetrazol- 5-yl)quinolin-2- yl)methylene)- indolin-3-one | 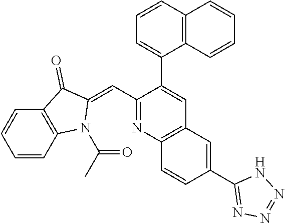 [SMILES: CC(=O)N1/C(=C\c2nc3ccc(-c4nnn[nH]4)cc3cc2-c2cccc3ccccc23)C(=O)c2ccccc21] | Z only | Ex. 129 | Production Ex. 383 | 1H NMR (500 MHz, dimethyl- sulfoxide-d6) δ 8.74 (d, J = 1.9 Hz, 1H), 8.61 (s, 1H), 8.52 (dd, J = 8.8, 1.9 Hz, 1H), 8.32 (s, 1H), 8.19-8.11 (m, 3H), 8.04 (d, J = 8.3 Hz, 1H), 7.77-7.72 (m, 2H), 7.69- 7.59 (m, 3H), |
| | | | | | | 7.51-7.44 (m, |
| | | | | | | 2H), 7.26 (t, J = |
| | | | | | | 7.4 Hz, 1H), |
| | | | | | | 6.62 (s, 1H), |
| | | | | | | 2.20 (s, 3H). |
| | | | | | | MS 509.3 |
| | | | | | | (M + H). |

[symbol]

indicates data missing or illegible when filed

When the following synthetic literatures are mentioned in the texts and tables, the concerned compounds were synthesized according to the descriptions of the synthetic literatures.

| | |
|---|---|
| Synthetic Literature 1 | Wozniak et al., Organic Letters, 22(13), 4970-4973; 2020 |
| Synthetic Literature 2 | Holton et al., Tetrahedron Letters, 18(6), 533-534, 1977 |
| Synthetic Literature 3 | Brodney et al., WO2011125006 |
| Synthetic Literature 4 | Li et al., Organic Letters, 14(21), 5420-5423; 2012 |
| Synthetic Literature 5 | Kim et al., Bioorganic & Medicinal Chemistry Letters, 20(1), 413-417; 2010 |
| Synthetic Literature 6 | Boyd et al., Tetrahedron Letters, 55(30), 4117-4119; 2014 |
| Synthetic Literature 7 | Guo et al., WO2019210828 |
| Synthetic Literature 8 | Maccari et al., European Journal of Medicinal Chemistry, 81, 1-14; 2014 |
| Synthetic Literature 9 | Kobayashi et al., Heterocycles, 92(6), 1063-1074, 2016 |
| Synthetic Literature 10 | Mrozek-Wilczkiewicz et al., Bioorganic & Medicinal Chemistry, 18, 2664-26; 2010 |
| Synthetic Literature 11 | Fyfe et al., Angewandte Chemie, International Edition, 53(45), 12077-12080; 2014 |
| Synthetic Literature 12 | Wang et al., WO2015026792 |
| Synthetic Literature 13 | Deiters et al., WO2020123482 |

Available raw materials were obtained from the reagent suppliers listed in the texts. In addition, when the following reagent suppliers are mentioned in the tables, raw materials were purchased from the suppliers, and the concerned compounds were then synthesized using the purchased raw materials.

| | |
|---|---|
| Reagent Supplier 1 | Aurora Fine Chemicals |
| Reagent Supplier 2 | Asta Tech |
| Reagent Supplier 3 | Combi-Blocks |
| Reagent Supplier 4 | Tokyo Chemical Industry |
| Reagent Supplier 5 | BLD Pharmatech |
| Reagent Supplier 6 | Sigma-Aldrich |
| Reagent Supplier 7 | KANTO CHEMICAL |
| Reagent Supplier 8 | Santa Cruz Biotechnology |
| Reagent Supplier 9 | Enamine |
| Reagent Supplier 10 | FUJIFILM Wako Pure Chemical |
| Reagent Supplier 11 | Azepine |
| Reagent Supplier 12 | Milestone Pharma Tech |
| Reagent Supplier 13 | Apollo SCIENTIFIC |
| Reagent Supplier 14 | Chemieliva Pharmaceutical |
| Reagent Supplier 15 | Fluorochem |
| Reagent Supplier 16 | Matrix Scientific |
| Reagent Supplier 17 | Aurum Pharmatech |
| Reagent Supplier 18 | Oakwood Chemical |
| Reagent Supplier 19 | ChemBridge Corporation |
| Reagent Supplier 20 | PharmaBlock |

### Test Example 1

### Purification of Ras and Raf for Detection of Ras-Raf Binding Inhibitory Action

The in vitro Ras-Raf binding inhibitory activity of the compound of the present invention was evaluated by an ELISA method (Enzyme-Linked ImmunoSorbent Assay) described below.

Ras and Raf used upon the evaluation were purified and activated by the following method.

Using a pGEX6P-1 vector (GE Healthcare), human HRasG12V (full length, 1-189 amino acid residues) and human c-Raf-1 Ras binding domain (RBD) (50-131 amino acid residues) were each allowed to express as a fusion body with glutathione S-transferase (GST) in E. coli.

GST-HRasG12V-expressing cells were subjected to ultrasonication in a buffer {50 mM Tris-HCl pH 7.4, 150 mM NaCl, 5 mM MgCl₂, 1 mM ethylenediaminetetraacetic acid (EDTA), 1 mM dithiothreitol (DTT), 10% glycerol, and 1% Triton-X100}, and were then centrifuged at 100,000×g for 30 minutes, so as to collect the supernatant as a Ras protein fraction. HRasG12V in the supernatant was immobilized on a glutathione-agarose resin, and GST was then cleaved with PreScission Protease (GE Healthcare) for purification. The obtained HRasG12V was mixed with guanosine 5′-O-[gamma-thio]triphosphate trisodium salt (GTPTS) of 1000 times higher concentration at 30° C. for 1 hour, and thereafter, 20 mM MgCl₂ (final concentration) was added to stop the reaction and collect the GTPgS-loaded HRasG12V as a product.

On the other hand, GST-c-Raf-1 RBD-expressing cells were subjected to ultrasonication in the same buffer as described above, and were then centrifuged at 100,000×g for 30 minutes, so as to collected the supernatant as a GST-c-Raf-1 RBD protein fraction.

### Detection of Ras-Raf Binding Inhibitory Action

The in vitro Ras-Raf binding inhibitory activity of the compound of the present invention was evaluated by an ELISA method described below.

GST-c-Raf-1 RBD diluted with a Ras-Raf binding buffer (50 mM Tris-HCl pH7.4, 150 mM NaCl, 5 mM MgCl₂, 1 mM EDTA, and 1% Triton-X100) was added to each well of a glutathione-coated 96-well plate (Thermo Fisher Scientific.), and was incubated at 30° C. for 1 hour, to immobilize Raf on the well. The unbound free Raf was eliminated by washing the well with the Ras-Raf binding buffer three times. Subsequently, GTPTS-bound HRasG12V diluted with the Ras-Raf binding buffer and each compound solution (final DMSO concentration: 10%) were applied to each well, and the resulting mixture was then incubated at 30° C. for 1 hour, so that Ras and Raf were allowed to bind to each other. Thereafter, the plate was washed with the Ras-Raf binding buffer twice, and was then subjected to a blocking treatment at room temperature for 20 minutes, using TBS-Tween {10 mM Tris-HCl pH 7.4, 150 mM NaCl, and 0.05% (w/v) Tween-20}-5% (w/v) bovine serum albumin (BSA). After that, an anti-HRas antibody (C-20, Santa Cruz) or an anti-HRas antibody (259, Santa Cruz), which had been 1000 times diluted with TBS-Tween-5% BSA, was added to the reaction mixture, and incubated at room temperature for 1 hours. After the treatment with the primary antibody, the plate was washed with TBS-Tween-5% BSA three times, and a horseradish peroxidase-labeled secondary antibody against rabbit immunoglobulin G (GE Healthcare) of 1000 times diluted with TBS-Tween-5% BSA, was added to the plate, followed by incubation at room temperature for 1 hour. After the treatment with the secondary antibody, the plate was washed with TBS-Tween-5% BSA three times, and a substrate solution (TMB) was then added to the plate, followed by incubation at room temperature for 15 minutes for coloration. Finally, 2 M H₂SO₄ was added to stop color development (coloration reaction kit: Nacalai Tesque). The absorbance at 450 nm (OD₄₅₀) was measured, so that the color intensity was quantified. Inhibition by the test compound was determined according to the following equation:

$$Inhibition ( \% ) = ( {OD}_{450} \cdot control - {OD}_{450} \cdot compound ) / ( {OD}_{450} \cdot control - {OD}_{450} \cdot blank ) * 100.$$

The inhibitory activity of the test compound at each compound concentration was calculated according to the above calculational equation, and the concentration exhibiting inhibition of 50% of the maximum inhibition was calculated. The results are shown in the following table.

IC₅₀ (*) in which the anti-HRas antibody (259, Santa Cruz) was used

TABLE 4

| | ELISA IC50 (μM): 1st Ab_C-20; | |
|---|---|---|
| Example | 1st Ab_259 (*) | |
| 1 | 6.9 | |
| 2 | 7.9 | (*) |
| 13 | 2.5 | |
| 14 | 5.7 | |
| 15 | 7.3 | |
| 16 | 7.6 | |
| 17 | 9.6 | |
| 19 | 5.8 | |
| 20 | 10 | |
| 21 | 7.2 | |
| 22 | 13 | |
| 24 | 2.1 | |
| 25 | 5.9 | |
| 27 | 2.7 | |
| 28 | 0.88 | |
| 29 | 13 | |
| 31 | 13 | |
| 32 | 14 | |
| 33 | 3.1 | |
| 34 | 2.8 | |
| 35 | 2.6 | |
| 38 | 3.7 | |
| 39 | 8.3 | (*) |
| 40 | 6.2 | (*) |
| 41 | 4.7 | (*) |
| 42 | 2.8 | (*) |
| 43 | 7.1 | (*) |
| 44 | 0.96 | (*) |
| 45 | 1.2 | |
| 46 | 0.76 | |
| 47 | 3.9 | |
| 48 | 3.5 | |
| 49 | 13 | |
| 50 | 12 | |
| 51 | 2.1 | |
| 52 | 7.5 | |
| 53 | 0.88 | (*) |
| 54 | 3.4 | |
| 55 | 6.4 | |
| 56 | 0.57 | (*) |
| 58 | 0.7 | |
| 59 | 4 | |
| 60 | 6 | |
| 61 | 2.7 | |
| 62 | 5.6 | |
| 63 | 2 | |
| 64 | 1.1 | |
| 65 | 2.4 | |
| 66 | 2.3 | |
| 67 | 5.6 | |
| 68 | 2.3 | |
| 70 | 14 | |
| 71 | 2.4 | |
| 72 | 3.6 | (*) |
| 73 | 1.5 | (*) |
| 74 | 0.71 | (*) |
| 75 | 2.1 | (*) |
| 76 | 1.6 | (*) |
| 77 | 1.1 | (*) |
| 78 | 0.93 | (*) |
| 79 | 1.1 | (*) |
| 80 | 0.90 | (*) |
| 81 | 1.1 | (*) |
| 82 | 0.61 | (*) |
| 83 | 0.84 | (*) |
| 84 | 0.58 | (*) |
| 85 | 0.58 | (*) |
| 86 | 1.8 | |
| 87 | 2.1 | |
| 88 | 0.98 | |
| 89 | 0.5 | |
| 90 | 1.4 | |
| 91 | 0.87 | |
| 92 | 2.8 | |
| 94 | 0.66 | |
| 95 | 0.44 | |
| 96 | 0.35 | |
| 97 | 0.51 | |
| 98 | 0.68 | (*) |
| 99 | 0.78 | (*) |
| 100 | 0.77 | (*) |
| 101 | 0.78 | (*) |
| 102 | 4.1 | |
| 103 | 10 | |
| 104 | 9 | |
| 105 | 20 | |
| 106 | 4.1 | (*) |
| 107 | 1.7 | |
| 108 | 2.6 | (*) |
| 109 | 0.46 | (*) |
| 110 | 0.42 | (*) |
| 111 | 1.0 | (*) |
| 112 | 0.55 | (*) |
| 113 | 0.68 | (*) |
| 114 | 29 | |
| 115 | 11 | |
| 116 | 8.4 | |
| 117 | 1.1 | |
| 118 | 0.73 | |
| 119 | 2.9 | (*) |
| 120 | 2.2 | (*) |
| 123 | 8.5 | |
| 125 | 2.8 | |
| 126 | 1.4 | |
| 127 | 2.1 | |
| 128 | 0.49 | |
| 129 | 1.5 | (*) |
| 130 | 0.80 | (*) |
| 131 | 1.5 | |
| 132 | 1.2 | (*) |
| 133 | 1.8 | (*) |
| 134 | 1.1 | (*) |
| 135 | 0.79 | (*) |
| 136 | 1.7 | (*) |
| 137 | 1.3 | (*) |
| 138 | 0.88 | (*) |
| 139 | 6.0 | (*) |

| | |
|---|---|
| 140 | 1.3 |
| 141 | 1.2 |
| 142 | 0.8 |
| 143 | 0.6 |
| 144 | 0.8 |
| 145 | 0.6 |
| 146 | 0.5 |
| 147 | 0.5 |
| 148 | 0.3 |
| 149 | 0.3 |
| 152 | 0.9 |
| 153 | 1.1 |
| 155 | 0.7 |
| 156 | 0.9 |
| 157 | 0.6 |
| 158 | 0.8 |
| 159 | 0.3 |
| 162 | 2.4 |
| 163 | 0.7 |
| 164 | 1.2 |
| 167 | 0.8 |
| 168 | 1.3 |
| 169 | 0.9 |
| 170 | 0.5 |
| 171 | 0.4 |
| 173 | 0.4 |
| 174 | 0.5 |
| 175 | 0.5 |
| 176 | 1 |
| 177 | 0.8 |
| 178 | 0.8 |
| 179 | 1 |
| 180 | 1.5 |
| 181 | 2.4 |
| 182 | 0.9 |
| 183 | 1.4 |
| 184 | 0.9 |
| 185 | 1.6 |
| 186 | 1.9 |
| 187 | 0.5 |
| 188 | 0.9 |
| 189 | 1.5 |
| 190 | 0.9 |
| 191 | 1.1 |
| 192 | 0.8 |
| 193 | 0.7 |
| 194 | 0.9 |
| 195 | 0.9 |
| 196 | 0.8 |
| 199 | 2.1 |
| 200 | 1.0 |
| 201 | 0.8 |
| 203 | 0.6 |
| 204 | 1.8 |
| 205 | 0.7 |
| 206 | 2.0 |
| 207 | 0.7 |
| 208 | 1.3 |
| 209 | 0.4 |
| 210 | 0.6 |
| 211 | 0.6 |
| 212 | 0.8 |
| 213 | 0.9 |
| 214 | 28.9 |

The results of inhibition (%) obtained by the test compounds of 1 μM, 10 μM (*) or 100 μM (**) are shown in the following table.

TABLE 5

| | ELISA % inhibition at | |
|---|---|---|
| Example | 1 μM:10 μM (*):100 μM (**) | |
| 3 | 57 | (**) |
| 4 | 80 | (*) |
| 5 | 71 | (**) |
| 6 | 77 | (**) |
| 7 | 83 | (**) |
| 8 | 66 | (**) |
| 9 | 53 | (*) |
| 10 | 37 | (**) |
| 11 | 54 | (*) |
| 12 | 49 | (**) |
| 18 | 71 | (**) |
| 23 | 60 | (**) |
| 30 | 63 | (**) |
| 36 | 33 | (**) |
| 57 | 79 | (**) |
| 93 | 44 | (**) |
| 122 | 34 | (**) |
| 124 | 61 | (**) |
| 140 | 45.9 | |
| 141 | 46.2 | |
| 142 | 52.7 | |
| 143 | 44.3 | |
| 144 | 52.3 | |
| 145 | 60.4 | |
| 146 | 57.4 | |
| 147 | 66.1 | |
| 148 | 72.3 | |
| 149 | 76.6 | |
| 152 | 53.5 | |
| 153 | 63.1 | |
| 155 | 59.3 | |
| 156 | 52.0 | |
| 157 | 61.4 | |
| 158 | 59.2 | |
| 159 | 72.7 | |
| 162 | 47.2 | |
| 163 | 63.8 | |
| 164 | 48.7 | |
| 167 | 62.2 | |
| 168 | 48.4 | |
| 169 | 52.4 | |
| 170 | 66.8 | |
| 171 | 72.0 | |
| 173 | 73.5 | |
| 174 | 70.1 | |
| 175 | 67.2 | |
| 176 | 51.1 | |
| 177 | 58.0 | |
| 178 | 61.4 | |
| 179 | 50.0 | |
| 180 | 43.7 | |
| 184 | 53.3 | |
| 185 | 42.8 | |
| 186 | 39.3 | |
| 187 | 66.7 | |
| 188 | 53.7 | |
| 189 | 44.0 | |
| 190 | 55.1 | |
| 191 | 49.2 | |
| 192 | 57.0 | |
| 193 | 60.4 | |
| 194 | 55.4 | |
| 195 | 55.7 | |
| 196 | 58.0 | |
| 197 | 35.3 | (*) |

| | |
|---|---|
| 199 | 37.5 |
| 200 | 53.2 |
| 201 | 59.5 |
| 203 | 64.2 |
| 204 | 39.8 |
| 205 | 66.3 |
| 206 | 35.6 |
| 207 | 63.6 |
| 208 | 46.7 |
| 209 | 71.2 |
| 210 | 66.9 |
| 211 | 65.3 |
| 212 | 59.9 |
| 213 | 54.3 |

### Test Example 2

### Detection of Cell Growth Inhibitory Action

The cell growth inhibitory activity of the compound of the present invention on human culture cells having active-type mutation in Ras was evaluated by the below-mentioned method using suspension cancer cells {acute lymphoblastic leukemia cells CCRF-CEM (K-RasG12D), promyelocytic leukemia cells HL60 (N-RasQ61L), acute lymphoblastic leukemia cells MOLT4 (N-RasG12C), and small cell lung cancer cells SHP77 (K-RasG12V)} and adherent cancer cells {large bowel cancer cells SW480 (K-RasG12V) and large bowel cancer cell SW620 (K-RasG12V)}.

(Case of suspension cells) The cells suspended in a medium containing 0.5% (v/v) fetal bovine serum (FBS) and each compound solution (final DMSO concentration: 1%) were seeded on a 96-well plate (2 to 4×10⁴ cells/well), and the resulting mixture was then cultured at 37° C. in the presence of 5% CO₂ for 72 hours.

(Case of adherent cells) The cells suspended in a medium containing 10% FBS (1 to 2×10⁴ cells/well) were seeded on a 96-well plate, and were then cultured at 37° C. in the presence of 500 CO₂ overnight. Thereafter, the medium was exchanged with a medium containing 0.500 FBS, to which each compound solution (final DMSO concentration: 10%) had been added, and the cells were then cultured at 37° C. in the presence of 50% CO₂ for 72 hours.

Thereafter, the number of viable cells was measured using Cell Counting Reagent (Nacalai Tesque) in accordance with the manufacturer's instructions. Inhibition by the test compound was determined according to the following equation:

$$Inhibition ( \% ) = ( {OD}_{450} \cdot control - {OD}_{450} \cdot compound ) / ( {OD}_{450} \cdot control - {OD}_{450} \cdot blank ) * 100.$$

The results are shown in the following Table.

TABLE 6

| | CCRF-CEM (KRasG12D) % inhibition at | |
|---|---|---|
| Example | 1 μM, 3.3 μM(*), 10 μM (**), 3.2 μM (***) | |
| 1 | 51 | |
| 2 | 95 | (*) |
| 4 | 76 | |
| 9 | 99 | |
| 11 | 55 | |
| 13 | 33 | |
| 14 | 43 | |
| 15 | 99 | |
| 16 | 56 | |
| 17 | 94 | |
| 19 | 83 | |
| 21 | 97 | |
| 22 | 99 | (**) |
| 24 | 88 | |
| 27 | 80 | |
| 28 | 38 | |
| 33 | 94 | |
| 34 | 97 | |
| 35 | 40 | |
| 37 | 86 | (**) |
| 38 | 84 | (**) |
| 39 | 84 | (**) |
| 47 | 74 | |
| 48 | 69 | |
| 51 | 93 | (**) |
| 52 | 99 | |
| 54 | 59 | |
| 55 | 78 | (**) |
| 58 | 45 | |
| 59 | 99 | (**) |
| 60 | 99 | (**) |
| 61 | 35 | |
| 62 | 37 | |
| 63 | 83 | |
| 64 | 96 | |
| 65 | 54 | |
| 66 | 96 | |
| 67 | 44 | |
| 68 | 80 | (*) |
| 71 | 82 | (**) |
| 102 | 30 | (**) |
| 104 | 70 | |
| 107 | 81 | (**) |
| 114 | 99 | |
| 115 | 98 | (**) |
| 116 | 98 | (**) |
| 123 | 47 | |
| 125 | 39 | |
| 126 | 77 | |
| 127 | 98 | (**) |
| 130 | 73 | |
| 131 | 77 | |
| 132 | 38 | |
| 133 | 80 | (**) |
| 149 | 95.0 | (***) |
| 181 | 98.2 | (***) |
| 182 | 97.9 | (***) |
| 183 | 95.2 | (***) |
| 190 | 95.3 | (***) |
| 198 | 68.7 | (***) |
| 199 | 45.0 | (***) |
| 200 | 96.8 | (***) |
| 201 | 96.8 | (***) |
| 206 | 97.3 | (***) |
| 207 | 97.8 | (***) |
| 208 | 97 | (***) |
| 209 | 97.8 | (***) |
| 210 | 97.5 | (***) |
| 211 | 97.4 | (***) |
| 213 | 89.3 | (***) |
| 214 | 96.0 | (***) |

TABLE 7

| | HL-60 (NRasQ61L) % inhibition at | |
|---|---|---|
| Example | 1 μM, 1.1 μM (*), 3.3 μM (**), 10 μM (***), 3.2 μM (****) | |
| 1 | 95 | (***) |
| 2 | 99 | (***) |
| 21 | 43 | |
| 28 | 97 | (***) |
| 38 | 31 | |
| 39 | 79 | (***) |
| 44 | 95 | (***) |
| 45 | 98 | (***) |
| 46 | 31 | (*) |
| 47 | 89 | |
| 48 | 100 | (***) |
| 53 | 100 | (***) |
| 54 | 97 | (***) |
| 55 | 96 | (***) |
| 56 | 96 | (***) |
| 58 | 99 | (***) |
| 62 | 97 | (***) |
| 64 | 87 | |
| 68 | 37 | |
| 70 | 33 | |
| 71 | 41 | |
| 74 | 99 | (***) |
| 75 | 95 | (***) |
| 77 | 94 | (***) |
| 78 | 92 | (***) |
| 80 | 38 | |
| 82 | 96 | (***) |
| 83 | 68 | |
| 84 | 96 | |
| 85 | 48 | |
| 86 | 63 | (**) |
| 87 | 94 | (***) |
| 88 | 89 | (**) |
| 89 | 95 | (*) |
| 91 | 97 | (**) |
| 92 | 87 | (**) |
| 94 | 96 | (**) |
| 95 | 88 | (*) |
| 96 | 93 | (*) |
| 97 | 91 | (*) |
| 98 | 100 | (***) |
| 99 | 84 | (***) |
| 100 | 87 | (***) |
| 101 | 87 | (***) |
| 107 | 82 | (***) |
| 109 | 76 | |
| 110 | 41 | |
| 112 | 39 | |
| 113 | 54 | (***) |
| 117 | 94 | (**) |
| 118 | 98 | (**) |
| 128 | 96 | (**) |
| 130 | 44 | |
| 131 | 85 | |
| 132 | 42 | |
| 133 | 99 | (***) |
| 135 | 98 | (***) |
| 138 | 65 | (**) |
| 140 | 95.3 | (****) |
| 141 | 94.6 | (****) |
| 143 | 97.2 | (****) |
| 144 | 95.9 | (****) |
| 145 | 99.7 | (****) |
| 146 | 99.3 | (****) |
| 147 | 59.1 | (****) |
| 149 | 96.9 | (****) |
| 181 | 96.7 | (****) |
| 182 | 97.5 | (****) |
| 183 | 79.5 | (****) |
| 190 | 96.4 | (****) |
| 198 | 75.1 | (****) |
| 199 | 66.2 | (****) |
| 200 | 96.8 | (****) |
| 201 | 97.3 | (****) |
| 206 | 97.6 | (****) |
| 207 | 93.1 | (****) |
| 208 | 95.1 | (****) |
| 209 | 96.7 | (****) |
| 210 | 97.3 | (****) |
| 211 | 97.2 | (****) |
| 213 | 96.5 | (****) |
| 214 | 36.7 | (****) |

TABLE 8

| | MOLT-4 (NRasG12C) % inhibition at | |
|---|---|---|
| Example | 1 μM, 3.3 μM (*), 10 μM (**), 3.2 μM (***) | |
| 1 | 88 | (*) |
| 2 | 99 | (**) |
| 4 | 100 | (**) |
| 9 | 51 | |
| 11 | 100 | (**) |
| 13 | 100 | (**) |
| 14 | 100 | (**) |
| 15 | 98 | |
| 16 | 100 | (**) |
| 17 | 32 | |
| 19 | 100 | (**) |
| 21 | 100 | (*) |
| 22 | 100 | (**) |
| 24 | 54 | |
| 25 | 100 | (**) |
| 27 | 100 | (**) |
| 51 | 40 | (**) |
| 52 | 96 | |
| 54 | 100 | (**) |
| 104 | 100 | (**) |
| 123 | 100 | (**) |
| 125 | 100 | (**) |
| 144 | 93.8 | (***) |
| 149 | 95.5 | (***) |
| 181 | 96.3 | (***) |
| 182 | 96.9 | (***) |
| 183 | 90.8 | (***) |
| 190 | 96.6 | (***) |
| 200 | 96.2 | (***) |
| 201 | 95.4 | (***) |
| 206 | 96 | (***) |
| 207 | 96.1 | (***) |
| 208 | 96.2 | (***) |
| 209 | 96.7 | (***) |
| 210 | 96.2 | (***) |
| 211 | 96.5 | (***) |
| 213 | 96.4 | (***) |

TABLE 9

| | SHP77 (KRasG12V) % inhibition at |
|---|---|
| Example | 1 μM, 3.3 μM (*), 3.2 μM (**) |
| 1 | 73 |
| 47 | 61 |
| 64 | 95 |
| 68 | 67 (*) |
| 131 | 73 |
| 149 | 55.2 (**) |
| 152 | 81.1 (**) |
| 153 | 65.7 (**) |
| 156 | 35.1 (**) |
| 157 | 49.2 (**) |
| 158 | 72.4 (**) |
| 159 | 52.6 (**) |
| 162 | 60.5 (**) |
| 163 | 72.3 (**) |
| 164 | 43.5 (**) |
| 167 | 65.8 (**) |
| 168 | 53.1 (**) |
| 169 | 51.5 (**) |
| 172 | 99.7 (**) |
| 178 | 76.9 (**) |
| 180 | 69.2 (**) |
| 187 | 66.8 |
| 188 | 76.6 (***) |
| 190 | 92.2 (**) |
| 192 | 58.3 (**) |
| 193 | 98.1 (**) |
| 194 | 58.3 (**) |
| 195 | 96.4 (**) |
| 196 | 99.5 (**) |
| 197 | 65.7 (**) |
| 202 | 63.6 (**) |
| 203 | 77.8 (**) |
| 205 | 50.7 (***) |
| 213 | 95.5 (**) |

TABLE 10

| Example | SW480 (KRasG12V) % inhibition at 10 μM |
|---|---|
| 1 | 50 |
| 47 | 51 |
| 64 | 55 |
| 68 | 52 |
| 131 | 56 |

TABLE 11

| | SW620 (KRasG12V) % inhibition at |
|---|---|
| Example | 1 μM, 3.3 μM (*), 10 μM (**), 3.2 μM (***) |
| 1 | 53 |
| 45 | 66 |
| 46 | 84 |
| 75 | 79 |
| 77 | 68 |
| 80 | 36 |
| 82 | 52 |
| 83 | 71 |
| 84 | 90 |
| 85 | 74 |
| 86 | 43 |
| 87 | 32 |
| 88 | 46 |
| 89 | 71 |
| 91 | 70 |
| 92 | 40 |
| 94 | 82 |
| 95 | 70 |
| 96 | 86 |
| 97 | 71 |
| 113 | 69 (*) |
| 117 | 66 |
| 118 | 53 |
| 128 | 70 |
| 140 | 93.9 (***) |
| 141 | 91.3 (***) |
| 142 | 34.4 (***) |
| 143 | 79.3 (***) |
| 144 | 97.9 (***) |
| 145 | 91.8 (***) |
| 146 | 92.8 (***) |
| 147 | 92.2 (***) |
| 148 | 98.9 (***) |
| 149 | 99.2 (***) |
| 152 | 97.2 (***) |
| 153 | 96.1 (***) |
| 156 | 99.0 (***) |
| 157 | 97.5 (***) |
| 158 | 58.5 (***) |
| 159 | 96.6 (***) |
| 163 | 34.7 (***) |
| 167 | 64.4 (***) |
| 169 | 35.1 (***) |
| 170 | 41.6 (***) |
| 171 | 79.5 (***) |
| 172 | 97.0 (***) |
| 173 | 67.0 (***) |
| 177 | 97.1 (***) |
| 178 | 53.6 (***) |
| 181 | 74.3 (***) |
| 182 | 96.9 (***) |
| 183 | 34.8 (***) |
| 184 | 45.4 (***) |
| 187 | 39.3 |
| 188 | 79.3 (***) |
| 190 | 99.9 (***) |
| 192 | 45.7 (***) |
| 193 | 96.1 (***) |
| 195 | 95.4 (***) |
| 196 | 94.3 (***) |
| 197 | 35.5 (***) |
| 198 | 30.4 (***) |
| 199 | 51.2 (***) |
| 200 | 91.7 (***) |
| 201 | 95.1 (***) |
| 202 | 96.6 (***) |
| 203 | 68.2 (***) |
| 205 | 37.0 (***) |
| 206 | 98.1 (***) |
| 207 | 70.3 (***) |
| 208 | 81.1 (***) |
| 209 | 94.4 (***) |
| 210 | 78.0 (***) |
| 211 | 90.9 (***) |
| 213 | 98.5 (***) |
| 214 | 92.2 (***) |

### Test Example 3

### Detection of Cell Growth Inhibitory Action Against Drug-Resistant Malignant Melanoma

The human malignant melanoma cell lines A375 and HT 144, each having BRafV600E mutation, were cultured in a medium containing 10% FBS at 37° C. in the presence of 5% CO₂. At the time point in which the cells became confluent, 1 μM Vemurafenib, a BRaf inhibitor, was added to the medium, and the cells were then cultured in the presence of Vemurafenib for 1 month or more (during which the medium was exchanged every 1 week), so as to acquire Vemurafenib-resistant malignant melanoma cells A375R and HT 144R. The resulting resistant cells were suspended in a medium containing 10% FBS, and then seeded on a 96-well plate to an amount of 1 to 2×10⁴ cells/well. The cells were cultured at 37° C. in the presence of 5% CO₂ overnight. Thereafter, the medium was exchanged with a medium containing 0.5% FBS, to which each compound solution (final DMSO concentration: 1%) and 1 μM Vemurafenib had been added, and the cells were then cultured at 37° C. in the presence of 5% CO₂ for 72 hours.

Thereafter, the number of viable cells was measured using Cell Counting Reagent (Nacalai Tesque) in accordance with the manufacturer's instructions. Inhibition by the test compound was determined according to the following equation:

$$Inhibition ( \% ) = ( {OD}_{450} \cdot control - {OD}_{450} \cdot compound ) / ( {OD}_{450} \cdot control - {OD}_{450} \cdot blank ) * 100.$$

Hereafter, Inhibition (%) by 3.3 μM compound is shown.

TABLE 12

| | A375R (BRafV600E) | HT144R (BRafV600E) |
|---|---|---|
| | % inhibition at 3.3 μM | % inhibition at 3.3 μM |
| Example | (in the presence of 1 μM PLX) | (in the presence of 1 μM PLX) |
| 1 | 28 | 66 |
| 47 | 90 | 92 |
| 131 | 32 | 26 |

### Test Example 4

### Detection of Ras-Raf Signaling Inhibitory Action at Culture Cell Level

The Ras-Raf signaling inhibitory activity of the compound of the present invention at a culture cell level was evaluated by the following method.

The HL60 cells described in Test Example 2 suspended in a medium containing 0.5% (v/v) FBS with each compound solution (final DMSO concentration: 1%), were seeded on a 12-well plate (2 to 4×10⁵ cells/well), and the resulting mixture was then cultured at 37° C. in the presence of 5% CO₂ for 3 hours.

After completion of the cell culture, proteins were extracted from the cells, using RIPA Buffer (Nacalai Tesque) containing a mixture of protease inhibitors and phosphatase inhibitors. An equivalent amount of protein was separated by standard SDS-PAGE and was then transferred onto a PVDF membrane. In order to detect the activation (phosphorylation) of MEK and ERK, downstream molecules of the Ras-Raf signaling pathway, the membrane was probed with 1000-fold diluted primary antibodies {phosphorylated MEK (pMEK: #9121), phosphorylated ERK (pERK: #9101), total MEK (tMEK: #9122), and total ERK (tERK: 9102); all from Cell signaling}, and then with a 1000-fold diluted horseradish peroxidase-labeled secondary antibody against rabbit immunoglobulin G. The immunoreactive signals were colored with EzWestLumi One (ATTO), and were then detected by Fusion FX (Vilber). Signal intensity was quantified by pixel counting. Inhibition by the test compound was determined according to the following equation:

$$Inhibition ( \% ) = ( signal intensity of p / t MEK or ERK of control -$$

$$signal intensity of p / t MEK or ERK of compound ) /$$

$$( signal intensity of p / t MEK or ERK of control -$$

$$signal itensity of p / t MEK or ERK of blank ) * 100.$$

The results of inhibition (%) by 1 μM test compound are shown in the following table.

TABLE 13
HL60 (N-RasQ61L)

| Example | pMEK % inhibition | pERK % inhibition |
|---|---|---|
| 1 | 30 | 61 |
| 47 | 88 | 94 |
| 48 | 81 | 96 |
| 58 | 72 | 100 |
| 68 | 68 | 93 |
| 84 | 58 | 63 |
| 109 | 15 | 71 |
| 131 | 76 | 99 |

### Test Example 5

### Detection of Ras/Raf Signaling Inhibition in Animal Models

Detection of Ras/Raf signaling inhibition in animal models was evaluated by the following method.

Human culture cells (5×10⁶ cells) were subcutaneously injected into the right flank of female thymus-free nude mice (6- to 8-week-old; CLEA Japan, Inc.). After the tumor size had reached approximately 50 mm³ on average, 30 to 160 mg/kg compound suspended in a diluted compound solution {HCO—40 (8.75%), Cremophor EL (17.5%), EtOH (8.75%), DMSO (15%), and a phosphate buffered saline (50%)} was intraperitoneally administered to the mice for 21 days on 5 consecutive days per week. Twenty-four hours after the final administration of the compound, the tumors were dissected and weighed. Inhibition by the tested compound was determined by the following equation:

$$Inhibition ( \% ) = ( 1 - tumor weight with compound administration /$$

$$tumor weight with vehicle administration ) * 100.$$

The results are shown in the following table.

TABLE 14

| | | | | | A375R |
|---|---|---|---|---|---|
| | | SHP77 | SW480 | HL60 | (BRafV600E- |
| Example | mg/kg | (KRasG12V) | (KRasG12V) | (NRasQ61L) | resistance) |
| 1 | 30 | 20.5 | | 16.5 | *24.4 |
| | 80 | | 29.8 | | |
| | 90 | 33.4 | | 57.4 | *31.4 |
| | 160 | | 55.0 | | |
| 64 | 30 | 31.6 | | 17.8 | |
| | 90 | 65.2 | | 62.1 | |
| 131 | 30 | 32.7 | | 34.2 | |
| | 60 | 54.5 | | 67.3 | |
| 68 | 30 | | | 17.6 | |
| | 90 | | | 14.2 | |
| 47 | 30 | 21.5 | | 56.6 | *37.2 |
| | 75 | 37.5 | | 49 | *60.5 |

*in the presence of 30 mg/kg PLX4072

1. A compound represented by the following formula (I) or a pharmaceutically acceptable salt thereof, or an isomer thereof: 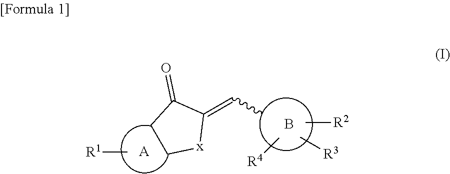
wherein
A represents a benzene ring or a pyridine ring,
B represents a C₆₋₁₀ aryl group, or a heteroaryl group containing 1 to 4 atoms selected from N, S and O,
X represents —NR⁵—,
R¹ represents H, a C₁₋₆ alkyl group optionally substituted with a NHCOCH₃ group, or halogen,
R², R³ and R⁴, which are the same or different, each represent (provided that at least any one of them is not H)
H;
C₁₋₆ alkyl-SO₂—;
cyano;
halogen;
nitro;
azide;
a —CO—R¹¹ group (wherein the R¹ represents
OH,
an R¹² group (wherein the R¹² group represents a heterocyclyl group containing 1 or 2 atoms (groups) selected from N, S, SO, SO₂ and O, and optionally having a substituent),
NH₂, NHR¹², or N(R¹²)R¹², or
C₆₋₁₀ arylamino);
an —O—CH₂—CO—R¹¹ group;
a C₁₋₆ alkyl group optionally having a substituent
(wherein the substituent represents
—CONH₂,
an R¹² group,
OH,
—NR¹³R¹⁴ (wherein the R¹³ represents C₁₋₆ alkyl-SO₂—, C₁₋₆ alkyl-CO—, or an R¹² group, and the R¹⁴ represents H, a C₁₋₆ alkyl group, or an R¹² group),
R¹²—CO—, or
R¹²—C₁₋₆ alkyl-CONH—);
an —OR¹⁵ group
(wherein the R¹⁵ represents
H,
a C₁₋₆ alkyl group optionally having a substituent
(wherein the substituent represents OH, C₁₋₆ alkoxy, C₆₋₁₀ aryl, C₆₋₁₀ aryloxy, R¹², cyano, C₁₋₆ alkyl-SO₂—, or methylsulfinyl),
C₁₋₆ alkyl-SO₂—, or
a heteroaryl group containing 1 to 4 atoms selected from N, S and O);
an —NR¹⁶R¹⁷ group
(wherein the R¹⁶ and R¹⁷, which are the same or different, each represent
H,
C₁₋₆ alkyl-CO—,
an R¹² group, or
R¹²—C₁₋₆ alkyl-CO—, or R¹⁶ and R¹⁷ may form an R¹² group, together with N);
a C₆₋₁₀ aryl group optionally having a substituent
(wherein the substituent represents C₆₋₁₀ aryloxy, a heteroaryl group containing 1 to 4 atoms selected from N, S and O, and optionally having a substituent, or phenyl optionally annelated with R¹²);
a heteroaryl group containing 1 to 4 atoms selected from N, S and O, and optionally having a substituent; or
—(CH═CH)—R¹⁸ (wherein the R¹⁸ represents —CO—R¹⁹ or —SO₂—R¹⁹, and the R¹⁹ represents NH₂, or an R¹² group); wherein
R⁵ represents C₁₋₆ alkyl, —COR⁶, —COOR⁶, —CONR⁶R⁷, —SOnR⁶, or SOnNR⁶R⁷, wherein n represents 0, 1 or 2, and
R⁶ and R⁷, which are the same or different, each represent H, a C₁₋₆ alkyl group, or a C₆₋₁₀ aryl group, and the wavy line represents a geometrical isomer.

2. The compound according to claim 1 or a pharmaceutically acceptable salt thereof, or an isomer thereof, wherein B represents a heteroaryl group containing 1 to 4 atoms selected from N, S and O.

3. The compound according to claim 1 or a pharmaceutically acceptable salt thereof, or an isomer thereof, wherein B represents pyridyl,
quinolyl,
indolyl,
thiazolyl,
pyrrolopyridinyl,
benzothiazolyl, or
furopyridinyl.

4. The compound according to claim 1 or a pharmaceutically acceptable salt thereof, or an isomer thereof, wherein B represents the following: 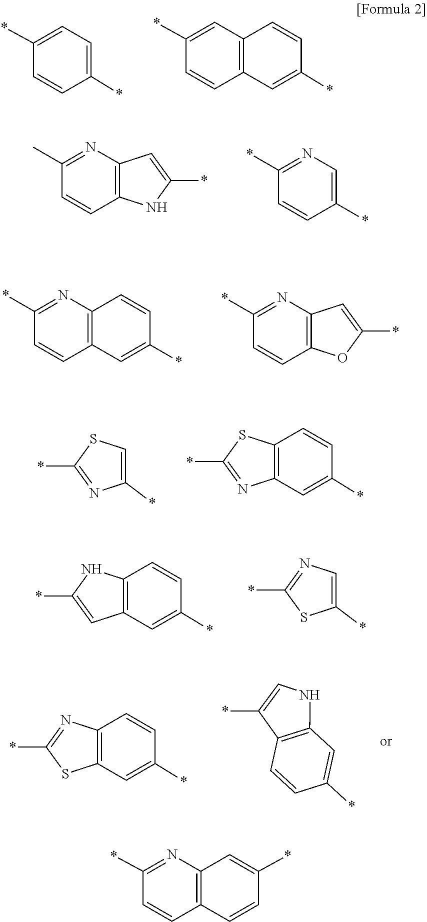
provided that one of the bonding hands corresponds to the bonding of the wavy line in the formula (I), and the other bonding hand represents a bonding hand with any one substituent other than the hydrogen in R², R³ and R⁴, and
in such a case, if there is another substituent, the substituent is substituted with the residual position described above.

5. The compound according to claim 1 or a pharmaceutically acceptable salt thereof, or an isomer thereof, wherein B represents
quinolyl,
thiazolyl, or
benzothiazolyl.

6. The compound according to claim 5 or a pharmaceutically acceptable salt thereof, or an isomer thereof, wherein the C₆₋₁₀ aryl group in the C₆₋₁₀ aryl group optionally having a substituent, as represented in R², R³ and R⁴, represents phenyl or naphthyl, and the substituent represents a heteroaryl group containing 1 to 4 atoms selected from N, S and O, and optionally having a substituent, or phenyl optionally annelated with an R¹² group.

7. The compound according to claim 5 or a pharmaceutically acceptable salt thereof, or an isomer thereof, wherein the heteroaryl group containing 1 to 4 atoms selected from N, S and O, and optionally having a substituent, as represented in R², R³ and R⁴, represents pyridyl, phenylpyridyl, quinolyl, indazolyl, pyrazolyl, or methylpyrazolyl.

8. The compound according to claim 7 or a pharmaceutically acceptable salt thereof, or an isomer thereof, wherein the heterocyclyl group containing 1 or 2 atoms (groups) selected from N, S, SO, SO₂ and O, in the heterocyclyl group containing 1 or 2 atoms (groups) selected from N, S, SO, SO₂ and O, and optionally having a substituent, as represented in R¹², represents
morpholino,
piperazinyl,
thiomorpholino,
dioxidothiomorpholino,
tetrahydropyranyl,
tetrahydrothiopyranyl,
pyrrolidinyl,
dioxidotetrahydrothiopyranyl, or
piperidinyl.

9. The compound according to claim 1 or a pharmaceutically acceptable salt thereof, or an isomer thereof, wherein the substituent in the heterocyclyl group containing 1 or 2 atoms (groups) selected from N, S, SO, SO₂ and O, and optionally having a substituent, as represented in R¹², represents
—CO—,
—COOH,
cyano,
1 or 2 C₁₋₆ alkyl groups,
C₁₋₆ alkyl-CO—,
C₁₋₆ alkyl-SO₂—,
a C₆₋₁₀ aryl group,
3-methoxy-2-hydroxypropyl,
an R¹² group,
R¹²—CH₂—,
R¹²—CH₂CO—,
R¹²—CH₂OCO—, or
methoxyethyl.

10. The compound according to claim 1 or a pharmaceutically acceptable salt thereof, or an isomer thereof, wherein the wavy line portion of the compound represented by the formula (I) represents a Z-form.

11. The compound according to claim 1 or a pharmaceutically acceptable salt thereof, or an isomer thereof, wherein the wavy line portion of the compound represented by the formula (I) represents an E-form.

12. The compound according to claim 1 or a pharmaceutically acceptable salt thereof, or an isomer thereof, wherein
the group represented by the following formula (II) in the formula (I): 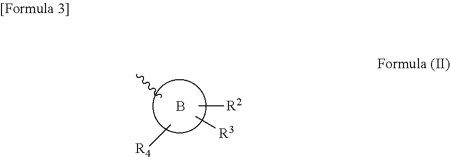
wherein the wavy line represents a bonding hand,
is a group selected from the following:

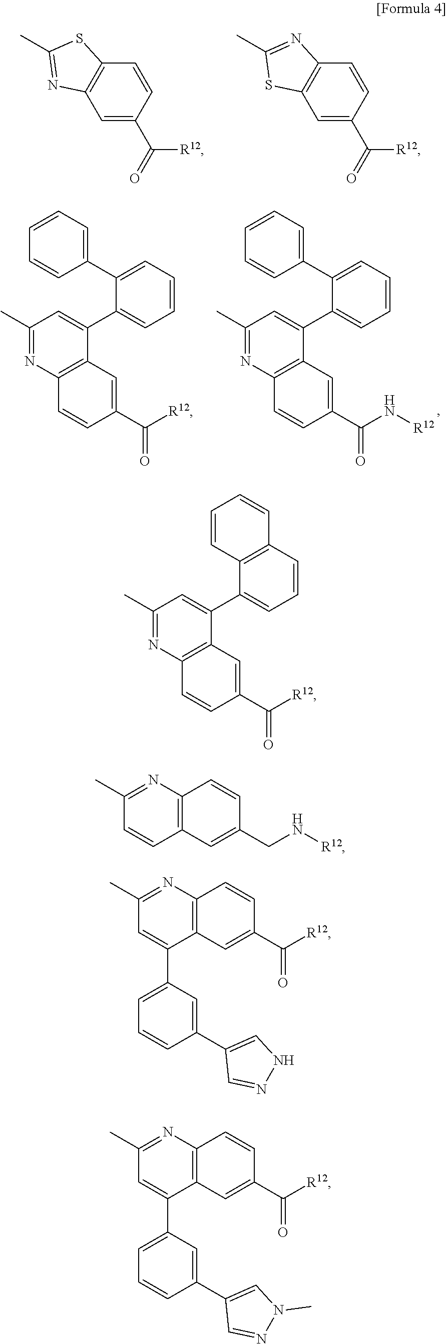

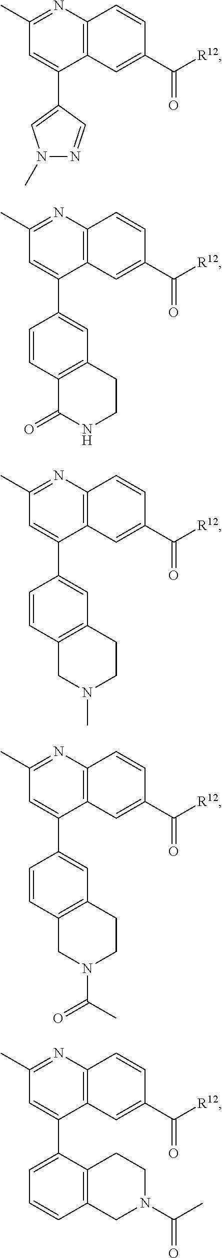

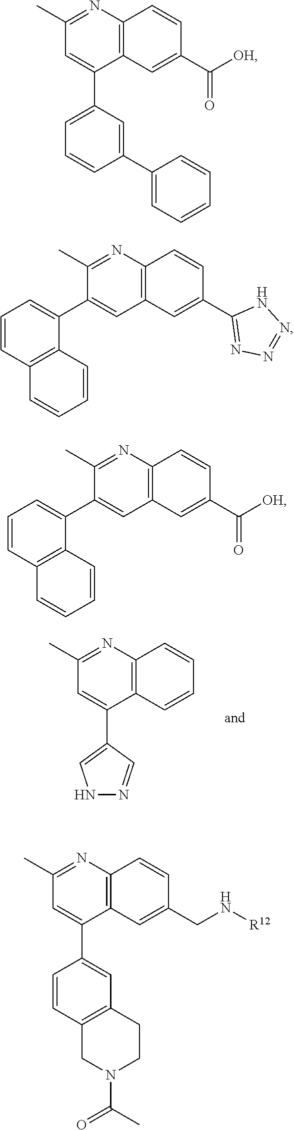

13. A Ras/Raf binding inhibitor, comprising the compound according to claim 1 or a pharmaceutically acceptable salt thereof, or an isomer thereof.

14. A medicament, comprising the compound according to claim 1 or a pharmaceutically acceptable salt thereof, or an isomer thereof.

15. An anticancer agent, comprising the compound according to claim 1 or a pharmaceutically acceptable salt thereof, or an isomer thereof.
